(19) United States
(12) Patent Application Publication
(10) Pub. No.: US 2024/0150339 A1
(43) Pub. Date: May 9, 2024

(54) Compounds and Their Use as PDE4 Activators

(71) Applicant: Mironid Limited, Glasgow (GB)

(72) Inventors: Julia Mary Adam, Glasgow (GB); David Roger Adams, Glasgow (GB)

(73) Assignee: Mironid Limited, Glasgow (GB)

(21) Appl. No.: 18/276,309

(22) PCT Filed: Feb. 15, 2022
(86) PCT No.: PCT/GB2022/050402

§ 371 Date: Aug. 8, 2023

(30) Foreign Application Priority Data
Feb. 15, 2021 (GB) 2102088.8

(51) Int. Cl. C07D 417/04; A61P 13/12; C07D 277/82; C07D 417/14; C07D 471/04; C07D 471/18; C07D 471/20; C07D 498/10

(52) U.S. Cl. CPC C07D 417/04; A61P 13/12; C07D 277/82; C07D 417/14; C07D 471/04; C07D 471/18; C07D 471/20; C07D 498/10

(57) ABSTRACT

The present invention relates to compounds of Formulas A-D, I-IV and Z, their use as activators of long form cyclic nucleotide phosphodiesterase-4 (PDE4) enzymes (isoforms) and to these compounds for use in a method for the treatment or prevention of disorders requiring a reduction of second messenger responses mediated by cyclic 3′,5′-adenosine monophosphate (cAMP).

### FIELD OF THE INVENTION

The present invention relates to compounds as defined herein, their use as activators of long form cyclic nucleotide phosphodiesterase-4 (PDE4) enzymes (isoforms) and to therapies using these compounds. In particular, the invention relates to these compounds for use in a method for the treatment or prevention of disorders requiring a reduction of second messenger responses mediated by cyclic 3′,5′-adenosine monophosphate (cAMP).

### BACKGROUND TO THE INVENTION

Cyclic 3′,5′-adenosine monophosphate—“cAMP”—is a critical intracellular biochemical messenger that is involved in the transduction of the cellular effects of a variety of hormones, neurotransmitters, and other extracellular biological factors in most animal and human cells.

The intracellular concentration of cAMP is controlled by the relative balance between its rate of production and degradation. cAMP is generated by biosynthetic enzymes of the adenylyl cyclase superfamily and degraded by members of the cyclic nucleotide phosphodiesterase (PDE) superfamily. Certain members of the PDE superfamily, such as PDE4, specifically degrade cAMP, while others either specifically degrade cyclic guanosine monophosphate (cGMP) or degrade both cAMP and cGMP. PDE4 enzymes inactivate cAMP, thereby terminating its signalling, by hydrolysing cAMP to 5′-AMP (Lugnier, C. Pharmacol Ther. 109: 366-398, 2006).

Four PDE4 genes (PDE4A, PDE4B, PDE4C and PDE4D) have been identified, each of which encodes a number of different enzyme isoforms through the use of alternative promoters and mRNA splicing. On the basis of their primary structures, the catalytically active PDE4 splice variants can be classified as “long”, “short” or “super-short” forms (Houslay, M. D. Prog Nucleic Acid Res Mo! Biol. 69: 249-315, 2001). A “dead short” form also exists, which is not catalytically active (Houslay, M. D., Baillie, G. S. and Maurice, D. H. Circ Res. 100: 950-66, 2007). PDE4 long forms have two regulatory regions, called upstream conserved regions 1 and 2 (UCR1 and UCR2), located between their isoform-specific N-terminal portion and the catalytic domain. The UCR1 domain is absent in short forms, whereas the super-short forms not only lack UCR1, but also have a truncated UCR2 domain (Houslay, M. D., Schafer, P. and Zhang, K. Drug Discovery Today 10: 1503-1519, 2005).

PDE4 long forms, but not short forms, associate into dimers within cells (Richter, W and Conti, M. J. Biol. Chem. 277: 40212-40221, 2002; Bolger, G. B. et al., Cell. Signal. 27: 756-769, 2015). A proposed negative allosteric modulation of PDE4 long forms by small molecules has been reported (Burgin A. B. et al., Nat. Biotechnol. 28: 63-70, 2010; Gurney M. E. et al., Handb. Exp. Pharmacol. 204: 167-192, 2011).

It is known in the art that PDE4 long forms may be activated by endogenous cellular mechanisms, such as phosphorylation (MacKenzie, S. J. et al., Br. J. Pharmacol. 136: 421-433, 2002) and phosphatidic acid (Grange et al., J. Biol. Chem. 275: 33379-33387, 2000). Activation of PDE4 long forms by ectopic expression of a 57 amino acid protein (called ‘UCR1C’) whose precise sequence reflects part of that of the upstream conserved region 1 of PDE4D (‘UCR1C’ sequence reflects that of amino acids 80-136 while UCR is amino acids 17-136: numbering based on the PDE4D3 long isoform) has been reported (Wang, L. et al., Cell. Signal. 27: 908-922, 2015: “UCR1C is a novel activator of phosphodiesterase 4 (PDE4) long isoforms and attenuates cardiomyocyte hypertrophy”). The authors hypothesised that PDE4 activation might be used as a potential therapeutic strategy for preventing cardiac hypertrophy.

The first small molecules that act as activators of PDE4 long forms were recently disclosed in WO2016151300, WO2018060704 and WO2019193342. A small molecule activator of PDE4 long forms was recently evaluated in cell-based models of Autosomal Dominant Polycystic Kidney Disease (ADPKD) (Omar et al., PNAS 116: 13320-13329, 2019). No small molecule activators of PDE4 long forms have yet been reported in clinical development. There remains a need for further, structurally distinct small molecule activators of PDE4 long forms for potential development as therapeutic agents.

It is amongst the objects of the present invention to provide new small molecule activators of at least one of the long forms of PDE4 for use in a method of therapy, as well as specific disease treatment or prevention.

### SUMMARY OF THE INVENTION

In a first aspect of the present invention, there is provided a compound of Formula A, for example a compound of Formula I:

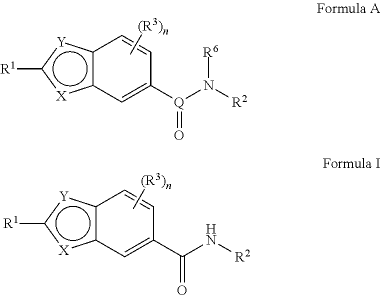

or a pharmaceutically acceptable salt or derivative thereof, wherein:
   one of X and Y is S and the other is N;
   Q is C or S(O);
   R¹ is a 4- to 10-membered monocyclic, bridged or bicyclic ring containing at least 1 ring N heteroatom and optionally a ring 0 heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring 0 heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof, wherein a straight chain portion of said (C3-8)alkyl group may be optionally interrupted by 1—O—;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
   R⁶ is H or (C1-6)alkyl; and
   n is 0, 1,2 or 3;
for use in the treatment or prevention of a disease or disorder that can be ameliorated by activation of long isoforms of PDE4.

In a second aspect of the present invention, there is provided a compound of Formula B, for example a compound of Formula II

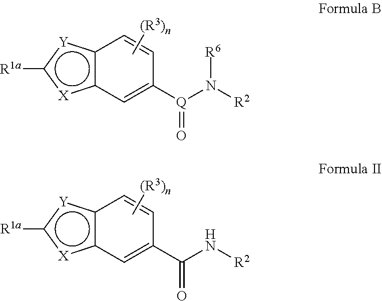

or a pharmaceutically acceptable salt or derivative thereof, wherein:
   one of X and Y is S and the other is N;
   Q is C or S(O);
   R^{1a} is a 4- to 10-membered non-aromatic, monocyclic, bridged or bicyclic ring containing at least 1 ring N heteroatom and optionally a ring 0 heteroatom, wherein at least 1 ring N heteroatom is not at the point of attachment of R^{1a}, and wherein R^{1a} is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring 0 heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof, wherein a straight chain portion of said (C3-8)alkyl group may be optionally interrupted by 1—O—;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH;
   R⁶ is H or (C1-6)alkyl; and
   n is 0, 1,2 or 3;
   wherein when R^{1a} is 4-cyclopentylpiperazin-1-yl, 4-cyclopropylpiperazin-1-yl or 4-isopropylpiperazin-1-yl, Q when present is C and n is 0, R² is not unsubstituted, uninterrupted, straight chain or branched (C3-6)alkyl or unsubstituted (C3-8)cycloalkyl; and
   wherein the compound is not 2-(1-piperazinyl)-N-propyl-6-benzothiazolecarboxamide, N-(1-methylethyl)-2-(1-piperazinyl)-6-benzothiazolecarboxamide or N-cyclopropyl-2-(1-piperazinyl)-6-benzothiazolecarboxamide. An “uninterrupted” (C3-6)alkyl is not interrupted by 1—O—.

In a third aspect of the present invention, there is provided a compound of Formula C, for example a compound of Formula III

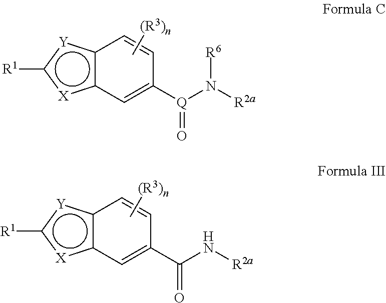

or a pharmaceutically acceptable salt or derivative thereof, wherein:
   one of X and Y is S and the other is N;
   Q is C or S(O);
   R¹ is a 4- to 10-membered monocyclic, bridged or bicyclic ring containing at least 1 ring N heteroatom and optionally a ring 0 heteroatom, and wherein R¹ is optionally substituted with 1 or more R⁴;
   R^{2a} is
   (i) (C5-7)cycloalkyl fused to a 6-membered aromatic or heteroaromatic ring that contains 0, 1 or 2 ring N atoms;
   (ii) a 5- to 7-membered non-aromatic heterocycle containing one ring 0 heteroatom, optionally fused to a 6-membered aromatic or heteroaromatic ring that contains 0, 1 or 2 ring N atoms;
   (iv) a (4-6)cycloalkyl group;
   and wherein R^{2a} is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
   R⁶ is H or (C1-6)alkyl; and
   n is 0, 1, 2 or 3; and
   wherein when R^{2a} is (iv) a (C4-6)cycloalkyl group it is substituted by at least 2 R⁵; and
   wherein the compound is not 2-(4-morpholinyl)-N-(1,2,3,4-tetrahydronaphthalenyl)-6-benzothiazolcarboxamide or N-(2,3-dihydro-1H-inden-2-yl)-2-(1H-pyrrol-1-yl)-6-benzothiazolecarboxamide.

In a fourth aspect of the present invention, there is provided a compound of Formula D or Formula IV

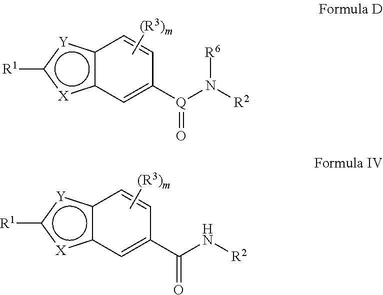

or a pharmaceutically acceptable salt or derivative thereof, wherein:
   one of X and Y is S and the other is N;
   Q is C or S(O);
   R¹ is a 4- to 10-membered monocyclic, bridged or bicyclic ring containing at least 1 ring N heteroatom and optionally a ring 0 heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring 0 heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof, wherein a straight chain portion of said (C3-8)alkyl group may be optionally interrupted by 1—O—;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
   R⁶ is H or (C1-6)alkyl; and
   m is 1,2 or 3;
   wherein, where Q is present and is S(O), R¹ is not optionally substituted pyrazol-4-yl.

Compounds described herein are shown in the Examples to activate PDE4 long form enzymes.

In a further aspect, the present invention provides a pharmaceutical composition comprising a compound or a pharmaceutically acceptable salt or derivative as described herein, and a pharmaceutically acceptable excipient.

In a further aspect, the present invention provides a compound or pharmaceutical composition described herein for use in therapy. The therapy may be the treatment or prevention of a disease or disorder that can be ameliorated by activation of long isoforms of PDE4. The therapy may be the treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Also provided is a method of treating or preventing a disease or disorder that can be ameliorated by activation of long isoforms of PDE4, comprising the step of administering an effective amount of a compound or pharmaceutical composition described herein to a patient in need thereof. Also provided is a method of treating or preventing a disease or disorder mediated by excessive intracellular cAMP signalling, comprising the step of administering an effective amount of a compound or pharmaceutical composition described herein to a patient in need thereof.

Also provided is the use of a compound or pharmaceutical composition described herein in the manufacture of a medicament for treating or preventing a disease or disorder that can be ameliorated by activation of long isoforms of PDE4. Also provided is the use of a compound or pharmaceutical composition described herein in the manufacture of a medicament for treating or preventing a disease or disorder mediated by excessive intracellular cAMP signalling.

In certain embodiments of the foregoing aspects, the compounds of the invention are provided for the treatment or prevention of a condition selected from hyperthyroidism, Jansens's metaphyseal chondrodysplasia, hyperparathyroidism, familial male-limited precocious puberty, pituitary adenomas, Cushing's disease, polycystic kidney disease, polycystic liver disease, McCune-Albright syndrome, cholera, whooping cough, anthrax, tuberculosis, HIV, AIDS, Common Variable Immunodeficiency (CVID), melanoma, pancreatic cancer, leukaemia, prostate cancer, adrenocortical tumours, testicular cancer, primary pigmented nodular adrenocortical diseases (PPNAD), Carney Complex, autosomal dominant polycystic kidney disease (ADPKD), autosomal recessive polycystic kidney disease (ARPKD), maturity onset diabetes of young type 5 (MODY5), or cardiac hypertrophy.

### BRIEF DESCRIPTION OF THE FIGURES

FIG. 1 shows dose-dependent activation of a PDE4 long form, PDE4D5, by Example 66 using the method described in Experiment 1.

FIG. 2 shows inhibition of cyst formation in a 3D culture of m-IMCD3 mouse kidney cells treated with Example 191, using the method described in Experiment 4.

FIG. 3 shows inhibition of PTH-induced cAMP elevation in the urine of anaesthetised rats treated with Example 7, using the method described in Experiment 6.

### DETAILED DESCRIPTION

The invention is based on the surprising identification of new compounds that are able to activate long isoforms of PDE4 enzymes. The compounds are small molecules and so are expected to be easier and cheaper to make and formulate into pharmaceuticals than large biological molecules such as polypeptides, proteins or antibodies. The compounds can be chemically synthesized, as demonstrated in the Examples.

The Examples demonstrate that a number of compounds of Formula A to D, Formula I to IV and Formula Z are able to activate long isoforms of PDE4. The Examples go on to demonstrate that certain tested compounds of the invention do not activate a short form of PDE4, thereby demonstrating selectivity for activation of PDE4 long forms over PDE4 short forms. The Examples further demonstrate that compounds of the present invention reduce cAMP-driven cyst formation in an in vitro model of ADPKD. The Examples also demonstrate that compounds of the present invention suppress the elevation of urinary cAMP levels by parathyroid hormone (PTH) in an in vivo model of hyperparathyroidism.

Various aspects and embodiments are disclosed herein. It will be recognised that features specified in each embodiment may be combined with other specified features to provide further embodiments.

Described herein are compounds of Formula A to D, I to IV and Z, or pharmaceutically acceptable salts or derivatives thereof. Formula A to D, I to IV and Z are illustrated herein. Compounds of Formula A to D, I to IV and Z, or pharmaceutically acceptable salts or derivatives thereof, may be provided for use in the treatment or prevention of a disease or disorder that can be ameliorated by activation of long isoforms of PDE4. Compounds of Formula A to D, I to IV and Z, or pharmaceutically acceptable salts or derivatives thereof, may be provided for use in the treatment or prevention of a disease or disorder mediated by excessive intracellular cAMP signalling.

In the compounds of Formula A and Formula I, R¹ is a 4- to 10-membered monocyclic, bridged or bicyclic ring containing at least 1 ring N heteroatom and optionally a ring O heteroatom, and wherein R¹ is optionally substituted with 1 or more R⁴. The monocyclic, bridged or bicyclic ring may be saturated, partially saturated or aromatic, or in the case of a bicyclic ring, a combination thereof. It will be appreciated that the ring N atom in a saturated or partially saturated ring, when unsubstituted, may be NH (as valency allows). It will also be appreciated that no further ring heteroatoms are present other than the “at least 1 ring N heteroatom” (i.e. 1 or more ring N heteroatoms) and the optional “ring O heteroatom”.

In embodiment (1) of Formula A or Formula I, R¹ comprises at least 1 ring N heteroatom not at the point of attachment of R¹ (i.e. a ring N atom must be present at a position that not the point of attachment of R¹ to the ring containing X and Y). The remaining moieties may be as defined for Formula A or Formula I or any of embodiments (5)-(20) of Formula A or Formula I described herein, mutatis mutandis.

In embodiment (2) of Formula A or Formula I, R¹ is a 4- to 10-membered monocyclic, bridged or bicyclic ring containing 1 ring N heteroatom, 2 ring N heteroatoms, 1 ring N heteroatom and 1 ring O heteroatom or 2 ring N heteroatoms and 1 ring O heteroatom, and wherein R¹ is optionally substituted with 1 or more R⁴. R¹ may be a 4- to 10-membered monocyclic, bridged or bicyclic ring containing 1 ring N heteroatom, 2 ring N heteroatoms or 1 ring N heteroatom and 1 ring O heteroatom, and wherein R¹ is optionally substituted with 1 or more R⁴. R¹ may comprise at least 1 ring N heteroatom not at the point of attachment of R¹. The remaining moieties may be as defined for Formula A or Formula I or any of embodiments (5)-(20) of Formula A or Formula I described herein, mutatis mutandis.

In embodiment (3) of Formula A or Formula I, R¹ is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 5- to 6-membered aromatic, monocyclic ring containing 1 or 2 ring N heteroatoms; a 7- to 8-membered saturated, bridged ring system containing 1 or 2 ring N heteroatoms or a 9-membered saturated, bridged ring system containing 2 ring N heteroatoms and a ring O-heteroatom; or a 7- to 10-membered saturated, fused or spiro ring system system containing 1 or 2 ring N heteroatoms, optionally 2 ring N heteroatoms; and R¹ is optionally substituted with 1 or more R⁴, optionally wherein R¹ is optionally substituted with 1, 2 or 3 R⁴. R¹ may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 5- to 6-membered aromatic, monocyclic ring containing 1 or 2 ring N heteroatoms; or a 7- to 8-membered saturated, bridged ring system containing 1 or 2 ring N heteroatoms; and R¹ is optionally substituted with 1 or more R⁴, optionally wherein R¹ is optionally substituted with 1, 2 or 3 R⁴. R¹ may comprise at least 1 ring N heteroatom not at the point of attachment of R¹. The remaining moieties may be as defined for Formula A or Formula I or any of embodiments (5)-(20) of Formula A or Formula I described herein, mutatis mutandis.

In Formula A or Formula I or any of the options for embodiments (1), (2) or (3), R¹ may be a 4- to 10-membered monocyclic, bridged or bicyclic ring containing at least 1 ring N heteroatom (i.e. with no ring O heteroatom). R¹ may be a 6-membered saturated or aromatic monocyclic ring containing 1 or 2 ring N heteroatoms, or a 7- to 8-membered saturated, bridged ring system containing 1 or 2 ring N heteroatoms, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and R¹ is optionally substituted with 1 or more R⁴. R¹ may be a 6-membered saturated or aromatic monocyclic ring containing 1 or 2 ring N heteroatoms, or a 7- to 8-membered saturated, bridged ring system containing 1 or 2 ring N heteroatoms, wherein at least 1 ring N heteroatom is not at the point of attachment of R¹, and wherein R¹ is optionally substituted with 1 R⁴. R¹ may be a 6-membered saturated or aromatic monocyclic ring containing 2 ring N heteroatoms; or a 7- to 8-membered saturated, bridged ring system containing 2 ring N heteroatoms, wherein R¹ is optionally substituted with 1 R⁴. R¹ may be a 7- to 8-membered saturated, bridged ring system containing 2 ring N heteroatoms, wherein R¹ is optionally substituted with 1 R⁴, R^{1a} may be a 7- to 8-membered saturated, bridged ring system containing 2 ring N heteroatoms, for example a bridged piperazine, such as 3,8-diazabicyclo[3.2.1]octanyl, wherein R^{1a} is optionally substituted with 1 R⁴.

In Formula A or Formula I or any of the options for embodiments (1), (2) or (3), R¹ may be piperidinyl, piperazinyl, morpholinyl, pyrrolidinyl, pyrazolyl, imidazolyl, pyridinyl, azetidinyl, 2,5-diazabicyclo[2.2.1]heptanyl, 2,5-diazabicyclo[2.2.2]octanyl, 3,8-diazabicyclo[3.2.1]octanyl, 3,6-diazabicyclo[3.1.1]heptanyl, 4,7-diazaspiro[2.5]octanyl, 2,6-diazaspiro[3.3]heptanyl, 2,6-diazaspiro[3.4]octanyl, 2,7-diazaspiro[3.5]nonanyl, octahydro-4H-pyrrolo[3,2-b]pyridinyl, octahydro-5H-pyrrolo[3,2-c]pyridinyl or hexahydropyrrolo[3,4-c]pyrrol-(1H)-yl each of which is optionally substituted with 1 or more R⁴, preferably optionally substituted with 1-3 R⁴, preferably optionally substituted with 1 R⁴. R¹ may be piperidinyl, piperazinyl, morpholinyl, pyrrolidinyl, pyrazolyl, imidazolyl, pyridinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be a group of structure:

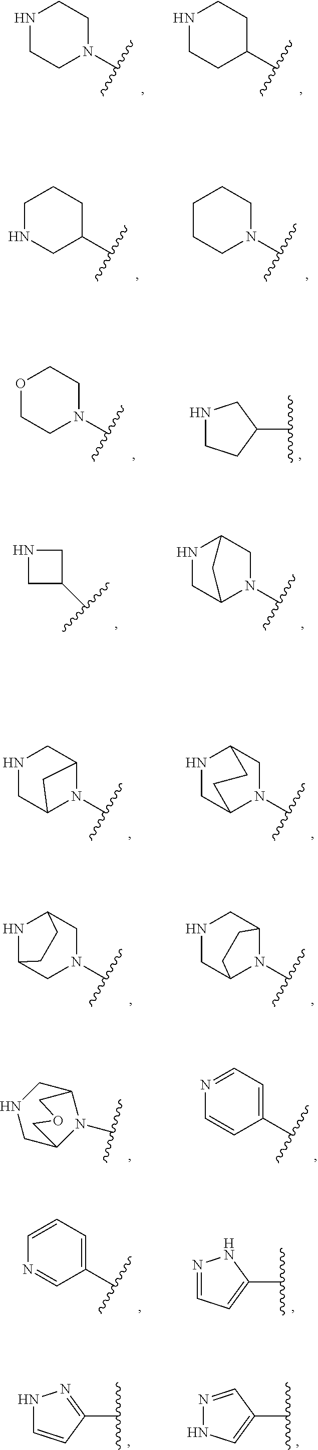

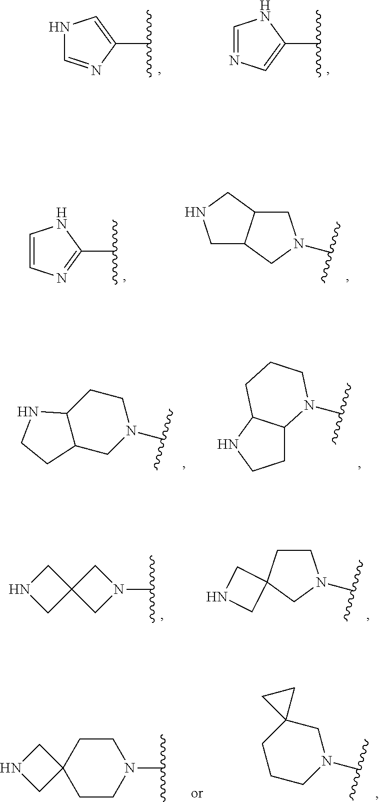

and wherein R¹ is optionally substituted with 1 or more R⁴, optionally wherein R¹ is optionally substituted with 1-3 R⁴. R¹ may be a group of structure:

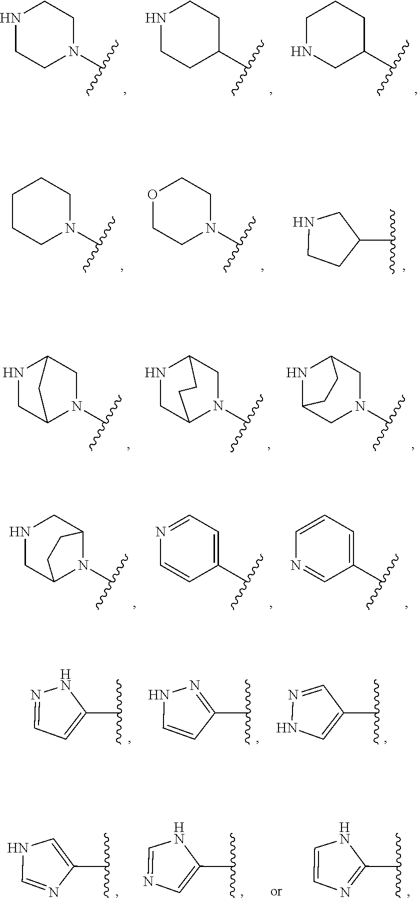

and wherein R¹ is optionally substituted with 1 or more R⁴, optionally wherein R¹ is optionally substituted with 1-3 R⁴. R¹ may be piperidinyl, piperazinyl, pyrrolidinyl, pyrazolyl, imidazolyl, pyridinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R¹ may be piperidinyl, piperazinyl or pyridinyl, each of which is optionally substituted with 1 or more R⁴, preferably optionally substituted with 1-3 R⁴, preferably optionally substituted with 1 R⁴. R¹ may be: a group of structure

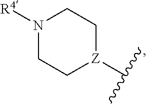

wherein Z is CH or N and R^{4′} is H or R⁴; or pyridyl (optionally 3-pyridyl) optionally substituted with 1 R⁴. R¹ may be a 7- to 8-membered saturated, bridged ring system containing 2 ring N heteroatoms, for example a bridged piperazine such as

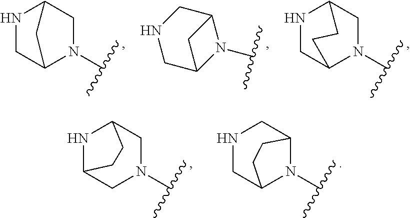 [SMILES: *N1CC2CC1CN2 ; *N1C2CNCC1C2 ; *N1CC2CCC1CN2 ; *N1CC2CCC(C1)N2 ; *N1C2CCC1CNC2]

The remaining moieties may be as defined for Formula A or Formula I or any of embodiments (5)-(20) of Formula A or Formula I described herein, mutatis mutandis.

In embodiment (4) of Formula A or Formula I, R¹ is a 5- to 6-membered saturated, monocyclic ring containing at least 1 ring N heteroatom and optionally a ring O heteroatom; or a 7- to 8-membered saturated, bridged ring system containing 1 or 2 ring N heteroatoms, and wherein R¹ is optionally substituted with 1, 2 or 3 R⁴. In any of the options for embodiment (4), R¹ may comprise at least 1 ring N heteroatom not at the point of attachment of R¹. R¹ may be a 6-membered saturated monocyclic ring containing 1 or 2 ring N heteroatoms, or a 7- to 8-membered saturated, bridged ring system containing 1 or 2 ring N heteroatoms. R¹ may be a 6-membered saturated monocyclic ring containing 1 or 2 ring N heteroatoms, or a 7- to 8-membered saturated, bridged ring system containing 1 or 2 ring N heteroatoms, wherein R¹ is optionally substituted with 1 R⁴. R¹ may be a 7- to 8-membered saturated, bridged ring system containing 1 or 2 ring N heteroatoms, wherein R¹ is optionally substituted with 1 or more R⁴. R¹ may be a 7- to 8-membered saturated, bridged ring system containing 1 or 2 ring N heteroatoms, wherein R¹ is optionally substituted with 1 R⁴. The remaining moieties may be as defined for Formula A or Formula I or any of embodiments (5)-(20) of Formula A or Formula I described herein, mutatis mutandis.

In Formula A or Formula I or any of the options for embodiments (1)-(4), R¹ may be substituted with 1 or more R⁴. Where R¹ contains a substitutable ring N atom, R¹ may be substituted on a substitutable ring N atom. In embodiments where R¹ is saturated ring, R¹ may be substituted by 1 R⁴, preferably on a ring N atom. In embodiments where R¹ is an aromatic ring, R¹ may be substituted by 1, 2 or 3 R⁴. In embodiments where R¹ is a 6-membered ring, R¹ may be substituted by 1 R⁴, for example where R¹ is a bridged 6-membered ring, R¹ may be substituted by 1 R⁴. In embodiments where R¹ is a 5-membered ring, R¹ may be substituted by 1,2 or 3 R⁴.

In the compounds of Formula A and Formula I, each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may, independently, represent a substituent on a carbon atom or a substitutable N atom.

In embodiment (5) of Formula A or Formula I, each R⁴ is independently halogen, OH, CN, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, CN, (C1-4)alkyl, methoxy, ethoxy, cyclopropyl or —(CH₂)2-O—(CH₂)₂O—CH₃, the (C1-4)alkyl being optionally substituted with 1 or more substituents independently selected from halogen and OH. The remaining moieties may be as defined for Formula A or Formula I or any of embodiments (1)-(4) or (8)-(20) of Formula A or Formula I described herein, mutatis mutandis.

In embodiment (6) of Formula A or Formula I, each R⁴ is independently halogen, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy. Each R⁴ may independently be halogen, OH, (C1-4)alkyl, (C1-3)alkoxy, (C3-6)cycloalkyl or —(C1-3)alkylene-(C1-3)alkoxy, the (C1-3)alkyl, (C1-3)alkoxy, (C3-6)cycloalkyl and —(C1-3)alkylene-(C1-3)alkoxy being optionally substituted with 1 or more substituents independently selected from halogen, OH and (C1-3)alkoxy. Each R⁴ may independently be F, Cl, OH, (C1-4)alkyl, methoxy, ethoxy, cyclopropyl or —(CH₂)₂—O—(CH₂)₂O—CH₃, the (C1-4)alkyl being optionally substituted with 1 or more substituents independently selected from halogen and OH. The remaining moieties may be as defined for Formula A or Formula I or any of embodiments (1)-(4) or (8)-(20) of Formula A or Formula I described herein, mutatis mutandis.

In embodiment (7) of Formula A or Formula I, each R⁴ is independently halogen, CN, OH, (C1-2)alkyl, (C1-6)alkoxy, or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy. Each R⁴ may independently be F, Cl, OH, (C1-2)alkyl, methoxy, ethoxy or —(CH₂)₂—O—(CH₂)₂O—CH₃, the (C1-2)alkyl being optionally substituted with 1 or more substituents independently selected from halogen and OH. The remaining moieties may be as defined for Formula A or Formula I or any of embodiments (1)-(4) or (8)-(20) of Formula A or Formula I described herein, mutatis mutandis.

In Formula A or Formula I or any of the options for embodiments (5)-(7), when attached to a ring N atom, R⁴ may independently be any of the options identified herein for R⁴, except for halogen, CN, OH, and —(C1-6)alkoxy.

In the compounds of Formula A and Formula I, R² is (C5-7)cycloalkyl, fused to a 6-membered aromatic or heteroaromatic ring that contains 0, 1 or 2 ring N atoms; a 5- to 7-membered non-aromatic heterocycle containing one ring 0 heteroatom, optionally fused to a 6-membered aromatic or heteroaromatic ring that contains 0, 1 or 2 ring N atoms; CH₂Ar, where Ar is a 6-membered aromatic or heteroaromatic ring that contains 0, 1 or 2 ring N atoms; or (C3-8)alkyl group that may be straight chain, branched or cyclic, or a combination thereof, wherein a straight chain portion of said (C3-8)alkyl group may be optionally interrupted by 1—O—; wherein R² is optionally substituted with 1 or more R⁵. It will be appreciated that where an alkyl group is “interrupted” by a heteroatom such as 0, the heteroatom is present in addition to the number of carbon atoms of the alkyl group. R² may be (C5-7)cycloalkyl, fused to a 6-membered aromatic or heteroaromatic ring that contains 0, 1 or 2 ring N atoms; a 5- to 7-membered non-aromatic heterocycle containing one ring 0 heteroatom, optionally fused to a 6-membered aromatic or heteroaromatic ring that contains 0, 1 or 2 ring N atoms; CH₂Ar, where Ar is a 6-membered aromatic or heteroaromatic ring that contains 0, 1 or 2 ring N atoms; or (C3-8)alkyl group that may be straight chain, branched or cyclic, or a combination thereof; wherein R² is optionally substituted with 1 or more R⁵.

In the compounds of Formula A and Formula I, each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH.

In embodiment (8) of Formula A or Formula I, each R⁵ is independently halogen, OH, CN, (C1-4)alkyl, or (C1-4)alkoxy, the (C1-4)alkyl and (C1-4)alkoxy group being optionally substituted with 1 or more halogen or OH, preferably optionally substituted with 1 or more fluoro or 1 OH. The remaining moieties may be as defined for Formula A or Formula I or any of embodiments (1)-(7) or (9)-(20) of Formula A or Formula I described herein, mutatis mutandis.

In embodiment (9) of Formula A or Formula I, R² is (C5-7)cycloalkyl, fused to a 6-membered aromatic or heteroaromatic ring that contains 0, 1 or 2 ring N atoms, wherein R² is optionally substituted with 1 or more R⁵. R² may be (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R² may be indane optionally substituted with 1 to 3 R⁵, preferably 1 R⁵. The remaining moieties may be as defined for Formula A or Formula I or any of embodiments (1)-(8) or (17)-(20) of Formula A or Formula I described herein, mutatis mutandis. In any of the options for embodiment (9), R² may be optionally substituted with 1 instance of halogen, OH, CN, (C1-4)alkyl or (C1-4)alkoxy.

In embodiment (10) of Formula A or Formula I, R² is a 5- to 7-membered non-aromatic heterocycle containing one ring O heteroatom, optionally fused to a 6-membered aromatic or heteroaromatic ring that contains 0, 1 or 2 ring N atoms, wherein R² is optionally substituted with 1 or more R⁵. R² may be a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R² may be chromane or tetrahydropyran optionally substituted with 1 to 3 R⁵, preferably 1 R⁵. The remaining moieties may be as defined for Formula A or Formula I or any of embodiments (1)-(8) or (17)-(20) of Formula A or Formula I described herein, mutatis mutandis. In any of the options for embodiment (10), R² may be optionally substituted with 1 instance of halogen, OH, CN, (C1-4)alkyl or (C1-4)alkoxy. The remaining moieties may be as defined for any aspect or embodiment of Formula A or Formula I described herein, mutatis mutandis.

In embodiment (11) of Formula A or Formula I, R² is CH₂Ar, where Ar is a 6-membered aromatic or heteroaromatic ring that contains 0, 1 or 2 ring N atoms, wherein R² is optionally substituted with 1 or more R⁵. It will be appreciated that substitution by R⁵ is possible on the —CH₂— linker or Ar moiety of R². R² may be CH₂Ar, wherein the Ar is optionally substituted with 1 to 3 substituents selected from halogen, CN, (C1-4)alkyl, (C1-4)alkoxy and the CH₂ is optionally substituted with (C1-4)alkyl or —(C1-6)alkylene-(C1-6)alkoxy, the (C1-4)alkyl group being optionally substituted with OH. R² may be benzyl optionally substituted with 1 to 3 R⁵, preferably 1 R⁵. The remaining moieties may be as defined for Formula A or Formula I or any of embodiments (1)-(8) or (17)-(20) of Formula A or Formula I described herein, mutatis mutandis. In any of the options for embodiment (11), R² may be optionally substituted with 1 instance of halogen, OH, CN, (C1-4)alkyl or (C1-4)alkoxy, the (C1-4)alkyl group being optionally substituted with OH.

In embodiment (12) of Formula A or Formula I, R² is a (C3-8)alkyl group that may be straight chain, branched or cyclic, or a combination thereof, wherein a straight chain portion of said (C3-8)alkyl group may be optionally interrupted by 1-O—, wherein R² is optionally substituted with 1 or more R⁵. R² may be a (C3-8)alkyl group that may be straight chain, branched or cyclic, or a combination thereof, wherein R² is optionally substituted with 1 or more R⁵. R² may be a (C4-8)alkyl group that may be straight chain, branched or cyclic, or a combination thereof, wherein a straight chain portion of said (C4-8)alkyl group may be optionally interrupted by 1-O—, wherein R² is optionally substituted with 1 or more R⁵. R² may be an optionally substituted (C3-6)alkyl group that may be branched or cyclic. R² may be an optionally substituted (C4-6)alkyl group that may be branched or cyclic. R² may be an optionally substituted (C4-6)cycloalkyl group, preferably an optionally substituted (C5-6)cycloalkyl group. R² may be cyclohexyl, cyclopentyl, cyclobutyl or isopropyl optionally substituted with 1 to 3 R⁵, preferably 1 R⁵. R² may be cyclohexyl, cyclopentyl or cyclobutyl optionally substituted with 1 to 3 R⁵. R² may be (C4-6)cycloalkyl substituted with 2 or more R⁵. The remaining moieties may be as defined for Formula A or Formula I or any of embodiments (1)-(8) or (17)-(20) of Formula A or Formula I described herein, mutatis mutandis. In any of the options for embodiment (12), R² may be optionally substituted with 1 or more halogen, (C1-4)alkoxy or OH. R² may be optionally substituted with 1 or 2 instances of halogen or OH. R² may be optionally substituted with 1 OH. R² may be optionally substituted with 2 or 3 instances of fluoro, preferably 2 instances of fluoro on the same carbon atom. In any of the options for embodiment (12), R² may be substituted by 2 or 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro. R² may be a (C5-6)cycloalkyl group substituted by 2 halogen substituents (optionally on a single ring carbon atom).

In embodiment (13) of Formula A or Formula I, R² is as defined in embodiment (9), embodiment (10) or embodiment (12) of Formula A or Formula I. R² may be (C5-6)cycloalkyl fused to a phenyl ring; a 5- to 6-membered non-aromatic heterocycle containing one ring O heteroatom, optionally fused to a phenyl ring; or (C4-6)cycloalkyl; wherein R² is optionally substituted with 1 or more R⁵. R² may be a group of formula

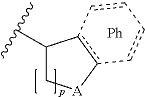

wherein A is O or CH₂; p is 1 or 2; Ph is an optionally present, fused phenyl ring, and wherein R² is optionally substituted with 1 or more R⁵ (for example, 1 or 2 R⁵); optionally wherein when A is O, p is 2 or when A is CH₂, p is 1 or 2. A may be O or C(R⁵)₂ (i.e CH₂, with two R⁵ substituents, for example, CF₂). Ph may be absent. The remaining moieties may be as defined for Formula A or Formula I or any of embodiments (1)-(8) or (17)-(20) of Formula A or Formula I described herein, mutatis mutandis.

In an embodiment (14) of Formula A or Formula I, R² is a group of formula

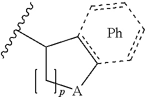

wherein A is O or CH₂; p is 1, 2 or 3; Ph is an optionally present, fused phenyl ring, and wherein R² is optionally substituted with 1 or more R⁵ (for example, 1 or 2 R⁵); optionally wherein when A is O, p is 2 or when A is CH₂, p is 1 or 2. A may be O or C(R⁵)₂ (for example, CF₂). When A is CH₂, Ph may preferably be present. The remaining moieties may be as defined for Formula A or Formula I or any of embodiments (1)-(8) or (17)-(20) of Formula A or Formula I described herein, mutatis mutandis.

In embodiment (15) of Formula A or Formula I, R² is as defined in embodiment (9), embodiment (10) or embodiment (11) of Formula A or Formula I. The remaining moieties may be as defined for Formula A or Formula I or any of embodiments (1)-(8) or (17)-(20) of Formula A or Formula I described herein, mutatis mutandis.

In embodiment (16) of Formula A or Formula I, R² is as defined in embodiment (10), embodiment (11) or embodiment (12) of Formula A or Formula I. The remaining moieties may be as defined for Formula A or Formula I or any of embodiments (1)-(8) or (17)-(20) of Formula A or Formula I described herein, mutatis mutandis.

In Formula A or Formula I or any of the options for embodiments (9)-(16), R² may be substituted with 1 or more R⁵, preferably 1, 2 or 3 R⁵. R² may be substituted with 1 R⁵. R² may be substituted with 2 R⁵. It will be appreciated that each R⁵ substituent may be present on the same atom or on a different atom, as allowed by valency.

In the compounds of Formula A and Formula I, each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy, optionally each R³ is independently (C1-6)alkyl, (C1-6)alkoxy, CN or halogen, the (C1-6)alkyl and (C1-6)alkoxy being optionally substituted by 1 or more halogen.

In embodiment (17) of Formula A or Formula I, each R³ is independently (C1-4)alkyl, (C1-4)alkoxy, CN or halogen, the (C1-4)alkyl and (C1-4)alkoxy being optionally substituted by 1 or more halogen, OH or (C1-4)alkoxy. Each R³ may independently be (C1-4)alkyl, (C1-4)alkoxy, CN or halogen, the (C1-4)alkyl and (C1-4)alkoxy being optionally substituted by 1 or more halogen. Each R³ may independently be —CH₃,—OCH₃, CN, halogen, cyclopropyl or (C1-3)alkyl substituted with OH. Each R³ may independently be —CH₃,—OCH₃, CN or halogen. Each R³ may independently be —CH₃ or —OCH₃. The remaining moieties may be as defined for Formula A or Formula I or any of embodiments (1)-(16) or (18)-(20) of Formula A or Formula I described herein, mutatis mutandis.

In the compounds of Formula A and Formula I, n is 0, 1, 2 or 3.

In embodiment (18) of Formula A or Formula I, n is 0, 1 or 2. n may be 0 or 1. The remaining moieties may be as defined for Formula A or Formula I or any of embodiments (1)-(17) or (20) of Formula A or Formula I described herein, mutatis mutandis.

In embodiment (19) of Formula A or Formula I, n is 1, 2 or 3. n may be 1. The remaining moieties may be as defined for Formula A or Formula I or any of embodiments (1)-(17) or (20) of Formula A or Formula I described herein, mutatis mutandis.

In the compounds of Formula A and Formula I, one of X and Y is S and the other is N.

In embodiment (20) of Formula A or Formula I, X is S and Y is N. The remaining moieties may be as defined for Formula A or Formula I or any of embodiments (1)-(19) of Formula A or Formula I described herein, mutatis mutandis.

In the compounds of Formula A, Q is C or S(O).

In the compounds of Formula A, R⁶ is H or (C1-6)alkyl (for example, (C1-3)alkyl such as methyl).

In embodiment (21) of Formula A or Formula I,
X is S and Y is N;
R¹ is a 6-membered saturated or aromatic monocyclic ring containing 1 or 2 (optionally 2) ring N heteroatoms or a 7- to 8-membered saturated, bridged ring system containing 1 or 2 (optionally 2) ring N heteroatoms, wherein at least 1 ring N heteroatom is not at the point of attachment of R¹, and wherein R¹ is optionally substituted with 1 R⁴, optionally R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴;
R² is
(i) (C5-6)cycloalkyl, optionally fused to a phenyl ring; or
(ii) a 5- to 6-membered non-aromatic heterocycle containing one O heteroatom, optionally fused to a phenyl ring;
and wherein R² is optionally substituted with 1 or 2 R⁵;
R³, where present, is methyl, CN or halogen, optionally methyl;
R⁴, where present, is (C1-6)alkyl optionally substituted with OH, optionally (C1-2)alkyl optionally substituted with OH;
R⁵, where present, is OH or halo, optionally OH; and
n is 0 or 1.

In embodiment (21) of Formula A or Formula I, R² may be a group of formula

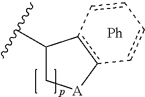

wherein A is O or CH₂; p is 1 or 2; Ph is an optionally present, fused phenyl ring, and wherein R² is optionally substituted with 1 or 2 R⁵; optionally wherein when A is O, p is 2 or when A is CH₂, p is 1 or 2. A may be O or C(R⁵)₂ (i.e CH₂, with two R⁵ substituents, for example, CF₂). Ph may be absent.

In embodiment (21) of Formula A or Formula I, R² may be a group of formula

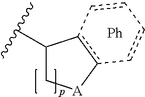

wherein A is O or CH₂; p is 1, 2 or 3 (optionally 1 or 2); Ph is an optionally present, fused phenyl ring, and wherein R² is optionally substituted with 1 or 2 R⁵; optionally wherein when A is O, p is 2 or when A is CH₂, p is 1 or 2. A may be O or C(R⁵)₂ (i.e CH₂, with two R⁵ substituents, for example, CF₂). When A is CH₂, Ph may preferably be present.

Embodiments (1) to (4) of Formula A or Formula I may apply to any of the options for embodiment (21) of Formula A or Formula I, mutatis mutandis.

In embodiment (22) of Formula A or Formula I, R¹ is according to embodiment (4) of Formula A or Formula I and R² is according to embodiment (13) of Formula A or Formula I. The remaining moieties may be as defined for Formula A or Formula I or any of embodiments (1)-(20) of Formula A or Formula I described herein, mutatis mutandis. R¹ may be a 7- to 8-membered saturated, bridged ring system containing 1 or 2 ring N heteroatoms, wherein R¹ is optionally substituted with 1 R⁴; and R² may be according to embodiment (13) of Formula A or Formula I. R¹ may be a 7- to 8-membered saturated, bridged ring system containing 1 or 2 ring N heteroatoms, wherein R¹ is optionally substituted with 1 R⁴; and R² may be (C4-6)cycloalkyl substituted with 1 or more R⁵. R¹ may be a 7- to 8-membered saturated, bridged ring system containing 1 or 2 ring N heteroatoms, wherein R¹ is optionally substituted with 1 R⁴; and R² may be (C4-6)cycloalkyl substituted with 2 or more R⁵; optionally wherein R⁵ may be halogen; and n may be 0 or 1.

In embodiment (23) of Formula A or Formula I, when R¹ is 4-cyclopentylpiperazin-1-yl, 4-cyclopropylpiperazin-1-yl or 4-isopropylpiperazin-1-yl, Q when present is C, and n is 0, R² is not unsubstituted, uninterrupted, straight chain or branched (C3-6)alkyl or unsubstituted (C3-8)cycloalkyl. The remaining moieties may be as defined for Formula A or Formula I or any of embodiments (1)-(20) of Formula A or Formula I described herein, mutatis mutandis.

In embodiment (24) of Formula A or Formula I, when R¹ is 1-piperazinyl, R² is not a straight chain, branched chain or cyclic (C3)alkyl group. The remaining moieties may be as defined for Formula A or Formula I or any of embodiments (1)-(20) of Formula A or Formula I described herein, mutatis mutandis. In some embodiments, the compound is not 2-(1-piperazinyl)-N-propyl-6-benzothiazolecarboxamide, N-(1-methylethyl)-2-(1-piperazinyl)-6-benzothiazolecarboxamide or N-cyclopropyl-2-(1-piperazinyl)-6-benzothiazolecarboxamide.

In embodiment (25) of Formula A or Formula I, when R¹ is 4-morpholinyl, R² is not 1,2,3,4-tetrahydro-1-naphthalenyl. The remaining moieties may be as defined for Formula A or Formula I or any of embodiments (1)-(20) of Formula A or Formula I described herein, mutatis mutandis. In some embodiments, the compound is not 2-(4-morpholinyl)-N-(1,2,3,4-tetrahydronaphthalenyl)-6-benzothiazolcarboxamide.

In embodiment (26) of Formula A or Formula I, when Q is present and is S(O), R¹ is not optionally substituted pyrazol-4-yl, e.g. optionally substituted

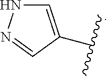 [SMILES: *c1cn[nH]c1]

The remaining moieties may be as defined for Formula A or Formula I or any of embodiments (1)-(20) of Formula A or Formula I described herein, mutatis mutandis.

In embodiment (27) of Formula A or Formula I, R¹ is not pyrrol-1-yl. The remaining moieties may be as defined for Formula A or Formula I or any of embodiments (1)-(20) of Formula A or Formula I described herein, mutatis mutandis. In some embodiments, the compound is not N-(2,3-dihydro-1H-inden-2-yl)-2-(1H-pyrrol-1-yl)-6-benzothiazolecarboxamide.

It will be appreciated that compounds of Formula A where Q is C and R⁶ is H correspond to the compounds of Formula I.

Compounds of Formula A and Formula I include compounds of Formulas B-D and II-IV. Embodiments (1)-(27) of Formula A or Formula I may apply mutatis mutandis to each of Formulas B-D and II-IV.

Described herein is a compound of Formula B or Formula II

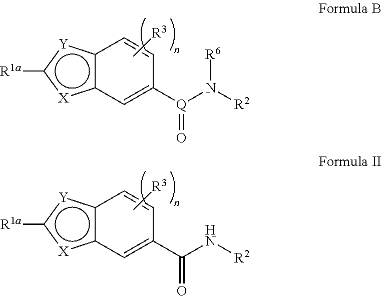

or a pharmaceutically acceptable salt or derivative thereof, wherein:
R^{1a} is a 4- to 10-membered non-aromatic, monocyclic, bridged or bicyclic ring containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
and X, Y, Q, R², R³, R⁴, R⁵, R⁶ and n are as defined for Formula A or Formula I or any of embodiments (5)-(20) of Formula A or Formula I above. In a compound of Formula B or Formula II, R^{1a} comprises at least one ring N heteroatom not at the point of attachment to R^{1a}, i.e. a ring N atom must be present at a position that is not the point of attachment of R^{1a} to the ring containing X and Y.

In embodiment (1) of Formula B or Formula II, when R^{1a} is 4-cyclopentylpiperazin-1-yl, 4-cyclopropylpiperazin-1-yl or 4-isopropylpiperazin-1-yl, Q if present is C and n is 0, R² is not unsubstituted, straight chain or branched (C3-6)alkyl or unsubstituted (C3-8)cycloalkyl.

In embodiment (2) of Formula B or Formula II:
(i) each R⁴ is independently halogen, CN, OH, (C1-2)alkyl, (C1-6)alkoxy or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy; and/or
(ii) n is 1, 2 or 3; and/or
(iii) R² is (C5-7)cycloalkyl, fused to a 6-membered aromatic or heteroaromatic ring that contains 0, 1 or 2 ring N atoms; a 5- to 7-membered non-aromatic heterocycle containing one ring O heteroatom, optionally fused to a 6-membered aromatic or heteroaromatic ring that contains 0, 1 or 2 ring N atoms; or CH₂Ar, where Ar is a 6-membered aromatic or heteroaromatic ring that contains 0, 1 or 2 ring N atoms; and wherein R² is optionally substituted with 1 or more R⁵.

In embodiment (3) of Formula B or Formula II, R^{1a} is a 4- to 10-membered non-aromatic, monocyclic, bridged or bicyclic ring containing 1 ring N heteroatom, 2 ring N heteroatoms or 1 ring N heteroatom and 1 ring O heteroatom or 2 ring N heteroatoms and 1 ring O heteroatom, and wherein R^{1a} is optionally substituted with 1 or more R⁴. R^{1a} may be a 4- to 10-membered non-aromatic, monocyclic, bridged or bicyclic ring containing 1 ring N heteroatom, 2 ring N heteroatoms or 1 ring N heteroatom and 1 ring O heteroatom, and wherein R^{1a} is optionally substituted with 1 or more R⁴. R^{1a} may be a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); a 7- to 8-membered saturated, bridged ring system containing 1 or 2 ring N heteroatoms; or a 9-membered saturated, bridged ring system containing 2 ring N heteroatoms and a ring O-heteroatom; or a 7- to 10-membered saturated, fused or spiro ring system containing 1 or 2 ring N heteroatoms, and wherein R^{1a} is optionally substituted with 1 or more R⁴, optionally 1, 2 or 3 R⁴.

In embodiment (4) of Formula B or Formula II, R^{1a} is a 5- to 6-membered saturated, monocyclic ring containing at least 1 ring N heteroatom and optionally a ring O heteroatom (for example, 1 ring N heteroatom, 2 ring N heteroatoms or 1 ring N heteroatom and 1 ring O heteroatom); or a 7- to 8-membered saturated, bridged ring system containing 1 or 2 ring N heteroatoms, and wherein R^{1a} is optionally substituted with 1 or more R⁴, optionally 1, 2 or 3 R⁴. R^{1a} may be a 6-membered saturated monocyclic ring containing 1 or 2 ring N heteroatoms, optionally wherein at least 1 ring N heteroatom is not at the point of attachment of R^{1a}. R^{1a} may be a 6-membered saturated monocyclic ring containing 1 or 2 ring N heteroatoms, wherein R¹¹ is optionally substituted with 1 R⁴. R^{1a} may be a 7- to 8-membered saturated, bridged ring system containing 1 or 2 ring N heteroatoms, and wherein R¹ is optionally substituted with 1 or more R⁴, optionally 1, 2 or 3 R⁴. R^{1a} may be a 7- to 8-membered saturated, bridged ring system containing 2 ring N heteroatoms, for example a bridged piperazine, such as 3,8-diazabicyclo[3.2.1]octanyl, wherein R¹ is optionally substituted with 1 R⁴.

In Formula B or Formula II or any of the options for embodiments (3) or (4), R^{1a} may be piperidinyl, piperazinyl, morpholinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1a} may be group of structure:

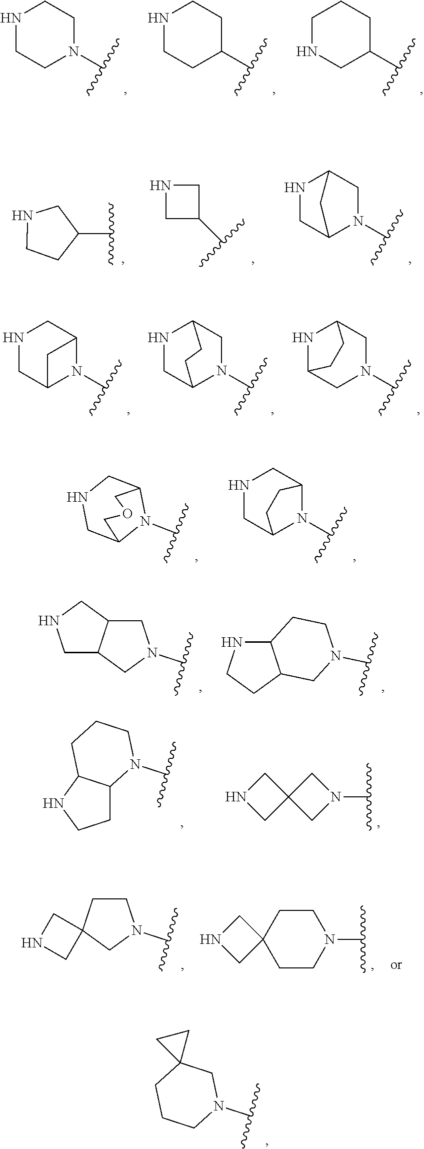

and wherein R^{1a} is optionally substituted with 1 or more R⁴, optionally wherein R^{1a} is optionally substituted with 1-3 R⁴, preferably optionally substituted with 1 R⁴. R^{1a} may be piperidinyl, piperazinyl, pyrrolidinyl, 2,5-diazabicyclo[2.2.1]heptanyl, 2,5-diazabicyclo[2.2.2]octanyl or 3,8-diazabicyclo[3.2.1]octanyl, each of which is optionally substituted with 1 or more R⁴, preferably optionally substituted with 1-3 R⁴, preferably optionally substituted with 1 R⁴. R^{1a} may be piperidinyl or piperazinyl, each of which is optionally substituted with 1 or more R⁴, preferably optionally substituted with 1-3 R⁴, preferably optionally substituted with 1 R⁴. R^{1a} may be a group of structure

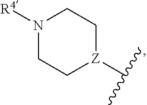

wherein Z is CH or N and R^{4′} is H or R⁴. R^{1a} may be a 7- to 8-membered saturated, bridged ring system containing 2 ring N heteroatoms, for example a bridged piperazine such as

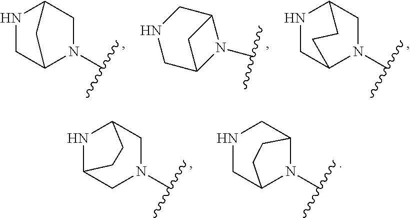 [SMILES: *N1CC2CC1CN2 ; *N1C2CNCC1C2 ; *N1CC2CCC1CN2 ; *N1CC2CCC(C1)N2 ; *N1C2CCC1CNC2]

In Formula B or Formula II or any of the options for embodiment (3) or (4) of Formula B or Formula II, R^{1a} may be optionally substituted with 1 or more R⁴. Where R^{1a} contains a substitutable ring N atom R^{1a} may preferably be substituted on a substitutable ring N atom. R^{1a} may be substituted by 1 R⁴, preferably on a ring N atom.

In Formula B or Formula II or any of the options for embodiments (3) or (4) of Formula B or Formula II, R^{1a} may be a 4- to 10-membered non-aromatic, monocyclic, bridged or bicyclic ring containing at least 1 ring N heteroatom (i.e. with no ring O heteroatom). R^{1a} may be a 6-membered saturated monocyclic ring containing 1 or 2 ring N heteroatoms, or a 7- to 8-membered saturated, bridged ring system containing 1 or 2 ring N heteroatoms, and R^{1a} is optionally substituted with 1 or more R⁴.

In embodiment (5) of Formula B or Formula II, the compound is a compound of Formula B′ or B″ or Formula IIa or IIb:

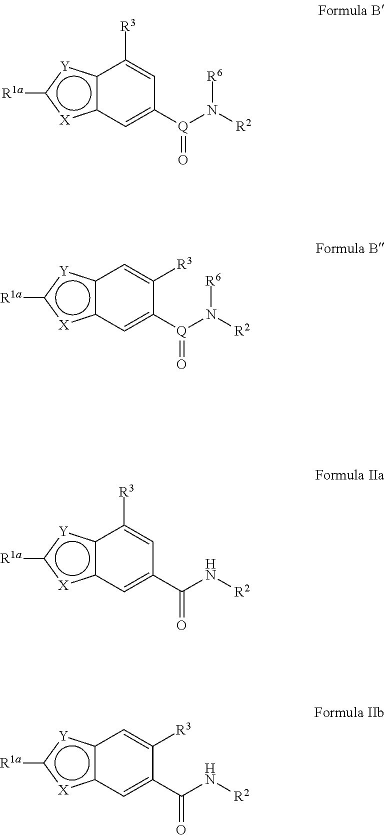

or a pharmaceutically acceptable salt or derivative thereof. R^{1a} may be as defined in embodiment (3) or embodiment (4) of Formula B or Formula II.

Embodiments (1) and (2) of Formula B or Formula II may apply to any of embodiments (3)-(5) of Formula B or Formula II, mutatis mutandis.

In embodiment (6) of Formula B or Formula II,
X is S and Y is N;
R^{1a} is a 6-membered saturated monocyclic ring containing 1 or 2 ring (optionally 2) N heteroatoms or a 7- to 8-membered saturated, bridged ring system containing 1 or 2 (optionally 2) ring N heteroatoms, wherein at least 1 ring N heteroatom is not at the point of attachment of R^{1a}, and wherein R^{1a} is optionally substituted with 1 R⁴, optionally R^{1a} is a 6-membered saturated monocyclic ring containing 1 or 2 ring N heteroatoms, wherein at least 1 ring N heteroatom is not at the point of attachment of R^{1a}, and wherein R^{1a} is optionally substituted with 1 R⁴;
R² is
(i) (C5-6)cycloalkyl, optionally fused to a phenyl ring; or
(ii) a 5- to 6-membered non-aromatic heterocycle containing one O heteroatom, optionally fused to a phenyl ring;
and wherein R² is optionally substituted with 1 or 2 R⁵;
R³, where present, is methyl, CN or halogen, optionally methyl;
R⁴, where present, is (C1-6)alkyl optionally substituted with OH, optionally (C1-2)alkyl optionally substituted with OH;
R⁵, where present, is OH or halo, optionally OH; and
n is 0 or 1.

In embodiment (6) of Formula B or Formula II, R² may be a group of formula

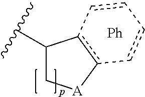

wherein A is O or CH₂; p is 1 or 2; Ph is an optionally present, fused phenyl ring, and wherein R² is optionally substituted with 1 or 2 R⁵; optionally wherein when A is O, p is 2 or when A is CH₂, p is 1 or 2. A may be O or C(R⁵)₂ (i.e CH₂, with two R⁵ substituents, for example, CF₂). Ph may be absent.

Embodiment (1) of Formula B or Formula II may apply to embodiment (6) of Formula B or Formula II, mutatis mutandis.

In embodiment (7) of Formula B or Formula II, R^{1a} is according to embodiment (4) of Formula B or Formula II and R² is according to embodiment (13) of Formula A or Formula I. The remaining moieties may be as defined for Formula B or Formula II or any of embodiments of Formula B or Formula II described herein, mutatis mutandis. R^{1a} may be a 7- to 8-membered saturated, bridged ring system containing 1 or 2 ring N heteroatoms, wherein R^{1a} is optionally substituted with 1 R⁴; and R² may be according to embodiment (13) of Formula A or Formula I. R^{1a} may be a 7- to 8-membered saturated, bridged ring system containing 1 or 2 ring N heteroatoms, wherein R^{1a} is optionally substituted with 1 R⁴; and R² may be (C4-6)cycloalkyl substituted with 1 or more R⁵. R^{1a} may be a 7- to 8-membered saturated, bridged ring system containing 1 or 2 ring N heteroatoms, wherein R^{1a} is optionally substituted with 1 R⁴; and R² may be (C4-6)cycloalkyl substituted with 2 or more R⁵; optionally wherein R⁵ is halogen; and n is 0 or 1.

In embodiment (8) of Formula B or Formula II, when R^{1a} is 1-piperazinyl, R² is not a straight chain, branched chain or cyclic (C3)alkyl group. The remaining moieties may be as defined for Formula B or Formula II or embodiments of Formula B or Formula II described herein, mutatis mutandis. In some embodiments, the compound is not 2-(1-piperazinyl)-N-propyl-6-benzothiazolecarboxamide, N-(1-methylethyl)-2-(1-piperazinyl)-6-benzothiazolecarboxamide or N-cyclopropyl-2-(1-piperazinyl)-6-benzothiazolecarboxamide.

Described herein is a compound of Formula C or a compound of Formula III

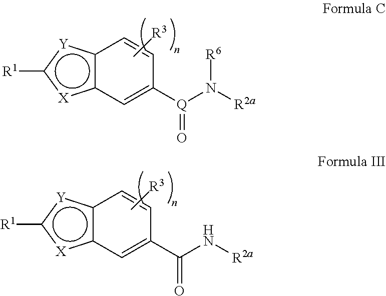

or a pharmaceutically acceptable salt or derivative thereof, wherein: R^{2a} is
(i) (C5-7)cycloalkyl fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms; or
(iv) a (C4-6)cycloalkyl group, optionally a (C5-6)cycloalkyl group;
and wherein R^{2a} is optionally substituted with 1 or more R⁵;
and X, Y, Q, R¹, R³, R⁴, R⁵, R⁶ and n are as defined for Formula A or Formula I or any of embodiments (1)-(8) or (17)-(20) of Formula A or Formula I above; and
wherein when R^{2a} is (iv) a (C4-6)cycloalkyl group it is substituted by at least 2 R⁵.

In embodiment (1) of Formula C or Formula III, R^{2a} is (C5-7)cycloalkyl, fused to a 6-membered aromatic or heteroaromatic ring that contains 0, 1 or 2 ring N atoms, wherein R^{2a} is optionally substituted with 1 or more R⁵. R^{2a} may be a (C5-7)cycloalkyl fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with 1 or more fluoro. R^{2a} may be indane optionally substituted with 1 to 3 R⁵, preferably 1 R⁵. In any of the options for embodiment (1), R^{2a} may be optionally substituted with 1 instance of halogen, OH, CN, (C1-4)alkyl or (C1-4)alkoxy.

In embodiment (2) of Formula C or Formula III, R^{2a} is a 5- to 7-membered non-aromatic heterocycle containing one ring O heteroatom, optionally fused to a 6-membered aromatic or heteroaromatic ring that contains 0, 1 or 2 ring N atoms, wherein R^{2a} is optionally substituted with 1 or more R⁵. R^{2a} may be a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro. R^{2a} may be chromane or tetrahydropyran optionally substituted with 1 to 3 R⁵, preferably 1 R⁵. In any of the options for embodiment (2), R^{2a} may be optionally substituted with 1 instance of halogen, OH, CN, (C1-4)alkyl or (C1-4)alkoxy.

According to embodiment (1) or (2) of Formula C or Formula III, R^{2a} may be (C5-6)cycloalkyl fused to a phenyl ring; or a 5- to 6-membered heterocycle containing one ring O heteroatom, optionally fused to a phenyl ring; wherein R^{2a} is optionally substituted.

In embodiment (3) of Formula C or Formula III, R^{2a} is a (C4-6)cycloalkyl group substituted by at least 2 R⁵. R^{2a} may be cyclohexyl, cyclopentyl or cyclobutyl, for example substituted with 2 R⁵. R^{2a} may be a (C5-6)cycloalkyl group substituted by at least 2 R⁵. R^{2a} may be optionally substituted with 2 or more halogen, (C1-4)alkoxy or OH. In any of the options for embodiment (3), R^{2a} may be optionally substituted with 2 or more substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro. R^{2a} may be optionally substituted with 2 or 3 instances of halogen or OH. R^{2a} may be optionally substituted with 2 or 3 instances of halogen, preferably 2 instances of halogen, preferably on the same carbon atom. R^{2a} may be a (C5-6)cycloalkyl group substituted by 2 halogen substituents (optionally on a single ring carbon atom).

In embodiment (4) of Formula C or Formula III,
X is S and Y is N;
R¹ is a 6-membered saturated or aromatic monocyclic ring containing 1 or 2 (optionally 2) ring N heteroatoms or a 7- to 8-membered saturated, bridged ring system containing 1 or 2 (optionally 2) ring N heteroatoms, wherein at least 1 ring N heteroatom is not at the point of attachment of R¹, and wherein R¹ is optionally substituted with 1 R⁴, optionally R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴;
R^{2a} is
(i) (C5-6)cycloalkyl fused to a phenyl ring; or
(ii) a 5- to 6-membered non-aromatic heterocycle containing one ring O heteroatom, optionally fused to a phenyl ring; or
(iv) a (C4-6)cycloalkyl group;
and wherein R^{2a} is optionally substituted with 1 or 2 R⁵, wherein when R^{2a} is (iv) a (C4-6)cycloalkyl group it is substituted by 2 R⁵;
R⁴, where present, is (C1-6)alkyl optionally substituted with OH, optionally (C1-2)alkyl optionally substituted with OH;
R⁵, where present, is OH or halo, optionally OH; and
n is 0 or 1.

According to any of embodiments (1)-(4) of Formula C or Formula III, R^{2a} may be a group of formula

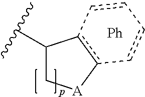

wherein A is O or CH₂; p is 1 or 2; Ph is an optionally present, fused phenyl ring, and wherein R^{2a} is optionally substituted with 1 or 2 R⁵ and wherein when A is CH₂, Ph is present or A is C(R⁵)₂ (i.e CH₂, with two R⁵ substituents, for example, CF₂); optionally wherein when A is O, p is 2 or when A is CH₂, p is 1 or 2. Ph may be absent.

According to any of embodiments (1)-(4) of Formula C or Formula III, R^{2a} may be a group of formula

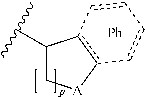

wherein A is O or CH₂; p is 1, 2 or 3; Ph is an optionally present, fused phenyl ring, and wherein R^{2a} is optionally substituted with 1 or 2 R⁵ and wherein when A is CH₂, Ph is present or A is C(R⁵)₂ (for example, CF₂); optionally wherein when A is O, p is 2 or when A is CH₂, p is 1 or 2.

In embodiment (5) of Formula C or Formula III, the compound is a compound of Formula C′ or C″ or Formula IIIa or IIIb:

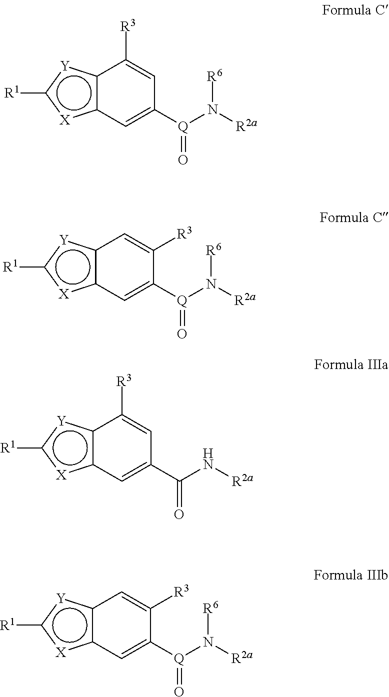

or a pharmaceutically acceptable salt or derivative thereof. R^{2a} may be as defined in relation to any of embodiments (1)-(4) of Formula C or Formula III. In embodiment (6) of Formula C or Formula III, R^{2a} is according to embodiment (2) or embodiment (3) of Formula C or Formula III.

In embodiment (7) of Formula C or Formula III, R¹ is according to embodiment (4) of Formula A or Formula I and R^{2a} is according to embodiment (3) of Formula C or Formula III. The remaining moieties may be as defined for Formula C or Formula III or any of embodiments of Formula C or Formula III described herein, mutatis mutandis. R¹ may be a 7- to 8-membered saturated, bridged ring system containing 1 or 2 ring N heteroatoms, wherein R¹ is optionally substituted with 1 R⁴; and R^{2a} may be according to embodiment (3) of Formula C or Formula III. R¹ may be a 7- to 8-membered saturated, bridged ring system containing 1 or 2 ring N heteroatoms, wherein R¹ is optionally substituted with 1 R⁴; and R^{2a} may be (C4-6)cycloalkyl substituted with 2 or more R⁵. R¹ may be a 7- to 8-membered saturated, bridged ring system containing 1 or 2 ring N heteroatoms, wherein R¹ is optionally substituted with 1 R⁴; and R^{2a} may be (C4-6)cycloalkyl substituted with 2 or more R⁵; optionally wherein R⁵ may be halogen; and n may be 0 or 1.

In embodiment (8) of Formula C or Formula III, when R¹ is 4-morpholinyl, R^{2a} is not 1,2,3,4-tetrahydro-1-naphthalenyl. The remaining moieties may be as defined for Formula C or Formula III or embodiments of Formula C or Formula III described herein, mutatis mutandis. In some embodiments, the compound is not 2-(4-morpholinyl)-N-(1,2,3,4-tetrahydronaphthalenyl)-6-benzothiazolcarboxamide.

In embodiment (9) of Formula C or Formula III, when Q is present and is S(O), R¹ is not optionally substituted pyrazol-4-yl, e.g. optionally substituted

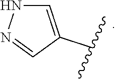 [SMILES: *c1cn[nH]c1]

The remaining moieties may be as defined for Formula C or Formula III or any of embodiments of Formula C or Formula III described herein, mutatis mutandis.

In embodiment (10) of Formula C or Formula III, R¹ is not pyrrol-1-yl. The remaining moieties may be as defined for Formula C or Formula III or embodiments of Formula C or Formula III described herein, mutatis mutandis. In some embodiments, the compound is not N-(2,3-dihydro-1H-inden-2-yl)-2-(1H-pyrrol-1-yl)-6-benzothiazolecarboxamide.

In embodiment (11) of Formula C or Formula III, when R¹ is pyridine or pyrimidine, R^{2a} is not tetrahydro-2-furanyl. When R¹ is pyridine or pyrimidine, R^{2a} may not be tetrahydrofuran. R^{2a} may not be tetrahydrofuran.

Described herein is a compound of Formula D or Formula IV

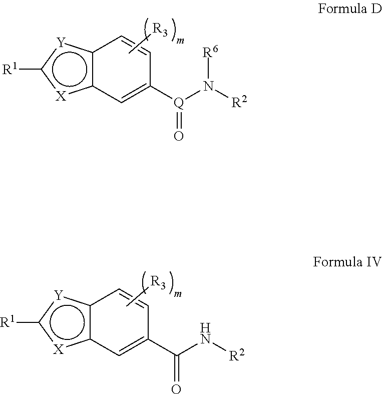

or a pharmaceutically acceptable salt or derivative thereof, wherein m is 1, 2 or 3; and X, Y, Q, R¹, R², R³, R⁴, R⁵ and R⁶ are as defined for Formula A or Formula I or any of embodiments (1)-(17) or (20) of Formula A or Formula I above.

In embodiment (1) of Formula D or Formula IV, m is 1 or 2. m may be 1.

In some embodiments, when Q is S(O), R¹ is not optionally substituted pyrazol-4-yl, e.g. optionally substituted

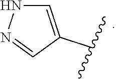 [SMILES: *c1cn[nH]c1]

In embodiment (2) of Formula D or Formula IV, R¹ is not optionally substituted pyrazol-4-yl.

In embodiment (3) of Formula D or Formula IV, the compound is a compound of Formula D′ or D″ or Formula IVa or IVb:

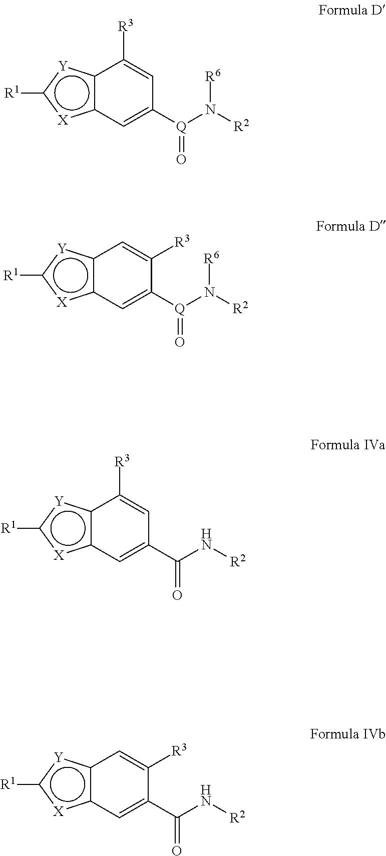

or a pharmaceutically acceptable salt or derivative thereof.

In embodiment (4) of Formula D or Formula IV, the compound is a compound of Formula D′″ or Formula IVc:

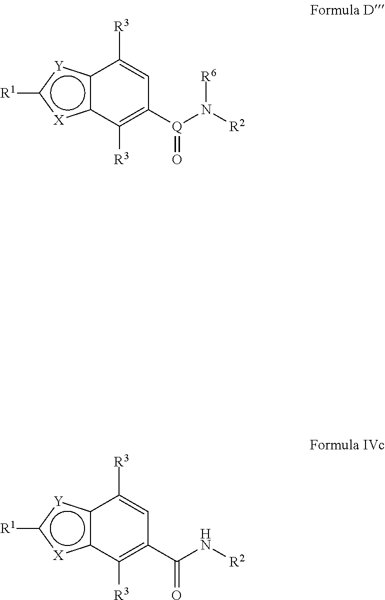

or a pharmaceutically acceptable salt or derivative thereof.

Also disclosed herein are compounds of Formula Z:

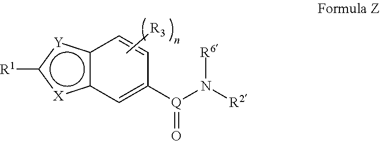

or a pharmaceutically acceptable salt or derivative thereof, wherein R^{2′} and R^{6′} are taken together with the N atom to which they are attached to form a 4- to 7-membered saturated heterocycle, optionally containing 1 further heteroatom selected from O, wherein the 4- to 7-membered saturated heterocycle ring may be optionally substituted with 1 or more R⁵. The remaining moieties X, Y, Q, R¹, R³, R⁴, R⁵ and n may be as defined for any of Formulas A, B, D, I, II or IV or any of embodiments (1)-(8) or (17)-(20) of Formulas A or I or any embodiments of Formulas B, 1l, D or IV described herein, mutatis mutandis. Preferably R^{2′} and R^{6′} are taken together with the N atom to which they are attached to form a 5- to 6-membered saturated heterocycle ring. In compounds of Formula Z, Q is preferably C.

In a further embodiment of a compound of Formula A-D, Formula I-IV or Formula Z or a pharmaceutically acceptable salt thereof, including any of the embodiments thereof described above, one or more hydrogen atoms are replaced by ²H. The remaining moieties may be as defined for any aspect or embodiment of Formula A-D, Formula I-IV or Formula Z described herein, mutatis mutandis.

In an embodiment, the compound of Formula A or Formula I is selected from:
N-(4-chlorobenzyl)-2-(2-methylpyridin-3-yl)benzo[d]thiazole-6-carboxamide;
N-(4-fluorobenzyl)-2-(2-methylpyridin-3-yl)benzo[d]thiazole-6-carboxamide;
N-(4-methoxybenzyl)-2-(2-methylpyridin-3-yl)benzo[d]thiazole-6-carboxamide;
N-(3-fluorobenzyl)-2-(2-methylpyridin-3-yl)benzo[d]thiazole-6-carboxamide;
(S)-N-(2,3-dihydro-1H-inden-1-yl)-2-(2-methylpyridin-3-yl)benzo[d]thiazole-6-carboxamide;
(S)-2-(2-methylpyridin-3-yl)-N-(1,2,3,4-tetrahydronaphthalen-1-yl)benzo[d]thiazole-6-carboxamide;
(S)-N-(chroman-4-yl)-2-(2-methylpyridin-3-yl)benzo[d]thiazole-6-carboxamide;
(S)-2-(pyridin-3-yl)-N-(1,2,3,4-tetrahydronaphthalen-1-yl)benzo[d]thiazole-6-carboxamide;
(S)-N-(chroman-4-yl)-2-(pyridin-3-yl)benzo[d]thiazole-6-carboxamide;
N-benzyl-2-(6-methylpyridin-3-yl)benzo[d]thiazole-6-carboxamide;
N-(4-chlorobenzyl)-2-(6-methylpyridin-3-yl)benzo[d]thiazole-6-carboxamide;
N-(4-fluorobenzyl)-2-(6-methylpyridin-3-yl)benzo[d]thiazole-6-carboxamide;
N-(4-methoxybenzyl)-2-(6-methylpyridin-3-yl)benzo[d]thiazole-6-carboxamide;
N-(3-chlorobenzyl)-2-(6-methylpyridin-3-yl)benzo[d]thiazole-6-carboxamide;
N-(3-fluorobenzyl)-2-(6-methylpyridin-3-yl)benzo[d]thiazole-6-carboxamide;
N-(3-methoxybenzyl)-2-(6-methylpyridin-3-yl)benzo[d]thiazole-6-carboxamide;
(S)-N-(2,3-dihydro-1H-inden-1-yl)-2-(6-methylpyridin-3-yl)benzo[d]thiazole-6-carboxamide;
(R)-N-(2,3-dihydro-1H-inden-1-yl)-2-(6-methylpyridin-3-yl)benzo[d]thiazole-6-carboxamide;
N-((1 S,2R)-2-hydroxy-2,3-dihydro-1H-inden-1-yl)-2-(6-methylpyridin-3-yl)benzo[d]thiazole-6-carboxamide;
N-((1R,2S)-2-hydroxy-2,3-dihydro-1H-inden-1-yl)-2-(6-methylpyridin-3-yl)benzo[d]thiazole-6-carboxamide;
N-((1R,2R)-2-hydroxy-2,3-dihydro-1H-inden-1-yl)-2-(6-methylpyridin-3-yl)benzo[d]thiazole-6-carboxamide;
N-cyclopentyl-2-(6-methylpyridin-3-yl)benzo[d]thiazole-6-carboxamide;
(S)-2-(6-methylpyridin-3-yl)-N-(1,2,3,4-tetrahydronaphthalen-1-yl)benzo[d]thiazole-6-carboxamide;
(S)-N-(chroman-4-yl)-2-(6-methylpyridin-3-yl)benzo[d]thiazole-6-carboxamide;
(S)-N-(chroman-4-yl)-2-(6-(trifluoromethyl)pyridin-3-yl)benzo[d]thiazole-6-carboxamide;
(S)-N-(chroman-4-yl)-2-(2,6-dimethylpyridin-3-yl)benzo[d]thiazole-6-carboxamide;
(S)-N-(chroman-4-yl)-2-(6-cyclopropylpyridin-3-yl)benzo[d]thiazole-6-carboxamide;
(S)-N-(chroman-4-yl)-2-(6-isopropylpyridin-3-yl)benzo[d]thiazole-6-carboxamide;
(S)-N-(chroman-4-yl)-2-(6-ethylpyridin-3-yl)benzo[d]thiazole-6-carboxamide;
N-benzyl-2-(5-methylpyridin-3-yl)benzo[d]thiazole-6-carboxamide;
N-(4-chlorobenzyl)-2-(5-methylpyridin-3-yl)benzo[d]thiazole-6-carboxamide;
N-(4-fluorobenzyl)-2-(5-methylpyridin-3-yl)benzo[d]thiazole-6-carboxamide;
N-(4-methoxybenzyl)-2-(5-methylpyridin-3-yl)benzo[d]thiazole-6-carboxamide;
N-(3-chlorobenzyl)-2-(5-methylpyridin-3-yl)benzo[d]thiazole-6-carboxamide;
N-(3-fluorobenzyl)-2-(5-methylpyridin-3-yl)benzo[d]thiazole-6-carboxamide;
N-(3-methoxybenzyl)-2-(5-methylpyridin-3-yl)benzo[d]thiazole-6-carboxamide;
(S)-N-(2,3-dihydro-1H-inden-1-yl)-2-(5-methylpyridin-3-yl)benzo[d]thiazole-6-carboxamide;
(R)-N-(2,3-dihydro-1H-inden-1-yl)-2-(5-methylpyridin-3-yl)benzo[d]thiazole-6-carboxamide;
N-((1R,2S)-2-hydroxy-2,3-dihydro-1H-inden-1-yl)-2-(5-methylpyridin-3-yl)benzo[d]thiazole-6-carboxamide;
N-((1 S,2R)-2-hydroxy-2,3-dihydro-1H-inden-1-yl)-2-(5-methylpyridin-3-yl)benzo[d]thiazole-6-carboxamide;
N-((1 S,2S)-2-hydroxy-2,3-dihydro-1H-inden-1-yl)-2-(5-methylpyridin-3-yl)benzo[d]thiazole-6-carboxamide;
N-((1R,2R)-2-hydroxy-2,3-dihydro-1H-inden-1-yl)-2-(5-methylpyridin-3-yl)benzo[d]thiazole-6-carboxamide;
N-cyclopentyl-2-(5-methylpyridin-3-yl)benzo[d]thiazole-6-carboxamide;
(S)-2-(5-methylpyridin-3-yl)-N-(1,2,3,4-tetrahydronaphthalen-1-yl)benzo[d]thiazole-6-carboxamide;
(S)-N-(chroman-4-yl)-2-(5-methylpyridin-3-yl)benzo[d]thiazole-6-carboxamide;
2-(5-methylpyridin-3-yl)-N-(tetrahydro-2H-pyran-4-yl)benzo[d]thiazole-6-carboxamide;
(S)-N-(chroman-4-yl)-2-(5-(trifluoromethyl)pyridin-3-yl)benzo[d]thiazole-6-carboxamide;
(S)-N-(chroman-4-yl)-2-(5-cyclopropylpyridin-3-yl)benzo[d]thiazole-6-carboxamide;
(S)-N-(chroman-4-yl)-2-(5-isopropylpyridin-3-yl)benzo[d]thiazole-6-carboxamide;
(S)-2-(4-methylpyridin-3-yl)-N-(1,2,3,4-tetrahydronaphthalen-1-yl)benzo[d]thiazole-6-carboxamide;
(S)-N-(chroman-4-yl)-2-(4-methylpyridin-3-yl)benzo[d]thiazole-6-carboxamide;
(S)-N-(chroman-4-yl)-2-(2,4-dimethylpyridin-3-yl)benzo[d]thiazole-6-carboxamide;
(S)-2-(5-chloropyridin-3-yl)-N-(chroman-4-yl)benzo[d]thiazole-6-carboxamide;
(S)-N-(chroman-4-yl)-2-(pyridin-4-yl)benzo[d]thiazole-6-carboxamide;
(S)-N-(chroman-4-yl)-2-(3-methylpyridin-4-yl)benzo[d]thiazole-6-carboxamide;
(S)-N-(chroman-4-yl)-2-(2-methylpyridin-4-yl)benzo[d]thiazole-6-carboxamide;
(S)-N-(chroman-4-yl)-2-(1-methyl-1H-pyrazol-4-yl)benzo[d]thiazole-6-carboxamide;
(S)-N-(chroman-4-yl)-2-(1,4-dimethyl-1H-pyrazol-5-yl)benzo[d]thiazole-6-carboxamide;
(S)-N-(chroman-4-yl)-2-(1,3,5-trimethyl-1H-pyrazol-4-yl)benzo[d]thiazole-6-carboxamide;
(S)-N-(chroman-4-yl)-2-(1,5-dimethyl-1H-pyrazol-4-yl)benzo[d]thiazole-6-carboxamide;
(S)-N-(chroman-4-yl)-2-(1,3-dimethyl-1H-pyrazol-4-yl)benzo[d]thiazole-6-carboxamide;
(S)-N-(chroman-4-yl)-2-(5-(hydroxymethyl)pyridin-3-yl)benzo[d]thiazole-6-carboxamide;
(S)-N-(chroman-4-yl)-2-(5-(difluoromethyl)pyridin-3-yl)benzo[d]thiazole-6-carboxamide;
(S)-N-(chroman-4-yl)-2-(6-(difluoromethyl)pyridin-3-yl)benzo[d]thiazole-6-carboxamide;
(S)-N-(chroman-4-yl)-2-(2-methylpyridin-3-yl)benzo[d]thiazole-5-carboxamide;
(S)-N-(chroman-4-yl)-2-(4-methylpyridin-3-yl)benzo[d]thiazole-5-carboxamide;
(S)-N-(chroman-4-yl)-2-(pyridin-3-yl)benzo[d]thiazole-5-carboxamide;
(S)-N-(chroman-4-yl)-2-(4-hydroxypiperidin-1-yl)benzo[d]thiazole-6-carboxamide;
(S)-N-(chroman-4-yl)-2-morpholinobenzo[d]thiazole-6-carboxamide;
(S)-N-(chroman-4-yl)-2-(4-methoxypiperidin-1-yl)benzo[d]thiazole-6-carboxamide;
(S)-N-(chroman-4-yl)-2-(4-methylpiperazin-1-yl)benzo[d]thiazole-6-carboxamide;
(S)-N-(chroman-4-yl)-2-(4-ethylpiperazin-1-yl)benzo[d]thiazole-6-carboxamide;
(R)-N-(chroman-4-yl)-2-(4-ethylpiperazin-1-yl)benzo[d]thiazole-6-carboxamide;
(S)-N-(chroman-4-yl)-2-(4-isopropylpiperazin-1-yl)benzo[d]thiazole-6-carboxamide;
(S)-2-(4-(tert-butyl)piperazin-1-yl)-N-(chroman-4-yl)benzo[d]thiazole-6-carboxamide;
(S)-N-(chroman-4-yl)-2-(piperidin-1-yl)benzo[d]thiazole-6-carboxamide;
2-((1S,4S)-2,5-diazabicyclo[2.2.1]heptan-2-yl)-N-((S)-chroman-4-yl)benzo[d]thiazole-6-carboxamide;
2-((1R,4R)-2,5-diazabicyclo[2.2.1]heptan-2-yl)-N-((S)-chroman-4-yl)benzo[d]thiazole-6-carboxamide;
2-(2,5-diazabicyclo[2.2.2]octan-2-yl)-N-((S)-chroman-4-yl)benzo[d]thiazole-6-carboxamide;
2-(4-ethylpiperazin-1-yl)-N-(4-fluorobenzyl)benzo[d]thiazole-6-carboxamide;
N-cyclopentyl-2-(4-ethylpiperazin-1-yl)benzo[d]thiazole-6-carboxamide;
2-(4-ethylpiperazin-1-yl)-N-isopropylbenzo[d]thiazole-6-carboxamide;
(S)-N-(chroman-4-yl)-2-(piperazin-1-yl)benzo[d]thiazole-6-carboxamide;
(R)-N-(chroman-4-yl)-2-(piperazin-1-yl)benzo[d]thiazole-6-carboxamide;
(S)-N-(8-fluorochroman-4-yl)-2-(piperazin-1-yl)benzo[d]thiazole-6-carboxamide;
(R)-N-(8-fluorochroman-4-yl)-2-(piperazin-1-yl)benzo[d]thiazole-6-carboxamide;
(S)-N-(7-fluorochroman-4-yl)-2-(piperazin-1-yl)benzo[d]thiazole-6-carboxamide;
(R)-N-(7-fluorochroman-4-yl)-2-(piperazin-1-yl)benzo[d]thiazole-6-carboxamide;
(S)-N-(6-fluorochroman-4-yl)-2-(piperazin-1-yl)benzo[d]thiazole-6-carboxamide;
(S)-N-(5-fluorochroman-4-yl)-2-(piperazin-1-yl)benzo[d]thiazole-6-carboxamide;
(R)-N-(5-fluorochroman-4-yl)-2-(piperazin-1-yl)benzo[d]thiazole-6-carboxamide;
(S)-N-(7-methoxychroman-4-yl)-2-(piperazin-1-yl)benzo[d]thiazole-6-carboxamide;
(R)-N-(7-methoxychroman-4-yl)-2-(piperazin-1-yl)benzo[d]thiazole-6-carboxamide;
(S)-N-(6-methoxychroman-4-yl)-2-(piperazin-1-yl)benzo[d]thiazole-6-carboxamide;
(S)-N-(7-cyanochroman-4-yl)-2-(piperazin-1-yl)benzo[d]thiazole-6-carboxamide;
(S)-N-(2,3-dihydro-1H-inden-1-yl)-2-(piperazin-1-yl)benzo[d]thiazole-6-carboxamide;
(R)-N-(2,3-dihydro-1H-inden-1-yl)-2-(piperazin-1-yl)benzo[d]thiazole-6-carboxamide;
(S)-N-(7-fluoro-2,3-dihydro-1H-inden-1-yl)-2-(piperazin-1-yl)benzo[d]thiazole-6-carboxamide;
(R)-N-(7-fluoro-2,3-dihydro-1H-inden-1-yl)-2-(piperazin-1-yl)benzo[d]thiazole-6-carboxamide;
(S)-N-(6-fluoro-2,3-dihydro-1H-inden-1-yl)-2-(piperazin-1-yl)benzo[d]thiazole-6-carboxamide;
(S)-N-(5-fluoro-2,3-dihydro-1H-inden-1-yl)-2-(piperazin-1-yl)benzo[d]thiazole-6-carboxamide;
(R)-N-(5-fluoro-2,3-dihydro-1H-inden-1-yl)-2-(piperazin-1-yl)benzo[d]thiazole-6-carboxamide;
(S)-N-(4-fluoro-2,3-dihydro-1H-inden-1-yl)-2-(piperazin-1-yl)benzo[d]thiazole-6-carboxamide;
(R)-N-(4-fluoro-2,3-dihydro-1H-inden-1-yl)-2-(piperazin-1-yl)benzo[d]thiazole-6-carboxamide;
(S)-N-(7-methoxy-2,3-dihydro-1H-inden-1-yl)-2-(piperazin-1-yl)benzo[d]thiazole-6-carboxamide;
(S)-N-(6-methoxy-2,3-dihydro-1H-inden-1-yl)-2-(piperazin-1-yl)benzo[d]thiazole-6-carboxamide;
(R)-N-(6-methoxy-2,3-dihydro-1H-inden-1-yl)-2-(piperazin-1-yl)benzo[d]thiazole-6-carboxamide;
(S)-N-(5-methoxy-2,3-dihydro-1H-inden-1-yl)-2-(piperazin-1-yl)benzo[d]thiazole-6-carboxamide;
(S)-N-(4-methoxy-2,3-dihydro-1H-inden-1-yl)-2-(piperazin-1-yl)benzo[d]thiazole-6-carboxamide;
(R)-N-(4-methoxy-2,3-dihydro-1H-inden-1-yl)-2-(piperazin-1-yl)benzo[d]thiazole-6-carboxamide;
(S)-N-(6-cyano-2,3-dihydro-1H-inden-1-yl)-2-(piperazin-1-yl)benzo[d]thiazole-6-carboxamide;
(R)-N-(6-cyano-2,3-dihydro-1H-inden-1-yl)-2-(piperazin-1-yl)benzo[d]thiazole-6-carboxamide;
(S)-N-(5-cyano-2,3-dihydro-1H-inden-1-yl)-2-(piperazin-1-yl)benzo[d]thiazole-6-carboxamide;
(R)-N-(5-cyano-2,3-dihydro-1H-inden-1-yl)-2-(piperazin-1-yl)benzo[d]thiazole-6-carboxamide;
(S)-N-(4-cyano-2,3-dihydro-1H-inden-1-yl)-2-(piperazin-1-yl)benzo[d]thiazole-6-carboxamide;
(R)-N-(4-cyano-2,3-dihydro-1H-inden-1-yl)-2-(piperazin-1-yl)benzo[d]thiazole-6-carboxamide;
N-cyclopentyl-2-(piperazin-1-yl)benzo[d]thiazole-6-carboxamide;
N-cyclohexyl-2-(piperazin-1-yl)benzo[d]thiazole-6-carboxamide;
N-(4,4-difluorocyclohexyl)-2-(piperazin-1-yl)benzo[d]thiazole-6-carboxamide;
(S)-N-(chroman-4-yl)-2-(4-(2-hydroxyethyl)piperazin-1-yl)benzo[d]thiazole-6-carboxamide;
2-(3,8-diazabicyclo[3.2.1]octan-3-yl)-N-((S)-chroman-4-yl)benzo[d]thiazole-6-carboxamide;
2-(3,8-diazabicyclo[3.2.1]octan-8-yl)-N-((S)-chroman-4-yl)benzo[d]thiazole-6-carboxamide;
N-(4-chlorobenzyl)-2-(piperidin-4-yl)benzo[d]thiazole-6-carboxamide;
N-(4-fluorobenzyl)-2-(piperidin-4-yl)benzo[d]thiazole-6-carboxamide;
N-(4-methoxybenzyl)-2-(piperidin-4-yl)benzo[d]thiazole-6-carboxamide;
N-(4-cyanobenzyl)-2-(piperidin-4-yl)benzo[d]thiazole-6-carboxamide;
(S)-N-(2,3-dihydro-1H-inden-1-yl)-2-(piperidin-4-yl)benzo[d]thiazole-6-carboxamide;
N-((1R,2R)-2-hydroxy-2,3-dihydro-1H-inden-1-yl)-2-(piperidin-4-yl)benzo[d]thiazole-6-carboxamide;
N-((1 S,2S)-2-hydroxy-2,3-dihydro-1H-inden-1-yl)-2-(piperidin-4-yl)benzo[d]thiazole-6-carboxamide;
N-((1R,2S)-2-hydroxy-2,3-dihydro-1H-inden-1-yl)-2-(piperidin-4-yl)benzo[d]thiazole-6-carboxamide;
N-((1 S,2R)-2-hydroxy-2,3-dihydro-1H-inden-1-yl)-2-(piperidin-4-yl)benzo[d]thiazole-6-carboxamide;
(S)-N-(chroman-4-yl)-2-(piperidin-4-yl)benzo[d]thiazole-6-carboxamide;
N-isopropyl-2-(piperidin-4-yl)benzo[d]thiazole-6-carboxamide;
N-cyclopentyl-2-(piperidin-4-yl)benzo[d]thiazole-6-carboxamide;
N-cyclohexyl-2-(piperidin-4-yl)benzo[d]thiazole-6-carboxamide;
N-(4,4-difluorocyclohexyl)-2-(piperidin-4-yl)benzo[d]thiazole-6-carboxamide;
N-(3,3-difluorocyclobutyl)-2-(piperidin-4-yl)benzo[d]thiazole-6-carboxamide;
2-(piperidin-4-yl)-N-(tetrahydro-2H-pyran-4-yl)benzo[d]thiazole-6-carboxamide;
(S)-N-(2-methoxy-1-phenylethyl)-2-(piperidin-4-yl)benzo[d]thiazole-6-carboxamide;
(R)-N-(2-methoxy-1-phenylethyl)-2-(piperidin-4-yl)benzo[d]thiazole-6-carboxamide;
(R)-N-(2-hydroxy-1-phenylethyl)-2-(piperidin-4-yl)benzo[d]thiazole-6-carboxamide;
N-benzyl-2-(1-methylpiperidin-4-yl)benzo[d]thiazole-6-carboxamide;
N-(4-chlorobenzyl)-2-(1-methylpiperidin-4-yl)benzo[d]thiazole-6-carboxamide;
N-(4-fluorobenzyl)-2-(1-methylpiperidin-4-yl)benzo[d]thiazole-6-carboxamide;
N-(4-methoxybenzyl)-2-(1-methylpiperidin-4-yl)benzo[d]thiazole-6-carboxamide;
N-(4-cyanobenzyl)-2-(1-methylpiperidin-4-yl)benzo[d]thiazole-6-carboxamide;
N-(4-methylbenzyl)-2-(1-methylpiperidin-4-yl)benzo[d]thiazole-6-carboxamide;
N-((1R,2S)-2-hydroxy-2,3-dihydro-1H-inden-1-yl)-2-(1-methylpiperidin-4-yl)benzo[d]thiazole-6-carboxamide;
N-((1R,2R)-2-hydroxy-2,3-dihydro-1H-inden-1-yl)-2-(1-methylpiperidin-4-yl)benzo[d]thiazole-6-carboxamide;
(S)-N-(chroman-4-yl)-2-(1-methylpiperidin-4-yl)benzo[d]thiazole-6-carboxamide;
N-cyclopentyl-2-(1-methylpiperidin-4-yl)benzo[d]thiazole-6-carboxamide;
2-(1-methylpiperidin-4-yl)-N-(tetrahydro-2H-pyran-4-yl)benzo[d]thiazole-6-carboxamide;
N-cyclohexyl-2-(1-methylpiperidin-4-yl)benzo[d]thiazole-6-carboxamide;
N-(4,4-difluorocyclohexyl)-2-(1-methylpiperidin-4-yl)benzo[d]thiazole-6-carboxamide;
N-isopropyl-2-(1-methylpiperidin-4-yl)benzo[d]thiazole-6-carboxamide;
N-cyclobutyl-2-(1-methylpiperidin-4-yl)benzo[d]thiazole-6-carboxamide;
N-(3,3-difluorocyclobutyl)-2-(1-methylpiperidin-4-yl)benzo[d]thiazole-6-carboxamide;
(S)-N-(2-methoxy-1-phenylethyl)-2-(1-methylpiperidin-4-yl)benzo[d]thiazole-6-carboxamide;
(R)-N-(2-methoxy-1-phenylethyl)-2-(1-methylpiperidin-4-yl)benzo[d]thiazole-6-carboxamide;
(S)-N-(2-hydroxy-1-phenylethyl)-2-(1-methylpiperidin-4-yl)benzo[d]thiazole-6-carboxamide;
(R)-N-(2-hydroxy-1-phenylethyl)-2-(1-methylpiperidin-4-yl)benzo[d]thiazole-6-carboxamide;
N-cyclopentyl-2-(1-ethylpiperidin-4-yl)benzo[d]thiazole-6-carboxamide;
2-(1-ethylpiperidin-4-yl)-N-(tetrahydro-2H-pyran-4-yl)benzo[d]thiazole-6-carboxamide;
2-(1-ethylpiperidin-4-yl)-N-((1R,2R)-2-hydroxy-2,3-dihydro-1H-inden-1-yl)benzo[d]thiazole-6-carboxamide;
2-(1-ethylpiperidin-4-yl)-N-((1 S,2S)-2-hydroxy-2,3-dihydro-1H-inden-1-yl)benzo[d]thiazole-6-carboxamide;
2-(1-ethylpiperidin-4-yl)-N-((1R,2S)-2-hydroxy-2,3-dihydro-1H-inden-1-yl)benzo[d]thiazole-6-carboxamide;
2-(1-ethylpiperidin-4-yl)-N-((1 S,2R)-2-hydroxy-2,3-dihydro-1H-inden-1-yl)benzo[d]thiazole-6-carboxamide;
(S)-N-(chroman-4-yl)-2-(1-ethylpiperidin-4-yl)benzo[d]thiazole-6-carboxamide;
(S)-N-(2,3-dihydro-1H-inden-1-yl)-2-(1-(2-hydroxyethyl)piperidin-4-yl)benzo[d]thiazole-6-carboxamide;
(S)-N-(chroman-4-yl)-2-(1-(2-hydroxyethyl)piperidin-4-yl)benzo[d]thiazole-6-carboxamide;
N-cyclopentyl-2-(1-(2-hydroxyethyl)piperidin-4-yl)benzo[d]thiazole-6-carboxamide;
2-(1-(2-hydroxyethyl)piperidin-4-yl)-N-(tetrahydro-2H-pyran-4-yl)benzo[d]thiazole-6-carboxamide;
N-cyclohexyl-2-(1-(2-hydroxyethyl)piperidin-4-yl)benzo[d]thiazole-6-carboxamide;
N-(4,4-difluorocyclohexyl)-2-(1-(2-hydroxyethyl)piperidin-4-yl)benzo[d]thiazole-6-carboxamide;
(S)-N-(chroman-4-yl)-2-(1-(2-(2-methoxyethoxy)ethyl)piperidin-4-yl)benzo[d]thiazole-6-carboxamide;
N-((S)-chroman-4-yl)-2-((S)-piperidin-3-yl)benzo[d]thiazole-6-carboxamide;
N-((S)-chroman-4-yl)-2-((R)-piperidin-3-yl)benzo[d]thiazole-6-carboxamide;
N-((S)-chroman-4-yl)-2-(pyrrolidin-3-yl)benzo[d]thiazole-6-carboxamide;
(S)-N-(2,3-dihydro-1H-inden-1-yl)-4-methyl-2-(piperidin-4-yl)benzo[d]thiazole-6-carboxamide;
(S)-N-(2,3-dihydro-1H-inden-1-yl)-2-(1-ethylpiperidin-4-yl)-4-methylbenzo[d]thiazole-6-carboxamide;
N-cyclopentyl-4-methyl-2-(piperidin-4-yl)benzo[d]thiazole-6-carboxamide;
N-cyclopentyl-2-(1-ethylpiperidin-4-yl)-4-methylbenzo[d]thiazole-6-carboxamide;
N-cyclopentyl-4-methyl-2-(1-methylpiperidin-4-yl)benzo[d]thiazole-6-carboxamide;
4-methyl-2-(piperidin-4-yl)-N-(tetrahydro-2H-pyran-4-yl)benzo[d]thiazole-6-carboxamide;
2-(1-ethylpiperidin-4-yl)-4-methyl-N-(tetrahydro-2H-pyran-4-yl)benzo[d]thiazole-6-carboxamide;
(S)-N-(chroman-4-yl)-4-methyl-2-(piperidin-4-yl)benzo[d]thiazole-6-carboxamide;
(S)-N-(chroman-4-yl)-2-(1-ethylpiperidin-4-yl)-4-methylbenzo[d]thiazole-6-carboxamide;
N-cyclopentyl-5-methyl-2-(piperidin-4-yl)benzo[d]thiazole-6-carboxamide;
N-cyclopentyl-2-(1-ethylpiperidin-4-yl)-5-methylbenzo[d]thiazole-6-carboxamide;
(S)-N-(chroman-4-yl)-5-methyl-2-(piperidin-4-yl)benzo[d]thiazole-6-carboxamide;
(S)-N-(chroman-4-yl)-2-(1-ethylpiperidin-4-yl)-5-methylbenzo[d]thiazole-6-carboxamide;
N-cyclopentyl-7-methyl-2-(piperidin-4-yl)benzo[d]thiazole-6-carboxamide;
N-cyclopentyl-2-(1-ethylpiperidin-4-yl)-7-methylbenzo[d]thiazole-6-carboxamide;
(S)-N-(chroman-4-yl)-7-methyl-2-(piperidin-4-yl)benzo[d]thiazole-6-carboxamide;
(S)-N-(chroman-4-yl)-2-(1-ethylpiperidin-4-yl)-7-methylbenzo[d]thiazole-6-carboxamide;
(S)-N-(2,3-dihydro-1H-inden-1-yl)-2-(1-ethylpiperidin-4-yl)-7-methylbenzo[d]thiazole-6-carboxamide;
2-(1-ethylpiperidin-4-yl)-7-methyl-N-(tetrahydro-2H-pyran-4-yl)benzo[d]thiazole-6-carboxamide;
(S)-N-(2,3-dihydro-1H-inden-1-yl)-5-methoxy-2-(piperidin-4-yl)benzo[d]thiazole-6-carboxamide;
N-cyclopentyl-5-methoxy-2-(piperidin-4-yl)benzo[d]thiazole-6-carboxamide;
5-methoxy-2-(piperidin-4-yl)-N-(tetrahydro-2H-pyran-4-yl)benzo[d]thiazole-6-carboxamide;
(S)-N-(chroman-4-yl)-5-methoxy-2-(piperidin-4-yl)benzo[d]thiazole-6-carboxamide;
(S)-N-(2,3-dihydro-1H-inden-1-yl)-4-methyl-2-(piperazin-1-yl)benzo[d]thiazole-6-carboxamide;
(S)-N-(2,3-dihydro-1H-inden-1-yl)-2-(4-ethylpiperazin-1-yl)-4-methylbenzo[d]thiazole-6-carboxamide;
N-cyclobutyl-4-methyl-2-(piperazin-1-yl)benzo[d]thiazole-6-carboxamide;
N-cyclobutyl-4-methyl-2-(4-methylpiperazin-1-yl)benzo[d]thiazole-6-carboxamide;
N-cyclopentyl-4-methyl-2-(piperazin-1-yl)benzo[d]thiazole-6-carboxamide;
N-cyclopentyl-2-(4-ethylpiperazin-1-yl)-4-methylbenzo[d]thiazole-6-carboxamide;
N-cyclopentyl-4-methyl-2-(4-methylpiperazin-1-yl)benzo[d]thiazole-6-carboxamide;
4-methyl-2-(piperazin-1-yl)-N-(tetrahydro-2H-pyran-4-yl)benzo[d]thiazole-6-carboxamide;
2-(4-ethylpiperazin-1-yl)-4-methyl-N-(tetrahydro-2H-pyran-4-yl)benzo[d]thiazole-6-carboxamide;
(S)-N-(chroman-4-yl)-4-methyl-2-(piperazin-1-yl)benzo[d]thiazole-6-carboxamide;
(S)-N-(chroman-4-yl)-2-(4-ethylpiperazin-1-yl)-4-methylbenzo[d]thiazole-6-carboxamide;
N-cyclopentyl-5-methyl-2-(piperazin-1-yl)benzo[d]thiazole-6-carboxamide;
N-cyclopentyl-2-(4-ethylpiperazin-1-yl)-5-methylbenzo[d]thiazole-6-carboxamide;
(S)-N-(chroman-4-yl)-5-methyl-2-(piperazin-1-yl)benzo[d]thiazole-6-carboxamide;
(S)-N-(chroman-4-yl)-2-(4-ethylpiperazin-1-yl)-5-methylbenzo[d]thiazole-6-carboxamide;
N-cyclopentyl-7-methyl-2-(piperazin-1-yl)benzo[d]thiazole-6-carboxamide;
N-cyclopentyl-2-(4-ethylpiperazin-1-yl)-7-methylbenzo[d]thiazole-6-carboxamide;
(S)-N-(chroman-4-yl)-7-methyl-2-(piperazin-1-yl)benzo[d]thiazole-6-carboxamide;
(S)-N-(chroman-4-yl)-2-(4-ethylpiperazin-1-yl)-7-methylbenzo[d]thiazole-6-carboxamide;
N-cyclopentyl-4-methoxy-2-(piperazin-1-yl)benzo[d]thiazole-6-carboxamide;
N-cyclopentyl-2-(4-ethylpiperazin-1-yl)-4-methoxybenzo[d]thiazole-6-carboxamide;
(S)-N-(chroman-4-yl)-4-methoxy-2-(piperazin-1-yl)benzo[d]thiazole-6-carboxamide;
(S)-N-(2,3-dihydro-1H-inden-1-yl)-5-methoxy-2-(piperazin-1-yl)benzo[d]thiazole-6-carboxamide;
N-cyclopentyl-5-methoxy-2-(piperazin-1-yl)benzo[d]thiazole-6-carboxamide;
N-cyclopentyl-2-(4-ethylpiperazin-1-yl)-5-methoxybenzo[d]thiazole-6-carboxamide;
5-methoxy-2-(piperazin-1-yl)-N-(tetrahydro-2H-pyran-4-yl)benzo[d]thiazole-6-carboxamide;
2-(4-ethylpiperazin-1-yl)-5-methoxy-N-(tetrahydro-2H-pyran-4-yl)benzo[d]thiazole-6-carboxamide;
(S)-N-(chroman-4-yl)-5-methoxy-2-(piperazin-1-yl)benzo[d]thiazole-6-carboxamide;
(S)-N-(chroman-4-yl)-4,7-dimethyl-2-(piperidin-4-yl)benzo[d]thiazole-6-carboxamide;
4-chloro-N-cyclopentyl-2-(1-methylpiperidin-4-yl)benzo[d]thiazole-6-carboxamide;
4-cyclopropyl-N-isopropyl-2-(piperidin-4-yl)benzo[d]thiazole-6-carboxamide;
N-cyclobutyl-4-cyclopropyl-2-(1-methylpiperidin-4-yl)benzo[d]thiazole-6-carboxamide;
4-chloro-N-cyclobutyl-2-(piperazin-1-yl)benzo[d]thiazole-6-carboxamide;
4-chloro-N-cyclopentyl-2-(4-(3-hydroxypropyl)piperazin-1-yl)benzo[d]thiazole-6-carboxamide;
2-(3,8-diazabicyclo[3.2.1]octan-8-yl)-N-cyclopentyl-7-methoxybenzo[d]thiazole-6-carboxamide;
2-(3,8-diazabicyclo[3.2.1]octan-8-yl)-4-bromo-N-cyclopentylbenzo[d]thiazole-6-carboxamide;
2-(3,8-diazabicyclo[3.2.1]octan-8-yl)-4-cyano-N-isopropylbenzo[d]thiazole-6-carboxamide;
2-(3,8-diazabicyclo[3.2.1]octan-8-yl)-N-cyclopentylbenzo[d]thiazole-6-carboxamide;
2-(3,8-diazabicyclo[3.2.1]octan-8-yl)-N-(4,4-difluorocyclohexyl)benzo[d]thiazole-6-carboxamide;
2-(3-cyclopropyl-3,8-diazabicyclo[3.2.1]octan-8-yl)-N-(4,4-difluorocyclohexyl)benzo[d]thiazole-6-carboxamide;
N-(4,4-difluorocyclohexyl)-2-(3-(2-hydroxyethyl)-3,8-diazabicyclo[3.2.1]octan-8-yl)benzo[d]thiazole-6-carboxamide;
N-(4,4-difluorocyclohexyl)-2-(3-(3-hydroxypropyl)-3,8-diazabicyclo[3.2.1]octan-8-yl)benzo[d]thiazole-6-carboxamide;
2-(3-oxa-7,9-diazabicyclo[3.3.1]nonan-9-yl)-N-cyclopentylbenzo[d]thiazole-6-carboxamide;
2-(3,8-diazabicyclo[3.2.1]octan-3-yl)-N-(4,4-difluorocyclohexyl)benzo[d]thiazole-6-carboxamide;
N-(4,4-difluorocyclohexyl)-2-(8-methyl-3,8-diazabicyclo[3.2.1]octan-3-yl)benzo[d]thiazole-6-carboxamide;
N-(4,4-difluorocyclohexyl)-2-(8-(2-hydroxyethyl)-3,8-diazabicyclo[3.2.1]octan-3-yl)benzo[d]thiazole-6-carboxamide;
N-(4,4-difluorocyclohexyl)-2-(8-(3-fluoropropyl)-3,8-diazabicyclo[3.2.1]octan-3-yl)benzo[d]thiazole-6-carboxamide;
2-(3,8-diazabicyclo[3.2.1]octan-8-yl)-N-cyclopentyl-4-(1-hydroxypropan-2-yl)benzo[d]thiazole-6-carboxamide;
2-(3,8-diazabicyclo[3.2.1]octan-8-yl)-N-cyclopentyl-4-(2-hydroxyethyl)benzo[d]thiazole-6-carboxamide;
2-(3,6-diazabicyclo[3.1.1]heptan-6-yl)-N-cyclopentylbenzo[d]thiazole-6-carboxamide;
N-cyclopentyl-2-(4,7-diazaspiro[2.5]octan-7-yl)benzo[d]thiazole-6-carboxamide;
N-cyclohexyl-2-((3aR,6aS)-hexahydropyrrolo[3,4-c]pyrrol-2 (1H)-yl)benzo[d]thiazole-6-carboxamide;
N-cyclopentyl-2-(2,6-diazaspiro[3.3]heptan-2-yl)benzo[d]thiazole-6-carboxamide;
N-cyclopentyl-2-(2,6-diazaspiro[3.4]octan-6-yl)benzo[d]thiazole-6-carboxamide;
N-cyclopentyl-2-(2,7-diazaspiro[3.5]nonan-7-yl)benzo[d]thiazole-6-carboxamide;
N-cyclopentyl-2-(octahydro-4H-pyrrolo[3,2-b]pyridin-4-yl)benzo[d]thiazole-6-carboxamide;
N-cyclopentyl-2-(octahydro-5H-pyrrolo[3,2-c]pyridin-5-yl)benzo[d]thiazole-6-carboxamide;
N-cyclopentyl-2-(4-methyl-4,7-diazaspiro[2.5]octan-7-yl)benzo[d]thiazole-6-carboxamide;
N-cyclohexyl-2-((3aR,6aS)-5-methylhexahydropyrrolo[3,4-c]pyrrol-2 (1H)-yl)benzo[d]thiazole-6-carboxamide;
N-cyclopentyl-2-(6-methyl-2,6-diazaspiro[3.3]heptan-2-yl)benzo[d]thiazole-6-carboxamide;
N-cyclopentyl-2-(2-methyl-2,6-diazaspiro[3.4]octan-6-yl)benzo[d]thiazole-6-carboxamide;
N-cyclopentyl-2-(2-methyl-2,7-diazaspiro[3.5]nonan-7-yl)benzo[d]thiazole-6-carboxamide;
N-cyclopentyl-2-(1-methyloctahydro-4H-pyrrolo[3,2-b]pyridin-4-yl)benzo[d]thiazole-6-carboxamide;
N-cyclopentyl-2-(1-methyloctahydro-5H-pyrrolo[3,2-c]pyridin-5-yl)benzo[d]thiazole-6-carboxamide;
(S)-N-(chroman-4-yl)-N-methyl-2-(piperazin-1-yl)benzo[d]thiazole-6-carboxamide;
(S)-N-(chroman-4-yl)-N-methyl-2-(2-methylpyridin-3-yl)benzo[d]thiazole-6-carboxamide;
N-cyclopentyl-N-methyl-2-(piperidin-4-yl)benzo[d]thiazole-6-carboxamide;
2-(piperidin-4-yl)-N-(pyridin-4-ylmethyl)benzo[d]thiazole-6-carboxamide;
2-(piperidin-4-yl)-N-(pyridin-3-ylmethyl)benzo[d]thiazole-6-carboxamide;
N-((1R,2R)-2-hydroxycyclopentyl)-2-(1-methylpiperidin-4-yl)benzo[d]thiazole-6-carboxamide;
2-(1-ethylpiperidin-4-yl)-N-(pyridin-4-ylmethyl)benzo[d]thiazole-6-carboxamide;
2-(1-ethylpiperidin-4-yl)-N-(pyridin-3-ylmethyl)benzo[d]thiazole-6-carboxamide;
2-(1-ethylpiperidin-4-yl)-N-(pyridazin-4-ylmethyl)benzo[d]thiazole-6-carboxamide;
2-(3,8-diazabicyclo[3.2.1]octan-8-yl)-N-(2-isopropoxyethyl)benzo[d]thiazole-6-carboxamide;
2-(3,8-diazabicyclo[3.2.1]octan-8-yl)-N-(2-(cyclopentyloxy)ethyl)benzo[d]thiazole-6-carboxamide;
N-cyclopentyl-2-(1,2-dimethyl-1H-imidazol-5-yl)benzo[d]thiazole-6-carboxamide;
N-cyclopentyl-2-(1-ethylazetidin-3-yl)benzo[d]thiazole-6-carboxamide;
rac-N-cyclopentyl-2-((3S,4R)-3-hydroxy-1-methylpiperidin-4-yl)benzo[d]thiazole-6-carboxamide;
N-cyclopentyl-2-(piperidin-4-yl)benzo[d]thiazole-5-carboxamide;
2-(3,8-diazabicyclo[3.2.1]octan-8-yl)-N-cyclopentylbenzo[d]thiazole-5-carboxamide;
N-cyclopentyl-2-(3-methyl-3,8-diazabicyclo[3.2.1]octan-8-yl)benzo[d]thiazole-5-carboxamide;
N-cyclopentyl-2-(4-ethylpiperazin-1-yl)benzo[d]thiazole-6-sulfonamide;
N-cyclopentyl-2-(piperidin-4-yl)benzo[d]thiazole-6-sulfonamide;
N-cyclopentyl-2-(1-methylpiperidin-4-yl)benzo[d]thiazole-6-sulfonamide;
2-(3,8-diazabicyclo[3.2.1]octan-8-yl)-N-(4,4-difluorocyclohexyl)benzo[d]thiazole-6-sulfonamide;
N-(4,4-difluorocyclohexyl)-2-(3-ethyl-3,8-diazabicyclo[3.2.1]octan-8-yl)benzo[d]thiazole-6-sulfonamide;
N-(4,4-difluorocyclohexyl)-2-(piperidin-4-yl)benzo[d]thiazole-6-sulfonamide;
N-(4,4-difluorocyclohexyl)-2-(1-(2-hydroxyethyl)piperidin-4-yl)benzo[d]thiazole-6-sulfonamide;
and pharmaceutically acceptable salts thereof.
In a further embodiment of a compound of Formula A or Formula I, R¹ may be as defined in any of the compounds of Formula A or Formula I, above. In a further embodiment of a compound of Formula A or Formula I, R² may be as defined in any of the compounds of Formula A or Formula I, above.
In an embodiment, the compound of Formula B or Formula II is selected from:
(S)-N-(chroman-4-yl)-2-(4-methylpiperazin-1-yl)benzo[d]thiazole-6-carboxamide;
(S)-N-(chroman-4-yl)-2-(4-ethylpiperazin-1-yl)benzo[d]thiazole-6-carboxamide;
(R)-N-(chroman-4-yl)-2-(4-ethylpiperazin-1-yl)benzo[d]thiazole-6-carboxamide;
(S)-N-(chroman-4-yl)-2-(4-isopropylpiperazin-1-yl)benzo[d]thiazole-6-carboxamide;
(S)-2-(4-(tert-butyl)piperazin-1-yl)-N-(chroman-4-yl)benzo[d]thiazole-6-carboxamide;
2-((1S,4S)-2,5-diazabicyclo[2.2.1]heptan-2-yl)-N-((S)-chroman-4-yl)benzo[d]thiazole-6-carboxamide;
2-((1R,4R)-2,5-diazabicyclo[2.2.1]heptan-2-yl)-N-((S)-chroman-4-yl)benzo[d]thiazole-6-carboxamide;
2-(2,5-diazabicyclo[2.2.2]octan-2-yl)-N-((S)-chroman-4-yl)benzo[d]thiazole-6-carboxamide;
2-(4-ethylpiperazin-1-yl)-N-(4-fluorobenzyl)benzo[d]thiazole-6-carboxamide;
N-cyclopentyl-2-(4-ethylpiperazin-1-yl)benzo[d]thiazole-6-carboxamide;
2-(4-ethylpiperazin-1-yl)-N-isopropylbenzo[d]thiazole-6-carboxamide;
(S)-N-(chroman-4-yl)-2-(piperazin-1-yl)benzo[d]thiazole-6-carboxamide;
(R)-N-(chroman-4-yl)-2-(piperazin-1-yl)benzo[d]thiazole-6-carboxamide;
(S)-N-(8-fluorochroman-4-yl)-2-(piperazin-1-yl)benzo[d]thiazole-6-carboxamide;
(R)-N-(8-fluorochroman-4-yl)-2-(piperazin-1-yl)benzo[d]thiazole-6-carboxamide;
(S)-N-(7-fluorochroman-4-yl)-2-(piperazin-1-yl)benzo[d]thiazole-6-carboxamide;
(R)-N-(7-fluorochroman-4-yl)-2-(piperazin-1-yl)benzo[d]thiazole-6-carboxamide;
(S)-N-(6-fluorochroman-4-yl)-2-(piperazin-1-yl)benzo[d]thiazole-6-carboxamide;
(S)-N-(5-fluorochroman-4-yl)-2-(piperazin-1-yl)benzo[d]thiazole-6-carboxamide;
(R)-N-(5-fluorochroman-4-yl)-2-(piperazin-1-yl)benzo[d]thiazole-6-carboxamide;
(S)-N-(7-methoxychroman-4-yl)-2-(piperazin-1-yl)benzo[d]thiazole-6-carboxamide;
(R)-N-(7-methoxychroman-4-yl)-2-(piperazin-1-yl)benzo[d]thiazole-6-carboxamide;
(S)-N-(6-methoxychroman-4-yl)-2-(piperazin-1-yl)benzo[d]thiazole-6-carboxamide;
(S)-N-(7-cyanochroman-4-yl)-2-(piperazin-1-yl)benzo[d]thiazole-6-carboxamide;
(S)-N-(2,3-dihydro-1H-inden-1-yl)-2-(piperazin-1-yl)benzo[d]thiazole-6-carboxamide;
(R)-N-(2,3-dihydro-1H-inden-1-yl)-2-(piperazin-1-yl)benzo[d]thiazole-6-carboxamide;
(S)-N-(7-fluoro-2,3-dihydro-1H-inden-1-yl)-2-(piperazin-1-yl)benzo[d]thiazole-6-carboxamide;
(R)-N-(7-fluoro-2,3-dihydro-1H-inden-1-yl)-2-(piperazin-1-yl)benzo[d]thiazole-6-carboxamide;
(S)-N-(6-fluoro-2,3-dihydro-1H-inden-1-yl)-2-(piperazin-1-yl)benzo[d]thiazole-6-carboxamide;
(S)-N-(5-fluoro-2,3-dihydro-1H-inden-1-yl)-2-(piperazin-1-yl)benzo[d]thiazole-6-carboxamide;
(R)-N-(5-fluoro-2,3-dihydro-1H-inden-1-yl)-2-(piperazin-1-yl)benzo[d]thiazole-6-carboxamide;
(S)-N-(4-fluoro-2,3-dihydro-1H-inden-1-yl)-2-(piperazin-1-yl)benzo[d]thiazole-6-carboxamide;
(R)-N-(4-fluoro-2,3-dihydro-1H-inden-1-yl)-2-(piperazin-1-yl)benzo[d]thiazole-6-carboxamide;
(S)-N-(7-methoxy-2,3-dihydro-1H-inden-1-yl)-2-(piperazin-1-yl)benzo[d]thiazole-6-carboxamide;
(S)-N-(6-methoxy-2,3-dihydro-1H-inden-1-yl)-2-(piperazin-1-yl)benzo[d]thiazole-6-carboxamide;
(R)-N-(6-methoxy-2,3-dihydro-1H-inden-1-yl)-2-(piperazin-1-yl)benzo[d]thiazole-6-carboxamide;
(S)-N-(5-methoxy-2,3-dihydro-1H-inden-1-yl)-2-(piperazin-1-yl)benzo[d]thiazole-6-carboxamide;
(S)-N-(4-methoxy-2,3-dihydro-1H-inden-1-yl)-2-(piperazin-1-yl)benzo[d]thiazole-6-carboxamide;
(R)-N-(4-methoxy-2,3-dihydro-1H-inden-1-yl)-2-(piperazin-1-yl)benzo[d]thiazole-6-carboxamide;
(S)-N-(6-cyano-2,3-dihydro-1H-inden-1-yl)-2-(piperazin-1-yl)benzo[d]thiazole-6-carboxamide;
(R)-N-(6-cyano-2,3-dihydro-1H-inden-1-yl)-2-(piperazin-1-yl)benzo[d]thiazole-6-carboxamide;
(S)-N-(5-cyano-2,3-dihydro-1H-inden-1-yl)-2-(piperazin-1-yl)benzo[d]thiazole-6-carboxamide;
(R)-N-(5-cyano-2,3-dihydro-1H-inden-1-yl)-2-(piperazin-1-yl)benzo[d]thiazole-6-carboxamide;
(S)-N-(4-cyano-2,3-dihydro-1H-inden-1-yl)-2-(piperazin-1-yl)benzo[d]thiazole-6-carboxamide;
(R)-N-(4-cyano-2,3-dihydro-1H-inden-1-yl)-2-(piperazin-1-yl)benzo[d]thiazole-6-carboxamide;
N-cyclopentyl-2-(piperazin-1-yl)benzo[d]thiazole-6-carboxamide;
N-cyclohexyl-2-(piperazin-1-yl)benzo[d]thiazole-6-carboxamide;
N-(4,4-difluorocyclohexyl)-2-(piperazin-1-yl)benzo[d]thiazole-6-carboxamide;
(S)-N-(chroman-4-yl)-2-(4-(2-hydroxyethyl)piperazin-1-yl)benzo[d]thiazole-6-carboxamide;
2-(3,8-diazabicyclo[3.2.1]octan-3-yl)-N-((S)-chroman-4-yl)benzo[d]thiazole-6-carboxamide;
2-(3,8-diazabicyclo[3.2.1]octan-8-yl)-N-((S)-chroman-4-yl)benzo[d]thiazole-6-carboxamide;
N-(4-chlorobenzyl)-2-(piperidin-4-yl)benzo[d]thiazole-6-carboxamide;
N-(4-fluorobenzyl)-2-(piperidin-4-yl)benzo[d]thiazole-6-carboxamide;
N-(4-methoxybenzyl)-2-(piperidin-4-yl)benzo[d]thiazole-6-carboxamide;
N-(4-cyanobenzyl)-2-(piperidin-4-yl)benzo[d]thiazole-6-carboxamide;
(S)-N-(2,3-dihydro-1H-inden-1-yl)-2-(piperidin-4-yl)benzo[d]thiazole-6-carboxamide;
N-((1R,2R)-2-hydroxy-2,3-dihydro-1H-inden-1-yl)-2-(piperidin-4-yl)benzo[d]thiazole-6-carboxamide;
N-((1 S,2S)-2-hydroxy-2,3-dihydro-1H-inden-1-yl)-2-(piperidin-4-yl)benzo[d]thiazole-6-carboxamide;
N-((1R,2S)-2-hydroxy-2,3-dihydro-1H-inden-1-yl)-2-(piperidin-4-yl)benzo[d]thiazole-6-carboxamide;
N-((1 S,2R)-2-hydroxy-2,3-dihydro-1H-inden-1-yl)-2-(piperidin-4-yl)benzo[d]thiazole-6-carboxamide;
(S)-N-(chroman-4-yl)-2-(piperidin-4-yl)benzo[d]thiazole-6-carboxamide;
N-isopropyl-2-(piperidin-4-yl)benzo[d]thiazole-6-carboxamide;
N-cyclopentyl-2-(piperidin-4-yl)benzo[d]thiazole-6-carboxamide;
N-cyclohexyl-2-(piperidin-4-yl)benzo[d]thiazole-6-carboxamide;
N-(4,4-difluorocyclohexyl)-2-(piperidin-4-yl)benzo[d]thiazole-6-carboxamide;
N-(3,3-difluorocyclobutyl)-2-(piperidin-4-yl)benzo[d]thiazole-6-carboxamide;
2-(piperidin-4-yl)-N-(tetrahydro-2H-pyran-4-yl)benzo[d]thiazole-6-carboxamide;
(S)-N-(2-methoxy-1-phenylethyl)-2-(piperidin-4-yl)benzo[d]thiazole-6-carboxamide;
(R)-N-(2-methoxy-1-phenylethyl)-2-(piperidin-4-yl)benzo[d]thiazole-6-carboxamide;
(R)-N-(2-hydroxy-1-phenylethyl)-2-(piperidin-4-yl)benzo[d]thiazole-6-carboxamide;
N-benzyl-2-(1-methylpiperidin-4-yl)benzo[d]thiazole-6-carboxamide;
N-(4-chlorobenzyl)-2-(1-methylpiperidin-4-yl)benzo[d]thiazole-6-carboxamide;
N-(4-fluorobenzyl)-2-(1-methylpiperidin-4-yl)benzo[d]thiazole-6-carboxamide;
N-(4-methoxybenzyl)-2-(1-methylpiperidin-4-yl)benzo[d]thiazole-6-carboxamide;
N-(4-cyanobenzyl)-2-(1-methylpiperidin-4-yl)benzo[d]thiazole-6-carboxamide;
N-(4-methylbenzyl)-2-(1-methylpiperidin-4-yl)benzo[d]thiazole-6-carboxamide;
N-((1R,2S)-2-hydroxy-2,3-dihydro-1H-inden-1-yl)-2-(1-methylpiperidin-4-yl)benzo[d]thiazole-6-carboxamide;
N-((1R,2R)-2-hydroxy-2,3-dihydro-1H-inden-1-yl)-2-(1-methylpiperidin-4-yl)benzo[d]thiazole-6-carboxamide;
(S)-N-(chroman-4-yl)-2-(1-methylpiperidin-4-yl)benzo[d]thiazole-6-carboxamide;
N-cyclopentyl-2-(1-methylpiperidin-4-yl)benzo[d]thiazole-6-carboxamide;
2-(1-methylpiperidin-4-yl)-N-(tetrahydro-2H-pyran-4-yl)benzo[d]thiazole-6-carboxamide;
N-cyclohexyl-2-(1-methylpiperidin-4-yl)benzo[d]thiazole-6-carboxamide;
N-(4,4-difluorocyclohexyl)-2-(1-methylpiperidin-4-yl)benzo[d]thiazole-6-carboxamide;
N-isopropyl-2-(1-methylpiperidin-4-yl)benzo[d]thiazole-6-carboxamide;
N-cyclobutyl-2-(1-methylpiperidin-4-yl)benzo[d]thiazole-6-carboxamide;
N-(3,3-difluorocyclobutyl)-2-(1-methylpiperidin-4-yl)benzo[d]thiazole-6-carboxamide;
(S)-N-(2-methoxy-1-phenylethyl)-2-(1-methylpiperidin-4-yl)benzo[d]thiazole-6-carboxamide;
(R)-N-(2-methoxy-1-phenylethyl)-2-(1-methylpiperidin-4-yl)benzo[d]thiazole-6-carboxamide;
(S)-N-(2-hydroxy-1-phenylethyl)-2-(1-methylpiperidin-4-yl)benzo[d]thiazole-6-carboxamide;
(R)-N-(2-hydroxy-1-phenylethyl)-2-(1-methylpiperidin-4-yl)benzo[d]thiazole-6-carboxamide;
N-cyclopentyl-2-(1-ethylpiperidin-4-yl)benzo[d]thiazole-6-carboxamide;
2-(1-ethylpiperidin-4-yl)-N-(tetrahydro-2H-pyran-4-yl)benzo[d]thiazole-6-carboxamide;
2-(1-ethylpiperidin-4-yl)-N-((1R,2R)-2-hydroxy-2,3-dihydro-1H-inden-1-yl)benzo[d]thiazole-6-carboxamide;
2-(1-ethylpiperidin-4-yl)-N-((1 S,2S)-2-hydroxy-2,3-dihydro-1H-inden-1-yl)benzo[d]thiazole-6-carboxamide;
2-(1-ethylpiperidin-4-yl)-N-((1R,2S)-2-hydroxy-2,3-dihydro-1H-inden-1-yl)benzo[d]thiazole-6-carboxamide;
2-(1-ethylpiperidin-4-yl)-N-((1 S,2R)-2-hydroxy-2,3-dihydro-1H-inden-1-yl)benzo[d]thiazole-6-carboxamide;
(S)-N-(chroman-4-yl)-2-(1-ethylpiperidin-4-yl)benzo[d]thiazole-6-carboxamide;
(S)-N-(2,3-dihydro-1H-inden-1-yl)-2-(1-(2-hydroxyethyl)piperidin-4-yl)benzo[d]thiazole-6-carboxamide;
(S)-N-(chroman-4-yl)-2-(1-(2-hydroxyethyl)piperidin-4-yl)benzo[d]thiazole-6-carboxamide;
N-cyclopentyl-2-(1-(2-hydroxyethyl)piperidin-4-yl)benzo[d]thiazole-6-carboxamide;
2-(1-(2-hydroxyethyl)piperidin-4-yl)-N-(tetrahydro-2H-pyran-4-yl)benzo[d]thiazole-6-carboxamide;
N-cyclohexyl-2-(1-(2-hydroxyethyl)piperidin-4-yl)benzo[d]thiazole-6-carboxamide;
N-(4,4-difluorocyclohexyl)-2-(1-(2-hydroxyethyl)piperidin-4-yl)benzo[d]thiazole-6-carboxamide;
(S)-N-(chroman-4-yl)-2-(1-(2-(2-methoxyethoxy)ethyl)piperidin-4-yl)benzo[d]thiazole-6-carboxamide;
N-((S)-chroman-4-yl)-2-((S)-piperidin-3-yl)benzo[d]thiazole-6-carboxamide;
N-((S)-chroman-4-yl)-2-((R)-piperidin-3-yl)benzo[d]thiazole-6-carboxamide;
N-((S)-chroman-4-yl)-2-(pyrrolidin-3-yl)benzo[d]thiazole-6-carboxamide;
(S)-N-(2,3-dihydro-1H-inden-1-yl)-4-methyl-2-(piperidin-4-yl)benzo[d]thiazole-6-carboxamide;
(S)-N-(2,3-dihydro-1H-inden-1-yl)-2-(1-ethylpiperidin-4-yl)-4-methylbenzo[d]thiazole-6-carboxamide;
N-cyclopentyl-4-methyl-2-(piperidin-4-yl)benzo[d]thiazole-6-carboxamide;
N-cyclopentyl-2-(1-ethylpiperidin-4-yl)-4-methylbenzo[d]thiazole-6-carboxamide;
N-cyclopentyl-4-methyl-2-(1-methylpiperidin-4-yl)benzo[d]thiazole-6-carboxamide;
4-methyl-2-(piperidin-4-yl)-N-(tetrahydro-2H-pyran-4-yl)benzo[d]thiazole-6-carboxamide;
2-(1-ethylpiperidin-4-yl)-4-methyl-N-(tetrahydro-2H-pyran-4-yl)benzo[d]thiazole-6-carboxamide;
(S)-N-(chroman-4-yl)-4-methyl-2-(piperidin-4-yl)benzo[d]thiazole-6-carboxamide;
(S)-N-(chroman-4-yl)-2-(1-ethylpiperidin-4-yl)-4-methylbenzo[d]thiazole-6-carboxamide;
N-cyclopentyl-5-methyl-2-(piperidin-4-yl)benzo[d]thiazole-6-carboxamide;
N-cyclopentyl-2-(1-ethylpiperidin-4-yl)-5-methylbenzo[d]thiazole-6-carboxamide;
(S)-N-(chroman-4-yl)-5-methyl-2-(piperidin-4-yl)benzo[d]thiazole-6-carboxamide;
(S)-N-(chroman-4-yl)-2-(1-ethylpiperidin-4-yl)-5-methylbenzo[d]thiazole-6-carboxamide;
N-cyclopentyl-7-methyl-2-(piperidin-4-yl)benzo[d]thiazole-6-carboxamide;
N-cyclopentyl-2-(1-ethylpiperidin-4-yl)-7-methylbenzo[d]thiazole-6-carboxamide;
(S)-N-(chroman-4-yl)-7-methyl-2-(piperidin-4-yl)benzo[d]thiazole-6-carboxamide;
(S)-N-(chroman-4-yl)-2-(1-ethylpiperidin-4-yl)-7-methylbenzo[d]thiazole-6-carboxamide;
(S)-N-(2,3-dihydro-1H-inden-1-yl)-2-(1-ethylpiperidin-4-yl)-7-methylbenzo[d]thiazole-6-carboxamide;
2-(1-ethylpiperidin-4-yl)-7-methyl-N-(tetrahydro-2H-pyran-4-yl)benzo[d]thiazole-6-carboxamide;
(S)-N-(2,3-dihydro-1H-inden-1-yl)-5-methoxy-2-(piperidin-4-yl)benzo[d]thiazole-6-carboxamide;
N-cyclopentyl-5-methoxy-2-(piperidin-4-yl)benzo[d]thiazole-6-carboxamide;
5-methoxy-2-(piperidin-4-yl)-N-(tetrahydro-2H-pyran-4-yl)benzo[d]thiazole-6-carboxamide;
(S)-N-(chroman-4-yl)-5-methoxy-2-(piperidin-4-yl)benzo[d]thiazole-6-carboxamide;
(S)-N-(2,3-dihydro-1H-inden-1-yl)-4-methyl-2-(piperazin-1-yl)benzo[d]thiazole-6-carboxamide;
(S)-N-(2,3-dihydro-1H-inden-1-yl)-2-(4-ethylpiperazin-1-yl)-4-methylbenzo[d]thiazole-6-carboxamide;
N-cyclobutyl-4-methyl-2-(piperazin-1-yl)benzo[d]thiazole-6-carboxamide;
N-cyclobutyl-4-methyl-2-(4-methylpiperazin-1-yl)benzo[d]thiazole-6-carboxamide;
N-cyclopentyl-4-methyl-2-(piperazin-1-yl)benzo[d]thiazole-6-carboxamide;
N-cyclopentyl-2-(4-ethylpiperazin-1-yl)-4-methylbenzo[d]thiazole-6-carboxamide;
N-cyclopentyl-4-methyl-2-(4-methylpiperazin-1-yl)benzo[d]thiazole-6-carboxamide;
4-methyl-2-(piperazin-1-yl)-N-(tetrahydro-2H-pyran-4-yl)benzo[d]thiazole-6-carboxamide;
2-(4-ethylpiperazin-1-yl)-4-methyl-N-(tetrahydro-2H-pyran-4-yl)benzo[d]thiazole-6-carboxamide;
(S)-N-(chroman-4-yl)-4-methyl-2-(piperazin-1-yl)benzo[d]thiazole-6-carboxamide;
(S)-N-(chroman-4-yl)-2-(4-ethylpiperazin-1-yl)-4-methylbenzo[d]thiazole-6-carboxamide;
N-cyclopentyl-5-methyl-2-(piperazin-1-yl)benzo[d]thiazole-6-carboxamide;
N-cyclopentyl-2-(4-ethylpiperazin-1-yl)-5-methylbenzo[d]thiazole-6-carboxamide;
(S)-N-(chroman-4-yl)-5-methyl-2-(piperazin-1-yl)benzo[d]thiazole-6-carboxamide;
(S)-N-(chroman-4-yl)-2-(4-ethylpiperazin-1-yl)-5-methylbenzo[d]thiazole-6-carboxamide;
N-cyclopentyl-7-methyl-2-(piperazin-1-yl)benzo[d]thiazole-6-carboxamide;
N-cyclopentyl-2-(4-ethylpiperazin-1-yl)-7-methylbenzo[d]thiazole-6-carboxamide;
(S)-N-(chroman-4-yl)-7-methyl-2-(piperazin-1-yl)benzo[d]thiazole-6-carboxamide;
(S)-N-(chroman-4-yl)-2-(4-ethylpiperazin-1-yl)-7-methylbenzo[d]thiazole-6-carboxamide;
N-cyclopentyl-4-methoxy-2-(piperazin-1-yl)benzo[d]thiazole-6-carboxamide;
N-cyclopentyl-2-(4-ethylpiperazin-1-yl)-4-methoxybenzo[d]thiazole-6-carboxamide;
(S)-N-(chroman-4-yl)-4-methoxy-2-(piperazin-1-yl)benzo[d]thiazole-6-carboxamide;
(S)-N-(2,3-dihydro-1H-inden-1-yl)-5-methoxy-2-(piperazin-1-yl)benzo[d]thiazole-6-carboxamide;
N-cyclopentyl-5-methoxy-2-(piperazin-1-yl)benzo[d]thiazole-6-carboxamide;
N-cyclopentyl-2-(4-ethylpiperazin-1-yl)-5-methoxybenzo[d]thiazole-6-carboxamide;
5-methoxy-2-(piperazin-1-yl)-N-(tetrahydro-2H-pyran-4-yl)benzo[d]thiazole-6-carboxamide;
2-(4-ethylpiperazin-1-yl)-5-methoxy-N-(tetrahydro-2H-pyran-4-yl)benzo[d]thiazole-6-carboxamide;
(S)-N-(chroman-4-yl)-5-methoxy-2-(piperazin-1-yl)benzo[d]thiazole-6-carboxamide;
(S)-N-(chroman-4-yl)-4,7-dimethyl-2-(piperidin-4-yl)benzo[d]thiazole-6-carboxamide;
4-chloro-N-cyclopentyl-2-(1-methylpiperidin-4-yl)benzo[d]thiazole-6-carboxamide;
4-cyclopropyl-N-isopropyl-2-(piperidin-4-yl)benzo[d]thiazole-6-carboxamide;
N-cyclobutyl-4-cyclopropyl-2-(1-methylpiperidin-4-yl)benzo[d]thiazole-6-carboxamide;
4-chloro-N-cyclobutyl-2-(piperazin-1-yl)benzo[d]thiazole-6-carboxamide;
4-chloro-N-cyclopentyl-2-(4-(3-hydroxypropyl)piperazin-1-yl)benzo[d]thiazole-6-carboxamide;
2-(3,8-diazabicyclo[3.2.1]octan-8-yl)-N-cyclopentyl-7-methoxybenzo[d]thiazole-6-carboxamide;
2-(3,8-diazabicyclo[3.2.1]octan-8-yl)-4-bromo-N-cyclopentylbenzo[d]thiazole-6-carboxamide;
2-(3,8-diazabicyclo[3.2.1]octan-8-yl)-4-cyano-N-isopropylbenzo[d]thiazole-6-carboxamide;
2-(3,8-diazabicyclo[3.2.1]octan-8-yl)-N-cyclopentylbenzo[d]thiazole-6-carboxamide;
2-(3,8-diazabicyclo[3.2.1]octan-8-yl)-N-(4,4-difluorocyclohexyl)benzo[d]thiazole-6-carboxamide;
2-(3-cyclopropyl-3,8-diazabicyclo[3.2.1]octan-8-yl)-N-(4,4-difluorocyclohexyl)benzo[d]thiazole-6-carboxamide;
N-(4,4-difluorocyclohexyl)-2-(3-(2-hydroxyethyl)-3,8-diazabicyclo[3.2.1]octan-8-yl)benzo[d]thiazole-6-carboxamide;
N-(4,4-difluorocyclohexyl)-2-(3-(3-hydroxypropyl)-3,8-diazabicyclo[3.2.1]octan-8-yl)benzo[d]thiazole-6-carboxamide;
2-(3-oxa-7,9-diazabicyclo[3.3.1]nonan-9-yl)-N-cyclopentylbenzo[d]thiazole-6-carboxamide;
2-(3,8-diazabicyclo[3.2.1]octan-3-yl)-N-(4,4-difluorocyclohexyl)benzo[d]thiazole-6-carboxamide;
N-(4,4-difluorocyclohexyl)-2-(8-methyl-3,8-diazabicyclo[3.2.1]octan-3-yl)benzo[d]thiazole-6-carboxamide;
N-(4,4-difluorocyclohexyl)-2-(8-(2-hydroxyethyl)-3,8-diazabicyclo[3.2.1]octan-3-yl)benzo[d]thiazole-6-carboxamide;
N-(4,4-difluorocyclohexyl)-2-(8-(3-fluoropropyl)-3,8-diazabicyclo[3.2.1]octan-3-yl)benzo[d]thiazole-6-carboxamide;
2-(3,8-diazabicyclo[3.2.1]octan-8-yl)-N-cyclopentyl-4-(1-hydroxypropan-2-yl)benzo[d]thiazole-6-carboxamide;
2-(3,8-diazabicyclo[3.2.1]octan-8-yl)-N-cyclopentyl-4-(2-hydroxyethyl)benzo[d]thiazole-6-carboxamide;
2-(3,6-diazabicyclo[3.1.1]heptan-6-yl)-N-cyclopentylbenzo[d]thiazole-6-carboxamide;
N-cyclopentyl-2-(4,7-diazaspiro[2.5]octan-7-yl)benzo[d]thiazole-6-carboxamide;
N-cyclohexyl-2-((3aR,6aS)-hexahydropyrrolo[3,4-c]pyrrol-2 (1H)-yl)benzo[d]thiazole-6-carboxamide;
N-cyclopentyl-2-(2,6-diazaspiro[3.3]heptan-2-yl)benzo[d]thiazole-6-carboxamide;
N-cyclopentyl-2-(2,6-diazaspiro[3.4]octan-6-yl)benzo[d]thiazole-6-carboxamide;
N-cyclopentyl-2-(2,7-diazaspiro[3.5]nonan-7-yl)benzo[d]thiazole-6-carboxamide;
N-cyclopentyl-2-(octahydro-4H-pyrrolo[3,2-b]pyridin-4-yl)benzo[d]thiazole-6-carboxamide;
N-cyclopentyl-2-(octahydro-5H-pyrrolo[3,2-c]pyridin-5-yl)benzo[d]thiazole-6-carboxamide;
N-cyclopentyl-2-(4-methyl-4,7-diazaspiro[2.5]octan-7-yl)benzo[d]thiazole-6-carboxamide;
N-cyclohexyl-2-((3aR,6aS)-5-methylhexahydropyrrolo[3,4-c]pyrrol-2 (1H)-yl)benzo[d]thiazole-6-carboxamide;
N-cyclopentyl-2-(6-methyl-2,6-diazaspiro[3.3]heptan-2-yl)benzo[d]thiazole-6-carboxamide;
N-cyclopentyl-2-(2-methyl-2,6-diazaspiro[3.4]octan-6-yl)benzo[d]thiazole-6-carboxamide;
N-cyclopentyl-2-(2-methyl-2,7-diazaspiro[3.5]nonan-7-yl)benzo[d]thiazole-6-carboxamide;
N-cyclopentyl-2-(1-methyloctahydro-4H-pyrrolo[3,2-b]pyridin-4-yl)benzo[d]thiazole-6-carboxamide;
N-cyclopentyl-2-(1-methyloctahydro-5H-pyrrolo[3,2-c]pyridin-5-yl)benzo[d]thiazole-6-carboxamide;
(S)-N-(chroman-4-yl)-N-methyl-2-(piperazin-1-yl)benzo[d]thiazole-6-carboxamide;
N-cyclopentyl-N-methyl-2-(piperidin-4-yl)benzo[d]thiazole-6-carboxamide;
2-(piperidin-4-yl)-N-(pyridin-4-ylmethyl)benzo[d]thiazole-6-carboxamide;
2-(piperidin-4-yl)-N-(pyridin-3-ylmethyl)benzo[d]thiazole-6-carboxamide;
N-((1R,2R)-2-hydroxycyclopentyl)-2-(1-methylpiperidin-4-yl)benzo[d]thiazole-6-carboxamide;
2-(1-ethylpiperidin-4-yl)-N-(pyridin-4-ylmethyl)benzo[d]thiazole-6-carboxamide;
2-(1-ethylpiperidin-4-yl)-N-(pyridin-3-ylmethyl)benzo[d]thiazole-6-carboxamide;
2-(1-ethylpiperidin-4-yl)-N-(pyridazin-4-ylmethyl)benzo[d]thiazole-6-carboxamide;
2-(3,8-diazabicyclo[3.2.1]octan-8-yl)-N-(2-isopropoxyethyl)benzo[d]thiazole-6-carboxamide;
2-(3,8-diazabicyclo[3.2.1]octan-8-yl)-N-(2-(cyclopentyloxy)ethyl)benzo[d]thiazole-6-carboxamide;
N-cyclopentyl-2-(1-ethylazetidin-3-yl)benzo[d]thiazole-6-carboxamide;
rac-N-cyclopentyl-2-((3S,4R)-3-hydroxy-1-methylpiperidin-4-yl)benzo[d]thiazole-6-carboxamide;
N-cyclopentyl-2-(piperidin-4-yl)benzo[d]thiazole-5-carboxamide;
2-(3,8-diazabicyclo[3.2.1]octan-8-yl)-N-cyclopentylbenzo[d]thiazole-5-carboxamide;
N-cyclopentyl-2-(3-methyl-3,8-diazabicyclo[3.2.1]octan-8-yl)benzo[d]thiazole-5-carboxamide;
N-cyclopentyl-2-(4-ethylpiperazin-1-yl)benzo[d]thiazole-6-sulfonamide;
N-cyclopentyl-2-(piperidin-4-yl)benzo[d]thiazole-6-sulfonamide;
N-cyclopentyl-2-(1-methylpiperidin-4-yl)benzo[d]thiazole-6-sulfonamide;
2-(3,8-diazabicyclo[3.2.1]octan-8-yl)-N-(4,4-difluorocyclohexyl)benzo[d]thiazole-6-sulfonamide;
N-(4,4-difluorocyclohexyl)-2-(3-ethyl-3,8-diazabicyclo[3.2.1]octan-8-yl)benzo[d]thiazole-6-sulfonamide;
N-(4,4-difluorocyclohexyl)-2-(piperidin-4-yl)benzo[d]thiazole-6-sulfonamide;
N-(4,4-difluorocyclohexyl)-2-(1-(2-hydroxyethyl)piperidin-4-yl)benzo[d]thiazole-6-sulfonamide;
and pharmaceutically acceptable salts thereof.
In an embodiment, the compound of Formula C or Formula III is selected from:
(S)-N-(2,3-dihydro-1H-inden-1-yl)-2-(2-methylpyridin-3-yl)benzo[d]thiazole-6-carboxamide;
(S)-2-(2-methylpyridin-3-yl)-N-(1,2,3,4-tetrahydronaphthalen-1-yl)benzo[d]thiazole-6-carboxamide;
(S)-N-(chroman-4-yl)-2-(2-methylpyridin-3-yl)benzo[d]thiazole-6-carboxamide;
(S)-2-(pyridin-3-yl)-N-(1,2,3,4-tetrahydronaphthalen-1-yl)benzo[d]thiazole-6-carboxamide;
(S)-N-(chroman-4-yl)-2-(pyridin-3-yl)benzo[d]thiazole-6-carboxamide;
(S)-N-(2,3-dihydro-1H-inden-1-yl)-2-(6-methylpyridin-3-yl)benzo[d]thiazole-6-carboxamide;
(R)-N-(2,3-dihydro-1H-inden-1-yl)-2-(6-methylpyridin-3-yl)benzo[d]thiazole-6-carboxamide;
N-((1 S,2R)-2-hydroxy-2,3-dihydro-1H-inden-1-yl)-2-(6-methylpyridin-3-yl)benzo[d]thiazole-6-carboxamide;
N-((1R,2S)-2-hydroxy-2,3-dihydro-1H-inden-1-yl)-2-(6-methylpyridin-3-yl)benzo[d]thiazole-6-carboxamide;
N-((1R,2R)-2-hydroxy-2,3-dihydro-1H-inden-1-yl)-2-(6-methylpyridin-3-yl)benzo[d]thiazole-6-carboxamide;
(S)-2-(6-methylpyridin-3-yl)-N-(1,2,3,4-tetrahydronaphthalen-1-yl)benzo[d]thiazole-6-carboxamide;
(S)-N-(chroman-4-yl)-2-(6-methylpyridin-3-yl)benzo[d]thiazole-6-carboxamide;
(S)-N-(chroman-4-yl)-2-(6-(trifluoromethyl)pyridin-3-yl)benzo[d]thiazole-6-carboxamide;
(S)-N-(chroman-4-yl)-2-(2,6-dimethylpyridin-3-yl)benzo[d]thiazole-6-carboxamide;
(S)-N-(chroman-4-yl)-2-(6-cyclopropylpyridin-3-yl)benzo[d]thiazole-6-carboxamide;
(S)-N-(chroman-4-yl)-2-(6-isopropylpyridin-3-yl)benzo[d]thiazole-6-carboxamide;
(S)-N-(chroman-4-yl)-2-(6-ethylpyridin-3-yl)benzo[d]thiazole-6-carboxamide;
(S)-N-(2,3-dihydro-1H-inden-1-yl)-2-(5-methylpyridin-3-yl)benzo[d]thiazole-6-carboxamide;
(R)-N-(2,3-dihydro-1H-inden-1-yl)-2-(5-methylpyridin-3-yl)benzo[d]thiazole-6-carboxamide;
N-((1R,2S)-2-hydroxy-2,3-dihydro-1H-inden-1-yl)-2-(5-methylpyridin-3-yl)benzo[d]thiazole-6-carboxamide;
N-((1 S,2R)-2-hydroxy-2,3-dihydro-1H-inden-1-yl)-2-(5-methylpyridin-3-yl)benzo[d]thiazole-6-carboxamide;
N-((1 S,2S)-2-hydroxy-2,3-dihydro-1H-inden-1-yl)-2-(5-methylpyridin-3-yl)benzo[d]thiazole-6-carboxamide;
N-((1R,2R)-2-hydroxy-2,3-dihydro-1H-inden-1-yl)-2-(5-methylpyridin-3-yl)benzo[d]thiazole-6-carboxamide;
(S)-2-(5-methylpyridin-3-yl)-N-(1,2,3,4-tetrahydronaphthalen-1-yl)benzo[d]thiazole-6-carboxamide;
(S)-N-(chroman-4-yl)-2-(5-methylpyridin-3-yl)benzo[d]thiazole-6-carboxamide;
2-(5-methylpyridin-3-yl)-N-(tetrahydro-2H-pyran-4-yl)benzo[d]thiazole-6-carboxamide;
(S)-N-(chroman-4-yl)-2-(5-(trifluoromethyl)pyridin-3-yl)benzo[d]thiazole-6-carboxamide;
(S)-N-(chroman-4-yl)-2-(5-cyclopropylpyridin-3-yl)benzo[d]thiazole-6-carboxamide;
(S)-N-(chroman-4-yl)-2-(5-isopropylpyridin-3-yl)benzo[d]thiazole-6-carboxamide;
(S)-2-(4-methylpyridin-3-yl)-N-(1,2,3,4-tetrahydronaphthalen-1-yl)benzo[d]thiazole-6-carboxamide;
(S)-N-(chroman-4-yl)-2-(4-methylpyridin-3-yl)benzo[d]thiazole-6-carboxamide;
(S)-N-(chroman-4-yl)-2-(2,4-dimethylpyridin-3-yl)benzo[d]thiazole-6-carboxamide;
(S)-2-(5-chloropyridin-3-yl)-N-(chroman-4-yl)benzo[d]thiazole-6-carboxamide;
(S)-N-(chroman-4-yl)-2-(pyridin-4-yl)benzo[d]thiazole-6-carboxamide;
(S)-N-(chroman-4-yl)-2-(3-methylpyridin-4-yl)benzo[d]thiazole-6-carboxamide;
(S)-N-(chroman-4-yl)-2-(2-methylpyridin-4-yl)benzo[d]thiazole-6-carboxamide;
(S)-N-(chroman-4-yl)-2-(1-methyl-1H-pyrazol-4-yl)benzo[d]thiazole-6-carboxamide;
(S)-N-(chroman-4-yl)-2-(1,4-dimethyl-1H-pyrazol-5-yl)benzo[d]thiazole-6-carboxamide;
(S)-N-(chroman-4-yl)-2-(1,3,5-trimethyl-1H-pyrazol-4-yl)benzo[d]thiazole-6-carboxamide;
(S)-N-(chroman-4-yl)-2-(1,5-dimethyl-1H-pyrazol-4-yl)benzo[d]thiazole-6-carboxamide;
(S)-N-(chroman-4-yl)-2-(1,3-dimethyl-1H-pyrazol-4-yl)benzo[d]thiazole-6-carboxamide;
(S)-N-(chroman-4-yl)-2-(5-(hydroxymethyl)pyridin-3-yl)benzo[d]thiazole-6-carboxamide;
(S)-N-(chroman-4-yl)-2-(5-(difluoromethyl)pyridin-3-yl)benzo[d]thiazole-6-carboxamide;
(S)-N-(chroman-4-yl)-2-(6-(difluoromethyl)pyridin-3-yl)benzo[d]thiazole-6-carboxamide;
(S)-N-(chroman-4-yl)-2-(2-methylpyridin-3-yl)benzo[d]thiazole-5-carboxamide;
(S)-N-(chroman-4-yl)-2-(4-methylpyridin-3-yl)benzo[d]thiazole-5-carboxamide;
(S)-N-(chroman-4-yl)-2-(pyridin-3-yl)benzo[d]thiazole-5-carboxamide;
(S)-N-(chroman-4-yl)-2-(4-hydroxypiperidin-1-yl)benzo[d]thiazole-6-carboxamide;
(S)-N-(chroman-4-yl)-2-morpholinobenzo[d]thiazole-6-carboxamide;
(S)-N-(chroman-4-yl)-2-(4-methoxypiperidin-1-yl)benzo[d]thiazole-6-carboxamide;
(S)-N-(chroman-4-yl)-2-(4-methylpiperazin-1-yl)benzo[d]thiazole-6-carboxamide;
(S)-N-(chroman-4-yl)-2-(4-ethylpiperazin-1-yl)benzo[d]thiazole-6-carboxamide;
(R)-N-(chroman-4-yl)-2-(4-ethylpiperazin-1-yl)benzo[d]thiazole-6-carboxamide;
(S)-N-(chroman-4-yl)-2-(4-isopropylpiperazin-1-yl)benzo[d]thiazole-6-carboxamide;
(S)-2-(4-(tert-butyl)piperazin-1-yl)-N-(chroman-4-yl)benzo[d]thiazole-6-carboxamide;
(S)-N-(chroman-4-yl)-2-(piperidin-1-yl)benzo[d]thiazole-6-carboxamide;
2-((1S,4S)-2,5-diazabicyclo[2.2.1]heptan-2-yl)-N-((S)-chroman-4-yl)benzo[d]thiazole-6-carboxamide;
2-((1R,4R)-2,5-diazabicyclo[2.2.1]heptan-2-yl)-N-((S)-chroman-4-yl)benzo[d]thiazole-6-carboxamide;
2-(2,5-diazabicyclo[2.2.2]octan-2-yl)-N-((S)-chroman-4-yl)benzo[d]thiazole-6-carboxamide;
(S)-N-(chroman-4-yl)-2-(piperazin-1-yl)benzo[d]thiazole-6-carboxamide;
(R)-N-(chroman-4-yl)-2-(piperazin-1-yl)benzo[d]thiazole-6-carboxamide;
(S)-N-(8-fluorochroman-4-yl)-2-(piperazin-1-yl)benzo[d]thiazole-6-carboxamide;
(R)-N-(8-fluorochroman-4-yl)-2-(piperazin-1-yl)benzo[d]thiazole-6-carboxamide;
(S)-N-(7-fluorochroman-4-yl)-2-(piperazin-1-yl)benzo[d]thiazole-6-carboxamide;
(R)-N-(7-fluorochroman-4-yl)-2-(piperazin-1-yl)benzo[d]thiazole-6-carboxamide;
(S)-N-(6-fluorochroman-4-yl)-2-(piperazin-1-yl)benzo[d]thiazole-6-carboxamide;
(S)-N-(5-fluorochroman-4-yl)-2-(piperazin-1-yl)benzo[d]thiazole-6-carboxamide;
(R)-N-(5-fluorochroman-4-yl)-2-(piperazin-1-yl)benzo[d]thiazole-6-carboxamide;
(S)-N-(7-methoxychroman-4-yl)-2-(piperazin-1-yl)benzo[d]thiazole-6-carboxamide;
(R)-N-(7-methoxychroman-4-yl)-2-(piperazin-1-yl)benzo[d]thiazole-6-carboxamide;
(S)-N-(6-methoxychroman-4-yl)-2-(piperazin-1-yl)benzo[d]thiazole-6-carboxamide;
(S)-N-(7-cyanochroman-4-yl)-2-(piperazin-1-yl)benzo[d]thiazole-6-carboxamide;
(S)-N-(2,3-dihydro-1H-inden-1-yl)-2-(piperazin-1-yl)benzo[d]thiazole-6-carboxamide;
(R)-N-(2,3-dihydro-1H-inden-1-yl)-2-(piperazin-1-yl)benzo[d]thiazole-6-carboxamide;
(S)-N-(7-fluoro-2,3-dihydro-1H-inden-1-yl)-2-(piperazin-1-yl)benzo[d]thiazole-6-carboxamide;
(R)-N-(7-fluoro-2,3-dihydro-1H-inden-1-yl)-2-(piperazin-1-yl)benzo[d]thiazole-6-carboxamide;
(S)-N-(6-fluoro-2,3-dihydro-1H-inden-1-yl)-2-(piperazin-1-yl)benzo[d]thiazole-6-carboxamide;
(S)-N-(5-fluoro-2,3-dihydro-1H-inden-1-yl)-2-(piperazin-1-yl)benzo[d]thiazole-6-carboxamide;
(R)-N-(5-fluoro-2,3-dihydro-1H-inden-1-yl)-2-(piperazin-1-yl)benzo[d]thiazole-6-carboxamide;
(S)-N-(4-fluoro-2,3-dihydro-1H-inden-1-yl)-2-(piperazin-1-yl)benzo[d]thiazole-6-carboxamide;
(R)-N-(4-fluoro-2,3-dihydro-1H-inden-1-yl)-2-(piperazin-1-yl)benzo[d]thiazole-6-carboxamide;
(S)-N-(7-methoxy-2,3-dihydro-1H-inden-1-yl)-2-(piperazin-1-yl)benzo[d]thiazole-6-carboxamide;
(S)-N-(6-methoxy-2,3-dihydro-1H-inden-1-yl)-2-(piperazin-1-yl)benzo[d]thiazole-6-carboxamide;
(R)-N-(6-methoxy-2,3-dihydro-1H-inden-1-yl)-2-(piperazin-1-yl)benzo[d]thiazole-6-carboxamide;
(S)-N-(5-methoxy-2,3-dihydro-1H-inden-1-yl)-2-(piperazin-1-yl)benzo[d]thiazole-6-carboxamide;
(S)-N-(4-methoxy-2,3-dihydro-1H-inden-1-yl)-2-(piperazin-1-yl)benzo[d]thiazole-6-carboxamide;
(R)-N-(4-methoxy-2,3-dihydro-1H-inden-1-yl)-2-(piperazin-1-yl)benzo[d]thiazole-6-carboxamide;
(S)-N-(6-cyano-2,3-dihydro-1H-inden-1-yl)-2-(piperazin-1-yl)benzo[d]thiazole-6-carboxamide;
(R)-N-(6-cyano-2,3-dihydro-1H-inden-1-yl)-2-(piperazin-1-yl)benzo[d]thiazole-6-carboxamide;
(S)-N-(5-cyano-2,3-dihydro-1H-inden-1-yl)-2-(piperazin-1-yl)benzo[d]thiazole-6-carboxamide;
(R)-N-(5-cyano-2,3-dihydro-1H-inden-1-yl)-2-(piperazin-1-yl)benzo[d]thiazole-6-carboxamide;
(S)-N-(4-cyano-2,3-dihydro-1H-inden-1-yl)-2-(piperazin-1-yl)benzo[d]thiazole-6-carboxamide;
(R)-N-(4-cyano-2,3-dihydro-1H-inden-1-yl)-2-(piperazin-1-yl)benzo[d]thiazole-6-carboxamide;
N-(4,4-difluorocyclohexyl)-2-(piperazin-1-yl)benzo[d]thiazole-6-carboxamide;
(S)-N-(chroman-4-yl)-2-(4-(2-hydroxyethyl)piperazin-1-yl)benzo[d]thiazole-6-carboxamide;
2-(3,8-diazabicyclo[3.2.1]octan-3-yl)-N-((S)-chroman-4-yl)benzo[d]thiazole-6-carboxamide;
2-(3,8-diazabicyclo[3.2.1]octan-8-yl)-N-((S)-chroman-4-yl)benzo[d]thiazole-6-carboxamide;
(S)-N-(2,3-dihydro-1H-inden-1-yl)-2-(piperidin-4-yl)benzo[d]thiazole-6-carboxamide;
N-((1R,2R)-2-hydroxy-2,3-dihydro-1H-inden-1-yl)-2-(piperidin-4-yl)benzo[d]thiazole-6-carboxamide;
N-((1 S,2S)-2-hydroxy-2,3-dihydro-1H-inden-1-yl)-2-(piperidin-4-yl)benzo[d]thiazole-6-carboxamide;
N-((1R,2S)-2-hydroxy-2,3-dihydro-1H-inden-1-yl)-2-(piperidin-4-yl)benzo[d]thiazole-6-carboxamide;
N-((1 S,2R)-2-hydroxy-2,3-dihydro-1H-inden-1-yl)-2-(piperidin-4-yl)benzo[d]thiazole-6-carboxamide;
(S)-N-(chroman-4-yl)-2-(piperidin-4-yl)benzo[d]thiazole-6-carboxamide;
N-(4,4-difluorocyclohexyl)-2-(piperidin-4-yl)benzo[d]thiazole-6-carboxamide;
N-(3,3-difluorocyclobutyl)-2-(piperidin-4-yl)benzo[d]thiazole-6-carboxamide;
2-(piperidin-4-yl)-N-(tetrahydro-2H-pyran-4-yl)benzo[d]thiazole-6-carboxamide;
N-((1R,2S)-2-hydroxy-2,3-dihydro-1H-inden-1-yl)-2-(1-methylpiperidin-4-yl)benzo[d]thiazole-6-carboxamide;
N-((1R,2R)-2-hydroxy-2,3-dihydro-1H-inden-1-yl)-2-(1-methylpiperidin-4-yl)benzo[d]thiazole-6-carboxamide;
(S)-N-(chroman-4-yl)-2-(1-methylpiperidin-4-yl)benzo[d]thiazole-6-carboxamide;
2-(1-methylpiperidin-4-yl)-N-(tetrahydro-2H-pyran-4-yl)benzo[d]thiazole-6-carboxamide;
N-(4,4-difluorocyclohexyl)-2-(1-methylpiperidin-4-yl)benzo[d]thiazole-6-carboxamide;
N-(3,3-difluorocyclobutyl)-2-(1-methylpiperidin-4-yl)benzo[d]thiazole-6-carboxamide;
2-(1-ethylpiperidin-4-yl)-N-(tetrahydro-2H-pyran-4-yl)benzo[d]thiazole-6-carboxamide;
2-(1-ethylpiperidin-4-yl)-N-((1R,2R)-2-hydroxy-2,3-dihydro-1H-inden-1-yl)benzo[d]thiazole-6-carboxamide;
2-(1-ethylpiperidin-4-yl)-N-((1 S,2S)-2-hydroxy-2,3-dihydro-1H-inden-1-yl)benzo[d]thiazole-6-carboxamide;
2-(1-ethylpiperidin-4-yl)-N-((1R,2S)-2-hydroxy-2,3-dihydro-1H-inden-1-yl)benzo[d]thiazole-6-carboxamide;
2-(1-ethylpiperidin-4-yl)-N-((1 S,2R)-2-hydroxy-2,3-dihydro-1H-inden-1-yl)benzo[d]thiazole-6-carboxamide;
(S)-N-(chroman-4-yl)-2-(1-ethylpiperidin-4-yl)benzo[d]thiazole-6-carboxamide;
(S)-N-(2,3-dihydro-1H-inden-1-yl)-2-(1-(2-hydroxyethyl)piperidin-4-yl)benzo[d]thiazole-6-carboxamide;
(S)-N-(chroman-4-yl)-2-(1-(2-hydroxyethyl)piperidin-4-yl)benzo[d]thiazole-6-carboxamide;
2-(1-(2-hydroxyethyl)piperidin-4-yl)-N-(tetrahydro-2H-pyran-4-yl)benzo[d]thiazole-6-carboxamide;
N-(4,4-difluorocyclohexyl)-2-(1-(2-hydroxyethyl)piperidin-4-yl)benzo[d]thiazole-6-carboxamide;
(S)-N-(chroman-4-yl)-2-(1-(2-(2-methoxyethoxy)ethyl)piperidin-4-yl)benzo[d]thiazole-6-carboxamide;
N-((S)-chroman-4-yl)-2-((S)-piperidin-3-yl)benzo[d]thiazole-6-carboxamide;
N-((S)-chroman-4-yl)-2-((R)-piperidin-3-yl)benzo[d]thiazole-6-carboxamide;
N-((S)-chroman-4-yl)-2-(pyrrolidin-3-yl)benzo[d]thiazole-6-carboxamide;
(S)-N-(2,3-dihydro-1H-inden-1-yl)-4-methyl-2-(piperidin-4-yl)benzo[d]thiazole-6-carboxamide;
(S)-N-(2,3-dihydro-1H-inden-1-yl)-2-(1-ethylpiperidin-4-yl)-4-methylbenzo[d]thiazole-6-carboxamide;
4-methyl-2-(piperidin-4-yl)-N-(tetrahydro-2H-pyran-4-yl)benzo[d]thiazole-6-carboxamide;
2-(1-ethylpiperidin-4-yl)-4-methyl-N-(tetrahydro-2H-pyran-4-yl)benzo[d]thiazole-6-carboxamide;
(S)-N-(chroman-4-yl)-4-methyl-2-(piperidin-4-yl)benzo[d]thiazole-6-carboxamide;
(S)-N-(chroman-4-yl)-2-(1-ethylpiperidin-4-yl)-4-methylbenzo[d]thiazole-6-carboxamide;
(S)-N-(chroman-4-yl)-5-methyl-2-(piperidin-4-yl)benzo[d]thiazole-6-carboxamide;
(S)-N-(chroman-4-yl)-2-(1-ethylpiperidin-4-yl)-5-methylbenzo[d]thiazole-6-carboxamide;
(S)-N-(chroman-4-yl)-7-methyl-2-(piperidin-4-yl)benzo[d]thiazole-6-carboxamide;
(S)-N-(chroman-4-yl)-2-(1-ethylpiperidin-4-yl)-7-methylbenzo[d]thiazole-6-carboxamide;
(S)-N-(2,3-dihydro-1H-inden-1-yl)-2-(1-ethylpiperidin-4-yl)-7-methylbenzo[d]thiazole-6-carboxamide;
2-(1-ethylpiperidin-4-yl)-7-methyl-N-(tetrahydro-2H-pyran-4-yl)benzo[d]thiazole-6-carboxamide;
(S)-N-(2,3-dihydro-1H-inden-1-yl)-5-methoxy-2-(piperidin-4-yl)benzo[d]thiazole-6-carboxamide;
5-methoxy-2-(piperidin-4-yl)-N-(tetrahydro-2H-pyran-4-yl)benzo[d]thiazole-6-carboxamide;
(S)-N-(chroman-4-yl)-5-methoxy-2-(piperidin-4-yl)benzo[d]thiazole-6-carboxamide;
(S)-N-(2,3-dihydro-1H-inden-1-yl)-4-methyl-2-(piperazin-1-yl)benzo[d]thiazole-6-carboxamide;
(S)-N-(2,3-dihydro-1H-inden-1-yl)-2-(4-ethylpiperazin-1-yl)-4-methylbenzo[d]thiazole-6-carboxamide;
4-methyl-2-(piperazin-1-yl)-N-(tetrahydro-2H-pyran-4-yl)benzo[d]thiazole-6-carboxamide;
2-(4-ethylpiperazin-1-yl)-4-methyl-N-(tetrahydro-2H-pyran-4-yl)benzo[d]thiazole-6-carboxamide;
(S)-N-(chroman-4-yl)-4-methyl-2-(piperazin-1-yl)benzo[d]thiazole-6-carboxamide;
(S)-N-(chroman-4-yl)-2-(4-ethylpiperazin-1-yl)-4-methylbenzo[d]thiazole-6-carboxamide;
(S)-N-(chroman-4-yl)-5-methyl-2-(piperazin-1-yl)benzo[d]thiazole-6-carboxamide;
(S)-N-(chroman-4-yl)-2-(4-ethylpiperazin-1-yl)-5-methylbenzo[d]thiazole-6-carboxamide;
(S)-N-(chroman-4-yl)-7-methyl-2-(piperazin-1-yl)benzo[d]thiazole-6-carboxamide;
(S)-N-(chroman-4-yl)-2-(4-ethylpiperazin-1-yl)-7-methylbenzo[d]thiazole-6-carboxamide;
(S)-N-(chroman-4-yl)-4-methoxy-2-(piperazin-1-yl)benzo[d]thiazole-6-carboxamide;
(S)-N-(2,3-dihydro-1H-inden-1-yl)-5-methoxy-2-(piperazin-1-yl)benzo[d]thiazole-6-carboxamide;
5-methoxy-2-(piperazin-1-yl)-N-(tetrahydro-2H-pyran-4-yl)benzo[d]thiazole-6-carboxamide;
2-(4-ethylpiperazin-1-yl)-5-methoxy-N-(tetrahydro-2H-pyran-4-yl)benzo[d]thiazole-6-carboxamide;
(S)-N-(chroman-4-yl)-5-methoxy-2-(piperazin-1-yl)benzo[d]thiazole-6-carboxamide;
(S)-N-(chroman-4-yl)-4,7-dimethyl-2-(piperidin-4-yl)benzo[d]thiazole-6-carboxamide;
2-(3,8-diazabicyclo[3.2.1]octan-8-yl)-N-(4,4-difluorocyclohexyl)benzo[d]thiazole-6-carboxamide;
2-(3-cyclopropyl-3,8-diazabicyclo[3.2.1]octan-8-yl)-N-(4,4-difluorocyclohexyl)benzo[d]thiazole-6-carboxamide;
N-(4,4-difluorocyclohexyl)-2-(3-(2-hydroxyethyl)-3,8-diazabicyclo[3.2.1]octan-8-yl)benzo[d]thiazole-6-carboxamide;
N-(4,4-difluorocyclohexyl)-2-(3-(3-hydroxypropyl)-3,8-diazabicyclo[3.2.1]octan-8-yl)benzo[d]thiazole-6-carboxamide;
2-(3,8-diazabicyclo[3.2.1]octan-3-yl)-N-(4,4-difluorocyclohexyl)benzo[d]thiazole-6-carboxamide;
N-(4,4-difluorocyclohexyl)-2-(8-methyl-3,8-diazabicyclo[3.2.1]octan-3-yl)benzo[d]thiazole-6-carboxamide;
N-(4,4-difluorocyclohexyl)-2-(8-(2-hydroxyethyl)-3,8-diazabicyclo[3.2.1]octan-3-yl)benzo[d]thiazole-6-carboxamide;
N-(4,4-difluorocyclohexyl)-2-(8-(3-fluoropropyl)-3,8-diazabicyclo[3.2.1]octan-3-yl)benzo[d]thiazole-6-carboxamide;
(S)-N-(chroman-4-yl)-N-methyl-2-(piperazin-1-yl)benzo[d]thiazole-6-carboxamide;
(S)-N-(chroman-4-yl)-N-methyl-2-(2-methylpyridin-3-yl)benzo[d]thiazole-6-carboxamide;
2-(3,8-diazabicyclo[3.2.1]octan-8-yl)-N-(4,4-difluorocyclohexyl)benzo[d]thiazole-6-sulfonamide;
N-(4,4-difluorocyclohexyl)-2-(3-ethyl-3,8-diazabicyclo[3.2.1]octan-8-yl)benzo[d]thiazole-6-sulfonamide;
N-(4,4-difluorocyclohexyl)-2-(piperidin-4-yl)benzo[d]thiazole-6-sulfonamide;
N-(4,4-difluorocyclohexyl)-2-(1-(2-hydroxyethyl)piperidin-4-yl)benzo[d]thiazole-6-sulfonamide;
and pharmaceutically acceptable salts thereof.

In an embodiment, the compound of Formula D or Formula IV is selected from:
(S)-N-(2,3-dihydro-1H-inden-1-yl)-4-methyl-2-(piperidin-4-yl)benzo[d]thiazole-6-carboxamide;
(S)-N-(2,3-dihydro-1H-inden-1-yl)-2-(1-ethylpiperidin-4-yl)-4-methylbenzo[d]thiazole-6-carboxamide;
N-cyclopentyl-4-methyl-2-(piperidin-4-yl)benzo[d]thiazole-6-carboxamide;
N-cyclopentyl-2-(1-ethylpiperidin-4-yl)-4-methylbenzo[d]thiazole-6-carboxamide;
N-cyclopentyl-4-methyl-2-(1-methylpiperidin-4-yl)benzo[d]thiazole-6-carboxamide;
4-methyl-2-(piperidin-4-yl)-N-(tetrahydro-2H-pyran-4-yl)benzo[d]thiazole-6-carboxamide;
2-(1-ethylpiperidin-4-yl)-4-methyl-N-(tetrahydro-2H-pyran-4-yl)benzo[d]thiazole-6-carboxamide;
(S)-N-(chroman-4-yl)-4-methyl-2-(piperidin-4-yl)benzo[d]thiazole-6-carboxamide;
(S)-N-(chroman-4-yl)-2-(1-ethylpiperidin-4-yl)-4-methylbenzo[d]thiazole-6-carboxamide;
N-cyclopentyl-5-methyl-2-(piperidin-4-yl)benzo[d]thiazole-6-carboxamide;
N-cyclopentyl-2-(1-ethylpiperidin-4-yl)-5-methylbenzo[d]thiazole-6-carboxamide;
(S)-N-(chroman-4-yl)-5-methyl-2-(piperidin-4-yl)benzo[d]thiazole-6-carboxamide;
(S)-N-(chroman-4-yl)-2-(1-ethylpiperidin-4-yl)-5-methylbenzo[d]thiazole-6-carboxamide;
N-cyclopentyl-7-methyl-2-(piperidin-4-yl)benzo[d]thiazole-6-carboxamide;
N-cyclopentyl-2-(1-ethylpiperidin-4-yl)-7-methylbenzo[d]thiazole-6-carboxamide;
(S)-N-(chroman-4-yl)-7-methyl-2-(piperidin-4-yl)benzo[d]thiazole-6-carboxamide;
(S)-N-(chroman-4-yl)-2-(1-ethylpiperidin-4-yl)-7-methylbenzo[d]thiazole-6-carboxamide;
(S)-N-(2,3-dihydro-1H-inden-1-yl)-2-(1-ethylpiperidin-4-yl)-7-methylbenzo[d]thiazole-6-carboxamide;
2-(1-ethylpiperidin-4-yl)-7-methyl-N-(tetrahydro-2H-pyran-4-yl)benzo[d]thiazole-6-carboxamide;
(S)-N-(2,3-dihydro-1H-inden-1-yl)-5-methoxy-2-(piperidin-4-yl)benzo[d]thiazole-6-carboxamide;
N-cyclopentyl-5-methoxy-2-(piperidin-4-yl)benzo[d]thiazole-6-carboxamide;
5-methoxy-2-(piperidin-4-yl)-N-(tetrahydro-2H-pyran-4-yl)benzo[d]thiazole-6-carboxamide;
(S)-N-(chroman-4-yl)-5-methoxy-2-(piperidin-4-yl)benzo[d]thiazole-6-carboxamide;
(S)-N-(2,3-dihydro-1H-inden-1-yl)-4-methyl-2-(piperazin-1-yl)benzo[d]thiazole-6-carboxamide;
(S)-N-(2,3-dihydro-1H-inden-1-yl)-2-(4-ethylpiperazin-1-yl)-4-methylbenzo[d]thiazole-6-carboxamide;
N-cyclobutyl-4-methyl-2-(piperazin-1-yl)benzo[d]thiazole-6-carboxamide;
N-cyclobutyl-4-methyl-2-(4-methylpiperazin-1-yl)benzo[d]thiazole-6-carboxamide;
N-cyclopentyl-4-methyl-2-(piperazin-1-yl)benzo[d]thiazole-6-carboxamide;
N-cyclopentyl-2-(4-ethylpiperazin-1-yl)-4-methylbenzo[d]thiazole-6-carboxamide;
N-cyclopentyl-4-methyl-2-(4-methylpiperazin-1-yl)benzo[d]thiazole-6-carboxamide;
4-methyl-2-(piperazin-1-yl)-N-(tetrahydro-2H-pyran-4-yl)benzo[d]thiazole-6-carboxamide;
2-(4-ethylpiperazin-1-yl)-4-methyl-N-(tetrahydro-2H-pyran-4-yl)benzo[d]thiazole-6-carboxamide;
(S)-N-(chroman-4-yl)-4-methyl-2-(piperazin-1-yl)benzo[d]thiazole-6-carboxamide;
(S)-N-(chroman-4-yl)-2-(4-ethylpiperazin-1-yl)-4-methylbenzo[d]thiazole-6-carboxamide;
N-cyclopentyl-5-methyl-2-(piperazin-1-yl)benzo[d]thiazole-6-carboxamide;
N-cyclopentyl-2-(4-ethylpiperazin-1-yl)-5-methylbenzo[d]thiazole-6-carboxamide;
(S)-N-(chroman-4-yl)-5-methyl-2-(piperazin-1-yl)benzo[d]thiazole-6-carboxamide;
(S)-N-(chroman-4-yl)-2-(4-ethylpiperazin-1-yl)-5-methylbenzo[d]thiazole-6-carboxamide;
N-cyclopentyl-7-methyl-2-(piperazin-1-yl)benzo[d]thiazole-6-carboxamide;
N-cyclopentyl-2-(4-ethylpiperazin-1-yl)-7-methylbenzo[d]thiazole-6-carboxamide;
(S)-N-(chroman-4-yl)-7-methyl-2-(piperazin-1-yl)benzo[d]thiazole-6-carboxamide;
(S)-N-(chroman-4-yl)-2-(4-ethylpiperazin-1-yl)-7-methylbenzo[d]thiazole-6-carboxamide;
N-cyclopentyl-4-methoxy-2-(piperazin-1-yl)benzo[d]thiazole-6-carboxamide;
N-cyclopentyl-2-(4-ethylpiperazin-1-yl)-4-methoxybenzo[d]thiazole-6-carboxamide;
(S)-N-(chroman-4-yl)-4-methoxy-2-(piperazin-1-yl)benzo[d]thiazole-6-carboxamide;
(S)-N-(2,3-dihydro-1H-inden-1-yl)-5-methoxy-2-(piperazin-1-yl)benzo[d]thiazole-6-carboxamide;
N-cyclopentyl-5-methoxy-2-(piperazin-1-yl)benzo[d]thiazole-6-carboxamide;
N-cyclopentyl-2-(4-ethylpiperazin-1-yl)-5-methoxybenzo[d]thiazole-6-carboxamide;
5-methoxy-2-(piperazin-1-yl)-N-(tetrahydro-2H-pyran-4-yl)benzo[d]thiazole-6-carboxamide;
2-(4-ethylpiperazin-1-yl)-5-methoxy-N-(tetrahydro-2H-pyran-4-yl)benzo[d]thiazole-6-carboxamide;
(S)-N-(chroman-4-yl)-5-methoxy-2-(piperazin-1-yl)benzo[d]thiazole-6-carboxamide;
(S)-N-(chroman-4-yl)-4,7-dimethyl-2-(piperidin-4-yl)benzo[d]thiazole-6-carboxamide;
4-chloro-N-cyclopentyl-2-(1-methylpiperidin-4-yl)benzo[d]thiazole-6-carboxamide;
4-cyclopropyl-N-isopropyl-2-(piperidin-4-yl)benzo[d]thiazole-6-carboxamide;
N-cyclobutyl-4-cyclopropyl-2-(1-methylpiperidin-4-yl)benzo[d]thiazole-6-carboxamide;
4-chloro-N-cyclobutyl-2-(piperazin-1-yl)benzo[d]thiazole-6-carboxamide;
4-chloro-N-cyclopentyl-2-(4-(3-hydroxypropyl)piperazin-1-yl)benzo[d]thiazole-6-carboxamide;
2-(3,8-diazabicyclo[3.2.1]octan-8-yl)-N-cyclopentyl-7-methoxybenzo[d]thiazole-6-carboxamide;
2-(3,8-diazabicyclo[3.2.1]octan-8-yl)-4-bromo-N-cyclopentylbenzo[d]thiazole-6-carboxamide;
2-(3,8-diazabicyclo[3.2.1]octan-8-yl)-4-cyano-N-isopropylbenzo[d]thiazole-6-carboxamide;
2-(3,8-diazabicyclo[3.2.1]octan-8-yl)-N-cyclopentyl-4-(1-hydroxypropan-2-yl)benzo[d]thiazole-6-carboxamide;
2-(3,8-diazabicyclo[3.2.1]octan-8-yl)-N-cyclopentyl-4-(2-hydroxyethyl)benzo[d]thiazole-6-carboxamide;
   and pharmaceutically acceptable salts thereof.

A compound of Formula Z described herein may be selected from: (2-(piperazin-1-yl)benzo[d]thiazol-6-yl)(pyrrolidin-1-yl)methanone; (2-(piperidin-4-yl)benzo[d]thiazol-6-yl)(pyrrolidin-1-yl)methanone; morpholino(2-(piperidin-4-yl)benzo[d]thiazol-6-yl)methanone;
and pharmaceutically acceptable salts thereof.

Further aspects and embodiments are as set out in the following numbered clauses.

Clause 1. A compound of Formula I

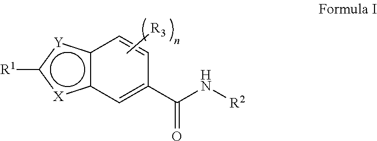

or a pharmaceutically acceptable salt or derivative thereof, wherein:
   one of X and Y is S and the other is N;
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and
   n is 0, 1,2 or 3;
for use in the treatment or prevention of a disease or disorder that can be ameliorated by activation of long isoforms of PDE4.

Clause 2. The compound, or a pharmaceutically acceptable salt or derivative thereof, for use of Clause 1, wherein R¹ is a 5- to 6-membered saturated, monocyclic ring containing at least 1 ring N heteroatom and optionally a ring O heteroatom; a 5- to 6-membered aromatic, monocyclic ring containing 1 or 2 ring N heteroatoms; or a 7- to 8-membered saturated, bridged ring containing 1 or 2 ring N heteroatoms; and wherein R¹ is optionally substituted with 1, 2 or 3 R⁴.

Clause 3. The compound, or a pharmaceutically acceptable salt or derivative thereof, for use of Clause 1 or 2, wherein R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴.

Clause 4. The compound, or a pharmaceutically acceptable salt or derivative thereof, for use of any preceding Clause, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein the Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, (C1-4)alkoxy or OH.

Clause 5. The compound, or a pharmaceutically acceptable salt or derivative thereof, for use of any preceding Clause, wherein R² is:
(i) (C5-6)cycloalkyl fused to a phenyl ring;
(ii) a 5- to 6-membered non-aromatic heterocycle containing one ring O heteroatom, optionally fused to a phenyl ring; or
(iii) (C4-6)cycloalkyl;
   wherein R² is optionally substituted with 1 or more R⁵.

Clause 6. The compound, or a pharmaceutically acceptable salt or derivative thereof, for use of any preceding Clause, wherein R² is a group of formula

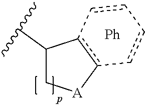

wherein A is O or CH₂; p is 1, 2 or 3; Ph is an optionally present, fused phenyl ring, and wherein R² is optionally substituted with 1 substituent; optionally wherein when A is O, p is 2 or when A is CH₂, p is 1 or 2.

Clause 7. The compound, or a pharmaceutically acceptable salt or derivative thereof, for use of any preceding Clause, wherein each R³ is independently —CH₃ or —OCH₃.

Clause 8. The compound, or a pharmaceutically acceptable salt or derivative thereof, for use of any preceding Clause, wherein n is 0, 1 or 2, preferably 0 or 1.

Clause 9. The compound, or a pharmaceutically acceptable salt or derivative thereof, for use of any preceding Clause, wherein the compound is:

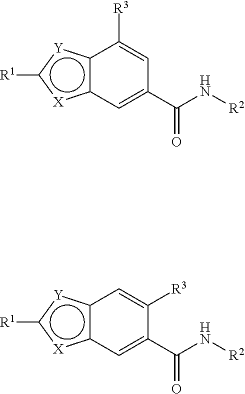

or a pharmaceutically acceptable salt or derivative thereof.

Clause 10. The compound, or a pharmaceutically acceptable salt or derivative thereof, for use of any preceding Clause, wherein X is S and Y is N.

Clause 11. The compound, or a pharmaceutically acceptable salt or derivative thereof, for use of any preceding Clause, wherein
X is S and Y is N;
R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 R⁵;
R³, where present, is methyl;
R⁴, where present, is (C1-6)alkyl optionally substituted with OH;
R⁵, where present, is OH; and
n is 0 or 1.

Clause 12. A compound of Formula II

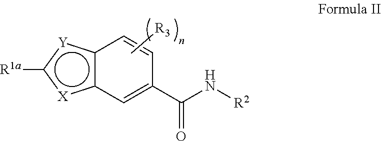

or a pharmaceutically acceptable salt or derivative thereof, wherein:
   one of X and Y is S and the other is N;
   R^{1a} is a 4- to 10-membered non-aromatic, monocyclic, bridged or bicyclic ring containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and
   n is 0, 1,2 or 3;
   wherein when R^{1a} is 4-cyclopentylpiperazin-1-yl, 4-cyclopropylpiperazin-1-yl or 4-isopropylpiperazin-1-yl and n is 0, R² is not unsubstituted, straight chain or branched (C3-6)alkyl or unsubstituted (C3-8)cycloalkyl.

Clause 13. The compound or a pharmaceutically acceptable salt or derivative thereof of Clause 12, wherein:
a) each R⁴ is independently halogen, CN, OH, (C1-2)alkyl, (C1-6)alkoxy or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy; and/or
b) n is 1, 2 or 3; and/or
c) R² is (C5-7)cycloalkyl, fused to a 6-membered aromatic or heteroaromatic ring that contains 0, 1 or 2 ring N atoms; a 5- to 7-membered non-aromatic heterocycle containing one ring O heteroatom, optionally fused to a 6-membered aromatic or heteroaromatic ring that contains 0, 1 or 2 ring N atoms; or CH₂Ar, where Ar is a 6-membered aromatic or heteroaromatic ring that contains 0, 1 or 2 ring N atoms; and wherein R² is optionally substituted with 1 or more R⁵.

Clause 14. The compound or a pharmaceutically acceptable salt or derivative thereof of Clause 12 or 13, wherein R^{1a} is a 5- to 6-membered saturated, monocyclic ring containing at least 1 ring N heteroatom and optionally a ring O heteroatom; or a 7- to 8-membered saturated, bridged ring containing 1 or 2 ring N heteroatoms, and wherein R^{1a} is optionally substituted with 1, 2 or 3 R⁴.

Clause 15. The compound, or a pharmaceutically acceptable salt or derivative thereof, of any of Clauses 12-14, wherein R^{1a} is a 6-membered saturated monocyclic ring containing 1 or 2 ring N heteroatoms, wherein R^{1a} is optionally substituted with 1 R⁴.

Clause 16. The compound, or a pharmaceutically acceptable salt or derivative thereof, of any of Clauses 12-15, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered heterocycle is optionally substituted with 1 to 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, OH or (C1-4)alkoxy.

Clause 17. The compound, or a pharmaceutically acceptable salt or derivative thereof, of any of Clauses 12-16, wherein R² is:
(i) (C5-6)cycloalkyl fused to a phenyl ring;
(ii) a 5- to 6-membered non-aromatic heterocycle containing one ring O heteroatom, optionally fused to a phenyl ring; or
(iii) (C4-6)cycloalkyl;
   wherein R² is optionally substituted with 1 or more R⁵.

Clause 18. The compound, or a pharmaceutically acceptable salt or derivative thereof, of any of Clauses 12-17, wherein R² is a group of formula

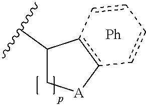

wherein A is O or CH₂; p is 1, 2 or 3; Ph is an optionally present, fused phenyl ring, and wherein R² is optionally substituted with 1 substituent; optionally wherein when A is O, p is 2 or when A is CH₂, p is 1 or 2.

Clause 19. The compound, or a pharmaceutically acceptable salt or derivative thereof, of any of Clauses 12-18, wherein
X is S and Y is N;
R^{1a} is a 6-membered saturated monocyclic ring containing 1 or 2 ring N heteroatoms, wherein at least 1 ring N heteroatom is not at the point of attachment of R¹¹, and wherein R^{l1} is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 R⁵;
R³, where present, is methyl;
R⁴, where present, is (C1-6)alkyl optionally substituted with OH;
R⁵, where present, is OH; and
n is 0 or 1.

Clause 20. A compound of Formula III

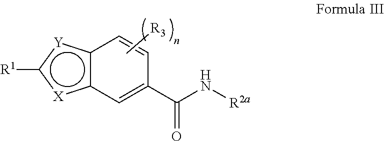

or a pharmaceutically acceptable salt or derivative thereof, wherein:
   one of X and Y is S and the other is N;
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R^{2a} is
   (i) (C5-7)cycloalkyl fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   and wherein R^{2a} is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
   each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and
   n is 0, 1,2 or 3.

Clause 21. The compound, or a pharmaceutically acceptable salt or derivative thereof, of any of Clauses 12-20, wherein n is 0, 1 or 2, optionally n is 0 or 1.

Clause 22. A compound of Formula IV

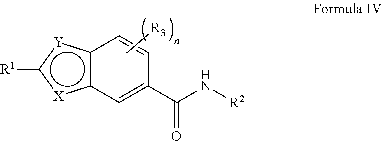

or a pharmaceutically acceptable salt or derivative thereof, wherein:
   one of X and Y is S and the other is N;
   R¹ is a 4- to 10-membered monocyclic, bridged or bicyclic ring containing at least 1 ring N heteroatom and optionally a ring O heteroatom, and wherein R¹ is optionally substituted with 1 or more R⁴;
   R² is
   (i) (C5-7)cycloalkyl, fused to a 6-membered aromatic or heteroaromatic ring that contains 0, 1 or 2 ring N atoms;
   (ii) a 5- to 7-membered non-aromatic heterocycle containing one ring O heteroatom, optionally fused to a 6-membered aromatic or heteroaromatic ring that contains 0, 1 or 2 ring N atoms;
   (iii) CH₂Ar, where Ar is a 6-membered aromatic or heteroaromatic ring that contains 0, 1 or 2 ring N atoms; or
   (iv) a (C3-8)alkyl group that may be straight chain, branched or cyclic, or a combination thereof;
   and wherein R² is optionally substituted with 1 or more R⁵;
   each R³ is independently (C1-6)alkyl, (C1-6)alkoxy, CN or halogen, the (C1-6)alkyl and (C1-6)alkoxy being optionally substituted by 1 or more halogen;
   each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
   each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and
   n is 1,2 or 3.

Clause 23. The compound, or a pharmaceutically acceptable salt or derivative thereof, of Clause 22, wherein the compound is a compound of Formula IVa or IVb

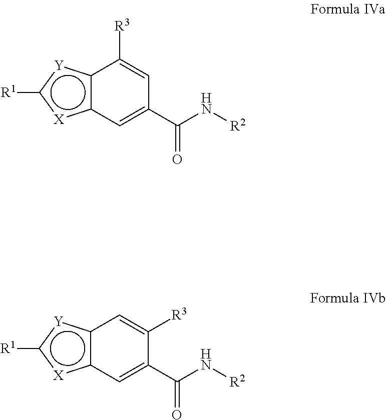

or a pharmaceutically acceptable salt or derivative thereof.

Clause 24. The compound, or a pharmaceutically acceptable salt or derivative thereof, of any of Clauses 12-23, wherein each R³ is independently —CH₃ or —OCH₃.

Clause 25. The compound, or a pharmaceutically acceptable salt or derivative thereof, of any of Clauses 12-24, wherein R² or R^{2a} is:
(i) (C5-7)cycloalkyl fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro.

Clause 26. The compound, or a pharmaceutically acceptable salt or derivative thereof, of any of Clauses 12-25, wherein R² or R^{2a} is:
(i) (C5-6)cycloalkyl fused to a phenyl ring; or
(ii) a 5- to 6-membered non-aromatic heterocycle containing one ring O heteroatom, optionally fused to a phenyl ring;
   wherein R² or R^{2a} is optionally substituted.

Clause 27. The compound, or a pharmaceutically acceptable salt or derivative thereof, of any of Clauses 12-26, wherein R² or R^{2a} is a group of formula

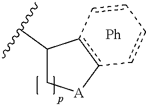

wherein A is O or CH₂; p is 1, 2 or 3; Ph is an optionally present, fused phenyl ring, and wherein R² or R^{2a} is optionally substituted with 1 substituent and wherein when A is CH₂, Ph is present; optionally wherein when A is O, p is 2 or when A is CH₂, p is 1 or 2.

Clause 28. The compound, or a pharmaceutically acceptable salt or derivative thereof, of Clause 20, wherein
X is S and Y is N;
R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴;
R^{2a} is
(i) (C5-6)cycloalkyl fused to a phenyl ring; or
(ii) a 5- to 6-membered non-aromatic heterocycle containing one ring O heteroatom, optionally fused to a phenyl ring;
and wherein R^{2a} is optionally substituted with 1 R⁵;
R⁴, where present, is (C1-6)alkyl optionally substituted with OH;
R⁵, where present, is OH; and
n is 0.

Clause 29. The compound or a pharmaceutically acceptable salt or derivative thereof, of any of Clauses 20 to 28, wherein R¹ is a 5- to 6-membered saturated, monocyclic ring containing at least 1 ring N heteroatom and optionally a ring O heteroatom; a 5- to 6-membered aromatic, monocyclic ring containing 1 or 2 ring N heteroatoms; or a 7- to 8-membered saturated, bridged ring containing 1 or 2 ring N heteroatoms, and wherein R¹ is optionally substituted with 1, 2 or 3 R⁴.

Clause 30. The compound, or a pharmaceutically acceptable salt or derivative thereof, of any of Clauses 20 to 29, wherein R¹ is a 6-membered saturated or aromatic monocyclic ring containing 1 or 2 ring N heteroatoms, wherein at least 1 ring N heteroatom is not at the point of attachment of R¹, and wherein R¹ is optionally substituted with 1 R⁴.

Clause 31. The compound, or a pharmaceutically acceptable salt or derivative thereof, of any of Clauses 12-30, wherein X is S and Y is N.

Clause 32. A pharmaceutical composition comprising a compound or a pharmaceutically acceptable salt or derivative as defined in any of Clauses 1-31, and a pharmaceutically acceptable excipient.

Clause 33. A compound or pharmaceutically acceptable salt or derivative of any of Clauses 12-31 for use in therapy.

Clause 34. A compound or pharmaceutically acceptable salt or derivative of any of Clauses 12-31 or a pharmaceutical composition of Clause 32 for use in the treatment or prevention of a disease or disorder that can be ameliorated by activation of long isoforms of PDE4.

Clause 35. The compound or pharmaceutically acceptable salt or derivative for use of any of Clauses 1 to 11 or the compound or pharmaceutically acceptable salt or derivative or pharmaceutical composition for use of Clause 34 in the treatment or prevention of a disease or disorder mediated by excessive intracellular cyclic AMP signalling.

Clause 36. The compound or pharmaceutically acceptable salt or derivative or pharmaceutical composition for use of Clause 35 wherein the excessive intracellular cyclic AMP signalling is caused by:
a. excessive hormone levels produced by an adenoma.
b. a gain-of-function gene mutation in a G-protein coupled receptor (GPCR);
c. an activating mutation in the GNAS1 gene, which encodes the a-subunit of the G-protein Gₛ; or
d. a bacterial toxin.

Clause 37. The compound or pharmaceutically acceptable salt or derivative or pharmaceutical composition for use of any of Clauses 1-11 or 34-36, wherein the disease is cancer.

Clause 38. The compound or pharmaceutically acceptable salt or derivative or pharmaceutical composition for use of Clause 37, wherein the cancer is prostate cancer.

Clause 39. The compound or pharmaceutically acceptable salt or derivative or pharmaceutical composition for use of any of Clauses 1-11 or 34-36, wherein the disease is:
a. pituitary adenoma, Cushing's disease, polycystic kidney disease or polycystic liver disease;
b. hyperthyroidism, Jansens's metaphyseal chondrodysplasia, hyperparathyroidism, or familial male-limited precocious puberty;
c. McCune-Albright syndrome;
d. cholera, whooping cough, anthrax, or tuberculosis;
e. HIV, AIDS, or Common Variable Immunodeficiency (CVID);
f. melanoma, pancreatic cancer, leukaemia, prostate cancer, adrenocortical tumours, testicular cancer, primary pigmented nodular adrenocortical diseases (PPNAD), or Carney Complex;
g. autosomal dominant polycystic kidney disease (ADPKD) or autosomal recessive polycystic kidney disease (ARPKD); or
h. maturity onset diabetes of young type 5 (MODY5); or
i. cardiac hypertrophy.

Clause 40. The compound or pharmaceutically acceptable salt or derivative or pharmaceutical composition for use of Clause 39, wherein the disease is:
a. autosomal dominant polycystic kidney disease (ADPKD); or
b. autosomal recessive polycystic kidney disease (ARPKD).

Clause 41. The compound or pharmaceutically acceptable salt or derivative or pharmaceutical composition for use of Clause 39, wherein the disease is hyperparathyroidism.

### Definitions

The term “aromatic ring” refers to an aromatic carbocyclic ring system. The term “heteroaromatic ring” refers to an aromatic ring system wherein one or more of the ring-forming atoms is a heteroatom such as O, S or N. An aromatic ring may be a 6-membered aromatic ring, i.e. a phenyl ring. A heteroaromatic ring may be a 6-membered heteroaromatic ring that contains one to three N atoms or a 5-membered heteroaromatic ring that contains one to three heteroatoms selected from O, S and N. Examples of such 6- or 5-membered heteroaromatic rings include pyridine, pyridazine, pyrazine, pyrimidine, thiophene, furan, thiazole, thiadiazole, oxazole, oxadiazole, imidazole, triazole and their isomers including isothiazole, isothiadiazole, isoxazole and isoxadiazole. In all instances described above, an aromatic ring may be optionally substituted as defined herein.

The term “carbocyclic ring” refers to a ring system with may be saturated, partially unsaturated or aromatic and wherein all ring forming atoms are carbon. The term “heterocyclic ring” refers to a ring system with may be saturated, partially unsaturated or aromatic and wherein one or more of the ring-forming atoms is a heteroatom such as O, S or N. A “non-aromatic carbocyclic or heterocyclic ring” may be saturated or partially unsaturated. Carbocyclic and heterocyclic rings may be bicyclic or multicyclic ring systems, for example bicyclic or multicyclic fused ring systems or bicyclic or multicyclic spiro ring systems or a combination thereof. Each ring within a fused ring system may independently be saturated, partially unsaturated or aromatic. Examples of such fused bicyclic ring systems include indane and chromane. A non-aromatic carbocyclic or heterocyclic ring may include fused ring systems, where for example two rings share two adjacent atoms, bridged ring systems, where for example two rings share three or more adjacent atoms, or spiro ring systems, where for example two rings share one adjacent atom. Examples of fused ring systems include octahydropyrrolo[1,2-a]pyrazine and octahydro-2H-pyrido[1,2-a]pyrazine. Bridged rings may comprise three or more rings. Examples of such bridged ring systems include 2,5-diazabicyclo[2.2.1]heptane, 2,5-diazabicyclo[2.2.2]octane and 3,8-diazabicyclo[3.2.1]octane. Examples of spiro ring systems include spiro[4.3]octane and 2,6-diazaspiro[3.4]octane. In all instances described above, a carbocyclic or heterocyclic ring may be optionally substituted as defined herein.

Where a ring is referred to herein as containing specified ring heteroatoms, it will be appreciated that no further ring heteroatoms are present beyond those specified. A “monocyclic, bridged or bicyclic ring” includes monocyclic rings, bridged ring systems and bicyclic ring systems. A “monocyclic, bridged or bicyclic ring”, unless otherwise defined, may be saturated, partially unsaturated or aromatic. These may be aromatic, heteroaromatic, carbocyclic or heterocyclic rings or combinations thereof. Bicyclic ring systems may include fused and spiro rings.

Unless otherwise defined, the term “alkyl” as used herein refers to a saturated hydrocarbon which may be straight-chain, branched, cyclic or a combination thereof. Alkyl groups include linear, branched or cyclic alkyl groups and hybrids thereof, such as (cycloalkyl)alkyl. The term “(C1-6)alkyl” as used herein means an alkyl group having 1-6 carbon atoms, which may be branched or unbranched and optionally contains a ring. Examples of (C1-6)alkyl include hexyl, cyclohexyl, pentyl, cyclopentyl, butyl, isobutyl, cyclobutyl, tertiary butyl, propyl, isopropyl, cyclopropyl, cyclopropylmethyl, ethyl and methyl. The term “(C1-4)alkyl” as used herein means a branched or unbranched alkyl group having 1-4 carbon atoms, optionally containing a ring. Examples of (C1-4)alkyl include butyl, isobutyl, cyclobutyl, tertiary butyl, propyl, isopropyl, cyclopropyl, cyclopropylmethyl, ethyl and methyl. A (C1-4)alkyl as referenced herein may preferably be a (C1-2)alkyl. Where specified in the formulae above, (C1-4)alkyl may be substituted, for example with 1 to 3 fluoros. A particularly preferred example of a substituted (C1-4)alkyl is trifluoromethyl. Alternatively (C1-4)alkyl may be unsubstituted.

The term “alkylene” as used herein refers to a divalent alkyl group.

The term “cycloalkyl” refers to a cyclic alkyl group, for example cycloheptyl, cyclohexyl, cyclopentyl, cyclobutyl or cyclopropyl. Cycloalkyl may be substituted as defined herein.

The term “alkoxy” means —O-alkyl wherein alkyl has the meaning as defined above. Examples of (C1-4)alkoxy include methoxy, ethoxy, propoxy, isopropoxy, butoxy, isobutoxy and tertiary butoxy. A (C1-4)alkoxy as referenced herein may preferably be a (C1-2)alkoxy. Where specified in the formulae above, (C1-4)alkoxy may be substituted, for example with 1 to 3 fluoros. A particularly preferred example of a substituted (C1-4)alkoxy is trifluoromethoxy. Alternatively, (C1-4)alkoxy may be unsubstituted. In the present invention, alkoxy is attached to the rest of the molecule by the “oxy” moiety.

A group that is referred to herein as being “substituted”, whether preceded by the term “optionally” or not, means that at least one hydrogen present on a group (e.g. a C or N atom) is replaced with a permissible substituent, for example a substituent which upon substitution results in a stable compound, e.g. a compound which does not spontaneously undergo transformation such as by rearrangement, cyclization, elimination or other reaction. Unless otherwise indicated, when more than one substituent is present, the substituent is either the same or different at each occurrence. Unless otherwise indicated, a “substituted” group has one or more substituents at one or more substitutable positions of the group, and when more than one position in any given structure is substituted, the substituent is either the same or different at each position.

The term “halogen” means F, Cl, Br or I. F and Cl are particularly preferred, with F the most preferred.

Activation of Long PDE4 Isoforms

PDE4 long isoforms have two regulatory regions, upstream conserved region 1 (UCR1) and upstream conserved region 2 (UCR2). These are between the isoform-specific N-terminal portion and the catalytic domain. The UCR1 domain is missing in the short forms, whereas the super-short forms not only lack UCR1, but also have a N-terminal truncated UCR2 domain (Houslay, M. D., Schafer, P. and Zhang, K. Drug Discovery Today 10: 1503-1519, 2005).

There are four PDE4 families, PDE4A, PDE4B, PDE4C and PDE4D. The present invention concerns compounds that are capable of activating one or more of the long isoforms from one or more of these four families. The long isoform PDE4 may therefore be long isoform PDE4A, long isoform PDE4B, long isoform PDE4C or long isoform PDE4D. For the avoidance of doubt, a long isoform PDE4 contains a UCR1 region. In some embodiments, a long isoform PDE4 as referred to herein is human. UCR1 is conserved within mammalian species (Houslay, MD, Sullivan, M and Bolger GB Adv. Pharmacol. 44: 225-342, 1998), so in other embodiments, the long isoform PDE4 can be from a non-human mammal.

Without wishing to be bound by theory, the compounds described herein may act as PDE4 long form activators. The compounds described herein are small molecules that are believed to bind directly to PDE4 long forms and induce structural changes that increase, stabilise, uncover and/or maintain the catalytic activity of these enzymes. Without wishing to be bound by theory, the activation of PDE4 long forms by PDE4 long form activators may be sensitive to the regulatory status of the enzyme, including post-translational modifications (such as phosphorylation) or the adoption of protein-protein complexes associated with a particular physiological localisation or with a cellular or biochemical assay context. PDE4 long form activators may manifest activation of the enzyme in one or more states but not necessarily all states.

In the field of pharmacology, and as used herein, a small molecule is defined as a low molecular weight organic compound that may serve as a regulator of biological processes. Preferred small molecule activators according to the present invention have a molecular weight of less than or equal to 700 Daltons. This allows for the possibility to rapidly diffuse across cell membranes and reach intracellular sites of action (Veber, D. F. et al., J. Med. Chem. 45: 2615-2623, 2002). Especially preferred small molecule activators according to the present invention have molecular weights of greater than or equal to 250 Daltons and less than or equal to 500 Daltons (Lipinski, C. A. Drug Discovery Today: Technologies 1: 337-341, 2004).

One suitable method of detecting whether or not a compound is capable of serving as an activator of a PDE4 long form is using a two-step radio-assay procedure described in Experiment 1. In summary, the method involves incubating a PDE4 long form with a test small molecule activator, together with [³H]-labelled cAMP to assess alterations in the breakdown of cAMP to the 5′-adenosine monophosphate (5′-AMP) product. A sample of the reaction mixture from such an incubation is subsequently treated with snake venom 5′-nucleotidase to allow conversion of the nucleotide [³H]-labelled 5′-AMP to the uncharged nucleoside [³H]-labelled adenosine, which can be separated and quantified to assess PDE4 activity and the effect of the test compound (Thompson, W. J. and Appleman, M. M. Biochemistry 10: 311-316, 1971, with some modifications as described in: Marchmont, R. J. and Houslay, M. D. Biochem J. 187: 381-92, 1980).

Using the above assay procedure, as described in detail in Experiment 1, preferred compounds described herein may produce an increase in the background activity of one or more PDE4 long forms of more than 30% at a test compound concentration of 100 micromolar or less. Especially preferred compunds described herein may produce an increase in the background activity of one or more PDE4 long forms of more than 30% at a test compound concentration of 10 micromolar, or less, for example 3 micromolar.

The compounds described herein may be selective for the long form of the PDE4 enzyme and, as such, do not act or act to a lesser extent as activators of the short or super-short isoforms of the PDE4 enzyme. The short or super-short isoform PDE4 may be short or super-short isoform PDE4A, short or super-short isoform PDE4B, short or super-short isoform PDE4C, or short or super-short isoform PDE4D. For the avoidance of doubt, short and super-short isoforms of PDE4 lack a UCR1 domain. Super-short isoforms are characterised by a truncated UCR2 domain and lack of a UCR1 domain. The short or super-short isoform PDE4 is, for example, human, but may also be from other mammalian species (where UCR2 is conserved, see Houslay, MD, Sullivan, M and Bolger GB Adv. Pharmacol. 44: 225-342, 1998).

Under the same assay conditions, as described in Experiment 1, the compounds described herein may produce a less than 30% increase in the background activity of the short or super-short forms of the PDE4A, PDE4B, PDE4C or PDE4D enzymes at a test compound concentration of 100 micromolar, or less.

Compounds described herein may therefore provide a positive result in an assay for activation of a long form PDE4 and a negative result in an assay for activation of a short form (or super-short form) of PDE4.

PDE4 long isoforms include those now known as PDE4A4, PDE4A4/5, PDE4A5, PDE4A8, PDE4A10, PDE4A11, PDE4B1, PDE4B3, PDE4B4, PDE4C1, PDE4C2, PDE4C3, PDE4C4, PDE4D3, PDE4D4, PDE4D5, PDE4D7, PDE4D8, PDE4D9 and PDE4D11. Further long isoforms may be or have already been identified or called by different nomenclature from any of the four PDE4 sub-families.

PDE4 short and super-short isoforms include PDE4A1, PDE4B2, PDE4B5, PDE4D1, PDE4D2, PDE4D6 and PDE4D10. Further short and super-short isoforms may be or have already been identified or called by different nomenclature from any of the four PDE4 sub-families.

The Examples below exemplify activity of compounds described herein in an assay for activation of the human PDE4D5 and PDE4C3 long isoforms and a lack of activity in an assay for activation of the human PDE4B2 short isoform. Details of these isoforms and a number of the other known isoforms, including GenBank accession numbers, are provided in Tables A to D immediately below.

TABLE A
Examples of known PDE4A Isoforms

| | | | Calculated | |
|---|---|---|---|---|
| | | | molecular | |
| Isoform | Species | Accession | weight (kDa) | Type |
| PDE4A1 | Human | NM_006202 | 73 | Short |
| PDE4A1 | Rodent | L27062 | 68 | Short |
| PDE4A4* | Human | L20965 | 98 | Long |
| PDE4A5 | Rodent | L27057 | 93 | Long |
| PDE4A7** | Human | U18088 | 37 | Dead-Short |
| PDE4A8 | Human | AY593872 | 96 | Long |
| PDE4A8 | Rodent | L36467 | 85 | Long |
| PDE4A10 | Human | AF073745 | 91 | Long |
| PDE4A11 | Human | AY618547 | 95 | Long |

*Note that the PDE4A4B clone is correct while PDE4A4A has a cloning artefact and PDE4A4C is a truncation artefact.
**Note that this species is C- as well as N-terminally truncated

TABLE B
Examples of known PDE4B Isoforms

| | | | Calculated | |
|---|---|---|---|---|
| | | | molecular | |
| Isoform | Species | Accession | weight (kDa) | Type |
| PDE4B1 | Human | NM_001037341.1 | 83 | Long |
| PDE4B2 | Human | NM_001037339.1 | 64 | Short |
| PDE4B3 | Human | NM_001037340 | 83 | Long |
| PDE4B4 | Rodent | AF202733.1 | 75 | Long |
| PDE4B5 | Human | EF595686.1 | 57 | Super-short |

TABLE C
Examples of known PDE4C Isoforms

| | | | Calculated molecular | |
|---|---|---|---|---|
| Isoform | Species | Accession | weight (kDa) | Type |
| PDE4C1 | Human | NM_000923 | 79 | Long |
| PDE4C2 | Human | NM_001098819 | 67 | Long |
| PDE4C3 | Human | NM_001098818 | 76 | Long |
| PDE4C4 | Human | U66346 | 88 | Long |
| PDE4C5 | Human | U66347 | 47 | Partial |
| PDE4C6 | Human | U66348 | 58 | Partial |
| PDE4C7 | Human | U66349 | 48 | Partial |

TABLE D
Examples of known PDE4D Isoforms

| | | | Calculated | |
|---|---|---|---|---|
| | | | molecular | |
| Isoform | Species | Accession | weight (kDa) | Type |
| PDE4D1 | Human | NM_001197222 | 66 | Short |
| PDE4D2 | Human | NM_001197221 | 58 | Super-short |
| PDE4D3 | Human | NM_006203 | 76 | Long |
| PDE4D4 | Human | NM_001104631 | 91 | Long |
| PDE4D5 | Human | NM_001197218 | 84 | Long |
| PDE4D6 | Human | NM_001197223 | 59 | Super-short |
| PDE4D7 | Human | NM_001165899 | 85 | Long |
| PDE4D8 | Human | NM_001197219 | 78 | Long |
| PDE4D9 | Human | NM_001197220 | 77 | Long |
| PDE4D10 | Rodent | DQ665896.1 | 58 | Super-short |
| PDE4D11 | Rodent | EU489880.1 | 79 | Long |

* PDE4D8 was originally called PDE4D6 in the literature

Reduction of cAMP Levels

Without wishing to be bound by theory, the compounds described herein may function by reducing cAMP levels in one or more intracellular compartments. The PDE4 long form activators described herein may thus provide a means to regulate certain cellular processes that are dependent upon cAMP. Excessive intracellular cAMP signalling mediates a number of diseases and disorders. Therefore, the compounds described herein are expected to be of utility for the treatment of diseases associated with abnormally elevated cAMP levels, increased cAMP-mediated signalling and/or reduced cAMP elimination, enzymatic or otherwise (e.g. via efflux). The treatment is typically of a human, but may also be of a non-human animal, such as a non-human mammal (e.g. veterinary treatment).

In one aspect, the present invention provides a compound described here (i.e. a small molecule activator of a PDE4 long form), for use in a method for the treatment or prevention of disorders where a reduction of second messenger responses mediated by cyclic 3′,5′-adenosine monophosphate (cAMP) is required.

For example, gain-of-function gene mutations in proteins involved in driving cAMP signalling upstream of adenylyl cyclase, such as GPCRs and Gsa, can lead to abnormal excessive cAMP activity with pathological consequences (Lania A, Mantovani G, Spada A. Ann Endocrinol (Paris). 73: 73-75, 2012.; Thompson, M. D. et al., Methods Mol. Biol. 448: 109-137, 2008; Weinstein L S, Liu J, Sakamoto A, Xie T, Chen M. Endocrinology. 145: 5459-5464, 2004; Lania A, Mantovani G, Spada A. Eur J Endocrinol. 145: 543-559, 2001). PDE4 long form activators described herein, possessing the ability to accelerate the termination of cAMP action, would therefore be expected to be effective in the treatment, prevention or partial control of diseases characterised by undesirably high cAMP levels, or activity, as detailed below.

The treatment or prevention described herein may be treatment or prevention of a disease or disorder that can be ameliorated by activation of long isoforms of PDE4. The treatment or prevention described herein may be treatment or prevention of a disease or disorder mediated by excessive intracellular cAMP signalling. In these diseases, a reduction of second messenger responses mediated by cyclic 3′,5′-adenosine monophosphate (cAMP) should provide a therapeutic benefit.

Diseases Ameliorated by Activation of Long Isoforms of PDE4 or Characterised by Elevated cAMP Levels

Hyperthyroidism Stimulation of the thyroid-stimulating hormone (TSH) receptor (TSHR) leads to increased generation and release of thyroid hormones, thyroxine and triiodothyronine, through a cAMP-dependent signalling mechanism involving Gsa-mediated activation of adenylyl cyclase. Gain-of-function mutations in the TSHR have been reported to be involved in the development of hyperthyroidism (Duprez, L. et al., Nat. Genet. 7: 396-401, 1994; Biebermann, H. et al., J. Clin. Endocrinol. Metab. 86: 4429-4433, 2001; Karges, B. et al., J. Endocrinol. 186: 377-385, 2005). Activating mutations of both TSHR and Gsa have also been found in goitre and thyroid adenomas (Arturi, F. et al., Exp. Clin. Endocrinol. Diabetes 106: 234-236, 1998). The increased cAMP activity in thyroid adenomas, as a result of the activating TSHR or Gsa mutations, has been reported to produce a protective adaptive increase in PDE4 activity to counteract abnormal rise in cAMP levels and signal transduction (Persani, L. et al., J. Clin. Endocrinol. Metab. 85: 2872-2878, 2000).

The most common cause of hyperthyroidism is Graves' disease, an autoimmune disorder in which antibodies mimic TSH action at the TSHR, leading to excessive cAMP activity in thyroid follicle cells and consequently a state of hyperthyroidism.

PDE4 long form activators described herein are therefore expected to be effective in the treatment, prevention or partial control of hyperthyroidism. In one embodiment, the hyperthyroidism is associated with Graves' disease.

Jansens's Metaphyseal Chondrodysplasia

Jansens's Metaphyseal Chondrodysplasia (JMC) is a very rare disease resulting from gain-of-function mutations of the parathyroid hormone (PTH) receptor 1 (PTHR1) (Thompson, M. D. et al., Methods Mol. Biol. 448: 109-137, 2008). The constitutive activation of the PTHR1 which couples to adenylyl cyclase as effector is associated with excessive cAMP signalling primarily in bone and kidney, leading to dysregulation of ion homeostasis characterised by hypercalcemia and hypophosphatemia (Calvi, L. M. and Schipani, E. J. Endocrinol. Invest. 23: 545-554, 2000) and developmental (e.g. short stature) and physical (e.g. protruding eyes) abnormalities. PDE4 long form activators described herein are therefore expected to be effective in the treatment, prevention or partial control of JMC.

Hyperparathyroidism

Hyperparathyroidism (HPT) is characterized by excessive secretion from the parathyroid gland of PTH, which regulates plasma calcium and phosphate concentrations via PTHR1 receptors in the kidney, bone and GI tract. The resulting excessive stimulation of these receptors causes disruption of plasma ion homeostasis with patients showing hypercalcemia and hypophosphatemia. Primary HPT is driven by parathyroid gland hyperplasia or dysfunction, whereas secondary HPT is associated with underlying medical conditions, predominantly chronic renal disease. Left untreated, HPT causes a variety of debilitating symptoms and can become life-threatening.

By acting to down-regulate excessive cAMP generated by sustained PTH signalling, PDE4 long form activators described herein are expected to be effective in the treatment, prevention or partial control of hyperparathyroidism.

Familial Male Precocious Puberty (Testotoxicosis)

Familial male-limited precocious puberty (FMPP), also known as familial sexual precocity or gonadotropin-independent testotoxicosis, is a disorder in which boys generally develop signs of precocious puberty in early childhood.

The spinal length in boys may be short due to a rapid advance in epiphyseal maturation. FMPP is an autosomal dominant condition with constitutively activating mutations in the luteinizing hormone (LH) receptor, which leads to increased cAMP production, associated with Leydig cell hyperplasia and low sperm cell count (Latronico, A. C. et al., J Clin. Endocrinol. Metab. 80: 2490-2494, 1995; Kosugi, S. et al., Hum. Mol. Genet. 4: 183-188, 1995). PDE4 long form activators described herein are therefore expected to be effective in the treatment, prevention or partial control of FMPP.

Pituitary Adenomas and Cushing's Disease

Non-cancerous tumours of the pituitary gland are collectively referred to as pituitary adenomas and can lead to hypersecretion of adenohypophyseal hormones (e.g. growth hormone, thyroid stimulating hormone, luteinizing hormone, follicle stimulating hormone and adrenocorticotrophic hormone), which exert their action through GPCRs coupled to Gs and cAMP generation. Thus pituitary adenomas can lead to a state of enhanced cAMP mediated signalling in a variety of endocrine tissues which can precipitate a number of hormonal disorders such as acromegly (mainly due to excess growth hormone secretion), Cushing's disease (due to overproduction of adrenocorticotrophic hormone (ACTH) and the subsequent hypercortisolemia) and/or general hyperpituitarism (associated with excess release of multiple anterior pituitary hormones). Current treatment options for pituitary adenomas include treatment with dopamine receptor agonists, which reduce tumour size and lower pituitary hormonal output through a mechanism involving lowering of intracellular cAMP levels. PDE4 long form activators described herein may also be expected to attenuate the pathological effects of pituitary hormones in their target tissues, such as the adrenal glands.

In Cushing's disease, pituitary adenoma related overproduction of ACTH can lead to hypercortisolemia through an overactivation of melanocortin 2 receptor (MC2) and subsequent cAMP mediated stimulation of steroidogenesis and release of cortisol from the adrenal cortex (Tritos, N. A. and Biller, B. M. Discov. Med. 13: 171-179, 2012). PDE4 long form activators described herein are therefore expected to be effective in the treatment, prevention or partial control of Cushing's disease.

Polycystic Kidney Disease

Polycystic kidney disease (PKD) is a genetic disorder of the kidneys characterised by development of pathological cysts, which damage renal structure and compromise kidney function (Takiar, V. and Caplan, M. J. Biochim. Biophys. Acta. 1812: 1337-1343, 2011; Masoumi, A. et al., Drugs 67: 2495-2510, 2007). There are two types of PKD: autosomal dominant polycystic kidney disease (ADPKD) and autosomal recessive polycystic kidney disease (ARPKD). ADPKD affects between 0.1% and 0.2% of the population worldwide and is characterized by progressive cyst development and enlarged kidneys. Approximately 50% of people with this disease will develop end stage kidney disease, usually between 40 and 70 years of age and require dialysis or kidney transplantation. ARPKD affects around 1:20,000 live births and is typically identified in the first few weeks after birth. Pulmonary hypoplasia results in a 30-50% death rate in neonates with ARPKD.

Defects in two genes are thought to be responsible for ADPKD. In around 85% of patients, development of ADPKD can be linked to mutations in the gene PKD1, encoding polycystin-1 (PC-1); in around 15% of patients mutations in PKD2, encoding polycystin-2 (PC-2) are implicated. Cyclic AMP has been identified as an important stimulus for proliferation and cyst expansion in polycystic kidney cells but not in normal human kidney cells (Yamaguchi, T. et al., Kidney Int. 57: 1460-1471, 2000). A considerable body of evidence has now developed to implicate cAMP as an important facilitator of renal cystogenesis (Masoumi, A. et al., Drugs 67: 2495-2510, 2007; Wallace, D. P. Biochim. Biophys. Acta. 1812: 1291-1300, 2011). Consistent with the role of cAMP in cyst formation, agents that lower cAMP levels (e.g. vasopressin V2 receptor antagonists and the somatostatin receptor agonist octreotide) showed efficacy in rodent models of PKD (Torres, V. E. et al., Nat. Med. 10: 363-364, 2004; Gattone, V. H. 2^{nd} et al., Nat. Med. 9: 1323-1326, 2003; Belibi, F. A. and Edelstein, C. L. Expert Opin. Investig. Drugs. 19: 315-328, 2010). In zebrafish embryos, depletion of a cAMP-hydrolysing PDE enzyme subtype, PDE1A, resulted in development of a cystic phenotype, while PDE1A over-expression partially rescued cystic phenotypes resulting from PC2 depletion (Sussman, C. R., Ward, C. J., Leightner, A. C., Smith, J. L., Agarwal, R., Harris, P. C., Torres, V. E. J. Am. Soc. Nephrol. 25: 2222-2230, 2014). Phosphodiesterase activation has been suggested as a therapeutic strategy for PKD treatment (Sun, Y., Zhou, H. and Yang, B-X. Acta Pharmacologica Sinica 32: 805-816, 2011).

PDE4 long form activators described herein are therefore expected to be effective in the treatment, prevention or partial control of polycystic kidney disease.

Polycystic Liver Disease Polycystic liver disease (PLD) is a rare inherited condition associated with hepatic cystogenesis (usually defined when number of cysts exceeds 20), which often occurs in association with ADPKD (Strazzabosco, M. and Somlo, S. Gastroenterology 140: 1855-1859, 2011; Gevers, T. J. and Drenth, J. P. Curr. Opin. Gastroenterol. 27: 294-300, 2010). PLD may have a different genetic pathology when compared to ADPKD, driven by mutated proteins associated with the endoplasmic reticulum and the cilium. Increased cholangiocyte proliferation, neovascularisation and elevated fluid secretion act to drive liver cyst formation through dysregulation of multiple signal transduction pathways, including cAMP-mediated signalling. Elevation of hepatic cAMP levels stimulates cAMP-dependent chloride and fluid secretion in biliary epithelial cells and increases cholangiocyte proliferation (Janssen, M. J. et al., J. Hepatol. 52: 432-440, 2010). Somatostatin, which acts through a Gi-coupled mechanism to lower cAMP levels, reduced cholangiocyte proliferation and fluid secretion (Gong, A. Y. et al., Am. J. Physiol. Cell. Physiol. 284: C1205-1214, 2003). Furthermore, the synthetic somatostatin analogue, octreotide, showed efficacy in an animal model of PLD through a mechanism involving reduction in cAMP signalling (Masyuk, T. V. et al., Gastroenterology 132: 1104-1116, 2007). PDE4 long form activators described herein may therefore be effective in the treatment, prevention or partial control of polycystic liver disease due at least in part to cAMP.

Maturity Onset Diabetes of Young Type 5 (MODY5)

MODY5 is a form of non-insulin-dependent diabetes mellitus associated with renal cysts. It is an autosomal dominant disorder caused by mutations in the gene encoding hepatocyte nuclear factor-1β (HNF-1p). The predominant clinical feature of patients affected by MODY5 is renal dysfunction, frequently diagnosed before the onset of diabetes. In some patients, HNF-1P mutations can result in additional phenotypic features, such as pancreatic atrophy, abnormal liver function and genital tract abnormalities. Studies in mice suggest that the mechanism responsible for renal cyst formation, associated with mutations of HNF-1β, involves a severe defect of the transcriptional activation of PKD2, in addition to effects on uromodulin (UMOD) and PKD1 genes. Down-regulation of PKD1 and PKD2 is associated with cAMP-driven formation of renal cysts (Mancusi, S. et al., J. Nephrol. 26: 207-12, 2013). HNF-1β also binds to the PDE4C promoter and regulates the expression of PDE4C (Ma et al., PNAS 104: 20386, 2007).

PDE4 long form activators described herein are therefore expected to be effective in the treatment, prevention or partial control of the symptoms of MODY5.

Cardiac Hypertrophy, Heart Failure and Arrhythmia Localized regulation and integration of cAMP signalling are important for proper cardiac function and perturbation of this signalling can lead to heart failure. Upon chronic p-adrenergic receptor stimulation, cardiomyocyte hypertrophy is induced via elevated cAMP and activation of its downstream effectors, including PKA and Epac (Wang, L. et al., Cell. Signal. 27: 908-922, 2015 and references therein). Cardiomyocyte hypertrophy increases the risk of heart failure and arrhythmia.

PDE4 long form activators described herein may therefore be effective in the treatment, prevention or partial control of cardiac hypertrophy, heart failure and/or arrhythmia.

Diseases Associated with Increased cAMP-Mediated Signalling

Disorders Associated with Activating Mutations of the Alpha Subunit of the G Protein (GNAS1)

The G-protein Gs acts as a transducer for GPCRs that stimulate adenylyl cyclase activity and exert their biological effects by increasing intracellular cAMP levels. Gs is a heterotrimeric protein composed of a, p and y subunits. Activating mutations in the gene, GNAS1, for the a-subunit have been identified which lead to exaggerated abnormal cAMP signalling in a variety of tissues and give rise to a range of disorders.

McCune-Albright Syndrome

McCune-Albright syndrome (MAS) is a rare genetic disorder typically characterised by three dominating features of precocious puberty, fibrous dysplasia of bone and café au lait lesions. The underlying molecular pathology for MAS involves an activating mutation of the GNAS1 gene (Diaz, A. Danon, M. and Crawford, J. J. Pediatr. Endocrinol. Metab. 20: 853-880, 2007). PDE4 long form activators described herein would therefore be expected to be effective in the treatment, prevention or partial control of disorders associated with activating mutations of GNAS1, including McCune-Albright syndrome.

Amelioration of Toxin-Induced Increases in Adenylyl Cyclase Activity in Infectious Diseases.

Adenylyl cyclase, the enzyme responsible for production of cAMP, is a key biological target thought to be involved in mediating the effects of many bacterial toxins (Ahuja et al., Critical Reviews in Microbiology, 30: 187-196, 2004). These toxins produce their effects by raising cAMP levels through enhancement of host immune cell and/or pathogen related adenylyl cyclase activity. PDE4 long form activators described herein, by reducing cAMP levels, would therefore be expected to be of utility in the treatment or partial control of symptoms of infectious diseases that are associated with elevated cAMP activity. The following are some examples of such infectious diseases:

Cholera

Vibrio cholerae produces cholera toxin, which through adenosine disphosphate ribosylation of the a subunit of Gs leads to host cell adenylyl cyclase activation and cAMP production. Diarrhoea caused by cholera toxin is believed to be a result of excessive cAMP accumulation in the cells of the gastrointestinal tract.

Whooping Cough

Bordetella pertussis is the pathogen responsible for the childhood disease whooping cough. Bordetella pertussis toxin stimulates adenosine disphosphate ribosylation of the a subunit of Gi and indirectly augments cAMP levels in target cells. The bacterium also secretes an invasive adenylyl cyclase, which produces toxic cAMP levels and impairs host immune defence.

Anthrax

Anthrax is caused by Bacillus anthracis and whilst it is primarily an animal disease it can be transmitted to humans through contact. Anthrax infections are associated with widespread oedema, the development of which is thought to be driven by oedema toxin. The latter is an adenylyl cyclase and is activated by host calmodulin to produce abnormally high levels of cAMP that have a toxic effect on host immune cells.

Tuberculosis

Mycobactrium tuberculosis expresses a large and diverse range of adenylyl cyclases, which may play a role in virulence and generation of disease pathology. One adenylyl cyclase subtype, RV0386, has been demonstrated to enter host macrophages and elevate intracellular cAMP to cause toxicity (Agarwal et al., Nature, 460: 98-102, 2009).

PDE4 long form activators described herein may therefore be effective in the treatment, prevention or partial control of infectious diseases such as cholera, whooping cough, anthrax and tuberculosis.

Diseases Dependent Upon Activation of PKA by Elevated cAMP.

In eukaryotes, cAMP activates protein kinase A (PKA), which is also known as cAMP-dependent protein kinase. PKA is normally inactive as a tetrameric holoenzyme, consisting of two catalytic and two regulatory units, with the regulatory units blocking the catalytic centres of the catalytic units. cAMP binds to specific locations on the regulatory units of PKA and causes dissociation between the regulatory and catalytic units, thus activating the catalytic units. The active catalytic units catalyse the transfer of phosphate from ATP to specific residues of protein substrates, which may modulate the function of those protein substrates.

PDE4 long form activation reduces cAMP levels and cAMP mediated activation of PKA. PDE4 long form activators described herein would therefore be expected to be of utility in the treatment or partial control of disorders where inhibitors of PKA show evidence of therapeutic effects.

Disorders that are dependent upon activation of PKA by cAMP may be identified by their response to PKA inhibitors such as Rp-8-Br-cAMPS. Rp-8-Br-cAMPS is an analogue of cAMP that occupies the cAMP binding sites of PKA, preventing its dissociation and activation.

HIV Infection and AIDS

T cells from HIV-infected patients have increased levels of cAMP and are more sensitive to inhibition by Rp-8-Br-cAMPS than are normal T cells. Excessive activation of PKA by cAMP has been associated with the progressive T cell dysfunction in HIV infection (Aandahl, E. M. et al., FASEB J. 12: 855-862, 1998). Furthermore, in vivo administration of Rp-8-Br-cAMPS has been shown to restore T cell responses in retrovirus-infected mice (Nayjib, B. et al., The Open Immunology Journal, 1: 20-24, 2008). PDE4 long form activators described herein are therefore expected to be of utility in the treatment, prevention or partial control of HIV infection and AIDS.

Common Variable Immunodeficiency (CVID)

In vitro administration of Rp-8-Br-cAMPS has been shown to correct impaired secretion of the cytokine IL-10 by T cells from patients with Common Variable Immunodeficiency (CVID) (Holm, A. M. et al., J. Immunol. 170: 5772-5777, 2003). PDE4 long form activators described herein are therefore expected to be of utility in the treatment, prevention or partial control of CVID.

Diseases Dependent Upon Activation of Either or Both of Epac1 and Epac2 by Elevated cAMP.

In addition to PKA, cAMP activates another intracellular receptor, known as exchange protein directly activated by cAMP (Epac). There are two isoforms of Epac, Epac1 and Epac2, both consisting of a regulatory region that binds cAMP and a catalytic region that promotes the exchange of GDP for GTP on the small G proteins, Rap1 and Rap2 of the Ras family. In addition, Epac proteins exert their functions through interactions with a number of other cellular partners at specific cellular loci. Pathophysiological changes in Epac signalling have been associated with a wide range of diseases (Breckler, M. et al., Cell. Signal. 23: 1257-1266, 2011).

Relevant disorders that are dependent upon activation of Epac proteins by cAMP may be identified by their response to Epac inhibitors, such as ESI-09, a novel non-cyclic nucleotide Epac1 and Epac2 antagonist that is capable of specifically blocking intracellular Epac-mediated Rap1 activation and Akt phosphorylation, as well as Epac-mediated insulin secretion in pancreatic beta cells (Almahariq, M. et al., Mol. Pharmacol. 83: 122-128, 2013).

Melanoma

Epac1 has been implicated in promoting migration and metastasis in melanoma (Baljinnyam, E. et al., Pigment Cell Melanoma Res. 24: 680-687, 2011 and references cited therein). PDE4 long form activators described herein are therefore expected to be of utility in the treatment, prevention or partial control of melanoma.

Pancreatic Cancer

It has recently been shown that Epac1 is markedly elevated in human pancreatic cancer cells as compared with normal pancreas or surrounding tissue (Lorenz, R. et al., Pancreas 37: 102-103, 2008).

Pancreatic cancer is often resistant to treatments that are usually effective for other types of cancer. Using the Epac inhibitor ESI-09, a functional role of Epac1 overexpression in pancreatic cancer cell migration and invasion was demonstrated (Almahariq, M. et al., Mol. Pharmacol. 83: 122-128, 2013). These findings are consistent with results based on RNAi silencing techniques and suggest that inhibition of Epac1 signalling could be an effective therapeutic strategy for pancreatic cancer.

PDE4 long form activators described herein would therefore be expected to be of utility in the treatment, prevention or partial control of pancreatic cancer.

Diseases Dependent Upon Modulation of cAMP-Gated Ion Channels by Elevated cAMP.

In addition to activation of PKA and Epac, another effector pathway for elevated cAMP is the activation of cAMP-gated ion channels. PDE4 long form activators described herein would therefore be expected to be of utility in the treatment of disorders where inhibitors of cAMP-gated ion channels show evidence of therapeutic effects.

Diseases Associated with Increased Activity of cAMP Response Element Binding Protein.

The cAMP response element binding protein (CREB) is an important transcription factor involved in the regulation of a variety of cellular functions such as cell proliferation, differentiation, survival, and apoptosis (Cho et al., Crit Rev Oncog, 16: 37-46, 2011). CREB activity is regulated by kinase dependant phosphorylation through a range of extracellular signals, such as stress, growth factors and neurotransmitters. Phosphorylation leads to dimerisation of CREB, and together with other co-activator partner proteins, enables it to bind to promoter regions of target genes containing the cAMP response element (CRE sites) and initiate transcriptional activity. The cAMP pathway (e.g. through cAMP-dependant protein kinase mediated phosphorylation) is an important positive modulator of CREB mediated biological activity. PDE4 long form activators described herein are therefore expected to be of utility in the treatment, prevention or partial control of disorders associated with elevated CREB activity.

Leukaemia

Bone marrow cells from acute lymphoid and myeloid leukaemia patients have been reported to overexpress CREB protein and mRNA (Crans-Vargas et al., Blood, 99: 2617-9, 2002; Cho et al., Crit Rev Oncog, 16: 37-46, 2011). Furthermore, the increased CREB level correlates with poor clinical response in subjects with acute myeloid leukaemia (Crans-Vargas et al., Blood, 99: 2617-9, 2002; Shankar et al., Cancer Cell, 7:351-62, 2005). Upregulation of CREB is associated with stimulation of human leukaemia cell growth whilst downregulation inhibits myeloid cell proliferation and survival. PDE4 long form activators described herein would be expected to reduce CREB activity and function through attenuation of cAMP mediated stimulation of CREB and therefore expected to have utility in the treatment, prevention or partial control of acute lymphoid and myeloid leukaemia.

Prostate Cancer

Abnormal excessive androgen activity is an important driver in the development of prostate cancer as it stimulates the development of intraepithelial neoplasias (Merkle et al., Cellular Signalling, 23: 507-515, 2011). This is strongly supported by the use of androgen ablation approaches, involving chemical or surgical castration, in the treatment of prostate cancer. Cyclic AMP elevating agents such as forskolin can enhance androgen receptor activity through multiple intracellular mechanisms including androgen receptor activation through phosphorylation and/or interaction with CREB. Epac1 activation has also been implicated in promoting cellular proliferation in prostate cancer (Misra, U. K. and Pizzo, S. V. J. Cell. Biochem. 108: 998-1011, 2009; Misra, U. K. and Pizzo, S. V. J. Cell. Biochem. 113: 1488-1500, 2012). PDE4 long form activators described herein are therefore expected to have utility in the treatment, prevention or partial control of prostate cancer.

Diseases Associated with Reduced Activity of cAMP-Hydrolysing PDE Enzymes

Loss-of-function mutations in gene(s) for cAMP-hydrolysing PDE isoforms other than PDE4, such as PDE8 and PDE11, have been detected in a number of diseases (Vezzosi, D. and Bertherat, J., Eur. J. Endocrinol. 165: 177-188, 2011; Levy, I. et al., Curr. Opin. Pharmacol. 11: 689-697, 2011; Azevedo, M. F. and Stratakis, C. A. Endocr. Pract. 17 Suppl 3: 2-7, 2011). These mutations can lead to abnormally high cAMP levels and/or duration of cAMP action with pathological consequences as detailed below. PDE4 long form activators described herein are therefore expected to be of utility in the treatment, prevention or partial control of these diseases, such as adrenocortical tumours, testicular cancer, PPNAD and Carney Complex.

Adrenocortical Tumours

Adrenocortical tumours associated with an inactivating point mutation in the gene encoding PDE11A4 have decreased expression of PDE11A4 and increased cAMP levels (Horvath, A. et al., Nat Genet. 38: 794-800, 2006; Horvath, A. et al., Cancer Res. 66: 11571-11575, 2006; Libe, R., et al., Clin. Cancer Res. 14: 4016-4024, 2008).

Testicular Cancer

Mutations that reduce PDE11A activity and increase cAMP levels have been observed in some forms of testicular cancer (Horvath. A. et al., Cancer Res. 69: 5301-5306, 2009).

Primary Pigmented Nodular Adrenocortical Diseases (PPNAD)

Mutations in the PDE8B gene have also been identified as a predisposing factor for PPNAD and the mutant protein shows reduced ability to degrade cAMP (Horvath, A., Mericq, V. and Stratakis, C. A. N. Engl. J. Med. 358: 750-752, 2008; Horvath, A. et al., Eur. J. Hum. Genet. 16: 1245-1253, 2008).

Carney Complex

In Carney Complex (CNC) caused by PRKAR1A mutations, some patients also have defects in PDE11A that may exert a synergistic effect to enhance abnormal activation of the cAMP signal transduction pathway, leading to adrenal and testicular cancer (Libé, R. et al., J. Clin. Endocrinol. Metab. 96: E208-214, 2011).

Treatment and Posoloqy

By “treatment” herein is meant the treatment by therapy, whether of a human or a non-human animal (e.g., in veterinary applications) typically a non-human mammal, in which some desired therapeutic effect on the condition is achieved; for example, the inhibition of the progress of the disorder, including a reduction in the rate of progress, a halt in the rate of progress, amelioration of the disorder or cure of the condition. Treatment as a prophylactic measure is also included. References herein to prevention or prophylaxis do not indicate or require complete prevention of a condition; its manifestation may instead be reduced or delayed via prophylaxis or prevention according to the present invention.

Compounds or compositions as dercibed herein, when used for preventing or treating a disorder, may be administered in an “effective amount”, which may also be referred to as a “therapeutically effective amount”. By a “therapeutically effective amount” herein is meant an amount of the one or more compounds described herein or a pharmaceutical formulation comprising such one or more compounds, which is effective for producing such a therapeutic effect, commensurate with a reasonable benefit/risk ratio.

It will be appreciated that appropriate dosages of the compounds described herein may vary from patient to patient. Determining the optimal dosage will generally involve the balancing of the level of therapeutic benefit against any risk or deleterious side effects of the treatments of the present invention. The selected dosage level will depend on a variety of factors including the activity of the particular compound, the route of administration, the time of administration, the rate of excretion of the compound, the duration of the treatment, other drugs, compounds or materials used in combination and the age, sex, weight, condition, general health and prior medical history of the patient. The amount of compound(s) and route of administration will ultimately be at the discretion of the physician, although generally the dosage will be to achieve local concentrations at the site of action so as to achieve the desired effect. Administration in vivo can be effected in one dose, continuously or intermittently throughout the course of treatment. Methods of determining the most effective means and dosage of administration are well known to a person skilled in the art and will vary with the formulation used for therapy, the purpose of the therapy, the target cell being treated, and the subject being treated. Single or multiple administrations can be carried out with the dose level and pattern being selected by the treating physician.

In general, a suitable dose of the one or more compounds described herein may be in the range of about 0.001 to 50 mg/kg body weight of the subject per day, preferably in a dosage of 0.01-25 mg per kg body weight per day, e.g., 0.01, 0.05, 0.10, 0.25, 0.50, 1.0, 2.5, 10 or 25 mg/kg per day. Where the compound(s) is a salt, solvate, prodrug or the like, the amount administered may be calculated on the basis of the parent compound and so the actual weight to be used may be increased proportionately.

Combination Therapies

The compounds described herein may also find application in mimicking or enhancing the effects of drugs known to produce their therapeutic effect through lowering of intracellular cAMP levels.

A number of therapeutically beneficial drugs have a primary mode of action involving lowering intracellular cAMP levels and/or cAMP-mediated activity, as summarised below. Since PDE4 long form activators described herein will also act to lower cAMP levels it is expected that these agents will mimic and/or augment the pharmacological properties and therapeutic utility of drugs operating through a down-regulation of cAMP-mediated signalling. In certain embodiments, a compound described herein is therefore provided as part of a combination therapy with another agent that lowers intracellular cAMP levels and/or cAMP-mediated activity. The combination therapy may be administered simultaneously, contemporaneously, sequentially or separately. In one embodiment, the compound described herein and the separate cAMP lowering agent are provided in a single composition, as described in more detail below. The combination therapy may comprise a described herein and one or more of:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.

Combination with α-2 Adrenergic Receptor Agonist

α-2 Adrenergic receptor stimulation is known to reduce cAMP levels through a Gi protein-mediated inhibition of adenylyl cyclase activity in a broad range of tissues. In noradrenergic neurones in the brain and peripheral sympathetic nervous system, presynaptic α-2 adrenergic receptor activation inhibits noradrenaline release and noradrenergic activity. Drugs (e.g. clonidine, dexmedetomidine, guanfacine) that act as agonists at these receptors are effective in the treatment of a variety of clinical conditions. Clonidine, the prototypic agent, has shown therapeutic utility in the treatment of hypertension, neuropathic pain, opioid detoxification, insomnia, ADHD, Tourette syndrome, sleep hyperhidrosis, addiction (narcotic, alcohol and nicotine withdrawal symptoms), migraine, hyperarousal, anxiety and also as a veterinary anaesthetic. Lowering of cAMP levels by PDE4 long form activation may be expected to yield similar effects to drugs acting through α-2 adrenergic receptor stimulation. Furthermore, PDE4 long form activators described herein may be expected to potentiate the pharmacodynamic effects of α-2 adrenergic receptor agonists when used in combination.

Combination with δ-1 Adrenergic Receptor Antagonist

β-1 Adrenergic receptor antagonists are used in the treatment a range of cardiovascular indications including hypertension, cardiac arrhythmias and cardioprotection following myocardial infarction. Their primary mechanism of action involves reducing the effects of excessive circulating adrenaline and sympathetic activity, mediated by noradrenaline, particularly at cardiac β-1 adrenergic receptors. Endogenous and synthetic β-1 adrenergic receptor agonists stimulate adenylyl cyclase activity through Gs activation and raise intracellular cAMP levels in a variety of tissues such as heart and kidney. Consequently, drugs that block β-1 adrenergic receptor mediated activity exert their pharmacological effects by attenuating the increase in cAMP mediated signalling. Given that PDE4 long form activation will also lower cAMP concentration and transduction in cardiac tissue, PDE4 long form activators described herein may be expected to find utility in the treatment or partial control of hypertension, cardiac arrhythmias, congestive heart failure and cardioprotection. Additional non-cardiovascular therapeutic utility may be expected in disorders such as post-traumatic stress related disorder, anxiety, essential tremor and glaucoma, which also respond to β-1 adrenergic antagonist treatment. Furthermore, PDE4 long form activators described herein may be expected to potentiate the pharmacodynamic effects of β-1 adrenergic receptor antagonists when used in combination.

Methods of Treatment

Compounds as described here may be used for treating or preventing a disease or disorder that can be ameliorated by activation of long isoforms of PDE4. Compounds as described herein may be used for treating or preventing a disease or disorder mediated by excessive intracellular cyclic AMP signalling. In a further aspect, the present invention provides a small molecule activator of a PDE4 long form described herein for use in a method for the treatment or prevention of a disease or disorder in a patient in need of such therapy. The invention also provides a method of treating or preventing a disease or disorder in a patient in need thereof, comprising administering to a patient in need thereof an effective amount of a compound described herein. The invention provides a method of treating or preventing a disease or disorder that can be ameliorated by activation of long isoforms of PDE4, comprising administering to a patient in need thereof a therapeutically effective amount of any compound or a pharmaceutically acceptable salt or derivative as described herein. The invention provides a method of treating or preventing a disease or disorder mediated by excessive intracellular cyclic AMP signalling, comprising administering to a patient in need thereof a therapeutically effective amount of any compound or a pharmaceutically acceptable salt or derivative as described herein. The disease or disorder may be any disease of disorder described herein, including: a disease associated with increased cAMP production and signalling (such as hyperthyroidism, Jansens's metaphyseal chondrodysplasia, hyperparathyroidism, familial male-limited precocious puberty, pituitary adenomas, Cushing's disease, polycystic kidney disease, polycystic liver disease, MODY5 and cardiac hypertrophy); diseases known to be associated with increased cAMP-mediated signalling, including disorders associated with activating mutations of the alpha subunit of the G protein (GNAS1) (such as McCune-Albright syndrome); amelioration of toxin-induced increases in adenylyl cyclase activity in infectious diseases (such as cholera, whooping cough, anthrax, and tuberculosis); treatment of diseases known to be dependent upon activation of PKA by elevated cAMP (such as HIV infection and AIDS, and Common Variable Immunodeficiency (CVID)); treatment of diseases known to be dependent upon activation of either or both of Epac1 and Epac2 by elevated cAMP (such as melanoma and pancreatic cancer); treatment of diseases dependent upon modulation of cAMP-gated ion channels by elevated cAMP; treatment of diseases known to be associated with increased activity of cAMP response element binding protein (such as leukaemia and prostate cancer); treatment of diseases known to be associated with reduced activity of cAMP-hydrolysing PDE enzymes (such as adrenocortical tumours, testicular cancer, primary pigmented nodular adrenocortical diseases (PPNAD) and Carney Complex); and mimicking or enhancing the effects of drugs known to produce their therapeutic effect through lowering of intracellular cAMP levels.

As used herein, the terms “compound of the invention”, “compound of the disclosure” “compound described herein” and “compound of Formula I”, etc, include pharmaceutically acceptable salts and derivatives thereof and polymorphs, isomers (e.g. stereoisomers and tautomers) and isotopically labelled variants thereof. For example, reference to compounds of Formula I includes pharmaceutically acceptable salts thereof. Furthermore, these terms include all the sub-embodiments of those compounds disclosed herein, including compunds of Formula A to D, I to IV and Z, and all embodiments thereof.

A compound described herein may be provided as a solvate, for example a hydrate.

Pharmaceutically acceptable derivatives of a compound described herein include pharmaceutically acceptable esters, amides, prodrugs (e.g. a pyridine N-oxide) or isotopically labelled variants thereof. The present invention further provides pharmaceutical compositions comprising a compound described herein, including a pharmaceutically acceptable salt, solvate, ester, hydrate or amide thereof, in admixture with a pharmaceutically acceptable excipient(s), and optionally other therapeutic agents. The term “acceptable” means being compatible with the other ingredients of the composition and not deleterious to the recipient thereof. Compositions include e.g. those suitable for oral, sublingual, subcutaneous, intravenous, epidural, intrathecal, intramuscular, transdermal, intranasal, pulmonary, topical, local, or rectal administration, and the like, typically in unit dosage forms for administration.

The term “pharmaceutically acceptable salt” includes a salt prepared from pharmaceutically acceptable non-toxic acids or bases including inorganic or organic acids and bases. Compounds which contain basic, e.g. amino, groups are capable of forming pharmaceutically acceptable salts with acids. Examples of pharmaceutically acceptable acid addition salts of the compounds described herein include acid addition salts formed with organic carboxylic acids such as acetic, lactic, tartaric, maleic, citric, pyruvic, oxalic, fumaric, oxaloacetic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids.

Compounds which contain acidic, e.g. carboxyl, groups are capable of forming pharmaceutically acceptable salts with bases. Pharmaceutically acceptable basic salts of the compounds described herein include, but are not limited to, metal salts such as alkali metal or alkaline earth metal salts (e.g. sodium, potassium, magnesium or calcium salts) and zinc or aluminium salts and salts formed with ammonia or pharmaceutically acceptable organic amines or heterocyclic bases such as ethanolamines (e.g. diethanolamine), benzylamines, N-methyl-glucamine, amino acids (e.g. lysine) or pyridine.

Hemisalts of acids and bases may also be formed, e.g. hemisulphate salts.

Pharmaceutically acceptable salts of compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002).

Prodrugs

Compounds described herein may be provided as a prodrug. Prodrugs are derivatives of compounds of Formula I (which may have little or no pharmacological activity themselves), which can, when administered in vivo, be converted into compounds of Formula I.

Prodrugs can, for example, be produced by replacing functionalities present in the compounds of Formula I with appropriate moieties which are metabolised in vivo to form a compound of Formula I. The design of prodrugs is well-known in the art, as discussed in Bundgaard, Design of Prodrugs 1985 (Elsevier), The Practice of Medicinal Chemistry 2003, 2^{nd} Ed, 561-585 and Leinweber, Drug Metab. Res. 1987, 18: 379.

In vivo metabolism of prodrugs of compounds of Formula I may for example involve hydrolysis, oxidative metabolism or reductive metabolism of the prodrug. Examples of prodrugs of compounds of Formula I are amides and esters of those compounds that may be hydrolysed in vivo. For example, where the compound of Formula I contains a carboxylic acid group (—COOH), the hydrogen atom of the carboxylic acid group may be replaced in order to form an ester (e.g. the hydrogen atom may be replaced by C₁₋₆alkyl). Where a compound contains an alcohol group (—OH), the hydrogen atom of the alcohol group may be replaced in order to form an ester (e.g. the hydrogen atom may be replaced by —C(O)C₁₋₆alkyl). Further examples of prodrugs of compounds of Formula I include pyridine N-oxides that may be reductively metabolised in vivo to form compounds of Formula I containing a pyridine ring.

Solvates

It may be convenient or desirable to prepare, purify, and/or handle a corresponding solvate of the compounds described herein, which may be used in the any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate.

Isomers

It will be appreciated that the compounds described herein may exist in various isomeric forms and the compounds described herein include all stereoisomeric forms and mixtures thereof, including enantiomers and racemic mixtures. The present invention includes within its scope the use of any such stereoisomeric form or mixture of stereoisomers, including the individual enantiomers of the compounds of Formula I as well as wholly or partially racemic mixtures of such enantiomers. Where appropriate, isomers can be separated from their mixtures by the application or adaptation of known methods (e.g. chromatographic techniques and recrystallisation techniques). Where appropriate, isomers can be prepared by the application or adaptation of known methods (e.g. asymmetric synthesis). In addition, it will be appreciated that in some instances, compounds described herein may exist in tautomeric forms and the compounds described herein include all tautomers and mixtures thereof.

Isotopes The compounds described herein invention includes pharmaceutically acceptable isotopically-labelled compounds wherein one or more atoms are replaced by atoms having the same atomic number, but an atomic mass or mass number different from the atomic mass or mass number usually found in nature.

Examples of isotopes suitable for inclusion in the compounds described herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, and sulphur, such as ³⁵S. Certain isotopically-labelled compounds, for example, those incorporating a radioactive isotope, are useful in drug and/or substrate tissue distribution studies. The radioactive isotopes ³H and ¹⁴C are particularly useful for this purpose in view of their ease of incorporation and ready means of detection. Substitution with positron emitting isotopes, such as ¹¹C, ¹⁸F, ¹⁵O and ¹³N, can be useful in Positron Emission Topography (PET) studies for examining substrate receptor occupancy. It is well known in the art that isotope substitution of a hydrogen atom that is bonded to carbon with deuterium [²H] may positively influence the ADME properties of drug candidates by slowing CYP-mediated metabolism [for a review see Nat. Rev. Drug Discov. 15(4): 219-21 (2016)].

Isotopically-labelled compounds can generally be prepared by conventional techniques known to those skilled in the art or by processes analogous to those described herein using an appropriate isotopically-labelled reagent in place of the non-labelled reagent previously employed.

Pharmaceutical Compositions

A pharmaceutical composition may comprise any compound or a pharmaceutically acceptable salt or derivative as described herein, and a pharmaceutically acceptable excipient. A pharmaceutical composition as described herein may comprise one or more pharmaceutically acceptable excipients, for example pharmaceutically acceptable carriers, diluents, preserving agents, solubilising agents, stabilising agents, disintegrating agents, binding agents, lubricating agents, wetting agents, emulsifiers, sweeteners, colourants, odourants, salts, buffers, coating agents and antioxidants. Suitable excipients and techniques for formulating pharmaceutical compositions are well known in the art (see, e.g. Remington: The Science and Practice of Pharmacy, 20th Ed., ed. A. Gennaro, Lippincott Williams & Wilkins, 2000). Suitable excipients include, without limitation, pharmaceutical grade starch, mannitol, lactose, corn starch, magnesium stearate, stearic acid, alginic acid, sodium saccharin, talcum, cellulose, cellulose derivatives (e.g. hydroxypropylmethylcellulose, carboxymethylcellulose) glucose, sucrose (or other sugar), sodium carbonate, calcium carbonate, magnesium carbonate, sodium phosphate, calcium phosphate, gelatin, agar, pectin, liquid paraffin oil, olive oil, alcohol, detergents, emulsifiers or water (preferably sterile).

A pharmaceutical composition may further comprise an adjuvant and/or one or more additional therapeutically active agent(s).

A pharmaceutical composition may be provided in unit dosage form, will generally be provided in a sealed container and may be provided as part of a kit. Such a kit would normally (although not necessarily) include instructions for use. It may include a plurality of said unit dosage forms.

A pharmaceutical composition may be adapted for administration by any appropriate route, for example by oral, buccal or sublingual routes or parenteral routes, including subcutaneous, intramuscular, intravenous, intraperitoneal, and intradermal, rectal and topical administration, and inhalation. Such compositions may be prepared by any method known in the art of pharmacy, for example by admixing the active ingredient with a excipient(s) under sterile conditions.

For oral administration, the active ingredient may be presented as discrete units, such as tablets, capsules, powders, granulates, solutions, suspensions, and the like.

Formulations suitable for oral administration may also be designed to deliver the compounds described herein in an immediate release manner or in a rate-sustaining manner, wherein the release profile can be delayed, pulsed, controlled, sustained, or delayed and sustained or modified in such a manner which optimises the therapeutic efficacy of the said compounds. Means to deliver compounds in a rate-sustaining manner are known in the art and include slow release polymers that can be formulated with the said compounds to control their release.

Examples of rate-sustaining polymers include degradable and non-degradable polymers that can be used to release the said compounds by diffusion or a combination of diffusion and polymer erosion. Examples of rate-sustaining polymers include hydroxypropyl methylcellulose, hydroxypropyl cellulose, methyl cellulose, ethyl cellulose, sodium carboxymethyl cellulose, polyvinyl alcohol, polyvinyl pyrrolidone, xanthum gum, polymethacrylates, polyethylene oxide and polyethylene glycol.

Liquid (including multiple phases and dispersed systems) formulations include emulsions, suspensions, solutions, syrups and elixirs. Such formulations may be presented as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds described herein may also be used in fast-dissolving, fast-disintegrating dosage forms such as those described in Liang and Chen, Expert Opinion in Therapeutic Patents 2001, 11(6): 981-986.

The formulation of tablets is discussed in H. Lieberman and L. Lachman, Pharmaceutical Dosage Forms: Tablets 1980, vol. 1 (Marcel Dekker, New York).

For administration intranasally or by inhalation, the active ingredient may be presented in the form of a dry powder from a dry powder inhaler or in the form of an aerosol spray of a solution or suspension from a pressurised container, pump, spray, atomiser or nebuliser.

For parenteral administration, the pharmaceutical composition of the invention may be presented in unit-dose or multi-dose containers, e.g. injection liquids in predetermined amounts, for example in sealed vials and ampoules, and may also be stored in a freeze dried (lyophilized) condition requiring only the addition of sterile liquid carrier, e.g. water, prior to use.

For parenteral administration, the compounds described herein may be administered directly into the blood stream, into subcutaneous tissue, into muscle, or into an internal organ. Suitable means for administration include intravenous, intraarterial, intrathecal, intraventricular, intraurethral, intrasternal, intracranial, intramuscular, intrasynovial and subcutaneous. Suitable devices for administration include needle (including microneedle) injectors, needle-free injectors and infusion techniques.

Parenteral formulations are typically aqueous or oily solutions. Where the solution is aqueous, excipients such as sugars (including but not restricted to glucose, mannitol, sorbitol, etc.) salts, carbohydrates and buffering agents (preferably to a pH of from 3 to 9) may be used. For some applications, the compounds described herein may be more suitably formulated as a sterile non-aqueous solution or as a dried form to be used in conjunction with a suitable vehicle such as sterile, pyrogen-free water (WFI).

Parenteral formulations may include implants derived from degradable polymers such as polyesters (e.g. polylactic acid, polylactide, polylactide-co-glycolide, polycapro-lactone, polyhydroxybutyrate), polyorthoesters and polyanhydrides. These formulations may be administered via surgical incision into the subcutaneous tissue, muscular tissue or directly into specific organs.

The preparation of parenteral formulations under sterile conditions, for example, by lyophilisation, may readily be accomplished using standard pharmaceutical techniques well known to those skilled in the art.

The solubility of compounds described herein used in the preparation of parenteral solutions may be increased by the use of appropriate formulation techniques, such as the incorporation of co-solvents and/or solubility-enhancing agents such as surfactants, micelle structures and cyclodextrins.

Mixed with such pharmaceutically acceptable excipients, e.g. as described in the standard reference, Gennaro, A. R. et al, Remington: The Science and Practice of Pharmacy (21st Edition, Lippincott Williams & Wilkins, 2005, see especially Part 5: Pharmaceutical Manufacturing), the active agent may be compressed into solid dosage units, such as pills, tablets, or be processed into capsules, suppositories or patches. By means of pharmaceutically acceptable liquids the active agent can be applied as a fluid composition, e.g. as an injection preparation or as an aerosol spray, in the form of a solution, suspension, or emulsion.

For making solid dosage units, the use of conventional additives such as fillers, colorants, polymeric binders and the like is contemplated. In general any pharmaceutically acceptable additive that does not interfere with the function of the active compounds can be used. Suitable carriers with which the active agent described herein can be administered as solid compositions include lactose, starch, cellulose derivatives and the like, or mixtures thereof, used in suitable amounts. For parenteral administration, aqueous suspensions, isotonic saline solutions and sterile injectable solutions may be used, containing pharmaceutically acceptable dispersing agents and/or wetting agents, such as propylene glycol or butylene glycol.

The invention further includes a pharmaceutical composition, as hereinbefore described, in combination with packaging material suitable for said composition, said packaging material including instructions for the use of the composition for the use as hereinbefore described.

In some embodiments, the one or more compounds described herein may be used in combination therapies for the treatment of the described conditions i.e., in conjunction with other therapeutic agents. For the case of active compounds combined with other therapies the two or more treatments may be given in individually varying dose schedules and via different routes.

The combination of the agents listed above with a compound described herein would be at the discretion of the physician who would select dosages using his common general knowledge and dosing regimens known to a skilled practitioner.

Where a compound described herein is administered in combination therapy with one, two, three, four or more, preferably one or two, preferably one other therapeutic agents, the compounds can be administered simultaneously or sequentially. When administered sequentially, they can be administered at closely spaced intervals (for example over a period of 5-10 minutes) or at longer intervals (for example 1, 2, 3, 4 or more hours apart, or even longer period apart where required), the precise dosage regimen being commensurate with the properties of the therapeutic agent(s).

In one embodiment, the invention provides a product comprising a compound described herein and another therapeutic agent as a combined preparation for simultaneous, separate or sequential use in therapy. In one embodiment, the therapy is the treatment or prevention of disorders where a reduction of second messenger responses mediated by cyclic 3′,5′-adenosine monophosphate (cAMP) is required. Products provided as a combined preparation include a composition comprising a compound described herein and the other therapeutic agent together in the same pharmaceutical composition, or the compound described herein and the other therapeutic agent in separate form, e.g. in the form of a kit.

In one embodiment, the invention provides a pharmaceutical composition comprising a compound of the invention and another therapeutic agent. Optionally, the pharmaceutical composition may comprise a pharmaceutically acceptable excipient, as described above.

In one embodiment, the invention provides a kit comprising two or more separate pharmaceutical compositions, at least one of which contains a compound described herein. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound described herein and the other therapeutic agent may be manufactured and/or formulated by the same or different manufacturers. Moreover, the compound described herein and the other therapeutic may be brought together into a combination therapy: (i) prior to release of the combination product to physicians (e.g. in the case of a kit comprising the compound described herein and the other therapeutic agent); (ii) by the physician themselves (or under the guidance of the physician) shortly before administration; (iii) in the patient themselves, e.g. during sequential administration of the compound described herein and the other therapeutic agent.

Method of Manufacture & Method of Treatment

The invention also provides the use of a compound described herein in the manufacture of a medicament for the treatment or prevention of disorders where a reduction of second messenger responses mediated by cyclic 3′,5′-adenosine monophosphate (cAMP) is required, wherein the medicament is prepared for administration with another therapeutic agent. The invention also provides the use of another therapeutic agent in the manufacture of medicament for treating a disease or condition mediated by cAMP for the treatment or prevention of disorders where a reduction of second messenger responses mediated by cAMP is required, wherein the medicament is prepared for administration with a compound described herein.

The invention also provides a compound described herein for use in the treatment or prevention of disorders where a reduction of second messenger responses mediated by cAMP is required, wherein the compound described herein is prepared for administration with another therapeutic agent. The invention also provides another therapeutic agent for use in the treatment or prevention of disorders where a reduction of second messenger responses mediated by cAMP is required, wherein the other therapeutic agent is prepared for administration with a compound described herein. The invention also provides a compound described herein for use in for the treatment or prevention of disorders where a reduction of second messenger responses mediated by cAMP is required, wherein the compound described herein is administered with another therapeutic agent. The invention also provides another therapeutic agent for use in the treatment or prevention of disorders where a reduction of second messenger responses mediated by cAMP is required, wherein the other therapeutic agent is administered with a compound described herein.

The invention also provides the use of a compound described herein in the manufacture of a medicament for the treatment or prevention of disorders where a reduction of second messenger responses mediated by cAMP is required, wherein the patient has previously (e.g. within 24 hours) been treated with another therapeutic agent. The invention also provides the use of another therapeutic agent in the manufacture of a medicament for the treatment or prevention of disorders where a reduction of second messenger responses mediated by cAMP is required, wherein the patient has previously (e.g. within 24 hours) been treated with a compound described herein.

In one embodiment, the other therapeutic agent is:
(i) a presynaptic α-2 adrenergic receptor agonist, optionally clonidine, dexmedetomidine, or guanfacine;
(ii) a β-1 Adrenergic receptor antagonist (“beta-blocker”), optionally Atenolol, Metoprolol, Bisoprolol, Acebutolol, or Betaxolol.

### EXAMPLES

The present invention will now be further described by way of the following non-limiting examples and with reference to the Tables and Figures: Table 1 shows the structures of small molecule PDE4 long form activators, Examples 1 to 292, according to the present invention.

Table 2 shows enzyme assay data for PDE4D5, a long form of PDE4.

Table 3 shows enzyme assay data for PDE4C3, another long form of PDE4.

Table 4 shows enzyme assay data for PDE4B2, a short form of PDE4.

Table 5 shows inhibition of PGE2-stimulated cyst formation in a 3D culture of m-IMCD3 kidney cells treated with compounds of the present invention.

Table 6 shows inhibition of PGE2-stimulated cyst formation in a 3D culture of MDCK kidney cells treated with compounds of the present invention.

Table 7 shows reduction of cAMP levels in m-IMCD3 kidney cell culture treated with compounds of the present invention

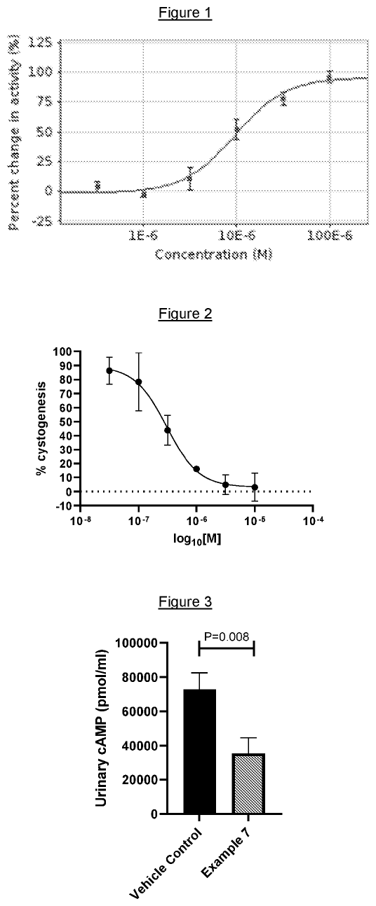

FIG. 1 shows concentration-dependent activation of a PDE4 long form, PDE4D5, by Example 66.

FIG. 2 shows concentration-dependent inhibition of PGE2-stimulated cyst formation in a 3D culture of m-IMCD3 cells treated with Example 191.

FIG. 3 shows inhibition by Example 7 of PTH-induced cAMP elevation in rat urine.

Experimental Details

### Preparation of Examples 1 to 292

Reactions were monitored by thin layer chromatography (Merck Millipore TLC Silica Gel 60 F₂₅₄). Flash column chromatography was performed on Biotage Isolera® using pre-packed silica gel columns. NMR spectra were recorded using a Bruker 300 or 400 MHz spectrometers, using residual signal of deuterated solvent as internal reference at 25° C. Exchangeable NH and OH residues were not identifiable in the ¹H NMR spectra in some cases. The following abbreviations are used in the experimental details: CDI (1,1′-carbonyldiimidazole), DCM (dichloromethane), DIPEA (N,N-diisopropylethylamine), DMF (dimethylformamide), EDC (N-ethyl-N′-(3-dimethylaminopropyl)carbodiimide), h (hours), HOBt (hydroxybenzotriazole), r.t. (room temperature), SEM [2-(trimethylsilyl)ethoxymethyl], SFC (supercritical fluid chromatography), TBDPS (tert-butyldiphenylsilyl), THF (tetrahydrofuran). The following abbreviations are used in the assignment of NMR signals: s (singlet), d (doublet), t (triplet), q (quartet), m (multiplet), app. (approximate), br. (broad), dd (double doublet), dt (double triplet), td (triple doublet).

### General Procedure 1: Synthesis of chiral amines

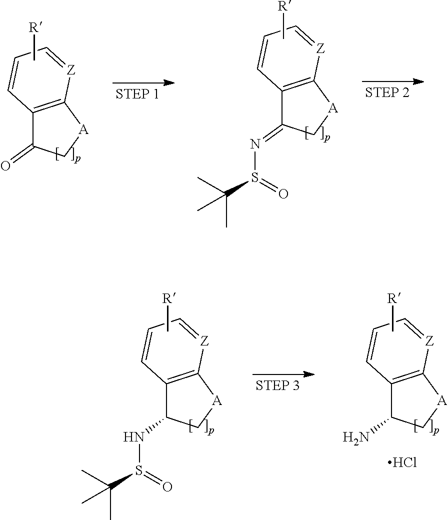

(In the above scheme, when utilised to synthesise a compound of Formula I, it will be appreciated that Z may be C or N, A may be C or a heteroatom (e.g. O), p is 1 or 2 and R′ is absent or represents one or more substituents suitable to provide a compound of Formula I.)

Step 1:

To a stirred solution of ketone (1.0 equiv.) and (S)-2-methylpropane-2-sulfinamide (3.0 equiv.) in dry THF (0.25 M in substrate) was added titanium(IV) ethoxide (5 equiv.). The resulting mixture was stirred at 70° C. for 16 h. The reaction mixture was cooled to ambient temperature and then diluted with brine and EtOAc. The resulting suspension was filtered through Celite® and the filter cake was washed with EtOAc. The organic layer from the filtrate was separated, dried (Na₂SO₄) and concentrated under reduced pressure. The crude product was purified by flash column chromatography to afford the desired product:
(S)-N-(chroman-4-ylidene)-2-methylpropane-2-sulfinamide;
(S)-N-(8-fluorochroman-4-ylidene)-2-methylpropane-2-sulfinamide;
(S)-N-(7-fluorochroman-4-ylidene)-2-methylpropane-2-sulfinamide;
(S)-N-(6-fluorochroman-4-ylidene)-2-methylpropane-2-sulfinamide;
(S)-N-(5-fluorochroman-4-ylidene)-2-methylpropane-2-sulfinamide;
(S)-N-(7-methoxychroman-4-ylidene)-2-methylpropane-2-sulfinamide;
(S)-N-(6-methoxychroman-4-ylidene)-2-methylpropane-2-sulfinamide;
(S)-N-(6-chlorochroman-4-ylidene)-2-methylpropane-2-sulfinamide;
(S)-N-(7-cyanochroman-4-ylidene)-2-methylpropane-2-sulfinamide;
(S)-N-(7-fluoro-2,3-dihydro-1H-inden-1-ylidene)-2-methylpropane-2-sulfinamide;
(S)-N-(6-fluoro-2,3-dihydro-1H-inden-1-ylidene)-2-methylpropane-2-sulfinamide;
(S)-N-(5-fluoro-2,3-dihydro-1H-inden-1-ylidene)-2-methylpropane-2-sulfinamide;
(S)-N-(4-fluoro-2,3-dihydro-1H-inden-1-ylidene)-2-methylpropane-2-sulfinamide;
(S)-N-(7-methoxy-2,3-dihydro-1H-inden-1-ylidene)-2-methylpropane-2-sulfinamide;
(S)-N-(6-methoxy-2,3-dihydro-1H-inden-1-ylidene)-2-methylpropane-2-sulfinamide;
(S)-N-(5-methoxy-2,3-dihydro-1H-inden-1-ylidene)-2-methylpropane-2-sulfinamide;
(S)-N-(4-methoxy-2,3-dihydro-1H-inden-1-ylidene)-2-methylpropane-2-sulfinamide;
(S)-N-(6-cyano-2,3-dihydro-1H-inden-1-ylidene)-2-methylpropane-2-sulfinamide;
(S)-N-(5-cyano-2,3-dihydro-1H-inden-1-ylidene)-2-methylpropane-2-sulfinamide;
(S)-N-(4-cyano-2,3-dihydro-1H-inden-1-ylidene)-2-methylpropane-2-sulfinamide;
(S)-N-(6,7-dihydro-5H-cyclopenta[b]pyridin-5-ylidene)-2-methylpropane-2-sulfinamide.

Similarly, the corresponding (R)-configured sulfinamides may be made using (R)-2-methylpropane-2-sulfinamide instead of (S)-2-methylpropane-2-sulfinamide.

Step 2:

The N-sulfinyl imine (1.0 equiv.) was dissolved in wet THF (2-3% water; 0.31 M in substrate) and cooled to 0° C. Sodium borohydride (3.0 equiv.) was added in a single portion. The mixture was then stirred for 30 min at 0° C., after which time the bath temperature was allowed to gradually rise to ambient temperature. The reaction mixture was stirred at ambient temperature for 16 h (monitored by TLC). The mixture was then concentrated under reduced pressure to remove THF and diluted with DCM. The mixture was washed with water followed by brine, dried (Na₂SO₄) and concentrated under reduced pressure. The crude product was processed by flash column chromatography or/and SFC to separate the mixture of diastereoisomers and afford the required major diastereoisomer with good purity:
(S)-N-[(S)-6-chroman-4-yl]-2-methylpropane-2-sulfinamide;
(S)-N-[(S)-8-fluorochroman-4-yl]-2-methylpropane-2-sulfinamide;
(S)-N-[(S)-7-fluorochroman-4-yl]-2-methylpropane-2-sulfinamide;
(S)-N-[(S)-6-fluorochroman-4-yl]-2-methylpropane-2-sulfinamide;
(S)-N-[(S)-5-fluorochroman-4-yl]-2-methylpropane-2-sulfinamide;
(S)-N-[(S)-7-methoxychroman-4-yl]-2-methylpropane-2-sulfinamide;
(S)-N-[(S)-6-methoxychroman-4-yl]-2-methylpropane-2-sulfinamide;
(S)-N-[(S)-6-chlorochroman-4-yl]-2-methylpropane-2-sulfinamide;
(S)-N-[(S)-7-cyanochroman-4-yl]-2-methylpropane-2-sulfinamide;
(S)-N-[(S)-7-fluoro-2,3-dihydro-1H-inden-1-yl]-2-methylpropane-2-sulfinamide;
(S)-N-[(S)-6-fluoro-2,3-dihydro-1H-inden-1-yl]-2-methylpropane-2-sulfinamide;
(S)-N-[(S)-5-fluoro-2,3-dihydro-1H-inden-1-yl]-2-methylpropane-2-sulfinamide;
(S)-N-[(S)-4-fluoro-2,3-dihydro-1H-inden-1-yl]-2-methylpropane-2-sulfinamide;
(S)-N-[(S)-7-methoxy-2,3-dihydro-1H-inden-1-yl]-2-methylpropane-2-sulfinamide;
(S)-N-[(S)-6-methoxy-2,3-dihydro-1H-inden-1-yl]-2-methylpropane-2-sulfinamide;
(S)-N-[(S)-5-methoxy-2,3-dihydro-1H-inden-1-yl]-2-methylpropane-2-sulfinamide;
(S)-N-[(S)-4-methoxy-2,3-dihydro-1H-inden-1-yl]-2-methylpropane-2-sulfinamide;
(S)-N-[(S)-6-cyano-2,3-dihydro-1H-inden-1-yl]-2-methylpropane-2-sulfinamide;
(S)-N-[(S)-5-cyano-2,3-dihydro-1H-inden-1-yl]-2-methylpropane-2-sulfinamide;
(S)-N-[(S)-4-cyano-2,3-dihydro-1H-inden-1-yl]-2-methylpropane-2-sulfinamide;
(S)-N-[(S)-6,7-dihydro-5H-cyclopenta[b]pyridin-5-yl]-2-methylpropane-2-sulfinamide;

Similarly, the corresponding (R,R)-configured sulfinamides may be made using the enantiomeric (R)-sulfinamide starting materials.

Step 3:

To an ice-cold solution of sulfinamide (1 equiv.) in DCM (0.33 M in substrate) was added 4 N HCl in 1,4-dioxane (10 equiv. HCl). The resulting mixture was stirred at ambient temperature for 16 h (monitored by TLC). The mixture was then concentrated under reduced pressure to give a residue that was triturated with hexanes and dried to afford the amine hydrochloride salt as a solid:
(S)-chroman-4-amine. HCl;
(S)-8-fluorochroman-4-amine. HCl;
(S)-7-fluorochroman-4-amine. HCl;
(S)-6-fluorochroman-4-amine. HCl;
(S)-5-fluorochroman-4-amine. HCl;
(S)-7-methoxychroman-4-amine. HCl;
(S)-6-methoxychroman-4-amine. HCl;
(S)-6-chlorochroman-4-amine. HCl;
(S)-7-cyanochroman-4-amine. HCl;
(S)-7-fluoro-2,3-dihydro-1H-inden-1-amine. HCl;
(S)-6-fluoro-2,3-dihydro-1H-inden-1-amine. HCl;
(S)-5-fluoro-2,3-dihydro-1H-inden-1-amine. HCl;
(S)-4-fluoro-2,3-dihydro-1H-inden-1-amine. HCl;
(S)-7-methoxy-2,3-dihydro-1H-inden-1-amine. HCl;
(S)-6-methoxy-2,3-dihydro-1H-inden-1-amine. HCl;
(S)-5-methoxy-2,3-dihydro-1H-inden-1-amine. HCl;
(S)-4-methoxy-2,3-dihydro-1H-inden-1-amine. HCl;
(S)-6-cyano-2,3-dihydro-1H-inden-1-amine. HCl;
(S)-5-cyano-2,3-dihydro-1H-inden-1-amine. HCl;
(S)-4-cyano-2,3-dihydro-1H-inden-1-amine. HCl;
(S)-6,7-dihydro-5H-cyclopenta[b]pyridin-5-amine. HCl;

Similarly, the corresponding (R)-configured amines may be made using the enantiomeric (R,R)-configured sulfinamide starting materials. The amine hydrochlorides may be used in salt form without further purification for preparation of compounds in the present invention or alternatively desalted by partition between DCM and aqueous base, drying the separated organic phase (Na₂SO₄) and then recovering the free base amine by evaporation.

### Examples 1 to 7

Examples 1 to 7 may be prepared according to the route shown in Scheme 1

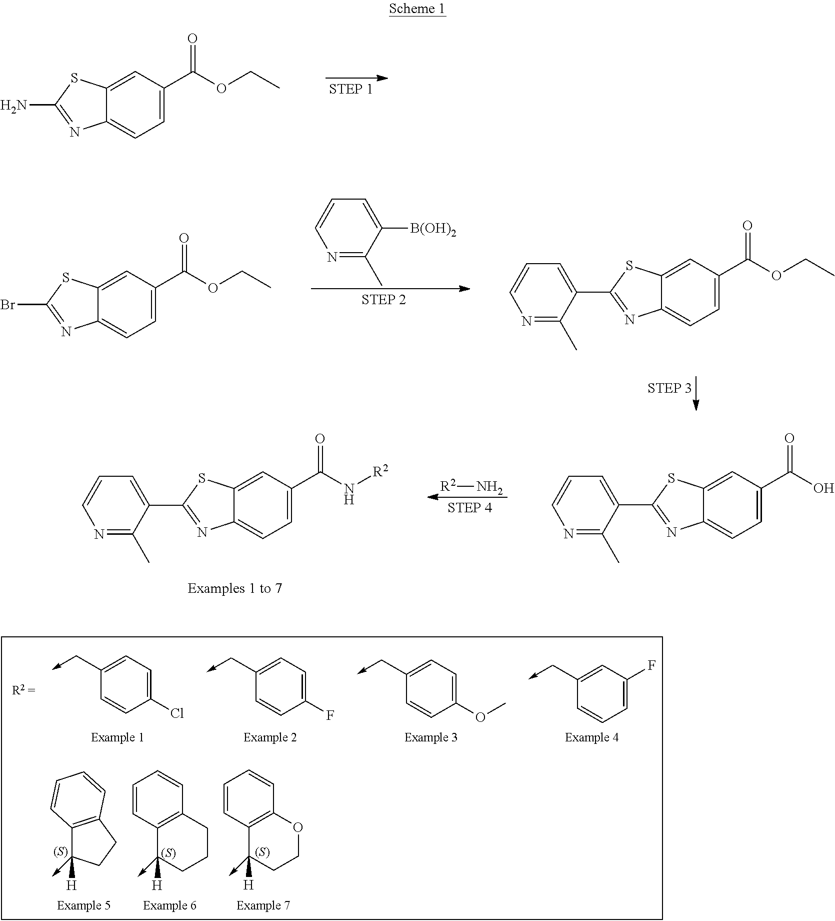

### Step 1 (Scheme 1): Synthesis of ethyl 2-bromobenzo[d]thiazole-6-carboxylate

To an ice-cooled, stirred suspension of copper(II) bromide (1.9 equiv.) in acetonitrile (volume selected to give 0.22 M solution of benzothiazole substrate) was added tert-butyl nitrite (1.9 equiv.). The mixture was brought to ambient temperature, stirring for 30 min, prior to addition of ethyl 2-aminobenzo[d]thiazole-6-carboxylate substrate (1.0 equiv.), stirring at ambient temperature for a further period of 16 h. The mixture was then concentrated in vacuo to afford a residue that was partitioned between EtOAc and 1.5 M hydrochloric acid. The organic phase was washed with brine, dried (Na₂SO₄) and evaporated. The resulting residue was triturated with hexanes to afford ethyl 2-bromobenzo[d]thiazole-6-carboxylate as a brown solid (69% yield).

¹H NMR (400 MHz, DMSO-d₆): δ 8.81 (1H, app. s), 8.11-8.06 (2H, m), 4.37 (2H, q, J6.8 Hz), 1.36 (3H, t, J 6.8 Hz).

### Step 2 (Scheme 1): Synthesis of ethyl 2-(2-methylpyridin-3-yl)benzo[d]thiazole-6-carboxylate

To a stirred, argon-purged mixture of ethyl 2-bromobenzo[d]thiazole-6-carboxylate (1.0 equiv.) in 91% v/v 1,4-dioxane/H₂O (0.34 M in substrate) was added (3-methylpyridin-4-yl)boronic acid (1.1 equiv.), K₂CO₃ (3.0 equiv.) and Pd(PPh₃)₄ (10 mol %). The reaction mixture was heated to reflux under argon for 16 h and then cooled and filtered through Celite®, washing with EtOAc. The filtrate was washed with water followed by brine and then dried (Na₂SO₄) and concentrated under reduced pressure. The crude product was processed by flash column chromatography (30-40% EtOAc/hexanes); fractions containing the target material were combined and evaporated to obtain ethyl 2-(2-methylpyridin-3-yl)benzo[d]thiazole-6-carboxylate as a pale yellow solid (74% yield).

### Step 3 (Scheme 1): Synthesis of 2-(2-methylpyridin-3-yl)benzo[d]thiazole-6-carboxylic acid

To a mixture of ethyl 2-(2-methylpyridin-3-yl)benzo[d]thiazole-6-carboxylate (1.0 equiv.) in 2.3:1 v/v THF/H₂O (0.33 M in substrate) was added LiOH·H₂O (2.0 equiv.), stirring at ambient temperature for 16 h (monitored by TLC). The reaction mixture was concentrated under reduced pressure to afford a residue that was diluted with ice-cold water and acidified with 1.5 N hydrochloric acid. The resulting precipitate was collected by filtration and dried to afford 2-(2-methylpyridin-3-yl)benzo[d]thiazole-6-carboxylic acid as pale yellow solid (95% yield).

### Step 4 (Scheme 1) Leading to Examples 1 to 7: General Amide Coupling Procedure

To a solution of 2-(2-methylpyridin-3-yl)benzo[d]thiazole-6-carboxylic acid (1.0 equiv.) in DMF (0.1 M in substrate) at 0° C. was added EDC.HCl (2.0 equiv.), HOBt (1.5 equiv.), DIPEA (1.0 equiv.) and amine (R²NH₂; 2.0 equiv.), stirring at ambient temperature for 16 h. The reaction mixture was diluted with DCM and washed with water followed by brine. The separated organic layer was then dried (Na₂SO₄) and concentrated under reduced pressure. The crude material was processed by flash chromatography (5-10% MeOH in DCM eluent). Fractions containing the target material were combined and evaporated to dryness. The resulting solid was triturated with diethyl ether and dried to afford Examples 1 to 7 as off-white solids.

### Example 1: N-(4-chlorobenzyl)-2-(2-methylpyridin-3-yl)benzo[d]thiazole-6-carboxamide

Prepared according to Scheme 1 using 4-chlorobenzylamine as the amine component.

¹H NMR (free base form): δ_{H} (400 MHz, DMSO-d₆) 9.26 (1H, br. t, J5.9), 8.73 (1H, app. d, J 1.7), 8.64 (1H, dd, J4.8 and 1.6), 8.25 (1H, dd, J7.9 and 1.6), 8.20 (1H, tented d, J8.6), 8.08 (1H, tented dd, J 8.6 and 1.7), 7.47 (1H, dd, J 7.8 and 4.8), 7.42-7.37 (4H, AA′BB′ m), 4.52 (2H, d, J 5.9), 2.85 (3H, s).

### Example 2: N-(4-fluorobenzyl)-2-(2-methylpyridin-3-yl)benzo[d]thiazole-6-carboxamide

Prepared according to Scheme 1 using 4-fluorobenzylamine as the amine component.

¹H NMR (free base form): δ_{H} (400 MHz, DMSO-d₆) 9.24 (1H, br. t, J5.9), 8.73 (1H, app. d, J 1.7), 8.64 (1H, dd, J4.8 and 1.6), 8.25 (1H, dd, J7.9 and 1.6), 8.19 (1H, tented d, J8.6), 8.08 (1H, tented dd, J 8.6 and 1.7), 7.47 (1H, dd, J7.8 and 4.8), 7.43-7.38 (2H, m), 7.20-7.14 (2H, m), 4.51 (2H, d, J 5.9), 2.85 (3H, s).

### Example 3: N-(4-methoxybenzyl)-2-(2-methylpyridin-3-yl)benzo[d]thiazole-6-carboxamide

Prepared according to Scheme 1 using 4-methoxybenzylamine as the amine component.

¹H NMR (free base form): δ_{H} (400 MHz, DMSO-d₆) 9.16 (1H, br. t, J5.9), 8.71 (1H, app. d, J 1.7), 8.64 (1H, dd, J4.8 and 1.6), 8.25 (1H, dd, J7.9 and 1.6), 8.19 (1H, tented d, J8.6), 8.07 (1H, tented dd, J 8.6 and 1.7), 7.47 (1H, dd, J 7.8 and 4.8), 7.31-7.27 (2H, AA′BB′ m), 6.92-6.89 (2H, AA′BB′ m), 4.46 (2H, d, J 5.9), 3.73 (3H, s), 2.85 (3H, s).

### Example 4: N-(3-fluorobenzyl)-2-(2-methylpyridin-3-yl)benzo[d]thiazole-6-carboxamide

Prepared according to Scheme 1 using 3-fluorobenzylamine as the amine component.

¹H NMR (free base form): δ_{H} (400 MHz, DMSO-d₆) 9.25 (1H, br. t, J5.9), 8.74 (1H, app. d, J 1.7), 8.64 (1H, dd, J4.8 and 1.6), 8.25 (1H, dd, J7.9 and 1.6), 8.20 (1H, tented d, J8.6), 8.09 (1H, tented dd, J 8.6 and 1.7), 7.47 (1H, dd, J7.9 and 4.8), 7.42-7.36 (1H, m), 7.23-7.15 (2H, m), 7.11-7.06 (1H, m), 4.55 (2H, d, J5.9), 2.85 (3H, s).

### Example 5: (S)-N-(2,3-dihydro-1H-inden-1-yl)-2-(2-methylpyridin-3-yl)benzo[d]thiazole-6-carboxamide

Prepared according to Scheme 1 using (S)-1-aminoindane as the amine component.

¹H NMR (free base form): δ_{H} (400 MHz, DMSO-d₆) 8.96 (1H, br. d, J 8.2), 8.75 (1H, app. d, J 1.7), 8.65 (1H, dd, J 4.8 and 1.6), 8.25 (1H, dd, J 7.9, 1.6), 8.19 (1H, tented d, J 8.6), 8.12 (1H, tented dd, J 8.6 and 1.7), 7.47 (1H, dd, J7.8 and 4.8), 7.30-7.18 (4H, m), 5.64-5.58 (1H, m), 3.06-2.99 (1H, m), 2.92-2.84 (4H, m), 2.53-2.45 (1H, m), 2.07-1.98 (1H, m).

### Example 6: (S)-2-(2-methylpyridin-3-yl)-N-(1,2,3,4-tetrahydronaphthalen-1-yl)benzo[d]thiazole-6-carboxamide

Prepared according to Scheme 1 using (S)-1-amino-1,2,3,4-tetrahydronaphthalene as the amine component.

¹H NMR (free base form): δ_{H} (400 MHz, DMSO-d₆) 8.95 (1H, d, J 8.5), 8.75 (1H, app. d, J 1.7), 8.64 (1H, dd, J 4.8, 1.5), 8.25 (1H, dd, J 7.8 and 1.5), 8.18 (1H, tented d, J 8.6), 8.12 (1H, tented dd, J 8.6 and 1.7), 7.47 (1H, dd, J 7.8 and 4.8), 7.25-7.22 (1H, m), 7.20-7.12 (3H, m), 5.32-5.26 (1H, m), 2.85 (3H, s), 2.84-2.72 (2H, m), 2.07-1.95 (2H, m), 1.91-1.73 (2H, m).

### Example 7: (S)-N-(chroman-4-yl)-2-(2-methylpyridin-3-yl)benzo[d]thiazole-6-carboxamide

Prepared according to Scheme 1 using (S)-chroman-4-amine as the amine component.

¹H NMR (free base form): δ_{H} (400 MHz, DMSO-d₆) 9.06 (1H, br. d, J8.1), 8.75 (1H, app. d, J 1.7), 8.64 (1H, dd, J4.7 and 1.3), 8.25 (1H, dd, J7.8 and 1.3), 8.18 (1H, tented d, J8.6), 8.12 (1H, tented dd, J 8.6 and 1.7), 7.47 (1H, dd, J7.8 and 4.8), 7.23 (1H, d, J7.5), 7.18 (1H, app. t, J 7.7), 6.90 (1H, t, J 7.7), 6.83 (1H, d, J 8.2), 5.35-5.30 (1H, m), 4.37-4.23 (2H, m), 2.85 (3H, s), 2.20-2.04 (2H, m).

### Example 8: (S)-2-(pyridin-3-yl)-N-(1,2,3,4-tetrahydronaphthalen-1-yl)benzo[d]thiazole-6-carboxamide

Prepared according to Scheme 1, using pyridin-3-ylboronic acid instead of (2-methylpyridin-3-yl)boronic acid in Step 2 and (S)-1-amino-1,2,3,4-tetrahydronaphthalene as the amine component.

¹H NMR (free base form): δ_{H} (400 MHz, DMSO-d₆) 9.30 (1H, dd, J 2.2 and 0.6), 8.94 (1H, br. d, J 8.5), 8.78 (1H, dd, J 4.8 and 1.6), 8.75 (1H, app. d, J 1.7), 8.49 (1H, ddd, J 8.0, 2.2 and 1.6), 8.17 (1H, tented d, J 8.6), 8.11 (1H, tented dd, J 8.6 and 1.7), 7.64 (1H, ddd, J 8.0, 4.8 and 0.6), 7.26-7.11 (4H, m), 5.31-5.26 (1H, m), 2.88-2.72 (2H, m), 2.07-1.95 (2H, m), 1.92-1.73 (2H, m).

### Example 9: (S)-N-(chroman-4-yl)-2-(pyridin-3-yl)benzo[d]thiazole-6-carboxamide

Prepared according to Scheme 1, using pyridin-3-ylboronic acid instead of (2-methylpyridin-3-yl)boronic acid in Step 2 and (S)-chroman-4-amine as the amine component.

¹H NMR (free base form): δ_{H} (400 MHz, DMSO-d₆) 9.30 (1H, dd, J 2.2 and 0.6), 9.04 (1H, br. d, J 8.1), 8.78 (1H, dd, J 4.8 and 1.6), 8.75 (1H, app. d, J 1.7), 8.49 (1H, ddd, J 8.0, 2.2 and 1.6), 8.17 (1H, tented d, J 8.6), 8.11 (1H, tented dd, J 8.6 and 1.7), 7.64 (1H, ddd, J 8.0, 4.8 and 0.6), 7.23 (1H, app. d, J 7.6), 7.17 (1H, app. td, J 7.7 and 1.5), 6.89 (1H, td, J 7.4 and 1.1), 6.82 (1H, dd, J 8.2 and 1.1), 5.35-5.30 (1H, m), 4.36-4.24 (2H, m), 2.20-2.05 (2H, m).

### Example 10: N-benzyl-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and benzylamine as the amine component.

¹H NMR (free base form): δ_{H} (400 MHz, DMSO-d₆) 9.21 (1H, br. t, J5.8), 9.17 (1H, d, J2.2), 8.71 (1H, app. d, J 1.7), 8.37 (1H, dd, J 8.1 and 2.2), 8.16 (1H, tented d, J 8.6), 8.07 (1H, tented dd, J 8.6 and 1.7), 7.49 (1H, d, J 8.1), 7.39-7.30 (4H, m), 7.30-7.22 (1H, m), 4.53 (2H, d, J5.8), 2.58 (3H, s).

### Example 11: N-(4-chlorobenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 4-chlorobenzylamine as the amine component.

¹H NMR (free base form): δ_{H} (400 MHz, DMSO-d₆) 9.25 (1H, br. t, J 6.0), 9.17 (1H, d, J 2.3), 8.70 (1H, app. d, J 1.7), 8.37 (1H, dd, J 8.1 and 2.3), 8.16 (1H, tented d, J 8.6), 8.06 (1H, tented dd, J 8.6 and 1.7), 7.50 (1H, d, J 8.1), 7.42-7.36 (4H, AA′BB′ m), 4.51 (2H, d, J 6.0), 2.58 (3H, s).

### Example 12: N-(4-fluorobenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 4-fluorobenzylamine as the amine component.

¹H NMR (free base form): δ_{H} (400 MHz, DMSO-d₆) 9.22 (1H, br. t, J 5.9), 9.17 (1H, d, J 2.3), 8.69 (1H, app. d, J 1.7), 8.37 (1H, dd, J 8.1 and 2.3), 8.16 (1H, tented d, J 8.6), 8.06 (1H, tented dd, J 8.6 and 1.7), 7.50 (1H, d, J 8.1), 7.43-7.37 (2H, m), 7.20-7.14 (2H, m), 4.51 (2H, d, J 5.9), 2.58 (3H, s).

### Example 13: N-(4-methoxybenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 4-methoxybenzylamine as the amine component.

¹H NMR (free base form): δ_{H} (400 MHz, DMSO-d₆) 9.17 (1H, d, J 2.3), 9.22 (1H, br. t, J 5.9), 8.69 (1H, app. d, J 1.7), 8.37 (1H, dd, J 8.1 and 2.3), 8.15 (1H, tented d, J 8.6), 8.06 (1H, tented dd, J 8.6 and 1.7), 7.49 (1H, d, J 8.1), 7.30-7.26 (2H, AA′BB′ m), 6.92-7.88 (2H, AA′BB′ m), 4.45 (2H, d, J5.9), 3.73 (3H, s), 2.58 (3H, s).

### Example 14: N-(3-chlorobenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 3-chlorobenzylamine as the amine component.

¹H NMR (free base form): δ_{H} (400 MHz, DMSO-d₆) 9.25 (1H, br. t, J 5.9), 9.17 (1H, d, J 2.3), 8.71 (1H, app. d, J 1.7), 8.37 (1H, dd, J 8.1 and 2.3), 8.16 (1H, tented d, J 8.6), 8.06 (1H, tented dd, J 8.6 and 1.7), 7.49 (1H, d, J 8.1), 7.42-7.31 (4H, m), 4.53 (2H, d, J 5.9), 2.58 (3H, s).

### Example 15: N-(3-fluorobenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 3-fluorobenzylamine as the amine component.

¹H NMR (free base form): δ_{H} (400 MHz, DMSO-d₆) 9.26 (1H, br. t, J 5.3), 9.17 (1H, app. s), 8.71 (1H, app. s), 8.37 (1H, app. d, J 8.1), 8.16 (1H, d, J 8.6), 8.07 (1H, d, J 8.6), 7.49 (1H, d, J 8.1), 7.42-7.35 (1H, m), 7.23-7.14 (2H, m), 7.12-7.05 (1H, m), 4.54 (2H, d, J5.3), 2.58 (3H, s).

### Example 16: N-(3-methoxybenzyl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and 3-methoxybenzylamine as the amine component.

¹H NMR (free base form): δ_{H} (400 MHz, DMSO-d₆) 9.19 (1H, br. t, J5.9), 9.17 (1H, d, J2.3), 8.71 (1H, app. d, J 1.7), 8.37 (1H, dd, J 8.1 and 2.3), 8.16 (1H, tented d, J 8.6), 8.06 (1H, tented dd, J 8.6 and 1.7), 7.49 (1H, d, J 8.1), 7.25 (1H, t, J 8.1), 6.95-6.91 (2H, m), 6.84-6.80 (1H, m), 4.50 (2H, d, J5.9), 3.74 (3H, s), 2.58 (3H, s).

### Example 17: (S)-N-(2,3-dihydro-1H-inden-1-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (S)-1-aminoindane as the amine component.

¹H NMR (free base form): δ_{H} (400 MHz, CD₃OD) 9.15 (1H, d, J 2.3), 8.57 (1H, dd, J 1.7 and 0.5), 8.41 (1H, dd, J 8.1 and 2.3), 8.11 (1H, tented dd, J 8.6 and 0.5), 8.05 (1H, tented dd, J 8.6 and 1.7), 7.51 (1H, d, J 8.1), 7.36-7.18 (4H, m), 5.69 (1H, app. t, J7.8), 3.13-3.06 (1H, m), 2.98-2.92 (1H, m), 2.67-2.59 (1H, m), 2.64 (3H, s), 2.11-2.01 (1H, m). [Amide NH lost to deuterium exchange.]

### Example 18: (R)-N-(2,3-dihydro-1H-inden-1-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (R)-1-aminoindane as the amine component.

¹H NMR (free base form): δ_{H} (400 MHz, DMSO-d₆) 9.17 (1H, d, J 2.3), 8.93 (1H, br. d, J 8.3), 8.72 (1H, app. d, J 1.7), 8.37 (1H, dd, J 8.1 and 2.3), 8.14 (1H, tented d, J 8.6), 8.09 (1H, tented dd, J 8.6 and 1.7), 7.49 (1H, d, J 8.1), 7.32-7.17 (4H, m), 5.64-5.57 (1H, m), 3.06-2.99 (1H, m), 2.91-2.83 (1H, m), 2.58 (3H, s), 2.54-2.45 (1H, m), 2.07-1.97 (1H, m).

### Example 19: N-((1S,2R)-2-hydroxy-2,3-dihydro-1H-inden-1-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (1S,2R)-1-amino-2-indanol as the amine component.

¹H NMR (free base form): δ_{H} (400 MHz, DMSO-d₆) 9.18 (1H, d, J2.3), 8.82 (1H, app. s), 8.49 (1H, br. d, J 8.4), 8.38 (1H, dd, J 8.1 and 2.3), 8.15 (2H, app. s), 7.49 (1H, d, J 8.1), 7.31-7.18 (4H, m), 5.49 (1H, dd, J 8.4 and 5.3), 5.17 (1H, br. d, J 4.6), 4.58-4.53 (1H, m), 3.12 (1H, dd, J 16.1 and 5.0), 2.91 (1H, app. d, J 16.1), 2.58 (3H, s).

### Example 20: N-((1R,2S)-2-hydroxy-2,3-dihydro-1H-inden-1-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (1R,2S)-1-amino-2-indanol as the amine component.

¹H NMR (free base form): δ_{H} (400 MHz, DMSO-d₆) 9.18 (1H, d, J2.3), 8.82 (1H, app. s), 8.48 (1H, br. d, J 8.4), 8.38 (1H, dd, J 8.1 and 2.3), 8.15 (2H, app. s), 7.49 (1H, d, J 8.1), 7.31-7.18 (4H, m), 5.49 (1H, dd, J 8.4 and 5.3), 5.16 (1H, br. d, J 4.6), 4.58-4.53 (1H, m), 3.12 (1H, dd, J 16.1 and 5.0), 2.91 (1H, app. d, J 16.1), 2.58 (3H, s).

### Example 21: N-((1R,2R)-2-hydroxy-2,3-dihydro-1H-inden-1-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (1R,2R)-1-amino-2-indanol as the amine component.

¹H NMR (free base form): δ_{H} (400 MHz, DMSO-d₆) 9.18 (1H, d, J2.3), 8.92 (1H, br. d, J 8.4), 8.74 (1H, app. d, J 1.7), 8.38 (1H, dd, J 8.1 and 2.3), 8.17 (1H, tented d, J 8.6), 8.12 (1H, tented dd, J 8.6 and 1.7), 7.50 (1H, d, J 8.1), 7.25-7.14 (4H, m), 5.39 (1H, d, J 5.7), 5.34 (1H, app. t, J7.6), 4.50-4.43 (1H, m), 3.20 (1H, dd, J 15.4 and 7.2), 2.77 (1H, dd, J 15.4 and 7.7), 2.58 (3H, s).

### Example 22: N-cyclopentyl-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and cyclopentylamine as the amine component.

¹H NMR (free base form): δ_{H} (400 MHz, DMSO-d₆) 9.17 (1H, d, J2.3), 8.65 (1H, app. d, J 1.7), 8.47 (1H, br. d, J7.2), 8.37 (1H, dd, J8.1 and 2.3), 8.13 (1H, tented d, J8.6), 8.02 (1H, tented dd, J 8.6 and 1.7), 7.49 (1H, d, J 8.1), 4.31-4.22 (1H, m), 2.58 (3H, s), 1.97-1.86 (2H, m), 1.77-1.66 (2H, m), 1.63-1.50 (4H, m).

### Example 23: (S)-2-(6-methylpyridin-3-yl)-N-(1,2,3,4-tetrahydronaphthalen-1-yl)benzo[d]thiazole-6-carboxamide

Prepared according to Scheme 1, using (6-methylpyridin-3-yl)boronic acid instead of (2-methylpyridin-3-yl)boronic acid in Step 2 and (S)-1-amino-1,2,3,4-tetrahydronaphthalene as the amine component.

¹H NMR (free base form): δ_{H} (400 MHz, DMSO-d₆) 9.17 (1H, d, J 2.3), 8.92 (1H, br. d, J 8.5), 8.73 (1H, app. d, J 1.7), 8.36 (1H, dd, J 8.1 and 2.3), 8.14 (1H, tented d, J 8.6), 8.09 (1H, tented dd, J 8.6 and 1.7), 7.49 (1H, d, J 8.1), 7.26-7.21 (1H, m), 7.20-7.11 (3H, m), 5.31-5.26 (1H, m), 2.88-2.72 (2H, m), 2.58 (3H, s), 2.07-1.94 (2H, m), 1.92-1.73 (2H, m).

### Example 24: (S)-N-(chroman-4-yl)-2-(6-methylpyridin-3-yl)benzo[d]thiazole-6-carboxamide

Prepared according to Scheme 1, using (6-methylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.17 (1H, d, J 2.3), 9.03 (1H, br. d, J 8.1), 8.74 (1H, app. d, J 1.7), 8.36 (1H, dd, J 8.1 and 2.3), 8.14 (11H, tented d, J 8.6), 8.09 (1H, tented dd, J8.6 and 1.7), 7.49 (1H, d, J8.1), 7.23 (1H, app. d, J7.6), 7.17 (1H, app. td, J7.7 and 1.5), 6.89 (1H, td, J7.4 and 1.1), 6.82 (1H, dd, J 8.2 and 1.1), 5.35-5.30 (1H, m), 4.36-4.23 (2H, m), 2.58 (3H, s), 2.20-2.04 (2H, m).

### Example 25: (S)-N-(chroman-4-yl)-2-(6-(trifluoromethyl)pyridin-3-yl)benzo[d]thiazole-6-carboxamide

Prepared according to Scheme 1, using (6-(trifluoromethyl)pyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.48 (1H, d, J 2.0), 9.07 (1H, d, J 8.1), 8.80 (1H, app. d, J 1.7), 9.77 (1H, dd, J 8.1 and 2.0), 8.23 (1H, tented d, J 8.6), 8.15-8.11 (2H, m), 7.23 (1H, app. d, J7.6), 7.18 (1H, app. td, J7.7 and 1.5), 6.89 (1H, td, J7.4 and 1.1), 6.82 (1H, dd, J 8.2 and 1.1), 5.35-5.30 (1H, m), 4.36-4.24 (2H, m), 2.21-2.04 (2H, m).

### Example 26: (S)-N-(chroman-4-yl)-2-(2,6-dimethylpyridin-3-yl)benzo[d]thiazole-6-carboxamide

Prepared according to Scheme 1, using (2,6-dimethylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.04 (1H, br. d, J8.1), 8.74 (1H, app. d, J 1.7), 8.17-8.09 (3H, m), 7.32 (1H, d, J8.1), 7.22 (1H, app. d, J7.6), 7.18 (1H, app. td, J7.7 and 1.5), 6.89 (1H, td, J7.4 and 1.1), 6.82 (1H, dd, J 8.2 and 1.1), 5.35-5.30 (1H, m), 4.36-4.24 (2H, m), 2.82 (3H, s), 2.53 (3H, s), 2.20-2.04 (2H, m).

### Example 27: (S)-N-(chroman-4-yl)-2-(6-cyclopropylpyridin-3-yl)benzo[d]thiazole-6-carboxamide

Prepared according to Scheme 1, using (6-cyclopropylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.11 (1H, d, J 2.3), 9.03 (1H, br. d, J 8.1), 8.71 (1H, app. d, J 1.7), 8.32 (1H, dd, J 8.2 and 2.3), 8.13 (1H, tented d, J 8.6), 8.08 (1H, tented dd, J8.6 and 1.7), 7.53 (1H, d, J8.2), 7.22 (1H, app. d, J7.6), 7.17 (1H, app. td, J7.7 and 1.5), 6.89 (1H, td, J7.4 and 1.1), 6.82 (1H, dd, J 8.2 and 1.1), 5.34-5.29 (1H, m), 4.36-4.23 (2H, m), 2.27-2.21 (1H, m), 2.20-2.04 (2H, m), 1.10-1.01 (4H, m).

### Example 28: (S)-N-(chroman-4-yl)-2-(6-isopropylpyridin-3-yl)benzo[d]thiazole-6-carboxamide

Prepared according to Scheme 1, using (6-isopropylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.21 (1H, d, J 2.3), 9.02 (1H, br. d, J 8.0), 8.73 (1H, app. d, J 1.7), 8.40 (1H, dd, J 8.1 and 2.3), 8.15 (1H, tented d, J 8.6), 8.09 (1H, tented dd, J 8.6 and 1.7), 7.52 (1H, d, J 8.1), 7.23 (1H, app. d, J7.6), 7.17 (1H, app. td, J7.7 and 1.5), 6.89 (1H, td, J7.4 and 1.1), 6.82 (1H, dd, J 8.2 and 1.1), 5.35-5.30 (1H, m), 4.36-4.23 (2H, m), 3.14 (1H, septet, J 6.9), 2.20-2.04 (2H, m), 1.29 (6H, d, J 6.9).

### Example 29: (S)-N-(chroman-4-yl)-2-(6-ethylpyridin-3-yl)benzo[d]thiazole-6-carboxamide

Prepared according to Scheme 1, using (6-ethylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.20 (1H, d, J 2.3), 9.03 (1H, br. d, J 8.1), 8.73 (1H, app. d, J 1.7), 8.39 (1H, dd, J 8.2 and 2.3), 8.15 (1H, tented d, J 8.6), 8.09 (1H, tented dd, J 8.6 and 1.7), 7.50 (1H, d, J 8.2), 7.23 (1H, app. d, J7.6), 7.17 (1H, app. td, J7.7 and 1.5), 6.89 (1H, td, J7.4 and 1.1), 6.82 (1H, dd, J 8.2 and 1.1), 5.35-5.30 (1H, m), 4.36-4.23 (2H, m), 2.86 (2H, q, J7.6), 2.20-2.04 (2H, m), 1.28 (3H, t, J7.6).

### Example 30: N-benzyl-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and benzylamine as the amine component.

¹H NMR (free base form): δ_{H} (300 MHz, DMSO-d₆) 9.23 (1H, br. t, J 5.9), 9.10 (1H, d, J 2.0), 8.71 (1H, app. d, J 1.7), 8.62 (1H, app. d, J2.0), 8.33 (1H, tq, J2.0 and 0.7), 8.17 (1H, tented d, J 8.6), 8.07 (1H, tented dd, J 8.6 and 1.7), 7.41-7.21 (5H, m), 4.53 (2H, d, J 5.9), 2.44 (3H, app. s).

### Example 31: N-(4-chlorobenzyl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 4-chlorobenzylamine as the amine component.

¹H NMR (free base form): δ_{H} (400 MHz, DMSO-d₆) 9.23 (1H, br. t, J 5.9), 9.10 (1H, d, J 2.0), 8.72 (1H, app. d, J 1.7), 8.62 (1H, app. d, J2.0), 8.32 (1H, tq, J2.0 and 0.7), 8.17 (1H, tented d, J 8.6), 8.07 (1H, tented dd, J 8.6 and 1.7), 7.42-7.36 (4H, AA′BB′ m), 4.51 (2H, d, J 5.9), 2.44 (3H, app. s).

### Example 32: N-(4-fluorobenzyl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 4-fluorobenzylamine as the amine component.

¹H NMR (free base form): δ_{H} (400 MHz, DMSO-d₆) 9.22 (1H, br. t, J 5.9), 9.11 (1H, d, J 2.0), 8.72 (1H, app. d, J 1.7), 8.63 (1H, app. d, J2.0), 8.32 (1H, tq, J2.0 and 0.7), 8.17 (1H, tented d, J 8.6), 8.07 (1H, tented dd, J 8.6 and 1.7), 7.43-7.37 (2H, m), 7.20-7.14 (2H, m), 4.51 (2H, d, J5.9), 2.44 (3H, app. s).

### Example 33: N-(4-methoxybenzyl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 4-methoxybenzylamine as the amine component.

¹H NMR (free base form): δ_{H} (400 MHz, DMSO-d₆) 9.15 (1H, br. t, J 5.9), 9.10 (1H, d, J 2.0), 8.71 (1H, app. d, J 1.7), 8.63 (1H, app. d, J2.0), 8.32 (1H, tq, J2.0 and 0.7), 8.16 (1H, tented d, J 8.6), 8.06 (1H, tented dd, J 8.6 and 1.7), 7.31-7.27 (2H, AA′BB′ m), 6.92-6.88 (2H, AA′BB′ m), 4.45 (2H, d, J5.9), 3.73 (3H, s), 2.44 (3H, app. s).

### Example 34: N-[(3-chlorophenyl)methyl]-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 3-chlorobenzylamine as the amine component.

¹H NMR (free base form): δ_{H} (400 MHz, DMSO-d₆) 9.26 (1H, br. t, J 5.9), 9.11 (1H, d, J 2.0), 8.73 (1H, app. d, J 1.7), 8.63 (1H, app. d, J 1.8), 8.33 (1H, tq, J2.0 and 0.7), 8.18 (1H, tented d, J 8.6), 8.07 (1H, tented dd, J 8.6 and 1.7), 7.43-7.28 (4H, m), 4.53 (2H, d, J 5.9), 2.44 (3H, app. s).

### Example 35: N-(3-fluorobenzyl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 3-fluorobenzylamine as the amine component.

¹H NMR (free base form): δ_{H} (400 MHz, DMSO-d₆) 9.25 (1H, br. t, J 5.9), 9.11 (1H, d, J 2.0), 8.73 (1H, app. d, J 1.7), 8.63 (1H, app. d, J2.0), 8.32 (1H, tq, J2.0 and 0.7), 8.18 (1H, tented d, J 8.6), 8.08 (1H, tented dd, J 8.6 and 1.7), 7.42-7.36 (1H, m), 7.22-7.15 (2H, m), 7.11-7.06 (1H, m), 4.54 (2H, d, J5.9), 2.44 (3H, app. s).

### Example 36: N-(3-methoxybenzyl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and 3-methoxybenzylamine as the amine component.

¹H NMR (free base form): δ_{H} (400 MHz, DMSO-d₆) 9.19 (1H, br. t, J5.9), 9.11 (1H, d, J2.0), 8.72 (1H, app. d, J 1.7), 8.63 (1H, app. d, J2.0), 8.32 (1H, tq, J2.0 and 0.7), 8.17 (1H, tented d, J 8.6), 8.07 (1H, tented dd, J 8.6 and 1.7), 7.26 (1H, t, J 8.1), 6.96-6.91 (2H, m), 6.85-6.81 (1H, m), 4.50 (2H, d, J5.9), 3.74 (3H, s), 2.44 (3H, app. s).

### Example 37: (S)-N-(2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (S)-1-aminoindane as the amine component.

¹H NMR (free base form): δ_{H} (400 MHz, DMSO-d₆) 9.11 (1H, d, J 2.0), 8.94 (1H, br. d, J 8.2), 8.74 (1H, app. d, J 1.7), 8.62 (1H, app. d, J2.0), 8.32 (1H, tq, J2.0 and 0.7), 8.16 (1H, tented d, J 8.6), 8.10 (1H, tented dd, J 8.6 and 1.7), 7.31-7.17 (4H, m), 5.64-5.57 (1H, m), 3.03 (1H, tented ddd, J 15.9, 8.9 and 3.2), 2.88 (1H, tented dt, J 15.9 and 8.4), 2.53-2.44 (1H, m), 2.44 (3H, app. s), 2.03 (1H, dq, J 12.5 and 8.4).

### Example 38: (R)-N-(2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (R)-1-aminoindane as the amine component.

¹H NMR (free base form): δ_{H} (400 MHz, DMSO-d₆) 9.11 (1H, d, J 2.0), 8.95 (1H, br. d, J 8.3), 8.74 (1H, app. d, J 1.7), 8.62 (1H, app. d, J2.0), 8.32 (1H, tq, J2.0 and 0.7), 8.16 (1H, tented d, J 8.6), 8.10 (1H, tented dd, J 8.6 and 1.7), 7.31-7.17 (4H, m), 5.64-5.57 (1H, m), 3.02 (1H, tented ddd, J 15.9, 8.9 and 3.2), 2.87 (1H, tented dt, J 15.9 and 8.4), 2.53-2.44 (1H, m), 2.44 (3H, app. s), 2.02 (1H, dq, J 12.5 and 8.4).

### Example 39: N-((1R,2S)-2-hydroxy-2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (1R,2S)-1-amino-2-indanol as the amine component.

¹H NMR (free base form): δ_{H} (400 MHz, DMSO-d₆) 9.11 (1H, d, J 2.0), 8.84-8.83 (1H, m), 8.63 (1H, app. d, J 2.0), 8.48 (1H, br. d, J 8.6), 8.33 (1H, tq, J 2.0 and 0.7), 8.18-8.13 (2H, m), 7.31-7.18 (4H, m), 5.50 (1H, dd, J 8.6 and 5.2), 5.15 (1H, br. d, J 4.5), 4.58-4.54 (1H, m), 3.13 (1H, dd, J 16.2 and 5.1), 2.91 (1H, dd, J 16.2 and 1.7), 2.44 (3H, app. s).

### Example 40: N-((1S,2R)-2-hydroxy-2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (1S,2R)-1-amino-2-indanol as the amine component.

¹H NMR (free base form): δ_{H} (400 MHz, DMSO-d₆) 9.11 (1H, d, J2.0), 8.84-8.83 (1H, m), 8.63 (1H, app. d, J 2.0), 8.48 (1H, br. d, J 8.6), 8.33 (1H, tq, J 2.0 and 0.7), 8.18-8.13 (2H, m), 7.31-7.18 (4H, m), 5.50 (1H, dd, J 8.6 and 5.2), 5.15 (1H, br. d, J 4.1), 4.58-4.54 (1H, m), 3.13 (1H, dd, J 16.2 and 5.1), 2.91 (1H, dd, J 16.2 and 1.7), 2.44 (3H, app. s).

### Example 41: N-((1S,2S)-2-hydroxy-2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (1S,2S)-1-amino-2-indanol as the amine component.

¹H NMR (free base form): δ_{H} (400 MHz, DMSO-d₆) 9.11 (1H, d, J 2.0), 8.94 (1H, br. d, J 8.4), 8.76 (1H, dd, J 1.7 and 0.5), 8.63 (1H, dq, J 2.0 and 0.7), 8.33 (1H, tq, J 2.0 and 0.7), 8.18 (1H, tented dd, J 8.6 and 0.5), 8.13 (1H, tented dd, J 8.6 and 1.7), 7.25-7.14 (4H, m), 5.41 (1H, br. d, J5.6), 5.36-5.32 (1H, m), 4.50-4.43 (1H, m), 3.20 (1H, dd, J 15.5 and 7.2), 2.77 (1H, dd, J 15.5 and 7.7), 2.44 (3H, app. s).

### Example 42: N-((1R,2R)-2-hydroxy-2,3-dihydro-1H-inden-1-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (1R,2R)-1-amino-2-indanol as the amine component.

¹H NMR (free base form): δ_{H} (400 MHz, DMSO-d₆) 9.11 (1H, d, J2.0), 8.93 (1H, br. d, J 8.4), 8.76 (1H, dd, J 1.7 and 0.5), 8.63 (1H, dq, J 2.0 and 0.7), 8.33 (1H, tq, J 2.0 and 0.7), 8.18 (1H, tented dd, J 8.6 and 0.5), 8.13 (1H, tented dd, J 8.6 and 1.7), 7.25-7.14 (4H, m), 5.40 (1H, br. d, J5.7), 5.36-5.32 (1H, m), 4.50-4.43 (1H, m), 3.20 (1H, dd, J 15.5 and 7.2), 2.77 (1H, dd, J 15.5 and 7.7), 2.44 (3H, app. s)

### Example 43: N-cyclopentyl-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and cyclopentylamine as the amine component.

¹H NMR (free base form): 5H (40O MHz, DMSO-d₆) 9.10 (1H, d, J2.0), 8.66 (1H, dd, J1.7 and 0.4), 8.62 (1H, app. d, J 2.0), 8.47 (1H, br. d, J7.2), 8.31 (1H, tq, J 2.0 and 0.7), 8.14 (1H, tented dd, J 8.6 and 0.4), 8.02 (1H, tented dd, J 8.6 and 1.7), 4.31-4.22 (1H, m), 2.44 (3H, app. s), 1.97-1.85 (2H, m), 1.78-1.66 (2H, m), 1.62-1.50 (4H, m).

### Example 44: (S)-2-(5-methylpyridin-3-yl)-N-(1,2,3,4-tetrahydronaphthalen-1-yl)benzo[d]thiazole-6-carboxamide

Prepared according to Scheme 1, using (5-methylpyridin-3-yl)boronic acid instead of (2-methylpyridin-3-yl)boronic acid in Step 2 and (S)-1-amino-1,2,3,4-tetrahydronaphthalene as the amine component.

¹H NMR (free base form): δ_{H} (400 MHz, DMSO-d₆) 9.10 (1H, d, J 2.0), 8.94 (1H, br. d, J 8.8), 8.74 (1H, app. d, J 1.7), 8.62 (1H, app. d, J2.0), 8.32 (1H, tq, J2.0 and 0.7), 8.15 (1H, tented d, J 8.6), 8.10 (1H, tented dd, J 8.6 and 1.7), 7.27-7.11 (4H, m), 5.32-5.25 (1H, m), 2.88-2.72 (2H, m), 2.44 (3H, s), 2.07-1.95 (2H, m), 1.92-1.73 (2H, m).

### Example 45: (S)-N-(chroman-4-yl)-2-(5-methylpyridin-3-yl)benzo[d]thiazole-6-carboxamide

Prepared according to Scheme 1, using (5-methylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.10 (1H, d, J 2.0), 9.04 (1H, d, J 8.1), 8.75 (1H, app. d, J 1.7), 8.62 (1H, app. d, J2.0), 8.31 (1H, tq, J2.0 and 0.7), 8.16 (1H, tented d, J 8.6), 8.10 (1H, tented dd, J 8.6 and 1.7), 7.23 (1H, app. d, J7.6), 7.17 (1H, app. td, J7.7 and 1.5), 6.89 (1H, td, J7.5 and 1.1), 6.82 (1H, dd, J 8.2 and 1.1), 5.35-5.30 (1H, m), 4.36-4.24 (2H, m), 2.44 (3H, s), 2.21-2.04 (2H, m).

### Example 46: 2-(5-methylpyridin-3-yl)-N-(tetrahydro-2H-pyran-4-yl)benzo[d]thiazole-6-carboxamide

Prepared according to Scheme 1, using (5-methylpyridin-3-yl)boronic acid instead of (2-methylpyridin-3-yl)boronic acid in Step 2 and 4-aminotetrahydropyran as the amine component. 1H NMR (free base form): δ_{H} (400 MHz, DMSO-d₆) 9.10 (1H, d, J2.0), 8.67 (1H, app. d, J 1.7), 8.62 (1H, app. d, J 2.0), 8.48 (1H, br. d, J7.6), 8.31 (1H, tq, J2.0 and 0.7), 8.15 (1H, tented d, J 8.6), 8.03 (1H, tented dd, J 8.6 and 1.7), 4.09-4.00 (1H, m), 3.93-3.87 (2H, m), 3.41 (2H, td, J 11.6 and 2.1), 2.44 (3H, app. s), 1.84-1.77 (2H, m), 1.67-1.56 (2H, m).

### Example 47: (S)-N-(chroman-4-yl)-2-(5-(trifluoromethyl)pyridin-3-yl)benzo[d]thiazole-6-carboxamide

Prepared according to Scheme 1, using (5-(trifluoromethyl)pyridin-3-yl)boronic acid instead of (2-methylpyridin-3-yl)boronic acid in Step 2 and (S)-chroman-4-amine as the amine component.

¹H NMR (free base form): 5H (400 MHz, DMSO-d₆) 9.58 (1H, d, J 2.0), 9.21 (1H, dq, J 2.0 and 0.9), 9.07 (1H, br. d, J 8.1), 8.80-8.87 (2H, m), 8.22 (1H, tented d, J 8.6), 8.13 (1H, tented dd, J 8.6 and 1.7), 7.23 (1H, app. d, J 7.6), 7.18 (1H, app. td, J 7.7 and 1.5), 6.89 (1H, td, J 7.5 and 1.1), 6.82 (1H, dd, J 8.2 and 1.1), 5.35-5.30 (1H, m), 4.36-4.24 (2H, m), 2.21-2.04 (2H, m).

### Example 48: (S)-N-(chroman-4-yl)-2-(5-cyclopropylpyridin-3-yl)benzo[d]thiazole-6-carboxamide

Prepared according to Scheme 1, using (5-cyclopropylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.06 (1H, d, J 2.1), 9.05 (1H, br. d, J7.8), 8.74 (1H, app. d, J 1.7), 8.59 (1H, d, J2.1), 8.17 (1H, tented d, J 8.6), 8.10 (1H, tented dd, J 8.6 and 1.7), 8.07 (1H, t, J 2.1), 7.23 (1H, app. d, J 7.6), 7.17 (1H, app. td, J 7.7 and 1.5), 6.89 (1H, td, J7.5 and 1.1), 6.82 (1H, dd, J 8.2 and 1.1), 5.35-5.30 (1H, m), 4.36-4.23 (2H, m), 2.21-2.04 (3H, m), 1.12-1.07 (2H, m), 0.93-0.89 (2H, m).

### Example 49: (S)-N-(chroman-4-yl)-2-(5-isopropylpyridin-3-yl)benzo[d]thiazole-6-carboxamide

Prepared according to Scheme 1, using (5-isopropylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.12 (1H, d, J2.1), 9.05 (1H, d, J8.1), 8.75 (1H, app. d, J 1.7), 8.71 (1H, d, J2.1), 8.32 (1H, t, J2.1), 8.18 (1H, tented d, J 8.6), 8.10 (1H, tented dd, J 8.6 and 1.7), 7.23 (1H, app. d, J7.6), 7.17 (1H, app. td, J7.7 and 1.5), 6.89 (1H, td, J7.5 and 1.1), 6.82 (1H, dd, J 8.2 and 1.1), 5.35-5.30 (1H, m), 4.36-4.23 (2H, m), 3.11 (1H, septet, J7.0), 2.21-2.04 (2H, m), 1.33 (6H, d, J7.0 Hz).

### Example 50: (S)-2-(4-methylpyridin-3-yl)-N-(1,2,3,4-tetrahydronaphthalen-1-yl)benzo[d]thiazole-6-carboxamide

Prepared according to Scheme 1, using (4-methylpyridin-3-yl)boronic acid instead of (2-methylpyridin-3-yl)boronic acid in Step 2 and (S)-1-amino-1,2,3,4-tetrahydronaphthalene as the amine component.

¹H NMR (free base form): δ_{H} (400 MHz, DMSO-d₆) 9.00 (1H, s), 8.96 (1H, d, J 8.5), 8.75 (1H, app. d, J 1.5), 8.60 (1H, d, J 5.0), 8.18 (1H, tented d, J 8.6), 8.12 (1H, tented dd, J 8.6 and 1.5), 7.50 (1H, d, J 5.0), 7.27-7.11 (4H, m), 5.32-5.26 (1H, m), 2.89-2.69 (2H, m), 2.68 (3H, s), 2.08-1.94 (2H, m), 1.93-1.73 (2H, m).

### Example 51: (S)-N-(chroman-4-yl)-2-(4-methylpyridin-3-yl)benzo[d]thiazole-6-carboxamide

Prepared according to Scheme 1, using (4-methylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.04 (1H, br. d, J8.1), 9.00 (1H, s), 8.76 (1H, app. d, J 1.7), 8.60 (1H, d, J 5.0), 8.19 (1H, tented d, J 8.6), 8.12 (1H, tented dd, J 8.6 and 1.7), 7.50 (1H, d, J 5.0), 7.23 (1H, app. d, J7.6), 7.17 (1H, app. td, J7.7 and 1.5), 6.89 (1H, td, J 7.5 and 1.1), 6.82 (1H, dd, J 8.2 and 1.1), 5.35-5.30 (1H, m), 4.36-4.23 (2H, m), 2.68 (3H, s), 2.21-2.04 (2H, m).

### Example 52: (S)-N-(chroman-4-yl)-2-(2,4-dimethylpyridin-3-yl)benzo[d]thiazole-6-carboxamide

Prepared according to Scheme 1, using (2,4-dimethylpyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆/D₂O) 8.70 (1H, app. d, J 1.7), 8.45 (1H, d, J 5.1), 8.17 (1H, tented d, J 8.6), 8.08 (1H, tented dd, J 8.6 and 1.7), 7.30 (1H, d, J5.1), 7.22-7.13 (2H, m), 6.89 (1H, td, J 7.5 and 1.1), 6.82 (1H, dd, J 8.2 and 1.1), 5.30 (1H, app. t, J 6.1), 4.34-4.20 (2H, m), 2.30 (3H, s), 2.20-2.03 (5H, m). [Amide NH lost to deuterium exchange.]

### Example 53: (S)-2-(5-chloropyridin-3-yl)-N-(chroman-4-yl)benzo[d]thiazole-6-carboxamide

Prepared according to Scheme 1, using (5-chloropyridin-3-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.25 (1H, d, J 1.9), 9.06 (1H, br. d, J 8.1), 8.85 (1H, d, J2.3), 8.78 (1H, app. d, J 1.7), 8.60 (1H, app. t, J2.1), 8.19 (1H, tented d, J8.6), 8.12 (1H, tented dd, J 8.6 and 1.7), 7.23 (1H, app. d, J7.6), 7.18 (1H, app. td, J7.7 and 1.5), 6.89 (1H, td, J7.5 and 1.1), 6.82 (1H, dd, J8.2 and 1.1), 5.35-5.30 (1H, m), 4.36-4.24 (2H, m), 2.21-2.04 (2H, m).

### Example 54: (S)-N-(chroman-4-yl)-2-(pyridin-4-yl)benzo[d]thiazole-6-carboxamide

Prepared according to Scheme 1, using (pyridin-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.06 (1H, br. d, J 8.1), 8.82-8.80 (2H, m), 8.78 (1H, app. d, J1.7), 8.21 (1H, tented d, J 8.6), 8.12 (1H, tented dd, J 8.6 and 1.7), 8.07-8.06 (2H, m), 7.23 (1H, app. d, J7.6), 7.18 (1H, app. td, J7.7 and 1.5), 6.89 (1H, td, J7.5 and 1.1), 6.82 (1H, dd, J 8.2 and 1.1), 5.35-5.30 (1H, m), 4.36-4.24 (2H, m), 2.21-2.04 (2H, m).

### Example 55: (S)-N-(chroman-4-yl)-2-(3-methylpyridin-4-yl)benzo[d]thiazole-6-carboxamide

Prepared according to Scheme 1, using (3-methylpyridin-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.07 (1H, br. d, J8.1), 8.78 (1H, app. d, J 1.7), 8.70 (1H, s), 8.63 (1H, d, J5.1), 8.22 (1H, tented d, J8.6), 8.13 (1H, tented dd, J8.6 and 1.7), 7.87 (1H, d, J 5.1), 7.23 (1H, app. d, J7.6), 7.18 (1H, app. td, J7.7 and 1.5), 6.89 (1H, td, J7.5 and 1.1), 6.82 (1H, dd, J 8.2 and 1.1), 5.35-5.30 (1H, m), 4.36-4.24 (2H, m), 2.67 (3H, s), 2.21-2.04 (2H, m).

### Example 56: (S)-N-(chroman-4-yl)-2-(2-methylpyridin-4-yl)benzo[d]thiazole-6-carboxamide

Prepared according to Scheme 1, using (2-methylpyridin-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.06 (1H, br. d, J8.1), 8.77 (1H, app. d, J 1.7), 8.67 (1H, d, J5.2), 8.19 (1H, tented d, J 8.6), 8.12 (1H, tented dd, J 8.6 and 1.7), 7.95-7.93 (1H, m), 7.86 (1H, dd, J 5.2 and 1.6), 7.23 (1H, app. d, J 7.6), 7.18 (1H, app. td, J 7.7 and 1.5), 6.89 (1H, td, J7.5 and 1.1), 6.82 (1H, dd, J 8.2 and 1.1), 5.35-5.30 (1H, m), 4.36-4.24 (2H, m), 2.61 (3H, s), 2.21-2.04 (2H, m).

### Example 57: (S)-N-(chroman-4-yl)-2-(1-methyl-1H-pyrazol-4-yl)benzo[d]thiazole-6-carboxamide

Prepared according to Scheme 1, using (1-methyl-1H-pyrazol-4-yl)boronic acid instead of (2-methylpyridin-3-yl)boronic acid in Step 2 and (S)-chroman-4-amine as the amine derivative.

¹H NMR (free base form): δ_{H} (400 MHz, DMSO-d₆) 8.96 (1H, br. d, J8.1), 8.62 (1H, app. d, J 1.6), 8.56 (s, 1H), 8.10 (s, 1H), 8.03 (1H, tented dd, J 8.6 and 1.7), 7.97 (1H, tented d, J 8.6), 7.21 (1H, app. d, J 7.6), 7.17 (1H, app. td, J 7.7 and 1.5), 6.88 (1H, td, J 7.5 and 1.1), 6.81 (1H, dd, J 8.2 and 1.1), 5.34-5.27 (1H, m), 4.35-4.23 (2H, m), 3.93 (3H, s), 2.20-2.03 (2H, m).

### Example 58: (S)-N-(chroman-4-yl)-2-(1,4-dimethyl-1H-pyrazol-5-yl)benzo[d]thiazole-6-carboxamide

Prepared according to Scheme 1, using (1,4-dimethyl-1H-pyrazol-5-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 9.04 (1H, br. d, J8.1), 8.76 (1H, app. d, J 1.7), 8.17 (1H, tented d, J 8.6), 8.11 (1H, tented dd, J 8.6 and 1.7), 7.51 (1H, s), 7.22 (1H, app. d, J7.6), 7.17 (1H, app. td, J7.7 and 1.5), 6.89 (1H, td, J7.5 and 1.1), 6.82 (1H, dd, J 8.2 and 1.1), 5.35-5.30 (1H, m), 4.36-4.24 (2H, m), 2.24 (3H, s), 2.35 (3H, s), 2.20-2.04 (2H, m).

### Example 59: (S)-N-(chroman-4-yl)-2-(1,3,5-trimethyl-1H-pyrazol-4-yl)benzo[d]thiazoIe-6-carboxamide

Prepared according to Scheme 1, using (1,3,5-trimethyl-1H-pyrazol-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 8.97 (1H, br. d, J 8.1), 8.64 (1H, app. dd, J 1.7 and 0.5), 8.04 (1H, tented dd, J 8.6 and 1.7), 8.00 (1H, tented dd, J 8.6 and 0.5), 7.21 (1H, app. d, J7.6), 7.17 (1H, app. td, J7.7 and 1.5), 6.88 (1H, td, J7.5 and 1.1), 6.81 (1H, dd, J 8.2 and 1.1), 5.34-5.29 (1H, m), 4.36-4.23 (2H, m), 3.77 (3H, s), 2.66 (3H, s), 2.47 (3H, s), 2.20-2.03 (2H, m).

### Example 60: (S)-N-(chroman-4-yl)-2-(1,5-dimethyl-1H-pyrazol-4-yl)benzo[d]thiazole-6-carboxamide

Prepared according to Scheme 1, using (1,5-dimethyl-1H-pyrazol-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 8.97 (1H, br. d, J8.1), 8.62 (1H, app. d, J 1.7), 8.48 (1H, s), 8.03 (1H, tented dd, J 8.6 and 1.7), 7.97 (1H, tented d, J 8.6), 7.21 (1H, app. d, J7.6), 7.17 (1H, app. td, J7.7 and 1.5), 6.88 (1H, td, J7.5 and 1.1), 6.81 (1H, dd, J 8.2 and 1.1), 5.34-5.29 (1H, m), 4.35-4.23 (2H, m), 3.85 (3H, s), 2.52 (3H, s), 2.20-2.03 (2H, m).

### Example 61: (S)-N-(chroman-4-yl)-2-(1,3-dimethyl-1H-pyrazol-4-yl)benzo[d]thiazole-6-carboxamide

Prepared according to Scheme 1, using (1,3-dimethyl-1H-pyrazol-4-yl)boronic acid instead of (2-methylpyridin-3-yl)boronic acid in Step 2 and (S)-chroman-4-amine as the amine component.

¹H NMR (free base form): δ_{H} (400 MHz, DMSO-d₆) 8.97 (1H, d, J 8.1), 8.62 (1H, app. d, J 1.7), 8.03 (1H, tented dd, J 8.6 and 1.7), 8.01 (1H, s), 7.98 (1H, tented d, J 8.6), 7.21 (1H, app. d, J7.6), 7.17 (1H, app. td, J7.7 and 1.5), 6.88 (1H, td, J7.5 and 1.1), 6.81 (1H, dd, J 8.2 and 1.1), 5.34-5.29 (1H, m), 4.35-4.23 (2H, m), 3.84 (3H, s), 2.70 (3H, s), 2.20-2.03 (2H, m).

### Example 62: (S)-N-(chroman-4-yl)-2-(5-(hydroxymethyl)pyridin-3-yl)benzo[d]thiazole-6-carboxamide

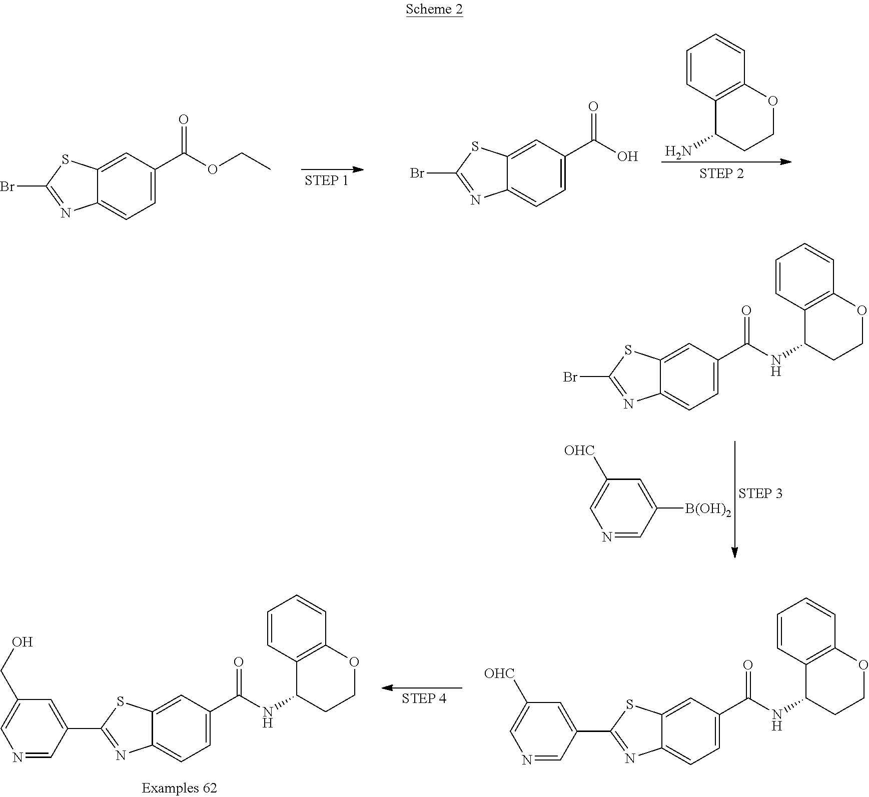

### Step 1 (Scheme 2): Ester hydrolysis

To a stirred mixture of ethyl 2-bromobenzo[d]thiazole-6-carboxylate (prepared as in Scheme 1, Step 1) (1 equiv.) in 2.3:1 v/v THF/H₂O (0.35 M in substrate) was added LiOH·H₂O (1.5 equiv.). The reaction mixture was stirred at ambient temperature for 16 h and then concentrated under reduced pressure, diluted with ice-cold water and acidified with 1.5 N hydrochloric acid (to pH 3 to 4). The precipitated solid was collected by filtration, washing with diethyl ether and n-pentane, and dried to afford 2-bromobenzo[d]thiazole-6-carboxylic acid as an off-white solid (95% yield).

### Step 2 (Scheme 2): Amide coupling

To a stirred solution of 2-bromobenzo[d]thiazole-6-carboxylic acid (1.0 equiv.) in DMF (0.39 M in substrate) at 0° C. was added n-propylphosphonic acid anhydride cyclic trimer (50% in EtOAc; 2.0 equiv.), DIPEA (4.0 equiv.) and (S)-chroman-4-amine (2.0 equiv.). The reaction mixture was stirred at ambient temperature for 16 h and then concentrated under reduced pressure, diluted with ice-cold water and extracted with EtOAc. The organic extract was washed with brine, dried (Na₂SO₄) and concentrated under reduced pressure to afford the crude product. The latter was processed by flash column chromatography (80-100% EtOAc/petroleum ether) and fractions containing the target material combined and evaporated to afford (S)-2-bromo-N-(chroman-4-yl)benzo[d]thiazole-6-carboxamide as an off-white solid (65% yield).

### Step 3 (Scheme 2): Suzuki coupling

To a stirred, de-gassed mixture of (S)-2-bromo-N-(chroman-4-yl)benzo[d]thiazole-6-carboxamide (1.0 equiv.) in 4:1 v/v1,4-dioxane/H₂O (0.51 M in substrate) under Ar was added (5-formylpyridin-3-yl)boronic acid (1.5 equiv.), Na₂CO₃ (2.0 equiv.) and PdCl₂(dppf). DCM complex (10 mol %). The reaction mixture was heated to reflux under Ar for 16 h and then cooled and filtered through Celite®, washing with EtOAc. The filtrate was washed with water followed by brine and then dried (Na₂SO₄) and evaporated. The crude product was processed by flash column chromatography (40-60% EtOAc/hexanes) and fractions containing the target material combined and evaporated to afford (S)-N-(chroman-4-yl)-2-(5-formylpyridin-3-yl)benzo[d]thiazole-6-carboxamide as an off-white solid (45% yield).

### Step 4 (Scheme 2) leading to Example 62: Aldehyde reduction

To a solution of (S)-N-(chroman-4-yl)-2-(5-formylpyridin-3-yl)benzo[d]thiazole-6-carboxamide (1.0 equiv.) in THF (0.51 M in substrate) at 0° C. was added sodium borohydride (1.5 equiv.) as a single portion. The mixture was stirred at 0° C. for 30 min and then allowed to warm to ambient temperature, stirring for a further period of 1 h (monitored by TLC). The mixture was concentrated under reduced pressure, diluted with EtOAc and the EtOAc mixture washed with water followed by brine. The organic phase was dried (Na₂SO₄) and evaporated to give a crude product that was processed by preparative HPLC to afford (S)-N-(chroman-4-yl)-2-(5-(hydroxymethyl)pyridin-3-yl)benzo[d]thiazole-6-carboxamide as an off-white solid (33% yield).

¹H NMR (free base form): δ_{H} (400 MHz, DMSO-d₆) 9.18 (1H, d, J 2.1), 9.05 (1H, br. d, J 8.1), 8.76 (1H, app. d, J 1.7), 8.71 (1H, d, J 1.9), 8.42-8.41 (1H, m), 8.17 (1H, tented d, J 8.6), 8.11 (1H, tented dd, J 8.6 and 1.7), 7.23 (1H, app. d, J7.6), 7.18 (1H, app. td, J7.7 and 1.5), 6.89 (1H, td, J 7.5 and 1.1), 6.82 (1H, dd, J 8.2 and 1.1), 5.55 (1H, t, J 5.7), 5.35-5.30 (1H, m), 4.68 (2H, d, J 5.7), 4.36-4.23 (2H, m), 2.20-2.03 (2H, m).

### Example 63: (S)-N-(chroman-4-yl)-2-(5-(difluoromethyl)pyridin-3-yl)benzo[d]thiazole-6-carboxamide

### Step 1: Preparation of Boronic Ester

A solution of 3-bromo-5-(difluoromethyl)pyridine (1.0 equiv.), bispinacolatodiboron (3.0 equiv.) and KOAc (3.0 equiv.) in 1,4-dioxane (0.1 M in substrate) was purged with N₂ gas for 15 min. Pd(dppf)Cl₂. DCM (10 mol %) was added and the reaction mixture stirred at 90° C. for 16 h (monitored by TLC). After completion of the reaction, the mixture was cooled, filtered through Celite® and the filtrate evaporated to afford crude (5-(difluoromethyl)pyridin-3-yl)boronic ester as a brown gummy solid that was used directly in the next step.

### Step 2: Suzuki Coupling

To a de-gassed solution of (S)-2-bromo-N-(chroman-4-yl)benzo[d]thiazole-6-carboxamide (1.0 equiv.; prepared as described in Scheme 2) in 9:1 v/v 1,4-dioxane/H₂O (0.1 M in substrate) under N₂ was added (5-(difluoromethyl)pyridin-3-yl)boronic ester (1.5 equiv.), Na₂CO₃ (3.0 equiv.) and Pd(dppf)Cl₂. DCM (10 mol %). The reaction mixture was stirred at 100° C. for 16 h (monitored by TLC). After completion of the reaction, the mixture was cooled and filtered through Celite®. The filtrate was diluted with water and extracted with DCM. The extract was dried (Na₂SO₄) and evaporated to give a crude product that was processed by preparative HPLC to afford (S)-N-(chroman-4-yl)-2-(5-(difluoromethyl)pyridin-3-yl)benzo[d]thiazole-6-carboxamide as an off-white solid.

¹H NMR (free base form): δ_{H} (400 MHz, DMSO-d₆) 9.47-9.45 (1H, m), 9.07 (1H, br. d, J 8.1), 9.01-9.00 (1H, m), 8.78 (1H, dd, J 1.7 and 0.4), 8.68-8.66 (1H, m), 8.21 (1H, tented dd, J 8.6 and 0.4), 8.12 (1H, tented dd, J 8.6 and 1.7), 7.31 (1H, t, ²J_{HF} 55.0), 7.23 (1H, app. d, J 7.6), 7.18 (1H, app. td, J 7.7 and 1.5), 6.89 (1H, td, J 7.5 and 1.1), 6.82 (1H, dd, J 8.2 and 1.1), 5.35-5.30 (1H, m), 4.36-4.23 (2H, m), 2.21-2.04 (2H, m).

### Example 64: (S)-N-(chroman-4-yl)-2-(6-(difluoromethyl)pyridin-3-yl)benzo[d]thiazole-6-carboxamide

Prepared as described in Example 63, using 3-bromo-6-(difluoromethyl)pyridine instead of 3-bromo-5-(difluoromethyl)pyridine.

¹H NMR (free base form): δ_{H} (400 MHz, DMSO-d₆) 9.41 (1H, app. d, J2.2), 9.07 (1H, br. d, J 8.1), 8.78 (1H, dd, J 1.7 and 0.5), 8.69 (1H, dd, J 8.1 and 2.2), 8.21 (1H, dd, J 8.6 and 0.5), 8.12 (1H, dd, J 8.6 and 1.7), 7.92 (1H, d, J 8.1), 7.23 (1H, app. d, J 7.6), 7.18 (1H, app. td, J 7.7 and 1.5), 7.10 (1H, t, ²J_{HF} 54.7), 6.89 (1H, td, J7.5 and 1.1), 6.82 (1H, dd, J 8.2 and 1.1), 5.35-5.30 (1H, m), 4.36-4.23 (2H, m), 2.20-2.04 (2H, m).

### Examples 65 to 67

Examples 65 to 67 may be prepared according to the route shown in Scheme 3.

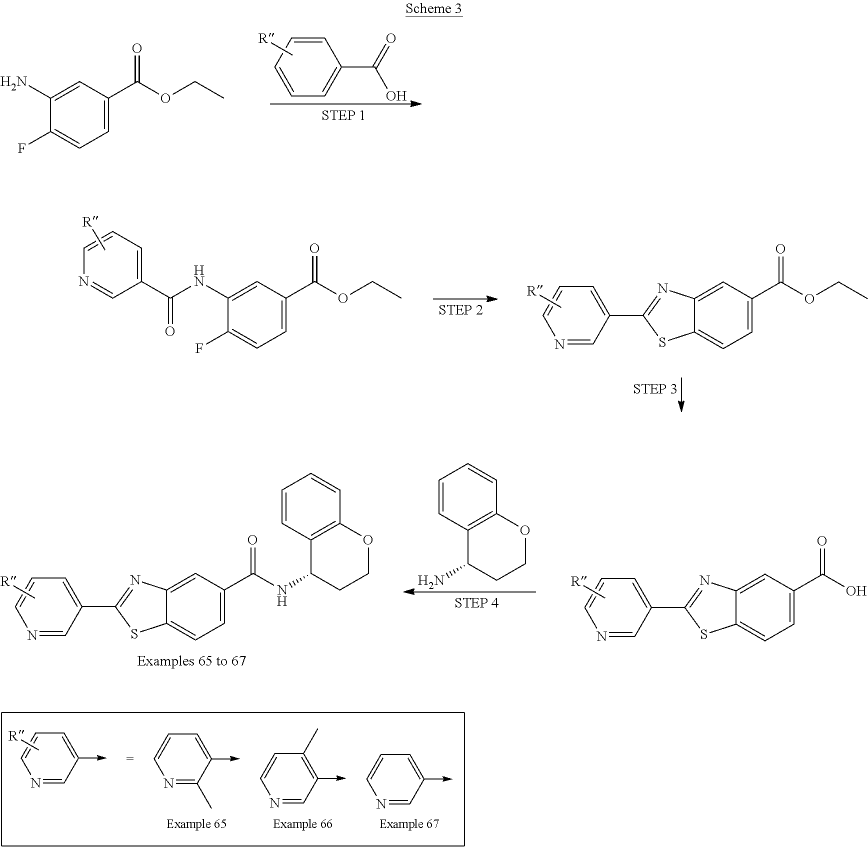

### Step 1 (Scheme 3): Synthesis of ethyl 4-fluoro-3-(nicotinamido)benzoate intermediate

To a stirred solution of nicotinic acid derivative (1.2 equiv.) in DMF (0.1 M in substrate) at 0° C. was added HATU (1.5 equiv.), DIPEA (2.0 equiv.) and ethyl 3-amino-4-fluorobenzoate (1.0 equiv.). The reaction mixture was allowed to stir at ambient temperature for 16 h and then concentrated in vacuo. The residue was diluted with DCM and the resulting solution washed with water followed by brine. The organic layer was dried (Na₂SO₄) and evaporated to afford the nicotinamide derivative, which was used in the next step without further purification.

### Step 2 (Scheme 3): Synthesis of ethyl 2-(pyridin-3-yl)benzo[d]thiazole-5-carboxylate intermediate

To a solution of the ethyl 4-fluoro-3-(nicotinamido)benzoate derivative from Step 1 (1.0 equiv.) in toluene (0.1 M in substrate) was added Lawesson's Reagent (1.5 equiv.). The reaction mixture was stirred under reflux for 24 h (monitored by LCMS). After consumption of starting material the mixture was concentrated in vacuo. The residue was diluted with DCM and the resulting solution washed with water followed by brine. The organic layer was dried (Na₂SO₄) and evaporated to give a residue that was processed by flash chromatography (MeOH/DCM eluent system). Fractions containing the product were combined and evaporated to give a solid that was triturated with diethyl ether and dried to afford the ethyl 2-(pyridin-3-yl)benzo[d]thiazole-5-carboxylate derivative as an off-white solid.

### Step 3 (Scheme 3): Synthesis of 2-bromobenzo[d]thiazole-5-carboxylic acid intermediate

To a solution of the ethyl 2-(pyridin-3-yl)benzo[d]thiazole-5-carboxylate derivative from Step 2 (1.0 equiv.) in 3:1:1 v/v/v THF/MeOH/H₂O (0.1 M in substrate) was added LiOH·H₂O (2.0 equiv.). The reaction mixture was stirred at ambient temperature for 3 h and then concentrated in vacuo. The residue was diluted with ice-cold water and the resulting solution acidified with citric acid (to pH 4 to 5) and then extracted with DCM. The extract was dried (Na₂SO₄) and evaporated to afford the 2-(pyridin-3-yl)benzo[d]thiazole-5-carboxylic acid derivative as an off-white solid; this was used in the next step without further purification.

### Step 4 (Scheme 3) leading to Examples 65 to 67: General amide coupling procedure

To a solution of 2-(pyridin-3-yl)benzo[d]thiazole-5-carboxylic acid derivative from Step 3 (1.0 equiv.) in DMF (0.1 M in substrate) at 0° C. was added n-propylphosphonic acid anhydride cyclic trimer (50% in EtOAc; 2.0 equiv.), DIPEA (4.0 equiv.) and (S)-chroman-4-amine (2.0 equiv.). The reaction mixture was brought to ambient temperature, stirring for 16 h, and then concentrated in vacuo. The residue was diluted with ice-cold water and extracted with DCM.

The extract was washed with brine, dried (Na₂SO₄) and evaporated to give a residue that was processed by preparative HPLC to afford the product as an off-white solid.

### Example 65: (S)-N-(chroman-4-yl)-2-(2-methylpyridin-3-yl)benzo[d]thiazole-5-carboxamide

Prepared using 2-methylnicotinic acid in Step 1 of Scheme 3.

¹H NMR (free base form): δ_{H} (400 MHz, DMSO-d₆) 9.11 (1H, br. d, J8.2), 8.70 (1H, app. d, J 1.6), 8.64 (1H, dd, J4.8 and 1.6), 8.30 (1H, tented d, J8.4), 8.23 (1H, dd, J7.9 and 1.6), 8.06 (1H, tented dd, J 8.4 and 1.6), 7.47 (1H, dd, J7.9 and 4.8), 7.23 (1H, app. d, J7.6), 7.17 (1H, app. td, J7.7 and 1.5), 6.89 (1H, td, J7.5 and 1.1), 6.82 (1H, dd, J 8.2 and 1.1), 5.36-5.31 (1H, m), 4.37-4.23 (2H, m), 2.8 (3H, s), 2.21-2.05 (2H, m).

### Example 66: (S)-N-(chroman-4-yl)-2-(4-methylpyridin-3-yl)benzo[d]thiazole-5-carboxamide

Prepared using 4-methylnicotinic acid in Step 1 of Scheme 3.

¹H NMR (free base form): δ_{H} (400 MHz, DMSO-d₆) 9.12 (1H, br. d, J8.1), 8.99 (1H, s), 8.70 (1H, app. d, J 1.6), 8.60 (1H, d, J 5.0), 8.31 (1H, tented d, J 8.4), 8.07 (1H, tented dd, J 8.4 and 1.6), 7.50 (1H, app. d, J 5.0), 7.23 (1H, app. d, J 7.6), 7.17 (1H, app. td, J 7.7 and 1.5), 6.89 (1H, td, J7.5 and 1.1), 6.82 (1H, dd, J 8.2 and 1.1), 5.36-5.31 (1H, m), 4.37-4.23 (2H, m), 2.68 (3H, s), 2.21-2.05 (2H, m).

### Example 67: (S)-N-(chroman-4-yl)-2-(pyridin-3-yl)benzo[d]thiazole-5-carboxamide

Prepared using nicotinic acid in Step 1 of Scheme 3.

¹H NMR (free base form): δ_{H} (400 MHz, DMSO-d₆) 9.29 (1H, dd, J 2.3 and 0.7), 9.09 (1H, br. d, J 8.1), 8.78 (1H, dd, J 4.8 and 1.6), 8.68 (H, dd, J 1.7 and 0.4), 8.47 (1H, ddd, J 8.0, 2.3 and 1.6), 8.29 (1H, tented dd, J 8.4 and 0.4), 8.05 (1H, tented dd, J 8.4 and 1.7), 7.64 (1H, ddd, J 8.0, 4.8 and 0.7), 7.23 (1H, app. d, J 7.6), 7.18 (1H, app. td, J 7.7 and 1.5), 6.89 (1H, td, J7.5 and 1.1), 6.82 (1H, dd, J 8.2 and 1.1), 5.36-5.31 (1H, m), 4.37-4.23 (2H, m), 2.21-2.05 (2H, m).

### Examples 68 to 79

Examples 68 to 79 may be prepared according to the route shown in Scheme 4.

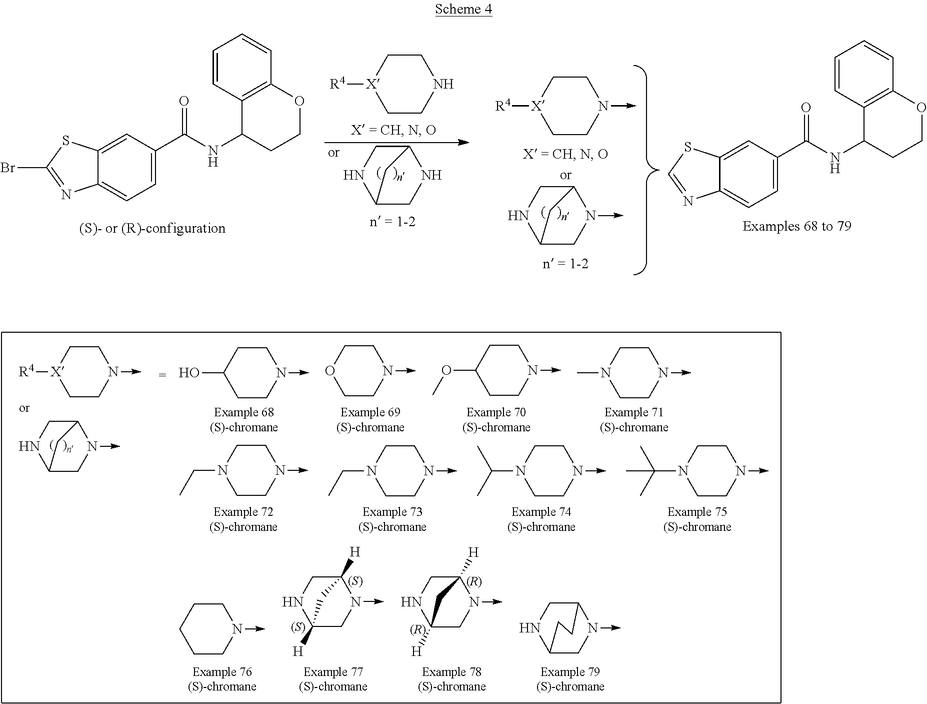

Preparation of Examples 68 to 79 by Buchwald-Hartwig coupling (Scheme 4)

To a de-gassed solution of (S)- or (R)-2-bromo-N-(chroman-4-yl)benzo[d]thiazole-6-carboxamide [1.0 equiv.; prepared as in Scheme 2 from 2-bromobenzo[d]thiazole-6-carboxylic acid with (S)- or (R)-chroman-4-amine] in toluene (0.067 M in substrate) under Ar was added the amine derivative (1.5 equiv.), Cs₂CO₃ (2.0 equiv.), (rac)-2,2′-bis(diphenylphosphino)-1,1′-binaphthyl (20 mol %) and PdOAc₂ (10 mol %). The reaction mixture was stirred at 110° C. for 16 h (monitored by LCMS). The mixture was then cooled to ambient temperature, concentrated in vacuo and filtered through Celite®, washing with 10% MeOH/DCM. The filtrate was evaporated to give a residue that was processed by flash column chromatography (50-100% EtOAc/petroleum ether) to afford the desired product.

### Example 68: (S)-N-(chroman-4-yl)-2-(4-hydroxypiperidin-1-yl)benzo[d]thiazole-6-carboxamide

Prepared using (S)-2-bromo-N-(chroman-4-yl)benzo[d]thiazole-6-carboxamide and 4-hydroxypiperidine as the amine component.

¹H NMR (free base form): δ_{H} (400 MHz, DMSO-d₆) 8.75 (1H, br. d, J 8.2), 8.32 (1H, app. d, J 1.7), 7.85 (1H, tented dd, J 8.5 and 1.7), 7.43 (1H, tented d, J 8.5), 7.20-7.12 (2H, m), 6.87 (1H, td, J7.5 and 1.1), 6.80 (1H, dd, J8.2 and 1.1), 5.31-5.26 (1H, m), 4.94 (1H, br. s), 4.34-4.20 (2H, m), 3.90-3.82 (2H, m), 3.82-4.74 (1H, m), 3.43-3.35 (2H, m), 2.16-2.00 (2H, m), 1.89-1.81 (2H, m), 1.53-1.42 (2H, m).

### Example 69: (S)-N-(chroman-4-yl)-2-morpholinobenzo[d]thiazole-6-carboxamide

Prepared using (S)-2-bromo-N-(chroman-4-yl)benzo[d]thiazole-6-carboxamide and morpholine as the amine component.

¹H NMR (free base form): δ_{H} (400 MHz, DMSO-d₆) 8.78 (1H, br. d, J 8.2), 8.36 (1H, app. d, J 1.7), 7.88 (1H, tented dd, J 8.5 and 1.7), 7.48 (1H, tented d, J 8.5), 7.21-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.75-3.71 (4H, m), 3.61-3.57 (4H, m), 2.16-2.00 (2H, m).

### Example 70: (S)-N-(chroman-4-yl)-2-(4-methoxypiperidin-1-yl)benzo[d]thiazole-6-carboxamide

Prepared using (S)-2-bromo-N-(chroman-4-yl)benzo[d]thiazole-6-carboxamide and 4-methoxypiperidine as the amine component.

¹H NMR (free base form): δ_{H} (400 MHz, DMSO-d₆) 8.75 (1H, br. d, J 8.2), 8.32 (1H, app. d, J 1.7), 7.86 (1H, tented dd, J 8.5 and 1.7), 7.44 (1H, tented d, J 8.5), 7.19-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.84-3.77 (2H, m), 3.52-3.40 (3H, m), 3.29 (3H, s), 2.16-2.00 (2H, m), 1.99-1.90 (2H, m), 1.61-1.51 (2H, m).

### Example 71: (S)-N-(chroman-4-yl)-2-(4-methylpiperazin-1-yl)benzo[d]thiazole-6-carboxamide

Prepared using (S)-2-bromo-N-(chroman-4-yl)benzo[d]thiazole-6-carboxamide and 1-methylpiperazine as the amine component.

¹H NMR (free base form): δ_{H} (400 MHz, DMSO-d₆) 8.76 (1H, br. d, J8.1), 8.34 (1H, app. d, J 1.6), 7.86 (1H, tented dd, J 8.5 and 1.6), 7.46 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.64-3.56 (4H, m), 2.48-2.42 (4H, m), 2.24 (3H, s), 2.16-2.00 (2H, m).

### Example 72: (S)-N-(chroman-4-yl)-2-(4-ethylpiperazin-1-yl)benzo[d]thiazole-6-carboxamide

Prepared using (S)-2-bromo-N-(chroman-4-yl)benzo[d]thiazole-6-carboxamide and 1-ethylpiperazine as the amine component.

¹H NMR (free base form): δ_{H} (400 MHz, DMSO-d₆) 8.76 (1H, br. d, J 8.2), 8.34 (1H, app. d, J 1.7), 7.86 (1H, tented dd, J 8.5 and 1.7), 7.46 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.62-3.56 (4H, m), 2.53-2.49 (4H, m), 2.39 (2H, q J 7.1), 2.16-2.00 (2H, m), 1.03 (3H, t, J 7.1).

### Example 73: (R)-N-(chroman-4-yl)-2-(4-ethylpiperazin-1-yl)benzo[d]thiazole-6-carboxamide

Prepared using (R)-2-bromo-N-(chroman-4-yl)benzo[d]thiazole-6-carboxamide and 1-ethylpiperazine as the amine component.

¹H NMR (free base form): δ_{H} (400 MHz, DMSO-d₆) 8.76 (1H, br. d, J 8.2), 8.34 (1H, app. d, J 1.7), 7.86 (1H, tented dd, J 8.5 and 1.7), 7.46 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.62-3.56 (4H, m), 2.53-2.49 (4H, m), 2.39 (2H, q J 7.1), 2.16-2.00 (2H, m), 1.03 (3H, t, J 7.1).

### Example 74: (S)-N-(chroman-4-yl)-2-(4-isopropylpiperazin-1-yl)benzo[d]thiazole-6-carboxamide

Prepared using (S)-2-bromo-N-(chroman-4-yl)benzo[d]thiazole-6-carboxamide and 1-isopropylpiperazine as the amine component.

¹H NMR (free base form): δ_{H} (400 MHz, DMSO-d₆) 8.75 (1H, br. d, J 8.2), 8.33 (1H, app. d, J 1.6), 7.86 (1H, tented dd, J 8.5 and 1.6), 7.45 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.60-3.55 (4H, m), 2.73 (1H, septet, J 6.6), 2.59-2.54 (4H, m), 2.16-2.00 (2H, m), 0.99 (6H, d, J6.6).

### Example 75: (S)-2-(4-(tert-butyl)piperazin-1-yl)-N-(chroman-4-yl)benzo[d]thiazole-6-carboxamide

Prepared using (S)-2-bromo-N-(chroman-4-yl)benzo[d]thiazole-6-carboxamide and 1-(tert-butyl)piperazine as the amine component.

¹H NMR (free base form): δ_{H} (400 MHz, DMSO-d₆) 8.76 (1H, br. d, J 8.2), 8.33 (1H, app. d, J 1.6), 7.86 (1H, tented dd, J 8.5 and 1.6), 7.45 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.59-3.53 (4H, m), 2.65-2.59 (4H, m), 2.16-2.00 (2H, m), 1.04 (9H, s).

### Example 76: (S)-N-(chroman-4-yl)-2-(piperidin-1-yl)benzo[d]thiazole-6-carboxamide

Prepared using (S)-2-bromo-N-(chroman-4-yl)benzo[d]thiazole-6-carboxamide and piperidine as the amine component.

¹H NMR (free base form): δ_{H} (400 MHz, DMSO-d₆) 8.74 (1H, br. d, J 8.2), 8.31 (1H, app. d, J 1.7), 7.85 (1H, tented dd, J 8.5 and 1.7), 7.42 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.62-3.56 (4H, m), 2.16-2.00 (2H, m), 1.69-1.55 (6H, m).

### Example 77: 2-((1 S,4S)-2,5-diazabicyclo[2.2.1]heptan-2-yl)-N-((S)-chroman-4-yl)benzo[d]thiazole-6-carboxamide

Prepared using (S)-2-bromo-N-(chroman-4-yl)benzo[d]thiazole-6-carboxamide and (1 S,4S)-2,5-diazabicyclo[2.2.1]heptane as the amine component.

¹H NMR (free base form): δ_{H} (400 MHz, DMSO-d₆) 8.73 (1H, br. d, J8.1), 8.31 (1H, app. d, J 1.7), 7.85 (1H, tented dd, J 8.5 and 1.7), 7.43 (1H, tented d, J 8.5), 7.20-7.12 (2H, m), 6.87 (1H, td, J7.5 and 1.1), 6.80 (1H, dd, J8.2 and 1.1), 5.31-5.26 (1H, m), 4.52 (1H, br. s), 4.34-4.20 (2H, m), 3.71 (1H, app. s), 3.57 (1H, dd, J 9.1 and 1.6), 2.96-2.89 (2H, m), 2.57-2.44 (2H, m), 2.16-2.00 (2H, m), 1.88 (1H, app. d, J9.4), 1.73 (1H, app. d, J9.4).

### Example 78: 2-((1R,4R)-2,5-diazabicyclo[2.2.1]heptan-2-yl)-N-((S)-chroman-4-yl)benzo[d]thiazole-6-carboxamide

Prepared using (S)-2-bromo-N-(chroman-4-yl)benzo[d]thiazole-6-carboxamide and (1R,4R)-2,5-diazabicyclo[2.2.1]heptane as the amine component.

¹H NMR (free base form): δ_{H} (400 MHz, CD₃OD) 8.20 (1H, app. d, J 1.8), 7.83 (1H, tented dd, J 8.5 and 1.8), 7.47 (1H, tented d, J8.5), 7.23 (1H, app. d, J7.6), 7.15 (1H, app. td, J7.7 and 1.5), 6.89 (1H, td, J7.5 and 1.1), 6.80 (1H, dd, J 8.2 and 1.1), 5.35 (1H, t, J6.1), 4.69-4.58 (1H, m), 4.34-4.24 (2H, m), 3.89 (1H, app. s), 3.69 (1H, dd, J 9.6 and 2.0), 3.50-3.43 (1H, m), 3.11-3.04 (2H, m), 2.27-2.11 (2H, m), 2.05 (1H, app. d, J 10.2), 1.89 (1H, app. d, J 10.2). [Amide NH and amine NH lost to deuterium exchange.]

### Example 79: 2-(2,5-diazabicyclo[2.2.2]octan-2-yl)-N-((S)-chroman-4-yl)benzo[d]thiazole-6-carboxamide

Prepared using (S)-2-bromo-N-(chroman-4-yl)benzo[d]thiazole-6-carboxamide and 2,5-diazabicyclo[2.2.2]octane as the amine component.

¹H NMR (free base form): δ_{H} (400 MHz, DMSO-d₆) 8.73 (1H, br. d, J 8.2), 8.32 (1H, app. d, J 1.7), 7.85 (1H, tented dd, J 8.5 and 1.7), 7.42 (1H, tented d, J 8.5), 7.20-7.12 (2H, m), 6.87 (1H, td, J 7.5 and 1.1), 6.80 (1H, dd, J 8.2 and 1.1), 5.31-5.26 (1H, m), 4.34-4.20 (2H, m), 3.70-3.51 (2H, m), 3.21-3.14 (1H, m), 3.11-3.06 (1H, m), 3.06-2.98 (1H, m), 2.64-2.46 (1H, m), 2.16-2.00 (2H, m), 2.00-1.69 (5H, m).

### Examples 80 to 82

Examples 80 to 82 may be prepared according to the route shown in Scheme 5.

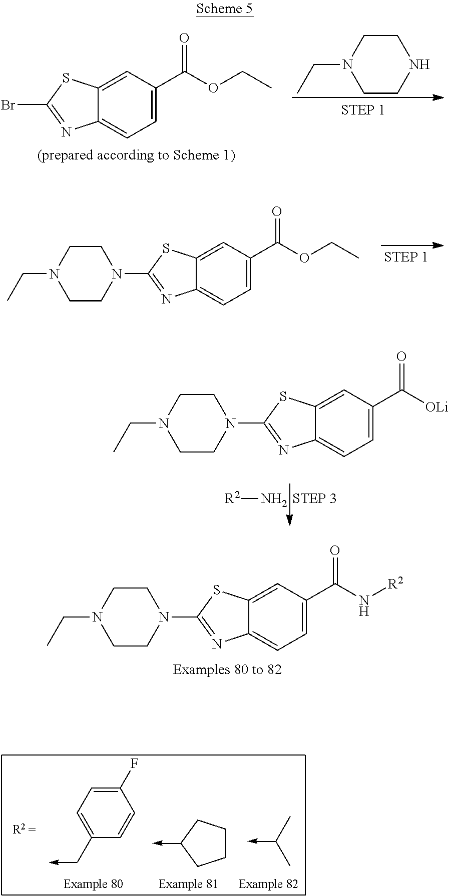

### Step 1 (Scheme 5): Synthesis of ethyl 2-(4-ethylpiperazin-1-yl)benzo[d]thiazole-6-carboxylate

To a stirred solution of ethyl 2-bromobenzo[d]thiazole-6-carboxylate (1.0 equiv.; prepared as in Scheme 1) in acetonitrile (0.07 M in substrate) was added K₂CO₃ (3.0 equiv.) and 1-ethylpiperazine (2.0 equiv.). The reaction mixture was heated at 80° C. for 16 h (monitored by TLC) and then concentrated in vacuo. The residue was diluted with water and the resulting mixture extracted with DCM. The extract was washed with brine, dried (Na₂SO₄) and evaporated to give a residue that was triturated with petroleum ether and dried to afford ethyl 2-(4-ethylpiperazin-1-yl)benzo[d]thiazole-6-carboxylate as an orange solid (81% yield).

### Step 2 (Scheme 5): Synthesis of 2-(4-ethylpiperazin-1-yl)benzo[d]thiazole-6-carboxylic acid lithium salt

To a mixture of ethyl 2-(4-ethylpiperazin-1-yl)benzo[d]thiazole-6-carboxylate (1.0 equiv.) in 1.9:1:1 v/v/v THF/MeOH/H₂O (0.091 M in substrate) was added LiOH·H₂O (1.0 equiv.). The reaction mixture was stirred at ambient temperature for 16 h and then evaporated to dryness under reduced pressure. The residue was triturated with petroleum ether and dried to afford 2-(4-ethylpiperazin-1-yl)benzo[d]thiazole-6-carboxylic acid lithium salt as an orange solid (96% yield).

### Step 3 (Scheme 5) leading to Examples 80 to 82: General procedure for amide coupling

To a mixture of 2-(4-ethylpiperazin-1-yl)benzo[d]thiazole-6-carboxylic acid lithium salt (1.0 equiv.) in DCM (0.1 M in substrate) at 0° C. was added n-propylphosphonic acid anhydride cyclic trimer (50% in EtOAc; 2.0 equiv.) and DIPEA (4.0 equiv.). The mixture was stirred at 0° C. for 15 min prior to addition of the amine component (1.2 equiv.). The reaction mixture was brought to ambient temperature, stirring for 16 h, and then diluted with DCM and washed with water followed by brine. The organic phase was dried (Na₂SO₄) and evaporated to afford a residue that was processed by preparative HPLC to afford the target amide.

### Example 80: 2-(4-ethylpiperazin-1-yl)-N-(4-fluorobenzyl)benzo[d]thiazole-6-carboxamide

Prepared using 4-fluorobenzylamine as the amine component.

¹H NMR (free base form): δ_{H} (400 MHz, CD₃OD) 8.19 (1H, dd, J 1.8 and 0.4), 7.81 (1H, tented dd, J 8.5 and 1.8), 7.50 (1H, tented dd, J 8.5 and 0.4), 7.40-7.35 (2H, m), 7.08-7.02 (2H, m), 4.55 (2H, s), 3.72-3.67 (4H, m), 2.65-2.61 (4H, m), 2.52 (2H, q, J 7.2), 1.15 (3H, t, J 7.2). [Amide NH lost to deuterium exchange.]

### Example 81: N-cyclopentyl-2-(4-ethylpiperazin-1-yl)benzo[d]thiazole-6-carboxamide

Prepared using cyclopentylamine as the amine component.

¹H NMR (free base form): δ_{H} (400 MHz, CD₃OD) 8.15 (1H, app. d, J 1.8), 7.77 (1H, tented dd, J 8.5 and 1.8), 7.48 (1H, tented d, J 8.5), 4.36-4.29 (1H, m), 3.72-3.67 (4H, m), 2.66-2.61 (4H, m), 2.52 (2H, q, J 7.2), 2.08-1.98 (2H, m), 1.85-1.72 (2H, m), 1.72-1.54 (4H, m), 1.15 (3H, t, J7.2). [Amide NH lost to deuterium exchange.]

### Example 82: 2-(4-ethylpiperazin-1-yl)-N-isopropylbenzo[d]thiazole-6-carboxamide

Prepared using isopropylamine as the amine component.

¹H NMR (free base form): δ_{H} (400 MHz, DMSO-d₆) 8.25 (1H, app. d, J 1.8), 8.12 (1H, d, J7.7), 7.78 (1H, tented dd, J8.6 and 1.8), 7.44 (1H, tented d, J 8.5), 4.15-4.03 (1H, m), 3.61-3.56 (4H, m), 2.52-2.47 (4H, m), 2.39 (2H, q, J7.2), 1.16 (6H, d, J6.1), 1.03 (3H, t, J7.2).

### Examples 83 to 95

Examples 83 to 95 may be prepared according to the route shown in Scheme 6.

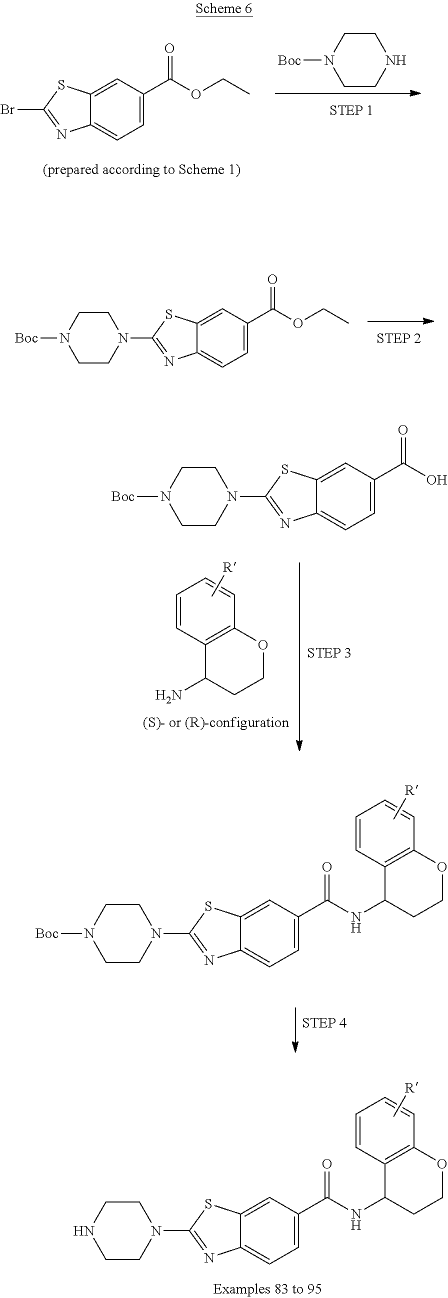

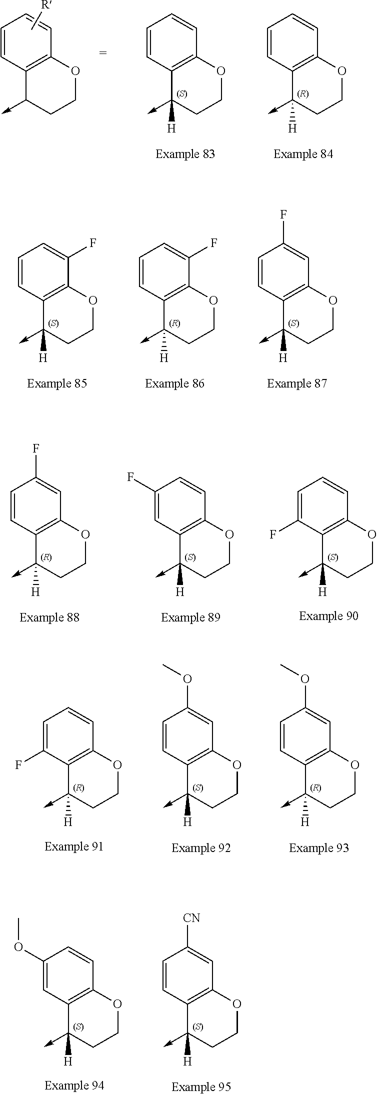

### Step 1 (Scheme 6): Synthesis of ethyl 2-(4-(tert-butoxycarbonyl)piperazin-1-yl)benzo[d]thiazole-6-carboxylate

To a de-gassed mixture of ethyl 2-bromobenzo[d]thiazole-6-carboxylate (1.0 equiv.; prepared as in Scheme 1) in toluene (0.12 M in substrate) under Ar was added tert-butyl piperazine-1-carboxylate (1.5 equiv.), Cs₂CO₃ (2.0 equiv.), (rac)-2,2′-bis(diphenylphosphino)-1,1′-binaphthyl (30 mol %) and PdOAc₂ (10 mol %). The reaction mixture was stirred at 100° C. for 16 h (monitored by LCMS). The mixture was then cooled to ambient temperature, concentrated in vacuo and the residue diluted with water and extracted with DCM. The extract was washed with brine, dried (Na₂SO₄) and evaporated to give a residue that was processed by flash chromatography (30-35% EtOAc/hexanes eluent). Fractions containing the product were combined and evaporated to afford ethyl 2-(4-(tert-butoxycarbonyl)piperazin-1-yl)benzo[d]thiazole-6-carboxylate as a brown solid (73% yield).

### Step 2 (Scheme 6): Synthesis of 2-(4-(tert-butoxycarbonyl)piperazin-1-yl)benzo[d]thiazole-6-carboxylic acid

To a mixture of ethyl 2-(4-(tert-butoxycarbonyl)piperazin-1-yl)benzo[d]thiazole-6-carboxylate (1.0 equiv.) in 2:2:1 v/v/v THF (0.21 M in substrate) was added LiOH·H₂O (2.0 equiv.). The reaction mixture was stirred at ambient temperature for 16 h (monitored by TLC) and then evaporated. The residue was diluted with ice-cold water and acidified with 1.5 N hydrochloric acid to precipitate a solid that was collected by filtration, washing with water, and dried in vacuo to afford 2-(4-(tert-butoxycarbonyl)piperazin-1-yl)benzo[d]thiazole-6-carboxylic acid as an off-white solid (99% yield).

### Step 3 (Scheme 6): Synthesis of tert-butyl (R)/(S)-4-(6-(chroman-4-ylcarbamoyl)benzo[d]thiazol-2-yl)piperazine-1-carboxylate derivatives

To a solution of 2-(4-(tert-butoxycarbonyl)piperazin-1-yl)benzo[d]thiazole-6-carboxylic acid (1.0 equiv.) in DCM (0.05 M in substrate) at 0° C. was added n-propylphosphonic acid anhydride cyclic trimer (50% in EtOAc; 2.0 equiv.) and DIPEA (4.0 equiv.). The mixture was stirred at 0° C. for 15 min prior to addition of the (R)- or (S)-chroman-4-amine component (1.2 equiv.; prepared according to General Procedure 1). The reaction mixture was brought to ambient temperature, stirring for 16 h, and then diluted with DCM and washed with water followed by brine. The organic phase was dried (Na₂SO₄) and evaporated to afford a residue that was processed by preparative HPLC to afford the tert-butyl (R)/(S)-4-(6-(chroman-4-ylcarbamoyl)benzo[d]thiazol-2-yl)piperazine-1-carboxylate derivative as an off-white solid.

### Step 4 (Scheme 6): Synthesis of (R)/(S)-N-(chroman-4-yl)-2-(piperazin-1-yl)benzo[d]thiazole-6-carboxamide derivatives

To a stirred solution of the tert-butyl (R)/(S)-4-[6-(chroman-4-ylcarbamoyl)benzo[d]thiazol-2-yl]piperazine-1-carboxylate (1.0 equiv.) in DCM (0.1 M in substrate) at 0° C. was added 2 N HCl in diethyl ether (5.0 equiv. HCl). The reaction mixture was stirred at ambient temperature for 4 h (monitored by TLC). After consumption of starting material, the mixture was evaporated and the resulting residue triturated with hexanes and then lyophilized to afford the hydrochloride salt of the (R)/(S)-N-(chroman-4-yl)-2-(piperazin-1-yl)benzo[d]thiazole-6-carboxamide derivative as an off-white solid. The hydrochloride salt may optionally be desalted by partition between aqueous base and organic solvent, with the organic phase dried (Na₂SO₄) and evaporated to yield the free base form.

### Example 83: (S)-N-(chroman-4-yl)-2-(piperazin-1-yl)benzo[d]thiazole-6-carboxamide

Prepared using (S)-chroman-4-amine in Step 3 of Scheme 6.

¹H NMR (hydrochloride salt form): δ_{H} (400 MHz, DMSO-d₆) 9.32 (2H, br. s), 8.80 (1H, br. d, J 8.2), 8.40 (1H, app. d, J 1.7), 7.90 (1H, tented dd, J 8.5 and 1.7), 7.52 (1H, tented d, J 8.5), 7.20-7.13 (2H, m), 6.87 (1H, td, J7.5 and 1.1), 6.80 (1H, dd, J 8.2 and 1.1), 5.31-5.26 (1H, m), 4.34-4.20 (2H, m), 3.88-3.82 (4H, m), 3.30-3.23 (4H, m), 2.16-2.00 (2H, m).

### Example 84: (R)-N-(chroman-4-yl)-2-(piperazin-1-yl)benzo[d]thiazole-6-carboxamide

Prepared using (R)-chroman-4-amine in Step 3 of Scheme 6.

¹H NMR (free base form): δ_{H} (400 MHz, DMSO-d₆) 8.75 (1H, br. d, J8.1), 8.32 (1H, app. d, J 1.7), 7.85 (1H, tented dd, J 8.5 and 1.7), 7.44 (1H, tented d, J 8.5), 7.20-7.12 (2H, m), 6.86 (1H, td, J 7.5 and 1.1), 6.79 (1H, dd, J 8.2 and 1.1), 5.30-5.25 (1H, m), 4.34-4.20 (2H, m), 3.54-3.49 (4H, m), 2.83-2.78 (4H, m), 2.16-2.00 (2H, m).

### Example 85: (S)-N-(8-fluorochroman-4-yl)-2-(piperazin-1-yl)benzo[d]thiazole-6-carboxamide

Prepared using (S)-8-fluorochroman-4-amine in Step 3 of Scheme 6.

¹H NMR (hydrochloride salt form): δ_{H} (400 MHz, DMSO-d₆) 9.36 (2H, br. s), 8.85 (1H, br. d, J 8.2), 8.39 (1H, app. d, J 1.7), 7.89 (1H, tented dd, J 8.5 and 1.7), 7.52 (1H, tented d, J 8.5), 7.14-7.06 (1H, m), 7.03-6.97 (1H, m), 6.88-6.80 (1H, m), 5.34-5.28 (1H, m), 4.43-4.29 (2H, m), 3.88-3.81 (4H, m), 3.30-3.21 (4H, m), 2.21-2.04 (2H, m).

### Example 86: (R)-N-(8-fluorochroman-4-yl)-2-(piperazin-1-yl)benzo[d]thiazole-6-carboxamide

Prepared using (R)-8-fluorochroman-4-amine in Step 3 of Scheme 6.

¹H NMR (hydrochloride salt form): δ_{H} (400 MHz, DMSO-d₆) 9.35 (2H, br. s), 8.84 (1H, br. d, J 8.1), 8.39 (1H, app. d, J1.7), 7.89 (1H, tented dd, J 8.5 and 1.7), 7.52 (1H, tented d, J 8.5), 7.14-7.06 (1H, m), 7.03-6.97 (1H, m), 6.88-6.80 (1H, m), 5.34-5.28 (1H, m), 4.43-4.29 (2H, m), 3.88-3.81 (4H, m), 3.30-3.21 (4H, m), 2.21-2.04 (2H, m).

### Example 87: (S)-N-(7-fluorochroman-4-yl)-2-(piperazin-1-yl)benzo[d]thiazole-6-carboxamide

Prepared using (S)-7-fluorochroman-4-amine in Step 3 of Scheme 6.

¹H NMR (hydrochloride salt form): δ_{H} (400 MHz, DMSO-d₆) 9.32 (2H, br. s), 8.77 (1H, br. d, J 8.1), 8.39 (1H, app. d, J1.7), 7.89 (1H, tented dd, J 8.5 and 1.7), 7.52 (1H, tented d, J 8.5), 7.21 (1H, dd, J 8.5 and 6.9), 6.72 (1H, td, J 8.5 and 2.6), 6.67 (1H, dd, J 10.6 and 2.6), 5.27-5.22 (1H, m), 4.35-4.24 (2H, m), 3.87-3.81 (4H, m), 3.31-3.22 (4H, m), 2.16-2.00 (2H, m).

### Example 88: (R)-N-(7-fluorochroman-4-yl)-2-(piperazin-1-yl)benzo[d]thiazole-6-carboxamide

Prepared using (R)-7-fluorochroman-4-amine in Step 3 of Scheme 6.

¹H NMR (hydrochloride salt form): δ_{H} (400 MHz, DMSO-d₆) 9.31 (2H, br. s), 8.78 (1H, br. d, J 8.1), 8.39 (1H, app. d, J1.7), 7.89 (1H, tented dd, J 8.5 and 1.7), 7.52 (1H, tented d, J 8.5), 7.21 (1H, dd, J 8.5 and 6.9), 6.72 (1H, td, J 8.5 and 2.6), 6.67 (1H, dd, J 10.6 and 2.6), 5.27-5.22 (1H, m), 4.35-4.24 (2H, m), 3.88-3.81 (4H, m), 3.30-3.22 (4H, m), 2.16-2.00 (2H, m).

### Example 89: (S)-N-(6-fluorochroman-4-yl)-2-(piperazin-1-yl)benzo[d]thiazole-6-carboxamide

Prepared using (S)-6-fluorochroman-4-amine in Step 3 of Scheme 6.

¹H NMR (hydrochloride salt form): δ_{H} (400 MHz, DMSO-d₆) 9.28 (2H, br. s), 8.83 (1H, br. d, J 8.1), 8.40 (1H, app. d, J1.7), 7.90 (1H, tented dd, J 8.5 and 1.7), 7.53 (1H, tented d, J 8.5), 7.06-6.95 (2H, m), 6.83 (1H, dd, J 8.9 and 4.9), 5.29-5.24 (1H, m), 4.33-4.19 (2H, m), 3.87-3.81 (4H, m), 3.30-3.23 (4H, m), 2.16-2.00 (2H, m).

### Example 90: (S)-N-(5-fluorochroman-4-yl)-2-(piperazin-1-yl)benzo[d]thiazole-6-carboxamide

Prepared using (S)-5-fluorochroman-4-amine in Step 3 of Scheme 6.

¹H NMR (free base form): δ_{H} (400 MHz, DMSO-d₆) 8.84 (1H, br. d, J7.3), 8.30 (1H, app. d, J 1.7), 7.83 (1H, tented dd, J 8.5 and 1.7), 7.42 (1H, tented d, J 8.5), 7.23 (1H, td, J 8.2 and 6.9), 6.75-6.69 (2H, m), 5.35-5.29 (1H, m), 4.34-4.20 (2H, m), 3.53-3.48 (4H, m), 2.82-2.77 (4H, m), 2.09-1.93 (2H, m).

### Example 91: (R)-N-(5-fluorochroman-4-yl)-2-(piperazin-1-yl)benzo[d]thiazole-6-carboxamide

Prepared using (R)-5-fluorochroman-4-amine in Step 3 of Scheme 6.

¹H NMR (free base form): δ_{H} (400 MHz, DMSO-d₆) 8.81 (1H, br. d, J7.4), 8.30 (1H, app. d, J 1.7), 7.83 (1H, tented dd, J 8.5 and 1.7), 7.42 (1H, tented d, J 8.5), 7.23 (1H, td, J 8.2 and 6.9), 6.74-6.68 (2H, m), 5.35-5.29 (1H, m), 4.34-4.20 (2H, m), 3.53-3.48 (4H, m), 2.82-2.77 (4H, m), 2.09-1.93 (2H, m).

### Example 92: (S)-N-(7-methoxychroman-4-yl)-2-(piperazin-1-yl)benzo[d]thiazole-6-carboxamide

Prepared using (S)-7-methoxychroman-4-amine in Step 3 of Scheme 6.

¹H NMR (free base form): δ_{H} (400 MHz, DMSO-d₆) 8.65 (1H, br. d, J8.1), 8.32 (1H, app. d, J 1.7), 7.85 (1H, tented dd, J 8.5 and 1.7), 7.43 (1H, tented d, J 8.5), 7.07 (1H, d, J 8.5), 6.49 (1H, dd, J8.5 and 2.2), 6.36 (1H, d, J2.2), 5.22-5.17 (1H, m), 4.32-4.17 (2H, m), 3.70 (3H, s), 3.55-3.49 (4H, m), 2.85-2.78 (4H, m), 2.13-1.95 (2H, m).

### Example 93: (R)-N-(7-methoxychroman-4-yl)-2-(piperazin-1-yl)benzo[d]thiazole-6-carboxamide

Prepared using (R)-7-methoxychroman-4-amine in Step 3 of Scheme 6.

¹H NMR (free base form): δ_{H} (400 MHz, DMSO-d₆) 8.66 (1H, br. d, J 8.2), 8.31 (1H, app. d, J 1.7), 7.84 (1H, tented dd, J 8.5 and 1.7), 7.43 (1H, tented d, J 8.5), 7.07 (1H, d, J 8.5), 6.48 (1H, dd, J 8.5 and 2.2), 6.36 (1H, d, J 2.2), 5.22-5.17 (1H, m), 4.32-4.17 (2H, m), 3.69 (3H, s), 3.54-3.48 (4H, m), 2.84-2.77 (4H, m), 2.13-1.95 (2H, m).

### Example 94: (S)-N-(6-methoxychroman-4-yl)-2-(piperazin-1-yl)benzo[d]thiazole-6-carboxamide

Prepared using (S)-6-methoxychroman-4-amine in Step 3 of Scheme 6.

¹H NMR (hydrochloride salt form): δ_{H} (400 MHz, DMSO-d₆) 9.37 (2H, br. s), 8.79 (1H, br. d, J 8.1), 8.40 (1H, app. d, J1.7), 7.90 (1H, tented dd, J 8.5 and 1.7), 7.52 (1H, tented d, J 8.5), 6.79-6.72 (3H, m), 5.27-5.22 (1H, m), 4.28-4.21 (1H, m), 4.21-4.13 (1H, m), 3.87-3.22 (4H, m), 3.63 (3H, s), 3.30-2.22 (4H, m), 2.14-1.97 (2H, m).

### Example 95: (S)-N-(7-cyanochroman-4-yl)-2-(piperazin-1-yl)benzo[d]thiazole-6-carboxamide

Prepared using (S)-7-cyanochroman-4-amine in Step 3 of Scheme 6.

¹H NMR (free base form): δ_{H} (400 MHz, DMSO-d₆) 8.81 (1H, br. d, J 8.2), 8.31 (1H, app. d, J 1.8), 7.84 (1H, tented dd, J 8.5 and 1.8), 7.45 (1H, tented d, J 8.5), 7.38-7.35 (1H, m), 7.32-7.29 (2H, m), 5.37-5.31 (1H, m), 4.40-4.29 (2H, m), 3.54-3.49 (4H, m), 2.83-2.78 (4H, m), 2.55 (1H, br. s), 2.19-2.04 (2H, m).

### Examples 96 to 119

Examples 96 to 119 may be prepared according to the route shown in Scheme 7.

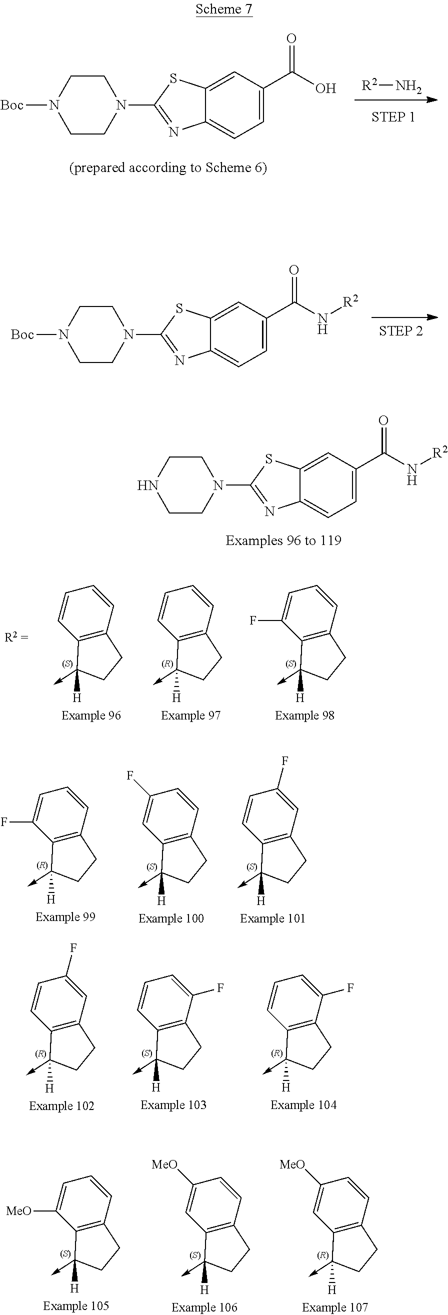

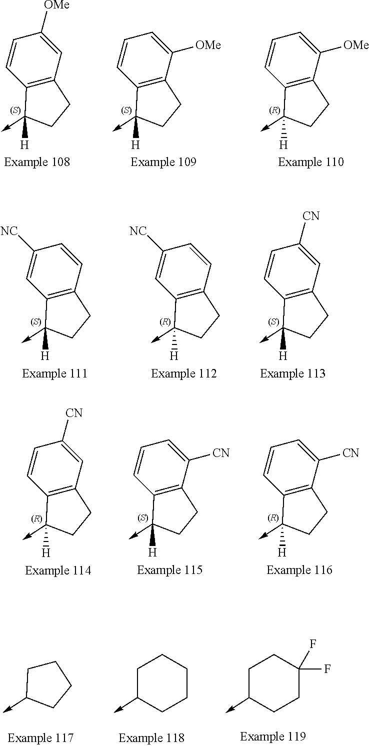

### Step 1 (Scheme 7): Synthesis of 2-(4-(tert-butoxycarbonyl)piperazin-1-yl)benzo[d]thiazole-6-carboxamide derivative

To a solution of 2-(4-(tert-butoxycarbonyl)piperazin-1-yl)benzo[d]thiazole-6-carboxylic acid (1.0 equiv.; prepared according to Scheme 6) in DCM (0.05 M in substrate) at 0° C. was added n-propylphosphonic acid anhydride cyclic trimer (50% in EtOAc; 2.0 equiv.) and DIPEA (4.0 equiv.). The mixture was stirred at 0° C. for 15 min prior to addition of the amine component [(1.2 equiv.) sourced commercially or prepared according to General Procedure 1 in the case of the (R)- or (S)-indan-1-amine derivatives]. The reaction mixture was brought to ambient temperature, stirring for 16 h, and then diluted with DCM and washed with water followed by brine. The organic phase was dried (Na₂SO₄) and evaporated to afford a residue that was processed by preparative HPLC to afford the 2-(4-(tert-butoxycarbonyl)piperazin-1-yl)benzo[d]thiazole-6-carboxamide derivative.

### Step 2 (Scheme 7) leading to examples 96 to 119: Boc deprotection

To a stirred solution of the 2-(4-(tert-butoxycarbonyl)piperazin-1-yl)benzo[d]thiazole-6-carboxamide derivative from Step 1 (1.0 equiv.) in DCM (0.1 M in substrate) at 0° C. was added 2 N HCl in diethyl ether (5.0 equiv. HCl). The reaction mixture was stirred at ambient temperature for 4 h (monitored by TLC). After consumption of starting material, the mixture was evaporated and the resulting residue triturated with hexanes and then lyophilized to afford the hydrochloride salt of the 2-(piperazin-1-yl)benzo[d]thiazole-6-carboxamide derivative as an off-white solid. The hydrochloride salt may optionally be desalted by partition between aqueous base and organic solvent, with the organic phase dried (Na₂SO₄) and evaporated to yield the free base form.

### Example 96: (S)-N-(2,3-dihydro-1H-inden-1-yl)-2-(piperazin-1-yl)benzo[d]thiazole-6-carboxamide

Prepared using (S)-1-aminoindane in Step 1 of Scheme 7.

¹H NMR (free base form): δ_{H} (400 MHz, DMSO-d₆) 8.66 (1H, br. d, J8.2), 8.32 (1H, app. s), 7.85 (1H, tented app. d, J 8.5), 7.44 (1H, tented d, J 8.5), 7.30-7.15 (4H, m), 5.59-5.53 (1H, m), 3.55-3.48 (4H, m), 3.04-2.95 (1H, m), 2.90-2.76 (5H, m), 2.50-2.40 (1H, m), 2.04-1.93 (1H, m).

### Example 97: (R)-N-(2,3-dihydro-1H-inden-1-yl)-2-(piperazin-1-yl)benzo[d]thiazole-6-carboxamide

Prepared using (R)-1-aminoindane in Step 1 of Scheme 7.

¹H NMR (hydrochloride salt form): δ_{H} (400 MHz, DMSO-d₆) 9.27 (2H, br. s), 8.70 (1H, br. d, J 7.8), 8.39 (1H, app. s), 7.90 (1H, tented app. d, J 8.3), 7.52 (1H, tented d, J 8.3), 7.34-7.12 (4H, m), 5.60-5.52 (1H, m), 3.89-3.80 (4H, m), 3.32-3.20 (4H, m), 3.06-2.94 (1H, m), 2.92-2.78 (1H, m), 2.50-2.40 (1H, m), 2.07-1.90 (1H, m).

### Example 98: (S)-N-(7-fluoro-2,3-dihydro-1H-inden-1-yl)-2-(piperazin-1-yl)benzo[d]thiazole-6-carboxamide

Prepared using (S)-7-fluoro-2,3-dihydro-1H-inden-1-amine in Step 1 of Scheme 7.

¹H NMR (hydrochloride salt form): δ_{H} (400 MHz, DMSO-d₆) 9.36 (2H, br. s), 8.80 (1H, br. d, J 8.3), 8.34 (1H, app. s), 7.85 (1H, tented app. d, J 8.5), 7.51 (1H, tented d, J 8.5), 7.32-7.27 (1H, m), 7.12 (1H, d, J7.4), 6.96 (1H, t, J 8.3), 5.78-5.72 (1H, m), 3.90-3.80 (4H, m), 3.30-3.22 (4H, m), 3.14-3.06 (1H, m), 2.91-2.83 (1H, m), 2.50-2.40 (1H, m), 2.04-1.94 (1H, m).

### Example 99: (R)-N-(7-fluoro-2,3-dihydro-1H-inden-1-yl)-2-(piperazin-1-yl)benzo[d]thiazole-6-carboxamide

Prepared using (R)-7-fluoro-2,3-dihydro-1H-inden-1-amine in Step 1 of Scheme 7.

¹H NMR (hydrochloride salt form): δ_{H} (400 MHz, DMSO-d₆) 9.17 (2H, br. s), 8.79 (1H, br. d, J 8.1), 8.34 (1H, app. s), 7.85 (1H, tented app. d, J 8.4), 7.51 (1H, tented d, J 8.4), 7.32-7.27 (1H, m), 7.13 (1H, d, J7.4), 6.96 (1H, t, J8.4), 5.78-5.72 (1H, m), 3.87-3.78 (4H, m), 3.31-3.22 (4H, m), 3.14-3.06 (1H, m), 2.91-2.83 (1H, m), 2.50-2.40 (1H, m), 2.04-1.94 (1H, m).

### Example 100: (S)-N-(6-fluoro-2,3-dihydro-1H-inden-1-yl)-2-(piperazin-1-yl)benzo[d]thiazole-6-carboxamide

Prepared using (S)-6-fluoro-2,3-dihydro-1H-inden-1-amine in Step 1 of Scheme 7.

¹H NMR (hydrochloride salt form): δ_{H} (400 MHz, DMSO-d₆) 9.41 (2H, br. s), 8.77 (1H, br. d, J 8.0), 8.40 (1H, app. d, J 1.7), 7.89 (1H, tented dd, J 8.5 and 1.7), 7.53 (1H, tented d, J 8.5), 7.28 (1H, dd, J 8.0 and 5.3), 7.06-6.99 (2H, m), 5.56-5.50 (1H, m), 3.89-3.81 (4H, m), 3.30-3.22 (4H, m), 3.00-2.93 (1H, m), 2.86-2.77 (1H, m), 2.50-2.40 (1H, m), 2.09-1.99 (1H, m).

### Example 101: (S)-N-(5-fluoro-2,3-dihydro-1H-inden-1-yl)-2-(piperazin-1-yl)benzo[d]thiazole-6-carboxamide

Prepared using (S)-5-fluoro-2,3-dihydro-1H-inden-1-amine in Step 1 of Scheme 7.

¹H NMR (hydrochloride salt form): δ_{H} (400 MHz, DMSO-d₆) 9.46 (2H, br. s), 8.72 (1H, br. d, J 8.2), 8.39 (1H, app. d, J 1.7), 7.89 (1H, tented dd, J 8.5 and 1.7), 7.52 (1H, tented d, J 8.5), 7.25 (1H, dd, J 8.1 and 5.5), 7.10 (1H, dd, J 9.2 and 2.3), 7.01-6.96 (1H, m), 5.54-5.48 (1H, m), 3.89-3.82 (4H, m), 3.30-3.21 (4H, m), 3.01 (1H, tented ddd, J 16.2, 8.7 and 3.3), 2.85 (1H, tented app. dt, J 16.2 and 8.1), 2.50-2.40 (1H, m), 2.04 (1H, app. dq, J 12.6 and 8.1).

### Example 102: (R)-N-(5-fluoro-2,3-dihydro-1H-inden-1-yl)-2-(piperazin-1-yl)benzo[d]thiazole-6-carboxamide

Prepared using (R)-5-fluoro-2,3-dihydro-1H-inden-1-amine in Step 1 of Scheme 7.

¹H NMR (hydrochloride salt form): δ_{H} (400 MHz, DMSO-d₆) 9.39 (2H, br. s), 8.71 (1H, br. d, J 8.2), 8.39 (1H, app. d, J 1.7), 7.89 (1H, tented dd, J 8.5 and 1.7), 7.52 (1H, tented d, J 8.5), 7.25 (1H, dd, J 8.1 and 5.5), 7.10 (1H, dd, J 9.2 and 2.3), 7.01-6.96 (1H, m), 5.54-5.48 (1H, m), 3.89-3.82 (4H, m), 3.30-3.21 (4H, m), 3.01 (1H, tented ddd, J 16.2, 8.7 and 3.3), 2.85 (1H, tented app. dt, J 16.2 and 8.1), 2.50-2.40 (1H, m), 2.04 (1H, app. dq, J 12.6 and 8.1).

### Example 103: (S)-N-(4-fluoro-2,3-dihydro-1H-inden-1-yl)-2-(piperazin-1-yl)benzo[d]thiazole-6-carboxamide

Prepared using (S)-4-fluoro-2,3-dihydro-1H-inden-1-amine in Step 1 of Scheme 7.

¹H NMR (hydrochloride salt form): δ_{H} (400 MHz, DMSO-d₆) 9.29 (2H, br. s), 8.78 (1H, br. d, J 8.2), 8.39 (1H, app. d, J 1.7), 7.89 (1H, tented dd, J 8.5 and 1.7), 7.53 (1H, tented d, J 8.5), 7.27-7.21 (1H, m), 7.10-7.03 (2H, m), 5.63-5.57 (1H, m), 3.88-3.82 (4H, m), 3.30-3.22 (4H, m), 3.06 (1H, tented ddd, J 16.2, 8.7 and 3.3), 2.85 (1H, tented app. dt, J 16.2 and 8.1), 2.50-2.40 (1H, m), 2.05 (1H, app. dq, J12.6 and 8.1).

### Example 104: (R)-N-(4-fluoro-2,3-dihydro-1H-inden-1-yl)-2-(piperazin-1-yl)benzo[d]thiazole-6-carboxamide

Prepared using (R)-4-fluoro-2,3-dihydro-1H-inden-1-amine in Step 1 of Scheme 7.

¹H NMR (hydrochloride salt form): δ_{H} (400 MHz, DMSO-d₆) 9.28 (2H, br. s), 8.50 (1H, br. d, J 7.9), 8.33 (1H, app. d, J 1.7), 7.89 (1H, tented dd, J 8.5 and 1.7), 7.53 (1H, tented d, J 8.5), 7.27-7.21 (1H, m), 7.10-7.03 (2H, m), 5.64-5.58 (1H, m), 3.88-3.82 (4H, m), 3.31-3.24 (4H, m), 3.07 (1H, tented ddd, J 16.2, 8.7 and 3.3), 2.86 (1H, tented app. dt, J 16.2 and 8.1), 2.50-2.40 (1H, m), 2.06 (1H, app. dq, J 12.6 and 8.1).

### Example 105: (S)-N-(7-methoxy-2,3-dihydro-1H-inden-1-yl)-2-(piperazin-1-yl)benzo[d]thiazole-6-carboxamide

Prepared using (S)-7-methoxy-2,3-dihydro-1H-inden-1-amine in Step 1 of Scheme 7.

¹H NMR (hydrochloride salt form): δ_{H} (400 MHz, DMSO-d₆) 9.37 (2H, br. s), 8.51 (1H, d, J8.0), 8.33 (1H, app. s), 7.84 (1H, tented app. d, J 8.4), 7.49 (1H, tented d, J 8.4), 7.24 (1H, app. t, J 7.9), 6.88 (1H, d, J 7.6), 6.80 (1H, d, J 8.2), 5.57-5.52 (1H, m), 3.88-3.81 (4H, m), 3.70 (3H, s), 3.30-3.22 (4H, m), 3.07 (1H, tented dt, J 15.8 and 7.9), 2.78 (1H, tented ddd, J 15.8, 8.6 and 4.5), 2.42-2.32 (1H, m), 1.95-1.86 (1H, m).

### Example 106: (S)-N-(6-methoxy-2,3-dihydro-1H-inden-1-yl)-2-(piperazin-1-yl)benzo[d]thiazole-6-carboxamide

Prepared using (S)-6-methoxy-2,3-dihydro-1H-inden-1-amine in Step 1 of Scheme 7.

¹H NMR (hydrochloride salt form): δ_{H} (400 MHz, DMSO-d₆) 9.57 (2H, br. s), 8.71 (1H, br. d, J 8.2), 8.40 (1H, app. d, J 1.6), 7.90 (1H, tented dd, J 8.5 and 1.6), 7.53 (1H, tented d, J 8.5), 7.16 (1H, d, J 7.9), 6.82-6.76 (2H, m), 5.55-5.49 (1H, m), 3.90-3.83 (4H, m), 3.69 (3H, s), 3.30-3.22 (4H, m), 2.92 (1H, tented ddd, J 15.4, 8.7 and 2.9), 2.76 (1H, tented app. dt, J 15.4 and 7.7), 2.48-2.41 (1H, m), 1.99 (1H, app. dq, J 12.5 and 8.7).

### Example 107: (R)-N-(6-methoxy-2,3-dihydro-1H-inden-1-yl)-2-(piperazin-1-yl)benzo[d]thiazole-6-carboxamide

Prepared using (R)-6-methoxy-2,3-dihydro-1H-inden-1-amine in Step 1 of Scheme 7.

¹H NMR (hydrochloride salt form): δ_{H} (400 MHz, DMSO-d₆) 9.51 (2H, br. s), 8.70 (1H, br. d, J 7.9), 8.40 (1H, app. d, J 1.6), 7.90 (1H, tented dd, J 8.5 and 1.6), 7.52 (1H, tented d, J 8.5), 7.16 (1H, d, J 7.9), 6.82-6.76 (2H, m), 5.55-5.49 (1H, m), 3.90-3.83 (4H, m), 3.69 (3H, s), 3.30-3.22 (4H, m), 2.92 (1H, tented ddd, J 15.4, 8.7 and 2.9), 2.76 (1H, tented app. dt, J 15.4 and 7.7), 2.48-2.41 (1H, m), 1.99 (1H, app. dq, J 12.5 and 8.7).

### Example 108: (S)-N-(5-methoxy-2,3-dihydro-1H-inden-1-yl)-2-(piperazin-1-yl)benzo[d]thiazole-6-carboxamide

Prepared using (S)-5-methoxy-2,3-dihydro-1H-inden-1-amine in Step 1 of Scheme 7.

¹H NMR (free base form): δ_{H} (400 MHz, DMSO-d₆) 8.58 (1H, br. d, J8.1), 8.33 (1H, app. d, J 1.7), 7.85 (1H, tented dd, J 8.5 and 1.7), 7.46 (1H, tented d, J 8.5), 7.14 (1H, d, J 8.3), 6.84 (1H, d, J2.2), 6.74 (1H, dd, J 8.3 and 2.2), 5.51-5.45 (1H, m), 3.72 (3H, s), 3.64-3.59 (4H, m), 2.98 (1H, tented ddd, J 16.2, 8.7 and 3.5), 2.97-2.91 (4H, m), 2.81 (1H, tented app. dt, J 16.2 and 8.1), 2.50-2.40 (1H, m), 2.03-1.99 (1H, m).

### Example 109: (S)-N-(4-methoxy-2,3-dihydro-1H-inden-1-yl)-2-(piperazin-1-yl)benzo[d]thiazole-6-carboxamide

Prepared using (S)-4-methoxy-2,3-dihydro-1H-inden-1-amine in Step 1 of Scheme 7.

¹H NMR (hydrochloride salt form): δ_{H} (400 MHz, DMSO-d₆) 9.28 (2H, br. s), 8.69 (1H, br. d, J 8.3), 8.38 (1H, app. d, J 1.6), 7.88 (1H, tented dd, J 8.5 and 1.6), 7.52 (1H, tented d, J 8.5), 7.17 (1H, t, J7.8), 6.84 (2H, d, J7.8), 5.59-5.53 (1H, m), 3.87-3.82 (4H, m), 3.55 (3H, s), 3.30-3.23 (4H, m), 2.94 (1H, tented ddd, J 16.0, 9.0 and 3.0), 2.81 (1H, tented app. dt, J 16.0 and 8.0), 2.50-2.40 (1H, m), 1.97 (1H, app. dq, J 12.5 and 8.7).

### Example 110: (R)-N-(4-methoxy-2,3-dihydro-1H-inden-1-yl)-2-(piperazin-1-yl)benzo[d]thiazole-6-carboxamide

Prepared using (R)-4-methoxy-2,3-dihydro-1H-inden-1-amine in Step 1 of Scheme 7.

¹H NMR (hydrochloride salt form): δ_{H} (400 MHz, DMSO-d₆) 9.31 (2H, br. s), 8.69 (1H, br. d, J 8.4), 8.38 (1H, app. d, J 1.6), 7.88 (1H, tented dd, J 8.5 and 1.6), 7.52 (1H, tented d, J 8.5), 7.17 (1H, t, J7.8), 6.84 (2H, d, J7.8), 5.59-5.53 (1H, m), 3.87-3.82 (4H, m), 3.55 (3H, s), 3.30-3.23 (4H, m), 2.94 (1H, tented ddd, J 16.0, 9.0 and 3.0), 2.81 (1H, tented app. dt, J 16.0 and 8.0), 2.50-2.40 (1H, m), 1.97 (1H, app. dq, J 12.5 and 8.7).

### Example 111: (S)-N-(6-cyano-2,3-dihydro-1H-inden-1-yl)-2-(piperazin-1-yl)benzo[d]thiazole-6-carboxamide

Prepared using (S)-6-cyano-2,3-dihydro-1H-inden-1-amine in Step 1 of Scheme 7.

¹H NMR (free base form): δ_{H} (400 MHz, DMSO-d₆) 8.76 (1H, br. d, J8.2), 8.31 (1H, app. d, J 1.7), 7.84 (1H, tented dd, J 8.5 and 1.7), 7.75 (1H, app. s), 7.64 (1H, app. d, J7.8), 7.45 (1H, tented d, J 8.5), 6.41 (1H, d, J 7.8), 5.62-5.56 (1H, m), 3.53-3.49 (4H, m), 3.04 (1H, tented ddd, J 16.2, 8.8 and 2.7), 2.90 (1H, tented app. dt, J 16.2 and 8.1), 2.82-2.78 (4H, m), 2.50-2.44 (1H, m), 2.05 (1H, app. dq, J 12.6 and 8.9).

### Example 112: (R)-N-(6-cyano-2,3-dihydro-1H-inden-1-yl)-2-(piperazin-1-yl)benzo[d]thiazole-6-carboxamide

Prepared using (R)-6-cyano-2,3-dihydro-1H-inden-1-amine in Step 1 of Scheme 7.

¹H NMR (free base form): δ_{H} (400 MHz, DMSO-d₆) 8.75 (1H, br. d, J 8.2), 8.31 (1H, app. d, J 1.7), 7.84 (1H, tented dd, J 8.5 and 1.7), 7.75 (1H, app. s), 7.64 (1H, app. d, J 8.0), 7.45 (1H, tented d, J 8.5), 6.41 (1H, d, J 8.0), 5.62-5.56 (1H, m), 3.53-3.49 (4H, m), 3.04 (1H, tented ddd, J 16.2, 8.8 and 2.7), 2.90 (1H, tented app. dt, J 16.2 and 8.1), 2.82-2.78 (4H, m), 2.50-2.44 (1H, m), 2.05 (1H, app. dq, J 12.5 and 8.8).

### Example 113: (S)-N-(5-cyano-2,3-dihydro-1H-inden-1-yl)-2-(piperazin-1-yl)benzo[d]thiazole-6-carboxamide

Prepared using (S)-5-cyano-2,3-dihydro-1H-inden-1-amine in Step 1 of Scheme 7.

¹H NMR (free base form): δ_{H} (400 MHz, DMSO-d₆) 8.73 (1H, br. d, J8.0), 8.32 (1H, app. d, J 1.7), 7.85 (1H, tented dd, J 8.5 and 1.7), 7.69 (1H, app. d, J7.8), 7.67 (1H, app. s), 7.49 (1H, app. d, J 7.8), 7.45 (1H, tented d, J 8.5), 5.59-5.53 (1H, m), 3.54-3.49 (4H, m), 3.08 (1H, tented ddd, J 16.8, 8.8 and 3.2), 2.90 (1H, tented app. dt, J 16.8 and 8.4), 2.83-2.78 (4H, m), 2.54-2.48 (1H, m), 2.03 (1H, app. dq, J 12.6 and 8.4).

### Example 114: (R)-N-(5-cyano-2,3-dihydro-1H-inden-1-yl)-2-(piperazin-1-yl)benzo[d]thiazole-6-carboxamide

Prepared using (R)-5-cyano-2,3-dihydro-1H-inden-1-amine in Step 1 of Scheme 7.

¹H NMR (free base form): δ_{H} (400 MHz, DMSO-d₆) 8.73 (1H, br. d, J7.9), 8.32 (1H, app. d, J 1.7), 7.85 (1H, tented dd, J 8.5 and 1.7), 7.69 (1H, app. d, J7.8), 7.67 (1H, app. s), 7.49 (1H, app. d, J 7.8), 7.45 (1H, tented d, J 8.5), 5.59-5.53 (1H, m), 3.54-3.49 (4H, m), 3.08 (1H, tented ddd, J 16.8, 8.8 and 3.2), 2.90 (1H, tented app. dt, J 16.8 and 8.4), 2.83-2.78 (4H, m), 2.54-2.48 (1H, m), 2.03 (1H, app. dq, J 12.6 and 8.4).

### Example 115: (S)-N-(4-cyano-2,3-dihydro-1H-inden-1-yl)-2-(piperazin-1-yl)benzo[d]thiazole-6-carboxamide

Prepared using (S)-4-cyano-2,3-dihydro-1H-inden-1-amine in Step 1 of Scheme 7.

¹H NMR (hydrochloride salt form): δ_{H} (400 MHz, DMSO-d₆) 9.24 (2H, br. s), 8.81 (1H, br. d, J 8.2), 8.38 (1H, app. d, J 1.7), 7.88 (1H, tented dd, J8.5 and 1.7), 7.71 (1H, app. d, J 7.6), 7.57 (1H, tented app. d, J7.6), 7.53 (1H, tented d, J8.5), 7.40 (1H, tented t, J7.8), 5.65-5.59 (1H, m), 3.87-3.81 (4H, m), 3.30-3.23 (4H, m), 3.17 (1H, tented ddd, J16.4, 8.9 and 3.5), 3.03 (1H, tented app. dt, J 16.4 and 8.2), 2.50-2.40 (1H, m), 2.10 (1H, app. dq, J 12.6 and 8.4).

### Example 116: (R)-N-(4-cyano-2,3-dihydro-1H-inden-1-yl)-2-(piperazin-1-yl)benzo[d]thiazole-6-carboxamide

Prepared using (R)-4-cyano-2,3-dihydro-1H-inden-1-amine in Step 1 of Scheme 7.

¹H NMR (hydrochloride salt form): δ_{H} (400 MHz, DMSO-d₆) 9.35 (2H, br. s), 8.82 (1H, br. d, J 8.1), 8.38 (1H, app. d, J 1.7), 7.88 (1H, tented dd, J8.5 and 1.7), 7.70 (1H, app. d, J7.6), 7.58 (1H, tented app. d, J7.6), 7.53 (1H, tented d, J8.5), 7.40 (1H, tented t, J7.8), 5.65-5.59 (1H, m), 3.87-3.81 (4H, m), 3.30-3.23 (4H, m), 3.17 (1H, tented ddd, J16.4, 8.9 and 3.5), 3.03 (1H, tented app. dt, J 16.4 and 8.2), 2.50-2.40 (1H, m), 2.09 (1H, app. dq, J 12.6 and 8.4).

### Example 117: N-cyclopentyl-2-(piperazin-1-yl)benzo[d]thiazole-6-carboxamide

Prepared using cyclopentylamine in Step 1 of Scheme 7.

¹H NMR (free base form): δ_{H} (400 MHz, DMSO-d₆) 8.26 (1H, app. d, J 1.7), 8.19 (1H, br. d, J 7.2), 7.89 (1H, tented dd, J 8.5 and 1.7), 7.45 (1H, tented d, J 8.5), 4.26-4.17 (1H, m), 3.62-3.58 (4H, m), 2.96-2.91 (4H, m), 1.93-1.82 (2H, m), 1.76-1.63 (2H, m), 1.59-1.47 (4H, m).

### Example 118: N-cyclohexyl-2-(piperazin-1-yl)benzo[d]thiazole-6-carboxamide

Prepared using cyclohexylamine in Step 1 of Scheme 7.

¹H NMR (free base form): δ_{H} (400 MHz, DMSO-d₆) 8.24 (1H, app. d, J 1.7), 8.09 (1H, br. d, J 7.9), 7.77 (1H, tented dd, J 8.4 and 1.7), 7.43 (1H, tented d, J 8.4), 3.81-3.70 (1H, m), 3.55-3.46 (4H, m), 2.85-2.76 (4H, m), 1.87-1.77 (2H, m), 1.77-1.68 (2H, m), 1.65-1.56 (1H, m), 1.37-1.22 (4H, m), 1.21-1.05 (1H, m).

### Example 119: N-(4,4-difluorocyclohexyl)-2-(piperazin-1-yl)benzo[d]thiazole-6-carboxamide

Prepared using 4,4-difluorocyclohexylamine in Step 1 of Scheme 7.

¹H NMR (free base form): δ_{H} (400 MHz, DMSO-d₆) 8.25 (1H, app. d, J 1.7), 8.20 (1H, br. d, J 7.6), 7.78 (1H, tented dd, J 8.4 and 1.7), 7.44 (1H, tented d, J 8.4), 4.07-3.91 (1H, m), 3.55-3.46 (4H, m), 2.85-2.76 (4H, m), 2.16-1.75 (6H, m), 1.73-1.54 (2H, m).

### Example 120: (S)-N-(chroman-4-yl)-2-(4-(2-hydroxyethyl)piperazin-1-yl)benzo[d]thiazole-6-carboxamide

Example 120 may be prepared according to Scheme 8.

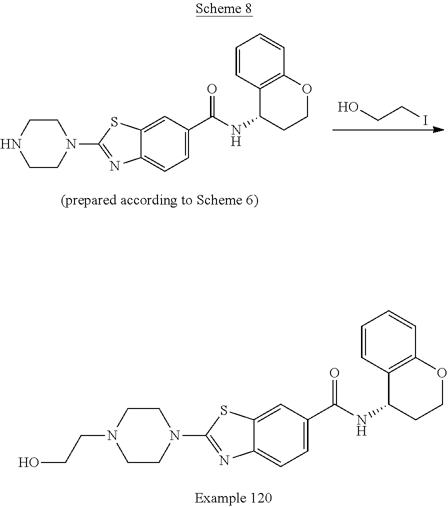

To a stirred mixture of (S)-N-(chroman-4-yl)-2-(piperazin-1-yl)benzo[d]thiazole-6-carboxamide hydrochloride (Example 83; 1.0 equiv.) in DMF (1 volume; 0.25 M in substrate) under Ar was added K₂CO₃ (2.0 equiv.) and 2-iodoethanol (2.0 equiv.). The reaction mixture was heated to 70° C., stirring for 16 h, and then cooled, diluted with water (5 volumes) and extracted with 5% v/v MeOH/DCM (3×10 volumes). The combined organic extract was washed with brine, dried (Na₂SO₄), and concentrated in vacuo to afford the crude product. The latter was processed by flash chromatography (5-10% v/v MeOH/DCM) and fractions containing the target material were combined and evaporated to afford (S)-N-(chroman-4-yl)-2-(4-(2-hydroxyethyl)piperazin-1-yl)benzo[d]thiazole-6-carboxamide as an off-white solid (44% yield).

¹H NMR: δ_{H} (400 MHz, DMSO-d₆) 8.76 (1H, br. d, J8.2), 8.34 (1H, app. s), 7.86 (1H, tented app. d, J8.5), 7.45 (1H, tented d, J 8.5), 7.21-7.12 (2H, m), 6.87 (1H, t, J7.4), 6.80 (1H, dd, J 8.1), 5.31-5.26 (1H, m), 4.48 (1H, br. s), 4.35-4.19 (2H, m), 3.63-3.50 (6H, m), 2.63-2.50 (4H, m), 2.50-2.37 (2H, m), 2.16-2.00 (2H, m).

### Example 121: 2-(3,8-diazabicyclo[3.2.1]octan-3-yl)-N-((S)-chroman-4-yl)benzo[d]thiazole-6-carboxamide

Prepared according to the method of Example 83, using tert-butyl 3,8-diazabicyclo[3.2.1]octane-8-carboxylate instead of tert-butyl piperazine-1-carboxylate in Step 1 of Scheme 6.

¹H NMR (free base form): δ_{H} (400 MHz, DMSO-d₆) 8.75 (1H, br. d, J 8.2), 8.33 (1H, app. d, J 1.8), 7.86 (1H, tented dd, J 8.6 and 1.8), 7.43 (1H, tented d, J 8.5), 7.20-7.12 (2H, m), 6.87 (1H, td, J 7.5 and 1.1), 6.79 (1H, dd, J8.2 and 1.1), 5.31-5.26 (1H, m), 4.35-4.19 (2H, m), 3.71-3.57 (2H, m), 3.54-3.48 (2H, m), 3.26 (2H, dd, J 11. 6 and 2.0), 2.55 (1H, br. s), 2.16-2.00 (2H, m), 1.75-1.57 (4H, m).

### Example 122: 2-(3,8-diazabicyclo[3.2.1]octan-8-yl)-N-((S)-chroman-4-yl)benzo[d]thiazole-6-carboxamide

Prepared according to the method of Example 83, using tert-butyl 3,8-diazabicyclo[3.2.1]octane-3-carboxylate instead of tert-butyl piperazine-1-carboxylate in Step 1 of Scheme 6.

¹H NMR (free base form): δ_{H} (400 MHz, DMSO-d₆) 8.75 (1H, br. d, J 8.2), 8.32 (1H, app. d, J 1.8), 7.85 (1H, tented dd, J 8.6 and 1.8), 7.44 (1H, tented d, J 8.5), 7.20-7.12 (2H, m), 6.87 (1H, td, J 7.5 and 1.1), 6.80 (1H, dd, J8.2 and 1.1), 5.31-5.26 (1H, m), 4.35-4.17 (4H, m), 2.95 (2H, tented app. d, J 12.5), 2.62 (2H, tented app. d, J 12.5), 2.16-1.90 (6H, m). [Diazabicyclo[3.2.1]octane NH not identifiable as a distinct resonance in this solvent.]

### Examples 123 to 141

Examples 123 to 141 may be prepared according to the route shown in Scheme 9.

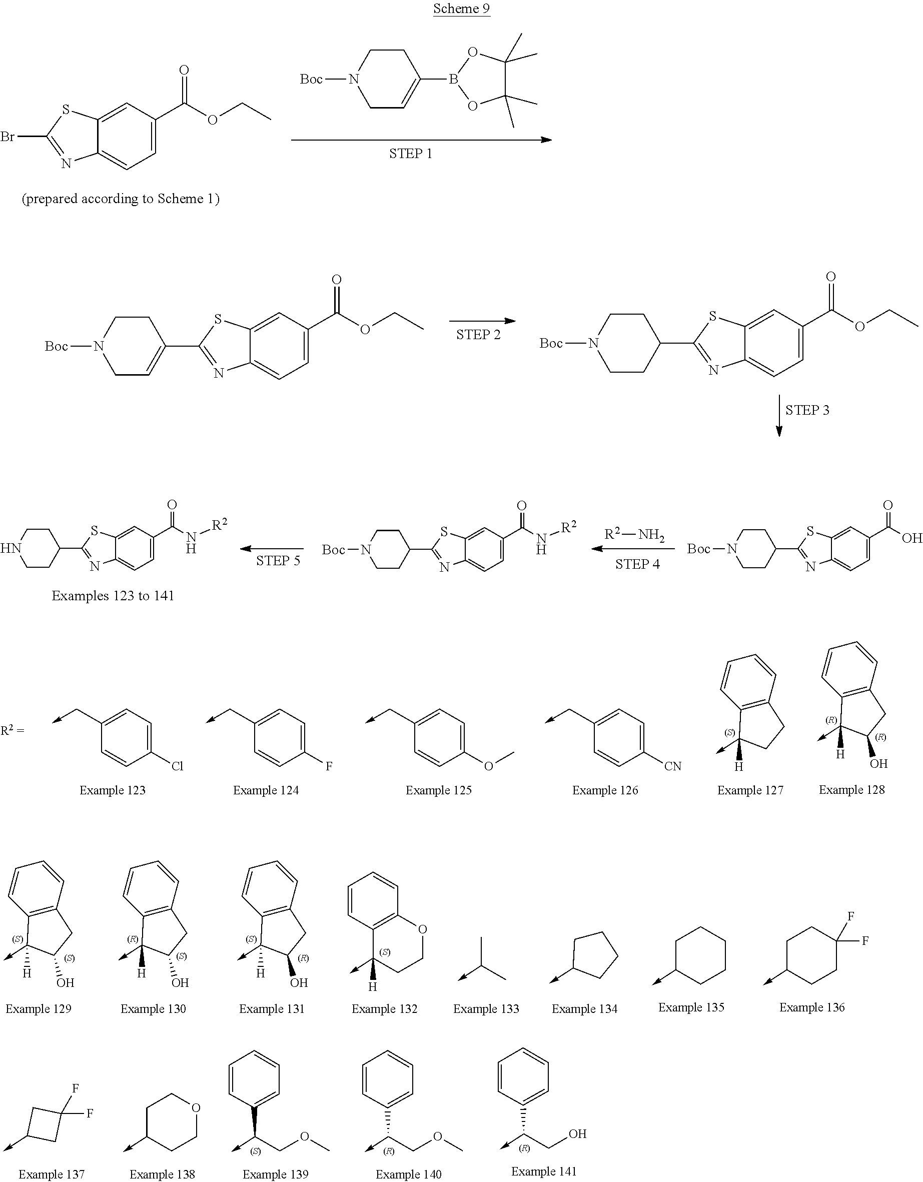

### Step 1 (Scheme 9): Synthesis of ethyl 2-(1-(tert-butoxycarbonyl)-1,2,3,6-tetrahydropyridin-4-yl)benzo[d]thiazole-6-carboxylate

To an argon-purged mixture of ethyl 2-bromobenzo[d]thiazole-6-carboxylate (1.0 equiv.; prepared according to Scheme 1) in 10:1 v/v 1,4-dioxane/water (0.13 M in substrate) was added tert-butyl 4-(4,4,5,5-tetramethyl-1,3,2-dioxaborolan-2-yl)-3,6-dihydropyridine-1 (2H)-carboxylate (1.2 equiv.), K₂CO₃ (2.0 equiv.) and Pd(PPh₃)₄ (10 mol %). The mixture was heated to reflux under Ar for 16 h and then cooled and filtered through Celite®, washing with 5% v/v MeOH/DCM. The filtrate was concentrated under reduced pressure to obtain a solid residue that was dissolved in 5% v/v MeOH/DCM and washed with water followed by brine. The organic layer was dried (Na₂SO₄) and concentrated under reduced pressure. The crude product was processed by flash column chromatography (70% v/v EtOAc/hexanes) to obtain ethyl 2-(1-(tert-butoxycarbonyl)-1,2,3,6-tetrahydropyridin-4-yl)benzo[d]thiazole-6-carboxylate as a light brown solid (63% yield).

### Step 2 (Scheme 9): Synthesis of ethyl 2-(1-(tert-butoxycarbonyl)-1,2,3,6-tetrahydropyridin-4-yl)benzo[d]thiazole-6-carboxylate

A solution of ethyl 2-(1-(tert-butoxycarbonyl)-1,2,3,6-tetrahydropyridin-4-yl)benzo[d]thiazole-6-carboxylate in ethanol (0.07 M in substrate) was hydrogenated at ambient temperature and pressure over PtO₂. After 16 h the reaction mixture was purged with Ar and filtered through Celite®, washing with MeOH, and the filtrate concentrated under reduced pressure to obtain a solid residue. The latter was triturated with hexanes and dried to obtain ethyl 2-(1-(tert-butoxycarbonyl)piperidin-4-yl)benzo[d]thiazole-6-carboxylate as a brown solid (98% yield).

### Step 3 (Scheme 9): Synthesis of 2-(1-(tert-butoxycarbonyl)piperidin-4-yl)benzo[d]thiazole-6-carboxylic acid

To a stirred mixture of ethyl 2-(1-(tert-butoxycarbonyl)piperidin-4-yl)benzo[d]thiazole-6-carboxylate (1.0 equiv.) in 3:3:1 v/v/v THF/MeOH/water (0.3 M in substrate) was added LiOH·H₂O (2.0 equiv.). The reaction mixture was stirred at ambient temperature for 16 h and then concentrated under reduced pressure, diluted with ice-cold water and acidified with aq. citric acid solution. The precipitated solid was collected by filtration and dried to afford 2-(1-(tert-butoxycarbonyl)piperidin-4-yl)benzo[d]thiazole-6-carboxylic acid as a brown solid (69% yield).

### Step 4 (Scheme 9): General procedure for amide coupling

To a mixture of 2-(1-(tert-butoxycarbonyl)piperidin-4-yl)benzo[d]thiazole-6-carboxylic acid (1.0 equiv.) in DCM (0.1 M in substrate) at 0° C. was added n-propylphosphonic acid anhydride cyclic trimer (50% in EtOAc; 2.0 equiv.) and DIPEA (4.0 equiv.). The mixture was stirred at 0° C. for 15 min prior to addition of the amine component (1.2 equiv.). The reaction mixture was brought to ambient temperature, stirring for 16 h, and then diluted with DCM and washed with water followed by brine. The organic phase was dried (Na₂SO₄) and evaporated to afford a residue that was used in the next step without purification.

### Step 5 (Scheme 9) leading to Examples 123 to 141: General procedure for Boc-deprotection

To a stirred solution of the benzo[d]thiazole-6-carboxamide derivative from Step 4 (1.0 equiv.) in DCM (0.2 M in substrate) at 0° C. was added 4 M HCl in 1,4-dioxane (8.0 equiv. HCl). The reaction mixture was stirred at ambient temperature for 16 h (monitored by TLC). After consumption of starting material, the mixture was evaporated and the resulting residue triturated with hexanes and then lyophilized to afford the hydrochloride salt of the 2-(piperidin-4-yl)benzo[d]thiazole-6-carboxamide derivative or optionally desalted and processed by preparative HPLC to isolate it in free base form. Examples 123 to 141 were obtained as off-white solids.

### Example 123: N-(4-chlorobenzyl)-2-(piperidin-4-yl)benzo[d]thiazole-6-carboxamide

Prepared using 4-chlorobenzylamine as the amine component.

¹H NMR (free base form): δ_{H} (400 MHz, DMSO-d₆) 9.17 (1H, br. t, J5.9), 8.59 (1H, dd, J 1.6 and 0.7), 8.01 (1H, tented dd, J 8.6 and 0.7), 7.98 (1H, tented dd, J 8.6 and 1.6), 7.41-7.34 (4H, AA′BB′ m), 4.49 (2H, d, J 5.9), 3.22 (1H, tt, J 11.6 and 3.9), 3.06-2.99 (2H, m), 2.62 (2H, td, J 11.8 and 1.9), 2.06-1.99 (2H, m), 1.66 (2H, qd, J 12.2 and 3.6). [piperidine NH not identifiable as a distinct resonance in this solvent.]

### Example 124: N-(4-fluorobenzyl)-2-(piperidin-4-yl)benzo[d]thiazole-6-carboxamide

Prepared using 4-fluorobenzylamine as the amine component.

¹H NMR (free base form): δ_{H} (400 MHz, DMSO-d₆) 9.16 (1H, br. t, J 5.9), 8.59 (1H, app. s), 8.02-7.97 (2H, m), 7.41-7.35 (2H, m), 7.19-7.11 (2H, m), 4.48 (2H, d, J 5.9), 3.22 (1H, tt, J 11.6 and 3.9), 3.06-2.99 (2H, m), 2.62 (2H, td, J 11.8 and 1.9), 2.06-1.99 (2H, m), 1.66 (2H, qd, J 12.2 and 3.6). [piperidine NH not identifiable as a distinct resonance in this solvent.]

### Example 125: N-(4-methoxybenzyl)-2-(piperidin-4-yl)benzo[d]thiazole-6-carboxamide

Prepared using 4-methoxybenzylamine as the amine component.

¹H NMR (free base form): δ_{H} (400 MHz, DMSO-d₆) 9.08 (1H, br. t, J 5.8), 8.58 (1H, app. s), 8.01-7.97 (2H, m), 7.29-7.25 (2H, AA′BB′ m), 6.91-6.87 (2H, AA′BB′ m), 4.43 (2H, d, J 5.8), 3.72 (3H, s), 3.22 (1H, tt, J 11.6 and 3.9), 3.06-2.99 (2H, m), 2.62 (2H, td, J 12.0 and 1.9), 2.06-1.99 (2H, m), 1.66 (2H, qd, J 12.1 and 3.6). [piperidine NH not identifiable as a distinct resonance in this solvent.]

### Example 126: N-(4-cyanobenzyl)-2-(piperidin-4-yl)benzo[d]thiazole-6-carboxamide

Prepared using 4-cyanobenzylamine as the amine component.

¹H NMR (free base form): δ_{H} (400 MHz, DMSO-d₆) 9.28 (1H, br. t, J 5.9), 8.61 (1H, app. s), 8.03-7.98 (2H, m), 7.84-7.78 (2H, AA′BB′ m), 7.56-7.51 (2H, AA′BB′ m), 4.58 (2H, d, J 5.9), 3.23 (1H, tt, J 11.6 and 3.9), 3.06-2.99 (2H, m), 2.62 (2H, td, J 12.0 and 1.9), 2.06-1.99 (2H, m), 1.66 (2H, qd, J 12.1 and 3.6). [piperidine NH not identifiable as a distinct resonance in this solvent.]

### Example 127: (S)-N-(2,3-dihydro-1H-inden-1-yl)-2-(piperidin-4-yl)benzo[d]thiazole-6-carboxamide

Prepared using (S)-1-aminoindane as the amine component.

¹H NMR (free base form): δ_{H} (400 MHz, DMSO-d₆) 8.87 (1H, br. d, J8.1), 8.60 (1H, app. s), 8.03-7.97 (2H, m), 7.30-7.14 (4H, m), 5.60-5.54 (1H, m), 3.22 (1H, tt, J 11.6 and 3.9), 3.06-2.95 (3H, m), 2.85 (1H, tented app. dt, J 15.8 and 7.9), 2.62 (2H, td, J 12.0 and 1.9), 2.50-2.40 (1H, m), 2.07-1.94 (3H, m), 1.66 (2H, qd, J 12.1 and 3.6). [piperidine NH not identifiable as a distinct resonance in this solvent.]

### Example 128: N-((1R,2R)-2-hydroxy-2,3-dihydro-1H-inden-1-yl)-2-(piperidin-4-yl)benzo[d]thiazole-6-carboxamide

Prepared using (1R,2R)-1-amino-2-indanol as the amine component.

¹H NMR (free base form): δ_{H} (400 MHz, DMSO-d₆) 8.85 (1H, br. d, J 8.5), 8.64 (1H, app. d, J 1.6), 8.05 (1H, tented dd, J 8.6 and 1.6), 8.01 (1H, tented d, J 8.6), 7.26-7.11 (4H, m), 5.37 (1H, br. d, J 5.7), 5.34-5.31 (1H, m), 4.48-4.41 (1H, m), 3.26 (1H, tt, J 11.6 and 3.9), 3.22 (1H, dd, J 15.6 and 7.3), 3.10-3.04 (2H, m), 2.76 (1H, dd, J 15.6 and 7.5), 2.67 (2H, td, J 12.0 and 1.9), 2.10-2.02 (2H, m), 1.70 (2H, qd, J 12.1 and 3.6). [piperidine NH not identifiable as a distinct resonance in this solvent.]

### Example 129: N-((1S,2S)-2-hydroxy-2,3-dihydro-1H-inden-1-yl)-2-(piperidin-4-yl)benzo[d]thiazole-6-carboxamide

Prepared using (1S,2S)-1-amino-2-indanol as the amine component.

¹H NMR (free base form): δ_{H} (400 MHz, DMSO-d₆) 8.87 (1H, br. d, J8.4), 8.64 (1H, app. d, J 1.6), 8.05 (1H, tented dd, J 8.6 and 1.6), 8.01 (1H, tented d, J 8.6), 7.26-7.11 (4H, m), 5.40 (1H, br. d, J 5.1), 5.34-5.30 (1H, m), 4.48-4.41 (1H, m), 3.24 (1H, tt, J 11.6 and 3.9), 3.19 (1H, dd, J 15.6 and 7.3), 3.08-3.00 (2H, m), 2.76 (1H, dd, J 15.6 and 7.5), 2.64 (2H, td, J 12.0 and 1.9), 2.08-2.00 (2H, m), 1.68 (2H, qd, J 12.1 and 3.6). [piperidine NH not identifiable as a distinct resonance in this solvent.]

### Example 130: N-((1R,2S)-2-hydroxy-2,3-dihydro-1H-inden-1-yl)-2-(piperidin-4-yl)benzo[d]thiazole-6-carboxamide

Prepared using (1R,2S)-1-amino-2-indanol as the amine component.

¹H NMR (free base form): δ_{H} (400 MHz, DMSO-d₆) 8.70 (1H, app. d, J 1.6), 8.40 (1H, br. d, J 8.4), 8.07 (1H, tented dd, J 8.5 and 1.6), 7.99 (1H, tented d, J 8.5), 7.31-7.16 (4H, m), 5.47 (1H, dd, J 8.4 and 5.2), 5.40 (1H, br. d, J 4.1), 4.57-4.51 (1H, m), 3.23 (1H, tt, J 11.6 and 3.9), 3.12 (1H, dd, J 15.8 and 5.0), 3.07-2.98 (2H, m), 2.90 (1H, app. dd, J 15.8 and 1.7), 2.63 (2H, td, J 12.0 and 1.9), 2.08-1.99 (2H, m), 1.67 (2H, qd, J 12.1 and 3.6). [piperidine NH not identifiable as a distinct resonance in this solvent.]

### Example 131: N-((1S,2R)-2-hydroxy-2,3-dihydro-1H-inden-1-yl)-2-(piperidin-4-yl)benzo[d]thiazole-6-carboxamide

Prepared using (1 S,2R)-1-amino-2-indanol as the amine component.

¹H NMR (free base form): δ_{H} (400 MHz, DMSO-d₆) 8.70 (1H, app. d, J 1.6), 8.40 (1H, br. d, J 8.4), 8.07 (1H, tented dd, J 8.5 and 1.6), 7.99 (1H, tented d, J 8.5), 7.31-7.16 (4H, m), 5.47 (1H, dd, J 8.4 and 5.1), 5.40 (1H, br. d, J 4.1), 4.57-4.51 (1H, m), 3.23 (1H, tt, J 11.6 and 3.9), 3.12 (1H, dd, J 16.2 and 5.1), 3.07-2.99 (2H, m), 2.90 (1H, app. dd, J 16.2 and 1.1), 2.63 (2H, td, J 12.0 and 1.9), 2.08-1.99 (2H, m), 1.67 (2H, qd, J 12.1 and 3.6). [piperidine NH not identifiable as a distinct resonance in this solvent.]

### Example 132: (S)-N-(chroman-4-yl)-2-(piperidin-4-yl)benzo[d]thiazole-6-carboxamide

Prepared using (S)-chroman-4-amine as the amine component.

¹H NMR (hydrochloride salt form): δ_{H} (400 MHz, DMSO-d₆) 9.07-8.93 (1H, br. m), 8.99 (1H, br. d, J 8.1), 8.88-8.72 (1H, br. m), 8.66 (1H, dd, J 1.6 and 0.6), 8.05 (1H, tented dd, J 8.5 and 1.6), 8.02 (1H, tented dd, J 8.5 and 0.6), 7.22-7.14 (2H, m), 6.88 (1H, td, J7.5 and 1.1), 6.81 (1H, dd, J 8.2 and 1.1), 5.33-5.28 (1H, m), 4.35-4.22 (2H, m), 3.23 (1H, tt, J 11.3 and 3.7), 3.52-3.38 (2H, m), 3.13-3.01 (2H, m), 2.32-2.24 (2H, m), 2.20-1.96 (4H, m).

### Example 133: N-isopropyl-2-(piperidin-4-yl)benzo[d]thiazole-6-carboxamide

Prepared using isopropylamine as the amine component.

¹H NMR (free base form): δ_{H} (400 MHz, DMSO-d₆) 8.53 (1H, dd, J 1.6 and 0.5), 8.32 (1H, br. d, J7.7), 7.98 (1H, tented dd, J 8.5 and 0.5), 7.95 (1H, tented dd, J 8.5 and 1.6), 4.18-4.06 (1H, m), 3.22 (1H, tt, J 11.6 and 3.9), 3.06-2.99 (2H, m), 2.62 (2H, td, J 11.8 and 1.9), 2.06-1.99 (2H, m), 1.66 (2H, qd, J 12.2 and 3.6), 1.18 (6H, d, J6.6). [piperidine NH not identifiable as a distinct resonance in this solvent.]

### Example 134: N-cyclopentyl-2-(piperidin-4-yl)benzo[d]thiazole-6-carboxamide

Prepared using cyclopentylamine as the amine component.

¹H NMR (hydrochloride salt form): δ_{H} (400 MHz, DMSO-d₆) 9.35-9.17 (1H, br. m), 9.17-8.96 (1H, br. m), 8.59 (1H, app. d, J 1.5), 8.45 (1H, br. d, J 7.2), 8.01 (1H, tented d, J 8.5), 7.98 (1H, tented dd, J 8.5 and 1.5), 4.29-4.20 (1H, m), 3.53 (1H, tt, J 11.2 and 3.6), 3.40-3.32 (2H, m), 3.12-3.00 (2H, m), 2.32-2.23 (2H, m), 2.13-1.97 (2H, m), 1.95-1.81 (2H, m), 1.77-1.64 (2H, m), 1.64-1.45 (4H, m).

### Example 135: N-cyclohexyl-2-(piperidin-4-yl)benzo[d]thiazole-6-carboxamide

Prepared using cyclohexylamine as the amine component.

¹H NMR (free base form): δ_{H} (400 MHz, DMSO-d₆) 8.53 (1H, dd, J 1.6 and 0.5), 8.32 (1H, br. d, J 7.8), 7.98 (1H, tented dd, J 8.5 and 0.5), 7.94 (1H, tented dd, J 8.5 and 1.6), 3.84-3.71 (1H, m), 3.22 (1H, tt, J 11.5 and 3.6), 3.06-2.99 (2H, m), 2.62 (2H, td, J 11.8 and 1.9), 2.06-1.99 (2H, m), 1.90-1.79 (2H, m), 1.79-1.69 (2H, m), 1.66 (2H, qd, J 12.2 and 3.6), 1.65-1.57 (1H, m), 1.39-1.23 (4H, m), 1.21-1.07 (1H, m). [piperidine NH not identifiable as a distinct resonance in this solvent.]

### Example 136: N-(4,4-difluorocyclohexyl)-2-(piperidin-4-yl)benzo[d]thiazole-6-carboxamide

Prepared using 4,4-difluorocyclohexylamine as the amine component.

¹H NMR (free base form): δ_{H} (400 MHz, DMSO-d₆) 8.55 (1H, app. d, J 1.6), 8.41 (1H, br. d, J 7.4), 7.99 (1H, tented d, J 8.5), 7.95 (1H, tented dd, J 8.5 and 1.6), 4.09-3.96 (1H, m), 3.22 (1H, tt, J 11.5 and 3.6), 3.06-2.99 (2H, m), 2.62 (2H, td, J 11.8 and 1.9), 2.14-1.85 (8H, m), 1.74-1.59 (4H, m). [piperidine NH not identifiable as a distinct resonance in this solvent.]

### Example 137: N-(3,3-difluorocyclobutyl)-2-(piperidin-4-yl)benzo[d]thiazole-6-carboxamide

Prepared using 3,3-difluorocyclobutylamine as the amine component.

¹H NMR (free base form): δ_{H} (400 MHz, DMSO-d₆) 8.96 (1H, br. d, J6.5), 8.56 (1H, app. d, J 1.6), 8.02 (1H, tented d, J 8.6), 7.96 (1H, tented dd, J 8.6 and 1.6), 4.46-4.22 (1H, m), 3.22 (1H, tt, J 11.6 and 3.6), 3.07-2.91 (4H, m), 2.85-2.69 (2H, m), 2.62 (2H, td, J 11.8 and 1.9), 2.06-1.99 (2H, m), 1.66 (2H, qd, J 12.2 and 3.6). [piperidine NH not identifiable as a distinct resonance in this solvent.]

### Example 138: 2-(piperidin-4-yl)-N-(tetrahydro-2H-pyran-4-yl)benzo[d]thiazole-6-carboxamide

Prepared using 4-aminotetrahydropyran as the amine component.

¹H NMR (free base form): δ_{H} (400 MHz, DMSO-d₆) 8.55 (1H, dd, J 1.6 and 0.5), 8.42 (1H, br. d, J 7.6), 7.99 (1H, tented dd, J 8.6 and 0.5), 7.95 (1H, tented dd, J 8.6 and 1.6), 4.07-3.97 (1H, m), 3.92-3.85 (2H, m), 3.39 (2H, td, J 11.8 and 2.0), 3.22 (1H, tt, J 11.6 and 3.7), 3.07-2.99 (2H, m), 2.62 (2H, td, J 11.8 and 1.9), 2.07-1.98 (2H, m), 1.81-1.74 (2H, m), 1.66 (2H, qd, J 12.2 and 3.5), 1.59 (2H, qd, J 11.8 and 4.0). [piperidine NH not identifiable as a distinct resonance in this solvent.]

### Example 139: (S)-N-(2-methoxy-1-phenylethyl)-2-(piperidin-4-yl)benzo[d]thiazole-6-carboxamide

Prepared using (S)-2-methoxy-1-phenylethylamine as the amine component.

¹H NMR (free base form): δ_{H} (400 MHz, DMSO-d₆) 8.96 (1H, br. d, J8.0), 8.60 (1H, app. s), 8.03-7.98 (2H, m), 7.44 (2H, tented d, J 7.5), 7.34 (2H, t, J 7.5), 7.25 (1H, tented t, J 7.5), 5.33-5.27 (1H, m), 3.71 (1H, app. t, J9.4), 3.56 (1H, dd, J 10.0 and 5.4), 3.30 (3H, s), 3.22 (1H, tt, J 11.6 and 3.6), 3.06-2.99 (2H, m), 2.62 (2H, td, J 11.8 and 1.9), 2.07-1.98 (2H, m), 1.66 (2H, qd, J 12.2 and 3.6). [piperidine NH not identifiable as a distinct resonance in this solvent.]

### Example 140: (R)-N-(2-methoxy-1-phenylethyl)-2-(piperidin-4-yl)benzo[d]thiazole-6-carboxamide

Prepared using (R)-2-methoxy-1-phenylethylamine as the amine component.

¹H NMR (free base form): δ_{H} (400 MHz, DMSO-d₆) 8.96 (1H, br. d, J8.2), 8.60 (1H, app. s), 8.03-7.98 (2H, m), 7.44 (2H, tented d, J 7.5), 7.34 (2H, t, J 7.5), 7.26 (1H, tented t, J 7.5), 5.33-5.27 (1H, m), 3.71 (1H, dd, J10.0 and 8.8), 3.56 (1H, dd, J 10.0 and 5.4), 3.30 (3H, s), 3.22 (1H, tt, J 11.6 and 3.6), 3.06-2.99 (2H, m), 2.62 (2H, td, J 11.8 and 1.9), 2.07-1.98 (2H, m), 1.66 (2H, qd, J 12.2 and 3.6). [piperidine NH not identifiable as a distinct resonance in this solvent.]

### Example 141: (R)-N-(2-hydroxy-1-phenylethyl)-2-(piperidin-4-yl)benzo[d]thiazole-6-carboxamide

Prepared using (R)-2-hydroxy-1-phenylethylamine as the amine component.

¹H NMR (free base form): δ_{H} (400 MHz, DMSO-d₆) 8.85 (1H, br. d, J7.7), 8.62 (1H, app. s), 8.00 (2H, tight ABm/app. s), 7.41 (2H, tented d, J7.5), 7.32 (2H, t, J7.5), 7.23 (1H, tented t, J7.5), 5.12-5.06 (1H, m), 5.01 (1H, br. t, J5.3), 3.77-3.61 (2H, m), 3.22 (1H, tt, J11.6 and 3.6), 3.06-2.99 (2H, m), 2.62 (2H, td, J 11.8 and 1.9), 2.07-1.98 (2H, m), 1.66 (2H, qd, J 12.2 and 3.6). [piperidine NH not identifiable as a distinct resonance in this solvent.]

### Examples 142 to 161

Examples 142 to 161 may be prepared according to the route shown in Scheme 10.

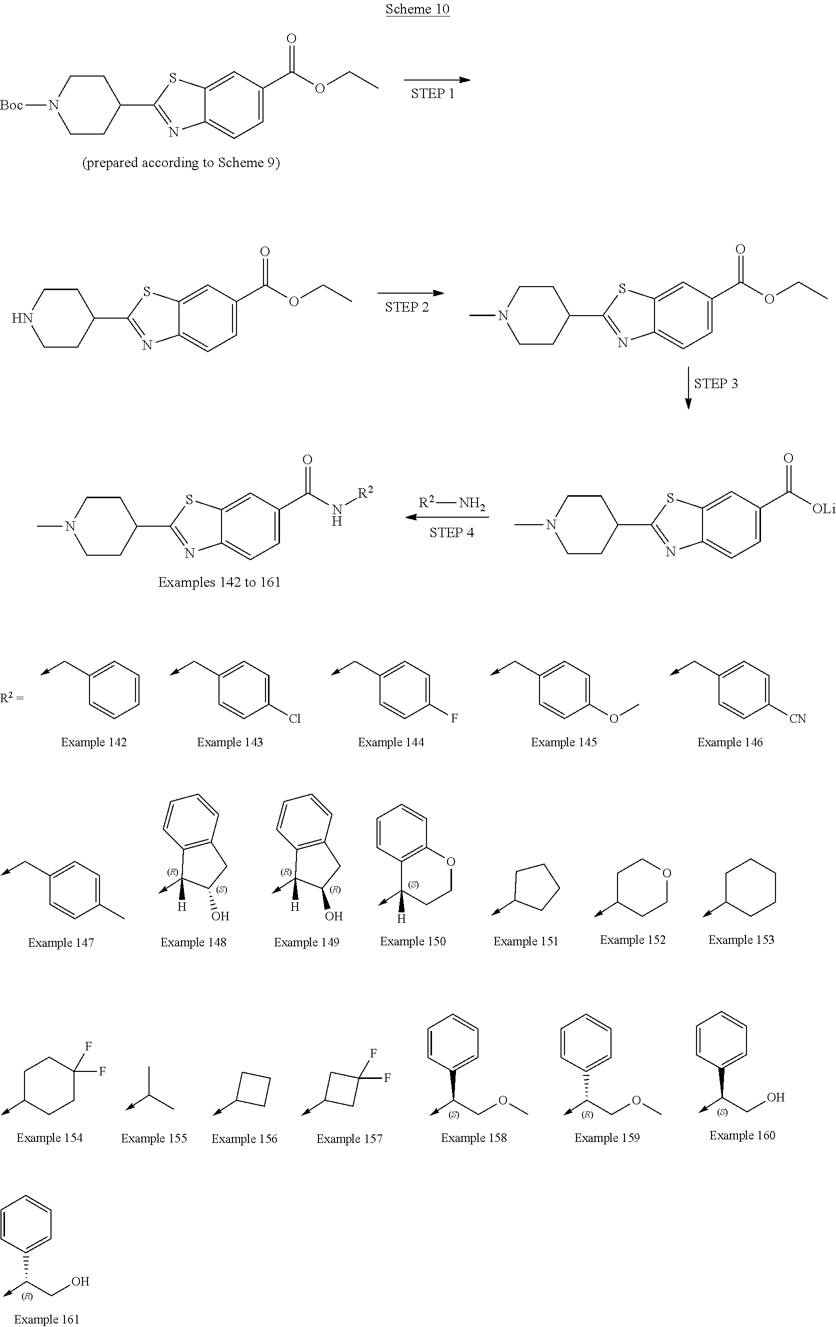

### Step 1 (Scheme 10): Synthesis of ethyl 2-(piperidin-4-yl)benzo[d]thiazole-6-carboxylate

To a solution of ethyl 2-(1-(tert-butoxycarbonyl)piperidin-4-yl)benzo[d]thiazole-6-carboxylate (1.0 equiv.; prepared as described in Scheme 9 for Examples 123-141) in DCM (0.34 M in substrate) at 0° C. was added trifluoroacetic acid (3.0 equiv.). The mixture was stirred at ambient temperature for 3 h and then washed with aqueous base, dried (Na₂SO₄) and evaporated to dryness at reduced pressure. The residue was triturated with pentane and dried to obtain ethyl 2-(piperidin-4-yl)benzo[d]thiazole-6-carboxylate as brown solid (94% yield). The product was used in the next step without further purification.

### Step 2 (Scheme 10): Synthesis of ethyl 2-(1-methylpiperidin-4-yl)benzo[d]thiazole-6-carboxylate

To a stirred solution of ethyl 2-(piperidin-4-yl)benzo[d]thiazole-6-carboxylate (1.0 equiv.) in MeOH (0.33 M in substrate) was added formaldehyde (37% w/w in H₂O; 1.0 equiv.) and acetic acid (0.1 equiv.). The mixture was stirred at ambient temperature for 3 h and then cooled to 0° C. prior to addition of sodium cyanoborohydride (2.0 equiv.), stirring for a further period of 1 h (monitored by TLC). The reaction mixture was quenched with ice-cold water, concentrated under reduced pressure and extracted with DCM. The DCM extract was washed with saturated NaHCO₃ solution followed by brine, dried (Na₂SO₄) and evaporated to dryness under reduced pressure to afford ethyl 2-(1-methylpiperidin-4-yl)benzo[d]thiazole-6-carboxylate as a yellow solid (93% yield). The product was used in the next step without further purification.

### Step 3 (Scheme 10): Synthesis of 2-(1-methylpiperidin-4-yl)benzo[d]thiazole-6-carboxylic acid lithium salt

To a stirred mixture of ethyl 2-(1-methylpiperidin-4-yl)benzo[d]thiazole-6-carboxylate (1.0 equiv.) in 3/3/1 v/v/v THF/MeOH/H₂O (0.17 M in substrate) was added LiOH·H₂O (2.0 equiv.). After 16 h the mixture was evaporated under reduced pressure and the residue triturated with hexanes and dried to obtain 2-(1-methylpiperidin-4-yl)benzo[d]thiazole-6-carboxylic acid lithium salt as brown solid that was used directly in the next step.

### Step 4 (Scheme 10) leading to Examples 142 to 161: General procedure for amide coupling

To a stirred mixture of 2-(1-methylpiperidin-4-yl)benzo[d]thiazole-6-carboxylic acid lithium salt (1.0 equiv.) in DCM (0.05 M in substrate) at 0° C. was added the required amine component (1.1 equiv.), n-propylphosphonic acid anhydride cyclic trimer (50% in EtOAc; 1.0 equiv.) and DIPEA (4.0 equiv.). The reaction mixture was brought to ambient temperature, stirring for 16 h, and then diluted with DCM and washed with water followed by brine. The organic phase was dried (Na₂SO₄) and evaporated to afford a residue that was processed by flash chromatography (1-10% MeOH in DCM eluent). Fractions containing the product were combined and evaporated to afford the target amide derivative as a solid.

### Example 142: N-benzyl-2-(1-methylpiperidin-4-yl)benzo[d]thiazole-6-carboxamide

Prepared using benzylamine as the amine component.

¹H NMR (free base form): δ_{H} (400 MHz, DMSO-d₆) 9.14 (1H, br. t, J 5.9), 8.60 (1H, app. s), 8.03-7.98 (2H, m), 7.37-7.21 (5H, m), 4.51 (2H, d, J5.9), 3.10 (1H, tt, J 11.4 and 3.7), 2.90-2.81 (2H, m), 2.20 (3H, s), 2.14-2.00 (4H, m), 1.88-1.75 (2H, m).

### Example 143: N-(4-chlorobenzyl)-2-(1-methylpiperidin-4-yl)benzo[d]thiazole-6-carboxamide

Prepared using 4-chlorobenzylamine as the amine component.

¹H NMR (free base form): δ_{H} (400 MHz, DMSO-d₆) 9.17 (1H, br. t, J5.9), 8.60 (1H, dd, J 1.6 and 0.6), 8.02 (1H, tented dd, J 8.6 and 0.7), 7.99 (1H, tented dd, J 8.6 and 1.6), 7.41-7.35 (4H, AA′BB′ m), 4.49 (2H, d, J 5.9), 3.16 (1H, tt, J 11.4 and 3.7), 3.00-2.89 (2H, m), 2.29 (3H, s), 2.28-2.07 (4H, m), 1.92-1.79 (2H, m).

### Example 144: N-(4-fluorobenzyl)-2-(1-methylpiperidin-4-yl)benzo[d]thiazole-6-carboxamide

Prepared using 4-fluorobenzylamine as the amine component.

¹H NMR (free base form): δ_{H} (400 MHz, DMSO-d₆) 9.15 (1H, br. t, J5.9), 8.59 (1H, dd, J 1.6 and 0.6), 8.01 (1H, tented dd, J 8.6 and 0.6), 7.98 (1H, tented dd, J 8.6 and 1.6), 7.41-7.35 (2H, m), 7.19-7.12 (2H, m), 4.49 (2H, d, J 5.9), 3.10 (1H, tt, J 11.4 and 3.7), 2.90-2.81 (2H, m), 2.20 (3H, s), 2.14-2.00 (4H, m), 1.88-1.75 (2H, m).

### Example 145: N-(4-methoxybenzyl)-2-(1-methylpiperidin-4-yl)benzo[d]thiazole-6-carboxamide

Prepared using 4-methoxybenzylamine as the amine component.

¹H NMR (free base form): δ_{H} (400 MHz, DMSO-d₆) 9.05 (1H, br. t, J5.9), 8.58 (1H, dd, J 1.5 and 0.8), 8.00 (1H, tented dd, J 8.6 and 0.8), 7.98 (1H, tented dd, J 8.6 and 1.5), 7.29-7.25 (2H, AA′BB′ m), 6.91-6.87 (2H, AA′BB′ m), 4.43 (2H, d, J 5.9), 3.73 (3H, s), 3.10 (1H, tt, J 11.4 and 3.7), 2.90-2.81 (2H, m), 2.20 (3H, s), 2.14-2.00 (4H, m), 1.88-1.75 (2H, m).

### Example 146: N-(4-cyanobenzyl)-2-(1-methylpiperidin-4-yl)benzo[d]thiazole-6-carboxamide

Prepared using 4-cyanobenzylamine as the amine component.

¹H NMR (free base form): δ_{H} (400 MHz, DMSO-d₆) 9.26 (1H, br. t, J5.9), 8.61 (1H, dd, J 1.6 and 0.6), 8.03 (1H, tented dd, J 8.6 and 0.6), 7.99 (1H, tented dd, J 8.6 and 1.6), 7.83-7.79 (2H, AA′BB′ m), 7.56-7.51 (2H, AA′BB′ m), 4.58 (2H, d, J 5.9), 3.11 (1H, tt, J 11.4 and 3.7), 2.90-2.81 (2H, m), 2.22 (3H, s), 2.14-2.00 (4H, m), 1.88-1.75 (2H, m).

### Example 147: N-(4-methylbenzyl)-2-(1-methylpiperidin-4-yl)benzo[d]thiazole-6-carboxamide

Prepared using 4-methylbenzylamine as the amine component.

¹H NMR (free base form): δ_{H} (400 MHz, DMSO-d₆) 9.08 (1H, br. t, J5.9), 8.58 (1H, dd, J 1.5 and 0.8), 8.01 (1H, tented dd, J 8.6 and 0.8), 7.98 (1H, tented dd, J 8.6 and 1.5), 7.25-7.20 (2H, AA′BB′ m), 7.16-7.11 (2H, AA′BB′ m), 4.46 (2H, d, J 5.9), 3.10 (1H, tt, J 11.4 and 3.7), 2.90-2.81 (2H, m), 2.27 (3H, s), 2.22 (3H, s), 2.14-2.00 (4H, m), 1.88-1.75 (2H, m).

### Example 148: N-((1R,2S)-2-hydroxy-2,3-dihydro-1H-inden-1-yl)-2-(1-methylpiperidin-4-yl)benzo[d]thiazole-6-carboxamide

Prepared using (1R,2S)-1-amino-2-indanol as the amine component.

¹H NMR (free base form): δ_{H} (400 MHz, DMSO-d₆) 8.70 (1H, app. d, J 1.6), 8.38 (1H, br. d, J 8.5), 8.07 (1H, tented dd, J 8.5 and 1.6), 8.00 (1H, tented d, J 8.5), 7.30-7.16 (4H, m), 5.47 (1H, dd, J 8.5 and 5.2), 5.12 (1H, br. d, J 4.5), 4.56-4.52 (1H, m), 3.16-3.07 (2H, m), 2.93-2.82 (3H, m), 2.21 (3H, s), 2.14-2.00 (4H, m), 1.88-1.75 (2H, m).

### Example 149: N-((1R,2R)-2-hydroxy-2,3-dihydro-1H-inden-1-yl)-2-(1-methylpiperidin-4-yl)benzo[d]thiazole-6-carboxamide

Prepared using (1R,2R)-1-amino-2-indanol as the amine component.

¹H NMR (free base form): δ_{H} (400 MHz, DMSO-d₆) 8.85 (1H, br. d, J 8.4), 8.64 (1H, app. d, J 1.6), 8.04 (1H, tented dd, J 8.5 and 1.6), 8.01 (1H, tented d, J 8.5), 7.24-7.11 (4H, m), 5.37 (1H, br. d, J5.7), 5.34-5.31 (1H, m), 4.48-4.41 (1H, m), 3.19 (1H, dd, J15.5 and 7.2), 3.11 (1H, tt, J 11.4 and 3.7), 2.90-2.82 (2H, m), 2.76 (1H, dd, J 15.5 and 7.7), 2.20 (3H, s), 2.14-2.00 (4H, m), 1.88-1.75 (2H, m).

### Example 150: (S)-N-(chroman-4-yl)-2-(1-methylpiperidin-4-yl)benzo[d]thiazole-6-carboxamide

Prepared using (S)-chroman-4-amine as the amine component.

¹H NMR (free base form): δ_{H} (400 MHz, DMSO-d₆) 8.98 (1H, br. d, J8.1), 8.62 (1H, dd, J 1.6 and 0.5), 8.03 (1H, tented dd, J 8.5 and 1.6), 7.99 (1H, tented dd, J 8.5 and 0.5), 7.20 (1H, app. d, J7.6), 7.16 (1H, app. td, J7.7 and 1.5), 6.88 (1H, td, J7.5 and 1.1), 6.81 (1H, dd, J 8.2 and 1.1), 5.33-5.28 (1H, m), 4.34-4.22 (2H, m), 3.10 (1H, tt, J 11.4 and 3.7), 2.90-2.82 (2H, m), 2.20 (3H, s), 2.18-2.00 (6H, m), 1.88-1.75 (2H, m).

### Example 151: N-cyclopentyl-2-(1-methylpiperidin-4-yl)benzo[d]thiazole-6-carboxamide

Prepared using cyclopentylamine as the amine component.

¹H NMR (free base form): δ_{H} (400 MHz, DMSO-d₆) 8.54 (1H, app. d, J 1.6), 8.38 (1H, br. d, J 7.2), 7.98 (1H, tented d, J 8.5), 7.95 (1H, tented dd, J 8.5 and 1.6), 4.30-4.20 (1H, m), 3.10 (1H, tt, J 11.4 and 3.7), 2.90-2.81 (2H, m), 2.20 (3H, s), 2.14-2.00 (4H, m), 1.96-1.65 (6H, m), 1.88-1.75 (4H, m).

### Example 152: 2-(1-methylpiperidin-4-yl)-N-(tetrahydro-2H-pyran-4-yl)benzo[d]thiazole-6-carboxamide

Prepared using 4-aminotetrahydropyran as the amine component.

¹H NMR (free base form): δ_{H} (400 MHz, DMSO-d₆) 8.55 (1H, dd, J 1.6 and 0.5), 8.41 (1H, br. d, J 7.7), 8.00 (1H, tented dd, J 8.6 and 0.5), 7.96 (1H, tented dd, J 8.6 and 1.6), 4.08-3.97 (1H, m), 3.92-3.85 (2H, m), 3.40 (2H, td, J 11.8 and 2.0), 3.10 (1H, tt, J 11.4 and 3.7), 2.89-2.81 (2H, m), 2.20 (3H, s), 2.14-2.00 (4H, m), 1.88-1.73 (4H, m), 1.59 (2H, app. qd, J 11.8 and 4.0).

### Example 153: N-cyclohexyl-2-(1-methylpiperidin-4-yl)benzo[d]thiazole-6-carboxamide

Prepared using cyclohexylamine as the amine component.

¹H NMR (free base form): δ_{H} (400 MHz, DMSO-d₆) 8.53 (1H, dd, J 1.6 and 0.5), 8.31 (1H, br. d, J 7.8), 7.98 (1H, tented dd, J 8.5 and 0.5), 7.94 (1H, tented dd, J 8.5 and 1.6), 3.84-3.71 (1H, m), 3.10 (1H, tt, J 11.4 and 3.7), 2.89-2.81 (2H, m), 2.20 (3H, s), 2.13-2.00 (4H, m), 1.88-1.69 (6H, m), 1.65-1.58 (1H, m), 1.39-1.22 (4H, m), 1.20-1.07 (1H, m).

### Example 154: N-(4,4-difluorocyclohexyl)-2-(1-methylpiperidin-4-yl)benzo[d]thiazole-6-carboxamide

Prepared using 4,4-difluorocyclohexylamine as the amine component.

¹H NMR (free base form): δ_{H} (400 MHz, DMSO-d₆) 8.55 (1H, app. d, J 1.6), 8.41 (1H, br. d, J 7.6), 8.00 (1H, tented d, J 8.5), 7.95 (1H, tented dd, J 8.5 and 1.6), 4.07-3.96 (1H, m), 3.10 (1H, tt, J 11.4 and 3.7), 2.89-2.81 (2H, m), 2.20 (3H, s), 2.13-1.75 (12H, m), 1.72-1.59 (2H, m).

### Example 155: N-isopropyl-2-(1-methylpiperidin-4-yl)benzo[d]thiazole-6-carboxamide

Prepared using isopropylamine as the amine component.

¹H NMR (free base form): δ_{H} (400 MHz, DMSO-d₆) 8.54 (1H, dd, J 1.6 and 0.5), 8.32 (1H, br. d, J7.7), 7.99 (1H, tented dd, J 8.5 and 0.5), 7.95 (1H, tented dd, J 8.5 and 1.6), 4.18-4.05 (1H, m), 3.10 (1H, tt, J 11.4 and 3.7), 2.89-2.81 (2H, m), 2.20 (3H, s), 2.13-2.00 (4H, m), 1.88-1.75 (2H, m), 1.18 (6H, d, J 6.6).

### Example 156: N-cyclobutyl-2-(1-methylpiperidin-4-yl)benzo[d]thiazole-6-carboxamide

Prepared using cyclobutylamine as the amine component.

¹H NMR (free base form): δ_{H} (400 MHz, DMSO-d₆) 8.72 (1H, br. d, J7.5), 8.54 (1H, dd, J 1.6 and 0.5), 7.99 (1H, tented dd, J 8.5 and 0.5), 7.95 (1H, tented dd, J 8.5 and 1.6), 4.49-4.39 (1H, m), 3.10 (1H, tt, J 11.4 and 3.7), 2.89-2.81 (2H, m), 2.27-2.20 (2H, m), 2.20 (3H, s), 2.14-2.00 (6H, m), 1.88-1.75 (2H, m), 1.74-1.62 (2H, m).

### Example 157: N-(3,3-difluorocyclobutyl)-2-(1-methylpiperidin-4-yl)benzo[d]thiazole-6-carboxamide

Prepared using 3,3-difluorocyclobutylamine as the amine component.

¹H NMR (free base form): δ_{H} (400 MHz, DMSO-d₆) 8.93 (1H, br. d, J6.6), 8.56 (1H, app. d, J 1.7), 8.02 (1H, tented d, J 8.6), 7.96 (1H, tented dd, J 8.6 and 1.7), 4.36-4.24 (1H, m), 3.11 (1H, tt, J 11.4 and 3.7), 3.02-2.91 (2H, m), 2.89-2.70 (4H, m), 2.20 (3H, s), 2.13-2.00 (4H, m), 1.88-1.75 (2H, m).

### Example 158: (S)-N-(2-methoxy-1-phenylethyl)-2-(1-methylpiperidin-4-yl)benzo[d]thiazole-6-carboxamide

Prepared using (S)-2-methoxy-1-phenylethylamine as the amine component.

¹H NMR (free base form): δ_{H} (400 MHz, DMSO-d₆) 8.94 (1H, br. d, J 8.2), 8.60 (1H, dd, J 1.6 and 0.6), 8.02 (1H, tented dd, J 8.6 and 0.6), 8.00 (1H, tented dd, J 8.6 and 1.6), 7.44 (2H, tented app. d, J 7.5), 7.34 (2H, t, J 7.5), 7.25 (1H, tented app. t, J 7.5), 5.33-5.27 (1H, m), 3.71 (1H, dd, J 10.0 and 8.9), 3.57 (1H, dd, J 10.0 and 5.3), 3.30 (3H, s), 3.11 (1H, tt, J 11.4 and 3.7), 2.89-2.81 (2H, m), 2.20 (3H, s), 2.13-2.00 (4H, m), 1.88-1.75 (2H, m).

### Example 159: (R)-N-(2-methoxy-1-phenylethyl)-2-(1-methylpiperidin-4-yl)benzo[d]thiazole-6-carboxamide

Prepared using (R)-2-methoxy-1-phenylethylamine as the amine component.

¹H NMR (free base form): δ_{H} (400 MHz, DMSO-d₆) 8.94 (1H, br. d, J 8.2), 8.60 (1H, dd, J 1.6 and 0.6), 8.02 (1H, tented dd, J 8.6 and 0.6), 8.00 (1H, tented dd, J 8.6 and 1.6), 7.44 (2H, tented app. d, J 7.5), 7.34 (2H, t, J 7.5), 7.25 (1H, tented app. t, J 7.5), 5.33-5.27 (1H, m), 3.71 (1H, dd, J 10.0 and 8.9), 3.57 (1H, dd, J 10.0 and 5.3), 3.30 (3H, s), 3.11 (1H, tt, J 11.4 and 3.7), 2.89-2.81 (2H, m), 2.20 (3H, s), 2.13-2.00 (4H, m), 1.88-1.75 (2H, m).

### Example 160: (S)-N-(2-hydroxy-1-phenylethyl)-2-(1-methylpiperidin-4-yl)benzo[d]thiazole-6-carboxamide

Prepared using (S)-2-hydroxy-1-phenylethylamine as the amine component.

¹H NMR (free base form): δ_{H} (400 MHz, DMSO-d₆) 8.80 (1H, br. d, J8.2), 8.62 (1H, app. s), 8.01 (2H, tight ABm/app. s), 7.41 (2H, tented app. d, J7.5), 7.32 (2H, t, J7.5), 7.23 (1H, tented app. t, J7.5), 5.13-5.07 (1H, m), 4.94 (1H, br. t, J 5.9), 3.77-3.63 (2H, m), 3.11 (1H, tt, J 11.4 and 3.7), 2.89-2.81 (2H, m), 2.20 (3H, s), 2.13-2.00 (4H, m), 1.88-1.75 (2H, m).

### Example 161: (R)-N-(2-hydroxy-1-phenylethyl)-2-(1-methylpiperidin-4-yl)benzo[d]thiazole-6-carboxamide

Prepared using (R)-2-hydroxy-1-phenylethylamine as the amine component.

¹H NMR (free base form): δ_{H} (400 MHz, DMSO-d₆) 8.82 (1H, br. d, J8.1), 8.62 (1H, app. s), 8.01 (2H, tight ABm/app. s), 7.41 (2H, tented app. d, J7.5), 7.32 (2H, t, J7.5), 7.23 (1H, tented app. t, J7.5), 5.12-5.07 (1H, m), 4.96 (1H, br. t, J 5.9), 3.77-3.63 (2H, m), 3.11 (1H, tt, J 11.4 and 3.7), 2.89-2.81 (2H, m), 2.20 (3H, s), 2.13-2.00 (4H, m), 1.88-1.75 (2H, m).

### Examples 162 to 168

Examples 162 to 168 may be prepared according to the route shown in Scheme 11.

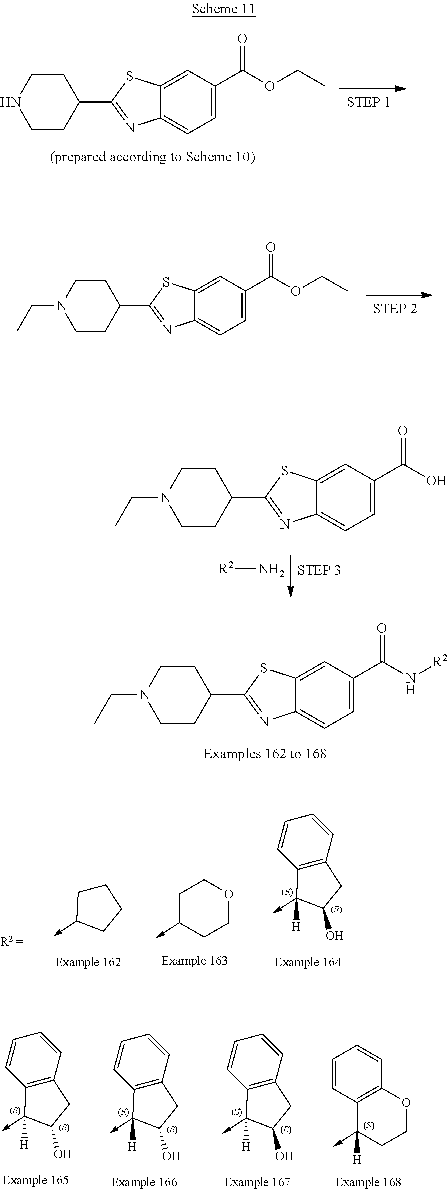

### Step 1 (Scheme 11): Synthesis of ethyl 2-(1-ethylpiperidin-4-yl)benzo[d]thiazole-6-carboxylate

To a stirred solution of ethyl 2-(piperidin-4-yl)benzo[d]thiazole-6-carboxylate (1.0 equiv.; prepared as described in Scheme 10 for Examples 142-161) in MeOH (0.23 M in substrate) was added acetaldehyde (2.0 equiv.) and acetic acid (0.1 equiv.). The mixture was stirred at ambient temperature for 3 h and then cooled to 0° C. prior to addition of sodium cyanoborohydride (2.0 equiv.), stirring for a further period of 1 h (monitored by TLC). The reaction mixture was quenched with ice-cold water, concentrated under reduced pressure and extracted with DCM. The DCM extract was washed with saturated NaHCO₃ solution followed by brine, dried (Na₂SO₄) and evaporated to dryness under reduced pressure to afford ethyl 2-(1-ethylpiperidin-4-yl)benzo[d]thiazole-6-carboxylate as a yellow solid (82% yield). The product was used in the next step without further purification.

### Step 2 (Scheme 11): Synthesis of 2-(1-ethylpiperidin-4-yl)benzo[d]thiazole-6-carboxylic acid

To a stirred mixture of ethyl 2-(1-ethylpiperidin-4-yl)benzo[d]thiazole-6-carboxylate (1.0 equiv.) in 2.3/1 v/v THF/H₂O (0.03 M in substrate) was added LiOH·H₂O (3.0 equiv.). The reaction mixture was stirred at ambient temperature for 16 h (monitored by TLC) and then evaporated. The residue was diluted with ice-cold water and acidified with 1.5 N hydrochloric acid to precipitate a solid that was collected by filtration, washing with water, and dried in vacuo to afford 2-(1-ethylpiperidin-4-yl)benzo[d]thiazole-6-carboxylic acid as a pale yellow solid. The product was used in the next step without further purification.

### Step 3 (Scheme 11) leading to Examples 162 to 168: General procedure for amide coupling

To a stirred mixture of 2-(1-ethylpiperidin-4-yl)benzo[d]thiazole-6-carboxylic acid (1.0 equiv.) in DCM (0.05 M in substrate) at 0° C. was added the required amine component (1.1 equiv.), n-propylphosphonic acid anhydride cyclic trimer (50% in EtOAc; 1.0 equiv.) and DIPEA (4.0 equiv.). The reaction mixture was brought to ambient temperature, stirring for 16 h, and then diluted with DCM and washed with water followed by brine. The organic phase was dried (Na₂SO₄) and evaporated to afford a residue that was processed by flash column chromatography (1-10% MeOH/DCM). Fractions containing the product were combined and evaporated to afford the target amide derivative as a solid.

### Example 162: N-cyclopentyl-2-(1-ethylpiperidin-4-yl)benzo[d]thiazole-6-carboxamide

Prepared using cyclopentylamine as the amine component.

¹H NMR (free base form): δ_{H} (400 MHz, DMSO-d₆) 8.54 (1H, dd, J 1.6 and 0.5), 8.37 (1H, br. d, J 7.2), 7.98 (1H, tented dd, J 8.5 and 0.5), 7.95 (1H, tented dd, J 8.5 and 1.6), 4.30-4.20 (1H, m), 3.12 (1H, tt, J11.4 and 3.8), 3.00-2.90 (2H, m), 2.36 (2H, q, J7.2), 2.14-2.00 (4H, m), 1.95-1.65 (6H, m), 1.60-1.48 (4H, m), 1.01 (3H, t, J 7.2).

### Example 163: 2-(1-ethylpiperidin-4-yl)-N-(tetrahydro-2H-pyran-4-yl)benzo[d]thiazole-6-carboxamide

Prepared using 4-aminotetrahydropyran as the amine component.

¹H NMR (free base form): δ_{H} (400 MHz, DMSO-d₆) 8.55 (1H, app. d, J 1.6), 8.43 (1H, br. d, J 7.6), 8.00 (1H, tented d, J 8.5), 7.96 (1H, tented dd, J 8.5 and 1.6), 4.08-3.97 (1H, m), 3.92-3.85 (2H, m), 3.39 (2H, td, J 11.8 and 2.0), 3.13 (1H, tt, J 11.5 and 3.8), 3.00-2.91 (2H, m), 2.35 (2H, q, J 7.2), 2.15-2.00 (4H, m), 1.89-1.73 (4H, m), 1.59 (2H, app. qd, J 11.8 and 4.0), 1.02 (3H, t, J7.2).

### Example 164: 2-(1-ethylpiperidin-4-yl)-N-((1R,2R)-2-hydroxy-2,3-dihydro-1H-inden-1-yl)benzo[d]thiazole-6-carboxamide

Prepared using (1R,2R)-1-amino-2-indanol as the amine component.

¹H NMR (free base form): δ_{H} (400 MHz, DMSO-d₆) 8.85 (1H, br. d, J 8.5), 8.64 (1H, app. d, J 1.6), 8.05 (1H, tented dd, J 8.5 and 1.6), 8.01 (1H, tented d, J 8.5), 7.25-7.11 (4H, m), 5.38 (1H, br. d, J5.6), 5.34-5.31 (1H, m), 4.48-4.41 (1H, m), 3.19 (1H, dd, J15.5 and 7.2), 3.14 (1H, tt, J 11.4 and 3.7), 3.01-2.92 (2H, m), 2.76 (1H, dd, J 15.5 and 7.6), 2.37 (2H, q, J7.2), 2.16-2.01 (4H, m), 1.87-1.74 (2H, m), 1.02 (3H, t, J7.2).

### Example 165: 2-(1-ethylpiperidin-4-yl)-N-((1S,2S)-2-hydroxy-2,3-dihydro-1H-inden-1-yl)benzo[d]thiazole-6-carboxamide

Prepared using (1S,2S)-1-amino-2-indanol as the amine component.

¹H NMR (free base form): δ_{H} (400 MHz, DMSO-d₆) 8.86 (1H, br. d, J8.4), 8.64 (1H, app. d, J 1.6), 8.05 (1H, tented dd, J 8.5 and 1.6), 8.01 (1H, tented d, J 8.5), 7.24-7.11 (4H, m), 5.38 (1H, br. d, J5.5), 5.34-5.31 (1H, m), 4.48-4.41 (1H, m), 3.19 (1H, dd, J15.5 and 7.2), 3.14 (1H, tt, J 11.4 and 3.7), 3.01-2.92 (2H, m), 2.76 (1H, dd, J 15.5 and 7.7), 2.36 (2H, q, J7.2), 2.16-2.01 (4H, m), 1.87-1.74 (2H, m), 1.02 (3H, t, J7.2).

### Example 166: 2-(1-ethylpiperidin-4-yl)-N-((1R,2S)-2-hydroxy-2,3-dihydro-1H-inden-1-yl)benzo[d]thiazole-6-carboxamide

Prepared using (1R,2S)-1-amino-2-indanol as the amine component.

¹H NMR (free base form): δ_{H} (400 MHz, DMSO-d₆) 8.70 (1H, app. d, J 1.6), 8.38 (1H, br. d, J 8.5), 8.07 (1H, tented dd, J 8.5 and 1.6), 8.00 (1H, tented d, J 8.5), 7.30-7.16 (4H, m), 5.48 (1H, dd, J 8.3 and 5.2), 5.13 (1H, br. d, J 4.5), 4.56-4.52 (1H, m), 3.18-3.08 (2H, m), 3.00-2.85 (3H, m), 2.36 (2H, q, J 7.2), 2.15-2.00 (4H, m), 1.87-1.74 (2H, m), 1.02 (3H, t, J 7.2).

### Example 167: 2-(1-ethylpiperidin-4-yl)-N-((1S,2R)-2-hydroxy-2,3-dihydro-1H-inden-1-yl)benzo[d]thiazole-6-carboxamide

Prepared using (1 S,2R)-1-amino-2-indanol as the amine component.

¹H NMR (free base form): δ_{H} (400 MHz, DMSO-d₆) 8.70 (1H, app. d, J 1.6), 8.41 (1H, br. d, J 8.6), 8.07 (1H, tented dd, J 8.5 and 1.6), 8.00 (1H, tented d, J 8.5), 7.30-7.16 (4H, m), 5.48 (1H, dd, J 8.3 and 5.2), 5.16 (1H, br. s), 4.56-4.52 (1H, m), 3.18-3.08 (2H, m), 3.00-2.85 (3H, m), 2.36 (2H, q, J7.2), 2.15-2.00 (4H, m), 1.87-1.74 (2H, m), 1.02 (3H, t, J7.2).

### Example 168: (S)-N-(chroman-4-yl)-2-(1-ethylpiperidin-4-yl)benzo[d]thiazole-6-carboxamide

Prepared using (S)-chroman-4-amine as the amine component.

¹H NMR (free base form): δ_{H} (400 MHz, DMSO-d₆) 8.97 (1H, br. d, J8.1), 8.62 (1H, dd, J 1.6 and 0.5), 8.03 (1H, tented dd, J 8.5 and 1.6), 7.99 (1H, tented dd, J 8.5 and 0.5), 7.20 (1H, app. d, J7.6), 7.16 (1H, app. td, J7.7 and 1.5), 6.88 (1H, td, J7.5 and 1.1), 6.81 (1H, dd, J 8.2 and 1.1), 5.33-5.28 (1H, m), 4.34-4.22 (2H, m), 3.13 (1H, tt, J 11.4 and 3.8), 3.00-2.90 (2H, m), 2.35 (2H, q, J 7.2), 2.20-1.99 (6H, m), 1.86-1.72 (2H, m), 1.01 (3H, t, J 7.2).

### Examples 169 to 174

Examples 169 to 174 may be prepared according to the route shown in Scheme 12.

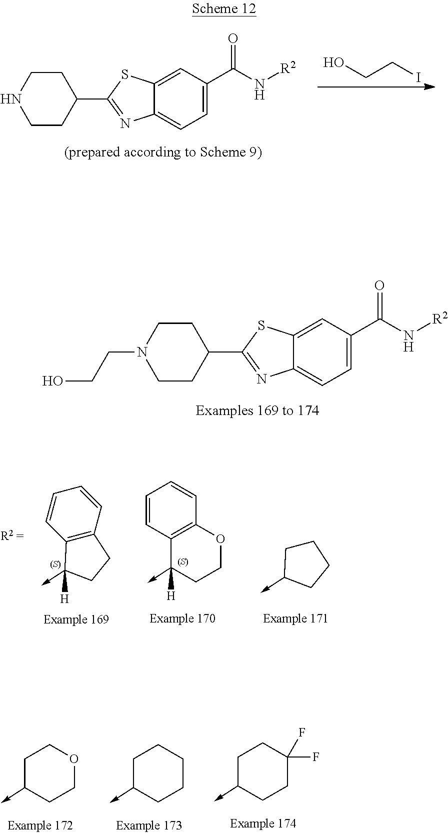

To a stirred mixture of the 2-(piperazin-1-yl)benzo[d]thiazole-6-carboxamide derivative (1.0 equiv.; prepared as described in Scheme 9) in DMF (1 volume; 0.05 M in substrate) under Ar was added K₂CO₃ (2.0 equiv.) and 2-iodoethanol (1.5 equiv.). The reaction mixture was heated to 70° C., stirring for 16 h, and then cooled, diluted with water (5 volumes) and extracted with 5% v/v MeOH/DCM (3×10 volumes). The combined organic extract was washed with brine, dried (Na₂SO₄), and concentrated in vacuo to afford the crude product. The latter was processed by preparative HPLC to afford the target 2-(1-(2-hydroxyethyl)piperidin-4-yl)benzo[d]thiazole-6-carboxamide derivative.

### Example 169: (S)-N-(2,3-dihydro-1H-inden-1-yl)-2-(1-(2-hydroxyethyl)piperidin-4-yl)benzo[d]thiazole-6-carboxamide

Prepared according to Scheme 12 starting from (S)-N-(2,3-dihydro-1H-inden-1-yl)-2-(piperidin-4-yl)benzo[d]thiazole-6-carboxamide (Example 127).

¹H NMR (free base form): δ_{H} (400 MHz, DMSO-d₆) 8.88 (1H, br. d, J8.3), 8.62 (1H, app. s), 8.04-7.98 (2H, m), 7.31-7.16 (4H, m), 5.62-5.56 (1H, m), 4.40 (1H, br. t, J 5.2), 3.51 (2H, app. q, J 5.8), 3.13 (1H, tt, J 11.5 and 3.7), 3.06-2.92 (3H, m), 2.86 (1H, tented app. dt, J 15.8 and 7.9), 2.50-2.40 (1H, m), 2.42 (2H, t, J 6.2), 2.22-1.95 (5H, m), 1.86-1.74 (2H, m).

### Example 170: (S)-N-(chroman-4-yl)-2-(1-(2-hydroxyethyl)piperidin-4-yl)benzo[d]thiazole-6-carboxamide

Prepared according to Scheme 12 starting from (S)-N-(chroman-4-yl)-2-(piperidin-4-yl)benzo[d]thiazole-6-carboxamide (Example 132).

¹H NMR (free base form): δ_{H} (400 MHz, DMSO-d₆) 8.97 (1H, br. d, J8.0), 8.62 (1H, app. s), 8.06-7.97 (2H, m), 7.20 (1H, app. d, J7.6), 7.16 (1H, app. td, J7.7 and 1.5), 6.88 (1H, td, J 7.5 and 1.1), 6.81 (1H, dd, J 8.2 and 1.1), 5.33-5.28 (1H, m), 4.39 (1H, br. t, J 5.2), 4.34-4.22 (2H, m), 3.51 (2H, app. q, J5.8), 3.12 (1H, tt, J 11.4 and 3.8), 3.00-2.90 (2H, m), 2.41 (2H, t, J6.2), 2.21-2.01 (6H, m), 1.87-1.74 (2H, m).

### Example 171: N-cyclopentyl-2-(1-(2-hydroxyethyl)piperidin-4-yl)benzo[d]thiazole-6-carboxamide

Prepared according to Scheme 12 starting from N-cyclopentyl-2-(piperidin-4-yl)benzo[d]thiazole-6-carboxamide (Example 134).

¹H NMR (free base form): δ_{H} (400 MHz, DMSO-d₆) 8.54 (1H, app. d, J 1.6), 8.39 (1H, br. d, J 7.2), 7.98 (1H, tented d, J 8.5), 7.95 (1H, tented dd, J 8.5 and 1.6), 4.41 (1H, br. s), 4.30-4.20 (1H, m), 3.57-3.48 (2H, m), 3.20-3.07 (1H, m), 3.03-2.92 (2H, m), 2.50-2.36 (2H, m), 2.26-2.03 (4H, m), 1.95-1.65 (6H, m), 1.60-1.48 (4H, m).

### Example 172: 2-(1-(2-hydroxyethyl)piperidin-4-yl)-N-(tetrahydro-2H-pyran-4-yl)benzo[d]thiazole-6-carboxamide

Prepared according to Scheme 12 starting from 2-(piperidin-4-yl)-N-(tetrahydro-2H-pyran-4-yl)benzo[d]thiazole-6-carboxamide (Example 138).

¹H NMR (free base form): δ_{H} (400 MHz, DMSO-d₆) 8.55 (1H, dd, J 1.6 and 0.5), 8.42 (1H, br. d, J7.6), 8.00 (1H, tented dd, J 8.5 and 0.5), 7.96 (1H, tented dd, J 8.5 and 1.6), 4.39 (1H, br. t, J5.3), 4.08-3.97 (1H, m), 3.92-3.85 (2H, m), 3.51 (2H, app. q, J 5.8), 3.39 (2H, td, J 11.8 and 2.0), 3.12 (1H, tt, J 11.5 and 3.8), 3.00-2.92 (2H, m), 2.42 (2H, t, J6.3), 2.20-2.05 (4H, m), 1.87-1.74 (4H, m), 1.59 (2H, app. qd, J 11.8 and 4.0).

### Example 173: N-cyclohexyl-2-(1-(2-hydroxyethyl)piperidin-4-yl)benzo[d]thiazole-6-carboxamide

Prepared according to Scheme 12 starting from N-cyclohexyl-2-(piperidin-4-yl)benzo[d]thiazole-6-carboxamide (Example 135).

¹H NMR (free base form): δ_{H} (400 MHz, DMSO-d₆) 8.53 (1H, app. d, J 1.5), 8.29 (1H, br. d, J 7.9), 7.98 (1H, tented dd, J 8.5 and 0.5), 7.94 (1H, tented dd, J 8.5 and 1.5), 4.37 (1H, br. s), 3.84-3.71 (1H, m), 3.51 (2H, t, J6.3), 3.12 (1H, tt, J 11.4 and 3.8), 3.00-2.92 (2H, m), 2.42 (2H, t, J 6.3), 2.20-2.04 (4H, m), 1.89-1.67 (6H, m), 1.65-1.57 (1H, m), 1.39-1.22 (4H, m), 1.20-1.07 (1H, m).

### Example 174: N-(4,4-difluorocyclohexyl)-2-(1-(2-hydroxyethyl)piperidin-4-yl)benzo[d]thiazole-6-carboxamide

Prepared according to Scheme 12 starting from N-(4,4-difluorocyclohexyl)-2-(piperidin-4-yl)benzo[d]thiazole-6-carboxamide (Example 136).

¹H NMR (free base form): δ_{H} (400 MHz, DMSO-d₆) 8.55 (1H, dd, J 1.6 and 0.5), 8.41 (1H, br. d, J7.8), 7.99 (1H, tented dd, J 8.5 and 0.5), 7.95 (1H, tented dd, J 8.5 and 1.6), 4.37 (1H, br. s), 4.07-3.96 (1H, m), 3.51 (2H, t, J 6.3), 3.12 (1H, tt, J 11.4 and 3.8), 3.00-2.92 (2H, m), 2.41 (2H, t, J 6.3), 2.19-1.74 (12H, m), 1.72-1.59 (2H, m).

### Example 175: (S)-N-(chroman-4-yl)-2-(1-(2-(2-methoxyethoxy)ethyl)piperidin-4-yl)benzo[d]thiazole-6-carboxamide

Example 175 may be prepared according to the route shown in Scheme 13.

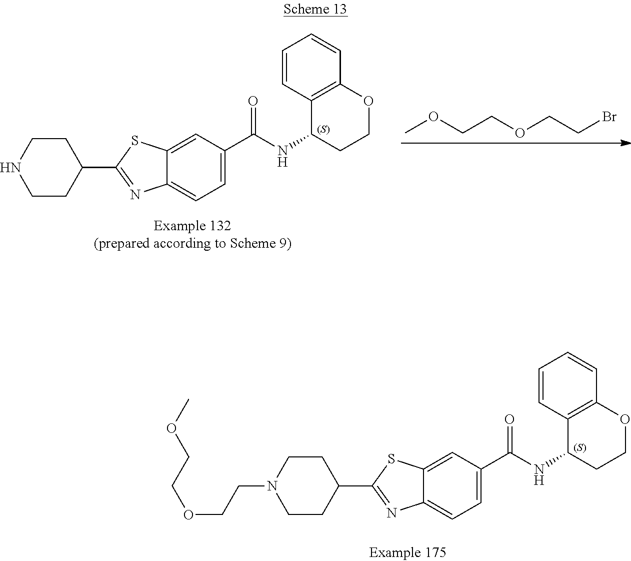

### Example 132 Example 175

To a stirred solution of (S)-N-(chroman-4-yl)-2-(piperidin-4-yl)benzo[d]thiazole-6-carboxamide (Example 132; 1.0 equiv.) in acetonitrile (1 volume; 0.05 M in substrate) was added K₂CO₃ (2.0 equiv.) and 1-bromo-2-(2-methoxyethoxy)ethane (1.0 equiv.). The reaction mixture was heated at 50° C. with stirring for 16 h and then cooled, diluted with water (2.5 volumes) and extracted with 5% v/v MeOH/DCM (5 volumes×3). The combined organic extract was washed with brine (2.5 volumes), dried (Na₂SO₄) and evaporated to dryness under reduced pressure. The resulting residue was processed by preparative HPLC to afford the (S)-N-(chroman-4-yl)-2-(1-(2-(2-methoxyethoxy)ethyl)piperidin-4-yl)benzo[d]thiazole-6-carboxamide as an off-white solid (23% yield).

¹H NMR (free base form): δ_{H} (400 MHz, DMSO-d₆) 8.97 (1H, br. d, J8.1), 8.62 (1H, dd, J 1.6 and 0.5), 8.03 (1H, tented dd, J 8.6 and 1.6), 7.99 (1H, tented dd, J 8.6 and 0.5), 7.20 (1H, app. d, J7.6), 7.16 (1H, app. td, J7.7 and 1.5), 6.88 (1H, td, J7.5 and 1.1), 6.81 (1H, dd, J 8.2 and 1.1), 5.33-5.28 (1H, m), 4.34-4.22 (2H, m), 3.54-3.48 (4H, m), 3.46-3.41 (2H, m), 3.24 (3H, s), 3.12 (1H, tt, J 11.4 and 3.8), 3.00-2.92 (2H, m), 2.53-2.50 (2H, m), 2.20-2.01 (6H, m), 1.86-1.72 (2H, m).

### Examples 176 and 177

Examples 176 and 177 may be prepared according to the route shown in Scheme 14.

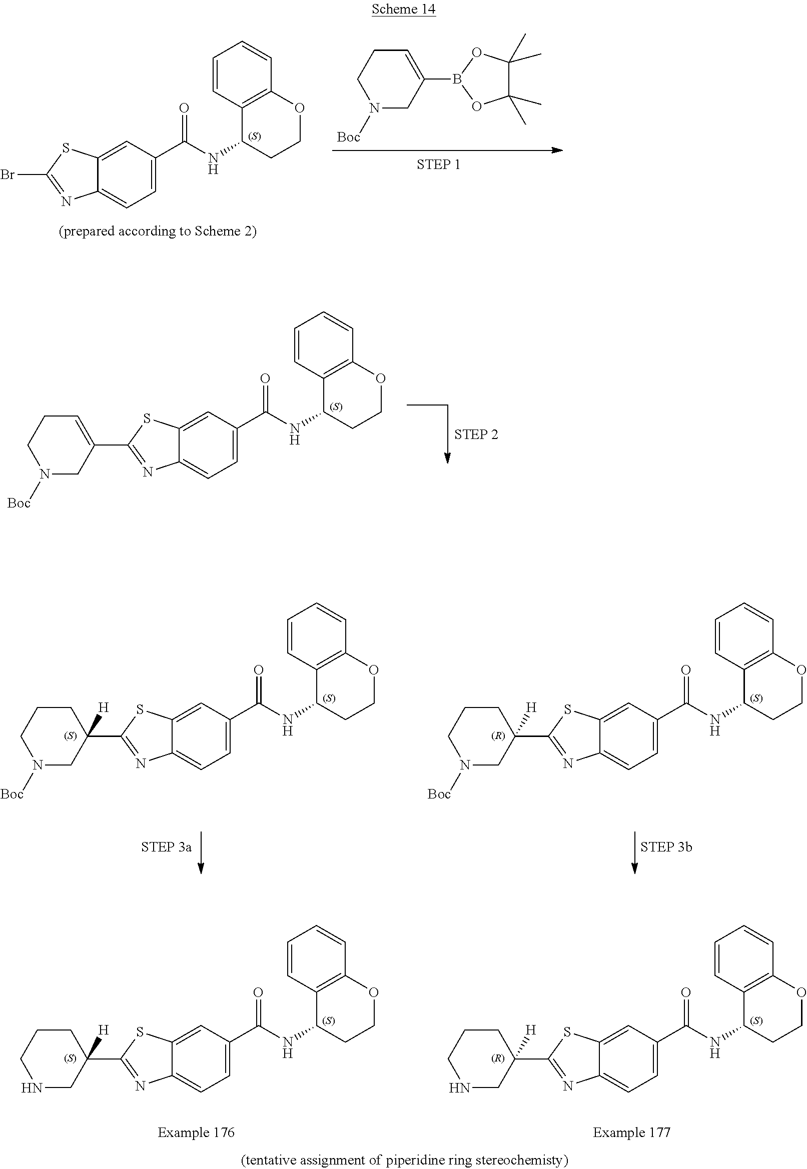

### Step 1 (Scheme 14): Synthesis of tert-butyl 3-(6-((S)-chroman-4-ylcarbamoyl)benzo[d]thiazol-2-yl)-5,6-dihydropyridine-1 (2H)-carboxylate

To a stirred, argon-purged mixture of (S)-2-bromo-N-(chroman-4-yl)benzo[d]thiazole-6-carboxamide (1 equiv.; prepared as described in Scheme 2) in 9:1 v/v 1,4-dioxane/H₂O (0.077 M in substrate) was added tert-butyl 5-(4,4,5,5-tetramethyl-1,3,2-dioxaborolan-2-yl)-3,6-dihydropyridine-1 (2H)-carboxylate (1.2 equiv.), K₂CO₃ (2.0 equiv.) and Pd(PPh₃)₄ (10 mol %).

The mixture was heated to reflux for 16 h under argon and then cooled and filtered through Celite®, washing with 5% v/v MeOH/DCM. The filtrate was concentrated in vacuo to obtain a residue that was dissolved in 5% v/v MeOH/DCM and washed with water followed by brine.

The organic layer was dried (Na₂SO₄) and evaporated to afford the crude product. The latter was processed by flash column chromatography (70% v/v EtOAc/hexanes) and fractions containing the target material combined and evaporated to afford tert-butyl 5-(6-((S)-chroman-4-ylcarbamoyl)benzo[d]thiazol-2-yl)-3,6-dihydropyridine-1 (2H)-carboxylate as an off-white solid (80% yield).

### Step 2 (Scheme 14): Synthesis of tert-butyl 3-(6-((S)-chroman-4-ylcarbamoyl)benzo[d]thiazol-2-yl)piperidine-1-carboxylate diastereoisomers

A solution of tert-butyl 5-(6-((S)-chroman-4-ylcarbamoyl)benzo[d]thiazol-2-yl)-3,6-dihydropyridine-1 (2H)-carboxylate in ethanol (0.026 M in substrate) was hydrogenated over PtO₂ at ambient temperature and pressure. After 16 h the reaction mixture was purged with argon and filtered through Celite®, washing with MeOH, and the filtrate concentrated under reduced pressure. The resulting residue was initially processed by flash column chromatography (60% v/v EtOAc/hexanes) and fractions containing the product isomers combined and evaporated. The isomer mixture thus obtained was then separated by SFC into two components: ‘Diastereoisomer-1’ (obtained in 18% yield) and ‘Diastereoisomer-2’ (obtained in 20% yield).

### Step 3a (Scheme 14) leading to Example 176: N-((S)-chroman-4-yl)-2-(piperidin-3-yl)benzo[d]thiazole-6-carboxamide ‘Diastereoisomer-1’

A solution of tert-butyl 3-(6-((S)-chroman-4-ylcarbamoyl)benzo[d]thiazol-2-yl)piperidine-1-carboxylate ‘Diastereoisomer-1’ (1.0 equiv.; obtained from Step 2) in DCM (0.12 M in substrate) was cooled to 0° C. with stirring prior to addition of 4 M HCl in 1,4-dioxane (2.7 equiv. HCl). The reaction mixture was brought to ambient temperature and stirred for 2-3 h. The mixture was then evaporated to obtain a residue that was processed by HPLC to afford the title compound as an off-white solid (84% yield). The product was tentatively assigned the (3S)-piperidin-3-yl stereochemistry of N-((S)-chroman-4-yl)-2-((S)-piperidin-3-yl)benzo[d]thiazole-6-carboxamide.

¹H NMR (free base form): δ_{H} (400 MHz, DMSO-d₆) 8.98 (1H, br. d, J8.1), 8.65 (1H, dd, J 1.6 and 0.5), 8.05 (1H, tented dd, J 8.6 and 1.6), 8.01 (1H, tented dd, J 8.6 and 0.5), 7.20 (1H, app. d, J7.6), 7.17 (1H, app. td, J7.7 and 1.5), 6.88 (1H, td, J7.5 and 1.1), 6.81 (1H, dd, J 8.2 and 1.1), 5.33-5.28 (1H, m), 4.35-4.22 (2H, m), 3.52-3.39 (3H, m), 3.14-2.99 (2H, m), 2.76 (1H, td, J 11.8 and 2.9), 2.26-2.02 (3H, m), 1.87-1.61 (3H, m).

### Step 3b (Scheme 14) leading to Example 177: N-((S)-chroman-4-yl)-2-(piperidin-3-yl)benzo[d]thiazole-6-carboxamide ‘Diastereoisomer-2’

The Boc group of tert-butyl 3-(6-((S)-chroman-4-ylcarbamoyl)benzo[d]thiazol-2-yl)piperidine-1-carboxylate ‘Diastereoisomer-2’, obtained from Step 2, was cleaved in a similar manner that of ‘Diastereoisomer-1’ described in Step 3a above. The product, obtained as an off-white solid (78% yield), was tentatively assigned the (3R)-piperidin-3-yl stereochemistry of N-((S)-chroman-4-yl)-2-((R)-piperidin-3-yl)benzo[d]thiazole-6-carboxamide.

¹H NMR (free base form): δ_{H} (400 MHz, DMSO-d₆) 8.98 (1H, br. d, J8.1), 8.65 (1H, app. d, J 1.6), 8.05 (1H, tented dd, J 8.6 and 1.6), 8.02 (1H, tented d, J 8.6), 7.20 (1H, app. d, J 7.6), 7.17 (1H, app. td, J 7.7 and 1.5), 6.88 (1H, td, J 7.5 and 1.1), 6.81 (1H, dd, J 8.2 and 1.1), 5.33-5.28 (1H, m), 4.35-4.22 (2H, m), 3.55-3.40 (3H, m), 3.17-3.02 (2H, m), 2.79 (1H, td, J 11.8 and 2.9), 2.27-2.02 (3H, m), 1.88-1.63 (3H, m).

Note that the identity of Examples 176 and 177 may potentially be reversed.

### Example 178: N-((S)-chroman-4-yl)-2-(pyrrolidin-3-yl)benzo[d]thiazole-6-carboxamide

Example 178 may be prepared according to the route shown in Scheme 15.

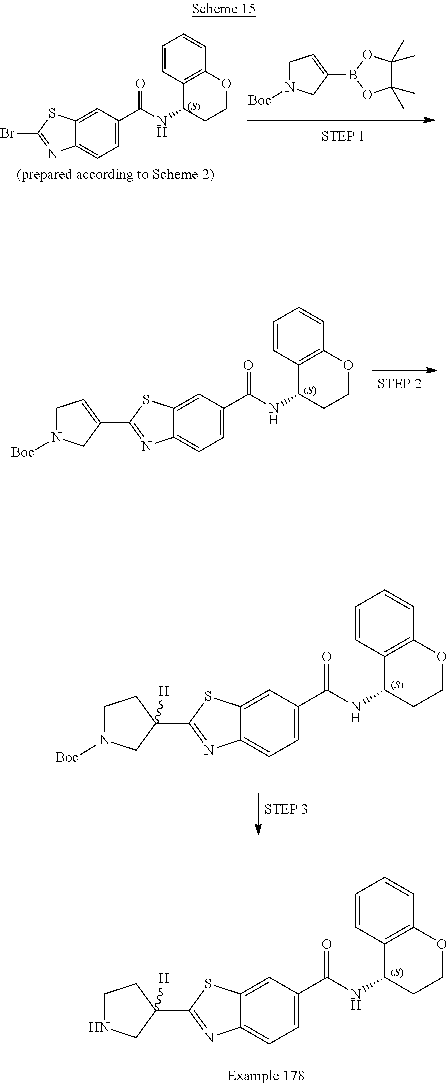

### Step 1 (Scheme 15): Synthesis of tert-butyl 3-(6-((S)-chroman-4-ylcarbamoyl)benzo[d]thiazol-2-yl)-2,5-dihydro-1H-pyrrole-1-carboxylate

To a stirred, argon-purged mixture of (S)-2-bromo-N-(chroman-4-yl)benzo[d]thiazole-6-carboxamide (1 equiv.; prepared as described in Scheme 2) in 4:1 v/v 1,4-dioxane/H₂O (0.065 M in substrate) was added tert-butyl 3-(4,4,5,5-tetramethyl-1,3,2-dioxaborolan-2-yl)-2,5-dihydro-1H-pyrrole-1-carboxylate (1.5 equiv.), K₂CO₃ (3.0 equiv.) and Pd(PPh₃)₄ (10 mol %).

The mixture was heated to reflux for 16 h under argon and then cooled and filtered through Celite®, washing with 5% v/v MeOH/DCM. The filtrate was concentrated in vacuo to obtain a residue that was dissolved in 5% v/v MeOH/DCM and washed with water followed by brine.

The organic layer was dried (Na₂SO₄) and evaporated to afford the crude product. The latter was processed by flash chromatography (40-50% v/v EtOAc/hexanes) and fractions containing the target material combined and evaporated to afford tert-butyl 3-(6-((S)-chroman-4-ylcarbamoyl)benzo[d]thiazol-2-yl)-2,5-dihydro-1H-pyrrole-1-carboxylate as an off-white solid (79% yield).

### Step 2 (Scheme 15): Synthesis of tert-butyl 3-(6-((S)-chroman-4-ylcarbamoyl)benzo[d]thiazol-2-yl)pyrrolidine-1-carboxylate, diastereomeric mixture

A solution of tert-butyl 3-(6-((S)-chroman-4-ylcarbamoyl)benzo[d]thiazol-2-yl)-2,5-dihydro-1H-pyrrole-1-carboxylate in ethanol (0.14 M in substrate) was hydrogenated over PtO₂ at ambient temperature and pressure. After 16 h the reaction mixture was purged with argon and filtered through Celite®, washing with MeOH, and the filtrate concentrated under reduced pressure.

The resulting residue was triturated with hexanes and dried in vacuo to afford tert-butyl 3-(6-((S)-chroman-4-ylcarbamoyl)benzo[d]thiazol-2-yl)pyrrolidine-1-carboxylate as an off-white solid (52% yield).

### Step 3 (Scheme 15) leading to Example 178: N-((S)-chroman-4-yl)-2-(pyrrolidin-3-yl)benzo[d]thiazole-6-carboxamide, diastereomeric mixture

A solution of tert-butyl 3-(6-((S)-chroman-4-ylcarbamoyl)benzo[d]thiazol-2-yl)pyrrolidine-1-carboxylate (1.0 equiv.; diastereomeric mixture obtained from Step 2) in DCM (0.084 M in substrate) was cooled to 0° C. with stirring prior to addition of 4 M HCl in 1,4-dioxane (2.4 equiv. HCl). The reaction mixture was brought to ambient temperature and stirred for 2-3 h. The mixture was then evaporated to obtain a residue that was triturated with pentane and dried in vacuo to afford N-((S)-chroman-4-yl)-2-(pyrrolidin-3-yl)benzo[d]thiazole-6-carboxamide (diastereomeric mixture) in its hydrochloride salt form as an off-white solid (91% yield).

¹H NMR (hydrochloride salt form): δ_{H} (400 MHz, DMSO-d₆) 9.24 (2H, br. s), 9.00 (1H, br. d, J 8.2), 8.67 (1H, app. d, J 1.6), 8.07 (1H, tented dd, J 8.6 and 1.6), 8.03 (1H, tented d, J 8.6), 7.20 (1H, app. d, J 7.6), 7.17 (1H, app. td, J 7.7 and 1.5), 6.88 (1H, td, J 7.5 and 1.1), 6.81 (1H, dd, J8.2 and 1.1), 5.33-5.28 (1H, m), 4.35-4.22 (2H, m), 4.14 (1H, pentet, J7.8), 3.77-3.67 (1H, m), 3.63-3.53 (1H, m), 3.44-3.24 (2H, m), 2.61-2.51 (1H, m), 2.30-2.21 (1H, m), 2.19-2.01 (2H, m).

### Example 179 [(S)-N-(2,3-dihydro-1H-inden-1-yl)-4-methyl-2-(piperidin-4-yl)benzo[d]thiazole-6-carboxamide] and Example 180 [(S)-N-(2,3-dihydro-1H-inden-1-yl)-2-(1-ethylpiperidin-4-yl)-4-methylbenzo[d]thiazole-6-carboxamide]

Example 179 and Example 180 may be prepared according to the illustrative route shown in Scheme 16.

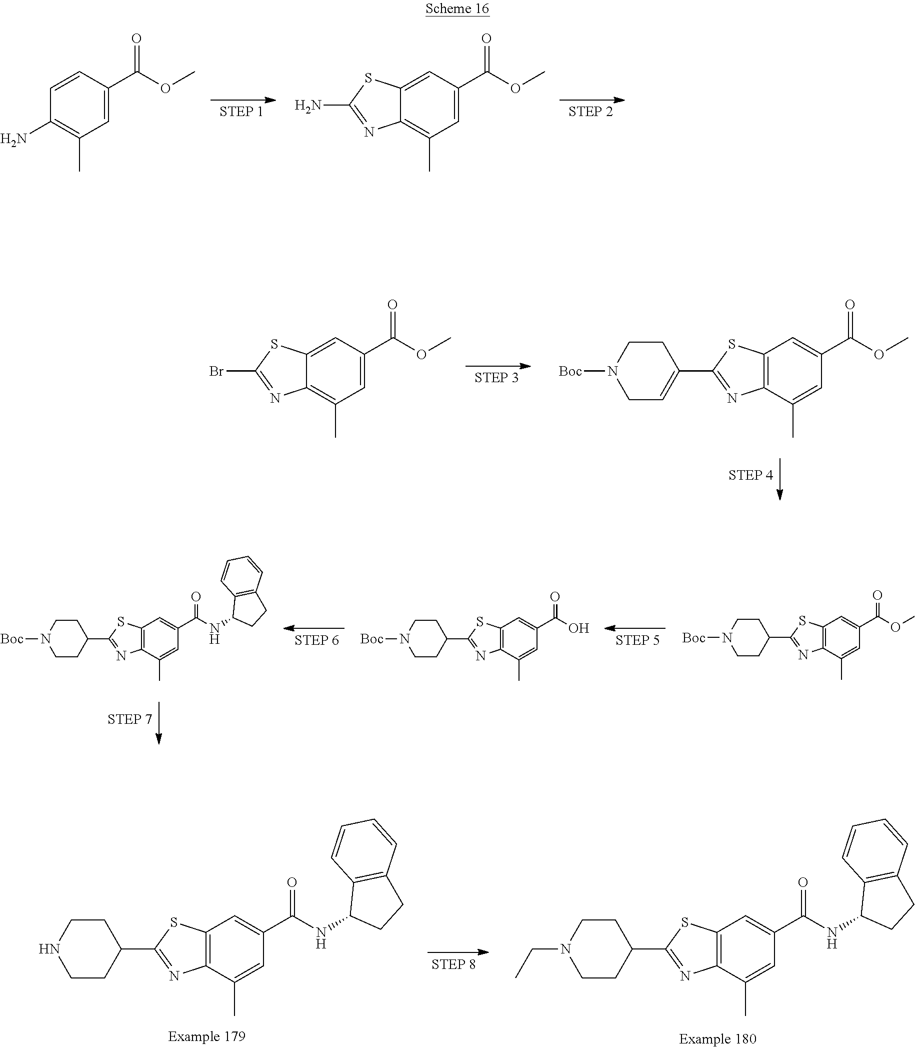

### Step 1 (Scheme 16): Preparation of 2-amino-4-methylbenzo[d]thiazole-6-carboxylate

A stirred mixture of methyl 4-amino-3-methylbenzoate (1.00 equiv.) and KSCN (4.00 equiv.) in AcOH (0.3 M solution of substrate) was cooled in an ice bath and a solution of Br₂ (0.67 M in AcOH; 1.11 equiv.) added dropwise. After 1 h (ca. half of the Br₂ solution addition) stirring failed and the mixture was thawed at ambient temperature, adding the remaining half of the Br₂ solution over a further period of 1 h. The resulting yellow slurry was then heated to 40° C. for 16 h with stirring. The mixture was then cooled, diluted with ice-water (2 volumes) and adjusted to pH 9 with ice-chilled NaOH solution (2 M aq). A yellow precipitate was deposited and collected by filtration, washing with water, and dried (80° C. oven initially and then in vacuo over P₂O₅) to afford crude methyl 2-amino-4-methylbenzo[d]thiazole-6-carboxylate as a yellow powder that was taken directly into the next Step 2.

¹H NMR: δ_{H} (300 MHz, DMSO-d₆) 8.12 (1H, app. s), 7.90 (2H, br. s), 7.66 (1H, app. s), 3.81 (3H, s), 2.44 (3H, s).

### Step 2 (Scheme 16): Preparation of 2-bromo-4-methylbenzo[d]thiazole-6-carboxylate

To an ice-cooled, stirred mixture of 2-amino-4-methylbenzo[d]thiazole-6-carboxylate (from the preceding step) and CuBr₂ (1.34 equiv. based on the quantity of 4-amino-3-methylbenzoate used in Step 1) in anhydrous MeCN (0.3 M solution of substrate) was added dropwise t-BuONO (2.02 equiv.). After complete addition the mixture was stirred for 48 h at 40° C. TLC (10% EtOAc/PE) indicated conversion of substrate (baseline) to target material (R_{f} 0.47). The reaction mixture was filtered through a short pad of silica, washing thoroughly with EtOAc, and the filtrate evaporated to afford a brown solid that was processed chromatographically over Merck Kieselgel 60H (DCM followed by 0.5% v/v MeOH/DCM elution). Fractions containing the target material were combined and evaporated to afford methyl 2-bromo-4-methylbenzo[d]thiazole-6-carboxylate as a pale yellow solid (92% combined yield across Step 1 and Step 2).

¹H NMR: δ_{H} (300 MHz, CDCl₃) 8.35 (1H, dq, J 1.6 and 0.6), 7.95 (1H, dq, J 1.7 and 0.8), 3.95 (3H, s), 2.74 (3H, app. s).

### Step 3 (Scheme 16): Preparation of methyl 2-(1-(tert-butoxycarbonyl)-1,2,3,6-tetrahydropyridin-4-yl)-4-methylbenzo[d]thiazole-6-carboxylate

To an argon-purged, stirred solution of methyl 2-bromo-4-methylbenzo[d]thiazole-6-carboxylate (1.00 equiv.) in 1,4-dioxane (0.084 M solution of substrate) and H₂O (0.072 volumes) was added tert-butyl 4-(4,4,5,5-tetramethyl-1,3,2-dioxaborolan-2-yl)-3,6-dihydropyridine-1 (2H)-carboxylate (1.15 equiv.), K₂CO₃ (2.00 equiv.) and Pd(PPh₃)₄ (6 mol %). The yellow mixture was then heated at 90° C. After 18 h TLC analysis (10% v/v EtOAc/hexane) revealed the presence of target material (R_{f} 0.12), a non-uv active/permanganate-staining component (R_{f} 0.24; hydrolysed boronic ester, tert-butyl 4-oxopiperidine-1-carboxylate) and residual bromobenzothiazole starting material (R_{f} 0.35). Further addition of boronic ester (0.15 equiv.) and K₂CO₃ (0.17 equiv.) was made and the mixture heated at 90° C. for 5 h. TLC analysis at this point also revealed the reaction to be incomplete, with bromide (R_{f} 0.35) still detectable. Further addition of boronic ester (0.08 equiv.) and K₂CO₃ (0.17 equiv.) was made and the mixture heated at 95° C. for 18 h, resulting in complete consumption of bromobenzothiazole starting material (R_{f} 0.35) by TLC analysis and formation of a brown mixture. The mixture was concentrated in vacuo to give a black slurry that was diluted with water (0.4 volumes) and extracted with EtOAc (2×0.32 volumes). The combined organics were washed with brine (0.2 volumes), dried (MgSO₄) and evaporated to give a black oil. The latter was processed by flash chromatography (10-20% v/v EtOAc/light petroleum gradient elution). Fractions containing the target material were combined, evaporated and pumped (60° C. at 10 mbar) to afford methyl 2-(1-(tert-butoxycarbonyl)-1,2,3,6-tetrahydropyridin-4-yl)-4-methylbenzo[d]thiazole-6-carboxylate as a yellow glass (92% yield).

¹H NMR: δ_{H} (300 MHz, CDCl₃) 8.38 (1H, dq, J 1.7 and 0.6), 7.92 (1H dq, J 1.7 and 0.8), 6.77-6.69 (1H, m), 4.23-4.16 (2H, m), 3.94 (3H, s), 3.67 (2H, t, J5.7), 2.85-2.79 (2H, m), 2.74 (3H, app. s), 1.50 (9H, s).

### Step 4 (Scheme 16): Preparation of methyl 2-(1-(tert-butoxycarbonyl)piperidin-4-yl)-4-methylbenzo[d]thiazole-6-carboxylate

A stirred mixture of methyl 2-(1-(tert-butoxycarbonyl)-1,2,3,6-tetrahydropyridin-4-yl)-4-methylbenzo[d]thiazole-6-carboxylate in 4:1 v/v EtOH/EtOAc (0.03 M solution of substrate) and 10% Pd-C (8.7 mol % Pd based on substrate) was purged with argon and then hydrogenated at ambient temperature and pressure. After 24 h the reaction mixture was purged with argon and sampled for NMR analysis, which revealed substantial conversion to target material with ca. 17% residual starting material remaining. The catalyst was supplemented with a further portion of 10% Pd-C (3.1 mol % Pd based on substrate) and hydrogenation at ambient temperature and pressure continued for a further 24 h. The bulk mixture was then purged with argon and filtered through Celite®, washing thoroughly with EtOAc. The filtrate was evaporated and pumped to afford methyl 2-(1-(tert-butoxycarbonyl)piperidin-4-yl)-4-methylbenzo[d]thiazole-6-carboxylate as a viscous colourless oil (94% yield).

¹H NMR: δ_{H} (300 MHz, CDCl₃) 8.40 (1H, dq, J 1.7 and 0.6), 7.93 (1H dq, J 1.7 and 0.8), 4.32-4.14 (2H, m), 3.94 (3H, s), 3.30 (1H, tt, J 11.5 and 3.8), 3.01-2.85 (2H, m), 2.74 (3H, app. s), 2.22-2.11 (2H, m), 1.91-1.77 (2H, m), 1.48 (9H, s).

### Step 5 (Scheme 16): Preparation of 2-(1-(tert-butoxycarbonyl)piperidin-4-yl)-4-methylbenzo[d]thiazole-6-carboxylic acid

To a stirred solution of methyl 2-(1-(tert-butoxycarbonyl)piperidin-4-yl)-4-methylbenzo[d]thiazole-6-carboxylate (1.00 equiv.) in 2:2:1 v/v/v THF/MeOH/water (0.1 M solution of substrate) was added LiOH (4.21 equiv.). The mixture was stirred at ambient temperature for 18 h. TLC analysis (EtOAc/2 M HCl aq sample partition; run in 30% v/v EtOAc/light petroleum) indicated complete conversion of starting material (R_{f} 0.32) to target material (baseline). The mixture was then concentrated in vacuo to remove the organic solvents and the residue adjusted to pH ca. 2 with 2 M HCl aq and extracted with EtOAc (2×0.5 volumes). The combined organic extract was washed with brine (0.3 volumes), dried (MgSO₄) and evaporated to afford 2-(1-(tert-butoxycarbonyl)piperidin-4-yl)-4-methylbenzo[d]thiazole-6-carboxylic acid as a cream coloured solid (91% yield).

¹H NMR: δ_{H} (300 MHz, DMSO-d₆) 8.50 (1H, dq, J 1.7 and 0.6), 7.86 (1H dq, J 1.7 and 0.8), 4.10-3.98 (2H, m), 3.02-2.86 (2H, m), 2.67 (3H, app. s), 2.17-2.06 (2H, m), 1.65 (2H, qd, J 12.3 and 4.0), 1.42 (9H, s).

### Step 6 (Scheme 16): Preparation of tert-butyl (S)-4-(6-((2,3-dihydro-1H-inden-1-yl)carbamoyl)-4-methylbenzo[d]thiazol-2-yl)piperidine-1-carboxylate

A mixture of methyl 2-(1-(tert-butoxycarbonyl)piperidin-4-yl)-4-methylbenzo[d]thiazole-6-carboxylic acid (1.00 equiv.), 1-[bis(dimethylamino)methylene]-1H-1,2,3-triazolo[4,5-b]pyridinium 3-oxide hexafluorophosphate (HATU; 1.29 equiv.) and DIPEA (2.05 equiv.) in DMF (0.09 M solution of substrate) was stirred at ambient temperature for 10 min. (S)-1-aminoindane (1.61 equiv.) was then added, stirring overnight at ambient temperature. TLC analysis (5% v/v MeOH/DCM) confirmed conversion of the substrate (R_{f} 0.14) to a faster running product component (R_{f} 0.56). The reaction mixture was evaporated to dryness (60° C. at 7 mbar) and the resulting residue made up into solution with EtOAc. The solution was washed successively with brine, 0.05 M citric acid solution and brine. It was then dried (MgSO₄) and evaporated to afford a brown wax that was processed by column chromatography (silica gel; 100% DCM to 1% MeOH/DCM gradient elution). Fractions containing clean target material were combined and evaporated to afford tert-butyl (S)-4-(6-((2,3-dihydro-1H-inden-1-yl)carbamoyl)-4-methylbenzo[d]thiazol-2-yl)piperidine-1-carboxylate as a buff-coloured solid (59% yield).

¹H NMR: δ_{H} (300 MHz, CDCl₃) 8.19 (1H, dq, J 1.7 and 0.5), 7.63 (1H, dq, J 1.7 and 0.8), 7.41-7.36 (1H, m), 7.32-7.21 (3H, m), 6.34 (1H, br. d, J 8.0), 5.73 (1H, q, J 7.6), 4.33-4.13 (2H, m), 3.30 (1H, tt, J 11.5 and 3.8), 3.11-2.87 (4H, m), 2.74 (3H, app. s), 2.79-2.68 (1H, m), 2.22-2.11 (2H, m), 2.02-1.77 (3H, m), 1.48 (9H, s).

### Step 7 (Scheme 16) leading to Example 179: (S)-N-(2,3-dihydro-1H-inden-1-yl)-4-methyl-2-(piperidin-4-yl)benzo[d]thiazole-6-carboxamide

tert-Butyl (S)-4-(6-((2,3-dihydro-1H-inden-1-yl)carbamoyl)-4-methylbenzo[d]thiazol-2-yl)piperidine-1-carboxylate was treated with 4 N HCl in dioxane (70 equiv. HCl), sonicating for 20 min at ambient temperature. The reaction mixture was evaporated to dryness and the residue partitioned between Na₂CO₃ solution (1 volume) and EtOAc (5 volumes). The organic extract was dried (MgSO₄) and evaporated to afford the (S)-N-(2,3-dihydro-1H-inden-1-yl)-4-methyl-2-(piperidin-4-yl)benzo[d]thiazole-6-carboxamide as a buff-coloured powder (94% yield).

¹H NMR (as free base): δ_{H} (300 MHz, CDCl₃) 8.18 (1H, dq, J 1.7 and 0.5), 7.62 (1H, dq, J 1.7 and 0.8), 7.41-7.35 (1H, m), 7.31-7.26 (2H, m), 7.26-7.21 (1H, m), 6.37 (1H, br. d, J7.8), 5.73 (1H, q, J7.6), 3.27 (1H, tt, J 11.5 and 3.8), 3.26-3.18 (2H, m), 3.06 (1H, tented ddd, J 16.0, 8.8 and 4.2), 2.94 (1H, tented dt, J 16.0 and 8.0), 2.80 (2H, td, J 12.6 and 3.2), 2.74 (3H, app. s), 2.78-2.67 (1H, m), 2.22-2.12 (2H, m), 2.04-1.87 (1H, m), 1.75-1.89 (2H, dtd, J 13.1, 11.9 and 4.1), 1.60 (1H, br. s).

### Step 8 (Scheme 16) leading to Example 180: (S)-N-(2,3-dihydro-1H-inden-1-yl)-2-(1-ethylpiperidin-4-yl)-4-methylbenzo[d]thiazole-6-carboxamide

A mixture of (S)-N-(2,3-dihydro-1H-inden-1-yl)-4-methyl-2-(piperidin-4-yl)benzo[d]thiazole-6-carboxamide (1.00 equiv.) and freshly distilled acetaldehyde (3.00 equiv.) in 1,2-dichloroethane (1 volume; 0.02 M solution in substrate) was cooled to 0° C., stirring for 5 min. Sodium triacetoxyborohydride (3.00 equiv.) was then added, stirring for 30 min at 0° C. followed by 1.5 h at ambient temperature. The mixture was then partitioned between 2 M aqueous Na₂CO₃ solution (1 volume) and DCM (1 volume). The organic phase was separated and the aqueous layer further extracted with DCM (1 volume). The combined organic phases were washed with brine (1 volume), dried (Na₂SO₄) and evaporated to afford a yellow solid that was processed by column chromatography (silica gel; 2-5% MeOH/DCM gradient elution). Fractions containing the clean product were combined and evaporated to afford (S)-N-(2,3-dihydro-1H-inden-1-yl)-2-(1-ethylpiperidin-4-yl)-4-methylbenzo[d]thiazole-6-carboxamide as a pale yellow powder (65% yield).

¹H NMR (as free base): δ_{H} (300 MHz, CDCl₃) 8.17 (1H, dq, J 1.7 and 0.5), 7.62 (1H, dq, J 1.7 and 0.8), 7.40-7.35 (1H, m), 7.30-7.26 (2H, m), 7.26-7.20 (1H, m), 6.38 (1H, br. d, J 8.3), 5.72 (1H, q, J7.7), 3.17 (1H, tt, J 11.5 and 3.8), 3.12-3.00 (3H, m), 2.99-2.88 (1H, m), 2.73 (3H, app. s), 2.78-2.66 (1H, m), 2.48 (2H, q, J 7.2), 2.25-2.06 (4H, m), 2.05-1.89 (3H, m), 1.13 (3H, t, J 7.2).

### Example 181: N-cyclopentyl-4-methyl-2-(piperidin-4-yl)benzo[d]thiazole-6-carboxamide

Example 181 may be prepared by analogy to Example 179 of Scheme 16 but using cyclopentylamine in Step 6 in place of (S)-1-aminoindane.

¹H NMR (as free base): δ_{H} (300 MHz, CDCl₃) 8.12 (1H, app. d, J 1.7), 7.57 (1H, dq, J 1.7 and 0.8), 6.11 (1H, br. d, J7.1), 4.42 (1H, sextet, J7.0), 3.26 (1H, tt, J 11.5 and 3.8), 3.22 (2H, dt, J 12.0 and 3.6), 2.79 (2H, td, J 12.2 and 2.5), 2.73 (3H, app. s), 2.19-2.05 (4H, m), 1.88-1.45 (9H, m (inc. piperidine NH)).

### Example 182: N-cyclopentyl-2-(1-ethylpiperidin-4-yl)-4-methylbenzo[d]thiazole-6-carboxamide

Example 182 may be prepared by analogy to Example 180 of Scheme 16 but using cyclopentylamine in Step 6 in place of (S)-1-aminoindane.

¹H NMR (as free base): δ_{H} (300 MHz, CDCl₃) 8.10 (1H, app. d, J 1.7), 7.57 (1H, dq, J 1.7 and 0.8), 6.13 (1H, br. d, J 7.4), 4.41 (1H, sextet, J 7.0), 3.15 (1H, tt, J 11.3 and 3.9), 3.12-3.02 (2H, m), 2.72 (3H, app. s), 2.45 (2H, q, J 7.2), 2.22-2.04 (6H, m), 2.03-1.89 (2H, m), 1.80-1.59 (4H, m), 1.58-1.45 (2H, m), 1.12 (3H, t, J 7.2).

### Example 183: N-cyclopentyl-4-methyl-2-(1-methylpiperidin-4-yl)benzo[d]thiazole-6-carboxamide

Example 183 may be prepared by reductive amination of Example 181 with formaldehyde as follows. To a stirred solution of N-cyclopentyl-4-methyl-2-(piperidin-4-yl)benzo[d]thiazole-6-carboxamide (Example 181; 1.0 equiv.) in MeOH (1 volume; 0.043 M in substrate) was added formaldehyde (37% w/w in H₂O; 1.1 equiv.) and acetic acid (4.0 equiv.). The mixture was stirred at ambient temperature for 2 h and then cooled to 0° C. prior to addition of sodium cyanoborohydride (2.0 equiv.), stirring for a further period of 2 h. TLC analysis (10% v/v MeOH/CHCl₃) revealed complete and clean conversion of substrate (R_{f} 0.09) to a faster running product component (R_{f} 0.17). The reaction mixture was quenched with ice-cold water (1 volume), concentrated under reduced pressure and extracted with CHCl₃ (3×3 volumes). The combined CHCl₃ extract was washed with saturated NaHCO₃ solution (1 volume) followed by brine (1 volume), dried (Na₂SO₄) and evaporated to dryness under reduced pressure to afford N-cyclopentyl-4-methyl-2-(1-methylpiperidin-4-yl)benzo[d]thiazole-6-carboxamide as a white powder (64% yield).

¹H NMR (as free base): δ_{H} (300 MHz, CDCl₃) 8.12 (1H, app. d, J 1.7), 7.58 (1H, dq, J 1.7 and 0.8), 6.06 (1H, br. d, J7.1), 4.23 (1H, sextet, J7.0), 3.13 (1H, tt, J 11.3 and 3.9), 3.04-2.94 (2H, m), 2.74 (3H, app. s), 2.34 (3H, s), 2.23-1.89 (8H, m), 1.82-1.60 (4H, m), 1.59-1.45 (2H, m).

### Example 184: 4-methyl-2-(piperidin-4-yl)-N-(tetrahydro-2H-pyran-4-yl)benzo[d]thiazole-6-carboxamide

Example 184 may be prepared by analogy to Example 179 of Scheme 16 but using tetrahydro-2H-pyran-4-amine in Step 6 in place of (S)-1-aminoindane.

¹H NMR (as free base): δ_{H} (300 MHz, CDCl₃) 8.14 (1H, dq, J 1.7 and 0.5), 7.59 (1H, dq, J 1.7 and 0.8), 6.05 (1H, br. d, J 7.8), 4.23 (1H, tdt, J 11.3, 7.6 and 3.9), 4.06-3.96 (2H, m), 3.55 (2H, td, J 11.7 and 2.1), 3.27 (1H, tt, J 11.7 and 3.8), 3.26-3.19 (2H, m), 2.80 (2H, td, J 12.1 and 2.4), 2.75 (3H, app. s), 2.22-2.12 (2H, m), 2.08-1.99 (2H, m), 1.89-1.76 (2H, m), 1.71 (1H, br. s), 1.66-1.52 (2H, m).

### Example 185: 2-(1-ethylpiperidin-4-yl)-4-methyl-N-(tetrahydro-2H-pyran-4-yl)benzo[d]thiazole-6-carboxamide

Example 185 may be prepared by analogy to Example 180 of Scheme 16 but using tetrahydro-2H-pyran-4-amine in Step 6 in place of (S)-1-aminoindane

¹H NMR (as free base): δ_{H} (300 MHz, CDCl₃) 8.13 (1H, dq, J 1.7 and 0.5), 7.59 (1H, dq, J 1.7 and 0.8), 6.07 (1H, br. d, J 7.9), 4.22 (1H, tdt, J 11.3, 7.6 and 3.8), 4.05-3.96 (2H, m), 3.54 (2H, td, J 11.7 and 2.1), 3.17 (1H, tt, J 11.7 and 3.8), 3.14-3.05 (2H, m), 2.74 (3H, app. s), 2.48 (2H, q, J7.2), 2.50-1.91 (8H, m), 1.66-1.52 (2H, m), 1.13 (3H, t, J7.2).

### Example 186: (S)-N-(chroman-4-yl)-4-methyl-2-(piperidin-4-yl)benzo[d]thiazole-6-carboxamide

Example 186 may be prepared by analogy to Example 179 of Scheme 16 but using (S)-chroman-4-amine in Step 6 in place of (S)-1-aminoindane.

¹H NMR (as free base): δ_{H} (300 MHz, CDCl₃) 8.17 (1H, app. d, J 1.7), 7.61 (1H, dq, J 1.7 and 0.8), 7.29 (1H, dd, J 7.9 and 1.5), 7.25-7.19 (1H, m), 6.94 (1H, td, J 7.5 and 1.2), 6.88 (1H, dd, J 8.2 and 1.2), 6.42 (1H, br. d, J7.3), 5.36 (1H, app. q, J6.2), 4.33 (1H, tented ddd, J 11.5, 6.0 and 3.5), 4.23 (1H, tented ddd, J 11.5, 8.8 and 2.8), 3.27 (1H, tt, J 11.7 and 3.8), 3.27-3.18 (2H, m), 2.79 (2H, td, J 12.1 and 2.3), 2.74 (3H, app. s), 2.34 (1H, tented dddd, J 14.1, 8.9, 5.2 and 3.6), 2.26-2.11 (3H, m), 1.89-1.75 (2H, m), 1.67 (1H, br. s).

### Example 187: (S)-N-(chroman-4-yl)-2-(1-ethylpiperidin-4-yl)-4-methylbenzo[d]thiazole-6-carboxamide

Example 187 may be prepared by analogy to Example 180 of Scheme 16 but using (S)-chroman-4-amine in Step 6 in place of (S)-1-aminoindane.

¹H NMR (as free base): δ_{H} (300 MHz, CDCl₃) 8.16 (1H, dq, J 1.7 and 0.5), 7.61 (1H, dq, J 1.7 and 0.8), 7.29 (1H, dd, J7.9 and 1.5), 7.25-7.19 (1H, m), 6.94 (1H, td, J7.5 and 1.2), 6.88 (1H, dd, J8.2 and 1.2), 6.40 (1H, br. d, J7.3), 5.36 (1H, app. q, J6.3), 4.33 (1H, tented ddd, J 11.5, 6.1 and 3.6), 4.22 (1H, tented ddd, J 11.5, 8.8 and 2.9), 3.17 (1H, tt, J 11.4 and 3.8), 3.14-3.03 (2H, m), 2.74 (3H, app. s), 2.48 (2H, q, J 7.2), 2.39-2.29 (1H, m), 2.26-1.91 (7H, m), 1.13 (3H, t, J7.2).

### Example 188: N-cyclopentyl-5-methyl-2-(piperidin-4-yl)benzo[d]thiazole-6-carboxamide

Example 188 may be prepared by analogy to Example 179 of Scheme 16 but commencing with methyl 4-amino-2-methylbenzoate in Step 1 instead of methyl 4-amino-3-methylbenzoate and using cyclopentylamine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.82 (1H, s), 7.78 (1H, s), 5.75 (1H, br. d, J7.2), 4.42 (1H, app. sextet, J7.2), 3.27-3.17 (3H, m), 2.80 (2H, td, J 12.2 and 2.4), 2.55 (3H, s), 2.19-2.04 (4H, m), 1.89-1.59 (7H, m), 1.57-1.42 (2H, m).

### Example 189: N-cyclopentyl-2-(1-ethylpiperidin-4-yl)-5-methylbenzo[d]thiazole-6-carboxamide

Example 189 may be prepared by analogy to Example 180 of Scheme 16 but commencing with methyl 4-amino-2-methylbenzoate in Step 1 instead of methyl 4-amino-3-methylbenzoate and using cyclopentylamine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.80 (1H, s), 7.75 (1H, app. s), 5.79 (1H, br. d, J7.4), 4.40 (1H, app. sextet, J7.3), 3.15-3.02 (3H, m), 2.53 (3H, app. s), 2.45 (2H, q, J7.2), 2.22-2.02 (6H, m), 1.94 (2H, qd, J 11.8 and 3.0), 1.77-1.57 (4H, m), 1.57-1.41 (2H, m), 1.11 (3H, t, J7.2).

### Example 190: (S)-N-(chroman-4-yl)-5-methyl-2-(piperidin-4-yl)benzo[d]thiazole-6-carboxamide

Example 190 may be prepared by analogy to Example 179 of Scheme 16 but commencing with methyl 4-amino-2-methylbenzoate in Step 1 instead of methyl 4-amino-3-methylbenzoate and using (S)-chroman-4-amine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.80 (1H, app. s), 7.74 (1H, app. s), 7.28 (1H, dd, J 7.9 and 1.5), 7.20-7.14 (1H, m), 6.91 (1H, td, J 7.5 and 1.2), 6.82 (1H, dd, J 8.3 and 1.1), 6.50 (1H, br. d, J7.7), 5.32 (1H, dt, J7.6 and 5.3), 4.30 (1H, tented ddd, J 11.5, 6.1 and 3.4), 4.19 (1H, tented ddd, J 11.5, 8.9 and 2.7), 3.19-3.08 (3H, m), 2.71 (2H, td, J 12.1 and 2.3), 2.55 (3H, s), 2.32 (1H, tented dddd, J 14.1, 8.9, 5.2 and 3.6), 2.21-2.04 (3H, m), 1.83 (1H, br. s), 1.79-1.64 (2H, m).

### Example 191: (S)-N-(chroman-4-yl)-2-(1-ethylpiperidin-4-yl)-5-methylbenzo[d]thiazole-6-carboxamide

Example 191 may be prepared by analogy to Example 180 of Scheme 16 but commencing with methyl 4-amino-2-methylbenzoate in Step 1 instead of methyl 4-amino-3-methylbenzoate and using (S)-chroman-4-amine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.85 (1H, app. s), 7.78 (1H, app. s), 7.30 (1H, dd, J 7.9 and 1.5), 7.22-7.16 (1H, m), 6.93 (1H, td, J 7.5 and 1.2), 6.84 (1H, dd, J 8.3 and 1.1), 6.15 (1H, br. d, J7.5), 5.35 (1H, app. q, J6.3), 4.33 (1H, tented ddd, J11.5, 6.1 and 3.4), 4.20 (1H, tented ddd, J 11.5, 9.0 and 2.7), 3.21-3.02 (3H, m), 2.59 (3H, s), 2.52 (2H, q, J 7.2), 2.41-2.11 (6H, m), 2.11-1.90 (2H, m), 1.16 (3H, t, J 7.2).

### Example 192: N-cyclopentyl-7-methyl-2-(piperidin-4-yl)benzo[d]thiazole-6-carboxamide

Example 192 may be prepared by analogy to Example 179 of Scheme 16 but commencing with methyl 4-amino-2-methylbenzoate in Step 1 instead of methyl 4-amino-3-methylbenzoate and using cyclopentylamine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.83 (1H, dq, J 8.3 and 0.5), 7.48 (1H, d, J 8.3), 5.81 (1H, br. d, J 7.3), 4.45 (1H, app. sextet, J 7.2), 3.31-3.20 (3H, m), 2.83 (2H, td, J 12.2 and 2.4), 2.68 (3H, app. s), 2.23-2.08 (4H, m), 1.93-1.60 (7H, m), 1.47-1.59 (2H, m).

### Example 193: N-cyclopentyl-2-(1-ethylpiperidin-4-yl)-7-methylbenzo[d]thiazole-6-carboxamide

Example 193 may be prepared by analogy to Example 180 of Scheme 16 but commencing with methyl 4-amino-2-methylbenzoate in Step 1 instead of methyl 4-amino-3-methylbenzoate and using cyclopentylamine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.79 (1H, dq, J 8.3 and 0.5), 7.45 (1H, d, J 8.4), 5.75 (1H, br. d, J 7.3), 4.43 (1H, app. sextet, J 7.2), 3.18-3.04 (3H, m), 2.65 (3H, app. s), 2.49 (2H, q, J7.2), 2.28-1.94 (8H, m), 1.77-1.61 (4H, m), 1.57-1.42 (2H, m), 1.14 (3H, t, J 7.2).

### Example 194: (S)-N-(chroman-4-yl)-7-methyl-2-(piperidin-4-yl)benzo[d]thiazole-6-carboxamide

Example 194 may be prepared by analogy to Example 179 of Scheme 16 but commencing with methyl 4-amino-2-methylbenzoate in Step 1 instead of methyl 4-amino-3-methylbenzoate and using (S)-chroman-4-amine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.79 (1H, dq, J 1.7 and 0.5), 7.47 (1H, d, J8.4), 7.30 (1H, dd, J 7.7 and 1.3), 7.16-7.22 (1H, m), 6.93 (1H, td, J7.5 and 1.2), 6.84 (1H, dd, J 8.2 and 1.2), 6.28 (1H, br. d, J 7.6), 5.35 (1H, dt, J 7.6 and 5.3), 4.33 (1H, tented ddd, J 11.5, 6.2 and 3.5), 4.20 (1H, tented ddd, J 11.5, 8.9 and 2.7), 3.26-3.14 (3H, m), 2.76 (2H, td, J 12.1 and 2.4), 2.70 (3H, app. s), 2.36 (1H, tented dddd, J 14.1, 8.9, 5.2 and 3.6), 2.25-2.09 (3H, m), 1.86-1.71 (2H, m), 1.69 (1H, br. s).

### Example 195: (S)-N-(chroman-4-yl)-2-(1-ethylpiperidin-4-yl)-7-methylbenzo[d]thiazole-6-carboxamide

Example 195 may be prepared by analogy to Example 180 of Scheme 16 but commencing with methyl 4-amino-2-methylbenzoate in Step 1 instead of methyl 4-amino-3-methylbenzoate and using (S)-chroman-4-amine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.79 (1H, dq, J 8.3 and 0.5), 7.48 (1H, d, J 8.3), 7.30 (1H, app. dd, J 7.7 and 1.3), 7.23-7.17 (1H, m), 6.93 (1H, td, J7.5 and 1.2), 6.85 (1H, dd, J 8.2 and 1.2), 6.12 (1H, br. d, J7.8), 5.36 (1H, dt, J7.6 and 5.4), 4.33 (1H, tented ddd, J 11.5, 6.2 and 3.4), 4.20 (1H, tented ddd, J 11.5, 8.9 and 2.7), 3.17-3.04 (3H, m), 2.71 (3H, app. s), 2.48 (2H, q, J7.2), 2.36 (1H, tented dddd, J 14.1, 8.9, 5.2 and 3.6), 2.25-2.15 (3H, m), 2.15-2.07 (2H, m), 2.06-1.92 (2H, m), 1.13 (3H, t, J7.2).

### Example 196: (S)-N-(2,3-dihydro-1H-inden-1-yl)-2-(1-ethylpiperidin-4-vy)-7-methylbenzo[d]thiazole-6-carboxamide

Example 196 may be prepared by analogy to Example 180 of Scheme 16 but commencing with methyl 4-amino-2-methylbenzoate in Step 1 instead of methyl 4-amino-3-methylbenzoate. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.79 (1H, dq, J8.3 and 0.5), 7.51 (1H, d, J8.3), 7.42-7.36 (1H, m), 7.30-7.21 (3H, m), 6.04 (1H, br. d, J 8.4), 5.71 (1H, app. q, J7.7), 3.18-2.88 (5H, m), 2.79-2.67 (1H, m), 2.72 (3H, app. s), 2.48 (2H, q, J 7.2), 2.29-1.87 (7H, m), 1.14 (3H, t, J 7.2).

### Example 197: 2-(1-ethylpiperidin-4-yl)-7-methyl-N-(tetrahydro-2H-pyran-4-yl)benzo[d]thiazole-6-carboxamide

Example 197 may be prepared by analogy to Example 180 of Scheme 16 but commencing with methyl 4-amino-2-methylbenzoate in Step 1 instead of methyl 4-amino-3-methylbenzoate and using tetrahydro-2H-pyran-4-amine in Step 6 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.80 (1H, dq, J 8.3 and 0.5), 7.46 (1H, d, J 8.3), 5.74 (1H, br. d, J 7.9), 4.28-4.15 (1H, m), 4.04-3.94 (2H, m), 3.54 (2H, td, J 11.7 and 2.1), 3.18-3.04 (3H, m), 2.65 (3H, app. s), 2.49 (2H, q, J 7.2), 2.28-1.92 (8H, m), 1.63-1.49 (2H, m), 1.14 (3H, t, J7.2).

### Example 198: (S)-N-(2,3-dihydro-1H-inden-1-yl)-5-methoxy-2-(piperidin-4-yl)benzo[d]thiazole-6-carboxamide

Example 198 may be prepared by analogy to Example 180 of Scheme 16 but commencing with methyl 4-amino-2-methoxybenzoate in Step 1 instead of methyl 4-amino-3-methylbenzoate. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.77 (1H, s), 8.22 (1H, br. d, J 8.0), 7.54 (1H, s), 7.40-7.36 (1H, m), 7.31-7.21 (3H, m), 5.74 (1H, app. q, J 7.8), 3.95 (3H, s), 3.34-2.70 (9H, m), 2.27-2.18 (2H, m), 2.01-1.86 (3H, m).

### Example 199: N-cyclopentyl-5-methoxy-2-(piperidin-4-yl)benzo[d]thiazole-6-carboxamide

Example 199 may be prepared by analogy to Example 180 of Scheme 16 but commencing with methyl 4-amino-2-methoxybenzoate in Step 1 instead of methyl 4-amino-3-methylbenzoate and using cyclopentylamine in Step 6 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.70 (1H, s), 7.95 (1H, br. d, J7.2), 7.53 (1H, s), 4.43 (1H, pp. sextet, J 6.7), 4.01 (3H, s), 3.29-3.17 (3H, m), 2.82 (2H, td, J 12.1 and 2.4), 2.38 (1H, br. s), 2.24-2.14 (2H, m), 2.14-2.02 (2H, m), 1.94-1.80 (2H, m), 1.80-1.61 (4H, m), 1.60-1.47 (2H, m).

### Example 200: 5-methoxy-2-(piperidin-4-yl)-N-(tetrahydro-2H-pyran-4-yl)benzo[d]thiazole-6-carboxamide

Example 200 may be prepared by analogy to Example 180 of Scheme 16 but commencing with methyl 4-amino-2-methoxybenzoate in Step 1 instead of methyl 4-amino-3-methylbenzoate and using tetrahydro-2H-pyran-4-amine in Step 6 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.70 (1H, s), 7.95 (1H, br. d, J7.7), 7.55 (1H, s), 4.33-4.20 (1H, m), 4.04 (3H, s), 3.98 (2H, app. dt, J 11.8 and 3.7), 3.58 (2H, td, J 11.4 and 2.0), 3.28-3.17 (3H, m), 2.82 (2H, td, J 12.1 and 2.5), 2.24-2.14 (2H, m), 2.13-1.95 (3H, m), 1.86 (2H, tdd, J 12.4, 12.0 and 3.7), 1.60 (2H, dtd, J 13.1, 10.7 and 4.3).

### Example 201: (S)-N-(chroman-4-yl)-5-methoxy-2-(piperidin-4-yl)benzo[d]thiazole-6-carboxamide

Example 201 may be prepared by analogy to Example 180 of Scheme 16 but commencing with methyl 4-amino-2-methoxybenzoate in Step 1 instead of methyl 4-amino-3-methylbenzoate and using (S)-chroman-4-amine in Step 6 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.74 (1H, s), 8.19 (1H, br. d, J7.4), 7.53 (1H, s), 7.33 (1H, app. d, J7.8), 7.23-7.17 (1H, m), 6.92 (1H, td, J7.5 and 1.2), 6.87 (1H, dd, J 8.2 and 1.1), 5.42 (1H, app. q, J6.4), 4.33 (1H, tented ddd, J 11.0, 7.3 and 3.5), 4.24 (1H, tented ddd, J 11.3, 8.1 and 3.1), 3.93 (3H, s), 3.31-3.14 (3H, m), 2.84 (2H, td, J 12.1 and 2.3), 2.43-2.33 (1H, m), 2.26-2.11 (4H, m), 1.96-1.83 (2H, m).

### Example 202: (S)-N-(2,3-dihydro-1H-inden-1-yl)-4-methyl-2-(piperazin-1-yl)benzo[d]thiazole-6-carboxamide

Example 202 may be prepared according to the illustrative route shown in Scheme 17.

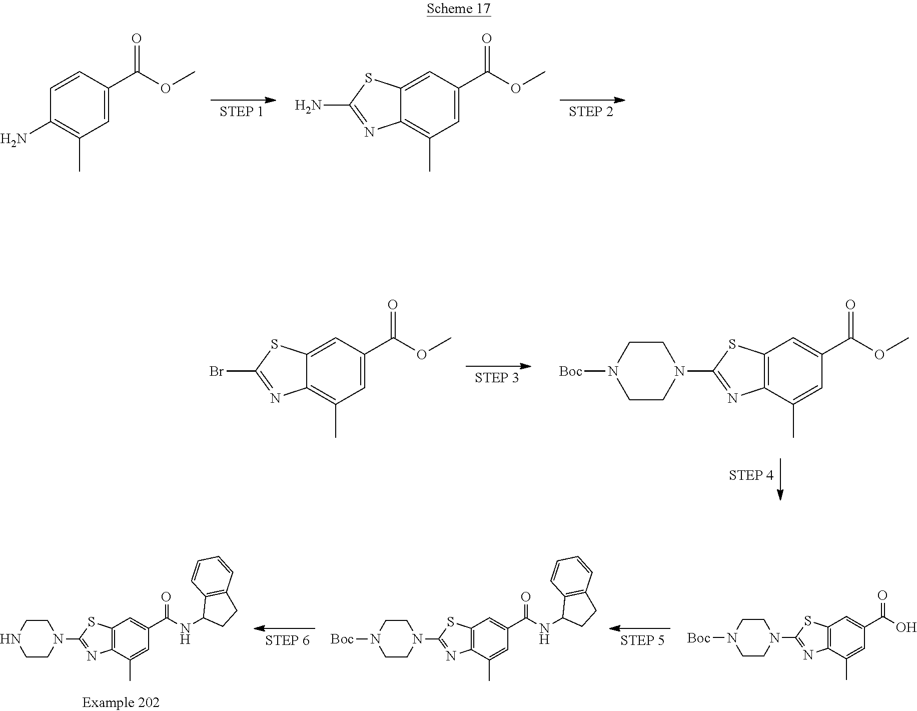

### Step 1 (Scheme 17): Preparation of 2-amino-4-methylbenzo[d]thiazole-6-carboxylate

As described for Examples 179 and 180 above (Scheme 16).

### Step 2 (Scheme 17): Preparation of 2-bromo-4-methylbenzo[d]thiazole-6-carboxylate

As described for Examples 179 and 180 above (Scheme 16).

### Step 3 (Scheme 17): Preparation of methyl 2-(4-(tert-butoxycarbonyl)piperazin-1-yl)-4-methylbenzo[d]thiazole-6-carboxylate

A stirred mixture of methyl 2-bromo-4-methylbenzo[d]thiazole-6-carboxylate (1.00 equiv. 10.9 mmol), powdered K₂CO₃ (2.00 equiv.) and tert-butyl piperazine-1-carboxylate (1.20 equiv.) in MeCN (0.09 M solution of substrate) was heated under Ar at 88° C. (sealed pressure vessel).

After 16 h, TLC analysis (25% v/v EtOAc/hexane) indicated complete conversion of substrate (R_{f} 0.60) to product (R_{f} 0.42). The mixture was cooled and filtered through Celite®, washing thoroughly with EtOAc. The filtrate was evaporated to dryness and the residue then reconstituted with EtOAc (1 volume) and washed successively with water (2×0.2 volumes), citric acid solution (0.01 M aq; 2×0.2 volumes) and saturated NaHCO₃ solution (1×0.2 volume). The organic phase was dried (MgSO₄) and evaporated to afford a yellow residue that was processed by flash chromatographically [10-20% v/v EtOAc/hexane gradient elution (column loading assisted with DCM)]. Fractions containing the target material were combined and evaporated to afford methyl 2-(4-(tert-butoxycarbonyl)piperazin-1-yl)-4-methylbenzo[d]thiazole-6-carboxylate as a pale yellow powder (91% yield).

¹H NMR: δ_{H} (300 MHz, CDCl₃) 8.16 (1H, dq, J 1.7 and 0.6), 7.82 (1H dq, J 1.7 and 0.8), 3.90 (3H, s), 3.65-3.69 (4H, m), 3.57-3.61 (4H, m), 2.56 (3H, app. s), 1.49 (9H, s).

### Step 4 (Scheme 17): Preparation of 2-(4-(tert-butoxycarbonyl)piperazin-1-yl)-4-methylbenzo[d]thiazole-6-carboxylic acid

To a stirred solution of methyl 2-(4-(tert-butoxycarbonyl)piperazin-1-yl)-4-methylbenzo[d]thiazole-6-carboxylate (1.00 equiv.) in 2:2:1 v/v/v THF/MeOH/water (0.07 M in substrate) was added LiOH (4.00 equiv.). The mixture was stirred at ambient temperature for 48 h. TLC analysis (25% v/v EtOAc/hexane) indicated the presence of some residual starting material (R_{f} 0.42) along with hydrolysis product (baseline). A further portion of LiOH (1.00 eq.) was added and the reaction mixture sonicated at 40° C. to drive the reaction to completion (confirmed by TLC analysis). The mixture was then concentrated in vacuo to the remove organic solvents, diluted with brine (1.5 volumes), acidified to pH ca. 2 and extracted with CHCl₃ (3×1.5 volumes) followed by 5% v/v MeOH/CHCl₃ (3×2 volumes). The combined organic extract was dried (MgSO₄), filtered through Celite® and evaporated to afford a solid residue. The latter was dried, initially in an oven at 80° C. and then in vacuo over P₂O₅, to afford crude 2-(4-(tert-butoxycarbonyl)piperazin-1-yl)-4-methylbenzo[d]thiazole-6-carboxylic acid as a white solid (99% yield).

¹H NMR: δ_{H} (300 MHz, DMSO-d₆) 8.18 (1H, app. s), 7.72 (1H, app. s), 3.66-3.44 (8H, m), 2.46 (3H, s), 1.43 (9H, s). (The spectrum exhibited signal broadening and the carboxylic acid was not detectable as a distinct resonance.)

### Step 5 (Scheme 17): Preparation of tert-butyl (S)-4-(6-((2,3-dihydro-1H-inden-1-yl)carbamoyl)-4-methylbenzo[d]thiazol-2-yl)piperazine-1-carboxylate

To a stirred solution of 2-(4-(tert-butoxycarbonyl)piperazin-1-yl)-4-methylbenzo[d]thiazole-6-carboxylic acid (1.00 equiv.), N-(3-dimethylaminopropyl)-N′-ethylcarbodiimide hydrochloride (EDCI·HCl; 1.20 equiv.) and N-hydroxybenzotriazole (HOBt; 1.20 equiv.) in DMF (0.08 M in substrate) was added Et₃N (2.50 equiv.) followed after 20 min by (S)-1-aminoindane (1.20 equiv.). After stirring for 48 h at ambient temperature, TLC analysis (10% v/v MeOH/DCM) indicated conversion of starting material (R_{f} 0.27) to product (R_{f} 0.46). The mixture was evaporated to dryness (60° C. at 7 mbar) to afford a residue that was taken up in EtOAc (1 volume) and washed successively with citric acid solution (0.01 M aq; 0.2 volumes), saturated NaHCO₃ solution (0.2 volumes) and brine (0.2 volumes). The organic phase was dried (MgSO₄) and evaporated to afford a brown solid that was processed chromatographically (silica gel; 0.25-0.50% v/v MeOH/DCM gradient elution). Fractions containing clean target material were combined and evaporated to afford tert-butyl (S)-4-(6-((2,3-dihydro-1H-inden-1-yl)carbamoyl)-4-methylbenzo[d]thiazol-2-yl)piperazine-1-carboxylate as a white powder (79% yield).

¹H NMR: δ_{H} (300 MHz, CDCl₃) 7.97 (1H, dq, J 1.7 and 0.5), 7.50 (1H, dq, J1.7 and 0.8), 7.39-7.35 (1H, m), 7.29-7.19 (3H, m), 6.35 (1H, br. d, J 8.3), 5.70 (1H, app. q, J 7.7), 3.68-3.61 (4H, m), 3.61-3.55 (4H, m), 3.04 (1H, tented ddd, J 15.9, 8.8 and 4.1), 2.92 (1H, tented dt, J 15.9 and 8.0), 2.75-2.64 (1H, m), 2.55 (3H, app. s), 2.03-1.87 (1H, m), 1.49 (9H, s).

### Step 6 (Scheme 17) leading to Example 202: (S)-N-(2,3-dihydro-1H-inden-1-yl)-4-methyl-2-(piperazin-1-yl)benzo[d]thiazole-6-carboxamide

To a solution of tert-butyl (S)-4-(6-((2,3-dihydro-1H-inden-1-yl)carbamoyl)-4-methylbenzo[d]thiazol-2-yl)piperazine-1-carboxylate (1.00 equiv.) in MeOH (0.21 M solution of substrate) was added 4 M HCl/dioxane (56.8 equiv. HCl). The resulting slurry was sonicated for 15 min at ambient temperature and then evaporated to dryness to afford a solid residue that was partitioned between NaOH solution (1 volume) and 2% v/v MeOH/DCM (5 volumes).

The separated organic phase was dried (Na₂SO₄), filtered through Celite® and evaporated to dryness to afford (S)-N-(2,3-dihydro-1H-inden-1-yl)-4-methyl-2-(piperazin-1-yl)benzo[d]thiazole-6-carboxamide as a white powder (96% yield).

¹H NMR (as free base): δ_{H} (300 MHz, CDCl₃) 7.96 (1H, dq, J 1.8 and 0.5), 7.48 (1H, dq, J 1.8 and 0.8), 7.40-7.34 (1H, m), 7.30-7.19 (3H, m), 6.35 (1H, br. d, J 8.3), 5.70 (1H, app. q, J 7.7), 3.65-3.62 (4H, m), 3.04 (1H, tented ddd, J 15.9, 8.8 and 4.1), 3.01-2.97 (4H, m), 2.92 (1H, tented dt, J 15.9 and 8.0), 2.70 (1H, dtd, J 12.7, 7.8 and 4.3), 2.55 (3H, app. s), 1.93 (1H, dddd, J 12.9, 8.7, 8.1 and 7.2), 1.74 (1H, br. s).

### Example 203: (S)-N-(2,3-dihydro-1H-inden-1-yl)-2-(4-ethylpiperazin-1-yl)-4-methylbenzo[d]thiazole-6-carboxamide

Example 203 may be prepared according to the illustrative route shown in Scheme 18.

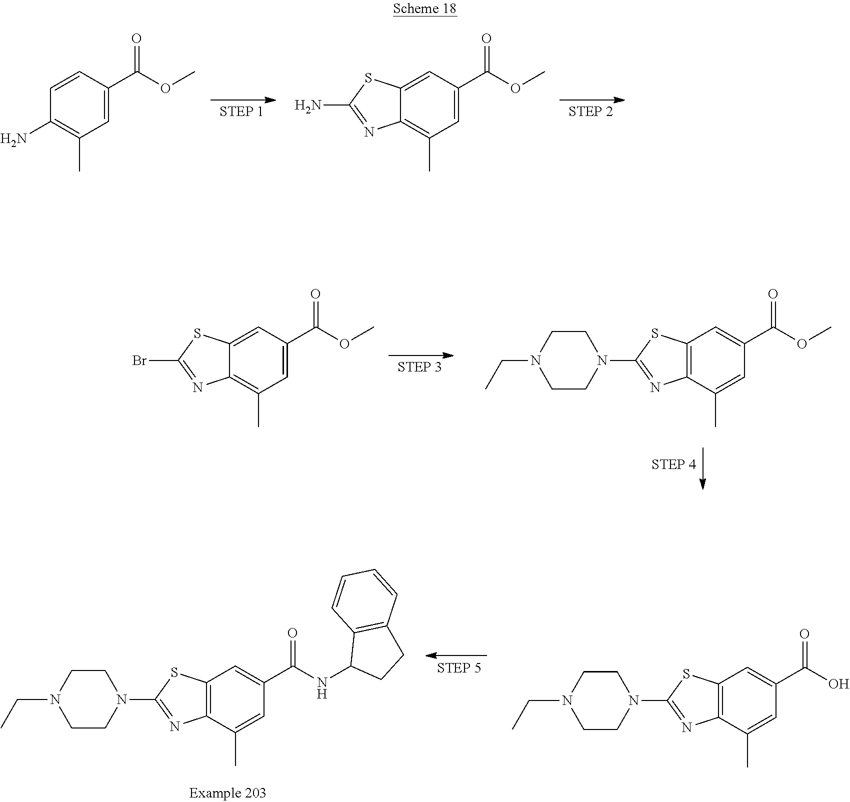 [SMILES: COC(=O)c1ccc(N)c(C)c1 ; COC(=O)c1cc(C)c2nc(N)sc2c1 ; COC(=O)c1cc(C)c2nc(Br)sc2c1 ; CCN1CCN(c2nc3c(C)cc(C(=O)OC)cc3s2)CC1 ; CCN1CCN(c2nc3c(C)cc(C(=O)NC4CCc5ccccc54)cc3s2)CC1 ; CCN1CCN(c2nc3c(C)cc(C(=O)O)cc3s2)CC1]

### Step 1 (Scheme 18): Preparation of 2-amino-4-methylbenzo[d]thiazole-6-carboxylate

As described for Examples 179 and 180 above (Scheme 16).

### Step 2 (Scheme 18): Preparation of 2-bromo-4-methylbenzo[d]thiazole-6-carboxylate

As described for Examples 179 and 180 above (Scheme 16).

### Step 3 (Scheme 18): Preparation of methyl 2-(4-ethylpiperazin-1-yl)-4-methymethyl[d]thiazole-6-carboxylate

A stirred mixture of methyl 2-bromo-4-methylbenzo[d]thiazole-6-carboxylate (1.00 equiv.), powdered K₂CO₃ (2.00 equiv.) and 1-ethylpiperazine (1.11 equiv.) in MeCN (1 volume; 0.075 M solution of substrate) was heated under Ar at 88° C. (sealed pressure vessel). After 16 h, TLC analysis (10% v/v MeOH/DCM) indicated complete conversion of substrate (R_{f} 0.94) to product (R_{f} 0.25). The mixture was cooled and filtered through Celite®, washing with EtOAc (2.5 volumes). The filtrate was washed successively with citric acid solution (0.01 M aq; 2×1 volume) and saturated NaHCO₃ solution (1 volume). The organic phase was dried (Na₂SO₄) and evaporated to afford a residue that was processed chromatographically (silica gel; 2% v/v MeOH/DCM elution). Fractions containing the clean target material were combined and evaporated to afford methyl 2-(4-ethylpiperazin-1-yl)-4-methylbenzo[d]thiazole-6-carboxylate as a pale yellow solid (87% yield).

¹H NMR: δ_{H} (300 MHz, CDCl₃) 8.15 (1H, dq, J 1.7 and 0.5), 7.81 (1H, dq, J 1.7 and 0.8), 3.90 (3H, s), 3.73-3.69 (4H, m), 2.61-2.56 (4H, m), 2.56 (3H, app. s), 2.49 (2H, q, J 7.2), 1.13 (3H, t, J 7.2).

### Step 4 (Scheme 18): Preparation of 2-(4-ethylpiperazin-1-yl)-4-methylbenzo[d]thiazole-6-carboxylic acid

To a stirred solution of methyl 2-(4-ethylpiperazin-1-yl)-4-methylbenzo[d]thiazole-6-carboxylate (1.00 equiv.) in a 2:2:1 v/v/v mixture of THF, MeOH and water (0.05 M solution of substrate) was added LiOH (4.99 equiv.). The mixture was stirred at ambient temperature for 48 h. It was then concentrated in vacuo to ca. 20% volume, chilled to near 0° C. and acidified to pH 3-4 with hydrochloric acid to deposit a solid. The precipitate was collected by filtration, washing with ice-water, and dried (initially in an oven at 80° C. and then in vacuo over P₂O₅) to afford 2-(4-ethylpiperazin-1-yl)-4-methylbenzo[d]thiazole-6-carboxylic acid as a white solid (quant. yield). 1H NMR: δ_{H} (300 MHz, DMSO-d₆) 11.04 (1H, br. s), 8.25 (1H, dq, J 1.7 and 0.5), 7.73 (1H, dq, J 1.7 and 0.8), 3.16 (2H, q, J7.2), 2.50 (3H, app. s, (overlapping CD₃SOCD₂H solvent signal)), 1.28 (3H, t, J 7.2). (Piperazine methylene resonances were masked by the water signal.)

### Step 5 (Scheme 18) leading to Example 203: (S)-N-(2,3-dihydro-1H-inden-1-yl)-2-(4-ethylpiperazin-1-yl)-4-methylbenzo[d]thiazole-6-carboxamide

To a stirred solution of 2-(4-ethylpiperazin-1-yl)-4-methylbenzo[d]thiazole-6-carboxylic acid (1.00 equiv.), N-(3-dimethylaminopropyl)-N′-ethylcarbodiimide hydrochloride (EDCI·HCl; 1.20 equiv.) and N-hydroxybenzotriazole (HOBt; 1.20 equiv.) in DMF (0.08 M solution of substrate) was added Et₃N (3.23 equiv.) followed after 20 min by (S)-1-aminoindane (1.19 equiv.). After stirring for 16 h at ambient temperature TLC analysis (10% v/v MeOH/DCM) indicated conversion of starting material (R_{f} 0.20) to product (R_{f} 0.36). The mixture was evaporated to dryness (60° C. at 7 mbar) to afford a residue that was taken up in 5% MeOH/CHCl₃ (1 volume) and washed successively with citric acid solution (0.01 M aq; 0.125 volumes), saturated NaHCO₃ solution (0.2 volumes) and brine (0.2 volumes). The organic phase was dried (MgSO₄) and evaporated to afford a buff-coloured solid that was processed chromatographically (silica gel; 0.5-1.0% v/v MeOH/DCM gradient elution). Fractions containing clean target material were combined and evaporated to afford (S)-N-(2,3-dihydro-1H-inden-1-yl)-2-(4-ethylpiperazin-1-yl)-4-methylbenzo[d]thiazole-6-carboxamide as a pale yellow solid (65% yield).

¹H NMR (as free base): δ_{H} (300 MHz, CDCl₃) 7.96 (1H, dq, J 1.8 and 0.5), 7.48 (1H, dq, J 1.8 and 0.8), 7.39-7.34 (1H, m), 7.30-7.19 (3H, m), 6.32 (1H, br. d, J 8.3), 5.70 (1H, app. q, J 7.7), 3.72-3.66 (4H, m), 3.04 (1H, tented ddd, J 15.9, 8.8 and 4.1), 2.92 (1H, dt, J 16.0 and 8.0), 2.70 (1H, dtd, J 12.7, 7.8 and 4.3), 2.61-2.55 (4H, m), 2.55 (3H, app. s), 2.49 (2H, q, J 7.2), 1.93 (1H, dddd, J 12.9, 8.7, 8.1 and 7.2), 1.13 (3H, t, J7.2).

### Example 204: N-cyclobutyl-4-methyl-2-(piperazin-1-yl)benzo[d]thiazole-6-carboxamide

Example 204 may be prepared by analogy to Example 202 of Scheme 17 but using cyclobutylamine in Step 5 in place of (S)-1-aminoindane.

¹H NMR (as free base): δ_{H} (300 MHz, CDCl₃) 7.92 (1H, app. d, J 1.8), 7.46 (1H, dq, J 1.8 and 0.8), 6.20 (1H, br. d, J7.9), 4.41 (1H, app. sextet, J 8.1), 3.66-3.62 (4H, m), 3.03-2.99 (4H, m), 2.56 (3H, app. s), 2.49-2.38 (2H, m), 2.04-1.88 (2H, m), 1.82-1.70 (2H, m), 1.67 (1H, br. s).

### Example 205: N-cyclobutyl-4-methyl-2-(4-methylpiperazin-1-yl)benzo[d]thiazole-6-carboxamide

Example 205 may be prepared by reductive amination of Example 204 with formaldehyde as follows. To a stirred solution of N-cyclobutyl-4-methyl-2-(piperazin-1-yl)benzo[d]thiazole-6-carboxamide (Example 204; 1.0 equiv.) in MeOH (1 volume; 0.1 M in substrate) was added formaldehyde (37% w/w in H₂O; 1.1 equiv.) and acetic acid (4.0 equiv.). The mixture was stirred at ambient temperature for 2 h and then cooled to 0° C. prior to addition of sodium cyanoborohydride (2.0 equiv.), stirring for a further period of 2 h. TLC analysis (10% v/v MeOH/DCM) revealed complete and clean conversion of substrate (R_{f} 0.16) to a faster running product component (R_{f} 0.25). The reaction mixture was quenched with ice-cold water (1.25 volumes), concentrated under reduced pressure and extracted with CHCl₃ (5 volumes). The CHCl₃ extract was washed with saturated NaHCO₃ solution (1.25 volumes) followed by brine (1.25 volumes), dried (Na₂SO₄) and evaporated to dryness under reduced pressure to afford N-cyclobutyl-4-methyl-2-(4-methylpiperazin-1-yl)benzo[d]thiazole-6-carboxamide as a white powder (87% yield).

¹H NMR (as free base): δ_{H} (300 MHz, CDCl₃) 7.92 (1H, dq, J 1.7 and 0.4), 7.46 (1H, dq, J 1.7 and 0.8), 6.21 (1H, br. d, J 8.0), 4.23 (1H, sextet, J 8.1), 3.70-3.67 (4H, m), 2.56 (3H, app. s), 2.56-2.52 (4H, m), 2.49-2.38 (2H, m), 2.36 (3H, s), 2.04-1.88 (2H, m), 1.82-1.70 (2H, m).

### Example 206: N-cyclopentyl-4-methyl-2-(piperazin-1-yl)benzo[d]thiazole-6-carboxamide

Example 206 may be prepared by analogy to Example 202 of Scheme 17 but using cyclopentylamine in Step 5 in place of (S)-1-aminoindane.

¹H NMR (as free base): δ_{H} (300 MHz, CDCl₃) 7.91 (1H, app. d, J 1.7), 7.45 (1H, dq, J 1.7 and 0.8), 6.01 (1H, br. d, J 7.0), 4.41 (1H, app. sextet, J 7.0), 3.66-3.62 (4H, m), 3.02-2.99 (4H, m), 2.56 (3H, app. s), 2.15-2.05 (2H, m), 1.80-1.59 (5H, m), 1.56-1.43 (2H, m).

### Example 207: N-cyclopentyl-2-(4-ethylpiperazin-1-yl)-4-methylbenzo[d]thiazole-6-carboxamide

Example 207 may be prepared by analogy to Example 203 of Scheme 18 but using cyclopentylamine in Step 5 in place of (S)-1-aminoindane.

¹H NMR (as free base): δ_{H} (300 MHz, CDCl₃) 7.91 (1H, dq, J 1.8 and 0.4), 7.44 (1H, dq, J 1.8 and 0.8), 5.99 (1H, br. d, J 7.6), 4.41 (1H, app. sextet, J 7.0), 3.72-3.66 (4H, m), 2.61-2.57 (4H, m), 2.57 (3H, app. s), 2.49 (2H, q, J 7.2), 2.15-2.04 (2H, m), 1.79-1.59 (4H, m), 1.55-1.44 (2H, m), 1.13 (3H, t, J7.2).

### Example 208: N-cyclopentyl-4-methyl-2-(4-methylpiperazin-1-yl)benzo[d]thiazole-6-carboxamide

Example 208 may be prepared by reductive amination of Example 206 with formaldehyde as follows. To a stirred solution of N-cyclopentyl-4-methyl-2-(piperazin-1-yl)benzo[d]thiazole-6-carboxamide (Example 206; 1.0 equiv.) in MeOH (1 volume; 0.1 M in substrate) was added formaldehyde (37% w/w in H₂O; 1.1 equiv.) and acetic acid (4.0 equiv.). The mixture was stirred at ambient temperature for 2 h and then cooled to 0° C. prior to addition of sodium cyanoborohydride (2.0 equiv.), stirring for a further period of 2 h. TLC analysis (10% v/v MeOH/DCM) revealed complete and clean conversion of substrate (R_{f} 0.14) to a faster running product component (R_{f} 0.24). The reaction mixture was quenched with ice-cold water (1 volume), concentrated under reduced pressure and extracted with CHCl₃ (5 volumes). The CHCl₃ extract was washed with saturated NaHCO₃ solution (1 volume) followed by brine (1 volume), dried (Na₂SO₄) and evaporated to dryness under reduced pressure to afford N-cyclopentyl-4-methyl-2-(4-methylpiperazin-1-yl)benzo[d]thiazole-6-carboxamide as a white powder (93% yield).

¹H NMR (as free base): δ_{H} (300 MHz, CDCl₃) 7.91 (1H, dq, J 1.7 and 0.4), 7.44 (1H, dq, J1.7 and 0.8), 6.21 (1H, br. d, J7.1), 4.41 (1H, sextet, J7.0), 3.70-3.67 (4H, m), 2.56 (3H, app. s), 2.56-2.53 (4H, m), 2.36 (3H, s), 2.17-2.03 (2H, m), 1.81-1.57 (4H, m), 1.57-1.43 (2H, m).

### Example 209: 4-methyl-2-(piperazin-1-yl)-N-(tetrahydro-2H-pyran-4-yl)benzo[d]thiazole-6-carboxamide

Example 209 may be prepared by analogy to Example 202 of Scheme 17 but using tetrahydro-2H-pyran-4-amine in Step 5 in place of (S)-1-aminoindane.

¹H NMR (as free base): δ_{H} (300 MHz, CDCl₃) 7.92 (1H, dq, J 1.7 and 0.5), 7.46 (1H, dq, J 1.7 and 0.8), 5.99 (1H, br. d, J 7.7), 4.21 (1H, tdt, J 11.3, 7.6 and 3.8), 4.05-3.94 (2H, m), 3.66-3.62 (4H, m), 3.53 (2H, td, J 11.7 and 2.0), 3.02-2.98 (4H, m), 2.56 (3H, app. s), 2.05-1.96 (2H, m), 1.70 (1H, br. s), 1.64-1.51 (2H, m).

### Example 210: 2-(4-ethylpiperazin-1-yl)-4-methyl-N-(tetrahydro-2H-pyran-4-yl)benzo[d]thiazole-6-carboxamide

Example 210 may be prepared by analogy to Example 203 of Scheme 18 but using tetrahydro-2H-pyran-4-amine in Step 5 in place of (S)-1-aminoindane.

¹H NMR (as free base): δ_{H} (300 MHz, CDCl₃) 7.92 (1H, dq, J 1.8 and 0.5), 7.46 (1H, dq, J 1.8 and 0.8), 5.95 (1H, br. d, J7.8), 4.28-4.15 (1H, m), 4.04-3.96 (2H, m), 3.73-3.67 (4H, m), 3.54 (2H, td, J 11.7 and 2.1), 2.61-2.57 (4H, m), 2.57 (3H, app. s), 2.49 (2H, q, J7.2), 2.06-1.97 (2H, m), 1.51-1.64-1.51 (2H, m), 1.13 (3H, t, J 7.2).

### Example 211: (S)-N-(chroman-4-yl)-4-methyl-2-(piperazin-1-yl)benzo[d]thiazole-6-carboxamide

Example 211 may be prepared by analogy to Example 202 of Scheme 17 but using (S)-chroman-4-amine in Step 5 in place of (S)-1-aminoindane.

¹H NMR (as free base): δ_{H} (300 MHz, CDCl₃) 7.93 (1H, dq, J 1.8 and 0.5), 7.47 (1H, dq, J1.8 and 0.8), 7.27 (1H, dd, J7.9 and 1.5), 7.22-7.16 (1H, m), 6.91 (1H, td, J7.5 and 1.2), 6.85 (1H, dd, J 8.2 and 1.2), 6.47 (1H, br. d, J7.4), 5.32 (1H, dt, J7.4 and 5.3), 4.29 (1H, tented ddd, J 11.5, 6.0 and 3.6), 4.21 (1H, tented ddd, J 11.5, 8.6 and 3.0), 3.65-3.59 (4H, m), 3.01-2.96 (4H, m), 2.54 (3H, app. s), 2.29 (1H, tented dddd, J 14.1, 8.8, 5.1 and 3.7), 2.17 (1H, tented dtd, J 14.1, 5.7 and 3.0), 1.78 (1H, br. s).

### Example 212: (S)-N-(chroman-4-yl)-2-(4-ethylpiperazin-1-yl)-4-methylbenzo[d]thiazole-6-carboxamide

Example 212 may be prepared by analogy to Example 203 of Scheme 18 but using (S)-chroman-4-amine in Step 5 in place of (S)-1-aminoindane.

¹H NMR (as free base): δ_{H} (300 MHz, CDCl₃) 7.95 (1H, dq, J 1.8 and 0.5), 7.48 (1H, dq, J 1.8 and 0.8), 7.29 (1H, app. dd, J 7.7 and 1.3), 7.24-7.18 (1H, m), 6.92 (1H, td, J 7.5 and 1.2), 6.87 (1H, dd, J 8.2 and 1.1), 6.36 (1H, br. d, J 7.4), 5.34 (1H, dt, J 7.2 and 5.3), 4.31 (1H, tented ddd, J 11.5, 6.0 and 3.5), 4.22 (1H, ddd, J 11.5, 8.7 and 2.9), 3.76-3.69 (4H, m), 2.65-2.59 (4H, m), 2.55 (3H, app, s), 2.53 (2H, q, J 7.2), 2.31 (1H, tented dddd, J 14.0, 8.8, 5.1 and 3.7), 2.19 (1H, tented dtd, J14.1, 5.7 and 2.9), 1.16 (3H, t, J7.2).

### Example 213: N-cyclopentyl-5-methyl-2-(piperazin-1-yl)benzo[d]thiazole-6-carboxamide

Example 213 may be prepared by analogy to Example 202 of Scheme 17 but commencing with methyl 4-amino-2-methylbenzoate in Step 1 instead of methyl 4-amino-3-methylbenzoate and using cyclopentylamine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.59 (1H, s), 7.35 (1H, s), 5.72 (1H, br. d, J7.6), 4.40 (1H, app. sextet, J 6.8), 3.63-3.57 (4H, m), 3.02-2.97 (4H, m), 2.48 (3H, s), 2.14-2.02 (2H, m), 1.73-1.62 (5H, m), 1.54-1.42 (2H, m).

### Example 214: N-cyclopentyl-2-(4-ethylpiperazin-1-yl)-5-methylbenzo[d]thiazole-6-carboxamide

Example 214 may be prepared by analogy to Example 203 of Scheme 18 but commencing with methyl 4-amino-2-methylbenzoate in Step 1 instead of methyl 4-amino-3-methylbenzoate and using cyclopentylamine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.59 (1H, s), 7.35 (1H, s), 5.70 (1H, br. d, J7.6), 4.39 (1H, app. sextet, J 6.9), 3.70-3.62 (4H, m), 2.61-2.54 (4H, m), 2.48 [(2H, q, J 7.2) and overlapping (3H, s)], 2.13-2.03 (2H, m), 1.76-1.61 (4H, m), 1.54-1.43 (2H, m), 1.12 (3H, t, J7.2).

### Example 215: (S)-N-(chroman-4-yl)-5-methyl-2-(piperazin-1-yl)benzo[d]thiazole-6-carboxamide

Example 215 may be prepared by analogy to Example 202 of Scheme 17 but commencing with methyl 4-amino-2-methylbenzoate in Step 1 instead of methyl 4-amino-3-methylbenzoate and using (S)-chroman-4-amine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.63 (1H, s), 7.37 (1H, s), 7.31 (1H, app. dd, J 7.7 and 1.1), 7.23-7.17 (1H, m), 6.93 (1H, td, J 7.5 and 1.1), 6.85 (1H, dd, J 8.3 and 1.0), 6.12 (1H, br. d, J7.7), 5.34 (1H, dt, J7.5 and 5.4), 4.33 (1H, tented ddd, J 11.5, 6.0 and 3.5), 4.20 (1H, tented ddd, J 11.5, 8.9 and 2.7), 3.64-3.54 (4H, m), 3.02-2.94 (4H, m), 2.54 (3H, s), 2.35 (1H, tented dddd, J 14.0, 8.8, 5.1 and 3.7), 2.19 (1H, tented dtd, J 14.1, 5.7 and 2.9), 1.64 (1H, br. s).

### Example 216: (S)-N-(chroman-4-yl)-2-(4-ethylpiperazin-1-yl)-5-methylbenzo[d]thiazole-6-carboxamide

Example 216 may be prepared by analogy to Example 203 of Scheme 18 but commencing with methyl 4-amino-2-methylbenzoate in Step 1 instead of methyl 4-amino-3-methylbenzoate and using (S)-chroman-4-amine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.62 (1H, s), 7.35 (1H, s), 7.30 (1H, app. dd, J 7.6 and 1.2), 7.22-7.16 (1H, m), 6.92 (1H, td, J7.5 and 1.2), 6.84 (1H, dd, J 8.2 and 1.2), 6.08 (1H, br. d, J 7.7), 5.33 (1H, dt, J 7.5 and 5.4), 4.32 (1H, tented ddd, J 11.5, 5.9 and 3.5), 4.19 (1H, tented ddd, J 11.5, 8.9 and 2.7), 3.68-3.62 (4H, m), 2.59-2.53 (4H, m), 2.53 (3H, s), 2.47 (2H, q, J7.2), 2.33 (1H, tented dddd, J 14.1, 8.9, 5.2 and 3.6), 2.18 (1H, tented dtd, J 14.1, 5.8 and 2.7), 1.12 (3H, t, J7.2).

### Example 217: N-cyclopentyl-7-methyl-2-(piperazin-1-yl)benzo[d]thiazole-6-carboxamide

Example 217 may be prepared by analogy to Example 202 of Scheme 17 but commencing with methyl 4-amino-2-methylbenzoate in Step 1 instead of methyl 4-amino-3-methylbenzoate and using cyclopentylamine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.32 and 7.29 (2H, tight AB spin system, J 8.3), 5.83 (1H, br. d, J7.4), 4.37 (1H, app. sextet, J6.9), 3.64-3.57 (4H, m), 3.01-2.94 (4H, m), 2.53 (3H, s), 2.12-2.01 (2H, m), 1.74 (1H, br. s), 1.72-1.57 (4H, m), 1.53-1.40 (2H, m).

### Example 218: N-cyclopentyl-2-(4-ethylpiperazin-1-yl)-7-methylbenzo[d]thiazole-6-carboxamide

Example 218 may be prepared by analogy to Example 203 of Scheme 18 but commencing with methyl 4-amino-2-methylbenzoate in Step 1 instead of methyl 4-amino-3-methylbenzoate and using cyclopentylamine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.34 and 7.31 (2H, tight AB spin system, J8.3), 5.75 (1H, br. d, J 7.5), 4.39 (1H, app. sextet, J 6.8), 3.72-3.64 (4H, m), 2.61-2.55 (4H, m), 2.55 (3H, s), 2.48 (2H, q, J7.2), 2.14-2.03 (2H, m), 1.76-1.58 (4H, m), 1.56-1.42 (2H, m), 1.12 (3H, t, J7.2).

### Example 219: (S)-N-(chroman-4-yl)-7-methyl-2-(piperazin-1-yl)benzo[d]thiazole-6-carboxamide

Example 219 may be prepared by analogy to Example 202 of Scheme 17 but commencing with methyl 4-amino-2-methylbenzoate in Step 1 instead of methyl 4-amino-3-methylbenzoate and using (S)-chroman-4-amine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.33 and 7.31 (2H, tight AB spin system, J 8.3), 7.29 (1H, app. dd, J7.7 and 1.1), 7.21-7.15 (1H, m), 6.91 (1H, td, J7.5 and 1.2), 6.83 (1H, dd, J 8.2 and 1.2), 6.27 (1H, br. d, J7.7), 5.31 (1H, dt, J7.5 and 5.4), 4.31 (1H, tented ddd, J 11.5, 6.2 and 3.4), 4.19 (1H, tented ddd, J 11.5, 8.8 and 2.8), 3.62-3.55 (4H, m), 2.99-2.92 (4H, m), 2.59 (3H, s), 2.32 (1H, tented dddd, J 14.1, 8.9, 5.2 and 3.6), 2.16 (1H, tented dtd, J 14.1, 5.8 and 2.7), 1.72 (1H, br. s).

### Example 220: (S)-N-(chroman-4-yl)-2-(4-ethylpiperazin-1-yl)-7-methylbenzo[d]thiazole-6-carboxamide

Example 220 may be prepared by analogy to Example 203 of Scheme 18 but commencing with methyl 4-amino-2-methylbenzoate in Step 1 instead of methyl 4-amino-3-methylbenzoate and using (S)-chroman-4-amine in Step 5 in place of (S)-1-aminoindane. Step 1 generates a mixture of methyl 2-amino-5-methylbenzo[d]thiazole-6-carboxylate and methyl 2-amino-7-methylbenzo[d]thiazole-6-carboxylate isomers in 1:1.1 ratio. This mixture may be carried through the route with isomer separation achieved subsequently by chromatography.

¹H NMR (as free base): δ_{H} (300 MHz, CDCl₃) 7.36 and 7.33 (2H, tight AB spin system, J8.3), 7.30 (1H, app. dd, J7.8 and 1.3), 7.22-7.16 (1H, m), 6.92 (1H, td, J7.5 and 1.2), 6.84 (1H, dd, J8.2 and 1.2), 6.12 (1H, br. d, J7.7), 5.33 (1H, dt, J7.5 and 5.4), 4.32 (1H, tented ddd, J 11.5, J6.2 and 3.3), 4.20 (1H, tented ddd, J 11.5, 8.8 and 2.8), 3.71-3.65 (4H, m), 2.61 (3H, s), 2.60-2.55 (4H, m), 2.48 (2H, q, J 7.2), 2.33 (1H, tented dddd, J 14.1, 8.9, 5.2 and 3.6), 2.18 (1H, tented dtd, J 14.2, 5.8 and 2.7), 1.12 (3H, t, J7.2).

### Example 221: N-cyclopentyl-4-methoxy-2-(piperazin-1-yl)benzo[d]thiazole-6-carboxamide

Example 221 may be prepared by analogy to Example 202 of Scheme 17 but commencing with methyl 4-amino-3-methoxybenzoate in Step 1 instead of methyl 4-amino-3-methylbenzoate and using cyclopentylamine in Step 5 in place of (S)-1-aminoindane.

¹H NMR (as free base): δ_{H} (300 MHz, CDCl₃) 7.56 (1H, d, J 1.6), 7.29 (1H, d, J 1.6), 6.01 (1H, br. d, J 7.0), 4.39 (1H, app. sextet, J 6.9), 4.02 (3H, s), 3.67-3.62 (4H, m), 3.02-2.97 (4H, m), 2.16-2.05 (2H, m), 1.80-1.59 (5H, m), 1.56-1.44 (2H, m).

### Example 222: N-cyclopentyl-2-(4-ethylpiperazin-1-yl)-4-methoxybenzo[d]thiazole-6-carboxamide

Example 222 may be prepared by analogy to Example 203 of Scheme 18 but commencing with methyl 4-amino-3-methoxybenzoate in Step 1 instead of methyl 4-amino-3-methylbenzoate and using cyclopentylamine in Step 5 in place of (S)-1-aminoindane.

¹H NMR (as free base): δ_{H} (300 MHz, CDCl₃) 7.59 (1H, d, J 1.6), 7.32 (1H, d, J 1.6), 6.04 (1H, br. d, J 7.1), 4.44 (1H, app. sextet, J 7.0), 4.06 (3H, s), 3.77-3.69 (4H, m), 2.64-2.56 (4H, m), 2.51 (2H, q, J 7.2), 2.20-2.09 (2H, m), 1.83-1.62 (4H, m), 1.59-1.47 (2H, m), 1.16 (3H, t, J 7.2).

### Example 223: (S)-N-(chroman-4-yl)-5-methyl-2-(piperazin-1-yl)benzo[d]thiazole-6-carboxamide

Example 223 may be prepared by analogy to Example 202 of Scheme 17 but commencing with methyl 4-amino-3-methoxybenzoate in Step 1 instead of methyl 4-amino-3-methylbenzoate and using (S)-chroman-4-amine in Step 5 in place of (S)-1-aminoindane.

¹H NMR (as free base): δ_{H} (300 MHz, CDCl₃) 7.60 (1H, d, J 1.6), 7.32 (1H, d, J 1.6), 7.29 (1H, app. dd, J8.0 and 1.2), 7.19-7.25 (1H, m), 6.93 (1H, td, J7.5 and 1.2), 6.88 (1H, dd, J8.2 and 1.2), 6.33 (1H, br. d, J 7.3), 5.35 (1H, dt, J 7.2 and 5.4), 4.32 (1H, tented ddd, J 11.5, 6.2 and 3.3), 4.22 (1H, tented ddd, J 11.5, 8.8 and 2.9), 4.03 (3H, s), 3.68-3.62 (4H, m), 3.03-2.97 (4H, m), 2.33 (1H, tented dddd, J 14.0, 8.8, 5.1 and 3.6), 2.20 (1H, tented dtd, J 14.1, 5.7 and 2.8), 1.82 (1H, br. s).

### Example 224: (S)-N-(2,3-dihydro-1H-inden-1-yl)-5-methoxy-2-(piperazin-1-yl)benzo[d]thiazole-6-carboxamide

Example 224 may be prepared by analogy to Example 202 of Scheme 17 but commencing with methyl 4-amino-2-methoxybenzoate in Step 1 instead of methyl 4-amino-3-methylbenzoate. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.51 (1H, s), 8.19 (1H, br. d, J7.9), 7.41-7.35 (1H, m), 7.30-7.19 (3H, m), 7.11 (1H, s), 5.73 (1H, app. q, J 7.9), 3.89 (3H, s), 3.67-3.62 (4H, m), 3.07-2.87 (6H, m), 2.74 (1H, dtd, J 12.6, 7.5 and 3.3), 1.90 (1H, app. dq, J 12.6 and 8.6), 1.72 (1H, br. s).

### Example 225: N-cyclopentyl-5-methoxy-2-(piperazin-1-yl)benzo[d]thiazole-6-carboxamide

Example 225 may be prepared by analogy to Example 202 of Scheme 17 but commencing with methyl 4-amino-2-methoxybenzoate in Step 1 instead of methyl 4-amino-3-methylbenzoate and using cyclopentylamine in Step 5 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.44 (1H, s), 7.92 (1H, br. d, J7.0), 7.11 (1H, s), 4.42 (1H, app. sextet, J 6.7), 3.96 (3H, s), 3.66-3.60 (4H, m), 3.03-2.97 (4H, m), 2.12-2.01 (2H, m), 1.80-1.60 (5H, m), 1.58-1.46 (2H, m).

### Example 226: N-cyclopentyl-2-(4-ethylpiperazin-1-yl)-5-methoxybenzo[d]thiazole-6-carboxamide

Example 226 may be prepared by analogy to Example 203 of Scheme 18 but commencing with methyl 4-amino-2-methoxybenzoate in Step 1 instead of methyl 4-amino-3-methylbenzoate and using cyclopentylamine in Step 5 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.43 (1H, s), 7.92 (1H, br. d, J7.0), 7.11 (1H, s), 4.42 (1H, app. sextet, J6.7), 3.96 (3H, s), 3.72-3.63 (4H, m), 2.61-2.54 (4H, m), 2.48 (2H, q, J 7.2), 2.13-1.99 (2H, m), 1.79-1.58 (4H, m), 1.58-1.45 (2H, m), 1.12 (3H, t, J 7.2).

### Example 227: 5-methoxy-2-(piperazin-1-yl)-N-(tetrahydro-2H-pyran-4-yl)benzo[d]thiazole-6-carboxamide

Example 227 may be prepared by analogy to Example 202 of Scheme 17 but commencing with methyl 4-amino-2-methoxybenzoate in Step 1 instead of methyl 4-amino-3-methylbenzoate and using tetrahydro-2H-pyran-4-amine in Step 5 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.43 (1H, s), 7.92 (1H, br. d, J7.7), 7.12 (1H, s), 4.24 (1H, tdt, J 10.7, 7.7 and 3.9), 3.98 (3H, s), 3.97 (2H, app. dt, J 11.8 and 3.7), 3.66-3.60 (4H, m), 3.57 (2H, td, J 11.4 and 2.1), 3.03-2.98 (4H, m), 2.08-1.97 (2H, m), 1.69 (1H, br. s), 1.58 (2H, dtd, J 13.1, 10.7 and 4.3).

### Example 228: 2-(4-ethylpiperazin-1-yl)-5-methoxy-N-(tetrahydro-2H-pyran-4-yl)benzo[d]thiazole-6-carboxamide

Example 228 may be prepared by analogy to Example 203 of Scheme 18 but commencing with methyl 4-amino-2-methoxybenzoate in Step 1 instead of methyl 4-amino-3-methylbenzoate and using tetrahydro-2H-pyran-4-amine in Step 5 in place of (S)-1-aminoindane. Methyl 2-amino-5-methoxybenzo[d]thiazole-6-carboxylate is isolated as the sole isomer from Step 1 in essentially quantitative yield; the 7-methoxy isomer is not accessible by this route.

¹H NMR (as free base): δ_{H} (300 MHz, CDCl₃) 8.43 (1H, s), 7.92 (1H, br. d, J7.6), 7.13 (1H, s), 4.24 (1H, tdt, J 10.7, 7.8 and 3.8), 3.97 (3H, s), 3.97 (2H, app. dt, J 11.8 and 3.7), 3.71-3.66 (4H, m), 3.57 (2H, td, J 11.3 and 2.0), 2.60-2.55 (4H, m), 2.48 (2H, q, J 7.2), 2.08-1.98 (2H, m), 1.58 (2H, dtd, J13.1, 10.7 and 4.3), 1.12 (3H, t, J7.2).

### Example 229: (S)-N-(chroman-4-yl)-5-methoxy-2-(piperazin-1-yl)benzo[d]thiazole-6-carboxamide

Example 229 may be prepared by analogy to Example 202 of Scheme 17 but commencing with methyl 4-amino-2-methoxybenzoate in Step 1 instead of methyl 4-amino-3-methylbenzoate and using (S)-chroman-4-amine in Step 5 in place of (S)-1-aminoindane.

¹H NMR (as free base): δ_{H} (300 MHz, CDCl₃) 8.47 (1H, s), 8.17 (1H, br. d, J 7.4), 7.33 (1H, app. d, J7.8), 7.22-7.16 (1H, m), 7.10 (1H, s), 6.91 (1H, td, J7.5 and 1.2), 6.86 (1H, dd, J 8.2 and 1.1), 5.41 (1H, app. q, J 6.5), 4.32 (1H, tented ddd, J 11.3, 7.1 and 3.4), 4.23 (1H, tented ddd, J 11.3, 8.0 and 3.1), 3.87 (3H, s), 3.67-3.61 (4H, m), 3.04-2.98 (4H, m), 2.36 (1H, dddd, J 13.7, 8.3, 5.3 and 3.2), 2.15 (1H, ddt, J 13.8, 6.8 and 3.4), 1.70 (1H, br. s).

### Example 230: (S)-N-(chroman-4-yl)-4,7-dimethyl-2-(piperidin-4-yl)benzo[d]thiazole-6-carboxamide

Example 230 may be prepared by analogy to Example 179 of Scheme 16 but commencing with methyl 4-amino-2,5-dimethylbenzoate in Step 1 instead of methyl 4-amino-3-methylbenzoate and using (S)-chroman-4-amine in place of (S)-1-aminoindane in Step 6.

¹H NMR (as free base): δ_{H} (300 MHz, CDCl₃) 7.32 (1H, dd, J 7.7 and 1.3), 7.27 (1H, s), 7.24-7.18 (1H, m), 6.95 (1H, td, J 7.5 and 1.2), 6.86 (1H, dd, J 8.2 and 1.1), 6.10 (1H, br. d, J 7.3), 5.40-5.34 (1H, m), 4.38-4.31 (1H, m), 4.21 (1H, ddd, J 11.5, 8.9 and 2.7), 3.32-3.19 (3H, m), 2.80 (2H, td, J 12.1 and 2.4), 2.70 (6H, app. s), 2.37 (1H, dddd, J 14.1, 8.9, 5.2 and 3.6), 2.27-2.12 (3H, m), 1.90-1.75 (3H, m).

### Example 231: 4-chloro-N-cyclopentyl-2-(1-methylpiperidin-4-yl)benzo[d]thiazole-6-carboxamide

Example 231 may be prepared by analogy to Example 180 of Scheme 16 but commencing with methyl 4-amino-3-chlorobenzoate in Step 1 instead of methyl 4-amino-3-methylbenzoate, using cyclopentylamine in Step 6 instead of (S)-1-aminoindane, and replacing acetaldehyde with formaldehyde in Step 8.

¹H NMR (as free base): δ_{H} (300 MHz, CDCl₃) 8.21 (1H, d, J1.6), 7.80 (1H, d, J 1.6), 6.04 (1H, br. d, J7.7), 4.42−(1H, app. sextet, J7.1), 3.21 (1H, tt, J11.7 and 3.9), 3.03-2.95 (2H, m), 2.34 (3H, s), 2.27-2.06 (6H, m), 1.95 (2H, qd, J 12.0 and 3.2), 1.82-1.58 (4H, m), 1.57-1.45 (2H, m).

### Example 232: 4-cyclopropyl-N-isopropyl-2-(piperidin-4-yl)benzo[d]thiazole-6-carboxamide

Example 232 may be prepared by analogy to Example 179 of Scheme 16 but commencing with methyl 4-amino-3-cyclopropylbenzoate in Step 1 instead of methyl 4-amino-3-methylbenzoate and using isopropylamine in Step 6 in place of (S)-1-aminoindane.

¹H NMR (as free base): δ_{H} (300 MHz, CDCl₃) 8.01 (1H, d, J 1.7), 7.27 (1H, d, J 1.6), 5.92 (1H, br. d, J7.2), 4.37-4.22 (1H, m), 3.33-3.19 (3H, m), 2.85-2.74 (3H, m), 2.23-2.14 (2H, m), 1.91-1.77 (3H, m), 1.27 (6H, J 6.6), 1.17-1.08 (2H, m), 1.08-0.98 (2H, m).

### Example 233: N-cyclobutyl-4-cyclopropyl-2-(1-methylpiperidin-4-yl)benzo[d]thiazole-6-carboxamide

Example 233 may be prepared by analogy to Example 180 of Scheme 16 but commencing with methyl 4-amino-3-cyclopropylbenzoate in Step 1 instead of methyl 4-amino-3-methylbenzoate, using cyclobutylamine in Step 6 instead of (S)-1-aminoindane, and replacing acetaldehyde with formaldehyde in Step 8.

¹H NMR (as free base): δ_{H} (300 MHz, CDCl₃) 8.02 (1H, d, J 1.7), 7.281 (1H, d, J 1.7), 6.23 (1H, br. d, J7.5), 4.63 (1H, app. sextet, J 8.0), 3.13 (1H, tt, J11.2 and 3.9), 3.03-2.94 (2H, m), 2.78 (1H, tt, J 8.5 and 5.3), 2.50-2.38 (2H, m), 2.34 (3H, s), 2.25-2.08 (4H, m), 2.07-1.90 (4H, m), 1.83-1.72 (2H, m), 1.17-1.08 (2H, m), 1.06-0.98 (2H, m).

### Example 234: 4-chloro-N-cyclobutyl-2-(piperazin-1-yl)benzo[d]thiazole-6-carboxamide

Example 234 may be prepared by analogy to Example 202 of Scheme 17 but commencing with methyl 4-amino-3-chlorobenzoate in Step 1 instead of methyl 4-amino-3-methylbenzoate and using cyclobutylamine in Step 5 instead of 1-aminoindane.

¹H NMR (as free base): δ_{H} (300 MHz, CDCl₃) 7.97 (1H, d, J 1.7), 7.65 (1H, d, J 1.7), 6.18 (1H, br. d, J 7.9), 4.58 (1H, app. sextet, J 8.1), 3.74-3.61 (4H, m), 3.08-2.94 (4H, m), 2.50-2.38 (2H, m), 2.05-1.88 (2H, m), 1.84-1.62 (3H, m).

### Example 235: 4-chloro-N-cyclopentyl-2-(4-(3-hydroxypropyl)piperazin-1-yl)benzo[d]thiazole-6-carboxamide

Example 235 may be prepared according to Scheme 19.

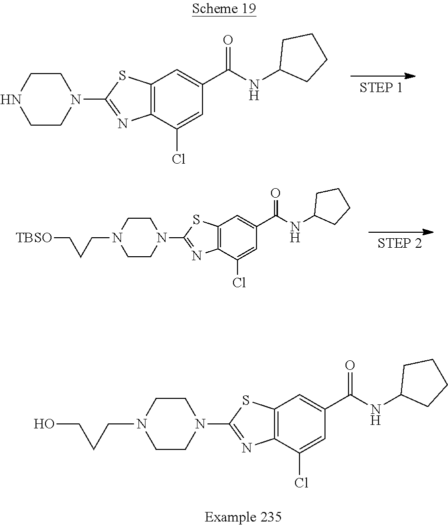 [SMILES: O=C(NC1CCCC1)c1cc(Cl)c2nc(N3CCNCC3)sc2c1 ; CC(C)(C)[Si](C)(C)OCCCN1CCN(c2nc3c(Cl)cc(C(=O)NC4CCCC4)cc3s2)CC1 ; O=C(NC1CCCC1)c1cc(Cl)c2nc(N3CCN(CCCO)CC3)sc2c1]

### Step 1 (Scheme 19): Preparation of 2-(4-(3-((tert-butyldimethylsilyl)oxy)propyl)piperazin-1-yl)-4-chloro-N-cyclopentylbenzo[d]thiazole-6-carboxamide

A stirred mixture of 4-chloro-N-cyclopentyl-2-(piperidin-4-yl)benzo[d]thiazole-6-carboxamide (1.0 equiv.) (prepared by analogy to Example 202 in Scheme 17 but starting from methyl 4-amino-3-chlorobenzoate in Step 1 and using cyclopentylamine in Step 5 in place of 1-aminoindane), DIPEA (2.5 equiv.), (3-bromopropoxy)(tert-butyl)dimethylsilane (2.0 equiv.) and Nal (0.17 equiv.) in MeCN/DMF (9:1 v/v; 0.05 M in substrate) was heated to 84° C. (sealed pressure vessel) for 4 h, and then cooled and concentrated in vacuo. The resulting residue was processed by flash chromatography (0.5-1% v/v MeOH/DCM), and fractions containing the target material were combined and evaporated to afford the title compound as a white powder (70% yield).

¹H NMR: δ_{H} (300 MHz, CDCl₃) 7.99 (1H, d, J 1.7), 7.66 (1H, d, J 1.7), 6.05 (1H, br. d, J7.3), 4.42 (1H, app. sextet, J7.0), 3.80-3.66 (6H, m), 2.68-2.54 (4H, m), 2.52 (2H, t, J7.4), 2.18-2.07 (2H, m), 1.81-1.63 (6H, m), 1.58-1.47 (2H, m), 0.93 (9H, s), 0.09 (6H, s).

### Step 2 (Scheme 19): Preparation of 4-chloro-N-cyclopentyl-2-(4-(3-hydroxypropyl)piperazin-1-yl)benzo[d]thiazole-6-carboxamide

To an ice-cooled, stirred mixture of 2-(4-(3-((tert-butyldimethylsilyl)oxy)propyl)piperazin-1-yl)-4-chloro-N-cyclopentylbenzo[d]thiazole-6-carboxamide (from the preceding step) in pyridine (0.07 M solution of substrate) was added HF/pyridine (70% HF; 39.0 equiv.). The reaction mixture was stirred at ambient temperature for 2 h and then concentrated in vacuo. The resulting residue was diluted with CHCl₃ (1 volume), washed successively with saturated NaHCO₃ solution (0.5 volume) and brine (0.5 volume×2), dried (Na₂SO₄) and filtered through Celite®. The filtrate was evaporated to afford a residue that was triturated with diethyl ether to afford the title compound as a pale yellow solid (66% yield).

¹H NMR (as free base): δ_{H} (300 MHz, CDCl₃) 7.96 (1H, d, J 1.7), 7.64 (1H, d, J 1.7), 6.04 (1H, br. d, J 7.5), 4.45-4.32 (2H, m), 3.83 (2H, t, J 5.3), 3.71 (4H, app. t, J 5.0), 2.74-2.59 (6H, m), 2.16-2.03 (2H, m), 1.83-1.58 (6H, m), 1.58-1.42 (2H, m).

### Example 236: 2-(3,8-diazabicyclo[3.2.1]octan-8-yl)-N-cyclopentyl-7-methoxybenzo[d]thiazole-6-carboxamide

Example 236 may be prepared according to Scheme 20.

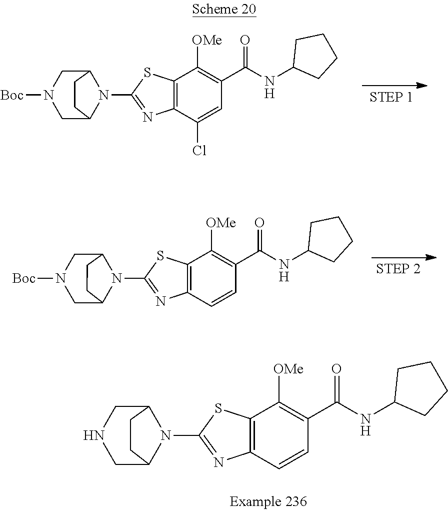 [SMILES: COc1c(C(=O)NC2CCCC2)cc(Cl)c2nc(N3C4CCC3CN(C(=O)OC(C)(C)C)C4)sc12 ; COc1c(C(=O)NC2CCCC2)ccc2nc(N3C4CCC3CN(C(=O)OC(C)(C)C)C4)sc12 ; COc1c(C(=O)NC2CCCC2)ccc2nc(N3C4CCC3CNC4)sc12]

### Step 1 (Scheme 20): Preparation of tert-butyl 8-(6-(cyclopentylcarbamoyl)-7-methoxybenzo[d]thiazol-2-yl)-3,8-diazabicyclo[3.2.1]octane-3-carboxylate

A sealable pressure vessel was purged with argon and charged with a mixture of PdOAc₂ (0.4 equiv.), (2-biphenylyl)di-tert-butylphosphine (0.8 equiv.) and sodium formate (5.0 equiv.) in MeOH (0.02 M in Pd catalyst), stirring at ambient temperature for 20 min. To this mixture was then added a solution of 4-chloro-N-cyclopentyl-2-(piperidin-4-yl)benzo[d]thiazole-6-carboxamide (1.0 equiv.) (prepared according to Steps 1 to 5 of Scheme 17 but starting from methyl 4-amino-5-chloro-2-methoxybenzoate in Step 1, and using tert-butyl 3,8-diazabicyclo[3.2.1]octane-3-carboxylate in Step 3 in place of tert-butyl piperazine-1-carboxylate, and cyclopentylamine in Step 5 in place of 1-aminoindane) in THF (0.1 M in substrate). The reaction mixture was heated to 135° C. for 2.5 h, and then cooled and concentrated in vacuo. The resulting residue was processed by flash chromatography (0.5-1% v/v MeOH/DCM), and fractions containing the target material were combined and evaporated to afford the title compound as a colourless wax (20% yield; 59% yield based on recovered starting material).

¹H NMR: δ_{H} (300 MHz, CDCl₃) 8.08 (1H, d, J 8.5), 7.68 (1H, br. d, J 7.4), 7.40 (1H, d, J 8.5), 4.38-4.55 (3H, m), 4.02-4.01 (1H, m), 3.97 (3H, s), 3.86-3.81 (1H, m), 3.41-3.36 (1H, m), 3.30-3.25 (1H, m), 2.16-2.05 (4H, m), 1.96-1.90 (2H, m), 1.79-1.68 (4H, m), 1.52-1.61-1.52 (2H, m), 1.49 (9H, s).

### Step 2 (Scheme 20): Preparation of 2-(3,8-diazabicyclo[3.2.1]octan-8-yl)-N-cyclopentyl-7-methoxybenzo[d]thiazole-6-carboxamide

Cleavage of the Boc group in the product from the preceding step [tert-butyl 8-(6-(cyclopentylcarbamoyl)-7-methoxybenzo[d]thiazol-2-yl)-3,8-diazabicyclo[3.2.1]octane-3-carboxylate] was carried out as described in Step 6 of Scheme 17 for the preparation of Example 202 and afforded the title compound as a white powder (56% yield).

¹H NMR (as free base): δ_{H} (300 MHz, CDCl₃) 8.03 (1H, d, J 8.6), 7.66 (1H, br. d, J7.3), 7.34 (1H, d, J 8.5), 4.51-4.40 (1H, m), 4.37-4.29 (2H, m), 3.93 (3H, s), 3.25 (2H, dd, J 12.3 and 1.5), 2.78 (2H, dd, J 12.1 and 2.0), 2.22 (1H, br. s), 2.19-1.99 (6H, m), 1.80-1.59 (4H, m), 1.59-1.45 (2H, m).

### Example 237: 2-(3,8-diazabicyclo[3.2.1]octan-8-yl)-4-bromo-N-cyclopentylbenzo[d]thiazole-6-carboxamide

Example 237 may be prepared according to Scheme 21.

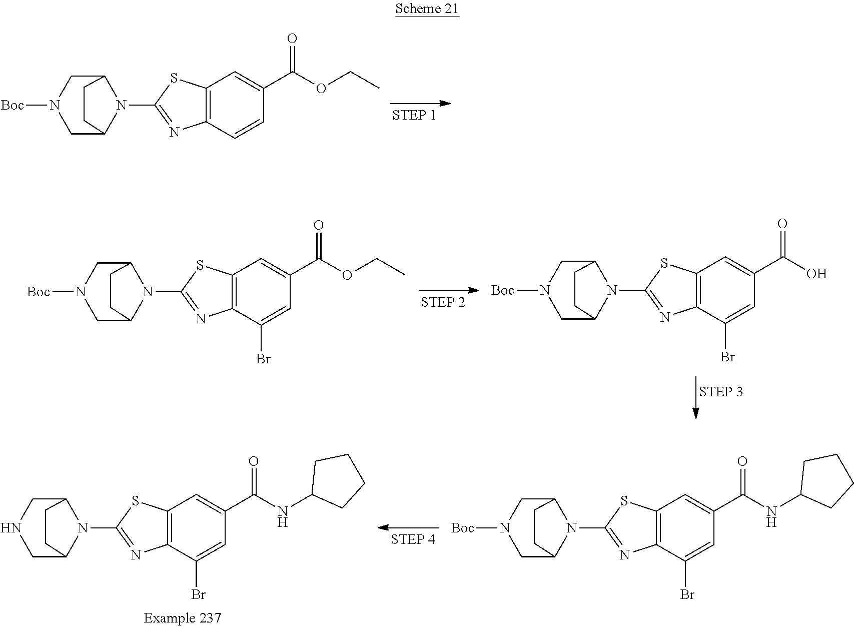 [SMILES: CCOC(=O)c1ccc2nc(N3C4CCC3CN(C(=O)OC(C)(C)C)C4)sc2c1 ; CCOC(=O)c1cc(Br)c2nc(N3C4CCC3CN(C(=O)OC(C)(C)C)C4)sc2c1 ; CC(C)(C)OC(=O)N1CC2CCC(C1)N2c1nc2c(Br)cc(C(=O)O)cc2s1 ; O=C(NC1CCCC1)c1cc(Br)c2nc(N3C4CCC3CNC4)sc2c1 ; CC(C)(C)OC(=O)N1CC2CCC(C1)N2c1nc2c(Br)cc(C(=O)NC3CCCC3)cc2s1]

### Step 1 (Scheme 21): Preparation of ethyl 4-bromo-2-(3-(tert-butoxycarbonyl)-3,8-diazabicyclo[3.2.1]octan-8-yl)benzo[d]thiazole-6-carboxylate

To an argon purged, stirred mixture of ethyl 2-(3-(tert-butoxycarbonyl)-3,8-diazabicyclo[3.2.1]octan-8-yl)benzo[d]thiazole-6-carboxylate (1.0 equiv.) (prepared as in Step 3 of Scheme 17 but from ethyl 2-bromobenzo[d]thiazole-6-carboxylate reacted with tert-butyl 3,8-diazabicyclo[3.2.1]octane-3-carboxylate in place of tert-butyl piperazine-1-carboxylate) and ammonium acetate (0.2 equiv.) in acetonitrile (0.1 M in substrate) was added N-bromosuccinimide (1.4 equiv.). After 18 h at ambient temperature, the solvent was removed in vacuo to afford a residue that was dissolved in EtOAc (1 volume) and washed successively with 1 M NaOH solution (0.5 volume) and brine (0.4 volume). The organic phase was dried (MgSO₄) and evaporated to give the title compound as a buff amorphous solid (94% yield).

¹H NMR δ_{H} (400 MHz, CDCl₃) 8.24 (1H, d, J 1.6), 8.21 (1H, d, J 1.6), 4.51-4.45 (2H, m), 4.40 (2H, q, J 7.1), 4.01 (1H, d, J 13.1), 3.86 (1H, d, J 12.6), 3.39 (1H, d, J 13.0), 3.26 (1H, d, J 12.4), 2.15-2.12 (2H, m), 1.96-1.90 (2H, m), 1.49 (9H, s), 1.42 (3H, t, J7.1).

### Steps 2-4 (Scheme 21): Preparation of 2-(3,8-diazabicyclo[3.2.1]octan-8-yl)-4-bromo-N-cyclopentylbenzo[d]thiazole-6-carboxamide

Ethyl 4-bromo-2-(3-(tert-butoxycarbonyl)-3,8-diazabicyclo[3.2.1]octan-8-yl)benzo[d]thiazole-6-carboxylate from the preceding step was converted into the title compound according to steps 2-4 of Scheme 21 following the procedures described for Steps 4-6 of Scheme 17 but using cyclopentylamine in place of 1-aminoindane.

¹H NMR δ_{H} (400 MHz, CDCl₃) 8.00 (1H, d, J 1.7), 7.78 (1H, d, J 1.7), 5.98 (1H, br. d, J7.0), 4.44-4.30 (3H, m), 3.24 (2H, dd, J12.4 and 1.6), 2.77 (2H, dd, J12.0 and 2.1), 2.16-1.98 (6H, m), 1.79-1.61 (5H, m), 1.54-1.45 (2H, m).

### Example 238: 2-(3,8-diazabicyclo[3.2.1]octan-8-yl)-4-cyano-N-isopropylbenzo[d]thiazole-6-carboxamide

Example 238 may be prepared according to Scheme 22.

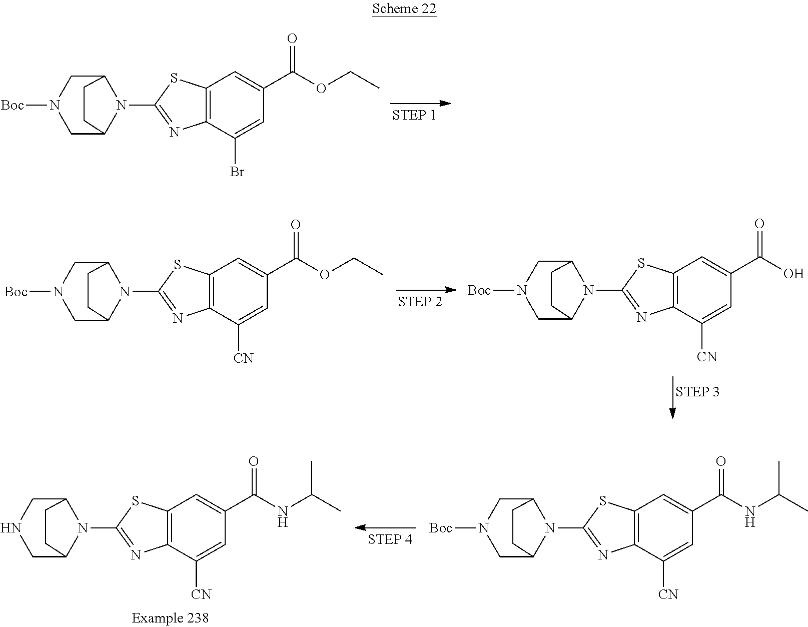 [SMILES: CCOC(=O)c1cc(Br)c2nc(N3C4CCC3CN(C(=O)OC(C)(C)C)C4)sc2c1 ; CCOC(=O)c1cc(C#N)c2nc(N3C4CCC3CN(C(=O)OC(C)(C)C)C4)sc2c1 ; CC(C)(C)OC(=O)N1CC2CCC(C1)N2c1nc2c(C#N)cc(C(=O)O)cc2s1 ; CC(C)NC(=O)c1cc(C#N)c2nc(N3C4CCC3CNC4)sc2c1 ; CC(C)NC(=O)c1cc(C#N)c2nc(N3C4CCC3CN(C(=O)OC(C)(C)C)C4)sc2c1]

### Step 1 (Scheme 22): Synthesis of ethyl 2-(3-(tert-butoxycarbonyl)-3,8-diazabicyclo[3.2.1]octan-8-yl)-4-cyanobenzo[d]thiazole-6-carboxylate

To a stirred solution of ethyl 4-bromo-2-(3-(tert-butoxycarbonyl)-3,8-diazabicyclo[3.2.1]octan-8-yl)benzo[d]thiazole-6-carboxylate (prepared as described in Scheme 21; 2 g, 4.0 mmol, 1 equiv.) in DMF (15 mL) were added K₄Fe(CN)₅ (3.2 g, 9.6 mmol, 2.4 equiv.), K₂CO₃ (1.1 g, 8.0 mmol, 2 equiv.), t-butylxphos (0.33 g, 0.8 mmol, 0.2 equiv.), and Pd(t-butyl-xphos)G1 (0.5 g, 0.8 mmol, 0.2 equiv.) under continuous purging of nitrogen. The resulting mixture was stirred at 130° C. for 16 h. The reaction mixture was concentrated and diluted with ethyl acetate (50 mL) and passed through a celite bed. The filtrate was washed with water (50 mL), brine (50 mL), dried over anhydrous sodium sulfate, filtered and concentrated under reduced pressure to afford crude material (3.5 g) as a gummy solid. The crude compound was processed by flash column chromatography (0-40% EtOAc/petroleum ether) to afford ethyl 2-(3-(tert-butoxycarbonyl)-3,8-diazabicyclo[3.2.1]octan-8-yl)-4-cyanobenzo[d]thiazole-6-carboxylate (850 mg, 44%) as a pale brown solid.

### Step 2 (Scheme 22): Synthesis of 2-(3-(tert-butoxycarbonyl)-3,8-diazabicyclo[3.2.1]octan-8-yl)-4-cyanobenzo[d]thiazole-6-carboxylic acid

To a stirred solution ethyl 2-(3-(tert-butoxycarbonyl)-3,8-diazabicyclo[3.2.1]octan-8-yl)-4-cyanobenzo[d]thiazole-6-carboxylate (700 mg, 1.58 mmol) in THF (14 mL) and water (6 mL) was added LiOH·H₂O (218 mg, 4.75 mmol). The resultant mixture was stirred at r.t. for 16 h.

The reaction mixture was concentrated, diluted with water (10 mL), acidified with citric acid (to pH 4 to 5) and extracted with 10% MeOH/DCM (3×20 mL). The combined organic extract was washed with brine (20 mL), dried over anhydrous sodium sulfate, filtered and concentrated under reduced pressure to afford 700 mg of crude product which was triturated with 10% EtOAc/petroleum ether to afford 2-(3-(tert-butoxycarbonyl)-3,8-diazabicyclo[3.2.1]octan-8-yl)-4-cyanobenzo[d]thiazole-6-carboxylic acid (580 mg, 67%) as a pale brown solid.

### Step 3 (Scheme 22): Synthesis of 2-(3-(tert-butoxycarbonyl)-3,8-diazabicyclo[3.2.1]octan-8-yl)-4-cyano-N-isopropylbenzo[d]thiazole-6-carboxamide

To a stirred solution of 2-(3-(tert-butoxycarbonyl)-3,8-diazabicyclo[3.2.1]octan-8-yl)-4-cyanobenzo[d]thiazole-6-carboxylic acid (150 mg, 0.36 mmol) in DCM (10 mL) at 0° C. was added DIPEA (187 mg, 1.45 mmol) and n-propylphosphonic acid anhydride, cyclic trimer (50% in EtOAc; 230 mg, 0.72 mmol) and the mixture stirred at 0° C. for 15 minutes. Propan-2-amine (107 mg, 1.81 mmol) was added and the mixture stirred at r.t. for 16 h. The mixture was diluted with water (5 mL) and extracted with 10% MeOH/DCM (3×10 mL). The combined organic extract was washed with brine (10 mL), dried over anhydrous sodium sulfate, filtered and concentrated under reduced pressure to afford crude product, which was processed by flash column chromatography (0-100% EtOAc/petroleum ether) to obtain the desired product (110 mg, 55%) as an off-white solid.

### Step 4 (Scheme 22): Synthesis of 2-(3,8-diazabicyclo[3.2.1]octan-8-yl)-4-cyano-N-isopropylbenzo[d]thiazole-6-carboxamide

To a stirred solution of 2-(3-(tert-butoxycarbonyl)-3,8-diazabicyclo[3.2.1]octan-8-yl)-4-cyano-N-isopropylbenzo[d]thiazole-6-carboxamide (110 mg, 0.19 mmol) in DCM (10 mL) at 0° C. was added trifluoroacetic acid (0.073 ml, 0.97 mmol) and the mixture was stirred at r.t. for 16 h.

The mixture was concentrated and the crude product was purified by preparative HPLC using ammonium bicarbonate as buffer to afford the title compound as an off-white solid (35 mg, 50%). ¹H NMR δ_{H} (400 MHz, DMSO-d₆) 8.50 (1H, d, J 1.8), 8.24-8.30 (1H, m), 8.19 (1H, d, J 1.8), 4.19-4.47 (2H, m), 3.95-4.18 (1H, m), 2.86-3.03 (2H, m), 2.65-2.75 (2H, m), 1.95-2.05 (4H, m), 1.17 (6H, d, J 6.6).

### Example 239: 2-(3,8-diazabicyclo[3.2.1]octan-8-yl)-N-cyclopentylbenzo[d]thiazole-6-carboxamide

Example 239 may be prepared by analogy to steps 3 to 6 of Scheme 17 but commencing with ethyl 2-bromobenzo[d]thiazole-6-carboxylate and tert-butyl 3,8-diazabicyclo[3.2.1]octane-3-carboxylate in Step 3, instead of methyl 2-bromo-4-methylbenzo[d]thiazole-6-carboxylate and tert-butyl piperazine-1-carboxylate, and using cyclopentylamine in Step 5 instead of 1-aminoindane. The title compound was isolated as the hydrochloride salt. ¹H NMR δ_{H} (400 MHz, DMSO-d₆) 9.58-9.80 (1H, m), 9.27-9.49 (1H, m), 8.31-8.42 (1H, m), 8.10-8.29 (1H, m), 7.78-7.93 (1H, m), 7.44-7.65 (1H, m), 4.54-4.56 (2H, m), 4.17-4.33 (1H, m), 3.12-3.35 (4H, m), 2.09-2.25 (4H, m), 1.82-1.96 (2H, m), 1.61-1.79 (2H, m), 1.50-1.60 (4H, m).

### Example 240: 2-(3,8-diazabicyclo[3.2.1]octan-8-yl)-N-(4,4-difluorocyclohexyl)-benzo[d]thiazole-6-carboxamide

Example 240 may be prepared by analogy to steps 3 to 6 of Scheme 17 but commencing with ethyl 2-bromobenzo[d]thiazole-6-carboxylate and tert-butyl 3,8-diazabicyclo[3.2.1]octane-3-carboxylate in Step 3, instead of methyl 2-bromo-4-methylbenzo[d]thiazole-6-carboxylate and tert-butyl piperazine-1-carboxylate, and using 4,4-difluorocyclohexylamine in Step 5 instead of 1-aminoindane. The title compound was isolated as the free base. ¹H NMR δ_{H} (400 MHz, DMSO-d₆) 8.25 (1H, d, J 1.6), 8.20 (1H, d, J7.6), 7.78 (1H, dd, J 1.6, 8.4), 7.44 (1H, d, J 8.4), 4.32-4.23 (2H, m), 4.00-3.99 (1H, m), 2.97-2.94 (2H, m), 2.75-2.68 (1H, m), 2.68-2.65 (2H, m), 2.10-1.85 (10H, m), 1.69-1.63 (2H, m).

### Example 241: 2-(3-cyclopropyl-3,8-diazabicyclo[3.2.1]octan-8-yl)-N-(4,4-difluorocyclohexyl)-benzo[d]thiazole-6-carboxamide

Example 241 may be prepared according to Scheme 23.

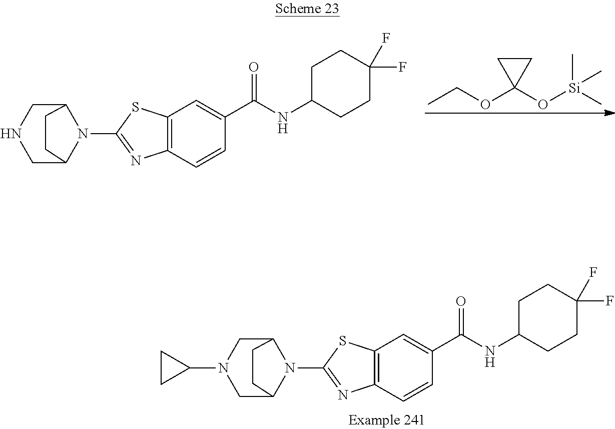

To a stirred solution of 2-(3,8-diazabicyclo[3.2.1]octan-8-yl)-N-(4,4-difluorocyclohexyl) benzo[d]thiazole-6-carboxamide (Example 240; 150 mg, 0.37 mmol) in methanol (5 mL) was added acetic acid (0.042 mL, 0.74 mmol) followed by (1-ethoxycyclopropoxy)trimethylsilane (0.15 ml, 0.74 mmol). The reaction mixture was stirred at r.t. for 2 h, then sodium cyanoborohydride (69.6 mg, 1.11 mmol) was added and the mixture stirred at 60° C. for 16 h. The reaction mixture was concentrated under reduced pressure, diluted with cold water and extracted with 10% methanol in DCM. The organic layer was washed with brine, dried over anhydrous sodium sulfate, filtered and concentrated under reduced pressure. The crude product was purified by flash column chromatography (0-10% methanol in DCM) and further purified by preparative HPLC using ammonium bicarbonate as buffer to afford the title compound (25 mg, 15%) as an off-white solid. ¹H NMR δ_{H} (400 MHz, DMSO-d₆) 8.25-8.28 (1H, m), 8.19-8.25 (1H, m), 7.80 (1H, dd, J 8.4, 1.7), 7.47 (1H, d, J 8.4), 4.30-4.38 (2H, m), 3.87-4.12 (1H, m), 2.71-2.82 (2H, m), 2.56-2.66 (2H, m), 1.72-2.15 (10H, m), 1.60-1.70 (3H, m), 0.38-0.43 (2H, m), 0.29-0.36 (2H, m).

### Example 242: N-(4,4-difluorocyclohexyl)-2-(3-(2-hydroxyethyl)-3,8-diazabicyclo[3.2.1]octan-8-yl]benzo[d]thiazole-6-carboxamide

Example 242 may be prepared by analogy to Example 120 of Scheme 8 but commencing with 2-(3,8-diazabicyclo[3.2.1]octan-8-yl)-N-(4,4-difluorocyclohexyl)benzo[d]thiazole-6-carboxamide (Example 240) instead of (S)-N-(chroman-4-yl)-2-(piperazin-1-yl)benzo[d]thiazole-6-carboxamide hydrochloride. ¹H NMR δ_{H} (400 MHz, DMSO-d₆) 8.24-8.27 (1H, m), 8.19-8.23 (1H, m), 7.70-7.87 (1H, m), 7.36-7.54 (1H, m), 4.26-4.44 (3H, m), 3.92-4.08 (1H, m), 3.42-3.57 (2H, m), 2.71-2.85 (2H, m), 2.35-2.48 (4H, m), 1.80-2.18 (10H, m), 1.53-1.76 (2H, m).

### Example 243: N-(4,4-difluorocyclohexyl)-2-(3-(3-hydroxypropyl)-3,8-diazabicyclo[3.2.1]octan-8-yl)benzo[d]thiazole-6-carboxamide

Example 243 may be prepared according to Scheme 24.

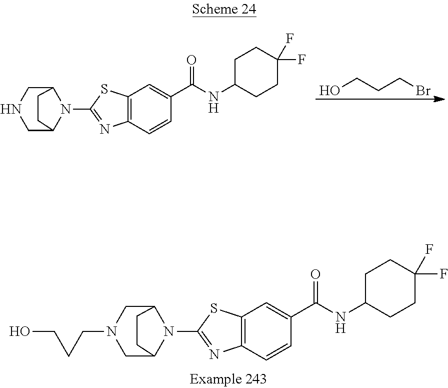 [SMILES: O=C(NC1CCC(F)(F)CC1)c1ccc2nc(N3C4CCC3CNC4)sc2c1 ; OCCCBr ; O=C(NC1CCC(F)(F)CC1)c1ccc2nc(N3C4CCC3CN(CCCO)C4)sc2c1]

To a stirred solution of 2-(3,8-diazabicyclo[3.2.1]octan-8-yl)-N-(4,4-difluorocyclohexyl)benzo[d]thiazole-6-carboxamide (100 mg, 0.25 mmol, 1 equiv.) in acetonitrile (5 mL) were added K₂CO₃ (68 mg, 0.49 mmol, 2 equiv.) and 3-bromopropanol (103 mg, 0.74 mmol, 3 equiv.) at r.t. The reaction mixture was heated at 80° C. for 16 h, then diluted with water (25 mL) and extracted with 5% methanol:DCM (3×50 mL). The combined organic extract was washed with brine (25 mL), dried over anhydrous sodium sulfate, filtered and concentrated under reduced pressure. The crude product was purified by preparative HPLC using ammonium bicarbonate as buffer to afford the title compound (70 mg, 61%) as an off-white solid. ¹H NMR δ_{H} (400 MHz, DMSO-d₆) 8.25 (1H, s), 8.10-8.23 (1H, m), 7.71-7.85 (1H, m), 7.40-7.53 (1H, m), 4.27-4.45 (3H, m), 3.90-4.10 (1H, m), 3.36-3.53 (2H, m), 2.71-2.84 (2H, m), 2.25-2.39 (4H, m), 1.81-2.14 (10H, m), 1.60-1.74 (2H, m), 1.46-1.60 (2H, m).

### Example 244: 2-(3-oxa-7,9-diazabicyclo[3.3.1]nonan-9-yl)-N-cyclopentylbenzo[d]thiazole-6-carboxamide

Example 244 may be prepared according to Scheme 25.

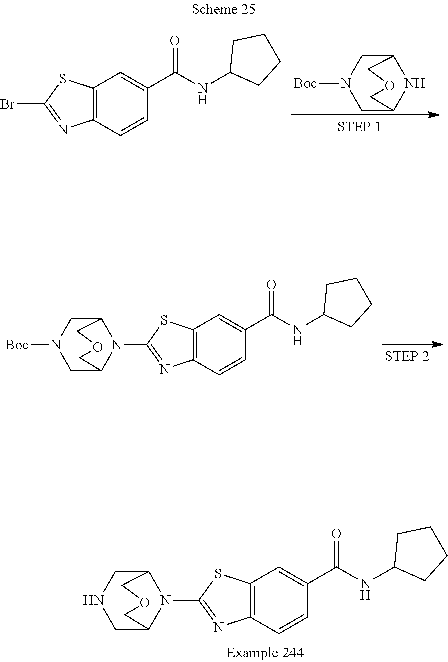

### Step 1 (Scheme 25): Buchwald-Hartwig coupling reaction

To a de-gassed solution of 2-bromo-N-cyclopentylbenzo[d]thiazole-6-carboxamide (200 mg, 0.62 mmol, 1.0 equiv.) in toluene (10 vol) under argon were added the tert-butyl 3-oxa-7,9-diazabicyclo[3.3.1]nonane-7-carboxylate (197 mg, 0.86 mmol, 1.4 equiv.), Cs₂CO₃ (400 mg, 1.23 mmol, 2.0 equiv.), (rac)-2,2′-bis(diphenylphosphino)-1, 1-binaphthyl (77 mg, 0.123 mmol, 0.2 eq) and Pd(OAc)₂ (14 mg, 0.061 mmol, 0.1 eq). The reaction mixture was stirred at 110° C. for 16 h (monitored by LCMS). The mixture was then cooled to ambient temperature, concentrated in vacuo and filtered through Celite®, washing with 10% methanol/DCM. The filtrate was concentrated to obtain a residue which was purified by preparative HPLC using ammonium bicarbonate as buffer to afford tert-butyl 9-(6-(cyclopentylcarbamoyl)benzo[d]thiazol-2-yl)-3-oxa-7,9-diazabicyclo[3.3.1]nonane-7-carboxylate (30 mg, 10%)

### Step 2 (Scheme 25): Boc deprotection to afford 2-(3-oxa-7,9-diazabicyclo[3.3.1]nonan-9-yl)-N-cyclopentylbenzo[d]thiazole-6-carboxamide

To a stirred solution of tert-butyl 9-(6-(cyclopentylcarbamoyl)benzo[d]thiazol-2-yl)-3-oxa-7,9-diazabicyclo[3.3.1]nonane-7-carboxylate (30 mg, 0.063 mmol, 1.0 eq.) in DCM (5 mL) at 0° C. was added TFA (0.025 mL, 0.32 mmol, 5 equiv.) and the reaction mixture was stirred at r.t. for 16 h. After completion of the reaction (monitored by TLC), the reaction mixture was concentrated under reduced pressure. The residue was dissolved in water, slowly basified with NaHCO₃ under cooling and then extracted with 10% methanol in DCM. The combined organic layers were washed with water and brine, dried over anhydrous sodium sulfate and concentrated under reduced pressure. The crude product was triturated with n-pentane and dried to afford 2-(3-oxa-7,9-diazabicyclo[3.3.1]nonan-9-yl)-N-cyclopentylbenzo[d]thiazole-6-carboxamide (9 mg, 37%) as an off-white solid. ¹H NMR δ_{H} (400 MHz, DMSO-d₆) 8.23-8.35 (1H, m), 8.12-8.23 (1H, m), 7.70-7.89 (1H, m), 7.37-7.53 (1H, m), 4.16-4.33 (1H, m), 4.03-4.16 (2H, m), 3.84-3.97 (4H, m), 3.15-3.26 (2H, m), 3.02-3.13 (2H, m), 1.80-1.95 (2H, m), 1.63-1.77 (2H, m), 1.48-1.60 (4H, m).

### Example 245: 2-(3,8-diazabicyclo[3.2.1]octan-3-yl)-N-(4,4-difluorocyclohexyl)-benzo[d]thiazole-6-carboxamide

Example 245 may be prepared by analogy to steps 3 to 6 of Scheme 17 but commencing with ethyl 2-bromobenzo[d]thiazole-6-carboxylate and tert-butyl 3,8-diazabicyclo[3.2.1]octane-8-carboxylate in Step 3, instead of methyl 2-bromo-4-methylbenzo[d]thiazole-6-carboxylate and tert-butyl piperazine-1-carboxylate, and using 4,4-difluorocyclohexylamine in Step 5 instead of 1-aminoindane. The title compound was isolated as the free base. ¹H NMR δ_{H} (400 MHz, DMSO-d₆) 8.24-8.28 (1H, m), 8.17-8.23 (1H, m), 7.72-7.86 (1H, m), 7.44 (1H, d, J 8.4), 3.85-4.12 (1H, m), 3.60-3.73 (2H, m), 3.46-3.60 (2H, m), 3.19-3.30 (2H, m), 1.81-2.19 (6H, m), 1.50-1.79 (6H, m).

### Example 246: N-(4,4-difluorocyclohexyl)-2-(8-methyl-3,8-diazabicyclo[3.2.1]octan-3-yl)-benzo[d]thiazole-6-carboxamide

Example 246 may be prepared by reductive amination of Example 245 with formaldehyde as follows: To a stirred solution of 2-(3,8-diazabicyclo[3.2.1]octan-3-yl)-N-(4,4-difluorocyclohexyl)benzo[d]thiazole-6-carboxamide (Example 245; 1.0 equiv.) in MeOH (20 vol.) was added formaldehyde (37% w/w in H₂O; 2.0 equiv.) and acetic acid (0.1 equiv.). The mixture was stirred at ambient temperature for 2 h and then cooled to 0° C. prior to addition of sodium cyanoborohydride (2.0 equiv.) and stirring for a further period of 16 h (monitored by TLC). The reaction mixture was quenched with ice-cold water (1.25 vol.), concentrated under reduced pressure and extracted with DCM (5 vol.). The DCM extract was washed with saturated NaHCO₃ solution (1.25 vol.) followed by brine (1.25 vol.), dried (Na₂SO₄) and evaporated to dryness under reduced pressure. The crude product was purified by preparative HPLC using ammonium bicarbonate buffer to obtain N-(4,4-difluorocyclohexyl)-2-(8-methyl-3,8-diazabicyclo[3.2.1]octan-3-yl)benzo[d]thiazole-6-carboxamide as an off-white solid. ¹H NMR δ_{H} (400 MHz, DMSO-d₆) 8.22-8.31 (1H, m), 8.14-8.23 (1H, m), 7.74-7.83 (1H, m), 7.44 (1H, d, J 8.5), 3.91-4.08 (1H, m), 3.54-3.72 (2H, m), 3.33-3.40 (2H, m), 3.16-3.27 (2H, m), 2.20-2.29 (3H, m), 1.79-2.13 (8H, m), 1.48-1.74 (4H, m).

### Example 247: N-(4,4-difluorocyclohexyl)-2-(8-(2-hydroxyethyl)-3,8-diazabicyclo[3.2.1]octan-3-yl)benzo[d]thiazole-6-carboxamide

Example 247 may be prepared by analogy to Example 120 of Scheme 8 but commencing with 2-(3,8-diazabicyclo[3.2.1]octan-3-yl)-N-(4,4-difluorocyclohexyl)benzo[d]thiazole-6-carboxamide (Example 245) instead of (S)-N-(chroman-4-yl)-2-(piperazin-1-yl)benzo[d]thiazole-6-carboxamide hydrochloride. ¹H NMR δ_{H} (400 MHz, DMSO-d₆) 8.24-8.28 (1H, m), 8.18-8.24 (1H, m), 7.74-7.87 (1H, m), 7.39-7.48 (1H, m), 4.39-4.57 (1H, m), 3.92-4.07 (1H, m), 3.58-3.73 (2H, m), 3.47-3.57 (2H, m), 3.36-3.44 (4H, m), 2.41-2.49 (2H, m), 1.81-2.15 (8H, m), 1.45-1.76 (4H, m).

### Example 248: N-(4,4-difluorocyclohexyl)-2-(8-(3-fluoropropyl)-3,8-diazabicyclo[3.2.1]octan-3-yl)benzo[d]thiazole-6-carboxamide

Example 248 may be prepared by analogy to Example 243 of Scheme 24 but commencing with 2-(3,8-diazabicyclo[3.2.1]octan-3-yl)-N-(4,4-difluorocyclohexyl)benzo[d]thiazole-6-carboxamide (Example 245) instead of 2-(3,8-diazabicyclo[3.2.1]octan-8-yl)-N-(4,4-difluorocyclohexyl)benzo[d]thiazole-6-carboxamide and reacting with 1-bromo-3-fluoropropane instead of 3-bromopropanol. ¹H NMR δ_{H} (400 MHz, DMSO-d₆) 8.24-8.30 (1H, m), 8.12-8.23 (1H, m), 7.74-7.84 (1H, m), 7.36-7.50 (1H, m), 4.45-4.71 (2H, dt, J 47.2, 6.0), 3.91-4.08 (1H, m), 3.53-3.71 (2H, m), 3.33-3.40 (4H, m), 2.40-2.48 (2H, m), 1.76-2.18 (10H, m), 1.44-1.75 (4H, m).

### Example 249: N-cyclopentyl-2-(3,8-diazabicyclo[3.2.1]octan-8-yl)-4-(1-hydroxypropan-2-yl)-benzo[d]thiazole-6-carboxamide

Example 249 may be prepared according to Scheme 26.

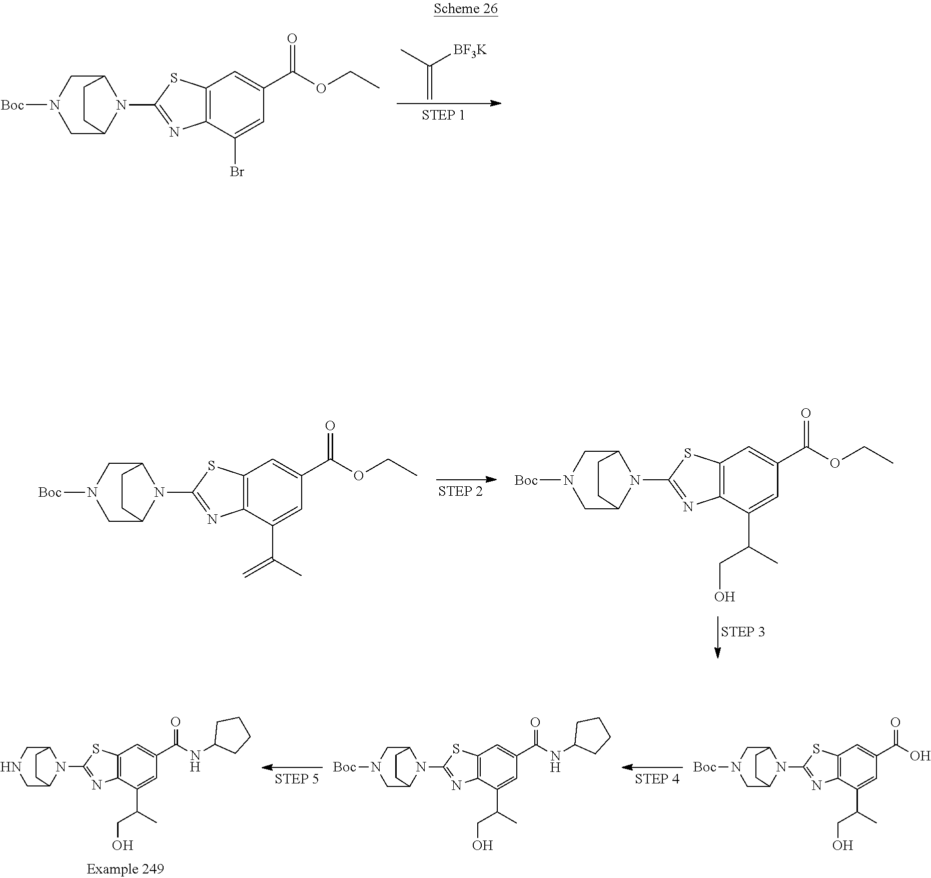

### Step 1 (Scheme 26): Synthesis of ethyl 2-(3-(tert-butoxycarbonyl)-3,8-diazabicyclo[3.2.1]octan-8-yl)-4-(prop-1-en-2-yl)benzo[d]thiazole-6-carboxylate

To a stirred solution of ethyl 4-bromo-2-(3-(tert-butoxycarbonyl)-3,8-diazabicyclo[3.2.1]octan-8-yl)benzo[d]thiazole-6-carboxylate (3.0 g, 6.04 mmol), trifluoro(prop-1-en-2-yl)-14-borane, potassium salt (1.07 g, 7.25 mmol) in 1,4-dioxane:water 10:2 (36 mL) under continuous bubbling of nitrogen was added sodium carbonate (1.92 g, 18.1 mmol) and 1,1′-bis(diphenylphosphino)ferrocene-palladium(II)dichloride dichloromethane complex (0.494 g, 0.604 mmol). The resulting mixture was stirred at 100° C. for 16 h and monitored by TLC. After completion the reaction mixture was diluted with ethyl acetate, washed with water and brine, dried over Na₂SO₄ and concentrated. The resulting crude product was purified by flash chromatography using 25-30% ethyl acetate in petroleum ether as eluent to afford ethyl 2-(3-(tert-butoxycarbonyl)-3,8-diazabicyclo[3.2.1]octan-8-yl)-4-(prop-1-en-2-yl)benzo[d]thiazole-6-carboxylate (2.10 g, 74%) as an off-white solid.

### Step 2 (Scheme 26): Synthesis of ethyl 2-(3-(tert-butoxycarbonyl)-3,8-diazabicyclo[3.2.1]octan-8-yl)-4-(1-hydroxypropan-2-yl)benzo[d]thiazole-6-carboxylate

To a stirred solution of ethyl 2-(3-(tert-butoxycarbonyl)-3,8-diazabicyclo[3.2.1]octan-8-yl)-4-(prop-1-en-2-yl)benzo[d]thiazole-6-carboxylate (1.30 g, 2.84 mmol), in THF (50 mL) at 0° C., was added borane tetrahydrofuran complex (1.0 M; 8.52 mL, 8.52 mmol) and the mixture stirred at r.t. for 16 h. To the reaction mixture was added aqueous sodium hydroxide (1.0 M; 2.84 mL, 2.84 mmol) and 30% hydrogen peroxide (0.322 g, 2.84 mmol) and the mixture stirred at r.t. for 1 h. The reaction mixture was extracted with ethyl acetate (2×100 mL), the combined organic layers were washed with water and brine, dried over Na₂SO₄, and concentrated under reduced pressure. The resulting crude product was purified by flash chromatography using 25% EtOAc in hexane as eluent to afford ethyl 2-(3-(tert-butoxycarbonyl)-3,8-diazabicyclo[3.2.1]octan-8-yl)-4-(1-hydroxypropan-2-yl)benzo[d]thiazole-6-carboxylate (350 mg, 18%) as an off-white solid.

### Step 3 (Scheme 26): Synthesis of 2-(3-(tert-butoxycarbonyl)-3,8-diazabicyclo[3.2.1]octan-8-yl)-4-(1-hydroxypropan-2-yl)benzo[d]thiazole-6-carboxylic acid

To a stirred solution of ethyl 2-(3-(tert-butoxycarbonyl)-3,8-diazabicyclo[3.2.1]octan-8-yl)-4-(1-hydroxypropan-2-yl)benzo[d]thiazole-6-carboxylate (320 mg, 0.67 mmol) in THF: methanol: water (2:2:1) was added lithium hydroxide monohydrate (55.2 mg, 1.35 mmol). The mixture was stirred at r.t. for 3 h, monitoring by LCMS. The reaction mixture was concentrated under reduced pressure. The residue was diluted with water, acidified with citric acid solution (to pH 4 to 5) and extracted with DCM. The combined organic layers were washed with water and brine, dried over Na₂SO₄ and concentrated to afford 2-(3-(tert-butoxycarbonyl)-3,8-diazabicyclo[3.2.1]octan-8-yl)-4-(1-hydroxypropan-2-yl)benzo[d]thiazole-6-carboxylic acid (210 mg, 58%) as an off-white solid.

### Step 4 (Scheme 26): Synthesis of N-cyclopentyl-2-(3-(tert-butoxycarbonyl)-3,8-diazabicyclo[3.2.1]octan-8-yl)-4-(1-hydroxypropan-2-yl)benzo[d]thiazole-6-carboxamide

To a stirred solution of 2-(3-(tert-butoxycarbonyl)-3,8-diazabicyclo[3.2.1]octan-8-yl)-4-(1-hydroxypropan-2-yl)benzo[d]thiazole-6-carboxylic acid (90 mg, 0.20 mmol) in DCM (5 mL), were added cyclopentanamine (18.8 mg, 0.22 mmol) and n-propylphosphonic acid anhydride, cyclic trimer (50% in EtOAc) (128 mg, 0.40 mmol). N,N-diisopropylethylamine (130 mg, 1.0 mmol) was added and the mixture stirred at r.t. for 16 h, monitoring by TLC. The reaction mixture was diluted with ice-cold water and extracted with DCM (2×25 mL). The combined organic layers were washed with water and brine, dried over Na₂SO₄ and concentrated under reduced pressure to afford N-cyclopentyl-2-(3-(tert-butoxycarbonyl)-3,8-diazabicyclo[3.2.1]octan-8-yl)-4-(1-hydroxypropan-2-yl)benzo[d]thiazole-6-carboxamide (95 mg, 72%) as an off-white solid.

### Step 5 (Scheme 26): Synthesis of 2-(3,8-diazabicyclo[3.2.1]octan-8-yl)-N-cyclopentyl-4-(1-hydroxypropan-2-yl)benzo[d]thiazole-6-carboxamide

To a stirred solution of N-cyclopentyl-2-(3-(tert-butoxycarbonyl)-3,8-diazabicyclo[3.2.1]octan-8-yl)-4-(1-hydroxypropan-2-yl)benzo[d]thiazole-6-carboxamide (80 mg, 0.16 mmol) in DCM (5 mL), was added HCl (4M in 1,4-dioxane; 0.039 mL, 0.16 mmol). The mixture was stirred at r.t. for 3 h, then concentrated under reduced pressure. The residue was purified by preparative HPLC using ammonium bicarbonate as buffer to afford 2-(3,8-diazabicyclo[3.2.1]octan-8-yl)-N-cyclopentyl-4-(1-hydroxypropan-2-yl)benzo[d]thiazole-6-carboxamide (21 mg, 32%) as an off white solid. ¹H NMR δ_{H} (400 MHz, DMSO-d₆) 8.12-8.19 (1H, m), 8.06-8.11 (1H, m), 7.61-7.68 (1H, m), 4.58-4.67 (1H, m), 4.18-4.29 (3H, m), 3.67-3.75 (1H, m), 3.40-3.59 (2H, m), 2.94-3.04 (2H, m), 2.58-2.71 (2H, m), 1.95-2.03 (4H, m), 1.83-1.94 (2H, m), 1.64-1.77 (2H, m), 1.47-1.62 (4H, m), 1.26-1.32 (3H, m).

### Example 250: 2-(3,8-diazabicyclo[3.2.1]octan-8-yl)-N-cyclopentyl-4-(2-hydroxyethyl)benzo[d]thiazole-6-carboxamide

Example 250 may be prepared by analogy to Example 249, Scheme 26, using trifluoro(vinyl)-14-borane, potassium salt instead of trifluoro(prop-1-en-2-yl)-14-borane, potassium salt in Step 1. ¹H NMR δ_{H} (400 MHz, DMSO-d₆) 8.11-8.15 (1H, m), 8.08 (1H, d, J 1.7), 7.65 (1H, d, J 1.7), 4.68 (1H, t, J 5.3), 4.22 (3H, br s), 3.68-3.74 (2H, m), 2.96-3.06 (3H, m), 2.90-3.09 (1H, m), 2.60-2.68 (2H, m), 1.94-2.01 (4H, m), 1.82-1.94 (2H, m), 1.64-1.77 (2H, m), 1.46-1.63 (4H, m).

### Examples 251 to 259

Examples 251 to 259 may be prepared according to Scheme 27.

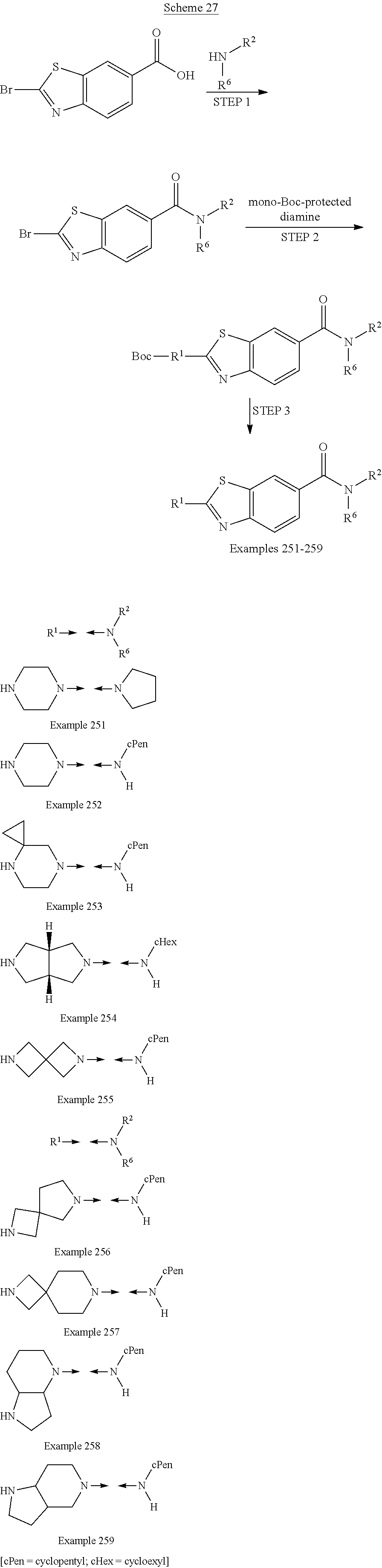

### Step 1 (Scheme 27): General Procedure for Amide Coupling

To a solution of 2-bromobenzo[d]thiazole-6-carboxylic acid (1.0 eq.) in DCM (50 mL) at 0° C. were added n-propylphosphonic acid anhydride, cyclic trimer (50% in EtOAc) (2.0 equiv.), DIPEA (4.0 equiv.) and the required amine (2.0 equiv.) and the mixture stirred at r.t. for 16 h, monitoring by TLC. The reaction mixture was concentrated, diluted with ice-cold water and extracted with ethyl acetate (3×). The combined organic extract was washed with brine, dried over anhydrous sodium sulfate, filtered and concentrated under reduced pressure. The residue was purified by flash column chromatography using 50% to 100% ethyl acetate in petroleum ether as eluent to obtain the required 2-bromobenzo[d]thiazole-6-carboxamide derivatives as off-white solids.

### Step 2 (Scheme 27): General Procedure for SNAr Reaction

In a two-necked round bottomed flaskwas taken the 2-bromobenzo[d]thiazole-6-carboxamide derivative from Step 1 (1 equiv.) in acetonitrile (10 vol.) and to this were added K₂CO₃ (3 equiv.) and the required Boc protected diamine (2 equiv.). The reaction mixture was stirred at 80° C. for 16 h. The completion of the reaction was monitored by UPLC. The reaction mixture was concentrated under reduced pressure, diluted with water and extracted with 10% methanol in DCM (3 times). The combined organic extracts were washed with brine, dried over anhydrous sodium sulfate, filtered and concentrated under reduced pressure. The residue was purified by flash column chromatography using 0% to 5% methanol in DCM as eluent to obtain the required Boc protected 2-aminobenzo[d]thiazole-6-carboxamide derivatives as off-white solids.

### Step 3 (Scheme 27) leading to Examples 251 to 259: General procedure for Boc-deprotection

To a stirred solution of Boc protected 2-aminobenzo[d]thiazole-6-carboxamide derivative from Step 2 (1 equiv.) in DCM (0.2 M in substrate) at 0° C. was added 4 M HCl in 1,4-dioxane (8.0 equiv. HCl). The reaction mixture was stirred at ambient temperature for 16 h (monitored by TLC). The mixture was concentrated under reduced pressure and the resulting residue was purified by preparative HPLC using ammonium bicarbonate as buffer to obtain Examples 251 to 259 as off-white solids.

### Example 251: (2-(piperazin-1-yl)benzo[d]thiazol-6-yl)(pyrrolidin-1-yl)methanone

Prepared according to Scheme 27, using pyrrolidine as the amine in Step 1 and N-Boc piperazine as the Boc protected diamine in Step 2. ¹H NMR δ_{H} (400 MHz, DMSO-d₆) 7.91-8.03 (1H, m), 7.37-7.52 (2H, m), 3.40-3.60 (8H, m), 2.72-2.88 (4H, m), 1.74-1.96 (4H, m).

### Example 252: 2-(3,6-diazabicyclo[3.1.1]heptan-6-yl)-N-cyclopentylbenzo[d]thiazole-6-carboxamide

Prepared according to Scheme 27, using cyclopentylamine as the amine in Step 1 and 3-(tert-butoxycarbonyl)-3,6-diazabicyclo[3.1.1]heptane as the Boc protected diamine in Step 2. ¹H NMR δ_{H} (400 MHz, DMSO-d₆) 8.24-8.33 (1H, m), 8.14-8.23 (1H, m), 7.76-7.85 (1H, m), 7.44-7.55 (1H, m), 4.32-4.44 (2H, m), 4.16-4.30 (1H, m), 3.43-3.55 (2H, m), 2.81-2.97 (2H, m), 2.70-2.80 (1H, m), 1.87-1.97 (3H, m), 1.65-1.76 (2H, m), 1.49-1.60 (4H, m).

### Example 253: N-cyclopentyl-2-(4,7-diazaspiro[2.5]octan-7-yl)benzo[d]thiazole-6-carboxamide

Prepared according to Scheme 27, using cyclopentylamine as the amine in Step 1 and 4-(tert-butoxycarbonyl)-4,7-diazaspiro[2.5]octane as the Boc protected diamine in Step 2. ¹H NMR δ_{H} (400 MHz, DMSO-d₆) 8.20-8.26 (1H, m), 8.12-8.22 (1H, m), 7.74-7.82 (1H, m), 7.36-7.46 (1H, m), 4.16-4.30 (1H, m), 3.53-3.61 (2H, m), 3.42-3.50 (2H, m), 2.82-2.94 (2H, m), 2.56-2.65 (1H, m), 1.81-1.96 (2H, m), 1.62-1.78 (2H, m), 1.47-1.61 (4H, m), 0.54-0.62 (2H, m), 0.44-0.54 (2H, m).

### Example 254: N-cyclohexyl-2-((3aR,6aS)-hexahydropyrrolo[3,4-c]pyrrol-2 (1H)-yl)benzo[d]thiazole-6-carboxamide

Prepared according to Scheme 27, using cyclohexylamine as the amine in Step 1 and tert-butyl (3aR,6aS)-hexahydropyrrolo[3,4-c]pyrrole-2 (1H)-carboxylate as the Boc protected diamine in Step 2. ¹H NMR δ_{H} (400 MHz, DMSO-d₆) 8.21-8.32 (1H, m), 8.02-8.15 (1H, m), 7.68-7.83 (1H, m), 7.45 (1H, d, J 8.4), 3.67-3.88 (3H, m), 3.32-3.36 (2H, m), 2.83-2.98 (4H, m), 2.62-2.76 (2H, m), 1.68-1.94 (4H, m), 1.56-1.67 (1H, m), 1.22-1.42 (4H, m), 1.03-1.22 (1H, m).

### Example 255: N-cyclopentyl-2-(2,6-diazaspiro[3.3]heptan-2-yl)benzo[d]thiazole-6-carboxamide

Prepared according to Scheme 27, using cyclopentylamine as the amine in Step 1 and tert-butyl 2,6-diazaspiro[3.3]heptane-2-carboxylate as the Boc protected diamine in Step 2. ¹H NMR δ_{H} (400 MHz, DMSO-d₆) 8.22-8.31 (1H, m), 8.11-8.22 (1H, m), 7.70-7.84 (1H, m), 7.37-7.53 (1H, m), 4.16-4.32 (5H, m), 3.59-3.70 (4H, m), 1.81-1.92 (2H, m), 1.62-1.78 (2H, m), 1.47-1.59 (4H, m).

### Example 256: N-cyclopentyl-2-(2,6-diazaspiro[3.4]octan-6-yl)benzo[d]thiazole-6-carboxamide

Prepared according to Scheme 27, using cyclopentylamine as the amine in Step 1 and tert-butyl 2,6-diazaspiro[3.4]octane-2-carboxylate as the Boc protected diamine in Step 2. ¹H NMR δ_{H} (400 MHz, DMSO-d₆) 8.20-8.30 (1H, m), 8.10-8.20 (1H, m), 7.78 (1H, dd, J 8.4, 1.8), 7.45 (1H, d, J 8.4), 4.13-4.36 (1H, m), 3.61-3.69 (2H, m), 3.47-3.58 (4H, m), 3.36-3.46 (2H, m), 2.22 (2H, t, J 6.9), 1.80-1.94 (2H, m), 1.70 (2H, m), 1.47-1.62 (4H, m).

### Example 257: N-cyclopentyl-2-(2,7-diazaspiro[3.5]nonan-7-yl)benzo[d]thiazole-6-carboxamide

Prepared according to Scheme 27, using cyclopentylamine as the amine in Step 1 and tert-butyl 2,7-diazaspiro[3.5]nonane-2-carboxylate as the Boc protected diamine in Step 2. ¹H NMR δ_{H} (400 MHz, DMSO-d₆) 8.22-8.29 (1H, m), 8.12-8.22 (1H, m), 7.74-7.83 (1H, m), 7.36-7.48 (1H, m), 4.14-4.30 (1H, m), 3.62 (1H, br s), 3.54-3.60 (4H, m), 3.47-3.54 (3H, m), 1.79-1.96 (6H, m), 1.61-1.77 (2H, m), 1.50-1.59 (4H, m).

### Example 258: N-cyclopentyl-2-(octahydro-4H-pyrrolo[3,2-b]pyridin-4-yl)benzo[d]thiazole-6-carboxamide

Prepared according to Scheme 27, using cyclopentylamine as the amine in Step 1 and tert-butyl octahydro-1H-pyrrolo[3,2-b]pyridine-1-carboxylate as the Boc protected diamine in Step 2. ¹H NMR δ_{H} (400 MHz, DMSO-d₆) 8.22-8.30 (1H, m), 8.11-8.22 (1H, m), 7.73-7.84 (1H, m), 7.38-7.50 (1H, m), 4.36-4.50 (1H, m), 4.16-4.31 (1H, m), 3.80-3.92 (1H, m), 3.12-3.24 (2H, m), 2.88-3.08 (2H, m), 2.01-2.21 (1H, m), 1.82-1.95 (2H, m), 1.61-1.82 (5H, m), 1.35-1.62 (6H, m).

### Example 259: N-cyclopentyl-2-(octahydro-5H-pyrrolo[3,2-c]pyridin-5-yl)benzo[d]thiazole-6-carboxamide

Prepared according to Scheme 27, using cyclopentylamine as the amine in Step 1 and tert-butyl octahydro-1H-pyrrolo[3,2-c]pyridine-1-carboxylate as the Boc protected diamine in Step 2. ¹H NMR δ_{H} (400 MHz, DMSO-d₆) 8.19-8.30 (1H, m), 8.07-8.20 (1H, m), 7.69-7.86 (1H, m), 7.41 (1H, d, J 8.5), 4.07-4.32 (1H, m), 3.68-3.79 (1H, m), 3.44-3.68 (2H, m), 3.11-3.24 (2H, m), 2.86-2.98 (1H, m), 2.70-2.81 (1H, m), 2.21-2.30 (1H, m), 1.83-1.93 (4H, m), 1.63-1.79 (3H, m), 1.49-1.61 (4H, m), 1.30-1.46 (1H, m).

### Examples 260 to 266

Examples 260 to 266 may be prepared by reductive alkylation of the secondary amines of Examples 253 to 259 as follows: General procedure for reductive alkylation of amines

To a stirred solution of the amine (1.0 equiv.) in MeOH (10 vol) was added formaldehyde (37% w/w in H₂O; 2.0 equiv.) and acetic acid (0.1 equiv.). The mixture was stirred at ambient temperature for 6 h and then cooled to 0° C. prior to addition of sodium cyanoborohydride (2.0 equiv.), stirring for a further period of 16 h (monitored by TLC). The reaction mixture was quenched with ice-cold water and concentrated under reduced pressure. The aqueous layer was diluted with water and extracted with DCM. The combined organic extracts were washed with saturated aq. NaHCO₃ followed by brine, dried over Na₂SO₄ and concentrated under reduced pressure. The residue was purified by preparative HPLC using ammonium bicarbonate buffer to obtain the title compounds as off-white solids.

### Example 260: N-cyclopentyl-2-(4-methyl-4,7-diazaspiro[2.5]octan-7-yl)benzo[d]thiazole-6-carboxamide

Prepared by reductive alkylation of Example 253 with formaldehyde. ¹H NMR δ_{H} (400 MHz, DMSO-d₆) 8.22-8.27 (1H, m), 8.14-8.22 (1H, m), 7.75-7.82 (1H, m), 7.39-7.47 (1H, m), 4.17-4.28 (1H, m), 3.58-3.66 (2H, m), 3.46-3.52 (2H, m), 2.91-2.97 (2H, m), 2.32-2.36 (3H, m), 1.82-1.95 (2H, m), 1.64-1.76 (2H, m), 1.49-1.61 (4H, m), 0.61-0.67 (2H, m), 0.54-0.60 (2H, m).

### Example 261: N-cyclohexyl-2-((3aR,6aS)-5-methylhexahydropyrrolo[3,4-c]pyrrol-2 (1H)-yl)benzo[d]thiazole-6-carboxamide

Prepared by reductive alkylation of Example 254 with formaldehyde. ¹H NMR δ_{H} (400 MHz, DMSO-d₆) 8.18-8.33 (1H, m), 7.94-8.16 (1H, m), 7.71-7.86 (1H, m), 7.37-7.54 (1H, m), 3.65-3.88 (3H, m), 3.36-3.47 (2H, m), 2.91-3.07 (2H, m), 2.56-2.47 (4H, m), 2.23 (3H, s), 1.68-1.89 (4H, m), 1.52-1.67 (1H, m), 1.24-1.38 (4H, m), 1.06-1.22 (1H, m).

### Example 262: N-cyclopentyl-2-(6-methyl-2,6-diazaspiro[3.3]heptan-2-yl)benzo[d]thiazole-6-carboxamide

Prepared by reductive alkylation of Example 255 with formaldehyde. ¹H NMR δ_{H} (400 MHz, DMSO-d₆) 8.23-8.27 (1H, m), 8.14-8.20 (1H, m), 7.75-7.81 (1H, m), 7.44-7.50 (1H, m), 4.19-4.25 (5H, m), 3.28-3.34 (4H, m), 2.17 (3H, s), 1.80-1.95 (2H, m), 1.65-1.75 (2H, m), 1.47-1.59 (4H, m).

### Example 263: N-cyclopentyl-2-(2-methyl-2,6-diazaspiro[3.4]octan-6-yl)benzo[d]thiazole-6-carboxamide

Prepared by reductive alkylation of Example 256 with formaldehyde. ¹H NMR δ_{H} (400 MHz, DMSO-d₆) 8.23-8.26 (1H, m), 8.12-8.17 (1H, m), 7.76-7.81 (1H, m), 7.43-7.47 (1H, m), 4.18-4.27 (1H, m), 3.60-3.64 (2H, m), 3.50-3.57 (2H, m), 3.11-3.18 (4H, m), 2.24 (3H, s) 2.16-2.23 (2H, m), 1.82-1.94 (2H, m), 1.64-1.76 (2H, m) 1.48-1.61 (4H, m).

### Example 264: N-cyclopentyl-2-(2-methyl-2,7-diazaspiro[3.5]nonan-7-yl)benzo[d]thiazole-6-carboxamide

Prepared by reductive alkylation of Example 257 with formaldehyde. ¹H NMR δ_{H} (400 MHz, DMSO-d₆) 8.21-8.27 (1H, m), 8.09-8.21 (1H, m), 7.69-7.85 (1H, m), 7.42 (1H, d, J 8.5), 4.12-4.30 (1H, m), 3.50-3.61 (4H, m), 2.95-3.01 (4H, m), 2.25 (3H, s), 1.82-1.96 (2H, m), 1.73-1.82 (4H, m), 1.67-1.73 (2H, m), 1.48-1.58 (4H, m).

### Example 265: N-cyclopentyl-2-(1-methyloctahydro-4H-pyrrolo[3,2-b]pyridin-4-yl)benzo[d]thiazole-6-carboxamide

Prepared by reductive alkylation of Example 258 with formaldehyde. ¹H NMR δ_{H} (400 MHz, DMSO-d₆) 8.22-8.29 (1H, m), 8.10-8.20 (1H, m), 7.68-7.86 (1H, m), 7.39-7.48 (1H, m), 4.51-4.66 (1H, m), 4.16-4.30 (1H, m), 3.67-3.80 (1H, m), 3.35-3.48 (1H, m), 2.83-2.97 (1H, m), 2.22-2.43 (6H, m), 1.80-1.96 (3H, m), 1.43-1.80 (10H, m).

### Example 266: N-cyclopentyl-2-(1-methyloctahydro-1H-pyrrolo[3,2-c]pyridin-5-yl)benzo[d]thiazole-6-carboxamide

Prepared by reductive alkylation of Example 259 with formaldehyde. ¹H NMR δ_{H} (400 MHz, DMSO-d₆) 8.18-8.27 (1H, m), 8.09-8.20 (1H, m), 7.72-7.84 (1H, m), 7.36-7.47 (1H, m), 4.15-4.33 (1H, m), 3.65-3.81 (1H, m), 3.42-3.60 (2H, m), 3.32-3.39 (1H, m), 2.91-3.04 (1H, m), 2.28-2.46 (2H, m), 2.19-2.27 (3H, m), 2.02-2.19 (1H, m), 1.76-1.99 (5H, m), 1.62-1.75 (2H, m), 1.45-1.61 (4H, m), 1.24-1.42 (1H, m).

### Example 267: (S)-N-(chroman-4-yl)-N-methyl-2-(piperazin-1-yl)benzo[d]thiazole-6-carboxamide

Example 267 may be prepared according to Scheme 28

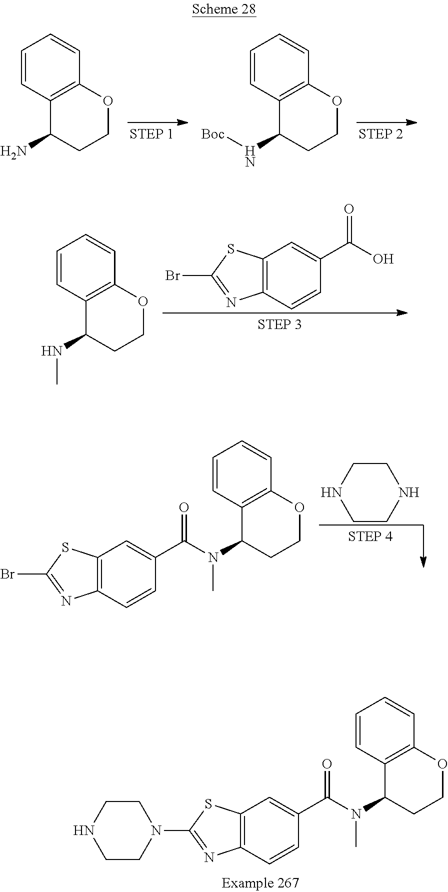

### Step 1 (Scheme 28): Synthesis of tert-butyl (S)-chroman-4-ylcarbamate

To a stirred solution of (S)-chroman-4-amine, HCl salt (500 mg, 2.69 mmol) in THF (10 mL) at 0° C. were added DIPEA (1.88 mL, 10.8 mmol) and Boc-anhydride (0.75 mL, 3.23 mmol). The reaction mixture was stirred r.t. for 5 h, monitoring by LCMS, then concentrated under reduced pressure. The residue was diluted with water (10 mL) and extracted with 10% MeOH in DCM (2×10 mL). The combined organic extracts were washed with brine (10 mL), dried over anhydrous sodium sulfate, filtered and concentrated under reduced pressure. The crude product was purified by flash column chromatography, using 0 to 40% ethyl acetate in petroleum ether as eluent, to obtain tert-butyl (S)-chroman-4-ylcarbamate (600 mg, 2.4 mmol, 89% yield) as an off-white solid.

### Step 2 (Scheme 28): Synthesis of (S)-N-methylchroman-4-amine

To a stirred solution of tert-butyl (S)-chroman-4-ylcarbamate (280 mg, 1.12 mmol) in THF (10 mL) at 0° C. was added LiAlH₄ (1.0 M in THF; 2.25 mL, 2.25 mmol). The mixture was allowed to warm to room temperature and maintained at 25° C. for 1 h followed by stirring at 60° C. for 16 h. The reaction was monitored by TLC. The reaction was quenched with saturated aq. sodium sulfate solution and concentrated under reduced pressure. The residue was diluted with water (10 mL) and the organic components were extracted with 10% MeOH in DCM (2×10 mL). The combined organic extracts were washed with brine (10 mL), dried over anhydrous sodium sulfate, filtered and concentrated under reduced pressure to afford crude (S)-N-methylchroman-4-amine (170 mg, 1.022 mmol, 91% yield), which was used directly in the next step.

### Step 3 (Scheme 28): Synthesis of (S)-2-bromo-N-(chroman-4-yl)-N-methylbenzo[d]thiazole-6-carboxamide

To a stirred solution of 2-bromobenzo[d]thiazole-6-carboxylic acid (190 mg, 0.74 mmol) in DCM (10 mL) at 0° C. was added DIPEA (0.50 mL, 2.7 mmol) and n-propylphosphonic acid anhydride, cyclic trimer (50% in EtOAc; 0.81 mL, 1.35 mmol). The mixture was stirred for 15 min, after which was added crude (S)-N-methylchroman-4-amine (110 mg, 0.674 mmol). The mixture was stirred at r.t. for 16 h, then diluted with water (10 mL) and extracted with 10% MeOH in DCM (2×10 mL). The combined organic extracts were washed with brine (10 mL), dried over anhydrous sodium sulfate, filtered and concentrated under reduced pressure. The residue was purified by flash column chromatography using 0 to 20% methanol in DCM as eluent to obtain (S)-2-bromo-N-(chroman-4-yl)-N-methylbenzo[d]thiazole-6-carboxamide (250 mg, 0.53 mmol, 78% yield) as an off-white solid.

### Step 4 (Scheme 28): Synthesis of (S)-N-(chroman-4-yl)-N-methyl-2-(piperazin-1-yl)benzo[d]thiazole-6-carboxamide

To a stirred solution of (S)-2-bromo-N-(chroman-4-yl)-N-methylbenzo[d]thiazole-6-carboxamide (250 mg, 0.62 mmol) in acetonitrile (10 mL) was added K₂CO₃ (257 mg, 1.86 mmol) and piperazine (64.1 mg, 0.74 mmol). The mixture was stirred at 80° C. for 16 h, then diluted with water (10 mL) and extracted with 10% MeOH in DCM (2×10 mL). The combined organic extracts were washed with brine (10 mL), dried over anhydrous sodium sulfate, filtered and concentrated under reduced pressure. The residue was purified by preparative HPLC using ammonium bicarbonate as buffer to obtain (S)-N-(chroman-4-yl)-N-methyl-2-(piperazin-1-yl)benzo[d]thiazole-6-carboxamide (12 mg, 0.03 mmol, 4.7% yield) as an off-white solid.

A mixture of two distinct amide rotamers was seen in the NMR spectrum at ambient temperature in DMSO-d₆. These merged upon heating at 80° C. ¹H NMR δ_{H} (80° C.; 400 MHz, DMSO-d₆) 7.93 (1H, d, J 1.6), 7.47 (1H, d, J8.4), 7.42 (1H, dd, J 1.6, 8.4), 7.18-7.14 (2H, m), 6.97-6.93 (1H, m), 6.80 (1H, d, J 8.0), 5.55 (1H, br s), 4.35-4.30 (1H, m), 4.16-4.11 (1H, m), 3.55-3.53 (4H, m), 2.87-2.84 (4H, m), 2.68 (3H, s), 2.33-2.10 (2H, m).

### Example 268: (S)-N-(chroman-4-yl)-N-methyl-2-(2-methylpyridin-3-yl)benzo[d]thiazole-6-carboxamide

To a solution of 2-(2-methylpyridin-3-yl)benzo[d]thiazole-6-carboxylic acid (prepared as described in Scheme 1, Step 3; 70 mg, 0.25 mmol, 1.0 equiv.) in DCM (20 mL) at 0° C. was added n-propylphosphonic acid anhydride cyclic trimer (50% in EtOAc; 165 mg, 2.0 equiv.) and DIPEA (0.14 ml, 4.0 equiv.). The mixture was stirred at 0° C. for 15 min prior to addition of the crude (S)-N-methylchroman-4-amine (prepared as described in Scheme 28, Step 2; 51 mg, 1.2 equiv.). The reaction mixture was stirred at r.t. for 16 h, then diluted with DCM and washed with water followed by brine. The organic phase was dried (Na₂SO₄), filtered and concentrated under reduced pressure. The residue was purified by preparative HPLC to afford the title compound (21 mg, 20% yield) as an off-white solid.

A 1:1 mixture of two distinct amide rotamers was seen in the NMR; split resonances are listed as “0.5H”. ¹H NMR δ_{H} (400 MHz, DMSO-d₆) 8.61-8.72 (1H, m), 8.37-8.52 (1H, m), 8.12-8.31 (2H, m), 7.66-7.81 (1H, m), 7.43-7.52 (1H, m), 7.12-7.28 (2H, m), 6.91-7.02 (1H, m), 6.73-6.90 (1H, m), 5.91-6.05 (0.5H, m) 4.99-5.11 (0.5H, m) 4.34-4.48 (0.5H, m), 4.23-4.34 (1H, m), 3.95-4.10 (0.5H, m), 2.79-2.93 (3H, m), 2.59-2.78 (3H, m), 2.06-2.40 (2H, m).

### Example 269: (2-(piperidin-4-yl)benzo[d]thiazol-6-yl)(pyrrolidin-1-yl)methanone

Prepared according to Scheme 9, using pyrrolidine as the amine component. ¹H NMR (free base form) δ_{H} (400 MHz, DMSO-d₆) 8.20-8.32 (1H, m), 7.90-8.03 (1H, m), 7.54-7.68 (1H, m), 3.39-3.59 (4H, m), 3.14-3.30 (1H, m), 2.94-3.12 (2H, m), 2.57-2.71 (2H, m), 1.97-2.11 (2H, m), 1.77-1.96 (4H, m), 1.59-1.75 (2H, m).

### Example 270: morpholino(2-(piperidin-4-yl)benzo[d]thiazol-6-yl)methanone

Prepared according to Scheme 9, using morpholine as the amine component. ¹H NMR (free base form) δ_{H} (400 MHz, DMSO-d₆) 8.09-8.19 (1H, m), 7.90-8.03 (1H, m), 7.42-7.57 (1H, m), 4.06-4.21 (1H, m), 3.32-3.50 (5H, m), 3.14-3.30 (2H, m), 2.93-3.06 (2H, m), 2.53-2.64 (4H, m), 1.88-2.10 (2H, m), 1.48-1.77 (2H, m).

### Example 271: N-cyclopentyl-N-methyl-2-(piperidin-4-yl)benzo[d]thiazole-6-carboxamide

Prepared according to Scheme 9, using N-cyclopentyl-N-methylamine as the amine component. ¹H NMR (free base form) δ_{H} (400 MHz, DMSO-d₆) 8.05-8.19 (1H, m), 7.93-8.03 (1H, m), 7.42-7.51 (1H, m), 3.98-4.01 (1H, m), 3.17-3.28 (1H, m), 2.94-3.13 (2H, m), 2.78-2.91 (3H, m), 2.58-2.67 (2H, m), 1.96-2.11 (2H, m), 1.57-1.83 (9H, m), 1.32-1.58 (2H, m).

### Example 272: 2-(piperidin-4-yl)-N-(pyridin-4-ylmethyl)benzo[d]thiazole-6-carboxamide

Prepared according to Scheme 9, using (pyridin-4-yl)methylamine as the amine component.

¹H NMR (free base form) δ_{H} (400 MHz, DMSO-d₆) 9.22-9.37 (1H, m), 8.61-8.70 (1H, m), 8.46-8.57 (2H, m), 7.98-8.10 (2H, m), 7.30-7.39 (2H, m), 4.49-4.61 (2H, m), 3.25-3.46 (1H, m), 3.05-3.24 (2H, m), 2.71-2.93 (2H, m), 2.10-2.18 (2H, m), 1.69-1.93 (2H, m).

### Example 273: 2-(piperidin-4-yl)-N-(pyridin-3-ylmethyl)benzo[d]thiazole-6-carboxamide

Prepared according to Scheme 9, using (pyridin-3-yl)methylamine as the amine component.

¹H NMR (free base form) δ_{H} (400 MHz, DMSO-d₆) 9.16-9.29 (1H, m), 8.57-8.63 (2H, m), 8.42-8.52 (1H, m), 7.93-8.13 (2H, m), 7.69-7.82 (1H, m), 7.31-7.43 (1H, m), 4.53 (2H, d, J 5.9), 3.33-3.43 (1H, m), 3.14-3.27 (2H, m), 2.78-2.94 (2H, m), 2.08-2.24 (2H, m), 1.71-1.95 (2H, m).

### Example 274: N-[(1R,2R)-2-hydroxycyclopentyl]-2-(1-methylpiperidin-4-yl)benzo[d]thiazole-6-carboxamide

Prepared according to Scheme 10, using (1R,2R)-2-aminocyclopentan-1-ol as the amine component. ¹H NMR (free base form) δ_{H} (400 MHz, DMSO-d₆) 8.52-8.63 (1H, m), 8.28-8.40 (1H, m), 7.88-8.06 (2H, m), 4.71-4.89 (1H, m), 3.96-4.11 (2H, m), 3.07-3.22 (1H, m), 2.85-3.01 (2H, m), 2.28 (3H, s), 2.08-2.22 (4H, m), 1.94-2.08 (1H, m), 1.79-1.93 (3H, m) 1.59-1.76 (2H, m), 1.42-1.57 (2H, m).

### Example 275: 2-(1-ethylpiperidin-4-yl)-N-(pyridin-4-ylmethyl)benzo[d]thiazole-6-carboxamide

Prepared according to Scheme 11, using (pyridin-4-yl)methylamine as the amine component.

¹H NMR (free base form) δ_{H} (400 MHz, DMSO-d₆) 9.17-9.25 (1H, m), 8.55-8.63 (2H, m), 8.44-8.50 (1H, m), 7.93-8.09 (2H, m), 7.73-7.78 (1H, m), 7.34-7.39 (1H, m), 4.53 (2H, d, J5.8), 3.06-3.21 (1H, m), 2.96 (2H, br d, J 11.6), 2.37 (2H, q, J7.2), 1.96-2.19 (4H, m), 1.74-1.86 (2H, m), 1.02 (3H, t, J 7.2).

### Example 276: 2-(1-ethylpiperidin-4-yl)-N-(pyridin-3-ylmethyl)benzo[d]thiazole-6-carboxamide

Prepared according to Scheme 11, using (pyridin-3-yl)methylamine as the amine component.

¹H NMR (free base form) δ_{H} (400 MHz, DMSO-d₆) 9.25 (1H, t, J5.9), 8.61-8.66 (1H, m), 8.48-8.56 (2H, m), 7.98-8.07 (2H, m), 7.32-7.37 (2H, m), 4.54 (2H, d, J 5.9), 3.11-3.21 (1H, m), 2.96 (2H, br d, J 11.3), 2.37 (2H, q, J 7.2), 1.96-2.20 (4H, m), 1.74-1.89 (2H, m), 1.03 (3H, t, J 7.2).

### Example 277: 2-(1-ethylpiperidin-4-yl)-N-(pyridazin-4-ylmethyl)benzo[d]thiazole-6-carboxamide

Prepared according to Scheme 11, using (pyridazin-4-yl)methylamine as the amine component. ¹H NMR (free base form) δ_{H} (400 MHz, DMSO-d₆) 9.27-9.33 (1H, m), 9.23-9.26 (1H, m), 9.15-9.19 (1H, m), 8.53-8.72 (1H, m), 7.95-8.08 (2H, m), 7.54-7.69 (1H, m), 4.57 (2H, d, J5.6), 3.06-3.24 (3H, m), 2.67-2.68 (2H, m), 2.33-2.38 (2H, m), 2.12-2.25 (2H, m), 1.80-1.97 (2H, m), 1.09 (3H, t, J 7.1).

### Example 278: 2-(3,8-diazabicyclo[3.2.1]octan-8-yl)-N-(2-isopropoxyethyl)benzo[d]thiazole-6-carboxamide

Example 278 may be prepared by analogy to steps 3 to 6 of Scheme 17 but commencing with ethyl 2-bromobenzo[d]thiazole-6-carboxylate and tert-butyl 3,8-diazabicyclo[3.2.1]octane-3-carboxylate in Step 3, instead of methyl 2-bromo-4-methylbenzo[d]thiazole-6-carboxylate and tert-butyl piperazine-1-carboxylate, and using 2-(propan-2-yloxy)ethylamine in Step 5 instead of 1-aminoindane. The title compound was isolated as the free base. ¹H NMR δ_{H} (400 MHz, DMSO-d₆) 8.36-8.43 (1H, m), 8.21-8.26 (1H, m), 7.74-7.81 (1H, m), 7.42-7.47 (1H, m), 4.19-4.27 (2H, m), 3.52-3.63 (1H, m), 3.45-3.52 (2H, m), 3.36-3.42 (2H, m), 2.93-3.00 (2H, m), 2.59-2.66 (2H, m), 1.91-2.04 (4H, m), 1.03-1.16 (6H, m).

### Example 279: 2-(3,8-diazabicyclo[3.2.1]octan-8-yl)-N-(2-(cyclopentyloxy)ethyl)benzo[d]thiazole-6-carboxamide

Example 279 may be prepared by analogy to steps 3 to 6 of Scheme 17 but commencing with ethyl 2-bromobenzo[d]thiazole-6-carboxylate and tert-butyl 3,8-diazabicyclo[3.2.1]octane-3-carboxylate in Step 3, instead of methyl 2-bromo-4-methylbenzo[d]thiazole-6-carboxylate and tert-butyl piperazine-1-carboxylate, and using 2-(cyclopentyloxy)ethylamine in Step 5 instead of 1-aminoindane. The title compound was isolated as the free base. ¹H NMR δ_{H} (400 MHz, DMSO-d₆) 8.36-8.42 (1H, m), 8.22-8.26 (1H, m), 7.74-7.80 (1H, m), 7.42-7.47 (1H, m), 4.19-4.28 (2H, m), 3.87-3.94 (1H, m), 3.43-3.50 (2H, m), 3.36-3.42 (2H, m), 2.91-3.01 (2H, m), 2.60-2.67 (2H, m), 1.90-2.08 (4H, m), 1.30-1.79 (8H, m).

### Example 280: N-cyclopentyl-2-(1,2-dimethyl-1H-imidazol-5-yl)benzo[d]thiazole-6-carboxamide

Example 280 may be prepared according to Scheme 1, using 1,2-dimethyl-5-(4,4,5,5-tetramethyl-1,3,2-dioxaborolan-2-yl)-1H-imidazole instead of (2-methylpyridin-3-yl)boronic acid in Step 2 and cyclopentylamine as the amine component. ¹H NMR δ_{H} (400 MHz, DMSO-d₆) 8.49-8.61 (1H, m), 8.33-8.47 (1H, m), 7.92-8.09 (2H, m), 7.67 (1H, s), 4.17-4.36 (1H, m), 4.12 (3H, s), 2.42 (3H, s), 1.81-2.03 (2H, m), 1.63-1.80 (2H, m), 1.47-1.63 (4H, m).

### Example 281: N-cyclopentyl-2-(1-ethylazetidin-3-yl)benzo[d]thiazole-6-carboxamide

Example 281 may be prepared according to Scheme 29:

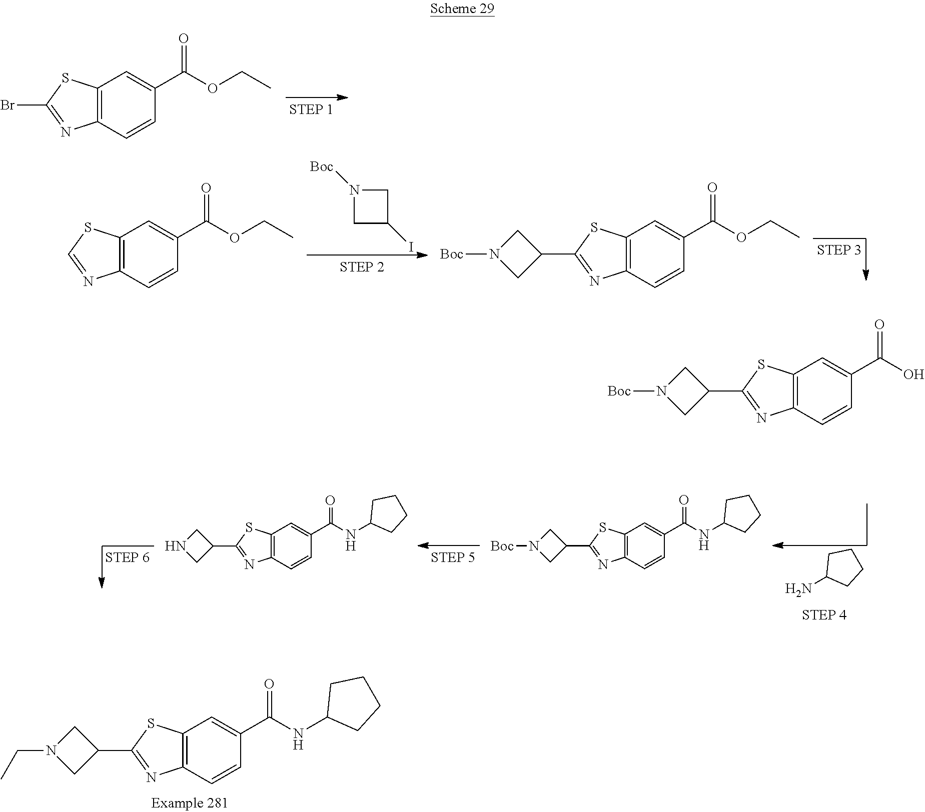

### Step 1 (Scheme 29): Synthesis of ethyl benzo[d]thiazole-6-carboxylate

To a stirred solution of ethyl 2-bromobenzo[d]thiazole-6-carboxylate (8.0 g, 28.0 mmol) in ethanol (80 mL) under nitrogen was added Pd/C (10% Pd on carbon; 1.49 g, 1.40 mmol). The reaction mixture was stirred at r.t. for 16 h under hydrogen (bladder pressure, 14 psi), then filtered through Celite®. The filtrate was concentrated under reduced pressure to afford ethyl benzo[d]thiazole-6-carboxylate (6.5 g, 80%) as a light yellow solid.

### Step 2 (Scheme 29): Synthesis of ethyl 2-(1-(tert-butoxycarbonyl)azetidin-3-yl) enzob[d]thiazole-6-carboxylate

A stirred solution of ethyl benzo[d]thiazole-6-carboxylate (4.0 g, 19.3 mmol) and potassium persulfate (10.4 g, 38.6 mmol) in DMSO (24 mL) and water (8 mL) in a 100 mL sealed tube was purged for 5 min with nitrogen prior to addition of tert-butyl 3-iodoazetidine-1-carboxylate (10.9 g, 38.6 mmol) followed by DIPEA (13.5 ml, 77 mmol). The reaction mixture was stirred at 100° C. for 16 h, then diluted with cold water and extracted with ethyl acetate. The organic layer washed with brine, dried over anhydrous sodium sulfate, filtered and concentrated under reduced pressure. The residue was purified by flash chromatography, eluting with 15-20% ethyl acetate in petroleum ether, to afford ethyl 2-(1-(tert-butoxycarbonyl)azetidin-3-yl)benzo[d]thiazole-6-carboxylate (1.1 g, 14.8%) as a yellow gummy solid.

### Step 3 (Scheme 29): Synthesis of 2-(1-(tert-butoxycarbonyl)azetidin-3-yl)benzo[d]thiazole-6-carboxylic acid

To stirred solution of ethyl 2-(1-(tert-butoxycarbonyl)azetidin-3-yl)benzo[d]thiazole-6-carboxylate (600 mg, 1.66 mmol) in 1:1 THF:H₂O (10 mL) was added LiOH·H₂O (139 mg, 3.31 mmol). The reaction mixture was stirred at r.t. for 16 h, then diluted with cold water and extracted with DCM. The organic layer was separated, washed with brine, dried over anhydrous sodium sulfate, filtered and concentrated under reduced pressure to afford 2-(1-(tert-butoxycarbonyl)azetidin-3-yl)benzo[d]thiazole-6-carboxylic acid (0.47 g, 71%) as an off-white solid.

### Step 4 (Scheme 29): Synthesis of tert-butyl 3-(6-(cyclopentylcarbamoyl)benzo[d]thiazol-2-yl)azetidine-1-carboxylate

To a stirred solution of 2-(1-(tert-butoxycarbonyl)azetidin-3-yl)benzo[d]thiazole-6-carboxylic acid (500 mg, 1.5 mmol) in DCM (10 mL) at 0° C. was added DIPEA (1.05 mL, 6.0 mmol) and n-propylphosphonic acid anhydride, cyclic trimer (50% in ethyl acetate; 1.8 mLl, 3.0 mmol). Cyclopentanamine (0.22 mL, 2.24 mmol) was added and the mixture was stirred at r.t. for 16 h, then diluted with DCM and washed with water and brine. The organic layer was separated, dried over anhydrous sodium sulfate, filtered and concentrated under reduced pressure to afford tert-butyl 3-(6-(cyclopentylcarbamoyl)benzo[d]thiazol-2-yl)azetidine-1-carboxylate (0.64 g, 98%) as an off-white solid.

### Step 5 (Scheme 29): Synthesis of 2-(azetidin-3-yl)-N-cyclopentylbenzo[d]thiazole-6-carboxamide

To stirred solution of tert-butyl 3-(6-(cyclopentylcarbamoyl)benzo[d]thiazol-2-yl)azetidine-1-carboxylate (750 mg, 1.87 mmol) in DCM (5 mL) was added trifluoroacetic acid (0.43 mL, 5.6 mmol). The mixture was stirred at r.t. for 16 h, then concentrated under reduced pressure to afford 2-(azetidin-3-yl)-N-cyclopentylbenzo[d]thiazole-6-carboxamide (0.45 g, 74%) as a yellow gum.

### Step 6 (Scheme 29): Synthesis of N-cyclopentyl-2-(1-ethylazetidin-3-yl)benzo[d]thiazole-6-carboxamide

To a stirred solution of 2-(azetidin-3-yl)-N-cyclopentylbenzo[d]thiazole-6-carboxamide (350 mg, 1.16 mmol) in methanol (3 mL) at 0° C. was added acetic acid (0.13 mL, 2.32 mmol) followed by acetaldehyde (0.20 mL, 3.48 mmol). The mixture was stirred at 0° C. for 2 h. Sodium cyanoborohydride (146 mg, 2.32 mmol) was added and the mixture stirred at r.t. for 16 h, then concentrated under reduced pressure. The residue was diluted with cold water and extracted with 10% methanol in DCM (2×). The combined organic layers were washed with brine, dried over anhydrous sodium sulfate, filtered and concentrated under reduced pressure. The crude product was purified by preparative HPLC using ammonium bicarbonate as buffer to afford N-cyclopentyl-2-(1-ethylazetidin-3-yl)benzo[d]thiazole-6-carboxamide (20 mg, 5%) as an off-white solid. ¹H NMR δ_{H} (400 MHz, DMSO-d₆) 8.55 (1H, d, J 1.0), 8.40 (1H, d, J7.2), 7.94-8.01 (2H, m), 4.18-4.33 (1H, m), 4.00-4.16 (1H, m), 3.65 (2H, br t, J7.5), 2.45-2.51 (4H, m), 1.80-2.01 (2H, m), 1.64-1.80 (2H, m), 1.51-1.71 (4H, m), 0.91 (3H, t, J7.2).

### Example 282: rac-N-cyclopentyl-2-((3S,4R)-3-hydroxy-1-methylpiperidin-4-yl)benzo[d]thiazole-6-carboxamide

Example 282 may be prepared according to Scheme 30:

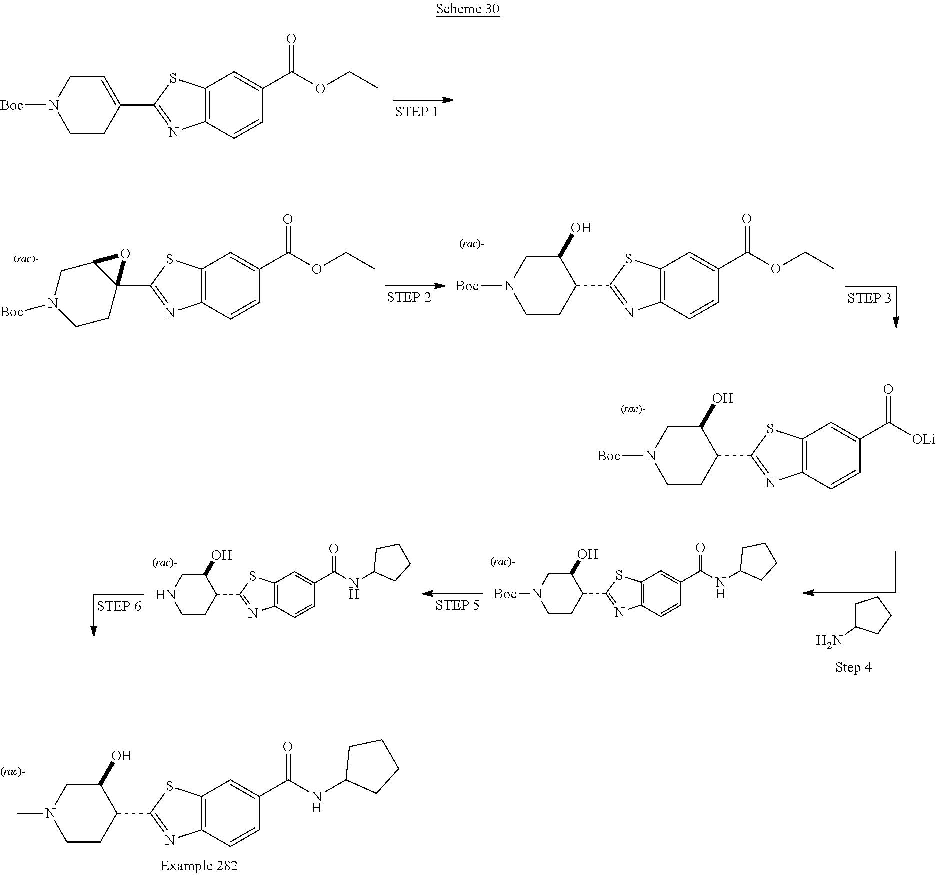

### Step 1 (Scheme 30): tert-butyl 6-(6-(ethoxycarbonyl)benzo[d]thiazol-2-yl)-7-oxa-3-azabicyclo[4.1.0]heptane-3-carboxylate

To a stirred solution of ethyl 2-(1-(tert-butoxycarbonyl)-1,2,3,6-tetrahydropyridin-4-yl)benzo[d]thiazole-6-carboxylate (prepared as described in Scheme 9, Step 1; 3 g, 7.72 mmol) in DCM (25 mL) at 0° C. was added m-CPBA (4.70 g, 23.2 mmol) under a continuous stream of nitrogen over 10 minutes. The reaction mixture was stirred at r.t. for 48 h, then quenched with Na₂S₂O₃ solution (50 mL) and extracted with ethyl acetate (2×50 mL). The combined organic layers were washed with 10% NaHCO₃ solution (75 mL), brine (75 mL), dried over anhydrous sodium sulfate, filtered and concentrated under reduced pressure. The residue was purified by flash chromatography, eluting with 33% ethyl acetate in petroleum ether, to obtain tert-butyl 6-(6-(ethoxycarbonyl)benzo[d]thiazol-2-yl)-7-oxa-3-azabicyclo[4.1.0]heptane-3-carboxylate (720 mg, 20%) as an off-white solid.

### Step 2 (Scheme 30): ethyl 2-(1-(tert-butoxycarbonyl)-3-hydroxypiperidin-4-yl)benzo[d]thiazole-6-carboxylate

To a stirred solution of tert-butyl 6-(6-(ethoxycarbonyl)benzo[d]thiazol-2-yl)-7-oxa-3-azabicyclo[4.1.0]heptane-3-carboxylate (1.2 g, 3.0 mmol) in ethanol (15 mL) and acetic acid (0.89 g, 14.8 mmol) under nitrogen was added Pd/C (10% Pd on carbon; 0.32 g, 3.0 mmol) over 5 min. The mixture was stirred under hydrogen gas (1 kg/cm² pressure) at r.t. for 24 h, then filtered through Celite®, washing with methanol (50 mL). The combined filtrate was dried over anhydrous sodium sulfate, filtered and concentrated under reduced pressure. The residue was purified by flash chromatography, eluting with 40% ethyl acetate in petroleum ether, to obtain ethyl 2-(1-(tert-butoxycarbonyl)-3-hydroxypiperidin-4-yl)benzo[d]thiazole-6-carboxylate (650 mg, 45%).

### Step 3 (Scheme 30): lithium 2-(1-(tert-butoxycarbonyl)-3-hydroxypiperidin-4-yl)benzo[d]thiazole-6-carboxylate

To a stirred solution of ethyl 2-(1-(tert-butoxycarbonyl)-3-hydroxypiperidin-4-yl)benzo[d]thiazole-6-carboxylate (100 mg, 0.25 mmol) in a mixture of methanol (0.3 mL), THF (0.6 mL) and water (0.1 mL) was added LiOH·H₂O (41.3 mg, 0.98 mmol). The mixture was stirred at r.t. for 16 h, then concentrated under reduced pressure to obtain lithium 2-(1-(tert-butoxycarbonyl)-3-hydroxypiperidin-4-yl)benzo[d]thiazole-6-carboxylate (105 mg, 86%) as an off-white solid.

### Step 4 (Scheme 30): tert-butyl 4-(6-(cyclopentylcarbamoyl)benzo[d]thiazol-2-yl)-3-hydroxypiperidine-1-carboxylate

To a stirred solution of lithium 2-(1-(tert-butoxycarbonyl)-3-hydroxypiperidin-4-yl)benzo[d]thiazole-6-carboxylate (90 mg, 0.23 mmol) in DCM (1 mL) at 0° C. was added n-propylphosphonic acid anhydride, cyclic trimer (50% in EtOAc; 300 mg, 0.47 mmol) and N,N-diisopropylethylamine (150 mg, 1.17 mmol). After 15 minutes, cyclopentanamine (21.9 mg, 0.26 mmol) was added. The mixture was stirred at r.t. for 18 h, then quenched with water (10 mL) and extracted with ethyl acetate (2×10 mL). The combined organic layers were washed with 10% NaHCO₃ solution (10 mL), brine (15 mL), dried over anhydrous sodium sulfate, filtered and concentrated under reduced pressure. The residue was purified by flash chromatography, eluting with 50% ethyl acetate in petroleum ether to obtain tert-butyl 4-(6-(cyclopentylcarbamoyl)benzo[d]thiazol-2-yl)-3-hydroxypiperidine-1-carboxylate (76 mg, 73%) as an off-white solid.

### Step 5 (Scheme 30): N-cyclopentyl-2-(3-hydroxypiperidin-4-yl)benzo[d]thiazole-6-carboxamide hydrochloride

To a stirred solution of tert-butyl 4-(6-(cyclopentylcarbamoyl)benzo[d]thiazol-2-yl)-3-hydroxypiperidine-1-carboxylate (75 mg, 0.17 mmol) in DCM (2 mL) at 0° C. was added HCl (4.0 M in dioxane; 0.17 mL, 0.68 mmol). The mixture was stirred at r.t. for 1 6h, then concentrated under reduced pressure to obtain crude N-cyclopentyl-2-(3-hydroxypiperidin-4-yl)benzo[d]thiazole-6-carboxamide hydrochloride (65 mg, 81%), which was used directly in the next step.

### Step 6 (Scheme 30): N-cyclopentyl-2-(3-hydroxy-1-methylpiperidin-4-yl)benzo[d]thiazole-6-carboxamide

To a solution of N-cyclopentyl-2-(3-hydroxypiperidin-4-yl)benzo[d]thiazole-6-carboxamide hydrochloride (60 mg, 0.16 mmol) in methanol (2 mL) under nitrogen at 0° C. was added formaldehyde (37% w/w in water; 19.1 mg, 0.24 mmol) and acetic acid (0.94 mg, 0.016 mmol). The mixture was stirred at r.t. for 2 h, then cooled to 0° C. and sodium cyanotrihydroborate (11.9 mg, 0.19 mmol) was added. The mixture was stirred at r.t. for 16 h, then concentrated under reduced pressure. The residue was purified by preparative HPLC to afford the title compound (20 mg, 35%) as an off-white solid. ¹H NMR δ_{H} (400 MHz, DMSO-d₆) 8.53 (1H, d, J 0.9), 8.35-8.46 (1H, m), 7.86-8.05 (2H, m), 6.00-6.20 (1H, m), 5.05-5.37 (1H, m), 4.17-4.36 (1H, m), 3.66-3.83 (1H, m), 2.92-3.05 (1H, m), 2.78-2.92 (2H, m), 2.22 (3H, s), 2.02-2.15 (1H, m), 1.78-2.00 (4H, m), 1.66-1.78 (2H, m), 1.50-1.62 (4H, m).

### Example 283: N-cyclopentyl-2-(piperidin-4-yl)benzo[d]thiazole-5-carboxamide

Example 283 may be prepared according to Scheme 31:

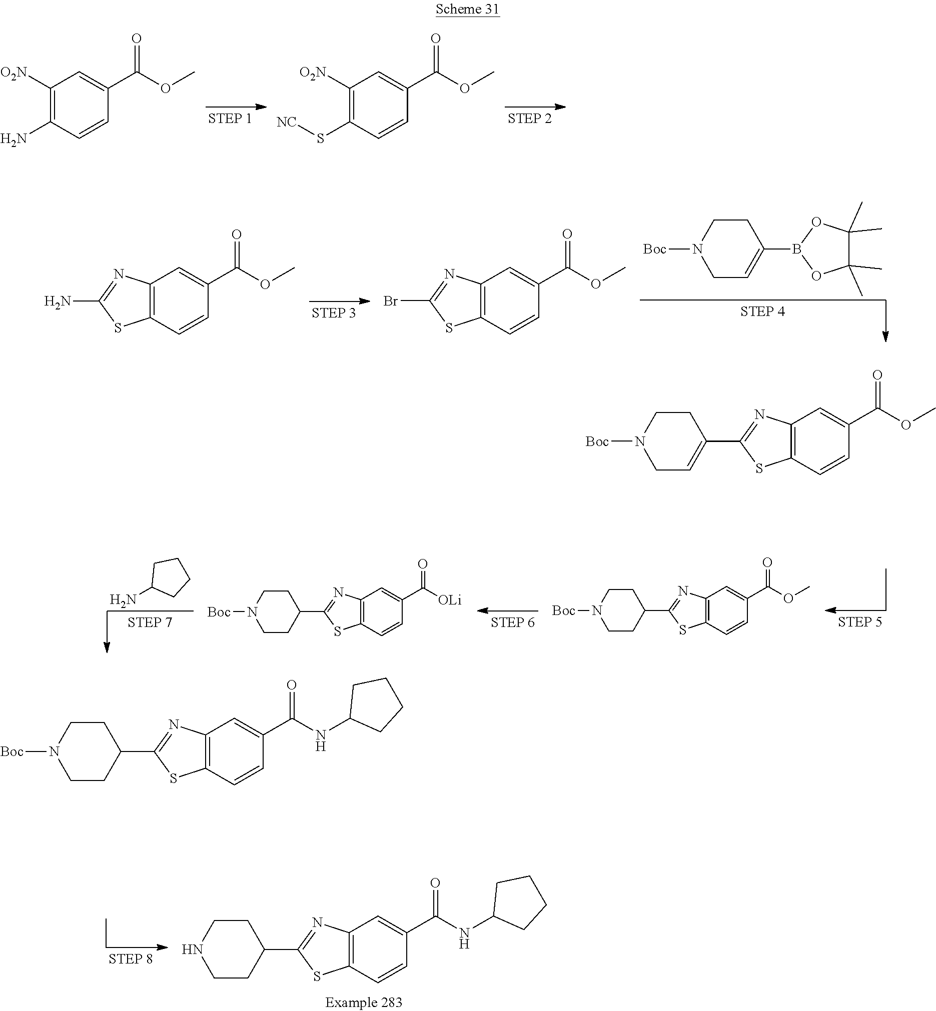

### Step 1 (Scheme 31): Methyl 3-nitro-4-thiocyanatobenzoate

To a stirred solution of methyl 4-amino-3-nitrobenzoate (5 g, 26 mmol) in acetonitrile (60 mL) at 0° C. was added tert-butyl nitrite (7.9 g, 76 mmol). The mixture was stirred for 30 minutes, then allowed to warm to r.t. Potassium thiocyanate (9.9 g, 100 mmol) was added lot-wise over 2 h. The mixture was stirred at r.t. for 4 h, then diluted with ethyl acetate (50 mL), washed with water (50 mL) and brine (50 mL), dried over sodium sulfate, filtered and concentrated under reduced pressure. The residue was purified by flash chromatography, eluting with 12% ethyl acetate in petroleum ether to obtain methyl 3-nitro-4-thiocyanatobenzoate (870 mg, 14%) as a light yellow solid.

### Step 2 (Scheme 31): Methyl 2-aminobenzo[d]thiazole-5-carboxylate

A stirred solution of methyl 3-nitro-4-thiocyanatobenzoate (1 g, 4.2 mmol) in acetic acid (15 mL) was purged with nitrogen gas for 10 min. To this mixture was added Pd/C (10% Pd on carbon; 0.4 g, 3.8 mmol) over 5 minutes under nitrogen. The mixture was stirred under hydrogen (4 kg/cm² pressure) at r.t. for 40 h, then filtered through Celite®, washing with ethyl acetate. The combined filtrate was concentrated under reduced pressure. The residue was triturated with methyl tert-butyl ether and the solid filtered off and dried to obtain methyl 2-aminobenzo[d]thiazole-5-carboxylate (810 mg, 69%) as a light brown solid.

Steps 3 to 8 (Scheme 31): Methyl 2-aminobenzo[d]thiazole-5-carboxylate from the preceding step was converted into N-cyclopentyl-2-(piperidin-4-yl)benzo[d]thiazole-5-carboxamide according to Steps 3 to 8 of Scheme 31, following the procedures described for Steps 2 to 7 of Scheme 16 but using cyclopentylamine in place of 1-aminoindane. ¹H NMR δ_{H} (400 MHz, DMSO-d₆) 8.44-8.50 (1H, m), 8.37-8.45 (1H, m), 8.09-8.17 (1H, m), 7.89 (1H, dd, J 8.4, 1.3), 4.16-4.34 (1H, m), 3.02-3.16 (2H, m), 2.61-2.79 (2H, m), 2.01-2.12 (2H, m), 1.84-1.97 (3H, m), 1.62-1.82 (5H, m), 1.46-1.63 (4H, m).

### Example 284: 2-(3,8-diazabicyclo[3.2.1]octan-8-yl)-N-cyclopentylbenzo[d]thiazole-5-carboxamide

Methyl 2-aminobenzo[d]thiazole-5-carboxylate (prepared as in Step 2 of Scheme 31) was converted into the title compound following the procedures described in Steps 3 to 6 of Scheme 17, using tert-butyl 3,8-diazabicyclo[3.2.1]octane-3-carboxylate instead of tert-butyl piperazine-1-carboxylate in Step 3 and cyclopentylamine instead of 1-aminoindane in Step 5. The title compound was isolated as the hydrochloride salt. ¹H NMR δ_{H} (400 MHz, DMSO-d₆) 9.27-9.44 (1H, m), 8.98-9.16 (1H, m), 8.20-8.40 (1H, m), 8.01-8.14 (1H, m), 7.86-7.96 (1H, m), 7.58-7.71 (1H, m), 4.44-4.60 (2H, m), 4.17-4.33 (1H, m), 3.17-3.33 (4H, m), 2.01-2.25 (4H, m), 1.80-1.95 (2H, m), 1.64-1.78 (2H, m), 1.43-1.62 (4H, m).

### Example 285: N-cyclopentyl-2-(3-methyl-3,8-diazabicyclo[3.2.1]octan-8-yl)benzo[d]thiazole-5-carboxamide

Example 285 may be prepared by reductive alkylation of 2-(3,8-diazabicyclo[3.2.1]octan-8-yl)-N-cyclopentylbenzo[d]thiazole-5-carboxamide (Example 284) with formaldehyde as described for Examples 260 to 266. ¹H NMR δ_{H} (400 MHz, DMSO-d₆) 8.22-8.30 (1H, m), 7.99 (1H, d, J 1.3), 7.78-7.86 (1H, m), 7.51-7.62 (1H, m), 4.17-4.37 (3H, m), 2.63-2.74 (2H, m), 2.29-2.37 (2H, m), 2.15 (3H, s), 1.80-2.02 (6H, m), 1.64-1.77 (2H, m), 1.46-1.63 (4H, m).

### Example 286: N-cyclopentyl-2-(4-ethylpiperazin-1-yl)benzo[d]thiazole-6-sulfonamide

Example 286 may be prepared according to Scheme 32:

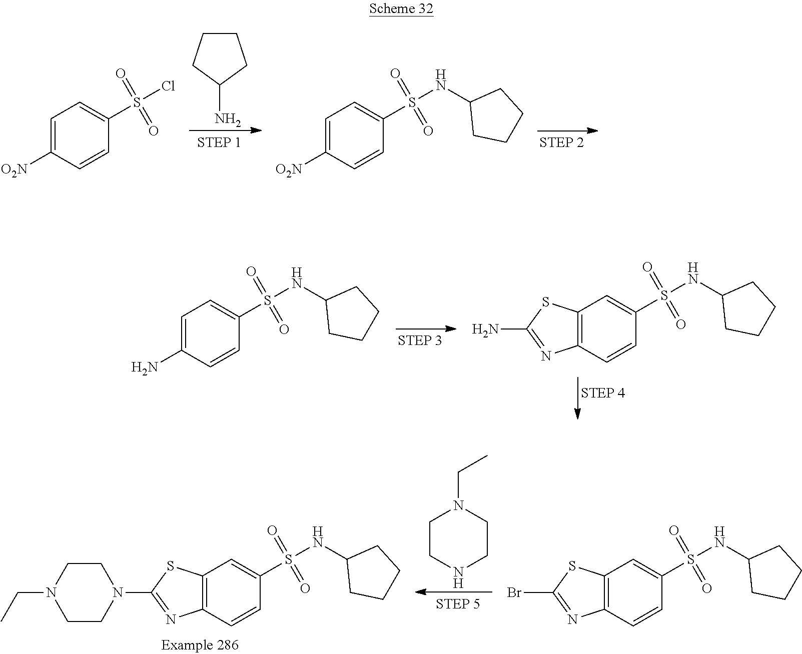

### Step 1 (Scheme 32): Synthesis of N-cyclopentyl-4-nitrobenzenesulfonamide

To a stirred solution of 4-nitrobenzenesulfonyl chloride (1.0 g, 4.5 mmol) in DCM (10 mL) at 0° C. were added N,N-diisopropylethylamine (1.57 mL, 9.0 mmol) and cyclopentanamine (0.77 g, 9.0 mmol). The mixture was stirred at r.t. for 16 h, then diluted with DCM (50 mL), washed with water (25 mL) and brine (25 mL) and concentrated under reduced pressure. The residue was triturated with 10% ethyl acetate in hexane and the solid filtered off to obtain N-cyclopentyl-4-nitrobenzenesulfonamide (900 mg, 98%) as an light brown solid.

### Step 2 (Scheme 32): Synthesis of 4-amino-N-cyclopentylbenzenesulfonamide

To a stirred solution N-cyclopentyl-4-nitrobenzenesulfonamide (10 g, 37.0 mmol) in ethanol (50 mL) and water (50 mL) at 0° C. was added zinc (24.2 g, 370 mmol) and ammonium chloride (9.9 g, 190 mmol). The mixture was stirred at 100° C. for 16 h, then allowed to cool, filtered through Celite®, diluted with water (500 mL) and extracted with DCM (2×500 mL). The combined organic layers were washed with water and brine, dried over sodium sulfate and concentrated under reduced pressure to afford 4-amino-N-cyclopentylbenzenesulfonamide (7.0 g, 87%) as an off-white solid.

### Step 3 (Scheme 32): Synthesis of 2-amino-N-cyclopentylbenzo[d]thiazole-6-sulfonamide

To a stirred mixture of 4-amino-N-cyclopentylbenzenesulfonamide (20 g, 83 mmol) and potassium thiocyanate (32.3 g, 330 mmol) in AcOH (200 mL) at 0° C. was added dropwise a solution of dibromine (4.28 ml, 83 mmol) in AcOH (100 mL). After 1 h (ca. half of the Br₂ solution addition) stirring failed and the mixture was allowed to warm to ambient temperature.

The remaining half of the Br₂ solution was added over a further period of 1 h. The resulting yellow slurry was then heated to 40° C. for 16 h, then cooled, diluted with ice-water (1000 mL) and adjusted to pH 9 with ice-chilled 2M NaOH solution. The resulting yellow precipitate was collected by filtration, washing with water, and dried to afford 2-amino-N-cyclopentylbenzo[d]thiazole-6-sulfonamide (24.0 g, 92%) as a yellow solid.

### Step 4 (Scheme 32): Synthesis of 2-bromo-N-cyclopentylbenzo[d]thiazole-6-sulfonamide

To a stirred solution of copper(II) bromide (34.2 g, 150 mmol) in acetonitrile (1000 mL) at 0° C. was added dropwise tert-butyl nitrite (15.8 g, 150 mmol). After 20 minutes, 2-amino-N-cyclopentylbenzo[d]thiazole-6-sulfonamide (24 g, 81 mmol) was added. The mixture was allowed to warm to r.t. and stirred for 16 h, then filtered through Celite®, washing with DCM. The combined filtrate was concentrated under reduced pressure. The residue was diluted with DCM, washed with (1.5 N) aqueous hydrochloric acid followed by water and brine, dried over anhydrous sodium sulfate, filtered and concentrated under reduced pressure to afford 2-bromo-N-cyclopentylbenzo[d]thiazole-6-sulfonamide (24 g, 77%) as a yellow solid.

### Step 5 (Scheme 32): Synthesis of N-cyclopentyl-2-(4-ethylpiperazin-1-yl)benzo[d]thiazole-6-sulfonamide

To a stirred solution of 2-bromo-N-cyclopentylbenzo[d]thiazole-6-sulfonamide (500 mg, 1.38 mmol) in acetonitrile (25 mL) was added potassium carbonate (383 mg, 2.77 mmol) and 1-ethylpiperazine (174 mg, 1.52 mmol). The mixture was stirred at 80° C. for 16 h, then diluted with water and extracted with DCM (2×250 mL). The combined organic layers were washed with water and brine, dried over sodium sulfate, filtered and concentrated under reduced pressure. The residue was purified by flash chromatography, eluting with 8-10% methanol in DCM, to afford N-cyclopentyl-2-(4-ethylpiperazin-1-yl)benzo[d]thiazole-6-sulfonamide as an off-white solid (320 mg, 58%). ¹H NMR δ_{H} (400 MHz, DMSO-d₆) b ppm 8.24 (1H, d, J 1.8), 7.65-7.70 (1H, m), 7.53-7.59 (1H, m), 7.48-7.52 (1H, m), 3.58-3.66 (4H, m), 3.33-3.44 (1H, m), 2.42-2.37 (6H, m), 1.46-1.61 (4H, m), 1.18-1.44 (4H, m), 1.04 (3H, t, J7.2).

Examples 287 and 288 Examples 287 and 288 may be prepared according to Scheme 33:

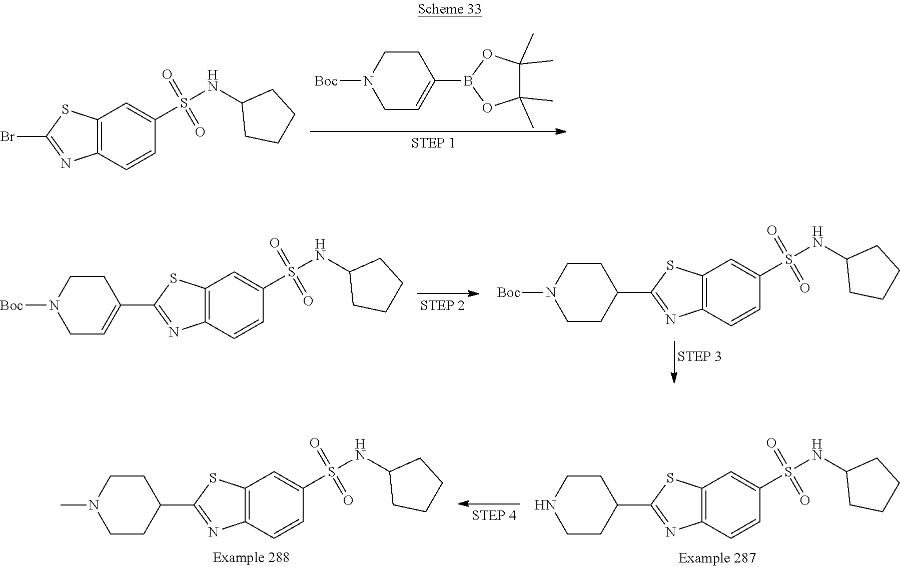 [SMILES: O=S(=O)(NC1CCCC1)c1ccc2nc(Br)sc2c1 ; CC(C)(C)OC(=O)N1CC=C(B2OC(C)(C)C(C)(C)O2)CC1 ; CC(C)(C)OC(=O)N1CC=C(c2nc3ccc(S(=O)(=O)NC4CCCC4)cc3s2)CC1 ; CC(C)(C)OC(=O)N1CCC(c2nc3ccc(S(=O)(=O)NC4CCCC4)cc3s2)CC1 ; CN1CCC(c2nc3ccc(S(=O)(=O)NC4CCCC4)cc3s2)CC1 ; O=S(=O)(NC1CCCC1)c1ccc2nc(C3CCNCC3)sc2c1]

Example 287:N-cyclopentyl-2-(piperidin-4-yl)benzo[d]thiazole-6-sulfonamide

### Step 1 (Scheme 33): Synthesis of tert-butyl 4-(6-(N-cyclopentylsulfamoyl)benzo[d]thiazol-2-yl)-3,6-dihydropyridine-1 (2H)-carboxylate

To a degassed solution of 2-bromo-N-cyclopentylbenzo[d]thiazole-6-sulfonamide (4.0 g, 11.1 mmol) in 1,4-dioxane (45 mL) and water (5 mL) under argon at r.t. was added tert-butyl 4-(4,4,5,5-tetramethyl-1,3,2-dioxaborolan-2-yl)-3,6-dihydropyridine-1 (2H)-carboxylate (3.42 g, 11.1 mmol), K₂CO₃ (3.06 g, 22.1 mmol) and Pd(PPh₃)₄ (1.28 g, 1.11 mmol). The mixture was stirred at 100° C. for 5 h, then cooled to r.t., diluted with ethyl acetate and filtered through Celite®, washing with ethyl acetate. The combined filtrate was concentrated under reduced pressure. The residue was purified by flash chromatography, eluting with 20-30% ethyl acetate in petroleum ether, to afford tert-butyl 4-(6-(N-cyclopentylsulfamoyl)benzo[d]thiazol-2-yl)-3,6-dihydropyridine-1 (2H)-carboxylate (2.9 g, 51%) as an off-white solid.

### Step 2 (Scheme 33): Synthesis of tert-butyl 4-(6-(N-cyclopentylsulfamoyl)benzo[d]thiazol-2-yl)piperidine-1-carboxylate

To a stirred solution of tert-butyl 4-(6-(N-cyclopentylsulfamoyl)benzo[d]thiazol-2-yl)-3,6-dihydropyridine-1 (2H)-carboxylate (2.8 g, 6.04 mmol) in ethanol (70 mL) under nitrogen was added platinum(IV) oxide (1.37 g, 6.04 mmol). The mixture was stirred at r.t. under H₂ pressure (20 psi) for 16 h, then filtered through Celite®, washing with ethyl acetate. The combined filtrate was concentrated under reduced pressure to afford tert-butyl 4-(6-(N-cyclopentylsulfamoyl)benzo[d]thiazol-2-yl)piperidine-1-carboxylate (3.0 g, 79%) as a brown solid.

### Step 3 (Scheme 33): Synthesis of N-cyclopentyl-2-(piperidin-4-yl)benzo[d]thiazole-6-sulfonamide

To a stirred solution of tert-butyl 4-(6-(N-cyclopentylsulfamoyl)benzo[d]thiazol-2-yl) piperidine-1-carboxylate (2.9 g, 6.23 mmol) in DCM (30 mL) was added HCl (4.0 M in dioxane; 3.11 mL, 12.5 mmol). The mixture was stirred at r.t. for 16 h, then concentrated under reduced pressure to afford crude N-cyclopentyl-2-(piperidin-4-yl)benzo[d]thiazole-6-sulfonamide (2.5 g), of which 220 mg was purified by preparative HPLC using ammonium bicarbonate as buffer to afford N-cyclopentyl-2-(piperidin-4-yl)benzo[d]thiazole-6-sulfonamide (23 mg, 51%) as an off-white solid. ¹H NMR δ_{H} (400 MHz, DMSO-d₆) 8.54-8.62 (1H, m), 8.09-8.15 (1H, m), 7.82-7.93 (1H, m), 7.68-7.78 (1H, m), 3.21-3.32 (1H, m), 2.99-3.08 (2H, m), 2.56-2.73 (3H, m), 2.00-2.11 (2H, m), 1.43-1.77 (6H, m), 1.18-1.44 (4H, m).

### Example 288: N-cyclopentyl-2-(1-methylpiperidin-4-yl)benzo[d]thiazole-6-sulfonamide Step 4 (Scheme 33)

To a stirred solution of N-cyclopentyl-2-(piperidin-4-yl)benzo[d]thiazole-6-sulfonamide (500 mg, 1.37 mmol) in methanol (15 mL) at 0° C. was added acetic acid (0.16 mL, 2.74 mmol) followed by formaldehyde (37% in water; 0.41 mL, 4.10 mmol). The mixture was stirred at r.t. for 2 h, then cooled to 0° C. and sodium borohydride (207 mg, 5.47 mmol) was added. The mixture was stirred at r.t. for 5 h, then quenched with ice-cold water and extracted with ethyl acetate. The combined organic extracts were washed with brine, separated, dried over anhydrous sodium sulfate, filtered and concentrated under reduced pressure. The residue was purified by preparative HPLC using ammonium bicarbonate as buffer to afford N-cyclopentyl-2-(1-methylpiperidin-4-yl)benzo[d]thiazole-6-sulfonamide (280 mg, 53%) as an off-white solid. ¹H NMR δ_{H} (400 MHz, DMSO-d₆) 8.58 (1H, d, J 1.6), 8.07-8.18 (1H, m), 7.81-7.90 (1H, m), 7.69-7.78 (1H, m), 3.37-3.50 (1H, m), 3.06-3.19 (1H, m), 2.78-2.92 (2H, m), 2.21 (3H, s), 2.01-2.14 (4H, m), 1.75-1.91 (2H, m), 1.46-1.63 (4H, m), 1.22-1.41 (4H, m).

Examples 289 and 290

Examples 289 and 290 may be prepared according to Scheme 34:

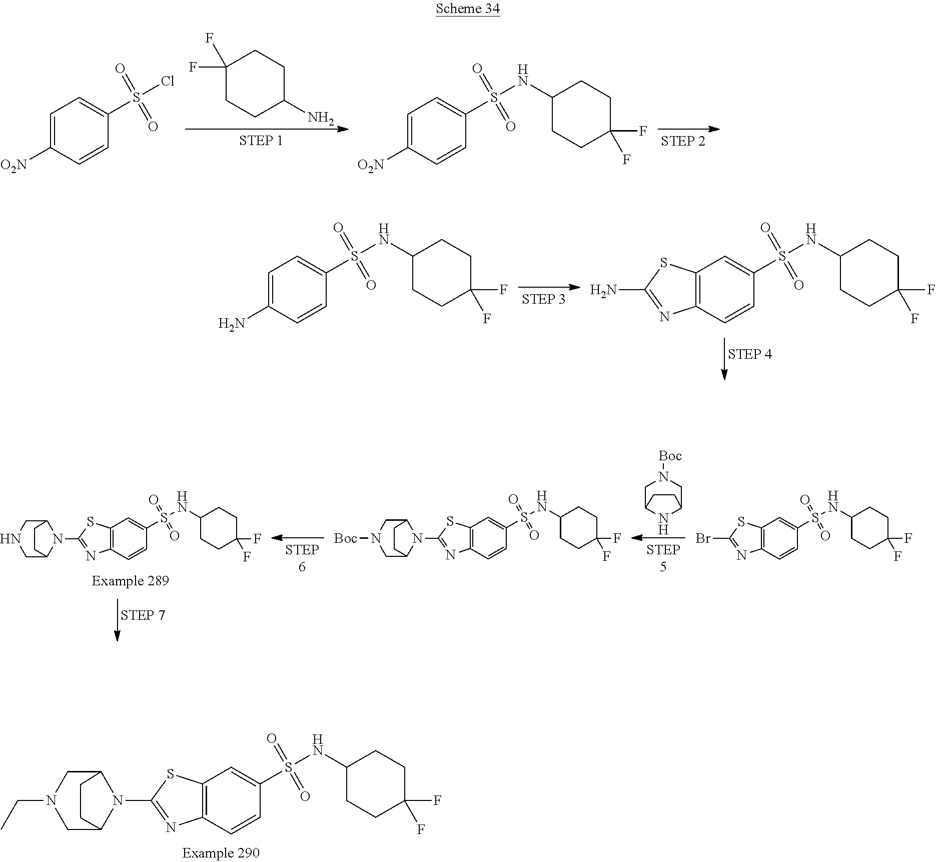

### Example 289: 2-(3,8-diazabicyclo[3.2.1]octan-8-yl)-N-(4,4-difluorocyclohexyl)benzo[d]thiazole-6-sulfonamide

### Steps 1 to 4 (Scheme 34): Synthesis of 2-bromo-N-(4,4-difluorocyclohexyl)benzo[d]thiazole-6-sulfonamide

Prepared following the procedures described for Steps 1 to 4 of Scheme 32 but using 4.4-difluorocyclohexylamine instead of cyclopentylamine.

### Step 5 (Scheme 34): Synthesis of tert-butyl 8-(6-(N-(4,4-difluorocyclohexyl) sulfamoyl)benzo[d]thiazol-2-yl)-3,8-diazabicyclo[3.2.1]octane-3-carboxylate

To a stirred solution of 2-bromo-N-(4,4-difluorocyclohexyl)benzo[d]thiazole-6-sulfonamide (1.0 g, 2.43 mmol, 1.0 equiv.) in acetonitrile (25 mL) was added potassium carbonate (0.672 g, 4.86 mmol, 2.0 equiv.) and tert-butyl 3,8-diazabicyclo[3.2.1]octane-3-carboxylate (1.1 equiv.). The reaction mixture was stirred at 80° C. for 16 h, then diluted with water and extracted with DCM. The combined organic extracts were washed with water and brine, dried over anhydrous sodium sulfate, filtered and concentrated under reduced pressure to afford tert-butyl 8-(6-(N-(4,4-difluorocyclohexyl)sulfamoyl)benzo[d]thiazol-2-yl)-3,8-diazabicyclo[3.2.1]octane-3-carboxylate (1.10 g, 81%) as an off-white solid.

### Step 6 (Scheme 34): Synthesis of 2-(3,8-diazabicyclo[3.2.1]octan-8-yl)-N-(4,4-difluorocyclohexyl)benzo[d]thiazole-6-sulfonamide

To a stirred solution of tert-butyl 8-(6-(N-(4,4-difluorocyclohexyl)sulfamoyl)benzo[d]thiazol-2-yl)-3,8-diazabicyclo[3.2.1]octane-3-carboxylate (1.1 g, 2.03 mmol) in DCM (20 mL) at 0° C. was added hydrogen chloride (4.0 M in 1,4-dioxane; 1.014 ml, 4.05 mmol). The reaction mixture was stirred at r.t. for 2 h, then concentrated under reduced pressure. The residue was triturated with methyl tert-butyl ether and the solid filtered off and dried to afford the title compound (840 mg, 93%) as an off-white solid. A portion of the product was further purified by preparative HPLC using ammonium bicarbonate as buffer to afford N-(4,4-difluorocyclohexyl)-2-(piperidin-4-yl)benzo[d]thiazole-6-sulfonamide as an off-white solid. 1H NMR δ_{H} (400 MHz, DMSO-d₆) 8.20-8.26 (1H, m), 7.62-7.72 (2H, m), 7.50-7.57 (1H, m), 4.19-4.31 (2H, m), 3.13-3.27 (1H, m), 2.90-3.00 (2H, m), 2.60-2.70 (2H, m), 1.72-2.03 (8H, m), 1.60-1.70 (2H, m), 1.40-1.51 (2H, m).

### Example 290: N-(4,4-difluorocyclohexyl)-2-(3-ethyl-3,8-diazabicyclo[3.2.1]octan-8-yl)benzo[d]thiazole-6-sulfonamide

### Step 7 (Scheme 34)

To a stirred solution of 2-(3,8-diazabicyclo[3.2.1]octan-8-yl)-N-(4,4-difluorocyclohexyl) benzo[d]thiazole-6-sulfonamide (250 mg, 0.57 mmol) in methanol (10 mL) at 0° C. was added acetic acid (0.065 mL, 1.13 mmol) followed by neat acetaldehyde (0.064 mL, 1.13 mmol). The mixture was stirred at r.t. for 2 h, then NaBH₃CN (107 mg, 1.7 mmol) was added. The mixture was stirred at r.t. for 16 h, then concentrated under reduced pressure, diluted with cold water and extracted with 15% methanol in DCM. The combined organic extracts were washed with brine, separated, dried over anhydrous sodium sulfate, filtered and concentrated under reduced pressure. The residue was purified by preparative HPLC using ammonium bicarbonate as buffer to afford N-(4,4-difluorocyclohexyl)-2-(3-ethyl-3,8-diazabicyclo[3.2.1]octan-8-yl)benzo[d]thiazole-6-sulfonamide (0.14 g, 53%) as an off-white solid. ¹H NMR δ_{H} (300 MHz, DMSO-d₆) 8.18-8.34 (1H, m), 7.62-7.75 (2H, m), 7.48-7.61 (1H, m), 4.32-4.44 (2H, m), 3.12-3.28 (1H, m), 2.72-2.83 (2H, m), 2.21-2.43 (5H, m), 1.72-1.96 (7H, m), 1.55-1.70 (2H, m), 1.36-1.52 (2H, m), 0.98 (3H, t, J7.1).

### Examples 291 and 292

Examples 291 and 292 may be prepared according to Scheme 35:

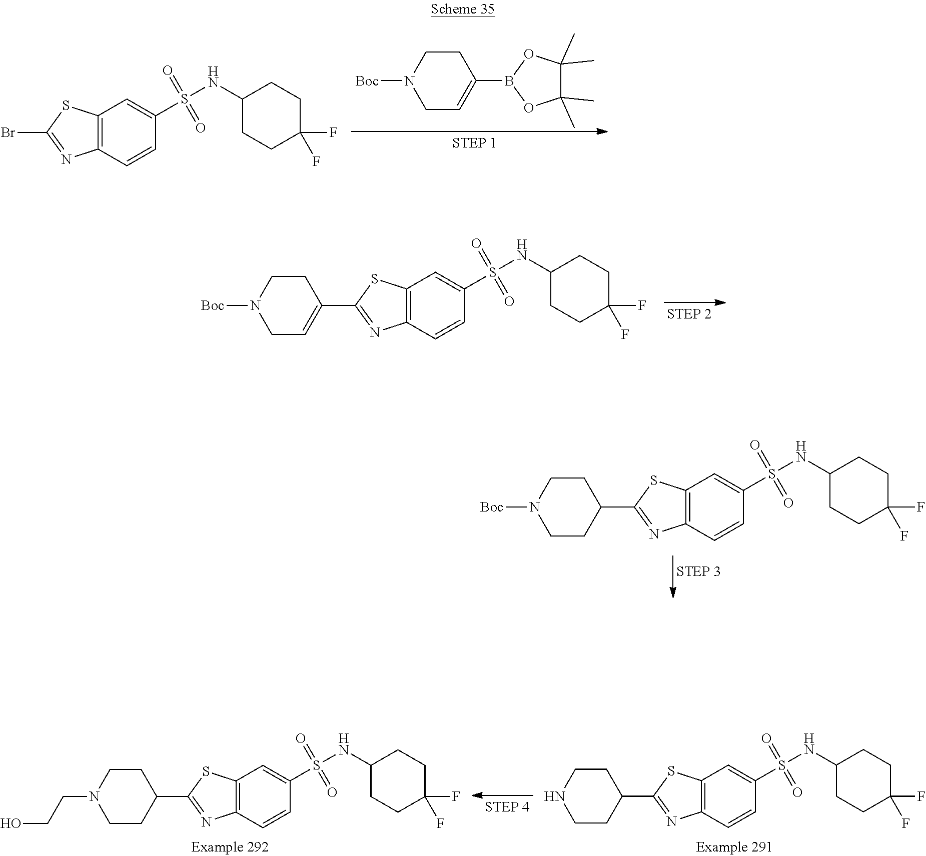 [SMILES: O=S(=O)(NC1CCC(F)(F)CC1)c1ccc2nc(Br)sc2c1 ; CC(C)(C)OC(=O)N1CC=C(B2OC(C)(C)C(C)(C)O2)CC1 ; CC(C)(C)OC(=O)N1CC=C(c2nc3ccc(S(=O)(=O)NC4CCC(F)(F)CC4)cc3s2)CC1 ; CC(C)(C)OC(=O)N1CCC(c2nc3ccc(S(=O)(=O)NC4CCC(F)(F)CC4)cc3s2)CC1 ; O=S(=O)(NC1CCC(F)(F)CC1)c1ccc2nc(C3CCN(CCO)CC3)sc2c1 ; O=S(=O)(NC1CCC(F)(F)CC1)c1ccc2nc(C3CCNCC3)sc2c1]

### Example 291: N-(4,4-difluorocyclohexyl)-2-(piperidin-4-yl)benzo[d]thiazole-6-sulfonamide

### Step 1 (Scheme 35): Synthesis of tert-butyl 4-(6-(N-(4,4-difluorocyclohexyl)sulfamoyl) benzo[d]thiazol-2-yl)-3,6-dihydropyridine-1 (2H)-carboxylate

To a stirred solution of 2-bromo-N-(4,4-difluorocyclohexyl)benzo[d]thiazole-6-sulfonamide (5 g, 12.2 mmol) in dioxane (200 mL) and water (20 mL) at r.t. under argon was added tert-butyl 4-(4,4,5,5-tetramethyl-1,3,2-dioxaborolan-2-yl)-3,6-dihydropyridine-1 (2H)-carboxylate (4.51 g, 14.6 mmol) followed by potassium carbonate (3.36 g, 24.31 mmol). The mixture was degassed for 10 min, then tetrakis(triphenylphosphine)palladium(0) (0.70 g, 0.61 mmol) was added. The mixture was stirred at 100° C. for 16 h, then allowed to cool and filtered through Celite®, washing with DCM. The combined filtrate was concentrated under reduced pressure, then purified by flash chromatography, eluting with 25-30% ethyl acetate in petroleum ether, to afford tert-butyl 4-(6-(N-(4,4-difluorocyclohexyl)sulfamoyl)benzo[d]thiazol-2-yl)-3,6-dihydropyridine-1 (2H)-carboxylate (2.70 g, 42%) as a brown solid.

### Step 2 (Scheme 35): Synthesis of tert-butyl 4-(6-(N-(4,4-difluorocyclohexyl)sulfamoyl) benzo[d]thiazol-2-yl)piperidine-1-carboxylate

A stirred solution of tert-butyl 4-(6-(N-(4,4-difluorocyclohexyl)sulfamoyl)benzo[d]thiazol-2-yl)-3,6-dihydropyridine-1 (2H)-carboxylate (2.60 g, 5.06 mmol) in ethanol (100 mL) was purged with nitrogen gas for a period of 10 min. To this mixture, platinum(IV) oxide (1.15 g, 5.06 mmol) was added over 15 minutes under nitrogen. The mixture was stirred under hydrogen (30 psi pressure) at r.t. for 16 h, then filtered through Celite®, washing with methanol. The combined filtrate was concentrated under reduced pressure to afford tert-butyl 4-(6-(N-(4,4-difluorocyclohexyl)sulfamoyl)benzo[d]thiazol-2-yl)piperidine-1-carboxylate (1.10 g, 34%) as a brown solid.

### Step 3 (Scheme 35): Synthesis of N-(4,4-difluorocyclohexyl)-2-(piperidin-4-yl)benzo[d]thiazole-6-sulfonamide

To a stirred solution of tert-butyl 4-(6-(N-(4,4-difluorocyclohexyl)sulfamoyl)benzo[d]thiazol-2-yl)piperidine-1-carboxylate (1.1 g, 2.13 mmol) in DCM (20 mL) at 0° C. was added HCl (4.0 M in 1,4-dioxane; 1.067 ml, 4.27 mmol). The reaction mixture was stirred at r.t. for 16 h, then concentrated under reduced pressure. The residue was triturated with methyl tert-butyl ether and the solid filtered off and dried to afford the title compound (800 mg, 56%) as a brown solid. A portion of the product was further purified by preparative HPLC using ammonium bicarbonate as buffer to afford N-(4,4-difluorocyclohexyl)-2-(piperidin-4-yl)benzo[d]thiazole-6-sulfonamide as an off-white solid. ¹H NMR δ_{H} (400 MHz, DMSO-d₆) 8.60 (1H, d, J 1.6), 8.09-8.17 (1H, m), 7.87-7.94 (2H, m), 3.21-3.34 (1H, m), 3.01-3.08 (2H, m), 2.65-2.70 (3H, m), 2.00-2.09 (2H, m), 1.57-1.98 (8H, m), 1.38-1.49 (2H, m).

### Example 292: N-(4,4-difluorocyclohexyl)-2-(1-(2-hydroxyethyl)piperidin-4-yl)benzo[d]thiazole-6-sulfonamide

### Step 4 (Scheme 35)

To a stirred solution of N-(4,4-difluorocyclohexyl)-2-(piperidin-4-yl)benzo[d]thiazole-6-sulfonamide (200 mg, 0.48 mmol) in acetonitrile (20 mL) was added 2-iodoethan-1-ol (99 mg, 0.58 mmol) and potassium carbonate (200 mg, 1.44 mmol). The reaction mixture was stirred at 80° C. for 16 h, then diluted with water and extracted with DCM (2×100 mL). The combined organic extracts were washed with water and brine, dried over anhydrous sodium sulfate and concentrated under reduced pressure. The residue was purified by preparative HPLC using ammonium bicarbonate as buffer to afford N-(4,4-difluorocyclohexyl)-2-(1-(2-hydroxyethyl)piperidin-4-yl)benzo[d]thiazole-6-sulfonamide (66 mg, 29%) as an off-white solid. ¹H NMR δ_{H} (400 MHz, DMSO-d₆) 8.52-8.67 (1H, m), 8.07-8.21 (1H, m), 7.84-7.96 (2H, m), 4.31-4.46 (1H, m), 3.51-3.54 (2H, m), 3.07-3.28 (2H, m), 2.90-3.07 (2H, m), 2.34-2.47 (2H, m), 2.00-2.23 (4H, m), 1.70-1.97 (6H, m), 1.56-1.70 (2H, m), 1.31-1.51 (2H, m).

Biological Assays

Cell Culture

HEK 293 and MDCK cells (Public Health England, Cell Culture Collections) were maintained in Dulbecco's Modified Eagle's Medium (DMEM) supplemented with 10% (v/v) fetal bovine serum (FBS) (Seralab), 2 mM L-glutamine and 100 U/ml penicillin streptomycin cocktail (ThermoFisher); termed complete media. Clonal HEK 293 cell lines were maintained in complete media supplemented with 0.6 mg/mL G418 (Enzo Life Sciences). Mouse inner medullary collecting duct (m-IMCD3) cells [American Type Culture Collection (ATCC)] were maintained in media consisting of DMEM/Hams F-12 50/50 Mix (DMEM F12; Corning) supplemented with penicillin streptomycin cocktail (100 U/ml) and FBS (10% v/v) (DMEM F12-10% FBS).

Experiment 1: Measurement of Modulation of Enzyme Activity by PDE4 Long Form Activators of the Present Invention Using Full-Length Human PDE4 Isoforms: Long Forms PDE4D5, PDE4C3, PDE4B1 and Short Form PDE4B2

(Marchmont, R. J. and Houslay, M. D. Biochem. J. 187: 381-92, 1980)

Exogenous Expression of Long Form PDE4 Enzymes and Stable Cell Line Generation

For transient transfection of exogenous PDE4 long isoforms, HEK 293 cells were transfected with pcDNA3.1 or pDEST™ PDE4 expression vectors using Lipofectamine LTX/Plus reagent (Invitrogen) as outlined by the manufacturer.

Where stable cell lines were generated the clonal isolates were expanded to obtain cell lines that stably expressed the full-length human PDE4 long isoforms and the full length human PDE4B2 short isoform. These were called the HEK-PDE4D5, HEK-PDE4B1 and HEK-PDE4B2 cell lines, respectively.

Lysate Preparation (Using PDE4D5 as a Typical Example)

HEK-PDE4D5 cells were seeded out in 100 mm plates and incubated at 37° C. in an atmosphere of 5% CO₂, 95% air. Cell lysates were prepared using KHEM buffer [50 mM KCl, 10 mM EGTA, 50 mM HEPES (pH 7.2), 1.92 mM MgCl₂].

To prepare the cell lysates, the 100 mm plates containing the cells were placed on ice and washed with ice-cold PBS (phosphate buffered saline, pH 7.4). KHEM buffer (500 μl) was added to the cells. Cells were then scraped off the plate and triturated using a needle (BD Microlance™ 0.8, 40 mm). The lysed cells were then centrifuged at 2000 rpm for 10 minutes to remove cell debris and the supernatant (cell lysate containing recombinant PDE4D5) was transferred to a fresh tube and kept on ice.

Cytosol Fraction Preparation (Using PDE4D5 as a Typical Example)

The cell lysate containing recombinant PDE4D5 was transferred into a centrifuge tube and placed into an ultracentrifuge (BECKMAN COULTER) and spun at high speed (100,000 g) for 30 minutes at 4° C. The cytosol fraction was then collected and its protein amount determined using a BCA protein assay.

PDE Assay—(using PDE4D5 as a typical example)

PDE assays were performed in thin walled V-bottomed 96-well plates. The assays were performed at a final concentration of 10 mM Tris/5 mM MgCl₂ μlus PDE4D5 cell lysate cytosol fraction, containing over-expressed PDE4D5, with and without test compound. The lysate/compound mix were incubated together for 15 min at room temperature on an orbital shaker prior to addition of [³H] cAMP (final concentration 1 μM [³H] cAMP; Perkin Elmer) to a final volume of 50 μl per reaction. The reactions were then incubated for 10 minutes at 30° C., terminated by heating for 2 min at 95° C. and allowed to cool. Snake venom (12.5 μl of 1 mg/ml; Crotalus atrox, Sigma) was then added and the plates were agitated and incubated for a further 15 minutes at 30° C. Dowex ion exchange resin (Sigma, chloride form, 200-400 mesh; 200 μl; prepared as a 1:1 Dowex: water stock, thoroughly re-suspended and diluted 2:1 with ethanol) was then added to each well and the plates incubated for 15 min at room temperature on an orbital shaker ensuring sufficient agitation for resin suspension (550 RPM). The reaction mixture was then transferred to a 96 well filter plate (Millipore; 0.45 μM pore size) and filtered into a receiving 96-well plate to remove the dowex suspension. 30 μL of the filtered solution was then transferred to the wells of an Opti-plate (Perkin Elmer) 96 well assay plate and 120 μl of Microscint 40 scintillation fluid was added. The plate was then placed on an orbital shaker for 10 min at high speed (900 RPM) to mix the sample with scintillation fluid prior to quantitation using a plate based scintillation counter (Top-Count).

The % increase in counts in the presence of test compound at a particular concentration indicates the % increase in enzyme activity at that concentration.

Data are shown in FIG. 1 and Tables 2 to 4.

Experiment 2: Reduction of Intracellular cAMP Levels in MDCK Cells by PDE4 Long Form Activators

MDCK cells were seeded at 100,000 cells per well, and left to adhere overnight. The cells were then treated with the test compound for 40 minutes, prior to stimulation with forskolin (1 μM, Sigma) for 20 minutes. Media was aspirated, and hydrochloric acid (0.1M) was added to lyse the cells. The cAMP assay (Enzo Life Sciences) was performed according to the manufacturer's instructions.

PDE4 long form activators reduced intracellular cAMP levels in forskolin-stimulated MDCK cells.

Experiment 3: Inhibition of In Vitro Cyst Formation in MDCK Cells Treated with PDE4 Long Form Activators

In this study, the well-established three-dimensional (3D) MDCK cell model is used to investigate the effects of PDE4 long form activators on the formation of kidney cysts and evaluate their potential in the treatment of polycystic kidney diseases. 3D cysts are generated based on the method of Mao et al. (Mao, Z., Streets, A. J., Ong, A. C. M. Am. J. Physiol. Renal Physiol. 300 (6): F1375-F1384, 2011), with some modifications.

The assay is conducted in the wells of a 96-well plate culture dish using a total volume of 130 μl of collagen suspension in growth media for each matrix plug per individual well. Rat Collagen I (Fisher Scientific) is prepared on ice by neutralising with 1M NaOH and diluting with a 2×volume of DMEM-2% FBS. 30 μl of collagen/Media mix is added into the well of a 96 well plate and the collagen is set to a gel by incubating at 37° C. for at least 15 minutes. A second layer of 100 μl collagen/Matrigel suspension containing MDCK cells (1.0×10⁴ MDCK cells per well) is then layered over the first layer and the collagen/cell mix again set to a gel by incubating at 37° C. 100 μl DMEM growth media is added and cells are incubated at 37° C. for 24 hours. 24 hours after cell seeding, DMEM-2% FBS is added along with the test compound indicated in the presence of 300 nM prostaglandin E2 (PGE2) (Sigma Aldrich) in quadruplicate wells per condition. Media, together with the test compound and PGE2 are replenished every 3 days for 10-15 days.

After 10-15 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in MDCK cells. Data are shown in Table 6 as total cyst area (%), compared to 100% for (DMSO+PGE2) and 0% for DMSO control.

Experiment 4: Inhibition of In Vitro Cyst Formation in m-IMCD3 Cells Treated with PDE4 Long Form Activators

The mouse Inner Medullary Collecting Duct cell line (m-IMCD3) spontaneously forms cystic spheroids in 3D culture with a Type 1 collagen/Matrigel extracellular matrix. This process can be stimulated with agents which raise intracellular cAMP, such as PGE2, and is used as an in vitro model for the formation of cystic structures in the kidneys of patients with ADPKD.

Rat Collagen I (Fisher Scientific) is prepared on ice by neutralising with 1M NaOH and diluting with a 2×volume of DMEM/F12+10% FBS for coating plate (coating mix) and a 2.2 volume of DMEM/F12+10% FBS for cell plating (plating mix). This is mixed 1:1.1 with ice cold Matrigel (Corning) for coating plates (coating mix), and 1:0.95 with Matrigel for cell plating (plating mix).

The assay is conducted in the wells of a 96-well plate culture dish using a total volume of 130 μl of collagen/Matrigel/DMEM F12-10% FBS suspension in growth media for each matrix plug per individual well. Initially, 30 μl of collagen/Matrigel/DMEM F12-10% FBS (coating mix) is added into the well of a 96 well plate and the collagen is set to a gel by incubating at 37° C. for at least 15 minutes. A second layer of 100 μl collagen/Matrigel suspension (plating mix) containing m-IMCD3 cells (2.75×10⁶ m-IMCD3 cells per 96-well plate) is layered over the coating mix and the collagen/Matrigel/cell mix again set to a gel by incubating at 37° C. Cell cultures are maintained at 37° C. in an atmosphere of 5% CO₂, 95% air.

Between 18 and 24h after plating, test compound(s) as DMSO stock solutions [0.1% (v/v) final DMSO concentration] and PGE2 (100 nM final concentration) in DMEM F12-10% FBS are added in quadruplicate wells per condition. Media, togetherwith the test compound and PGE2 are replenished after 2 or 3 days. After 6 days of culture z stack images of the wells are captured using the Nikon Eclipse Ti2-E microscope. Nikon General Analysis software is used to measure the following parameters in each well: the mean cyst area, the number of cysts, and the total cyst area.

PDE4 long form activators inhibited in vitro cyst formation in m-IMCD3 cells. Data are shown in FIG. 2 and Table 5 as mean cyst area (%), compared to 100% for (DMSO+PGE2) and 0% for DMSO control.

Experiment 4a: Reduction of cAMP Levels in m-IMCD3 Cell Culture Treated with PDE4 Long Form Activators Activation of intracellular PDE4 long forms leads to a reduction of both intracellular cAMP and externalised cAMP (Omar et al., PNAS 116: 13320-13329, 2019). This reduction may be measured in the cell culture media supernatant of 3D cystic cell cultures, such as in the m-IMCD3 cell cyst suppression assay described in Experiment 4.

Following the completion of the m-IMCD3 cell cyst suppression assay, the cAMP levels in the assay media from individual assay wells was measured using a cAMP ELISA kit (Enzo Life Sciences) according to the manufacturer's instructions.

Treatment with PDE4 long form activators reduced cAMP levels in PGE2-stimulated m-IMCD3 cell culture. Data are shown in Table 7.

Experiment 5: Inhibition of Proliferation of LNCaP Human Prostate Cancer Cells

In this study, the potential utility of PDE4 long form activators in the treatment of prostate cancer is studied using the LNCaP human prostate cancer cell line. The experiments are carried out according to the method described by Henderson et al. (Henderson, D. J. P., Byrne, A., Dulla, K., Jenster, G., Hoffmann, R., Baillie, G. S., Houslay, M. D. Br. J. Cancer 110: 1278-1287, 2014).

LNCaP Cell Culture Androgen-sensitive (AS) LNCaP cells are maintained in RPM11640 supplemented with 10% FBS (Seralabs), 2 mM L-glutamine and 1,000U penicillin-streptomycin. LNCaP androgen-insensitive (AI) cells are generated by culturing the LNCaP-AS cells in RPM11640 supplemented with 10% charcoal stripped FBS, 2 mM L-glutamine and 1,000U penicillin-streptomycin for a minimum of four weeks. All tissue culture reagents are from Life Technologies.

Xcelligence (Roche) Proliferation Assay

Cell proliferation is measured as a function of changing electrical impedance. Values are represented by cell index number, a dimensionless unit of measurement representing the cell status, which increases as cells adhere to 96-well electrode plates and divide.

LNCaP AI/AS cells are plated at a density of 25,000 cells per well in a 96-well electrode plate (in triplicate), in the presence/absence of various concentrations of test compound.

Cell indices are measured every 10 minutes for up to 100 hours, analysed using RTCA software and normalised to the cell index of vehicle-treated cells (n=3).

PDE4 long form activators inhibited the proliferation of AS and AI LNCaP human prostate cancer cells.

Experiment 6: In Vivo Preclinical Model of Hyperparathyroidism: Inhibition of PTH-Induced cAMP Elevation in Urine in the Anaesthetised Rat

Within the kidney, the binding of parathyroid hormone (PTH) to PTH receptors results in the Gₐₛ-mediated elevation of intracellular cAMP. This increase of intracellular cAMP results in extrusion of cAMP to the urine (Yates et al., J Clin Invest 81: 932-938, 1988). This experiment is based upon a modified Ellsworth-Howard assay (Kruse, K. and Kracht, U., European Journal of Pediatrics 146: 373-377, 1987) and conducted in anaesthetized rats. In this experiment, rats were anaesthetized using isoflurane and catheterised to allow the collection of urine from the bladder. After an initial stabilization period, test compounds were administered by i.v. infusion (modelled to steady state) from time 0-120 min. PTH challenge infusion (33 ug/kg/hour) was started after 60 min of test compound infusion and sustained for one hour (60-120 min). Urine collection was conducted in 30-minute periods. Urine cAMP levels were assessed by ELISA (R&D systems). Urine samples were prepared for analysis as per the manufacturer's instructions. A standard curve for cAMP was assayed for each experiment and samples were assessed using a standard dilution range of 1:2, 1:4, 1:8 and 1:16, ensuring that the resulting data remained on the linear portion of the standard curve. Control animals treated with vehicle alone (no PTH) showed no increase from baseline in urinary cAMP concentrations over the course of the experiment. Urine cAMP concentration remained below 50,000 pmol/mL. Control animals treated with PTH infusion plus vehicle (PTH challenge) showed an increase from baseline in cAMP concentration in urine collected from 90 to 120 m(D.

Treatment with PDE4 long form activators suppressed the elevation in cAMP concentration in response to PTH challenge in urine collected from 90 to 120 min. Data are shown in FIG. 3.

TABLE 1
Small molecule PDE4 long form activators (Examples 1 to 292), according to the present invention

| | | Molecular | |
|---|---|---|---|
| | | weight | |
| Compound | Chemical name | (Daltons) | Chemical Structure |
| Example 1 | N-(4-chlorobenzyl)-2- (2-methylpyridin-3- yl)benzo[d]thiazole-6- carboxamide | 393.9 | 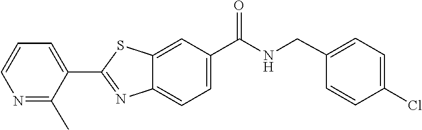 [SMILES: Cc1ncccc1-c1nc2ccc(C(=O)NCc3ccc(Cl)cc3)cc2s1] |
| Example 2 | N-(4-fluorobenzyl)-2- (2-methylpyridin-3- yl)benzo[d]thiazole- 6-carboxamide | 377.4 | 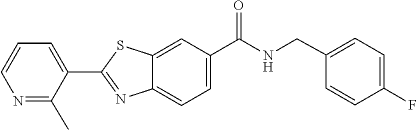 [SMILES: Cc1ncccc1-c1nc2ccc(C(=O)NCc3ccc(F)cc3)cc2s1] |
| Example 3 | N-(4-methoxybenzyl)- 2-(2-methylpyridin-3- yl)benzo[d]thiazole- 6-carboxamide | 389.5 | 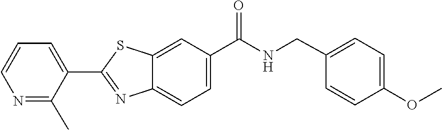 [SMILES: COc1ccc(CNC(=O)c2ccc3nc(-c4cccnc4C)sc3c2)cc1] |
| Example 4 | N-(3-fluorobenzyl)-2- (2-methylpyridin-3-yl)- benzo[d]thiazole-6- carboxamide | 377.4 | 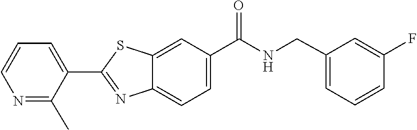 [SMILES: Cc1ncccc1-c1nc2ccc(C(=O)NCc3cccc(F)c3)cc2s1] |
| Example 5 | (S)-N-(2,3-dihydro-1H- inden-1-yl)-2-(2-methyl- pyridin-3-yl)benzo[d]- thiazole-6-carboxamide | 385.5 | 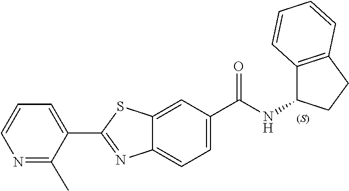 |
| Example 6 | (S)-2-(2-methylpyridin- 3-yl)-N-(1,2,3,4-tetra- hydronaphthalen-1- yl)benzo[d]thiazole-6- carboxamide | 399.5 | 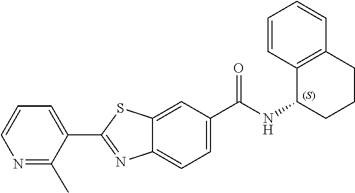 |
| Example 7 | (S)-N-(chroman-4-yl)- 2-(2-methylpyridin-3- yl)benzo[d]thiazole- 6-carboxamide | 401.5 | 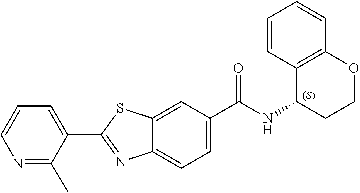 |
| Example 8 | (S)-2-(pyridin-3-yl)- N-(1,2,3,4-tetrahydro- naphthalen-1-yl)- benzo[d]thiazole-6- carboxamide | 385.5 | 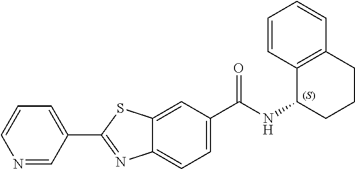 |
| Example 9 | (S)-N-(chroman-4-yl)- 2-(pyridin-3-yl)benzo- [d]thiazole-6-carbox- amide | 387.5 | 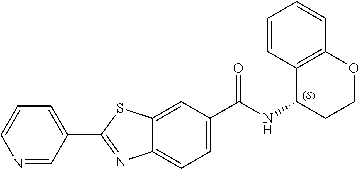 |
| Example 10 | N-benzyl-2-(6-methyl- pyridin-3-yl)benzo[d]- thiazole-6-carboxamide | 359.5 | 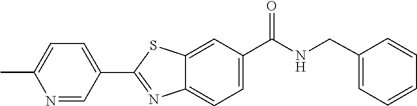 [SMILES: Cc1ccc(-c2nc3ccc(C(=O)NCc4ccccc4)cc3s2)cn1] |
| Example 11 | N-(4-chlorobenzyl)-2- (6-methylpyridin-3- yl)benzo[d]thiazole- 6-carboxamide | 393.9 | 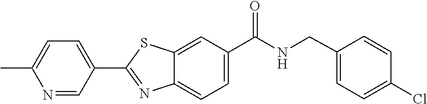 [SMILES: Cc1ccc(-c2nc3ccc(C(=O)NCc4ccc(Cl)cc4)cc3s2)cn1] |
| Example 12 | N-(4-fluorobenzyl)-2- (6-methylpyridin-3- yl)benzo[d]thiazole- 6-carboxamide | 377.4 | 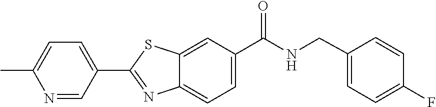 [SMILES: Cc1ccc(-c2nc3ccc(C(=O)NCc4ccc(F)cc4)cc3s2)cn1] |
| Example 13 | N-(4-methoxybenzyl)- 2-(6-methylpyridin-3- yl)benzo[d]thiazole- 6-carboxamide | 389.5 | 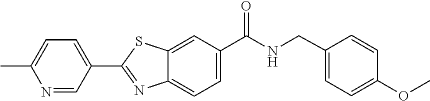 |
| Example 14 | N-(3-chlorobenzyl)-2- (6-methylpyridin-3- yl)benzo[d]thiazole- 6-carboxamide | 393.9 | 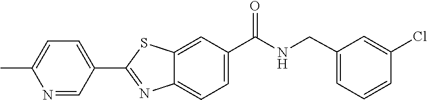 [SMILES: Cc1ccc(-c2nc3ccc(C(=O)NCc4cccc(Cl)c4)cc3s2)cn1] |
| Example 15 | N-(3-fluorobenzyl)-2- (6-methylpyridin-3- yl)benzo[d]thiazole- 6-carboxamide | 377.4 | 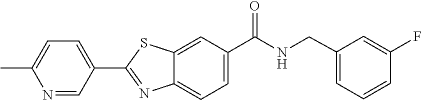 [SMILES: Cc1ccc(-c2nc3ccc(C(=O)NCc4cccc(F)c4)cc3s2)cn1] |
| Example 16 | N-(3-methoxybenzyl)- 2-(6-methylpyridin-3- yl)benzo[d]thiazole- 6-carboxamide | 389.5 | 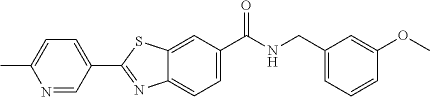 |
| Example 17 | (S)-N-(2,3-dihydro- 1H-inden-1-yl)-2-(6- methylpyridin-3-yl)- benzo[d]thiazole-6- carboxamide | 385.5 | 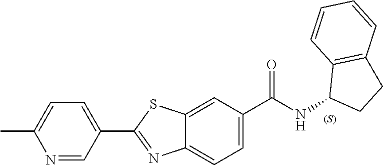 |
| Example 18 | (R)-N-(2,3-dihydro- 1H-inden-1-yl)-2- (6-methylpyridin-3- yl)benzo[d]thiazole- 6-carboxamide | 385.5 | 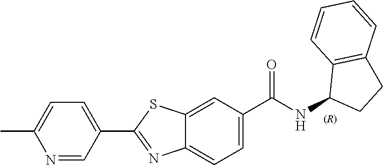 |
| Example 19 | N-((1S,2R)-2-hydroxy- 2,3-dihydro-1H-inden- 1-yl)-2-(6-methyl- pyridin-3-yl)benzo[d]- thiazole-6-carboxamide | 401.5 | 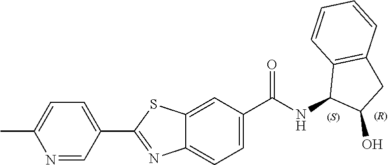 |
| Example 20 | N-((1R,2S)-2-hydroxy- 2,3-dihydro-1H-inden- 1-yl)-2-(6-methyl- pyridin-3-yl)benzo[d]- thiazole-6-carboxamide | 401.5 | 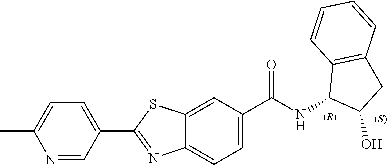 |
| Example 21 | N-((1R,2R)-2-hydroxy- 2,3-dihydro-1H-inden- 1-yl)-2-(6-methylpyr- idin-3-yl)benzo[d]thia- zole-6-carboxamide | 401.5 | 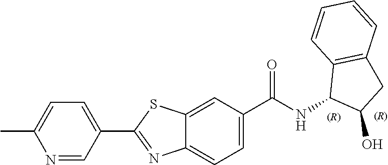 |
| Example 22 | N-cyclopentyl-2-(6- methylpyridin-3-yl)- benzo[d]thiazole-6- carboxamide | 337.4 | 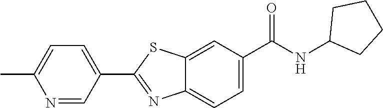 [SMILES: Cc1ccc(-c2nc3ccc(C(=O)NC4CCCC4)cc3s2)cn1] |
| Example 23 | (S)-2-(6-methyl- pyridin-3-yl)-N- (1,2,3,4-tetrahydro- naphthalen-1-yl)- benzo[d]thiazole-6- carboxamide | 399.5 | 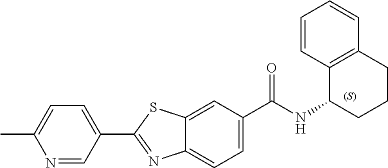 |
| Example 24 | (S)-N-(chroman-4- yl)-2-(6-methyl- pyridin-3-yl)benzo- [d]thiazole-6-carbox- amide | 401.5 | 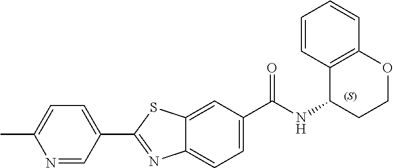 |
| Example 25 | (S)-N-(chroman-4-yl)- 2-(6-(trifluorometh- yl)pyridin-3-yl)benzo- [d]thiazole-6-carbox- amide | 455.5 | 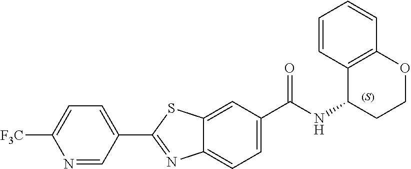 |
| Example 26 | (S)-N-(chroman-4- yl)-2-(2,6-dimethyl- pyridin-3-yl)benzo- [d]thiazole-6-carbox- amide | 415.5 | 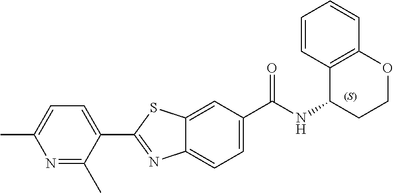 |
| Example 27 | (S)-N-(chroman-4- yl)-2-(6-cyclopropyl- pyridin-3-yl)benzo- [d]thiazole-6-carbox- amide | 427.5 | 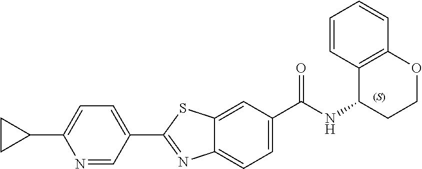 |
| Example 28 | (S)-N-(chroman-4- yl)-2-(6-isopropyl- pyridin-3-yl)benzo- [d]thiazole-6-carbox- amide | 429.5 | 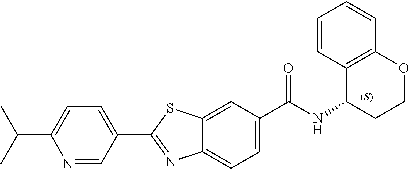 |
| Example 29 | (S)-N-(chroman-4- yl)-2-(6-ethylpyridin- 3-yl)benzo[d]thiazole- 6-carboxamide | 415.5 | 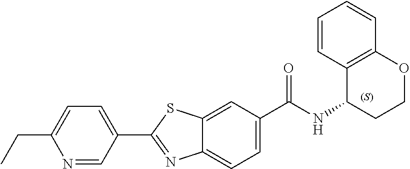 |
| Example 30 | N-benzyl-2-(5-meth- ylpyridin-3-yl)benzo- [d]thiazole-6-carbox- amide | 359.5 | 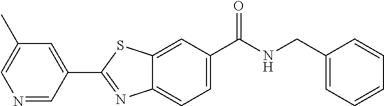 [SMILES: Cc1cncc(-c2nc3ccc(C(=O)NCc4ccccc4)cc3s2)c1] |
| Example 31 | N-(4-chlorobenzyl)- 2-(5-methylpyridin- 3-yl)benzo[d]thiazole- 6-carboxamide | 393.9 | 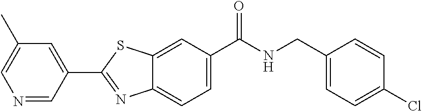 [SMILES: Cc1cncc(-c2nc3ccc(C(=O)NCc4ccc(Cl)cc4)cc3s2)c1] |
| Example 32 | N-(4-fluorobenzyl)- 2-(5-methylpyridin- 3-yl)benzo[d]thiazole- 6-carboxamide | 377.4 | 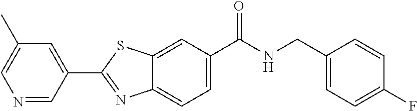 [SMILES: Cc1cncc(-c2nc3ccc(C(=O)NCc4ccc(F)cc4)cc3s2)c1] |
| Example 33 | N-(4-methoxybenzyl)- 2-(5-methylpyridin-3- yl)benzo[d]thiazole- 6-carboxamide | 389.5 | 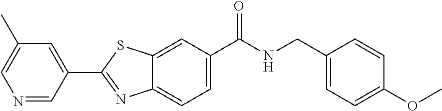 [SMILES: COc1ccc(CNC(=O)c2ccc3nc(-c4cncc(C)c4)sc3c2)cc1] |
| Example 34 | N-(3-chlorobenzyl)- 2-(5-methylpyridin- 3-yl)benzo[d]thiazole- 6-carboxamide | 393.9 | 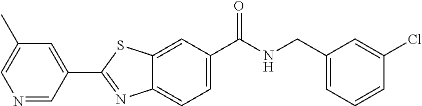 [SMILES: Cc1cncc(-c2nc3ccc(C(=O)NCc4cccc(Cl)c4)cc3s2)c1] |
| Example 35 | N-(3-fluorobenzyl)- 2-(5-methylpyridin- 3-yl)benzo[d]thiazole- 6-carboxamide | 377.4 | 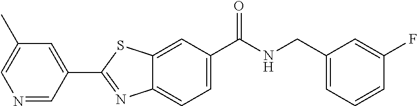 [SMILES: Cc1cncc(-c2nc3ccc(C(=O)NCc4cccc(F)c4)cc3s2)c1] |
| Example 36 | N-(3-methoxybenzyl)- 2-(5-methylpyridin-3- yl)benzo[d]thiazole- 6-carboxamide | 389.5 | 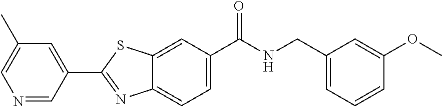 [SMILES: COc1cccc(CNC(=O)c2ccc3nc(-c4cncc(C)c4)sc3c2)c1] |
| Example 37 | (S)-N-(2,3-dihydro- 1H-inden-1-yl)-2-(5- methylpyridin-3-yl)- benzo[d]thiazole-6- carboxamide | 385.5 | 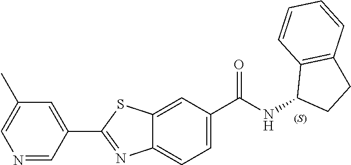 |
| Example 38 | (R)-N-(2,3-dihydro- 1H-inden-1-yl)-2- (5-methylpyridin-3- yl)benzo[d]thiazole- 6-carboxamide | 385.5 | 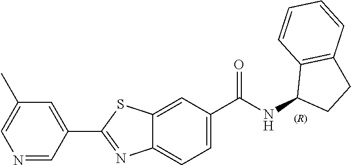 |
| Example 39 | N-((1R,2S)-2- hydroxy-2,3-dihydro- 1H-inden-1-yl)-2-(5- methylpyridin-3-yl)- benzo[d]thiazole-6- carboxamide | 401.5 | 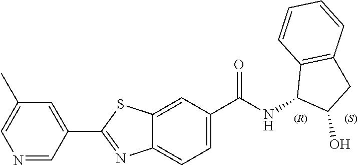 |
| Example 40 | N-((1S,2R)-2- hydroxy-2,3-dihydro- 1H-inden-1-yl)-2- (5-methylpyridin-3- yl)benzo[d]thiazole- 6-carboxamide | 401.5 | 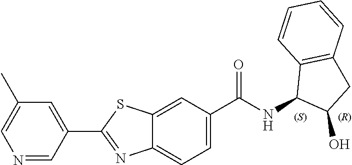 |
| Example 41 | N-((1S,2S)-2- hydroxy-2,3-dihydro- 1H-inden-1-yl)-2-(5- methylpyridin-3- yl)benzo[d]thiazole- 6-carboxamide | 401.5 | 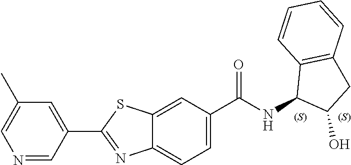 |
| Example 42 | N-((1R,2R)-2- hydroxy-2,3-dihydro- 1H-inden-1-yl)-2- (5-methylpyridin-3- yl)benzo[d]thiazole- 6-carboxamide | 401.5 | 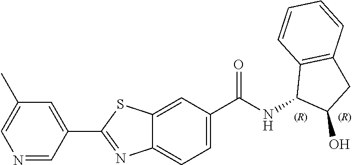 |
| Example 43 | N-cyclopentyl-2- (5-methylpyridin-3- yl)benzo[d]thiazole- 6-carboxamide | 337.4 | 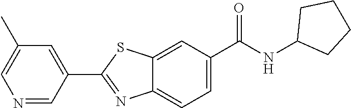 [SMILES: Cc1cncc(-c2nc3ccc(C(=O)NC4CCCC4)cc3s2)c1] |
| Example 44 | (S)-2-(5-methyl- pyridin-3-yl)-N- (1,2,3,4-tetrahydro- naphthalen-1-yl)- benzo[d]thiazole- 6-carboxamide | 399.5 | 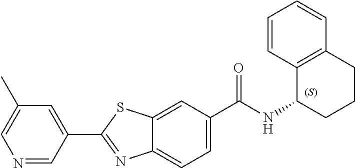 |
| Example 45 | (S)-N-(chroman-4-yl)- 2-(5-methylpyridin-3- yl)benzo[d]thiazole-6- carboxamide | 401.5 | 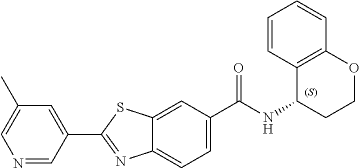 |
| Example 46 | 2-(5-methylpyridin- 3-yl)-N-(tetrahydro- 2H-pyran-4-yl)- benzo[d]thiazole-6- carboxamide | 353.4 | 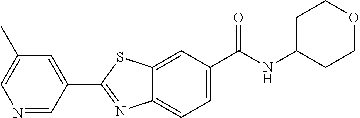 [SMILES: Cc1cncc(-c2nc3ccc(C(=O)NC4CCOCC4)cc3s2)c1] |
| Example 47 | (S)-N-(chroman-4- yl)-2-(5-(trifluoro- methyl)pyridin-3- yl)benzo[d]thiazole- 6-carboxamide | 455.5 | 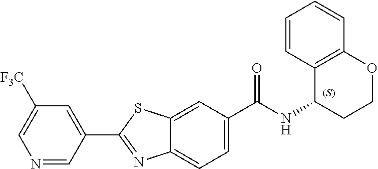 |
| Example 48 | (S)-N-(chroman-4- yl)-2-(5-cyclopropyl- pyridin-3-yl)benzo- [d]thiazole-6-carbox- amide | 427.5 | 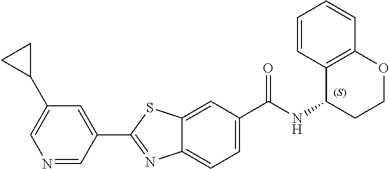 |
| Example 49 | (S)-N-(chroman-4- yl)-2-(5-isopropyl- pyridin-3-yl)benzo- [d]thiazole-6-carbox- amide | 429.5 | 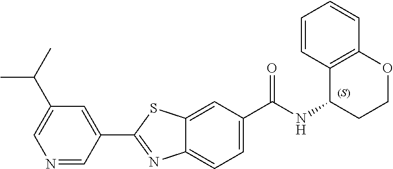 |
| Example 50 | (S)-2-(4-methyl- pyridin-3-yl)-N- (1,2,3,4-tetrahydro- naphthalen-1-yl)- benzo[d]thiazole- 6-carboxamide | 399.5 | 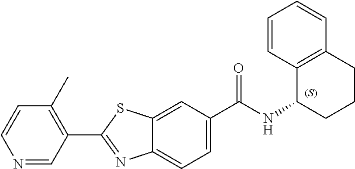 |
| Example 51 | (S)-N-(chroman-4- yl)-2-(4-methyl- pyridin-3-yl)benzo- [d]thiazole-6-carbox- amide | 401.5 | 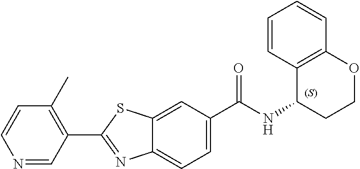 |
| Example 52 | (S)-N-(chroman-4- yl)-2-(2,4-dimethyl- pyridin-3-yl)benzo[d]- thiazole-6-carboxamide | 415.5 | 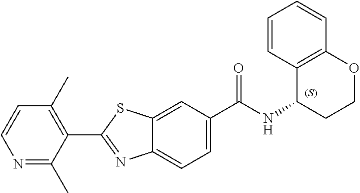 |
| Example 53 | (S)-2-(5-chloropyridin- 3-yl)-N-(chroman-4- yl)benzo[d]thiazole-6- carboxamide | 421.9 | 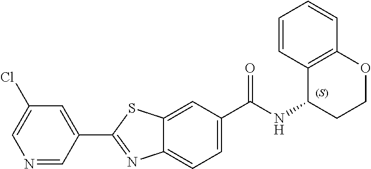 |
| Example 54 | (S)-N-(chroman-4-yl)- 2-(pyridin-4-yl)benzo- [d]thiazole-6-carbox- amide | 387.5 | 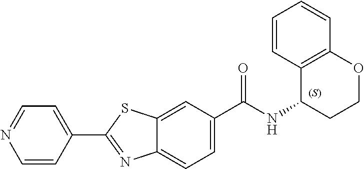 |
| Example 55 | (S)-N-(chroman-4-yl)- 2-(3-methylpyridin-4- yl)benzo[d]thiazole- 6-carboxamide | 401.5 | 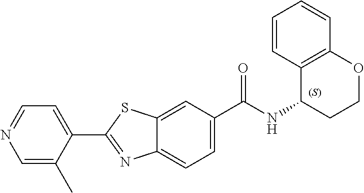 |
| Example 56 | (S)-N-(chroman-4-yl)- 2-(2-methylpyridin-4- yl)benzo[d]thiazole- 6-carboxamide | 401.5 | 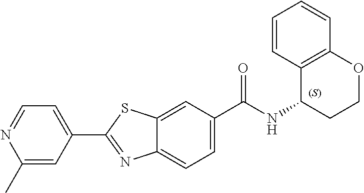 |
| Example 57 | (S)-N-(chroman-4- yl)-2-(1-methyl-1H- pyrazol-4-yl)benzo- [d]thiazole-6-carbox- amide | 390.5 | 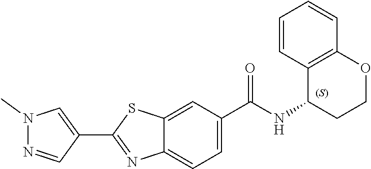 |
| Example 58 | (S)-N-(chroman-4- yl)-2-(1,4-dimethyl- 1H-pyrazol-5-yl)- benzo[d]thiazole-6- carboxamide | 404.5 | 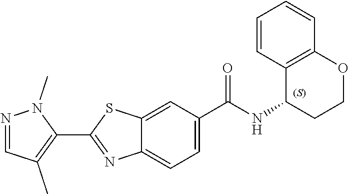 |
| Example 59 | (S)-N-(chroman-4- yl)-2-(1,3,5-trimethyl- 1H-pyrazol-4-yl)- benzo[d]thiazole-6- carboxamide | 418.5 | 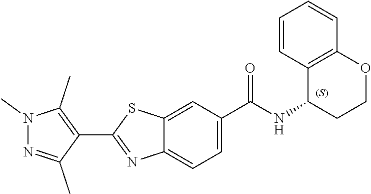 |
| Example 60 | (S)-N-(chroman-4-yl)- 2-(1,5-dimethyl-1H- pyrazol-4-yl)benzo- [d]thiazole-6-carbox- amide | 404.5 | 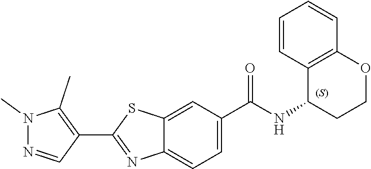 |
| Example 61 | (S)-N-(chroman-4-yl)- 2-(1,3-dimethyl-1H- pyrazol-4-yl)benzo- [d]thiazole-6-carbox- amide | 404.5 | 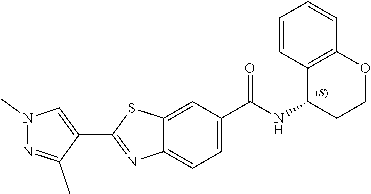 |
| Example 62 | (S)-N-(chroman-4-yl)- 2-(5-(hydroxymethyl)- pyridin-3-yl)benzo- [d]thiazole-6-carbox- amide | 417.5 | 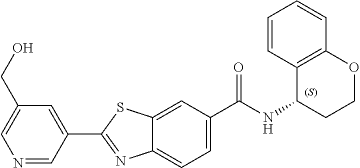 |
| Example 63 | (S)-N-(chroman-4-yl)- 2-(5-(difluoromethyl)- pyridin-3-yl)benzo[d]- thiazole-6-carboxamide | 437.5 | 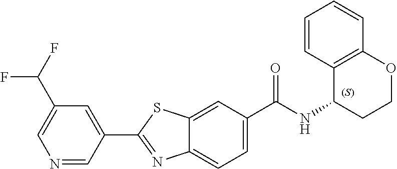 |
| Example 64 | (S)-N-(chroman-4-yl)- 2-(6-(difluoromethyl)- pyridin-3-yl)benzo[d]- thiazole-6-carboxamide | 437.5 | 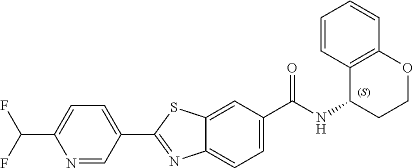 |
| Example 65 | (S)-N-(chroman-4-yl)- 2-(2-methylpyridin-3- yl)benzo[d]thiazole- 5-carboxamide | 401.5 | 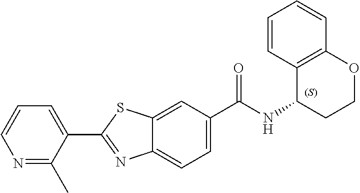 |
| Example 66 | (S)-N-(chroman-4-yl)- 2-(4-methylpyridin-3- yl)benzo[d]thiazole- 5-carboxamide | 401.5 | 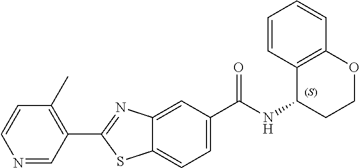 |
| Example 67 | (S)-N-(chroman-4-yl)- 2-(pyridin-3-yl)benzo- [d]thiazole-5-carbox- amide | 387.5 | 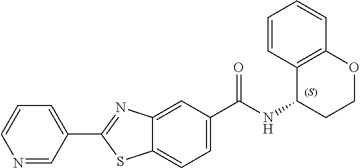 |
| Example 68 | (S)-N-(chroman-4-yl)- 2-(4-hydroxypiperidin- 1-yl)benzo[d]thiazole- 6-carboxamide | 409.5 | 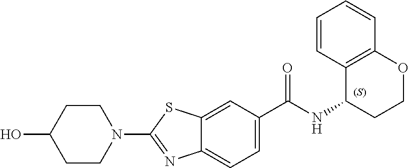 |
| Example 69 | (S)-N-(chroman-4-yl)- 2-morpholinobenzo- [d]thiazole-6-carbox- amide | 395.5 | 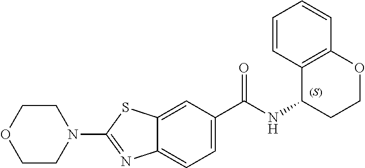 |
| Example 70 | (S)-N-(chroman-4-yl)- 2-(4-methoxypiperidin- 1-yl)benzo[d]thiazole- 6-carboxamide | 423.5 | 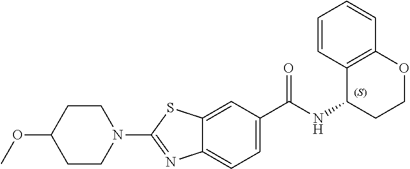 |
| Example 71 | (S)-N-(chroman-4-yl)- 2-(4-methylpiperazin- 1-yl)benzo[d]thiazole- 6-carboxamide | 408.5 | 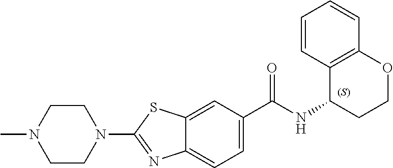 |
| Example 72 | (S)-N-(chroman-4-yl)- 2-(4-ethylpiperazin-1- yl)benzo[d]thiazole- 6-carboxamide | 422.6 | 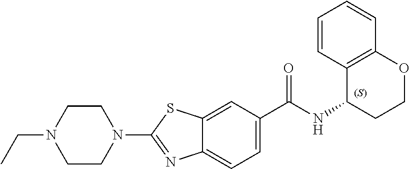 |
| Example 73 | (R)-N-(chroman-4-yl)- 2-(4-ethylpiperazin-1- yl)benzo[d]thiazole- 6-carboxamide | 422.6 | 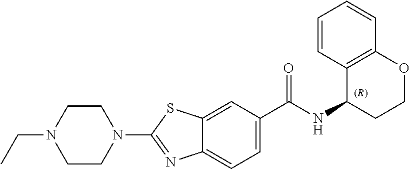 |
| Example 74 | (S)-N-(chroman-4-yl)- 2-(4-isopropylpiper- azin-1-yl)benzo[d]- thiazole-6-carbox- amide | 436.6 | 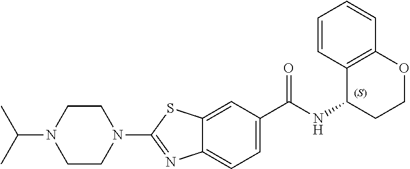 |
| Example 75 | (S)-2-(4-(tert-butyl)- piperazin-1-yl)-N- (chroman-4-yl)benzo- [d]thiazole-6-carbox- amide | 450.6 | 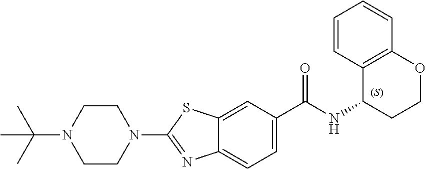 |
| Example 76 | (S)-N-(chroman-4- yl)-2-(piperidin-1- yl)benzo[d]thiazole- 6-carboxamide | 393.5 | 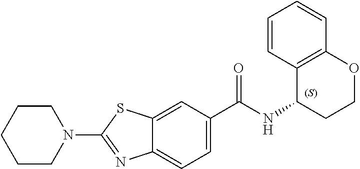 |
| Example 77 | 2-((1S,4S)-2,5- diazabicyclo[2.2.1]- heptan-2-yl)-N-((S)- chroman-4-yl)benzo- [d]thiazole-6-carbox- amide | 406.5 | 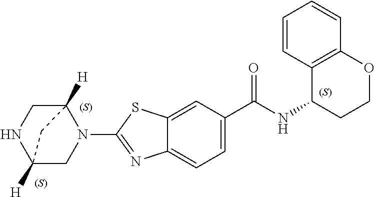 |
| Example 78 | 2-((1R,4R)-2,5- diazabicyclo[2.2.1]- heptan-2-yl)-N-((S)- chroman-4-yl)benzo- [d]thiazole-6-carbox- amide | 406.5 | 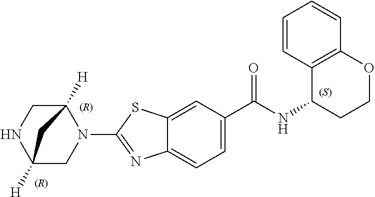 |
| Example 79 | 2-(2,5-diazabicyclo- [2.2.2]octan-2-yl)- N-((S)-chroman-4- yl)benzo[d]thiazole- 6-carboxamide | 420.5 | 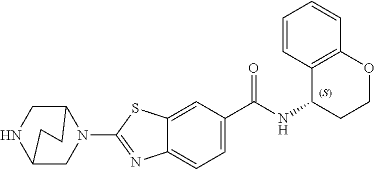 |
| Example 80 | 2-(4-ethylpiperazin- 1-yl)-N-(4-fluoro- benzyl)benzo[d]- thiazole-6-carbox- amide | 398.5 | 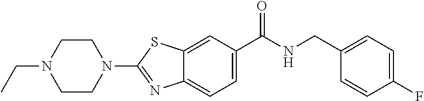 [SMILES: CCN1CCN(c2nc3ccc(C(=O)NCc4ccc(F)cc4)cc3s2)CC1] |
| Example 81 | N-cyclopentyl-2-(4- ethylpiperazin-1- yl)benzo[d]thiazole- 6-carboxamide | 358.5 | 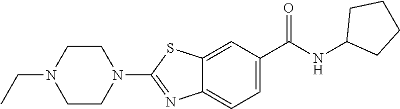 [SMILES: CCN1CCN(c2nc3ccc(C(=O)NC4CCCC4)cc3s2)CC1] |
| Example 82 | 2-(4-ethylpiperazin- 1-yl)-N-isopropyl- benzo[d]thiazole-6- carboxamide | 332.5 | 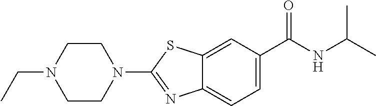 [SMILES: CCN1CCN(c2nc3ccc(C(=O)NC(C)C)cc3s2)CC1] |
| Example 83 | (S)-N-(chroman-4- yl)-2-(piperazin-1-yl)- benzo[d]thiazole-6- carboxamide | 394.5 | 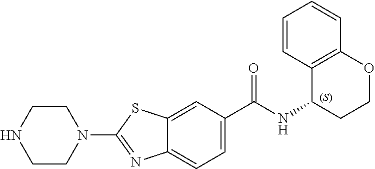 |
| Example 84 | (R)-N-(chroman-4- yl)-2-(piperazin-1-yl)- benzo[d]thiazole-6- carboxamide | 394.5 | 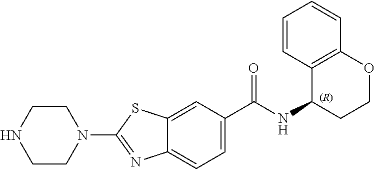 |
| Example 85 | (S)-N-(8-fluorochro- man-4-yl)-2-(piperazin- 1-yl)benzo[d]thiazole- 6-carboxamide | 412.5 | 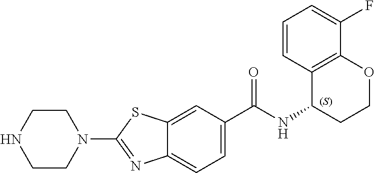 |
| Example 86 | (R)-N-(8-fluorochro- man-4-yl)-2-(piperazin- 1-yl)benzo[d]thiazole- 6-carboxamide | 412.5 | 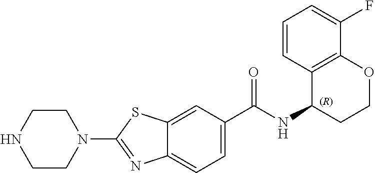 |
| Example 87 | (S)-N-(7-fluorochro- man-4-yl)-2-(piperazin- 1-yl)benzo[d]thiazole- 6-carboxamide | 412.5 | 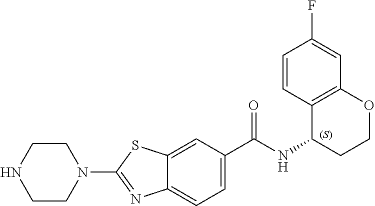 |
| Example 88 | (R)-N-(7-fluorochro- man-4-yl)-2-(piperazin- 1-yl)benzo[d]thiazole- 6-carboxamide | 412.5 | 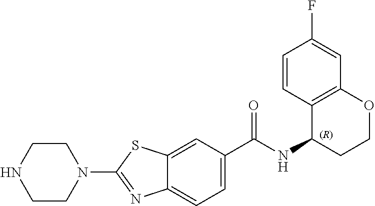 |
| Example 89 | (S)-N-(6-fluorochro- man-4-yl)-2-(piperazin- 1-yl)benzo[d]thiazole- 6-carboxamide | 412.5 | 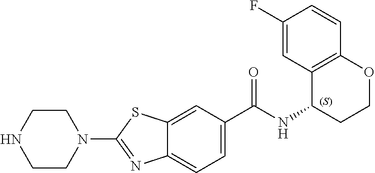 |
| Example 90 | (S)-N-(5-fluorochro- man-4-yl)-2-(piperazin- 1-yl)benzo[d]thiazole- 6-carboxamide | 412.5 | 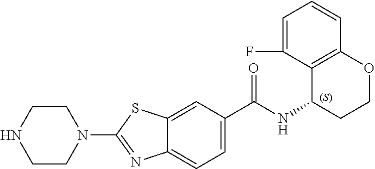 |
| Example 91 | (R)-N-(5-fluorochro- man-4-yl)-2-(piperazin- 1-yl)benzo[d]thiazole- 6-carboxamide | 412.5 | 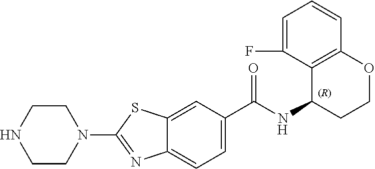 |
| Example 92 | (S)-N-(7-methoxy- chroman-4-yl)-2- (piperazin-1-yl)- benzo[d]thiazole- 6-carboxamide | 424.5 | 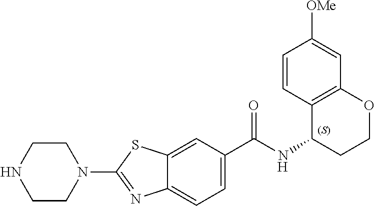 |
| Example 93 | (R)-N-(7-methoxy- chroman-4-yl)-2- (piperazin-1-yl)- benzo[d]thiazole- 6-carboxamide | 424.5 | 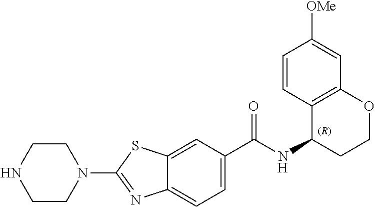 |
| Example 94 | (S)-N-(6-methoxy- chroman-4-yl)-2- (piperazin-1-yl)- benzo[d]thiazole- 6-carboxamide | 424.5 | 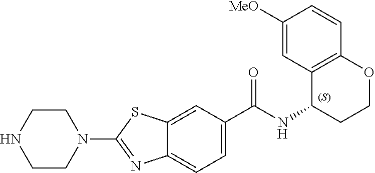 |
| Example 95 | (S)-N-(7-cyano- chroman-4-yl)-2- (piperazin-1-yl)- benzo[d]thiazole- 6-carboxamide | 419.5 | 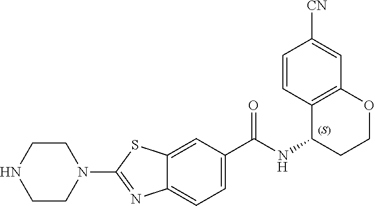 |
| Example 96 | (S)-N-(2,3-dihydro- 1H-inden-1-yl)-2- (piperazin-1-yl)- benzo[d]thiazole-6- carboxamide | 378.5 | 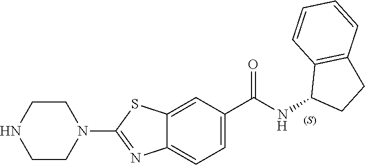 |
| Example 97 | (R)-N-(2,3-dihydro- 1H-inden-1-yl)-2- (piperazin-1-yl)benzo- [d]thiazole-6-carbox- amide | 378.5 | 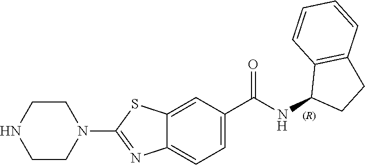 |
| Example 98 | (S)-N-(7-fluoro-2,3- dihydro-1H-inden-1- yl)-2-(piperazin-1- yl)benzo[d]thiazole- 6-carboxamide | 396.5 | 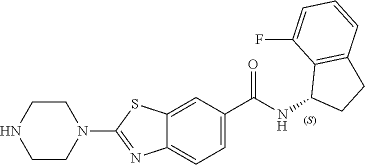 |
| Example 99 | (R)-N-(7-fluoro-2,3- dihydro-1H-inden-1- yl)-2-(piperazin-1- yl)benzo[d]thiazole- 6-carboxamide | 396.5 | 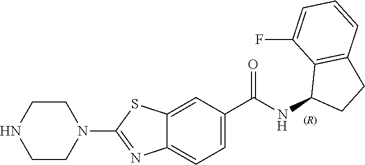 |
| Example 100 | (S)-N-(6-fluoro-2,3- dihydro-1H-inden-1- yl)-2-(piperazin-1- yl)benzo[d]thiazole- 6-carboxamide | 396.5 | 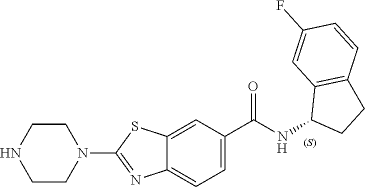 |
| Example 101 | (S)-N-(5-fluoro-2,3- dihydro-1H-inden-1- yl)-2-(piperazin-1- yl)benzo[d]thiazole- 6-carboxamide | 396.5 | 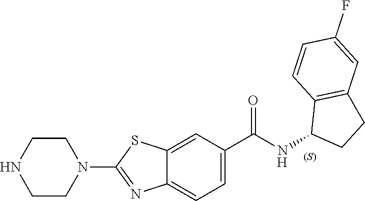 |
| Example 102 | (R)-N-(5-fluoro-2,3- dihydro-1H-inden- 1-yl)-2-(piperazin-1- yl)benzo[d]thiazole- 6-carboxamide | 396.5 | 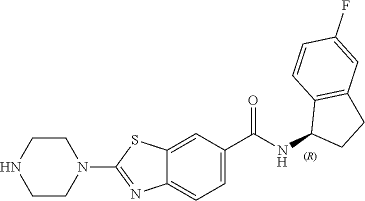 |
| Example 103 | (S)-N-(4-fluoro-2,3- dihydro-1H-inden- 1-yl)-2-(piperazin- 1-yl)benzo[d]thiazole- 6-carboxamide | 396.5 | 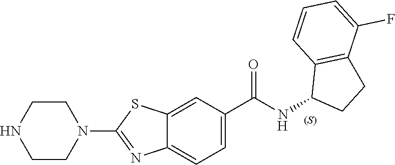 |
| Example 104 | (R)-N-(4-fluoro-2,3- dihydro-1H-inden- 1-yl)-2-(piperazin-1- yl)benzo[d]thiazole- 6-carboxamide | 396.5 | 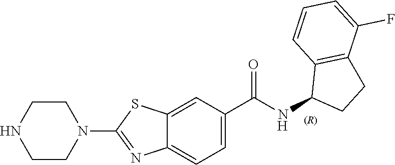 |
| Example 105 | (S)-N-(7-methoxy- 2,3-dihydro-1H-inden- 1-yl)-2-(piperazin-1- yl)benzo[d]thiazole- 6-carboxamide | 408.5 | 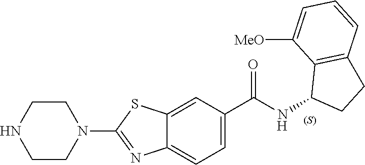 |
| Example 106 | (S)-N-(6-methoxy- 2,3-dihydro-1H-inden- 1-yl)-2-(piperazin-1- yl)benzo[d]thiazole- 6-carboxamide | 408.5 | 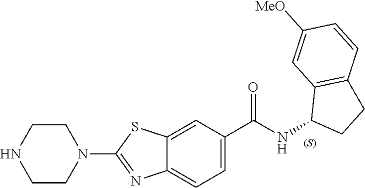 |
| Example 107 | (R)-N-(6-methoxy- 2,3-dihydro-1H-inden- 1-yl)-2-(piperazin-1- yl)benzo[d]thiazole- 6-carboxamide | 408.5 | 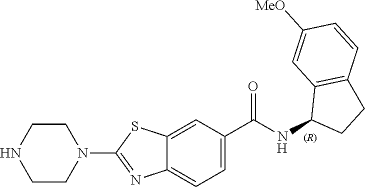 |
| Example 108 | (S)-N-(5-methoxy- 2,3-dihydro-1H-inden- 1-yl)-2-(piperazin-1- yl)benzo[d]thiazole- 6-carboxamide | 408.5 | 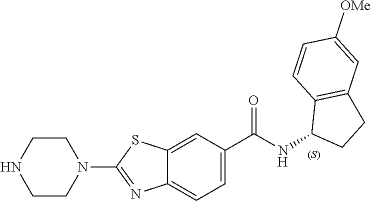 |
| Example 109 | (S)-N-(4-methoxy- 2,3-dihydro-1H-inden- 1-yl)-2-(piperazin-1- yl)benzo[d]thiazole- 6-carboxamide | 408.5 | 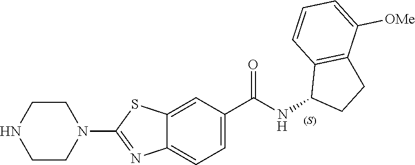 |
| Example 110 | (R)-N-(4-methoxy- 2,3-dihydro-1H-inden- 1-yl)-2-(piperazin-1- yl)benzo[d]thiazole- 6-carboxamide | 408.5 | 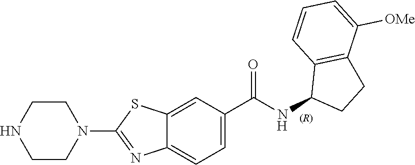 |
| Example 111 | (S)-N-(6-cyano-2,3- dihydro-1H-inden- 1-yl)-2-(piperazin-1- yl)benzo[d]thiazole- 6-carboxamide | 403.5 | 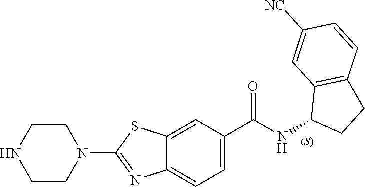 |
| Example 112 | (R)-N-(6-cyano-2,3- dihydro-1H-inden-1- yl)-2-(piperazin-1-yl)- benzo[d]thiazole-6- carboxamide | 403.5 | 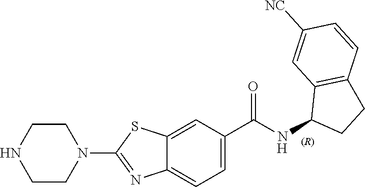 |
| Example 113 | (S)-N-(5-cyano-2,3- dihydro-1H-inden-1- yl)-2-(piperazin-1-yl)- benzo[d]thiazole-6- carboxamide | 403.5 | 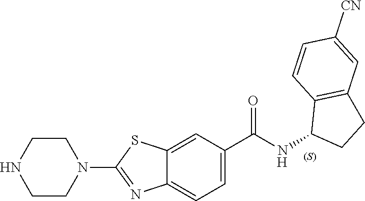 |
| Example 114 | (R)-N-(5-cyano-2,3- dihydro-1H-inden-1- yl)-2-(piperazin-1- yl)benzo[d]thiazole- 6-carboxamide | 403.5 | 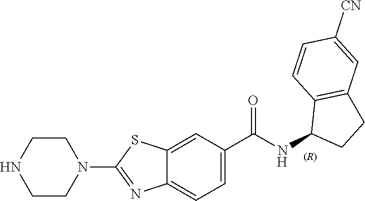 |
| Example 115 | (S)-N-(4-cyano-2,3- dihydro-1H-inden- 1-yl)-2-(piperazin-1- yl)benzo[d]thiazole- 6-carboxamide | 403.5 | 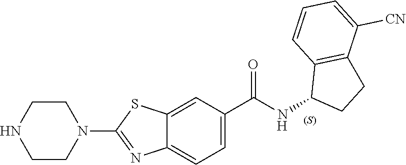 |
| Example 116 | (R)-N-(4-cyano-2,3- dihydro-1H-inden-1- yl)-2-(piperazin-1- yl)benzo[d]thiazole- 6-carboxamide | 403.5 | 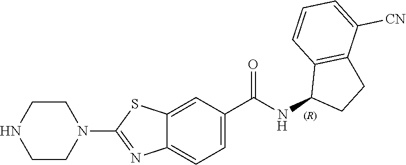 |
| Example 117 | N-cyclopentyl-2- (piperazin-1-yl)benzo- [d]thiazole-6-carbox- amide | 330.5 | 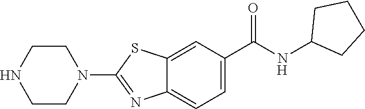 |
| Example 118 | N-cyclohexyl-2- (piperazin-1-yl)benzo- [d]thiazole-6-carbox- amide | 344.5 | 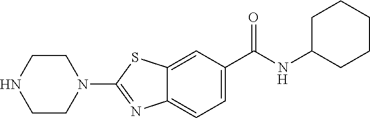 |
| Example 119 | N-(4,4-difluorocyclo- hexyl)-2-(piperazin-1- yl)benzo[d]thiazole- 6-carboxamide | 380.5 | 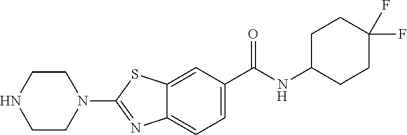 |
| Example 120 | (S)-N-(chroman-4-yl)- 2-(4-(2-hydroxyethyl)- piperazin-1-yl)benzo- [d]thiazole-6-carbox- amide | 438.6 | 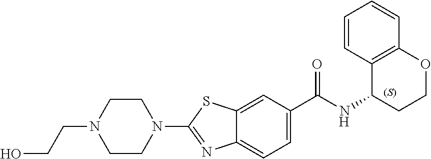 |
| Example 121 | 2-(3,8-diazabicyclo- [3.2.1]octan-3-yl)- N-((S)-chroman-4- yl)benzo[d]thiazole- 6-carboxamide | 420.5 | 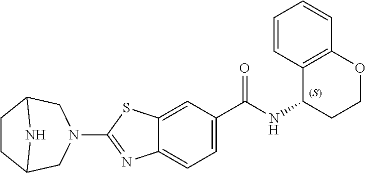 |
| Example 122 | 2-(3,8-diazabicyclo- [3.2.1]octan-8-yl)- N-((S)-chroman-4- yl)benzo[d]thiazole- 6-carboxamide | 420.5 | 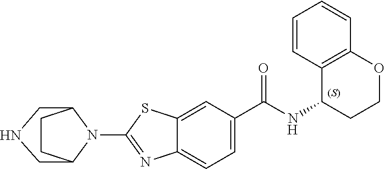 |
| Example 123 | N-(4-chlorobenzyl)- 2-(piperidin-4-yl)- benzo[d]thiazole-6- carboxamide | 385.9 | 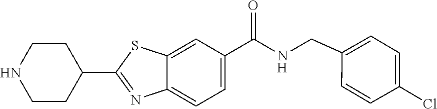 |
| Example 124 | N-(4-fluorobenzyl)- 2-(piperidin-4-yl)- benzo[d]thiazole-6- carboxamide | 369.5 | 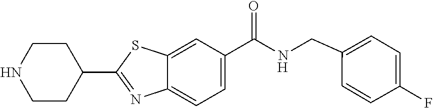 |
| Example 125 | N-(4-methoxybenzyl)- 2-(piperidin-4-yl)- benzo[d]thiazole-6- carboxamide | 381.5 | 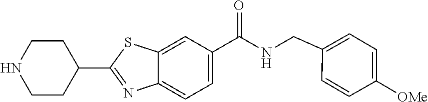 |
| Example 126 | N-(4-cyanobenzyl)- 2-(piperidin-4-yl)- benzo[d]thiazole-6- carboxamide | 376.5 | 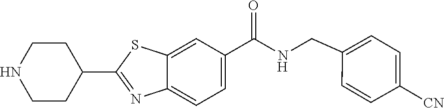 |
| Example 127 | (S)-N-(2,3-dihydro- 1H-inden-1-yl)-2- (piperidin-4-yl)- benzo[d]thiazole-6- carboxamide | 377.5 | 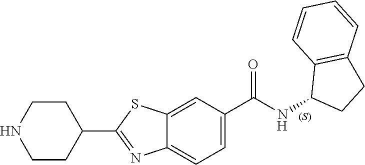 |
| Example 128 | N-((1R,2R)-2-hydroxy- 2,3-dihydro-1H-inden- 1-yl)-2-(piperidin-4- yl)benzo[d]thiazole-6- carboxamide | 393.5 | 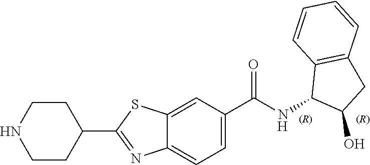 |
| Example 129 | N-((1S,2S)-2-hydroxy- 2,3-dihydro-1H-inden- 1-yl)-2-(piperidin-4- yl)benzo[d]thiazole- 6-carboxamide | 393.5 | 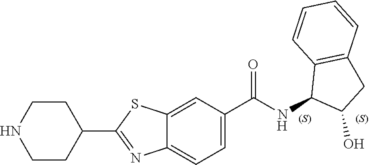 |
| Example 130 | N-((1R,2S)-2-hydroxy- 2,3-dihydro-1H-inden- 1-yl)-2-(piperidin-4- yl)benzo[d]thiazole- 6-carboxamide | 393.5 | 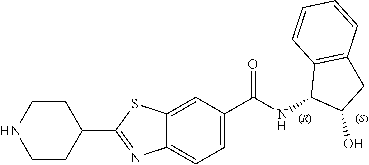 |
| Example 131 | N-((1S,2R)-2-hydroxy- 2,3-dihydro-1H-inden- 1-yl)-2-(piperidin-4- yl)benzo[d]thiazole- 6-carboxamide | 393.5 | 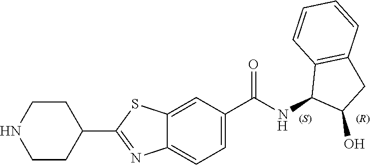 |
| Example 132 | (S)-N-(chroman-4-yl)- 2-(piperidin-4-yl)- benzo[d]thiazole-6- carboxamide | 393.5 | 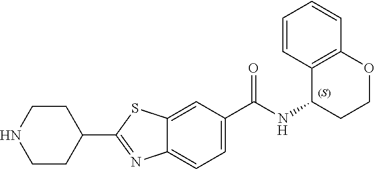 |
| Example 133 | N-isopropyl-2- (piperidin-4-yl)benzo- [d]thiazole-6-carbox- amide | 303.4 | 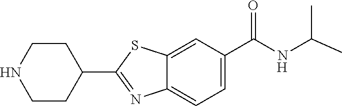 |
| Example 134 | N-cyclopentyl-2- (piperidin-4-yl)benzo- [d]thiazole-6-carbox- amide | 329.5 | 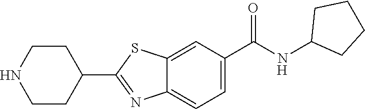 |
| Example 135 | N-cyclohexyl-2- (piperidin-4-yl)benzo- [d]thiazole-6-carbox- amide | 343.5 | 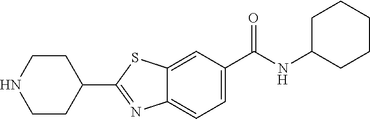 |
| Example 136 | N-(4,4-difluorocyclo- hexyl)-2-(piperidin-4- yl)benzo[d]thiazole-6- carboxamide | 379.5 | 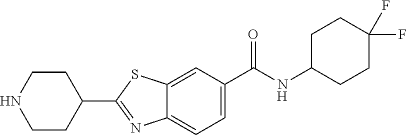 |
| Example 137 | N-(3,3-difluorocyclo- butyl)-2-(piperidin-4- yl)benzo[d]thiazole-6- carboxamide | 351.4 | 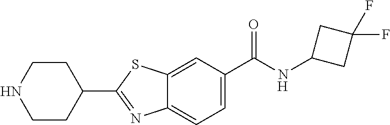 |
| Example 138 | 2-(piperidin-4-yl)-N- (tetrahydro-2H-pyran- 4-yl)benzo[d]thiazole- 6-carboxamide | 345.5 | 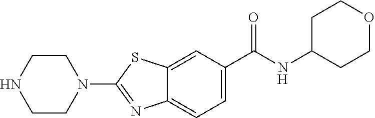 |
| Example 139 | (S)-N-(2-methoxy-1- phenylethyl)-2- (piperidin-4-yl)benzo- [d]thiazole-6-carbox- amide | 395.5 | 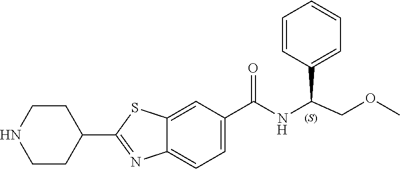 |
| Example 140 | (R)-N-(2-methoxy- 1-phenylethyl)-2- (piperidin-4-yl)- benzo[d]thiazole-6- carboxamide | 395.5 | 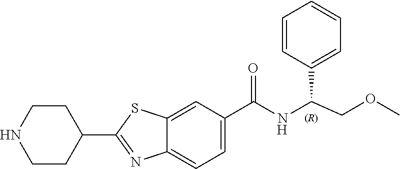 |
| Example 141 | (R)-N-(2-hydroxy- 1-phenylethyl)-2- (piperidin-4-yl)- benzo[d]thiazole-6- carboxamide | 381.5 | 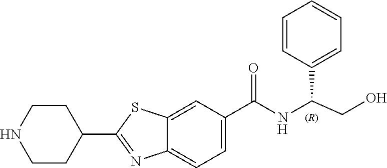 |
| Example 142 | N-benzyl-2-(1- methylpiperidin-4- yl)benzo[d]thiazole- 6-carboxamide | 365.5 | 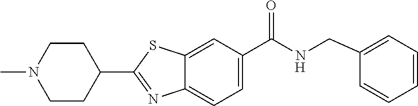 |
| Example 143 | N-(4-chlorobenzyl)- 2-(1-methylpiper- idin-4-yl)benzo[d]- thiazole-6-carbox- amide | 399.9 | 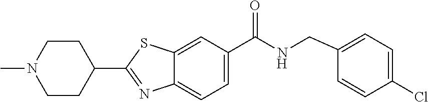 |
| Example 144 | N-(4-fluorobenzyl)- 2-(1-methylpiperidin- 4-yl)benzo[d]thiazole- 6-carboxamide | 383.5 | 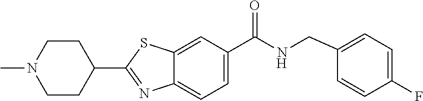 |
| Example 145 | N-(4-methoxybenzyl)- 2-(1-methylpiperidin- 4-yl)benzo[d]thiazole- 6-carboxamide | 395.5 | 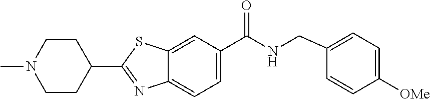 |
| Example 146 | N-(4-cyanobenzyl)- 2-(1-methylpiperidin- 4-yl)benzo[d]thiazole- 6-carboxamide | 390.5 | 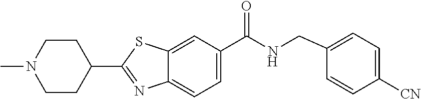 |
| Example 147 | N-(4-methylbenzyl)- 2-(1-methylpiperidin- 4-yl)benzo[d]thiazole- 6-carboxamide | 379.5 | 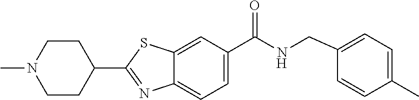 |
| Example 148 | N-((1R,2S)-2-hydroxy- 2,3-dihydro-1H-inden- 1-yl)-2-(1-methylpiper- idin-4-yl)benzo[d]- thiazole-6-carboxamide | 407.5 | 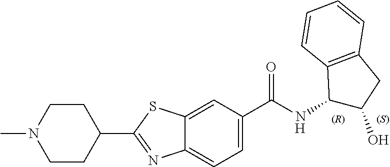 |
| Example 149 | N-((1R,2R)-2-hydroxy- 2,3-dihydro-1H-inden- 1-yl)-2-(1-methylpiper- idin-4-yl)benzo[d]- thiazole-6-carboxamide | 407.5 | 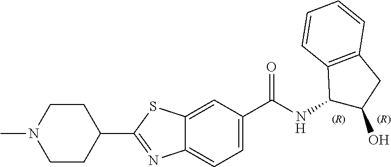 |
| Example 150 | (S)-N-(chroman-4-yl)- 2-(1-methylpiperidin- 4-yl)benzo[d]thiazole- 6-carboxamide | 407.5 | 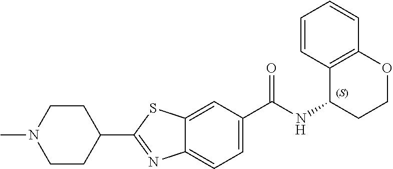 |
| Example 151 | N-cyclopentyl-2-(1- methylpiperidin-4- yl)benzo[d]thiazole- 6-carboxamide | 343.5 | 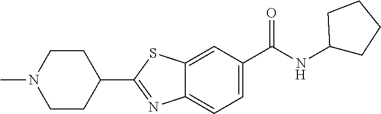 |
| Example 152 | 2-(1-methylpiperidin- 4-yl)-N-(tetrahydro- 2H-pyran-4-yl)benzo- [d]thiazole-6-carbox- amide | 359.5 | 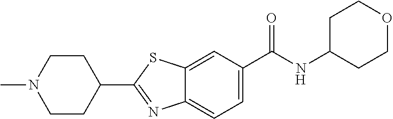 |
| Example 153 | N-cyclohexyl-2-(1- methylpiperidin-4- yl)benzo[d]thiazole- 6-carboxamide | 357.5 | 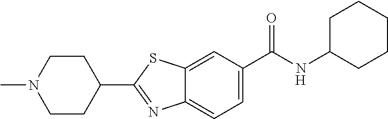 |
| Example 154 | N-(4,4-difluorocyclo- hexyl)-2-(1-methyl- piperidin-4-yl)benzo- [d]thiazole-6-carbox- amide | 393.5 | 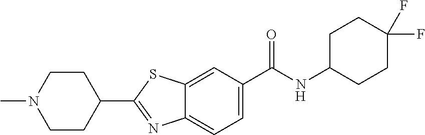 |
| Example 155 | N-isopropyl-2-(1- methylpiperidin-4- yl)benzo[d]thiazole- 6-carboxamide | 317.5 | 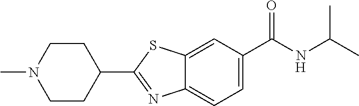 |
| Example 156 | N-cyclobutyl-2-(1- methylpiperidin-4- yl)benzo[d]thiazole- 6-carboxamide | 329.5 | 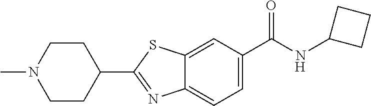 |
| Example 157 | N-(3,3-difluorocyclo- butyl)-2-(1-methyl- piperidin-4-yl)benzo- [d]thiazole-6-carbox- amide | 365.4 | 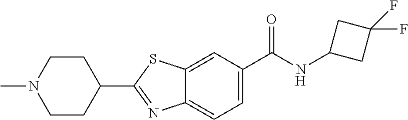 |
| Example 158 | (S)-N-(2-methoxy- 1-phenylethyl)-2- (1-methylpiperidin- 4-yl)benzo[d]thiazole- 6-carboxamide | 409.6 | 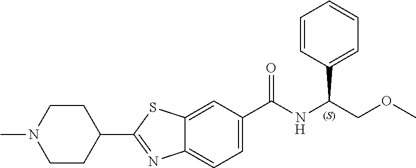 |
| Example 159 | (R)-N-(2-methoxy- 1-phenylethyl)-2- (1-methylpiperidin- 4-yl)benzo[d]thiazole- 6-carboxamide | 409.6 | 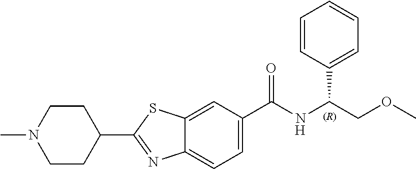 |
| Example 160 | (S)-N-(2-hydroxy- 1-phenylethyl)-2- (1-methylpiperidin- 4-yl)benzo[d]thiazole- 6-carboxamide | 395.5 | 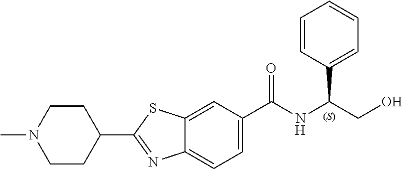 |
| Example 161 | (R)-N-(2-hydroxy- 1-phenylethyl)-2-(1- methylpiperidin-4- yl)benzo[d]thiazole- 6-carboxamide | 395.5 | 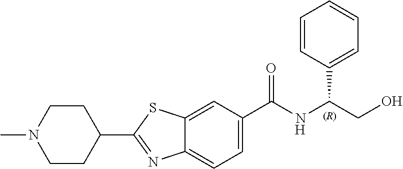 |
| Example 162 | N-cyclopentyl-2-(1- ethylpiperidin-4-yl)- benzo[d]thiazole-6- carboxamide | 357.5 | 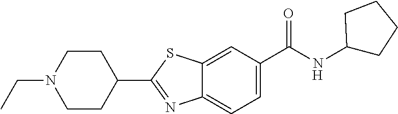 |
| Example 163 | 2-(1-ethylpiperidin- 4-yl)-N-(tetrahydro- 2H-pyran-4-yl)benzo- [d]thiazole-6-carbox- amide | 373.5 | 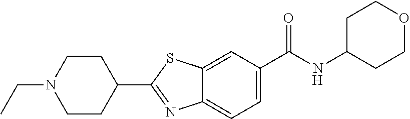 |
| Example 164 | 2-(1-ethylpiperidin- 4-yl)-N-((1R,2R)- 2-hydroxy-2,3- dihydro-1H-inden- 1-yl)benzo[d]thiazole- 6-carboxamide | 421.6 | 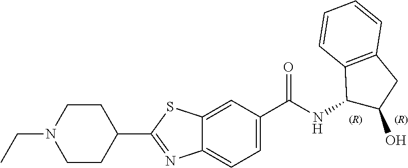 |
| Example 165 | 2-(1-ethylpiperidin- 4-yl)-N-((1S,2S)-2- hydroxy-2,3-dihydro- 1H-inden-1-yl)benzo- [d]thiazole-6-carbox- amide | 421.6 | 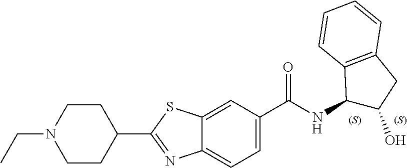 |
| Example 166 | 2-(1-ethylpiperidin- 4-yl)-N-((1R,2S)-2- hydroxy-2,3-dihydro- 1H-inden-1-yl)benzo- [d]thiazole-6-carbox- amide | 421.6 | 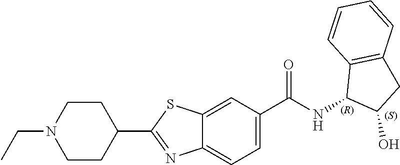 |
| Example 167 | 2-(1-ethylpiperidin- 4-yl)-N-((1S,2R)-2- hydroxy-2,3-dihydro- 1H-inden-1-yl)benzo- [d]thiazole-6-carbox- amide | 421.6 | 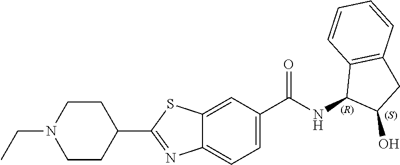 |
| Example 168 | (S)-N-(chroman-4-yl)- 2-(1-ethylpiperidin- 4-yl)benzo[d]thiazole- 6-carboxamide | 421.6 | 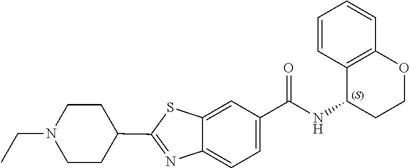 |
| Example 169 | (S)-N-(2,3-dihydro- 1H-inden-1-yl)-2- (1-(2-hydroxyethyl)- piperidin-4-yl)benzo- [d]thiazole-6-carbox- amide | 421.6 | 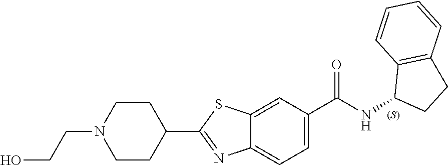 |
| Example 170 | (S)-N-(chroman-4- yl)-2-(1-(2-hydroxy- ethyl)piperidin-4- yl)benzo[d]thiazole- 6-carboxamide | 437.6 | 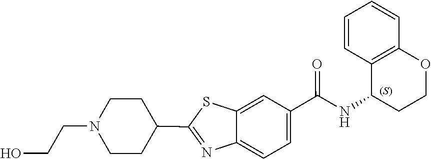 |
| Example 171 | N-cyclopentyl-2-(1-(2- hydroxyethyl)piperidin- 4-yl)benzo[d]thiazole- 6-carboxamide | 373.5 | 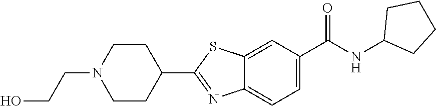 [SMILES: O=C(NC1CCCC1)c1ccc2nc(C3CCN(CCO)CC3)sc2c1] |
| Example 172 | 2-(1-(2-hydroxy- ethyl)piperidin-4-yl)- N-(tetrahydro-2H- pyran-4-yl)benzo[d]- thiazole-6-carbox- amide | 389.5 | 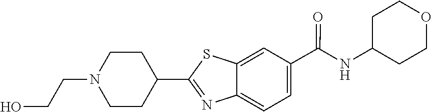 [SMILES: O=C(NC1CCOCC1)c1ccc2nc(C3CCN(CCO)CC3)sc2c1] |
| Example 173 | N-cyclohexyl-2-(1- (2-hydroxyethyl)- piperidin-4-yl)benzo- [d]thiazole-6-carbox- amide | 387.5 | 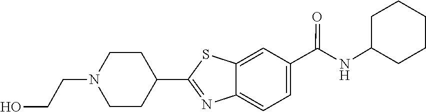 [SMILES: O=C(NC1CCCCC1)c1ccc2nc(C3CCN(CCO)CC3)sc2c1] |
| Example 174 | N-(4,4-difluorocyclo- hexyl)-2-(1-(2-hydroxy- ethyl)piperidin-4-yl)- benzo[d]thiazole-6- carboxamide | 423.5 | 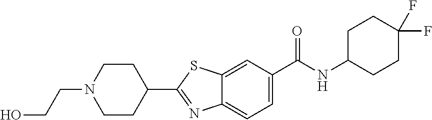 [SMILES: O=C(NC1CCC(F)(F)CC1)c1ccc2nc(C3CCN(CCO)CC3)sc2c1] |
| Example 175 | (S)-N-(chroman-4-yl)- 2-(1-(2-(2-methoxy- ethoxy)ethyl)piperidin- 4-yl)benzo[d]thiazole- 6-carboxamide | 495.6 | 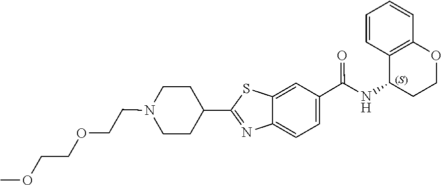 |
| Example 176 | N-((S)-chroman-4- yl)-2-((S)-piperidin- 3-yl)benzo[d]thiazole- 6-carboxamide | 393.5 | 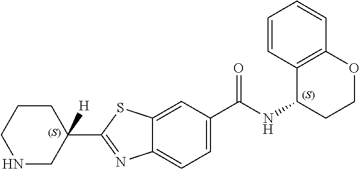 |
| Example 177 | N-((S)-chroman-4- yl)-2-((R)-piperidin- 3-yl)benzo[d]thiazole- 6-carboxamide | 393.5 | 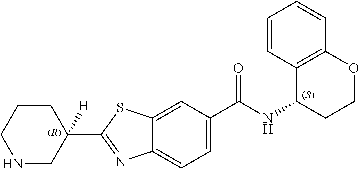 |
| Example 178 | N-((S)-chroman-4-yl)- 2-(pyrrolidin-3-yl)- benzo[d]thiazole-6- carboxamide | 379.5 | 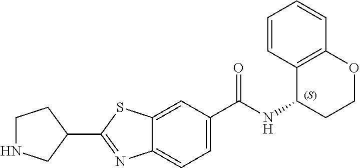 |
| Example 179 | (S)-N-(2,3-dihydro- 1H-inden-1-yl)-4- methyl-2-(piperidin- 4-yl)benzo[d]thiazole- 6-carboxamide | 391.5 | 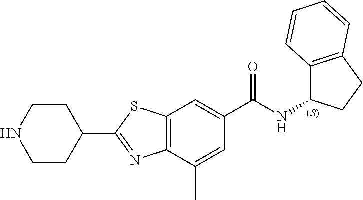 |
| Example 180 | (S)-N-(2,3-dihydro- 1H-inden-1-yl)-2- (1-ethylpiperidin-4- yl)-4-methylbenzo- [d]thiazole-6-carbox- amide | 419.6 | 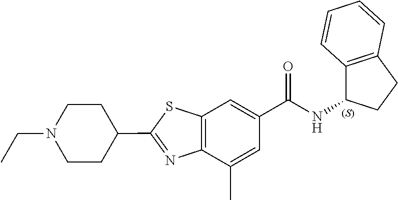 |
| Example 181 | N-cyclopentyl-4- methyl-2-(piperidin- 4-yl)benzo[d]thiazole- 6-carboxamide | 343.5 | 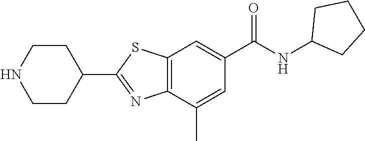 [SMILES: Cc1cc(C(=O)NC2CCCC2)cc2sc(C3CCNCC3)nc12] |
| Example 182 | N-cyclopentyl-2-(1- ethylpiperidin-4-yl)- 4-methylbenzo[d]- thiazole-6-carboxamide | 371.5 | 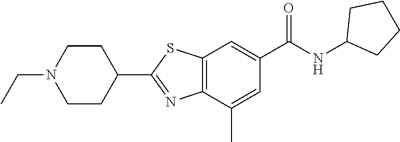 [SMILES: CCN1CCC(c2nc3c(C)cc(C(=O)NC4CCCC4)cc3s2)CC1] |
| Example 183 | N-cyclopentyl-4- methyl-2-(1-methyl- piperidin-4-yl)benzo- [d]thiazole-6-carbox- amide | 357.5 | 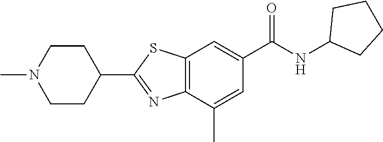 [SMILES: Cc1cc(C(=O)NC2CCCC2)cc2sc(C3CCN(C)CC3)nc12] |
| Example 184 | 4-methyl-2-(piperidin- 4-yl)-N-(tetrahydro- 2H-pyran-4-yl)benzo- [d]thiazole-6-carbox- amide | 359.5 | 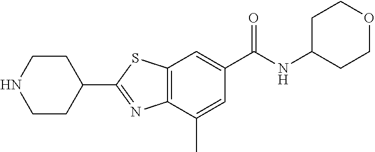 [SMILES: Cc1cc(C(=O)NC2CCOCC2)cc2sc(C3CCNCC3)nc12] |
| Example 185 | 2-(1-ethylpiperidin-4- yl)-4-methyl-N-(tetra- hydro-2H-pyran-4-yl)- benzo[d]thiazole-6- carboxamide | 387.5 | 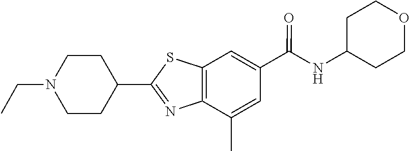 [SMILES: CCN1CCC(c2nc3c(C)cc(C(=O)NC4CCOCC4)cc3s2)CC1] |
| Example 186 | (S)-N-(chroman-4-yl)- 4-methyl-2-(piperidin- 4-yl)benzo[d]thiazole- 6-carboxamide | 407.5 | 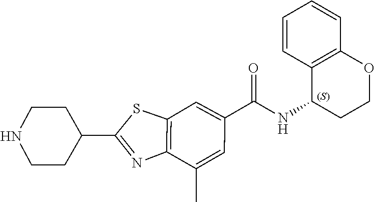 |
| Example 187 | (S)-N-(chroman-4-yl)- 2-(1-ethyl-piperidin-4-yl)-4-methylbenzo[d]- thiazole-6-carboxamide | 435.6 | 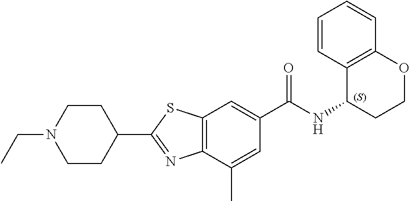 |
| Example 188 | N-cyclopentyl-5-meth- yl-2-(piperidin-4-yl)- benzo[d]thiazole-6- carboxamide | 343.5 | 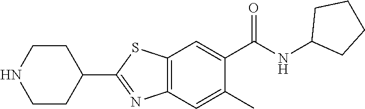 [SMILES: Cc1cc2nc(C3CCNCC3)sc2cc1C(=O)NC1CCCC1] |
| Example 189 | N-cyclopentyl-2-(1- ethylpiperidin-4-yl)- 5-methylbenzo[d]- thiazole-6-carboxamide | 371.5 | 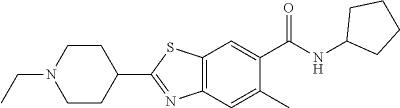 [SMILES: CCN1CCC(c2nc3cc(C)c(C(=O)NC4CCCC4)cc3s2)CC1] |
| Example 190 | (S)-N-(chroman-4-yl)- 5-methyl-2-(piperidin- 4-yl)benzo[d]thiazole- 6-carboxamide | 407.5 | 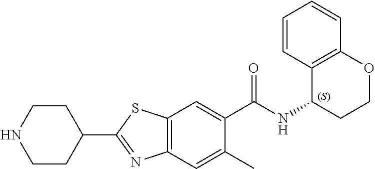 |
| Example 191 | (S)-N-(chroman-4-yl)- 2-(1-ethylpiperidin-4- yl)-5-methylbenzo[d]- thiazole-6-carboxamide | 435.6 | 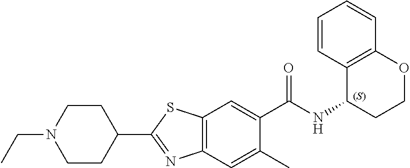 |
| Example 192 | N-cyclopentyl-7- methyl-2-(piperidin-4- yl)benzo[d]thiazole-6- carboxamide | 343.5 | 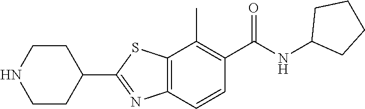 [SMILES: Cc1c(C(=O)NC2CCCC2)ccc2nc(C3CCNCC3)sc12] |
| Example 193 | N-cyclopentyl-2-(1- ethylpiperidin-4-yl)- 7-methylbenzo[d]- thiazole-6-carboxamide | 371.5 | 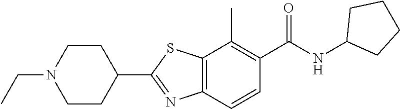 [SMILES: CCN1CCC(c2nc3ccc(C(=O)NC4CCCC4)c(C)c3s2)CC1] |
| Example 194 | (S)-N-(chroman-4-yl)- 7-methyl-2-(piperidin- 4-yl)benzo[d]thiazole- 6-carboxamide | 407.5 | 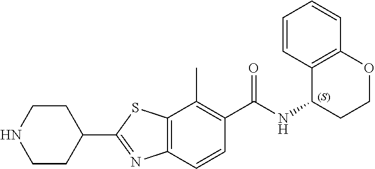 |
| Example 195 | (S)-N-(chroman-4-yl)- 2-(1-ethylpiperidin-4- yl)-7-methylbenzo[d]- thiazole-6-carboxamide | 435.6 | 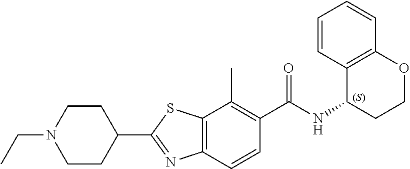 |
| Example 196 | (S)-N-(2,3-dihydro- 1H-inden-1-yl)-2-(1- ethylpiperidin-4-yl)- 7-methylbenzo[d]- thiazole-6-carboxamide | 419.6 | 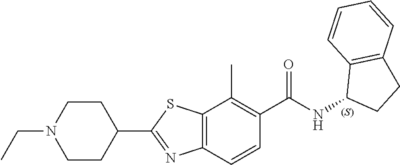 |
| Example 197 | 2-(1-ethylpiperidin-4- yl)-7-methyl-N-(tetra- hydro-2H-pyran-4-yl)- benzo[d]thiazole-6- carboxamide | 387.5 | 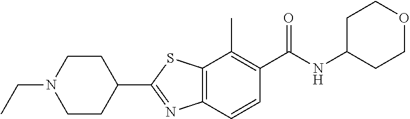 [SMILES: CCN1CCC(c2nc3ccc(C(=O)NC4CCOCC4)c(C)c3s2)CC1] |
| Example 198 | (S)-N-(2,3-dihydro- 1H-inden-1-yl)-5- methoxy-2-(piperidin- 4-yl)benzo[d]thiazole- 6-carboxamide | 407.5 | 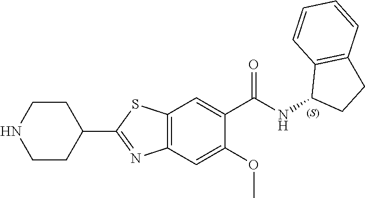 |
| Example 199 | N-cyclopentyl-5- methoxy-2-(piperidin- 4-yl)benzo[d]thiazole- 6-carboxamide | 359.5 | 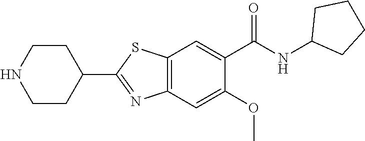 [SMILES: COc1cc2nc(C3CCNCC3)sc2cc1C(=O)NC1CCCC1] |
| Example 200 | 5-methoxy-2- (piperidin-4-yl)-N- (tetrahydro-2H-pyran- 4-yl)benzo[d]thiazole- 6-carboxamide | 375.5 | 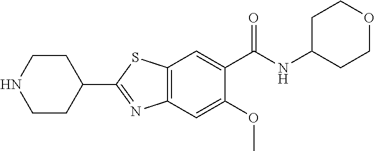 [SMILES: COc1cc2nc(C3CCNCC3)sc2cc1C(=O)NC1CCOCC1] |
| Example 201 | (S)-N-(chroman-4- yl)-5-methoxy-2- (piperidin-4-yl)benzo- [d]thiazole-6-carbox- amide | 423.5 | 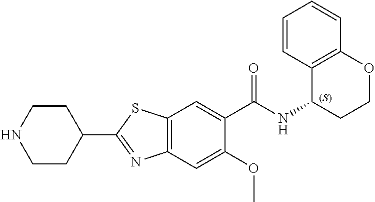 |
| Example 202 | (S)-N-(2,3-dihydro- 1H-inden-1-yl)-4- methyl-2-(piperazin- 1-yl)benzo[d]thiazole- 6-carboxamide | 392.5 | 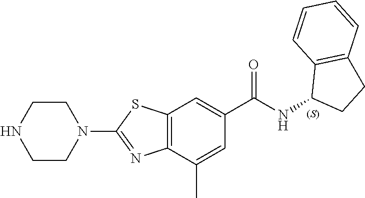 |
| Example 203 | (S)-N-(2,3-dihydro- 1H-inden-1-yl)-2- (4-ethylpiperazin- 1-yl)-4-methylbenzo- [d]thiazole-6-carbox- amide | 420.6 | 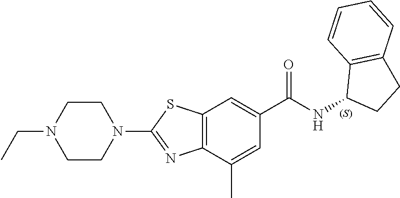 |
| Example 204 | N-cyclobutyl-4- methyl-2-(piperazin- 1-yl)benzo[d]thiazole- 6-carboxamide | 330.4 | 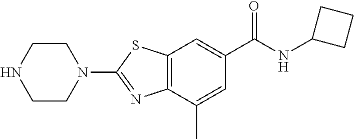 [SMILES: Cc1cc(C(=O)NC2CCC2)cc2sc(N3CCNCC3)nc12] |
| Example 205 | N-cyclobutyl-4- methyl-2-(4-methyl- piperazin-1-yl)benzo- [d]thiazole-6-carbox- amide | 344.5 | 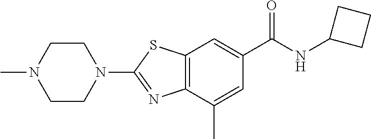 [SMILES: Cc1cc(C(=O)NC2CCC2)cc2sc(N3CCN(C)CC3)nc12] |
| Example 206 | N-cyclopentyl-4- methyl-2-(piperazin-1- yl)benzo[d]thiazole-6- carboxamide | 344.5 | 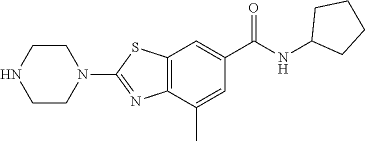 [SMILES: Cc1cc(C(=O)NC2CCCC2)cc2sc(N3CCNCC3)nc12] |
| Example 207 | N-cyclopentyl-2-(4- ethylpiperazin-1-yl)- 4-methylbenzo[d]- thiazole-6-carboxamide | 372.5 | 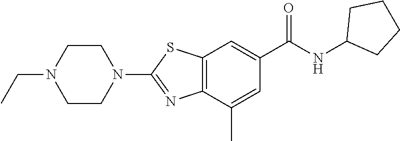 [SMILES: CCN1CCN(c2nc3c(C)cc(C(=O)NC4CCCC4)cc3s2)CC1] |
| Example 208 | N-cyclopentyl-4- methyl-2-(4-methyl- piperazin-1-yl)benzo- [d]thiazole-6-carbox- amide | 358.5 | 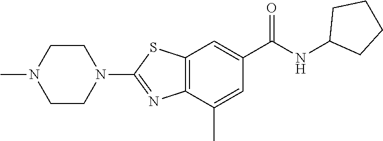 [SMILES: Cc1cc(C(=O)NC2CCCC2)cc2sc(N3CCN(C)CC3)nc12] |
| Example 209 | 4-methyl-2-(piperazin- 1-yl)-N-(tetrahydro- 2H-pyran-4-yl)benzo- [d]thiazole-6-carbox- amide | 360.5 | 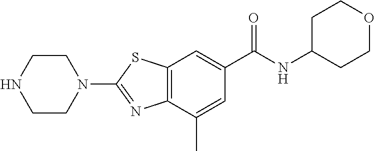 [SMILES: Cc1cc(C(=O)NC2CCOCC2)cc2sc(N3CCNCC3)nc12] |
| Example 210 | 2-(4-ethylpiperazin-1- yl)-4-methyl-N-(tetra- hydro-2H-pyran-4- yl)benzo[d]thiazole- 6-carboxamide | 388.5 | 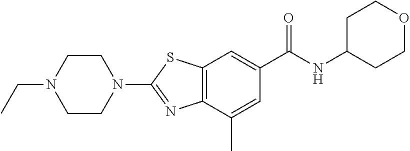 [SMILES: CCN1CCN(c2nc3c(C)cc(C(=O)NC4CCOCC4)cc3s2)CC1] |
| Example 211 | (S)-N-(chroman-4-yl)- 4-methyl-2-(piperazin- 1-yl)benzo[d]thiazole- 6-carboxamide | 408.5 | 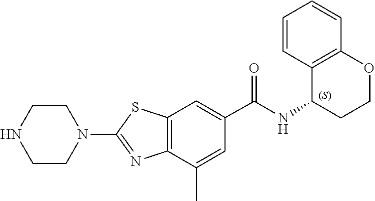 |
| Example 212 | (S)-N-(chroman-4-yl)- 2-(4-ethylpiperazin-1- yl)-4-methylbenzo[d]- thiazole-6-carboxamide | 436.6 | 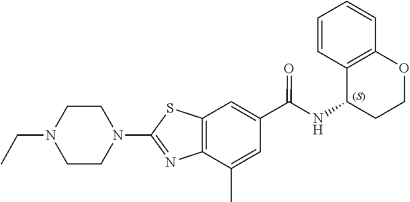 |
| Example 213 | N-cyclopentyl-5-meth- yl-2-(piperazin-1-yl)- benzo[d]thiazole-6- carboxamide | 344.5 | 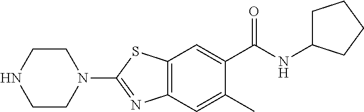 [SMILES: Cc1cc2nc(N3CCNCC3)sc2cc1C(=O)NC1CCCC1] |
| Example 214 | N-cyclopentyl-2-(4- ethylpiperazin-1-yl)- 5-methylbenzo[d]- thiazole-6-carboxamide | 372.5 | 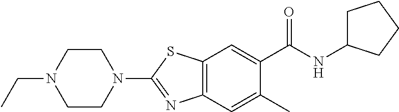 [SMILES: CCN1CCN(c2nc3cc(C)c(C(=O)NC4CCCC4)cc3s2)CC1] |
| Example 215 | (S)-N-(chroman-4-yl)- 5-methyl-2-(piperazin- 1-yl)benzo[d]thiazole- 6-carboxamide | 408.5 | 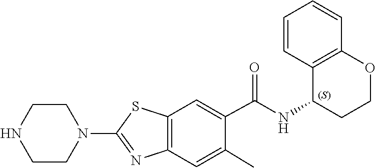 |
| Example 216 | (S)-N-(chroman-4-yl)- 2-(4-ethylpiperazin-1- yl)-5-methylbenzo[d]- thiazole-6-carboxamide | 436.6 | 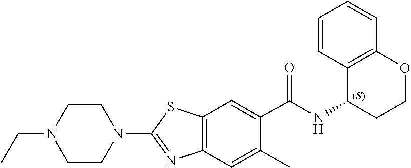 |
| Example 217 | N-cyclopentyl-7- methyl-2-(piperazin-1- yl)benzo[d]thiazole-6- carboxamide | 344.5 | 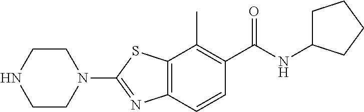 [SMILES: Cc1c(C(=O)NC2CCCC2)ccc2nc(N3CCNCC3)sc12] |
| Example 218 | N-cyclopentyl-2-(4- ethylpiperazin-1-yl)- 7-methylbenzo[d]- thiazole-6-carboxamide | 372.5 | 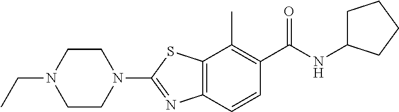 [SMILES: CCN1CCN(c2nc3ccc(C(=O)NC4CCCC4)c(C)c3s2)CC1] |
| Example 219 | (S)-N-(chroman-4-yl)- 7-methyl-2-(piperazin- 1-yl)benzo[d]thiazole- 6-carboxamide | 408.5 | 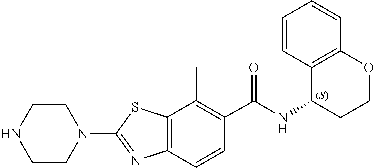 |
| Example 220 | (S)-N-(chroman-4-yl)- 2-(4-ethylpiperazin-1- yl)-7-methylbenzo[d]- thiazole-6-carboxamide | 436.6 | 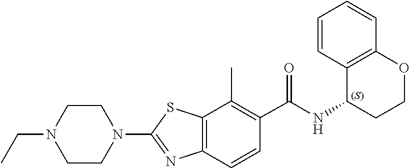 |
| Example 221 | N-cyclopentyl-4-meth- oxy-2-(piperazin-1- yl)benzo[d]thiazole-6- carboxamide | 360.5 | 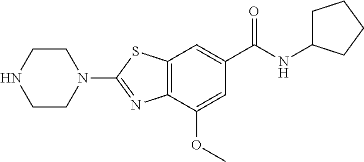 [SMILES: COc1cc(C(=O)NC2CCCC2)cc2sc(N3CCNCC3)nc12] |
| Example 222 | N-cyclopentyl-2-(4- ethylpiperazin-1-yl)- 4-methoxybenzo[d]- thiazole-6-carboxamide | 388.5 | 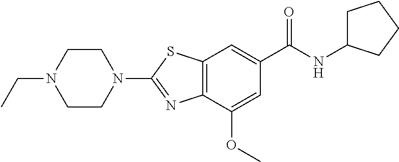 [SMILES: CCN1CCN(c2nc3c(OC)cc(C(=O)NC4CCCC4)cc3s2)CC1] |
| Example 223 | (S)-N-(chroman-4- yl)-4-methoxy-2- (piperazin-1-yl)benzo- [d]thiazole-6-carbox- amide | 424.5 | 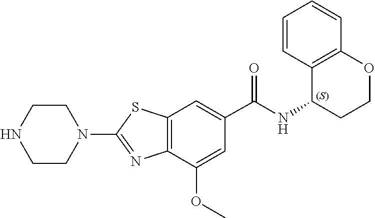 |
| Example 224 | (S)-N-(2,3-dihydro- 1H-inden-1-yl)-5- methoxy-2-(piperazin- 1-yl)benzo[d]thiazole- 6-carboxamide | 408.5 | 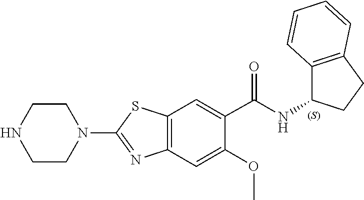 |
| Example 225 | N-cyclopentyl-5- methoxy-2-(piperazin- 1-yl)benzo[d]thiazole- 6-carboxamide | 360.5 | 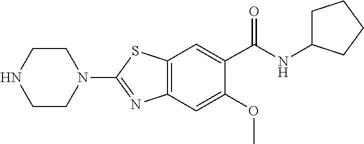 [SMILES: COc1cc2nc(N3CCNCC3)sc2cc1C(=O)NC1CCCC1] |
| Example 226 | N-cyclopentyl-2-(4- ethylpiperazin-1-yl)- 5-methoxybenzo[d]- thiazole-6-carboxamide | 388.5 | 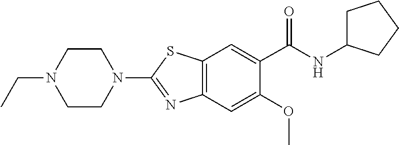 [SMILES: CCN1CCN(c2nc3cc(OC)c(C(=O)NC4CCCC4)cc3s2)CC1] |
| Example 227 | 5-methoxy-2- (piperazin-1-yl)-N- (tetrahydro-2H-pyran- 4-yl)benzo[d]thiazole- 6-carboxamide | 376.5 | 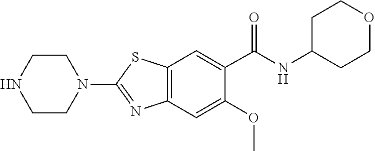 [SMILES: COc1cc2nc(N3CCNCC3)sc2cc1C(=O)NC1CCOCC1] |
| Example 228 | 2-(4-ethylpiperazin-1- yl)-5-methoxy-N- (tetrahydro-2H-pyran- 4-yl)benzo[d]thiazole- 6-carboxamide | 404.5 | 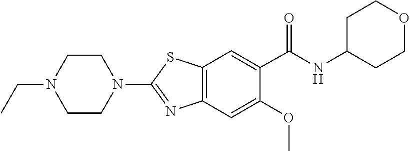 [SMILES: CCN1CCN(c2nc3cc(OC)c(C(=O)NC4CCOCC4)cc3s2)CC1] |
| Example 229 | (S)-N-(chroman-4-yl)- 5-methoxy-2-(piper- azin-1-yl)benzo[d]- thiazole-6-carboxamide | 424.5 | 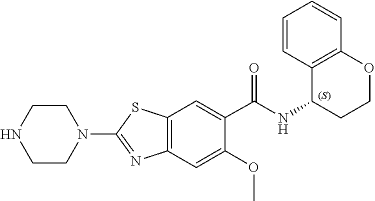 |
| Example 230 | (S)-N-(chroman-4-yl)- 4,7-dimethyl-2-(piper- idin-4-yl)benzo[d]- thiazole-6-carboxamide | 421.6 | 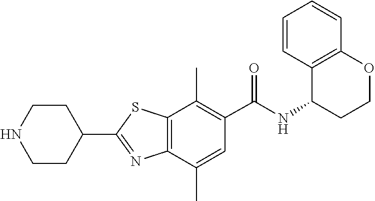 [SMILES: Cc1cc(C(=O)N[C@H]2CCOc3ccccc32)c(C)c2sc(C3CCNCC3)nc12] |
| Example 231 | 4-chloro-N-cyclo- pentyl-2-(1-methyl- piperidin-4-yl)benzo- [d]thiazole-6-carbox- amide | 377.9 | 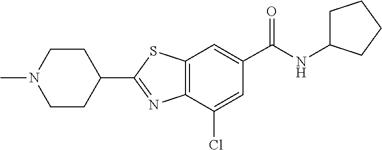 [SMILES: CN1CCC(c2nc3c(Cl)cc(C(=O)NC4CCCC4)cc3s2)CC1] |
| Example 232 | 4-cyclopropyl-N- isopropyl-2-(piperidin- 4-yl)benzo[d]thiazole- 6-carboxamide | 343.5 | 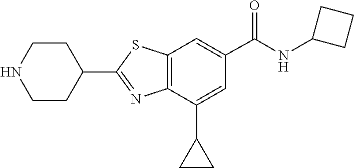 [SMILES: O=C(NC1CCC1)c1cc(C2CC2)c2nc(C3CCNCC3)sc2c1] |
| Example 233 | N-cyclobutyl-4-cyclo- opropyl-2-(1-meth- ylpiperidin-4-yl)- benzo[d]thiazole-6- carboxamide | 369.5 | 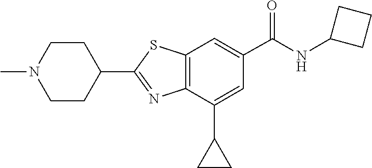 [SMILES: CN1CCC(c2nc3c(C4CC4)cc(C(=O)NC4CCC4)cc3s2)CC1] |
| Example 234 | 4-chloro-N-cyclo- butyl-2-(piperazin-1- yl)benzo-[d]thiazole- 6-carboxamide | 350.9 | 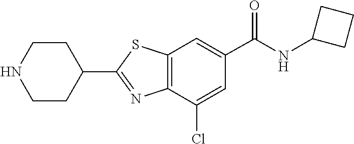 [SMILES: O=C(NC1CCC1)c1cc(Cl)c2nc(C3CCNCC3)sc2c1] |
| Example 235 | 4-chloro-N-cyclopent- yl-2-(4-(3-hydroxy- propyl)piperazin-1-yl)- benzo[d]thiazole-6- carboxamide | 423.0 | 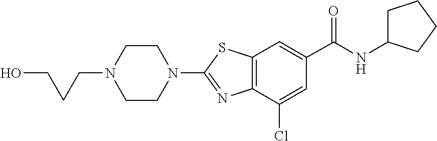 [SMILES: O=C(NC1CCCC1)c1cc(Cl)c2nc(N3CCN(CCCO)CC3)sc2c1] |
| Example 236 | 2-(3,8-diazabicyclo- [3.2.1]octan-8-yl)-N- cyclopentyl-7-meth- oxybenzo[d]thiazole- 6-carboxamide | 386.5 | 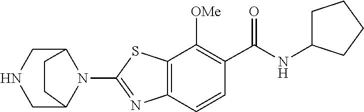 [SMILES: COc1c(C(=O)NC2CCCC2)ccc2nc(N3C4CCC3CNC4)sc12] |
| Example 237 | 2-(3,8-diazabicyclo- [3.2.1]octan-8-yl)-4- bromo-N-cyclopent- ylbenzo[d]thiazole-6- carboxamide | 435.4 | 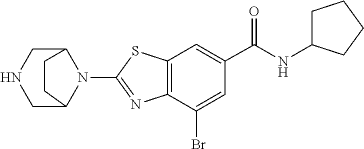 [SMILES: O=C(NC1CCCC1)c1cc(Br)c2nc(N3C4CCC3CNC4)sc2c1] |
| Example 238 | 2-(3,8-diazabicyclo- [3.2.1]octan-8-yl)-4- cyano-N-isopropyl- benzo[d]thiazole-6- carboxamide | 355.5 | 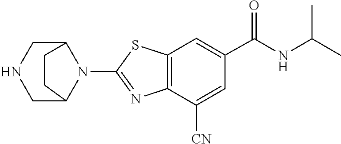 [SMILES: CC(C)NC(=O)c1cc(C#N)c2nc(N3C4CCC3CNC4)sc2c1] |
| Example 239 | 2-(3,8-diazabicyclo- [3.2.1]octan-8-yl)-N- cyclopentylbenzo[d]- thiazole-6-carboxamide | 356.5 | 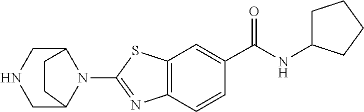 [SMILES: O=C(NC1CCCC1)c1ccc2nc(N3C4CCC3CNC4)sc2c1] |
| Example 240 | 2-(3,8-diazabicyclo- [3.2.1]octan-8-yl)-N- (4,4-difluorocyclohex- yl)benzo[d]thiazole-6- carboxamide | 406.5 | 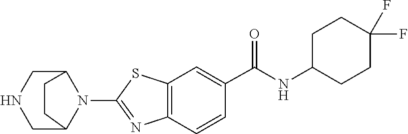 [SMILES: O=C(NC1CCC(F)(F)CC1)c1ccc2nc(N3C4CCC3CNC4)sc2c1] |
| Example 241 | 2-(3-cyclopropyl-3,8- diazabicyclo[3.2.1]- octan-8-yl)-N-(4,4- difluorocyclohexyl)- benzo[d]thiazole-6- carboxamide | 446.6 | 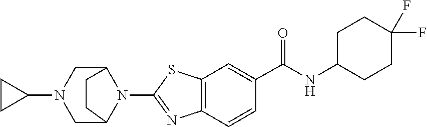 [SMILES: O=C(NC1CCC(F)(F)CC1)c1ccc2nc(N3C4CCC3CN(C3CC3)C4)sc2c1] |
| Example 242 | N-(4,4-difluorocyclo- hexyl)-2-(3-(2-hydroxy- ethyl)-3,8-diazabicyclo- [3.2.1]octan-8-yl)benzo- [d]thiazole-6-carbox- amide | 450.5 | 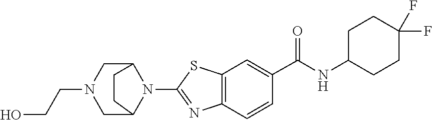 [SMILES: O=C(NC1CCC(F)(F)CC1)c1ccc2nc(N3C4CCC3CN(CCO)C4)sc2c1] |
| Example 243 | N-(4,4-difluoro- cyclohexyl)-2-(3-(3- hydroxypropyl)-3,8- diazabicyclo[3.2.1]- octan-8-yl)benzo[d]- thiazole-6-carbox- amide | 464.6 | 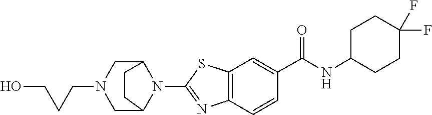 [SMILES: O=C(NC1CCC(F)(F)CC1)c1ccc2nc(N3C4CCC3CN(CCCO)C4)sc2c1] |
| Example 244 | 2-(3-oxa-7,9-diaza- bicyclo-[3.3.1]nonan- 9-yl)-N-cyclopentyl- benzo[d]thiazole-6- carboxamide | 372.5 | 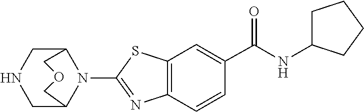 [SMILES: O=C(NC1CCCC1)c1ccc2nc(N3C4CNCC3COC4)sc2c1] |
| Example 245 | 2-(3,8-diazabicyclo- [3.2.1]octan-3-yl)-N- (4,4-difluorocyclo- hexyl)benzo[d]thiazole- 6-carboxamide | 406.5 | 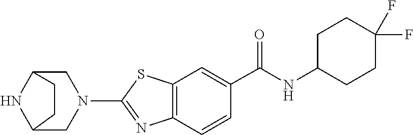 [SMILES: O=C(NC1CCC(F)(F)CC1)c1ccc2nc(N3CC4CCC(C3)N4)sc2c1] |
| Example 246 | N-(4,4-difluorocyclo- hexyl)-2-(8-methyl- 3,8-diazabicyclo[3.2.1]- octan-3-yl)benzo[d]- thiazole-6-carboxamide | 420.5 | 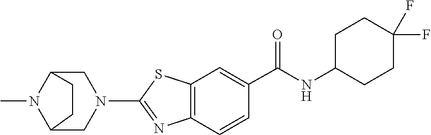 [SMILES: CN1C2CCC1CN(c1nc3ccc(C(=O)NC4CCC(F)(F)CC4)cc3s1)C2] |
| Example 247 | N-(4,4-difluorocyclo- hexyl)-2-(8-(2-hydroxyethyl)-3,8- diazabicyclo[3.2.1]- octan-3-yl)benzo[d]- thiazole-6-carboxamide | 450.5 | 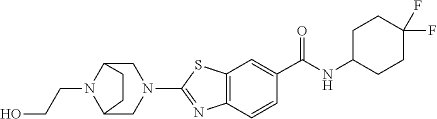 [SMILES: O=C(NC1CCC(F)(F)CC1)c1ccc2nc(N3CC4CCC(C3)N4CCO)sc2c1] |
| Example 248 | N-(4,4-difluorocyclo- hexyl)-2-(8-(3-fluoro- propyl)-3,8-diazabi- cyclo[3.2.1]octan-3-yl)- benzo[d]thiazole-6- carboxamide | 466.6 | 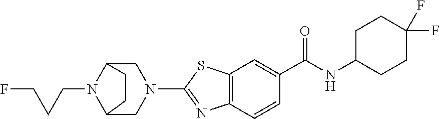 [SMILES: O=C(NC1CCC(F)(F)CC1)c1ccc2nc(N3CC4CCC(C3)N4CCCF)sc2c1] |
| Example 249 | 2-(3,8-diazabicyclo- [3.2.1]octan-8-yl)-N- cyclopentyl-4-(1- hydroxypropan-2-yl)- benzo[d]thiazole-6- carboxamide | 414.6 | 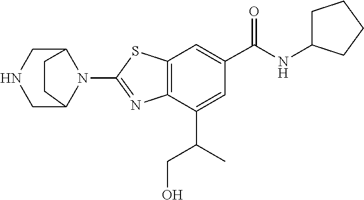 [SMILES: CC(CO)c1cc(C(=O)NC2CCCC2)cc2sc(N3C4CCC3CNC4)nc12] |
| Example 250 | 2-(3,8-diazabicyclo- [3.2.1]octan-8-yl)- N-cyclopentyl-4-(2- hydroxyethyl)benzo- [d]thiazole-6-carbox- amide | 400.5 | 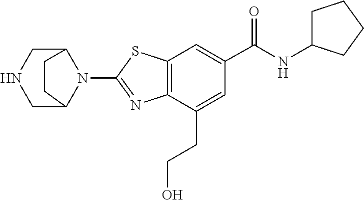 [SMILES: O=C(NC1CCCC1)c1cc(CCO)c2nc(N3C4CCC3CNC4)sc2c1] |
| Example 251 | (2-(piperazin-1-yl)- benzo[d]thiazol-6-yl)- (pyrrolidin-1-yl)meth- anone | 316.4 | 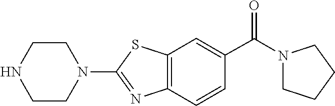 [SMILES: O=C(c1ccc2nc(N3CCNCC3)sc2c1)N1CCCC1] |
| Example 252 | 2-(3,6-diazabicyclo- [3.1.1]heptan-6-yl)- N-cyclopentylbenzo- [d]thiazole-6-carbox- amide | 342.5 | 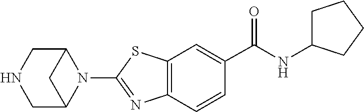 [SMILES: O=C(NC1CCCC1)c1ccc2nc(N3C4CNCC3C4)sc2c1] |
| Example 253 | N-cyclopentyl-2- (4,7-diazaspiro[2.5]- octan-7-yl)benzo- [d]thiazole-6-carbox- amide | 356.5 | 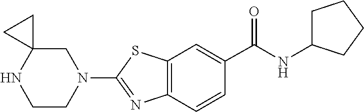 [SMILES: O=C(NC1CCCC1)c1ccc2nc(N3CCNC4(CC4)C3)sc2c1] |
| Example 254 | N-cyclohexyl-2- ((3aR,6aS)-hexa- hydropyrrolo[3,4-c]- pyrrol-2(1H)-yl)- benzo[d]thiazole-6- carboxamide | 370.5 | 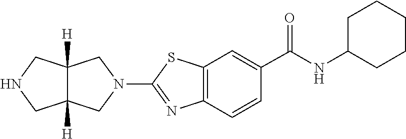 [SMILES: O=C(NC1CCCCC1)c1ccc2nc(N3C[C@H]4CNC[C@H]4C3)sc2c1] |
| Example 255 | N-cyclopentyl-2-(2,6- diazaspiro[3.3]heptan- 2-yl)benzo[d]thiazole- 6-carboxamide | 342.5 | 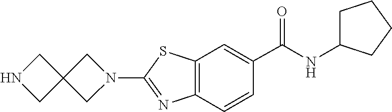 [SMILES: O=C(NC1CCCC1)c1ccc2nc(N3CC4(CNC4)C3)sc2c1] |
| Example 256 | N-cyclopentyl-2-(2,6- diazaspiro[3.4]octan-6- yl)benzo[d]thiazole- 6-carboxamide | 356.5 | 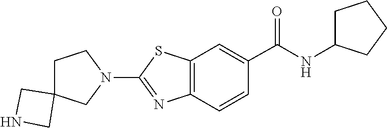 [SMILES: O=C(NC1CCCC1)c1ccc2nc(N3CCC4(CNC4)C3)sc2c1] |
| Example 257 | N-cyclopentyl-2-(2,7- diazaspiro[3.5]nonan- 7-yl)benzo[d]thiazole- 6-carboxamide | 370.5 | 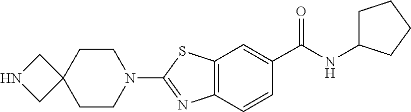 [SMILES: O=C(NC1CCCC1)c1ccc2nc(N3CCC4(CC3)CNC4)sc2c1] |
| Example 258 | N-cyclopentyl-2-(octa- hydro-4H-pyrrolo[3,2- b]pyridin-4-yl)-benzo- [d]thiazole-6-carbox- amide | 370.5 | 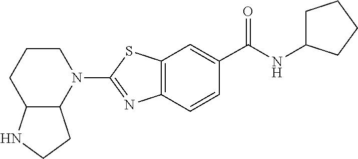 [SMILES: O=C(NC1CCCC1)c1ccc2nc(N3CCCC4NCCC43)sc2c1] |
| Example 259 | N-cyclopentyl-2-(octa- hydro-5H-pyrrolo[3,2- c]pyridin-5-yl)benzo- [d]thiazole-6-carbox- amide | 370.5 | 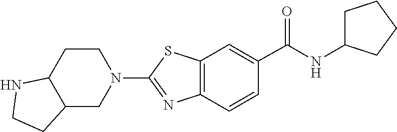 [SMILES: O=C(NC1CCCC1)c1ccc2nc(N3CCC4NCCC4C3)sc2c1] |
| Example 260 | N-cyclopentyl-2-(4- methyl-4,7-diazaspiro- [2.5]octan-7-yl)benzo- [d]thiazole-6-carbox- amide | 370.5 | 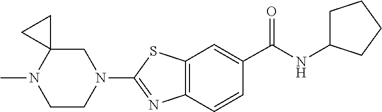 [SMILES: CN1CCN(c2nc3ccc(C(=O)NC4CCCC4)cc3s2)CC12CC2] |
| Example 261 | N-cyclohexyl-2- ((3aR,6aS)-5-methyl- hexahydropyrrolo- [3,4-c]pyrrol-2(1H)- yl)benzo[d]thiazole- 6-carboxamide | 384.5 | 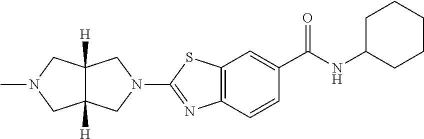 [SMILES: CN1C[C@@H]2CN(c3nc4ccc(C(=O)NC5CCCCC5)cc4s3)C[C@@H]2C1] |
| Example 262 | N-cyclopentyl-2-(6- methyl-2,6-diazaspiro- [3.3]heptan-2-yl)- benzo[d]thiazole-6- carboxamide | 356.5 | 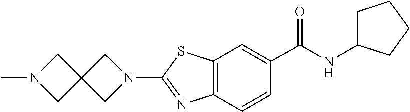 [SMILES: CN1CC2(C1)CN(c1nc3ccc(C(=O)NC4CCCC4)cc3s1)C2] |
| Example 263 | N-cyclopentyl-2-(2- methyl-2,6-diaza- spiro[3.4]octan-6-yl)- benzo[d]thiazole-6- carboxamide | 370.5 | 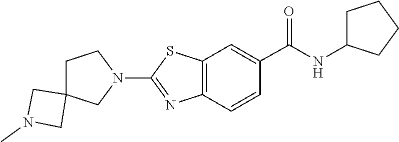 [SMILES: CN1CC2(CCN(c3nc4ccc(C(=O)NC5CCCC5)cc4s3)C2)C1] |
| Example 264 | N-cyclopentyl-2-(2- methyl-2,7-diaza- spiro[3.5]nonan-7-yl)- benzo[d]thiazole-6- carboxamide | 384.5 | 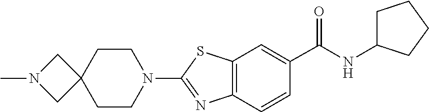 [SMILES: CN1CC2(CCN(c3nc4ccc(C(=O)NC5CCCC5)cc4s3)CC2)C1] |
| Example 265 | N-cyclopentyl-2-(1- methyloctahydro-4H- pyrrolo[3,2-b]pyridin- 4-yl)benzo[d]thiazole- 6-carboxamide | 384.5 | 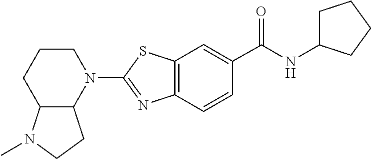 [SMILES: CN1CCC2C1CCCN2c1nc2ccc(C(=O)NC3CCCC3)cc2s1] |
| Example 266 | N-cyclopentyl-2-(1- methyloctahydro-5H- pyrrolo[3,2-c]pyridin-5- yl)benzo[d]thiazole-6- carboxamide | 384.5 | 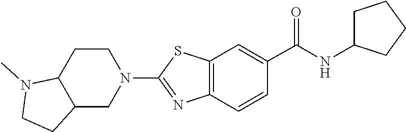 [SMILES: CN1CCC2CN(c3nc4ccc(C(=O)NC5CCCC5)cc4s3)CCC21] |
| Example 267 | (S)-N-(chroman-4-yl)- N-methyl-2-(piperazin- 1-yl)benzo[d]thiazole- 6-carboxamide | 408.5 | 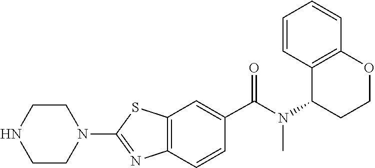 [SMILES: CN(C(=O)c1ccc2nc(N3CCNCC3)sc2c1)[C@H]1CCOc2ccccc21] |
| Example 268 | (S)-N-(chroman-4-yl)- N-methyl-2-(2-methyl- pyridin-3-yl)benzo- [d]thiazole-6-carbox- amide | 415.5 | 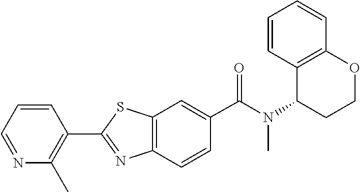 [SMILES: Cc1ncccc1-c1nc2ccc(C(=O)N(C)[C@H]3CCOc4ccccc43)cc2s1] |
| Example 269 | (2-(piperidin-4-yl)- benzo[d]thiazol-6-yl)- (pyrrolidin-1-yl)meth- anone | 315.4 | 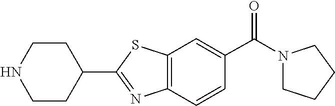 [SMILES: O=C(c1ccc2nc(C3CCNCC3)sc2c1)N1CCCC1] |
| Example 270 | morpholino(2- (piperidin-4-yl)benzo- [d]thiazol-6-yl)meth- anone | 331.4 | 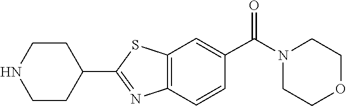 [SMILES: O=C(c1ccc2nc(C3CCNCC3)sc2c1)N1CCOCC1] |
| Example 271 | N-cyclopentyl-N- methyl-2-(piperidin-4- yl)benzo[d]thiazole-6- carboxamide | 343.5 | 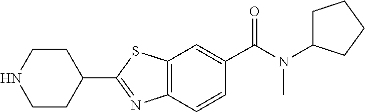 [SMILES: CN(C(=O)c1ccc2nc(C3CCNCC3)sc2c1)C1CCCC1] |
| Example 272 | 2-(piperidin-4-yl)-N- (pyridin-4-yl-methyl)- benzo[d]thiazole-6- carboxamide | 352.5 | 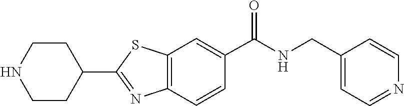 [SMILES: O=C(NCc1ccncc1)c1ccc2nc(C3CCNCC3)sc2c1] |
| Example 273 | 2-(piperidin-4-yl)-N- (pyridin-3-yl-methyl)- benzo[d]thiazole-6- carboxamide | 352.5 | 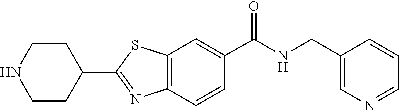 [SMILES: O=C(NCc1cccnc1)c1ccc2nc(C3CCNCC3)sc2c1] |
| Example 274 | N-((1R,2R)-2-hydroxy- cyclopentyl)-2-(1-meth- ylpiperidin-4-yl)benzo- [d]thiazole-6-carbox- amide | 359.5 | 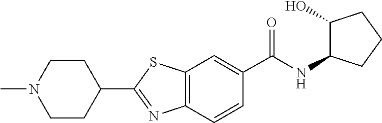 [SMILES: CN1CCC(c2nc3ccc(C(=O)N[C@@H]4CCC[C@H]4O)cc3s2)CC1] |
| Example 275 | 2-(1-ethylpiperdin-4- yl)-N-(pyridin-4-yl- methyl)benzo[d]- thiazole-6-carboxamide | 380.5 | 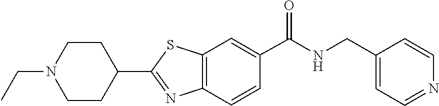 [SMILES: CCN1CCC(c2nc3ccc(C(=O)NCc4ccncc4)cc3s2)CC1] |
| Example 276 | 2-(1-ethylpiperidin-4- yl)-N-(pyridin-3-yl- methyl)benzo[d]thia- zole-6-carboxamide | 380.5 | 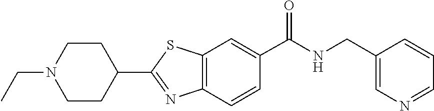 [SMILES: CCN1CCC(c2nc3ccc(C(=O)NCc4cccnc4)cc3s2)CC1] |
| Example 277 | 2-(1-ethylpiperidin-4- yl)-N-(pyridazin-4-yl- methyl)benzo[d]thia- zole-6-carboxamide | 381.5 | 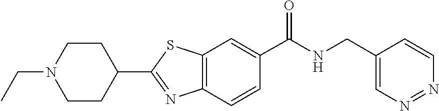 [SMILES: CCN1CCC(c2nc3ccc(C(=O)NCc4ccnnc4)cc3s2)CC1] |
| Example 278 | 2-(3,8-diazabicyclo- [3.2.1]octan-8-yl)-N- (2-isopropoxyethyl)- benzo[d]thiazole-6- carboxamide | 374.5 | 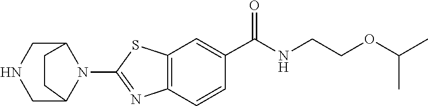 [SMILES: CC(C)OCCNC(=O)c1ccc2nc(N3C4CCC3CNC4)sc2c1] |
| Example 279 | 2-(3,8-diazabicyclo- [3.2.1]octan-8-yl)-N- (2-(cyclopentyloxy)- ethyl)benzo[d]thiazole- 6-carboxamide | 400.5 | 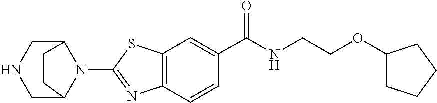 [SMILES: O=C(NCCOC1CCCC1)c1ccc2nc(N3C4CCC3CNC4)sc2c1] |
| Example 280 | N-cyclopentyl-2-(1,2- dimethyl-1H-imidazol- 5-yl)benzo[d]thiazole- 6-carboxamide | 340.4 | 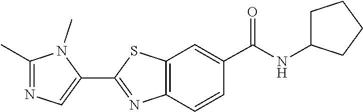 [SMILES: Cc1ncc(-c2nc3ccc(C(=O)NC4CCCC4)cc3s2)n1C] |
| Example 281 | N-cyclopentyl-2-(1- ethylazetidin-3-yl)- benzo[d]thiazole-6- carboxamide | 329.5 | 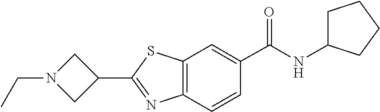 [SMILES: CCN1CC(c2nc3ccc(C(=O)NC4CCCC4)cc3s2)C1] |
| Example 282 | rac-N-cyclopentyl-2- ((3S,4R)-3-hydroxy-1- methylpiperidin-4-yl)- benzo[d]thiazole-6- carboxamide | 359.5 | 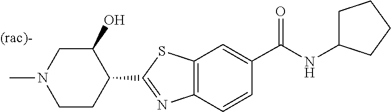 [SMILES: CN1CC[C@@H](c2nc3ccc(C(=O)NC4CCCC4)cc3s2)[C@H](O)C1] |
| Example 283 | N-cyclopentyl-2- (piperidin-4-yl)benzo- [d]thiazole-5-carbox- amide | 329.5 | 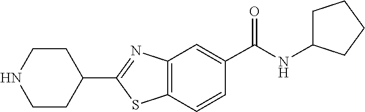 [SMILES: O=C(NC1CCCC1)c1ccc2sc(C3CCNCC3)nc2c1] |
| Example 284 | 2-(3,8-diazabicyclo- [3.2.1]octan-8-yl)-N- cyclopentylbenzo[d]- thiazole-5-carboxamide | 356.5 | 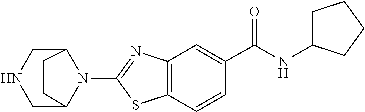 [SMILES: O=C(NC1CCCC1)c1ccc2sc(N3C4CCC3CNC4)nc2c1] |
| Example 285 | N-cyclopentyl-2-(3- methyl-3,8-diazabi- cyclo[3.2.1]octan-8-yl)- benzo[d]thiazole-5- carboxamide | 370.5 | 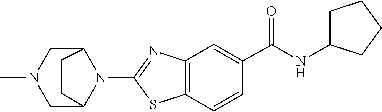 [SMILES: CN1CC2CCC(C1)N2c1nc2cc(C(=O)NC3CCCC3)ccc2s1] |
| Example 286 | N-cyclopentyl-2-(4- ethylpiperazin-1-yl)- benzo[d]thiazole-6- sulfonamide | 394.6 | 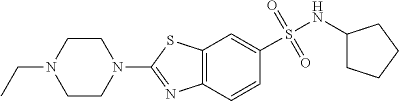 [SMILES: CCN1CCN(c2nc3ccc(S(=O)(=O)NC4CCCC4)cc3s2)CC1] |
| Example 287 | N-cyclopentyl-2- (piperidin-4-yl)benzo- [d]thiazole6-sulfon- amide | 365.5 | 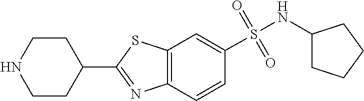 [SMILES: O=S(=O)(NC1CCCC1)c1ccc2nc(C3CCNCC3)sc2c1] |
| Example 288 | N-cyclopentyl-2-(1- methylpiperidin-4-yl)- benzo[d]thiazole-6- sulfonamide | 379.5 | 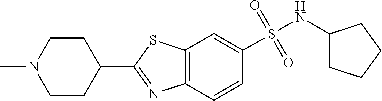 [SMILES: CN1CCC(c2nc3ccc(S(=O)(=O)NC4CCCC4)cc3s2)CC1] |
| Example 289 | 2-(3,8-diazabicyclo- [3.2.1]octan-8-yl)-N- (4,4-difluorocyclohex- yl)benzo[d]thiazole-6- sulfonamide | 442.5 | 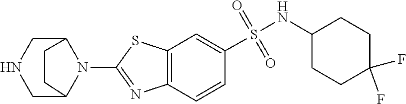 [SMILES: O=S(=O)(NC1CCC(F)(F)CC1)c1ccc2nc(N3C4CCC3CNC4)sc2c1] |
| Example 290 | N-(4,4-difluorocyclo- hexyl)-2-(3-ethyl-3,8- diazabicyclo[3.2.1]- octan-8-yl)benzo[d]- thiazole-6-sulfonamide | 470.6 | 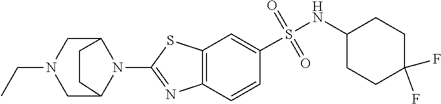 [SMILES: CCN1CC2CCC(C1)N2c1nc2ccc(S(=O)(=O)NC3CCC(F)(F)CC3)cc2s1] |
| Example 291 | N-(4,4-difluorocyclo- hexyl)-2-(piperidin-4- yl)benzo[d]thiazole-6- sulfonamide | 415.5 | 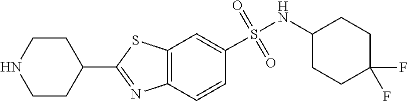 [SMILES: O=S(=O)(NC1CCC(F)(F)CC1)c1ccc2nc(C3CCNCC3)sc2c1] |
| Example 292 | N-(4,4-difluorocyclo- hexyl)-2-(1-(2-hydroxy- ethyl)piperidin-4-yl)- benzo[d]thiazole-6- sulfonamide | 459.6 | 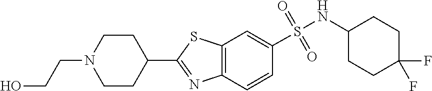 [SMILES: O=S(=O)(NC1CCC(F)(F)CC1)c1ccc2nc(C3CCN(CCO)CC3)sc2c1] |

TABLE 2
Enzyme assay data for PDE4D5, a long form of PDE4
Using the method described in Experiment 1, the
following PDE4D5 activation data were obtained
for exemplary compounds of the present invention.

| Compound Example | Increase in PDE4D5 activity |
|---|---|
| Number | at 30 μM* (%) |
| 1 | 48 |
| 7 | 109 |
| 8 | 82 |
| 9 | 81 |
| 10 | 78 |
| 11 | 104 |
| 12 | 58 |
| 13 | 93 |
| 14 | 120 |
| 15 | 99 |
| 16 | 64 |
| 17 | 171 |
| 18 | 142 |
| 19 | 73 |
| 20 | 105 |
| 21 | 193 |
| 22 | 39 |
| 23 | 94 |
| 24 | 97 |
| 25 | 37 |
| 26 | 88 |
| 27 | 154 |
| 41 | 194 |
| 42 | 138 |
| 44 | 87 |
| 45 | 102 |
| 47 | 102 |
| 48 | 135 |
| 49 | 131 |
| 50 | 51 |
| 51 | 87 |
| 54 | 106 |
| 55 | 49 |
| 56 | 107 |
| 57 | 100 |
| 58 | 101 |
| 59 | 77 |
| 60 | 122 |
| 61 | 124 |
| 62 | 133 |
| 63 | 110 |
| 64 | 64 |
| 66 | 78 |
| 67 | 89 |
| 72 | 32 |
| 76 | 57 |
| 187 | 31 |
| 212 | 30 |
| 216 | 88 |
| 220 | 36 |
| 290 | 47 |

*Measured as mean % increase in counts over basal activity

TABLE 3
Enzyme assay data for PDE4C3, another long form of PDE4
Using the method described in Experiment 1, the
following PDE4C3 activation data were obtained
for exemplary compounds of the present invention.

| Compound Example Number | % increase in PDE4C3 activity at 30 μM* |
|---|---|
| 6 | 38 |
| 7 | 105 |
| 31 | 76 |
| 32 | 99 |
| 33 | 82 |
| 35 | 36 |
| 37 | 57 |
| 38 | 32 |
| 39 | 35 |
| 43 | 68 |
| 46 | 71 |
| 50 | 58 |
| 51 | 112 |
| 53 | 46 |

*Measured as mean % increase in counts over basal activity

TABLE 5
Inhibition of PGE2-stimulated in vitro cyst formation in m-IMCD3 cells
Using the method described in Experiment 4, the following
m-IMCD3 kidney cell cyst suppression data were obtained
for exemplary compounds of the present invention.

| Compound Example | % Cyst area^{†} after incubation |
|---|---|
| Number | with 10 μM test compound |
| 1 | −7 |
| 2 | −9 |
| 3 | 23 |
| 4 | 6 |
| 5 | 1 |
| 6 | 7 |
| 7 | −7 |
| 25 | 23 |
| 28 | 26 |
| 29 | 22 |
| 30 | 21 |
| 32 | 18 |
| 34 | 26 |
| 36 | 30 |
| 47 | 12 |
| 48 | 14 |
| 49 | 9 |
| 50 | 11 |
| 51 | −15 |
| 52 | 24 |
| 55 | 18 |
| 56 | 10 |
| 59 | 20 |
| 61 | 8 |
| 63 | 16 |
| 65 | 25 |
| 69 | 20 |
| 70 | −18 |
| 71 | −1 |
| 73 | 26 |
| 74 | 8 |
| 75 | −1 |
| 78 | 24 |
| 80 | 26 |
| 81 | 7 |
| 82 | 6 |
| 83 | 6 |
| 85 | −12 |
| 88 | 22 |
| 93 | 9 |
| 96 | 2 |
| 98 | 27 |
| 104 | 30 |
| 107 | 17 |
| 108 | 22 |
| 120 | −5 |
| 121 | 15 |
| 124 | 11 |
| 127 | 13 |
| 128 | 3 |
| 130 | −2 |
| 134 | 16 |
| 138 | 20 |
| 151 | 10 |
| 166 | −10 |
| 171 | 13 |
| 175 | −14 |
| 176 | 1 |
| 181 | 13 |
| 186 | 4 |
| 191 | 3 |
| 193 | 2 |
| 196 | 10 |
| 200 | 3 |
| 206 | 14 |
| 210 | 3 |
| 216 | −5 |
| 219 | 2 |
| 222 | 1 |
| 224 | 8 |
| 230 | 25 |
| 231 | 1 |
| 232 | −2 |
| 234 | 3 |
| 237 | 11 |
| 240 | 5 |
| 243 | 6 |
| 249 | 9 |
| 255 | 11 |
| 258 | 11 |
| 261 | 3 |
| 271 | 27 |
| 274 | 16 |
| 275 | −2 |
| 278 | 18 |
| 281 | 10 |
| 283 | 14 |
| 288 | 11 |
| 290 | 16 |

^{†}Mean cyst area (%) compared to 100% for (DMSO + PGE2) and 0% for DMSO control

TABLE 4
Enzyme assay data for PDE4B2, a short form of PDE4
Using the method described in Experiment 1, the following PDE4B2
data were obtained for exemplary compounds of the present invention.

| Compound Example Number | % increase in PDE4B2 activity at 30 μM* |
|---|---|
| 9 | 3 |
| 187 | 9 |
| 212 | −3 |
| 216 | −2 |
| 220 | 0 |
| 290 | 11 |

*Measured as mean % increase in counts over basal activity

TABLE 6
Inhibition of PGE2-stimulated in vitro cyst formation in MDCK cells
Using the method described in Experiment 3, the following
MDCK kidney cell cyst suppression data were obtained
for exemplary compounds of the present invention.

| Compound Example | % Cyst area^{‡} after incubation |
|---|---|
| Number | with 1 μM test compound |
| 83 | −1 |
| 236 | 20 |
| 238 | 24 |
| 245 | 4 |
| 247 | 1 |
| 260 | 11 |

^{‡}Total cyst area (%) compared to 100% for (DMSO + PGE2) and 0% for DMSO control

TABLE 7
Reduction of cAMP levels in m-IMCD3 cell culture
Using the method described in Experiment 4a, the following
m-IMCD3 cell culture cAMP measurements were obtained
for exemplary compounds of the present invention.

| Compound Example | cAMP level (% of PGE2-stimulated) after |
|---|---|
| Number | incubation with 10 μM test compound^{§} |
| 151 | 54 |
| 174 | 27 |
| 240 | 41 |

^{§}Compared to 100% for (DMSO + PGE2)

It will be appreciated that the above description is made by way of example and not limitation of the scope of the appended claims, including any equivalents as included within the scope of the claims. Various modifications are possible and will be readily apparent to the skilled person in the art. Likewise, features of the described embodiments can be combined with any appropriate aspect described above and optional features of any one aspect can be combined with any other appropriate aspect.

1. A method for treating or preventing of a disease or disorder that can be ameliorated by activation of long isoforms of PDE4, comprising administering to a patient in need thereof a therapeutically effective amount of a compound of Formula A: 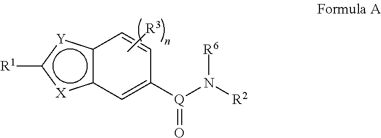
or a pharmaceutically acceptable salt or derivative thereof, wherein:
one of X and Y is S and the other is N;
Q is C or S(O);
R¹ is a 4- to 10-membered monocyclic, bridged or bicyclic ring containing at least 1 ring N heteroatom and optionally a ring 0 heteroatom, and wherein R¹ is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring 0 heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof, wherein a straight chain portion of said (C3-8)alkyl group may be optionally interrupted by 1—O—;
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
R⁶ is H or (C1-6)alkyl; and
n is 0, 1, 2 or 3.

2. The method of claim 1, where in the compound is a compound of Formula I: 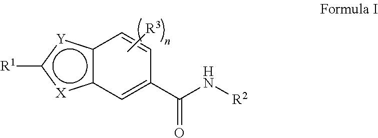
or a pharmaceutically acceptable salt or derivative thereof, wherein:
one of X and Y is S and the other is N;
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof;
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and
n is 0, 1, 2 or 3.

3. The method of claim 1, wherein R¹ is a 5- to 6-membered saturated, monocyclic ring containing at least 1 ring N heteroatom and optionally a ring O heteroatom; a 5 to 6-membered aromatic, monocyclic ring containing 1 or 2 ring N heteroatoms; a 7- to 8-membered saturated, bridged ring system containing 1 or 2 ring N heteroatoms; a 9-membered saturated, bridged ring system containing 2 ring N heteroatoms and a ring O-heteroatom; or a 7 to 10-membered saturated, fused or spiro ring system containing 1 or 2 ring N heteroatoms; and wherein R¹ is optionally substituted with 1, 2 or 3 R⁴.

4. The method of claim 1, wherein R¹ is a 6-membered saturated or aromatic monocyclic ring containing 2 ring N heteroatoms; or a 7- to 8-membered saturated, bridged ring system containing 2 ring N heteroatoms; wherein R¹ is optionally substituted with 1 R⁴.

5. The method of claim 1, wherein R¹ is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

6. The method of claim 1, wherein when Q is S(O), R is not optionally substituted pyrazol-4-yl.

7. The method of claim 1, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein the Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, (C1-4)alkoxy or OH.

8. The method of claim 1, wherein R² is:
(i) (C5-6)cycloalkyl fused to a phenyl ring;
(ii) a 5- to 6-membered non-aromatic heterocycle containing one ring O heteroatom, optionally fused to a phenyl ring; or
(iii) (C4-6)cycloalkyl;
wherein R² is optionally substituted with 1 or more R⁵.

9. The method of claim 1, wherein R² is a group of formula 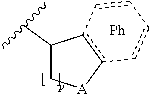
wherein A is O or CH₂; p is 1, 2 or 3; Ph is an optionally present, fused phenyl ring, and wherein R² is optionally substituted with 1 or more R⁵; optionally wherein A is O or C(R⁵)₂ (for example, CF₂).

10. The method of claim 1, wherein each R³ is independently —CH₃, —OCH₃, halo, CN or cyclopropyl.

11. The method of claim 1, wherein n is 0, 1 or 2, preferably 0 or 1.

12. The method of claim 1, wherein the compound is: 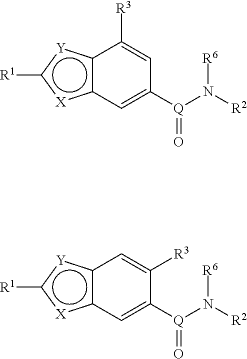
or a pharmaceutically acceptable salt or derivative thereof.

13. The method of claim 1, wherein X is S and Y is N.

14. The method of claim 1, wherein
X is S and Y is N;
R¹ is a 6-membered saturated or aromatic monocyclic ring containing 2 ring N heteroatoms or a 7- to 8-membered saturated, bridged ring system containing 2 ring N heteroatoms, wherein R¹ is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 or 2 R⁵;
R³, where present, is methyl, CN or halogen;
R⁴, where present, is (C1-6)alkyl optionally substituted with OH, optionally (C1-2)alkyl optionally substituted with OH;
R⁵, where present, is OH or halo; and
n is 0 or 1.

15. A compound of Formula B: 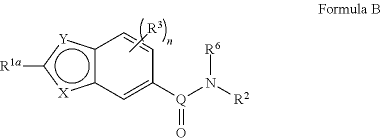
or a pharmaceutically acceptable salt or derivative thereof, wherein:
one of X and Y is S and the other is N;
Q is C or S(O);
R^{1a} is a 4- to 10-membered non-aromatic, monocyclic, bridged or bicyclic ring containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof, wherein a straight chain portion of said (C3-8)alkyl group may be optionally interrupted by 1-O—, optionally a (C4-8)alkyl group that may be straight chain, branched or cyclic, or a combination thereof, wherein a straight chain portion of said (C4-8)alkyl group may be optionally interrupted by 1—O—;
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH;
R⁶ is H or (C1-6)alkyl; and
n is 0, 1, 2 or 3;
wherein when R^{1a} is 4-cyclopentylpiperazin-1-yl, 4-cyclopropylpiperazin-1-yl or 4-isopropylpiperazin-1-yl, Q is C and n is 0, R² is not unsubstituted, uninterrupted, straight chain or branched (C3-6)alkyl or unsubstituted (C3-8)cycloalkyl; and
wherein the compound is not 2-(1-piperazinyl)-N-propyl-6-benzothiazolecarboxamide, N-(1-methylethyl)-2-(1-piperazinyl)-6-benzothiazolecarboxamide or N-cyclopropyl-2-(1-piperazinyl)-6-benzothiazolecarboxamide.

16. The compound of claim 15, wherein the compound is a compound of Formula II 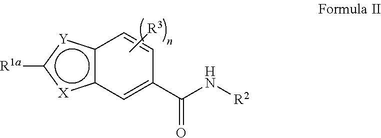
or a pharmaceutically acceptable salt or derivative thereof, wherein:
one of X and Y is S and the other is N;
R^{1a} is a 4- to 10-membered non-aromatic, monocyclic, bridged or bicyclic ring containing at least 1 ring N heteroatom and optionally a ring O heteroatom, wherein at least 1 ring N heteroatom is not at the point of attachment of R^{1a}, and wherein R^{1a} is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof, optionally a (C4-8)alkyl group that may be straight chain, branched or cyclic, or a combination thereof,
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy, the (C1-6)alkyl and (C1-6)alkoxy being optionally substituted by 1 or more halogen or OH; and
n is 0, 1, 2 or 3;
wherein when R^{1a} is 4-cyclopentylpiperazin-1-yl, 4-cyclopropylpiperazin-1-yl or 4-isopropylpiperazin-1-yl and n is 0, R² is not unsubstituted, straight chain or branched (C3-6)alkyl or unsubstituted (C3-8)cycloalkyl.

17. The compound or a pharmaceutically acceptable salt or derivative thereof claim 15, wherein:
a) each R⁴ is independently halogen, CN, OH, (C1-2)alkyl, (C1-6)alkoxy or —(C1-6)alkylene-(C1-6)alkoxy, the (C1-2)alkyl, (C1-6)alkoxy and —(C1-6)alkylene-(C1-6)alkoxy being optionally substituted with 1 or more substituents independently selected from halogen, OH and (C1-6)alkoxy; and/or
b) n is 1, 2 or 3; and/or
c) R² is (C5-7)cycloalkyl, fused to a 6-membered aromatic or heteroaromatic ring that contains 0, 1 or 2 ring N atoms; a 5- to 7-membered non-aromatic heterocycle containing one ring O heteroatom, optionally fused to a 6-membered aromatic or heteroaromatic ring that contains 0, 1 or 2 ring N atoms; or CH₂Ar, where Ar is a 6-membered aromatic or heteroaromatic ring that contains 0, 1 or 2 ring N atoms; and wherein R² is optionally substituted with 1 or more R⁵.

18. The compound or a pharmaceutically acceptable salt or derivative thereof claim 15, wherein R^{1a} is a 5- to 6-membered saturated, monocyclic ring containing at least 1 ring N heteroatom and optionally a ring O heteroatom; or a 7- to 8-membered saturated, bridged ring system containing 1 or 2 ring N heteroatoms; a 9-membered saturated, bridged ring system containing 2 ring N heteroatoms and a ring O-heteroatom; or a 7 to 10-membered saturated, fused or spiro ring system containing 1 or 2 ring N heteroatoms; and wherein R^{1a} is optionally substituted with 1, 2 or 3 R⁴.

19. The compound, or a pharmaceutically acceptable salt or derivative thereof, of claim 15, wherein R^{1a} is a 6-membered saturated monocyclic ring containing 2 ring N heteroatoms or a 7- to 8-membered saturated, bridged ring system containing 2 ring N heteroatoms; wherein R^{1a} is optionally substituted with 1 R⁴, optionally wherein R^{1a} is a 7- to 8-membered saturated, bridged ring system containing 2 ring N heteroatoms (for example a bridged piperazine, such as 3,8-diazabicyclo[3.2.1]octanyl), wherein R^{1a} is optionally substituted with 1 R⁴.

20. The compound, or a pharmaceutically acceptable salt or derivative thereof, of claim 15, wherein R² is:
(i) (C5-7)cycloalkyl, fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered heterocycle is optionally substituted with 1 to 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(iii) CH₂Ar, wherein Ar is optionally substituted with 1 to 3 substituents selected from halogen, CN, (C1-4)alkyl, (C1-4)alkoxy and the CH₂ is optionally substituted with (C1-4)alkyl the (C1-4)alkyl group being optionally substituted with OH or (C1-4)alkyloxy; or
(iv) a (C3-8)alkyl group that may be straight chain, branched or cyclic or a combination thereof, optionally substituted with 1 or more halogen, OH or (C1-4)alkoxy, optionally a (C4-8)alkyl group that may be straight chain, branched or cyclic or a combination thereof, optionally substituted with 1 or more halogen, OH or (C1-4)alkoxy.

21. The compound, or a pharmaceutically acceptable salt or derivative thereof, of claim 15, wherein R² is:
(i) (C5-6)cycloalkyl fused to a phenyl ring;
(ii) a 5- to 6-membered non-aromatic heterocycle containing one ring O heteroatom, optionally fused to a phenyl ring; or
(iii) (C4-6)cycloalkyl;
wherein R² is optionally substituted with 1 or more R⁵.

22. The compound, or a pharmaceutically acceptable salt or derivative thereof, of claim 15, wherein R² is a group of formula 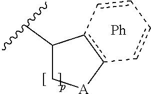
wherein A is O or CH₂; p is 1, 2 or 3; Ph is an optionally present, fused phenyl ring, and wherein R² is optionally substituted with 1 or more R⁵; optionally wherein A is O or C(R⁵)₂ (for example, CF₂).

23. The compound, or a pharmaceutically acceptable salt or derivative thereof, of claim 15, wherein
X is Sand Y is N;
R^{1a} is a 6-membered saturated monocyclic ring containing 2 ring N heteroatoms or a 7- to 8-membered saturated, bridged ring system containing 2 ring N heteroatoms, wherein R^{1a} is optionally substituted with 1 R⁴;
R² is
(i) (C5-6)cycloalkyl, optionally fused to a phenyl ring; or
(ii) a 5- to 6-membered non-aromatic heterocycle containing one ring O heteroatom, optionally fused to a phenyl ring;
and wherein R² is optionally substituted with 1 or 2 R⁵;
R³, where present, is methyl, CN or halogen;
R⁴, where present, is (C1-6)alkyl optionally substituted with OH, optionally (C1-2)alkyl optionally substituted with OH;
R⁵, where present, is OH or halo; and
n is 0 or 1.

24. A compound of Formula C: 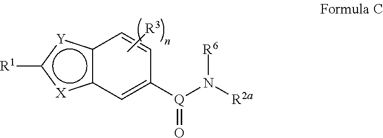
or a pharmaceutically acceptable salt or derivative thereof, wherein:
one of X and Y is S and the other is N;
Q is C or S(O);
R¹ is a 4- to 10-membered monocyclic, bridged or bicyclic ring containing at least 1 ring N heteroatom and optionally a ring O heteroatom (optionally a 4- to 10-membered monocyclic, bridged or bicyclic ring containing at least 1 ring N heteroatom), and wherein R¹ is optionally substituted with 1 or more R⁴;
R^{2a} is
(i) (C5-7)cycloalkyl fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iv) a (C4-6)cycloalkyl group;
and wherein R^{2a} is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH;
R⁶ is H or (C1-6)alkyl; and
n is 0, 1, 2 or 3; and
wherein when R^{2a} is (iv) a (C4-6)cycloalkyl group it is substituted by at least 2 R⁵; and
wherein the compound is not 2-(4-morpholinyl)-N-(1,2,3,4-tetrahydronaphthalenyl)-6-benzothiazolcarboxamide or N-(2,3-dihydro-1H-inden-2-yl)-2−(1H-pyrrol-1-yl)-6-benzothiazolecarboxamide.

25. The compound of claim 24, wherein the compound is a compound of Formula III: 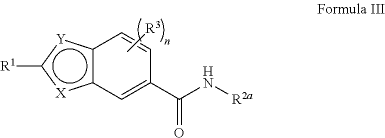
or a pharmaceutically acceptable salt or derivative thereof, wherein:
one of X and Y is S and the other is N;
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R^{2a} is
(i) (C5-7)cycloalkyl fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iv) a (C4-6)cycloalkyl group;
and wherein R^{2a} is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy;
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and
n is 0, 1, 2 or 3; and
wherein when R^{2a} is (iv) a (C4-6)cycloalkyl group it is substituted by at least 2 R⁵.

26. The compound, or a pharmaceutically acceptable salt or derivative thereof, of claim 15, wherein n is 0, 1 or 2, optionally n is 0 or 1.

27. A compound of Formula D: 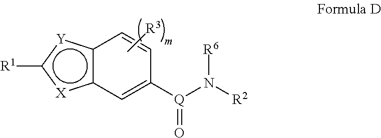
or a pharmaceutically acceptable salt or derivative thereof, wherein:
one of X and Y is S and the other is N;
Q is C or S(O);
R¹ is a 4- to 10-membered monocyclic, bridged or bicyclic ring containing at least 1 ring N heteroatom and optionally a ring 0 heteroatom, and wherein R¹ is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring 0 heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof, wherein a straight chain portion of said (C3-8)alkyl group may be optionally interrupted by 1—O—;
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen, OH or (C1-4)alkoxy;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH;
R⁶ is H or (C1-6)alkyl; and
m is 1, 2 or 3; and
wherein when Q is S(O), R′ is not optionally substituted pyrazol-4-yl.

28. A compound of Formula IV 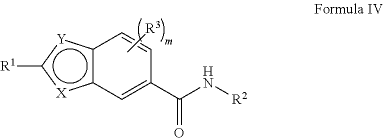
or a pharmaceutically acceptable salt or derivative thereof, wherein:
one of X and Y is S and the other is N;
R¹ is a 4- to 10-membered monocyclic, bridged or bicyclic ring containing at least 1 ring N heteroatom and optionally a ring O heteroatom, and wherein R¹ is optionally substituted with 1 or more R⁴;
R² is
(i) (C5-7)cycloalkyl, fused to a 6-membered aromatic or heteroaromatic ring that contains 0, 1 or 2 ring N atoms;
(ii) a 5- to 7-membered non-aromatic heterocycle containing one ring O heteroatom, optionally fused to a 6-membered aromatic or heteroaromatic ring that contains 0, 1 or 2 ring N atoms;
(iii) CH₂Ar, where Ar is a 6-membered aromatic or heteroaromatic ring that contains 0, 1 or 2 ring N atoms; or
(iv) a (C3-8)alkyl group that may be straight chain, branched or cyclic, or a combination thereof,
and wherein R² is optionally substituted with 1 or more R⁵;
each R³ is independently (C1-6)alkyl, (C1-6)alkoxy, CN or halogen, the (C1-6)alkyl and (C1-6)alkoxy being optionally substituted by 1 or more halogen;
each R⁴ is independently halogen, CN, OH, (C1-6)alkyl, (C1-6)alkoxy, (C3-7)cycloalkyl or —(C1-6)alkylene-(C1-6)alkoxy, the (C1-6)alkyl, (C1-6)alkoxy, (C3-7)cycloalkyl and —(C1-6)alkylene-(C1-6)alkoxy being optionally substituted with 1 or more substituents independently selected from halogen, OH and (C1-6)alkoxy; and
each R⁵ is independently halogen, OH, CN, (C1-6)alkyl, (C1-6)alkoxy or —(C1-6)alkylene-(C1-6)alkoxy the (C1-6)alkyl and (C1-6)alkoxy being optionally substituted by 1 or more halogen or OH; and
m is 1,2 or 3.

29. The compound, or a pharmaceutically acceptable salt or derivative thereof, of claim 28, wherein the compound is a compound of
a) Formula D and the compound is a compound of Formula D′ or D″ 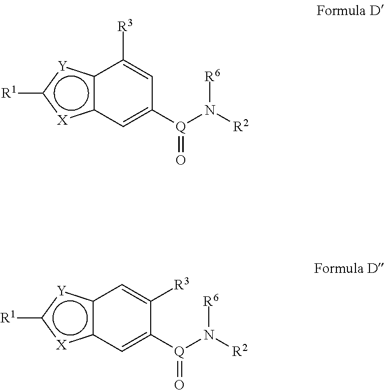
or a pharmaceutically acceptable salt or derivative thereof, optionally wherein the compound is a compound of Formula D′; or
b) Formula IV and the compound is a compound of Formula IVa or IVb 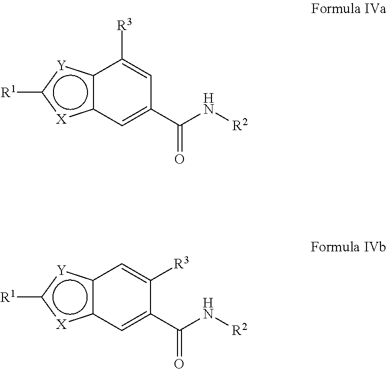
or a pharmaceutically acceptable salt or derivative thereof.

30. The compound, or a pharmaceutically acceptable salt or derivative thereof, of claim 15, wherein each R³ is independently —CH₃, —OCH₃, halo, CN or cyclopropyl.

31. The compound, or a pharmaceutically acceptable salt or derivative thereof, of claim 15, wherein R² or R^{2a} is:
(i) (C5-7)cycloalkyl fused to a 6-membered aromatic or heteroaromatic ring that contains 0, 1 or 2 ring N atoms, wherein the (C5-7)cycloalkyl is optionally substituted with 1 to 3 substituents independently selected from OH, halogen, (C1-4)alkyl, (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro;
(ii) a 5- to 7-membered non-aromatic heterocycle containing one ring O heteroatom, optionally fused to a 6-membered aromatic or heteroaromatic ring that contains 0, 1 or 2 ring N atoms, wherein the 5- to 7-membered non-aromatic heterocycle is optionally substituted with 1 to 3 substituents on one or more ring carbon atoms independently selected from OH, halogen, (C1-4)alkyl, and (C1-4)alkoxy, the (C1-4)alkyl and (C1-4)alkoxy groups being optionally substituted with one or more fluoro, and the 6-membered aromatic or heteroaromatic ring is optionally substituted with 1 to 3 substituents independently selected from (C1-4)alkyl, (C1-4)alkoxy, CN and halogen, the (C1-4)alkyl and (C1-4)alkoxy groups being optionally substituted with one or more fluoro; or
(iv) a (C5-6)cycloalkyl group, substituted by 2 or 3 substituents on one or more ring carbon atoms independently selected from OH, halogen, (C1-4)alkyl, (C1-4)alkoxy, the (C1-4)alkyl and (C1-4)alkoxy groups being optionally substituted with one or more fluoro, optionally wherein the (C5-6)cycloalkyl group is substituted by 2 halogen substituents (optionally on a single ring carbon atom).

32. The compound, or a pharmaceutically acceptable salt or derivative thereof, of claim 15, wherein R² or R^{2a} is:
(i) (C5-6)cycloalkyl fused to a phenyl ring;
(ii) a 5- to 6-membered non-aromatic heterocycle containing one ring O heteroatom, optionally fused to a phenyl ring; or
(iv) a (C4-6)cycloalkyl group;
wherein R² or R^{2a} is optionally substituted, wherein when R^{2a} is (iv) a (C4-6)cycloalkyl group it is substituted by at least 2 R⁵.

33. The compound, or a pharmaceutically acceptable salt or derivative thereof, of claim 15, wherein R² or R^{2a} is a group of formula 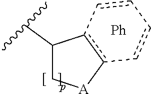
wherein A is O or CH₂; p is 1, 2 or 3; Ph is an optionally present, fused phenyl ring, and wherein R² or R^{2a} is optionally substituted with 1 or more R⁵; and wherein when A is CH₂, Ph is present or A is C(R⁵)₂ (for example, CF₂).

34. The compound, or a pharmaceutically acceptable salt or derivative thereof, of claim 24, wherein
X is Sand Y is N;
R¹ is a 6-membered saturated or aromatic monocyclic ring containing 2 ring N heteroatoms or a 7- to 8-membered saturated, bridged ring system containing 2 ring N heteroatoms, wherein R¹ is optionally substituted with 1 R⁴;
R^{2a} is
(i) (C5-6)cycloalkyl fused to a phenyl ring; or
(ii) a 5- to 6-membered non-aromatic heterocycle containing one ring O heteroatom, optionally fused to a phenyl ring; or
(iv) a (C4-6)cycloalkyl group;
and wherein R^{2a} is optionally substituted with 1 or 2 R⁵, wherein when R^{2a} is (iv) a (C4-6)cycloalkyl group it is substituted by 2 R⁵;
R⁴, where present, is (C1-6)alkyl optionally substituted with OH, optionally (C1-2)alkyl optionally substituted with OH;
R⁵, where present, is OH or halo; and
n is 0 or 1.

35. The compound or a pharmaceutically acceptable salt or derivative thereof, of claim 24, wherein R¹ is a 5- to 6-membered saturated, monocyclic ring containing at least 1 ring N heteroatom and optionally a ring O heteroatom; a 5 to 6-membered aromatic, monocyclic ring containing 1 or 2 ring N heteroatoms; a 7- to 8-membered saturated, bridged ring system containing 1 or 2 ring N heteroatoms; a 9-membered saturated, bridged ring system containing 2 ring N heteroatoms and a ring O-heteroatom; or a 7 to 10-membered saturated, fused or spiro ring system containing 1 or 2 ring N heteroatoms; and wherein R¹ is optionally substituted with 1, 2 or 3 R⁴.

36. The compound, or a pharmaceutically acceptable salt or derivative thereof, of claim 24, wherein R¹ is a 6-membered saturated or aromatic monocyclic ring containing 2 ring N heteroatoms or a 7- to 8-membered saturated, bridged ring system containing 2 ring N heteroatoms, wherein R¹ is optionally substituted with 1 R⁴.

37. The compound, or a pharmaceutically acceptable salt or derivative thereof, of claim 24, wherein R¹ is a 7- to 8-membered saturated, bridged ring system containing 2 ring N heteroatoms (for example a bridged piperazine, such as 3,8-diazabicyclo[3.2.1]octanyl), wherein R¹ is optionally substituted with 1 R⁴.

38. The compound, or a pharmaceutically acceptable salt or derivative thereof, of claim 15, wherein X is S and Y is N.

39. A compound selected from:
N-(4-chlorobenzyl)-2-(2-methylpyridin-3-yl)benzo[d]thiazole-6-carboxamide;
N-(4-fluorobenzyl)-2-(2-methylpyridin-3-yl)benzo[d]thiazole-6-carboxamide;
N-(4-methoxybenzyl)-2-(2-methylpyridin-3-yl)benzo[d]thiazole-6-carboxamide;
N-(3-fluorobenzyl)-2-(2-methylpyridin-3-yl)benzo[d]thiazole-6-carboxamide;
(S)-N-(2,3-dihydro-1H-inden-1-yl)-2-(2-methylpyridin-3-yl)benzo[d]thiazole-6-carboxamide;
(S)-2-(2-methylpyridin-3-yl)-N-(1,2,3,4-tetrahydronaphthalen-1-yl)benzo[d]thiazole-6-carboxamide;
(S)-N-(chroman-4-yl)-2-(2-methylpyridin-3-yl)benzo[d]thiazole-6-carboxamide;
(S)-2-(pyridin-3-yl)-N-(1,2,3,4-tetrahydronaphthalen-1-yl)benzo[d]thiazole-6-carboxamide;
(S)-N-(chroman-4-yl)-2-(pyridin-3-yl)benzo[d]thiazole-6-carboxamide;
N-benzyl-2-(6-methylpyridin-3-yl)benzo[d]thiazole-6-carboxamide;
N-(4-chlorobenzyl)-2-(6-methylpyridin-3-yl)benzo[d]thiazole-6-carboxamide;
N-(4-fluorobenzyl)-2-(6-methylpyridin-3-yl)benzo[d]thiazole-6-carboxamide;
N-(4-methoxybenzyl)-2-(6-methylpyridin-3-yl)benzo[d]thiazole-6-carboxamide;
N-(3-chlorobenzyl)-2-(6-methylpyridin-3-yl)benzo[d]thiazole-6-carboxamide;
N-(3-fluorobenzyl)-2-(6-methylpyridin-3-yl)benzo[d]thiazole-6-carboxamide;
N-(3-methoxybenzyl)-2-(6-methylpyridin-3-yl)benzo[d]thiazole-6-carboxamide;
(S)-N-(2,3-dihydro-1H-inden-1-yl)-2-(6-methylpyridin-3-yl)benzo[d]thiazole-6-carboxamide;
(R)-N-(2,3-dihydro-1H-inden-1-yl)-2-(6-methylpyridin-3-yl)benzo[d]thiazole-6-carboxamide;
N-((1S,2R)-2-hydroxy-2,3-dihydro-1H-inden-1-yl)-2-(6-methylpyridin-3-yl)benzo[d]thiazole-6-carboxamide;
N-((1R,2S)-2-hydroxy-2,3-dihydro-1H-inden-1-yl)-2-(6-methylpyridin-3-yl)benzo[d]thiazole-6-carboxamide;
N-((1R,2R)-2-hydroxy-2,3-dihydro-1H-inden-1-yl)-2-(6-methylpyridin-3-yl)benzo[d]thiazole-6-carboxamide;
N-cyclopentyl-2-(6-methylpyridin-3-yl)benzo[d]thiazole-6-carboxamide;
(S)-2-(6-methylpyridin-3-yl)-N-(1,2,3,4-tetrahydronaphthalen-1-yl)benzo[d]thiazole-6-carboxamide;
(S)-N-(chroman-4-yl)-2-(6-methylpyridin-3-yl)benzo[d]thiazole-6-carboxamide;
(S)-N-(chroman-4-yl)-2-(6-(trifluoromethyl)pyridin-3-yl)benzo[d]thiazole-6-carboxamide;
(S)-N-(chroman-4-yl)-2-(2,6-dimethylpyridin-3-yl)benzo[d]thiazole-6-carboxamide;
(S)-N-(chroman-4-yl)-2-(6-cyclopropylpyridin-3-yl)benzo[d]thiazole-6-carboxamide;
(S)-N-(chroman-4-yl)-2-(6-isopropylpyridin-3-yl)benzo[d]thiazole-6-carboxamide;
(S)-N-(chroman-4-yl)-2-(6-ethylpyridin-3-yl)benzo[d]thiazole-6-carboxamide;
N-benzyl-2-(5-methylpyridin-3-yl)benzo[d]thiazole-6-carboxamide;
N-(4-chlorobenzyl)-2-(5-methylpyridin-3-yl)benzo[d]thiazole-6-carboxamide;
N-(4-fluorobenzyl)-2-(5-methylpyridin-3-yl)benzo[d]thiazole-6-carboxamide;
N-(4-methoxybenzyl)-2-(5-methylpyridin-3-yl)benzo[d]thiazole-6-carboxamide;
N-(3-chlorobenzyl)-2-(5-methylpyridin-3-yl)benzo[d]thiazole-6-carboxamide;
N-(3-fluorobenzyl)-2-(5-methylpyridin-3-yl)benzo[d]thiazole-6-carboxamide;
N-(3-methoxybenzyl)-2-(5-methylpyridin-3-yl)benzo[d]thiazole-6-carboxamide;
(S)-N-(2,3-dihydro-1H-inden-1-yl)-2-(5-methylpyridin-3-yl)benzo[d]thiazole-6-carboxamide;
(R)-N-(2,3-dihydro-1H-inden-1-yl)-2-(5-methylpyridin-3-yl)benzo[d]thiazole-6-carboxamide;
N-((1R,2S)-2-hydroxy-2,3-dihydro-1H-inden-1-yl)-2-(5-methylpyridin-3-yl)benzo[d]thiazole-6-carboxamide;
N-((1S,2R)-2-hydroxy-2,3-dihydro-1H-inden-1-yl)-2-(5-methylpyridin-3-yl)benzo[d]thiazole-6-carboxamide;
N-((1S,2S)-2-hydroxy-2,3-dihydro-1H-inden-1-yl)-2-(5-methylpyridin-3-yl)benzo[d]thiazole-6-carboxamide;
N-((1R,2R)-2-hydroxy-2,3-dihydro-1H-inden-1-yl)-2-(5-methylpyridin-3-yl)benzo[d]thiazole-6-carboxamide;
N-cyclopentyl-2-(5-methylpyridin-3-yl)benzo[d]thiazole-6-carboxamide;
(S)-2-(5-methylpyridin-3-yl)-N-(1,2,3,4-tetrahydronaphthalen-1-yl)benzo[d]thiazole-6-carboxamide;
(S)-N-(chroman-4-yl)-2-(5-methylpyridin-3-yl)benzo[d]thiazole-6-carboxamide;
2-(5-methylpyridin-3-yl)-N-(tetrahydro-2H-pyran-4-yl)benzo[d]thiazole-6-carboxamide;
(S)-N-(chroman-4-yl)-2-(5-(trifluoromethyl)pyridin-3-yl)benzo[d]thiazole-6-carboxamide;
(S)-N-(chroman-4-yl)-2-(5-cyclopropylpyridin-3-yl)benzo[d]thiazole-6-carboxamide;
(S)-N-(chroman-4-yl)-2-(5-isopropylpyridin-3-yl)benzo[d]thiazole-6-carboxamide;
(S)-2-(4-methylpyridin-3-yl)-N-(1,2,3,4-tetrahydronaphthalen-1-yl)benzo[d]thiazole-6-carboxamide;
(S)-N-(chroman-4-yl)-2-(4-methylpyridin-3-yl)benzo[d]thiazole-6-carboxamide;
(S)-N-(chroman-4-yl)-2-(2,4-dimethylpyridin-3-yl)benzo[d]thiazole-6-carboxamide;
(S)-2-(5-chloropyridin-3-yl)-N-(chroman-4-yl)benzo[d]thiazole-6-carboxamide;
(S)-N-(chroman-4-yl)-2-(pyridin-4-yl)benzo[d]thiazole-6-carboxamide;
(S)-N-(chroman-4-yl)-2-(3-methylpyridin-4-yl)benzo[d]thiazole-6-carboxamide;
(S)-N-(chroman-4-yl)-2-(2-methylpyridin-4-yl)benzo[d]thiazole-6-carboxamide;
(S)-N-(chroman-4-yl)-2-(1-methyl-1H-pyrazol-4-yl)benzo[d]thiazole-6-carboxamide;
(S)-N-(chroman-4-yl)-2-(1,4-dimethyl-1H-pyrazol-5-yl)benzo[d]thiazole-6-carboxamide;
(S)-N-(chroman-4-yl)-2-(1,3,5-trimethyl-1H-pyrazol-4-yl)benzo[d]thiazole-6-carboxamide;
(S)-N-(chroman-4-yl)-2-(1,5-dimethyl-1H-pyrazol-4-yl)benzo[d]thiazole-6-carboxamide;
(S)-N-(chroman-4-yl)-2-(1,3-dimethyl-1H-pyrazol-4-yl)benzo[d]thiazole-6-carboxamide;
(S)-N-(chroman-4-yl)-2-(5-(hydroxymethyl)pyridin-3-yl)benzo[d]thiazole-6-carboxamide;
(S)-N-(chroman-4-yl)-2-(5-(difluoromethyl)pyridin-3-yl)benzo[d]thiazole-6-carboxamide;
(S)-N-(chroman-4-yl)-2-(6-(difluoromethyl)pyridin-3-yl)benzo[d]thiazole-6-carboxamide;
(S)-N-(chroman-4-yl)-2-(2-methylpyridin-3-yl)benzo[d]thiazole-5-carboxamide;
(S)-N-(chroman-4-yl)-2-(4-methylpyridin-3-yl)benzo[d]thiazole-5-carboxamide;
(S)-N-(chroman-4-yl)-2-(pyridin-3-yl)benzo[d]thiazole-5-carboxamide;
(S)-N-(chroman-4-yl)-2-(4-hydroxypiperidin-1-yl)benzo[d]thiazole-6-carboxamide;
(S)-N-(chroman-4-yl)-2-morpholinobenzo[d]thiazole-6-carboxamide;
(S)-N-(chroman-4-yl)-2-(4-methoxypiperidin-1-yl)benzo[d]thiazole-6-carboxamide;
(S)-N-(chroman-4-yl)-2-(4-methylpiperazin-1-yl)benzo[d]thiazole-6-carboxamide;
(S)-N-(chroman-4-yl)-2-(4-ethylpiperazin-1-yl)benzo[d]thiazole-6-carboxamide;
(R)-N-(chroman-4-yl)-2-(4-ethylpiperazin-1-yl)benzo[d]thiazole-6-carboxamide;
(S)-N-(chroman-4-yl)-2-(4-isopropylpiperazin-1-yl)benzo[d]thiazole-6-carboxamide;
(S)-2-(4-(tert-butyl)piperazin-1-yl)-N-(chroman-4-yl)benzo[d]thiazole-6-carboxamide;
(S)-N-(chroman-4-yl)-2-(piperidin-1-yl)benzo[d]thiazole-6-carboxamide;
2-((1S,4S)-2,5-diazabicyclo[2.2.1]heptan-2-yl)-N-((S)-chroman-4-yl)benzo[d]thiazole-6-carboxamide;
2-((1R,4R)-2,5-diazabicyclo[2.2.1]heptan-2-yl)-N-((S)-chroman-4-yl)benzo[d]thiazole-6-carboxamide;
2-(2,5-diazabicyclo[2.2.2]octan-2-yl)-N-((S)-chroman-4-yl)benzo[d]thiazole-6-carboxamide;
2-(4-ethylpiperazin-1-yl)-N-(4-fluorobenzyl)benzo[d]thiazole-6-carboxamide;
N-cyclopentyl-2-(4-ethylpiperazin-1-yl)benzo[d]thiazole-6-carboxamide;
2-(4-ethylpiperazin-1-yl)-N-isopropylbenzo[d]thiazole-6-carboxamide;
(S)-N-(chroman-4-yl)-2-(piperazin-1-yl)benzo[d]thiazole-6-carboxamide;
(R)-N-(chroman-4-yl)-2-(piperazin-1-yl)benzo[d]thiazole-6-carboxamide;
(S)-N-(8-fluorochroman-4-yl)-2-(piperazin-1-yl)benzo[d]thiazole-6-carboxamide;
(R)-N-(8-fluorochroman-4-yl)-2-(piperazin-1-yl)benzo[d]thiazole-6-carboxamide;
(S)-N-(7-fluorochroman-4-yl)-2-(piperazin-1-yl)benzo[d]thiazole-6-carboxamide;
(R)-N-(7-fluorochroman-4-yl)-2-(piperazin-1-yl)benzo[d]thiazole-6-carboxamide;
(S)-N-(6-fluorochroman-4-yl)-2-(piperazin-1-yl)benzo[d]thiazole-6-carboxamide;
(S)-N-(5-fluorochroman-4-yl)-2-(piperazin-1-yl)benzo[d]thiazole-6-carboxamide;
(R)-N-(5-fluorochroman-4-yl)-2-(piperazin-1-yl)benzo[d]thiazole-6-carboxamide;
(S)-N-(7-methoxychroman-4-yl)-2-(piperazin-1-yl)benzo[d]thiazole-6-carboxamide;
(R)-N-(7-methoxychroman-4-yl)-2-(piperazin-1-yl)benzo[d]thiazole-6-carboxamide;
(S)-N-(6-methoxychroman-4-yl)-2-(piperazin-1-yl)benzo[d]thiazole-6-carboxamide;
(S)-N-(7-cyanochroman-4-yl)-2-(piperazin-1-yl)benzo[d]thiazole-6-carboxamide;
(S)-N-(2,3-dihydro-1H-inden-1-yl)-2-(piperazin-1-yl)benzo[d]thiazole-6-carboxamide;
(R)-N-(2,3-dihydro-1H-inden-1-yl)-2-(piperazin-1-yl)benzo[d]thiazole-6-carboxamide;
(S)-N-(7-fluoro-2,3-dihydro-1H-inden-1-yl)-2-(piperazin-1-yl)benzo[d]thiazole-6-carboxamide;
(R)-N-(7-fluoro-2,3-dihydro-1H-inden-1-yl)-2-(piperazin-1-yl)benzo[d]thiazole-6-carboxamide;
(S)-N-(6-fluoro-2,3-dihydro-1H-inden-1-yl)-2-(piperazin-1-yl)benzo[d]thiazole-6-carboxamide;
(S)-N-(5-fluoro-2,3-dihydro-1H-inden-1-yl)-2-(piperazin-1-yl)benzo[d]thiazole-6-carboxamide;
(R)-N-(5-fluoro-2,3-dihydro-1H-inden-1-yl)-2-(piperazin-1-yl)benzo[d]thiazole-6-carboxamide;
(S)-N-(4-fluoro-2,3-dihydro-1H-inden-1-yl)-2-(piperazin-1-yl)benzo[d]thiazole-6-carboxamide;
(R)-N-(4-fluoro-2,3-dihydro-1H-inden-1-yl)-2-(piperazin-1-yl)benzo[d]thiazole-6-carboxamide;
(S)-N-(7-methoxy-2,3-dihydro-1H-inden-1-yl)-2-(piperazin-1-yl)benzo[d]thiazole-6-carboxamide;
(S)-N-(6-methoxy-2,3-dihydro-1H-inden-1-yl)-2-(piperazin-1-yl)benzo[d]thiazole-6-carboxamide;
(R)-N-(6-methoxy-2,3-dihydro-1H-inden-1-yl)-2-(piperazin-1-yl)benzo[d]thiazole-6-carboxamide;
(S)-N-(5-methoxy-2,3-dihydro-1H-inden-1-yl)-2-(piperazin-1-yl)benzo[d]thiazole-6-carboxamide;
(S)-N-(4-methoxy-2,3-dihydro-1H-inden-1-yl)-2-(piperazin-1-yl)benzo[d]thiazole-6-carboxamide;
(R)-N-(4-methoxy-2,3-dihydro-1H-inden-1-yl)-2-(piperazin-1-yl)benzo[d]thiazole-6-carboxamide;
(S)-N-(6-cyano-2,3-dihydro-1H-inden-1-yl)-2-(piperazin-1-yl)benzo[d]thiazole-6-carboxamide;
(R)-N-(6-cyano-2,3-dihydro-1H-inden-1-yl)-2-(piperazin-1-yl)benzo[d]thiazole-6-carboxamide;
(S)-N-(5-cyano-2,3-dihydro-1H-inden-1-yl)-2-(piperazin-1-yl)benzo[d]thiazole-6-carboxamide;
(R)-N-(5-cyano-2,3-dihydro-1H-inden-1-yl)-2-(piperazin-1-yl)benzo[d]thiazole-6-carboxamide;
(S)-N-(4-cyano-2,3-dihydro-1H-inden-1-yl)-2-(piperazin-1-yl)benzo[d]thiazole-6-carboxamide;
(R)-N-(4-cyano-2,3-dihydro-1H-inden-1-yl)-2-(piperazin-1-yl)benzo[d]thiazole-6-carboxamide;
N-cyclopentyl-2-(piperazin-1-yl)benzo[d]thiazole-6-carboxamide;
N-cyclohexyl-2-(piperazin-1-yl)benzo[d]thiazole-6-carboxamide;
N-(4,4-difluorocyclohexyl)-2-(piperazin-1-yl)benzo[d]thiazole-6-carboxamide;
(S)-N-(chroman-4-yl)-2-(4-(2-hydroxyethyl)piperazin-1-yl)benzo[d]thiazole-6-carboxamide;
2-(3, 8-diazabicyclo[3.2.1]octan-3-yl)-N-((S)-chroman-4-yl)benzo[d]thiazole-6-carboxamide;
2-(3, 8-diazabicyclo[3.2.1]octan-8-yl)-N-((S)-chroman-4-yl)benzo[d]thiazole-6-carboxamide;
N-(4-chlorobenzyl)-2-(piperidin-4-yl)benzo[d]thiazole-6-carboxamide;
N-(4-fluorobenzyl)-2-(piperidin-4-yl)benzo[d]thiazole-6-carboxamide;
N-(4-methoxybenzyl)-2-(piperidin-4-yl)benzo[d]thiazole-6-carboxamide;
N-(4-cyanobenzyl)-2-(piperidin-4-yl)benzo[d]thiazole-6-carboxamide;
(S)-N-(2,3-dihydro-1H-inden-1-yl)-2-(piperidin-4-yl)benzo[d]thiazole-6-carboxamide;
N-((1R,2R)-2-hydroxy-2,3-dihydro-1H-inden-1-yl)-2-(piperidin-4-yl)benzo[d]thiazole-6-carboxamide;
N-((1S,2S)-2-hydroxy-2,3-dihydro-1H-inden-1-yl)-2-(piperidin-4-yl)benzo[d]thiazole-6-carboxamide;
N-((1R,2S)-2-hydroxy-2,3-dihydro-1H-inden-1-yl)-2-(piperidin-4-yl)benzo[d]thiazole-6-carboxamide;
N-((1S,2R)-2-hydroxy-2,3-dihydro-1H-inden-1-yl)-2-(piperidin-4-yl)benzo[d]thiazole-6-carboxamide;
(S)-N-(chroman-4-yl)-2-(piperidin-4-yl)benzo[d]thiazole-6-carboxamide;
N-isopropyl-2-(piperidin-4-yl)benzo[d]thiazole-6-carboxamide;
N-cyclopentyl-2-(piperidin-4-yl)benzo[d]thiazole-6-carboxamide;
N-cyclohexyl-2-(piperidin-4-yl)benzo[d]thiazole-6-carboxamide;
N-(4,4-difluorocyclohexyl)-2-(piperidin-4-yl)benzo[d]thiazole-6-carboxamide;
N-(3,3-difluorocyclobutyl)-2-(piperidin-4-yl)benzo[d]thiazole-6-carboxamide;
2-(piperidin-4-yl)-N-(tetrahydro-2H-pyran-4-yl)benzo[d]thiazole-6-carboxamide;
(S)-N-(2-methoxy-1-phenylethyl)-2-(piperidin-4-yl)benzo[d]thiazole-6-carboxamide;
(R)-N-(2-methoxy-1-phenylethyl)-2-(piperidin-4-yl)benzo[d]thiazole-6-carboxamide;
(R)-N-(2-hydroxy-1-phenylethyl)-2-(piperidin-4-yl)benzo[d]thiazole-6-carboxamide;
N-benzyl-2-(1-methylpiperidin-4-yl)benzo[d]thiazole-6-carboxamide;
N-(4-chlorobenzyl)-2-(1-methylpiperidin-4-yl)benzo[d]thiazole-6-carboxamide;
N-(4-fluorobenzyl)-2-(1-methylpiperidin-4-yl)benzo[d]thiazole-6-carboxamide;
N-(4-methoxybenzyl)-2-(1-methylpiperidin-4-yl)benzo[d]thiazole-6-carboxamide;
N-(4-cyanobenzyl)-2-(1-methylpiperidin-4-yl)benzo[d]thiazole-6-carboxamide;
N-(4-methylbenzyl)-2-(1-methylpiperidin-4-yl)benzo[d]thiazole-6-carboxamide;
N-((1R,2S)-2-hydroxy-2,3-dihydro-1H-inden-1-yl)-2-(1-methylpiperidin-4-yl)benzo[d]thiazole-6-carboxamide;
N-((1R,2R)-2-hydroxy-2,3-dihydro-1H-inden-1-yl)-2-(1-methylpiperidin-4-yl)benzo[d]thiazole-6-carboxamide;
(S)-N-(chroman-4-yl)-2-(1-methylpiperidin-4-yl)benzo[d]thiazole-6-carboxamide;
N-cyclopentyl-2-(1-methylpiperidin-4-yl)benzo[d]thiazole-6-carboxamide;
2-(1-methylpiperidin-4-yl)-N-(tetrahydro-2H-pyran-4-yl)benzo[d]thiazole-6-carboxamide;
N-cyclohexyl-2-(1-methylpiperidin-4-yl)benzo[d]thiazole-6-carboxamide;
N-(4,4-difluorocyclohexyl)-2-(1-methylpiperidin-4-yl)benzo[d]thiazole-6-carboxamide;
N-isopropyl-2-(1-methylpiperidin-4-yl)benzo[d]thiazole-6-carboxamide;
N-cyclobutyl-2-(1-methylpiperidin-4-yl)benzo[d]thiazole-6-carboxamide;
N-(3,3-difluorocyclobutyl)-2-(1-methylpiperidin-4-yl)benzo[d]thiazole-6-carboxamide;
(S)-N-(2-methoxy-1-phenylethyl)-2-(1-methylpiperidin-4-yl)benzo[d]thiazole-6-carboxamide;
(R)-N-(2-methoxy-1-phenylethyl)-2-(1-methylpiperidin-4-yl)benzo[d]thiazole-6-carboxamide;
(S)-N-(2-hydroxy-1-phenylethyl)-2-(1-methylpiperidin-4-yl)benzo[d]thiazole-6-carboxamide;
(R)-N-(2-hydroxy-1-phenylethyl)-2-(1-methylpiperidin-4-yl)benzo[d]thiazole-6-carboxamide;
N-cyclopentyl-2-(1-ethylpiperidin-4-yl)benzo[d]thiazole-6-carboxamide;
2-(1-ethylpiperidin-4-yl)-N-(tetrahydro-2H-pyran-4-yl)benzo[d]thiazole-6-carboxamide;
2-(1-ethylpiperidin-4-yl)-N-((1R,2R)-2-hydroxy-2,3-dihydro-1H-inden-1-yl)benzo[d]thiazole-6-carboxamide;
2-(1-ethylpiperidin-4-yl)-N-((1S,2S)-2-hydroxy-2,3-dihydro-1H-inden-1-yl)benzo[d]thiazole-6-carboxamide;
2-(1-ethylpiperidin-4-yl)-N-((1R,2S)-2-hydroxy-2,3-dihydro-1H-inden-1-yl)benzo[d]thiazole-6-carboxamide;
2-(1-ethylpiperidin-4-yl)-N-((1S,2R)-2-hydroxy-2,3-dihydro-1H-inden-1-yl)benzo[d]thiazole-6-carboxamide;
(S)-N-(chroman-4-yl)-2-(1-ethylpiperidin-4-yl)benzo[d]thiazole-6-carboxamide;
(S)-N-(2,3-dihydro-1H-inden-1-yl)-2-(1-(2-hydroxyethyl)piperidin-4-yl)benzo[d]thiazole-6-carboxamide;
(S)-N-(chroman-4-yl)-2-(1-(2-hydroxyethyl)piperidin-4-yl)benzo[d]thiazole-6-carboxamide;
N-cyclopentyl-2-(1-(2-hydroxyethyl)piperidin-4-yl)benzo[d]thiazole-6-carboxamide;
2-(1-(2-hydroxyethyl)piperidin-4-yl)-N-(tetrahydro-2H-pyran-4-yl)benzo[d]thiazole-6-carboxamide;
N-cyclohexyl-2-(1-(2-hydroxyethyl)piperidin-4-yl)benzo[d]thiazole-6-carboxamide;
N-(4,4-difluorocyclohexyl)-2-(1-(2-hydroxyethyl)piperidin-4-yl)benzo[d]thiazole-6-carboxamide;
(S)-N-(chroman-4-yl)-2-(1-(2-(2-methoxyethoxy)ethyl)piperidin-4-yl)benzo[d]thiazole-6-carboxamide;
N-((S)-chroman-4-yl)-2-((S)-piperidin-3-yl)benzo[d]thiazole-6-carboxamide;
N-((S)-chroman-4-yl)-2-((R)-piperidin-3-yl)benzo[d]thiazole-6-carboxamide;
N-((S)-chroman-4-yl)-2-(pyrrolidin-3-yl)benzo[d]thiazole-6-carboxamide;
(S)-N-(2,3-dihydro-1H-inden-1-yl)-4-methyl-2-(piperidin-4-yl)benzo[d]thiazole-6-carboxamide;
(S)-N-(2,3-dihydro-1H-inden-1-yl)-2-(1-ethylpiperidin-4-yl)-4-methylbenzo[d]thiazole-6-carboxamide;
N-cyclopentyl-4-methyl-2-(piperidin-4-yl)benzo[d]thiazole-6-carboxamide;
N-cyclopentyl-2-(1-ethylpiperidin-4-yl)-4-methylbenzo[d]thiazole-6-carboxamide;
N-cyclopentyl-4-methyl-2-(1-methylpiperidin-4-yl)benzo[d]thiazole-6-carboxamide;
4-methyl-2-(piperidin-4-yl)-N-(tetrahydro-2H-pyran-4-yl)benzo[d]thiazole-6-carboxamide;
2-(1-ethylpiperidin-4-yl)-4-methyl-N-(tetrahydro-2H-pyran-4-yl)benzo[d]thiazole-6-carboxamide;
(S)-N-(chroman-4-yl)-4-methyl-2-(piperidin-4-yl)benzo[d]thiazole-6-carboxamide;
(S)-N-(chroman-4-yl)-2-(1-ethylpiperidin-4-yl)-4-methylbenzo[d]thiazole-6-carboxamide;
N-cyclopentyl-5-methyl-2-(piperidin-4-yl)benzo[d]thiazole-6-carboxamide;
N-cyclopentyl-2-(1-ethylpiperidin-4-yl)-5-methylbenzo[d]thiazole-6-carboxamide;
(S)-N-(chroman-4-yl)-5-methyl-2-(piperidin-4-yl)benzo[d]thiazole-6-carboxamide;
(S)-N-(chroman-4-yl)-2-(1-ethylpiperidin-4-yl)-5-methylbenzo[d]thiazole-6-carboxamide;
N-cyclopentyl-7-methyl-2-(piperidin-4-yl)benzo[d]thiazole-6-carboxamide;
N-cyclopentyl-2-(1-ethylpiperidin-4-yl)-7-methylbenzo[d]thiazole-6-carboxamide;
(S)-N-(chroman-4-yl)-7-methyl-2-(piperidin-4-yl)benzo[d]thiazole-6-carboxamide;
(S)-N-(chroman-4-yl)-2-(1-ethylpiperidin-4-yl)-7-methylbenzo[d]thiazole-6-carboxamide;
(S)-N-(2,3-dihydro-1H-inden-1-yl)-2-(1-ethylpiperidin-4-yl)-7-methylbenzo[d]thiazole-6-carboxamide;
2-(1-ethylpiperidin-4-yl)-7-methyl-N-(tetrahydro-2H-pyran-4-yl)benzo[d]thiazole-6-carboxamide;
(S)-N-(2,3-dihydro-1H-inden-1-yl)-5-methoxy-2-(piperidin-4-yl)benzo[d]thiazole-6-carboxamide;
N-cyclopentyl-5-methoxy-2-(piperidin-4-yl)benzo[d]thiazole-6-carboxamide;
5-methoxy-2-(piperidin-4-yl)-N-(tetrahydro-2H-pyran-4-yl)benzo[d]thiazole-6-carboxamide;
(S)-N-(chroman-4-yl)-5-methoxy-2-(piperidin-4-yl)benzo[d]thiazole-6-carboxamide;
(S)-N-(2,3-dihydro-1H-inden-1-yl)-4-methyl-2-(piperazin-1-yl)benzo[d]thiazole-6-carboxamide;
(S)-N-(2,3-dihydro-1H-inden-1-yl)-2-(4-ethylpiperazin-1-yl)-4-methylbenzo[d]thiazole-6-carboxamide;
N-cyclobutyl-4-methyl-2-(piperazin-1-yl)benzo[d]thiazole-6-carboxamide;
N-cyclobutyl-4-methyl-2-(4-methylpiperazin-1-yl)benzo[d]thiazole-6-carboxamide;
N-cyclopentyl-4-methyl-2-(piperazin-1-yl)benzo[d]thiazole-6-carboxamide;
N-cyclopentyl-2-(4-ethylpiperazin-1-yl)-4-methylbenzo[d]thiazole-6-carboxamide;
N-cyclopentyl-4-methyl-2-(4-methylpiperazin-1-yl)benzo[d]thiazole-6-carboxamide;
4-methyl-2-(piperazin-1-yl)-N-(tetrahydro-2H-pyran-4-yl)benzo[d]thiazole-6-carboxamide;
2-(4-ethylpiperazin-1-yl)-4-methyl-N-(tetrahydro-2H-pyran-4-yl)benzo[d]thiazole-6-carboxamide;
(S)-N-(chroman-4-yl)-4-methyl-2-(piperazin-1-yl)benzo[d]thiazole-6-carboxamide;
(S)-N-(chroman-4-yl)-2-(4-ethylpiperazin-1-yl)-4-methylbenzo[d]thiazole-6-carboxamide;
N-cyclopentyl-5-methyl-2-(piperazin-1-yl)benzo[d]thiazole-6-carboxamide;
N-cyclopentyl-2-(4-ethylpiperazin-1-yl)-5-methylbenzo[d]thiazole-6-carboxamide;
(S)-N-(chroman-4-yl)-5-methyl-2-(piperazin-1-yl)benzo[d]thiazole-6-carboxamide;
(S)-N-(chroman-4-yl)-2-(4-ethylpiperazin-1-yl)-5-methylbenzo[d]thiazole-6-carboxamide;
N-cyclopentyl-7-methyl-2-(piperazin-1-yl)benzo[d]thiazole-6-carboxamide;
N-cyclopentyl-2-(4-ethylpiperazin-1-yl)-7-methylbenzo[d]thiazole-6-carboxamide;
(S)-N-(chroman-4-yl)-7-methyl-2-(piperazin-1-yl)benzo[d]thiazole-6-carboxamide;
(S)-N-(chroman-4-yl)-2-(4-ethylpiperazin-1-yl)-7-methylbenzo[d]thiazole-6-carboxamide;
N-cyclopentyl-4-methoxy-2-(piperazin-1-yl)benzo[d]thiazole-6-carboxamide;
N-cyclopentyl-2-(4-ethylpiperazin-1-yl)-4-methoxybenzo[d]thiazole-6-carboxamide;
(S)-N-(chroman-4-yl)-4-methoxy-2-(piperazin-1-yl)benzo[d]thiazole-6-carboxamide;
(S)-N-(2,3-dihydro-1H-inden-1-yl)-5-methoxy-2-(piperazin-1-yl)benzo[d]thiazole-6-carboxamide;
N-cyclopentyl-5-methoxy-2-(piperazin-1-yl)benzo[d]thiazole-6-carboxamide;
N-cyclopentyl-2-(4-ethylpiperazin-1-yl)-5-methoxybenzo[d]thiazole-6-carboxamide;
5-methoxy-2-(piperazin-1-yl)-N-(tetrahydro-2H-pyran-4-yl)benzo[d]thiazole-6-carboxamide;
2-(4-ethylpiperazin-1-yl)-5-methoxy-N-(tetrahydro-2H-pyran-4-yl)benzo[d]thiazole-6-carboxamide;
(S)-N-(chroman-4-yl)-5-methoxy-2-(piperazin-1-yl)benzo[d]thiazole-6-carboxamide;
(S)-N-(chroman-4-yl)-4,7-dimethyl-2-(piperidin-4-yl)benzo[d]thiazole-6-carboxamide;
4-chloro-N-cyclopentyl-2-(1-methylpiperidin-4-yl)benzo[d]thiazole-6-carboxamide;
4-cyclopropyl-N-isopropyl-2-(piperidin-4-yl)benzo[d]thiazole-6-carboxamide;
N-cyclobutyl-4-cyclopropyl-2-(1-methylpiperidin-4-yl)benzo[d]thiazole-6-carboxamide;
4-chloro-N-cyclobutyl-2-(piperazin-1-yl)benzo[d]thiazole-6-carboxamide;
4-chloro-N-cyclopentyl-2-(4-(3-hydroxypropyl)piperazin-1-yl)benzo[d]thiazole-6-carboxamide;
2-(3, 8-diazabicyclo[3.2.1]octan-8-yl)-N-cyclopentyl-7-methoxybenzo[d]thiazole-6-carboxamide;
2-(3, 8-diazabicyclo[3.2.1]octan-8-yl)-4-bromo-N-cyclopentylbenzo[d]thiazole-6-carboxamide;
2-(3,8-diazabicyclo[3.2.1]octan-8-yl)-4-cyano-N-isopropylbenzo[d]thiazole-6-carboxamide;
2-(3,8-diazabicyclo[3.2.1]octan-8-yl)-N-cyclopentylbenzo[d]thiazole-6-carboxamide;
2-(3,8-diazabicyclo[3.2.1]octan-8-yl)-N-(4,4-difluorocyclohexyl)benzo[d]thiazole-6-carboxamide;
2-(3-cyclopropyl-3, 8-diazabicyclo[3.2.1]octan-8-yl)-N-(4,4-difluorocyclohexyl)benzo[d]thiazole-6-carboxamide;
N-(4,4-difluorocyclohexyl)-2-(3-(2-hydroxyethyl)-3,8-diazabicyclo[3.2.1]octan-8-yl)benzo[d]thiazole-6-carboxamide;
N-(4,4-difluorocyclohexyl)-2-(3-(3-hydroxypropyl)-3,8-diazabicyclo[3.2.1]octan-8-yl)benzo[d]thiazole-6-carboxamide;
2-(3-oxa-7,9-diazabicyclo[3.3.1]nonan-9-yl)-N-cyclopentylbenzo[d]thiazole-6-carboxamide;
2-(3,8-diazabicyclo[3.2.1]octan-3-yl)-N-(4,4-difluorocyclohexyl)benzo[d]thiazole-6-carboxamide;
N-(4,4-difluorocyclohexyl)-2-(8-methyl-3,8-diazabicyclo[3.2.1]octan-3-yl)benzo[d]thiazole-6-carboxamide;
N-(4,4-difluorocyclohexyl)-2-(8-(2-hydroxyethyl)-3,8-diazabicyclo[3.2.1]octan-3-yl)benzo[d]thiazole-6-carboxamide;
N-(4,4-difluorocyclohexyl)-2-(8-(3-fluoropropyl)-3, 8-diazabicyclo[3.2.1]octan-3-yl)benzo[d]thiazole-6-carboxamide;
2-(3, 8-diazabicyclo[3.2.1]octan-8-yl)-N-cyclopentyl-4-(1-hydroxypropan-2-yl)benzo[d]thiazole-6-carboxamide;
2-(3, 8-diazabicyclo[3.2.1]octan-8-yl)-N-cyclopentyl-4-(2-hydroxyethyl)benzo[d]thiazole-6-carboxamide;
2-(3,6-diazabicyclo[3.1.1]heptan-6-yl)-N-cyclopentylbenzo[d]thiazole-6-carboxamide;
N-cyclopentyl-2-(4,7-diazaspiro[2.5]octan-7-yl)benzo[d]thiazole-6-carboxamide;
N-cyclohexyl-2-((3aR,6aS)-hexahydropyrrolo[3,4-c]pyrrol-2 (1H)-yl)benzo[d]thiazole-6-carboxamide;
N-cyclopentyl-2-(2,6-diazaspiro[3.3]heptan-2-yl)benzo[d]thiazole-6-carboxamide;
N-cyclopentyl-2-(2,6-diazaspiro[3.4]octan-6-yl)benzo[d]thiazole-6-carboxamide;
N-cyclopentyl-2-(2,7-diazaspiro[3.5]nonan-7-yl)benzo[d]thiazole-6-carboxamide;
N-cyclopentyl-2-(octahydro-4H-pyrrolo[3,2-b]pyridin-4-yl)benzo[d]thiazole-6-carboxamide;
N-cyclopentyl-2-(octahydro-5H-pyrrolo[3,2-c]pyridin-5-yl)benzo[d]thiazole-6-carboxamide;
N-cyclopentyl-2-(4-methyl-4,7-diazaspiro[2.5]octan-7-yl)benzo[d]thiazole-6-carboxamide;
N-cyclohexyl-2-((3aR,6aS)-5-methylhexahydropyrrolo[3,4-c]pyrrol-2 (1H)-yl)benzo[d]thiazole-6-carboxamide;
N-cyclopentyl-2-(6-methyl-2,6-diazaspiro[3.3]heptan-2-yl)benzo[d]thiazole-6-carboxamide;
N-cyclopentyl-2-(2-methyl-2,6-diazaspiro[3.4]octan-6-yl)benzo[d]thiazole-6-carboxamide;
N-cyclopentyl-2-(2-methyl-2,7-diazaspiro[3.5]nonan-7-yl)benzo[d]thiazole-6-carboxamide;
N-cyclopentyl-2-(1-methyloctahydro-4H-pyrrolo[3,2-b]pyridin-4-yl)benzo[d]thiazole-6-carboxamide;
N-cyclopentyl-2-(1-methyloctahydro-5H-pyrrolo[3,2-c]pyridin-5-yl)benzo[d]thiazole-6-carboxamide;
(S)-N-(chroman-4-yl)-N-methyl-2-(piperazin-1-yl)benzo[d]thiazole-6-carboxamide;
(S)-N-(chroman-4-yl)-N-methyl-2-(2-methylpyridin-3-yl)benzo[d]thiazole-6-carboxamide;
N-cyclopentyl-N-methyl-2-(piperidin-4-yl)benzo[d]thiazole-6-carboxamide;
2-(piperidin-4-yl)-N-(pyridin-4-ylmethyl)benzo[d]thiazole-6-carboxamide;
2-(piperidin-4-yl)-N-(pyridin-3-ylmethyl)benzo[d]thiazole-6-carboxamide;
N-((1R,2R)-2-hydroxycyclopentyl)-2-(1-methylpiperidin-4-yl)benzo[d]thiazole-6-carboxamide;
2-(1-ethylpiperidin-4-yl)-N-(pyridin-4-ylmethyl)benzo[d]thiazole-6-carboxamide;
2-(1-ethylpiperidin-4-yl)-N-(pyridin-3-ylmethyl)benzo[d]thiazole-6-carboxamide;
2-(1-ethylpiperidin-4-yl)-N-(pyridazin-4-ylmethyl)benzo[d]thiazole-6-carboxamide;
2-(3,8-diazabicyclo[3.2.1]octan-8-yl)-N-(2-isopropoxyethyl)benzo[d]thiazole-6-carboxamide;
2-(3,8-diazabicyclo[3.2.1]octan-8-yl)-N-(2-(cyclopentyloxy)ethyl)benzo[d]thiazole-6-carboxamide;
N-cyclopentyl-2-(1,2-dimethyl-1H-imidazol-5-yl)benzo[d]thiazole-6-carboxamide;
N-cyclopentyl-2-(1-ethylazetidin-3-yl)benzo[d]thiazole-6-carboxamide;
rac-N-cyclopentyl-2-((3S,4R)-3-hydroxy-1-methylpiperidin-4-yl)benzo[d]thiazole-6-carboxamide;
N-cyclopentyl-2-(piperidin-4-yl)benzo[d]thiazole-5-carboxamide;
2-(3,8-diazabicyclo[3.2.1]octan-8-yl)-N-cyclopentylbenzo[d]thiazole-5-carboxamide;
N-cyclopentyl-2-(3-methyl-3, 8-diazabicyclo[3.2.1]octan-8-yl)benzo[d]thiazole-5-carboxamide;
N-cyclopentyl-2-(4-ethylpiperazin-1-yl)benzo[d]thiazole-6-sulfonamide;
N-cyclopentyl-2-(piperidin-4-yl)benzo[d]thiazole-6-sulfonamide;
N-cyclopentyl-2-(1-methylpiperidin-4-yl)benzo[d]thiazole-6-sulfonamide;
2-(3,8-diazabicyclo[3.2.1]octan-8-yl)-N-(4,4-difluorocyclohexyl)benzo[d]thiazole-6-sulfonamide;
N-(4,4-difluorocyclohexyl)-2-(3-ethyl-3,8-diazabicyclo[3.2.1]octan-8-yl)benzo[d]thiazole-6-sulfonamide;
N-(4,4-difluorocyclohexyl)-2-(piperidin-4-yl)benzo[d]thiazole-6-sulfonamide;
N-(4,4-difluorocyclohexyl)-2-(1-(2-hydroxyethyl)piperidin-4-yl)benzo[d]thiazole-6-sulfonamide;
or a pharmaceutically acceptable salt or derivative thereof.

40. A pharmaceutical composition comprising a compound or a pharmaceutically acceptable salt or derivative as defined in claim 15, and a pharmaceutically acceptable excipient.

41. A method for treating or preventing of a disease or disorder that can be ameliorated by activation of long isoforms of PDE4, comprising administering to a patient in need thereof a therapeutically effective amount of a compound or pharmaceutically acceptable salt or derivative of claim 15.

42. (canceled)

43. A method for treating or preventing a disease or disorder mediated by excessive intracellular cyclic AMP signalling comprising administering to a patient in need thereof a therapeutically effective amount of a compound of claim 15.

44. A method of treating or preventing a disease or disorder that can be ameliorated by activation of long isoforms of PDE4, comprising administering to a patient in need thereof a therapeutically effective amount of a compound or a pharmaceutically acceptable salt or derivative of claim 24.

45. A pharmaceutical composition comprising a compound or a pharmaceutically acceptable salt or derivative of claim 39, and a pharmaceutically acceptable excipient.

46. The method of claim 44, wherein disease or disorder that can be ameliorated by activation of long isoforms of PDE4 is a disease or disorder mediated by excessive intracellular cyclic AMP signalling.

47. The method of claim 46, wherein the excessive intracellular cyclic AMP signalling is caused by:
a. excessive hormone levels produced by an adenoma;
b. a gain-of-function gene mutation in a G-protein coupled receptor (GPCR);
c. an activating mutation in the GNAS1 gene, which encodes the α-subunit of the G-protein Gₛ; or
d. a bacterial toxin.

48. The method of claim 1, wherein the disease is cancer, optionally wherein the cancer is prostate cancer.

49. The method of claim 1, wherein the disease is:
a. pituitary adenoma, Cushing's disease, polycystic kidney disease or polycystic liver disease;
b. hyperthyroidism, Jansens's metaphyseal chondrodysplasia, hyperparathyroidism, or familial male-limited precocious puberty;
c. McCune-Albright syndrome;
d. cholera, whooping cough, anthrax, or tuberculosis;
e. HIV, AIDS, or Common Variable Immunodeficiency (CVID);
f. melanoma, pancreatic cancer, leukaemia, prostate cancer, adrenocortical tumours, testicular cancer, primary pigmented nodular adrenocortical diseases (PPNAD), or Carney Complex;
g. autosomal dominant polycystic kidney disease (ADPKD) or autosomal recessive polycystic kidney disease (ARPKD); or
h. maturity onset diabetes of young type 5 (MODY5); or
i. cardiac hypertrophy.

50. The method of claim 49, wherein the disease is:
a. autosomal dominant polycystic kidney disease (ADPKD), autosomal recessive polycystic kidney disease (ARPKD); or
b. hyperparathyroidism.
